(19) United States
(12) Patent Application Publication
(10) Pub. No.: US 2025/0059207 A1
(43) Pub. Date: Feb. 20, 2025

(54) 6,6-FUSED HETEROCYCLIC COMPOUNDS AS METTL3 INHIBITORS

(71) Applicant: 858 Therapeutics, Inc., San Diego, CA (US)

(72) Inventors: Zacharia S. CHERUVALLATH, San Diego, CA (US); James M. VEAL, Solana Beach, CA (US); Jeffrey A. STAFFORD, Rancho Santa Fe, CA (US); Christopher MCBRIDE, San Diego, CA (US)

(21) Appl. No.: 18/718,735

(22) PCT Filed: Dec. 27, 2022
(86) PCT No.: PCT/US2022/082436

§ 371 Date: Jun. 11, 2024

Related U.S. Application Data
(60) Provisional application No. 63/294,674, filed on Dec. 29, 2021.

(51) Int. Cl. C07D 519/00; A61K 31/4375; A61K 31/438; A61K 31/444; A61K 31/4545; A61K 31/4709; A61K 31/496; A61K 31/4995; A61K 31/502; A61K 31/5025; A61K 31/519; A61K 31/5377; A61K 31/538; A61K 31/5386; A61P 35/02; C07D 471/04

(52) U.S. Cl. CPC C07D 519/00; A61K 31/4375; A61K 31/438; A61K 31/444; A61K 31/4545; A61K 31/4709; A61K 31/496; A61K 31/4995; A61K 31/502; A61K 31/5025; A61K 31/519; A61K 31/5377; A61K 31/538; A61K 31/5386; A61P 35/02; C07D 471/04

(57) ABSTRACT

The present disclosure relates to 6,6-fused heterocyclic compounds and related compounds and their use in treating a disease or condition responsive to modulation or inhibition of METTL3.

### CROSS-REFERENCE TO RELATED APPLICATION

This application claims the benefit of U.S. Patent Application No. 63/294,674, filed on Dec. 29, 2021, which is hereby incorporated by reference in its entirety.

### FIELD OF DISCLOSURE

The present disclosure relates to compounds comprising a 6,6-fused heterocyclic core, their use for modulating or inhibiting methyltransferase-like 3 (METTL3) pathways such as incorporating N6-methyladenosine (m6A) in transcripts in the transcriptome, and their use in therapeutic methods of treating conditions and disorders.

### BACKGROUND

Chemical modifications of RNA have essential role in a vast range of cellular processes. N6-methyladenosine (m6A) is an abundant internal modification in messenger RNA (mRNA) and long non-coding RNA that can be dynamically added and removed by RNA methyltransferases(MTases) and demethylases, respectively.

Methyltransferase-like 3 (METTL3) is the enzyme responsible for forming nearly all m6A in mRNA and other transcripts generated by RNA polymerase II, such as many long noncoding RNAs. The function of METTL3 is to post-transcriptionally methylate adenosines in specific transcripts within the transcriptome. Therefore, modulators or inhibitors of METTL3 could be useful to regulate nuclear export, stability, splicing and translation of specific transcripts. These effects are most likely to be prominent on transcripts that contain m6A in the cell type of interest. Since the effects of m6A may differ between different transcripts, the effects of METTL3 modulation or inhibition may be different for different transcripts.

Studies of METTL3 have revealed involvement in various pathologies. METTL3 is involved in proliferation and growth of various cancers and in regulating immune responses to tumors (since at least one m6A-binding protein can suppress anti-tumor responses). METTL3 is also involved in diseases in which stress granules have a pathogenic role, including a variety of different neurological diseases such as ALS. Further, certain studies have shown that METTL3 can promote viral propagation.

M6A is highly abundant in interferon beta mRNA and potentially other transcripts that encode for proteins involved in inflammation. As a result, these transcripts are unstable and these inflammatory signals are normally low in cells. METTL3 modulators or inhibitors can therefore increase the expression of these transcripts and therefore enhance inflammatory responses, which may be beneficial in anti-tumor functions, and also in antiviral functions or other circumstances where enhancing inflammation is beneficial.

One of the functions of m6A is to regulate cellular differentiation—m6A can regulate the expression of various mRNAs linked to cell fate. Modulators or inhibitors of METTL3 are involved in regulating the expression of these mRNAs, which can affect cell fate and thus alter diseases where there is altered or pathologic differentiation.

Therefore, modulating or inhibiting METTL3 is a potential therapeutic approach for treating disorders such as neurological diseases, viral diseases, cancer, and pre-cancerous syndromes. Thus, there is a need for compounds that can modulate or inhibit METTL3.

### SUMMARY

The present disclosure provides a compound of Formula (I′):

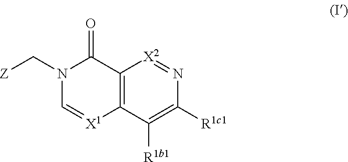

or a pharmaceutically acceptable salt thereof, wherein:
X¹ is CR^{1a} or N;
R^{1a} is selected from the group consisting of H, C₁₋₆alkyl, halo, cycloalkyl, aryl, and heteroaryl; wherein the C₁₋₆alkyl of R^{1a} is unsubstituted or substituted with aryl or heteroaryl, wherein the aryl and heteroaryl is unsubstituted or substituted with one or more substituents selected from the group consisting of of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NR^{1e}C(O)R^{1e};
X² is CH or N;
R^{1b1} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b1} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, —CH(N(R^{1e})₂)COOR^{1e} and —NR^{1e}C(O)R^{1e};
wherein the C₁₋₆alkyl and alkynyl of R^{1b1} is unsubstituted or substituted with one or more substituents selected from the group consisting of halo, aryl, heteroaryl, heterocyclyl, and cycloalkyl;
R^{1c1} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;

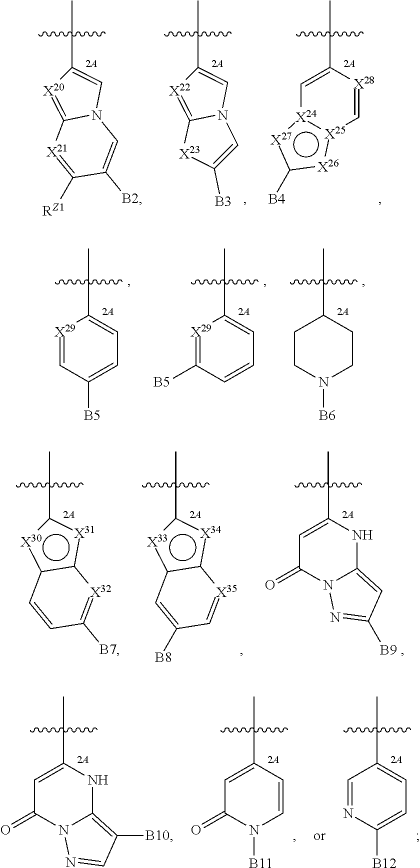

X²⁰ is CR^{20a} or N; wherein R^{20a} is H, halo, CN, aryl, or heteroaryl;
X²¹ is CH or N;
X²² is CR^{22a} or N; wherein R^{22a} is H, halo, CN, aryl, or heteroaryl;
X²³ is CH₂, S, O, NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
X²⁹ is CH or N;
X³⁰ and X³¹ are independently CH, O, S, N, or NH;
X³² is CH or N;
X³³ and X³⁴ are independently CH, O, S, N, or NH;
X³⁵ is CH or N;
R^{Z1} is H, halo, —CN, or —S(O)₂F;
B2, B3, B4, B7, B8, B9, and B10 are independently selected from the group consisting of H, C₁₋₆alkyl, CN, —S(O)₂F, —CH₂—NR^{2a}R^{2b},

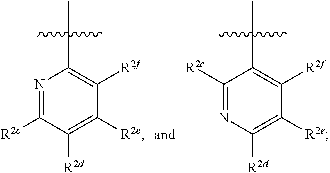

R^{2a} is H or C₁₋₆alkyl;
R^{2b} is selected from the group consisting of H, C₁₋₆alkyl, aryl, heteroaryl, heterocyclyl, —C(NH)-aryl, —S(O)(NH)R^{2g}, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, —S(O)₂F, —S(O)(NH)R^{2g}, alkoxy, aryl, haloaryl, cycloalkyl, halocycloalkyl, hydroxycycloalkyl, and heterocyclyl, wherein the cycloalkyl is unsubstituted or substituted with one or more substituents selected from the group consisting of C₁₋₆alkyl, —(CH₂)ₙF, —(CH₂)ₙBr, or —(CH₂)ₙCN and wherein the heterocyclyl is unsubstituted or substituted with C₁₋₆alkyl; or
R^{2a} and R^{2b} combine to form a 5-7-membered heterocyclyl, wherein the heterocyclyl is unsubstituted or substituted with one or more substituents selected from the group consisting of C₁₋₆alkyl and cycloalkyl;
R^{2c}, R^{2d}, R^{2e}, and R^{2f} are each independently H, C₁₋₆alkyl, heterocycyl, or —NHR^{2g};
R^{2g} is selected from the group consisting of H, C₁₋₆alkyl, and cycloalkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;

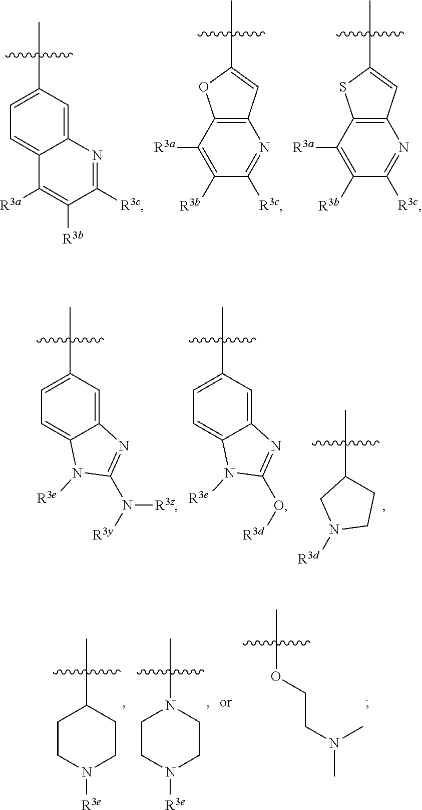

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, —OR^{3x′}, and —CO₂R^{x};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

The present disclosure provides a compound of Formula (I):

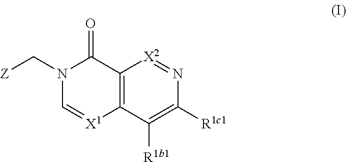

or a pharmaceutically acceptable salt thereof, wherein:
X¹ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X² is CH or N;
R^{1b1} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b1} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b1} is unsubstituted or substituted with one or more substituents selected from the group consisting of aryl, heterocyclyl, and cycloalkyl; R^{1c1} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl; wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
Z is

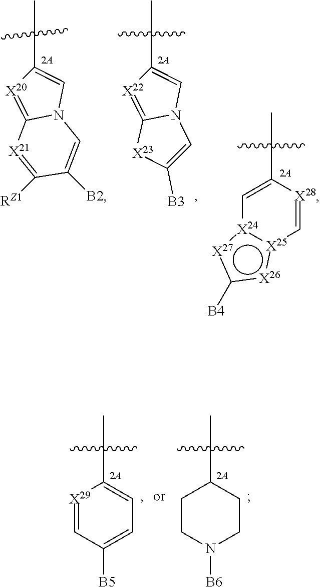

X²⁰ is CR^{20a} or N; wherein R^{20a} is H, halo, CN, aryl, or heteroaryl;

X²¹ is CH or N;
X²² is CR^{22a} or N; wherein R^{22a} is H, halo, CN, aryl, or heteroaryl;
X²³ is CH₂, S, O, NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
X²⁹ is CH or N;
B2, B3, and B4 are independently selected from the group consisting of H, C₁₋₆alkyl, CN, —CH₂—NR^{2a}R^{2b},

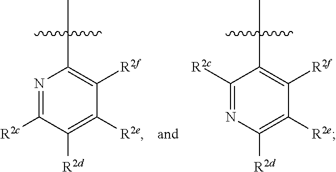

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;

B5 and B6 are independently

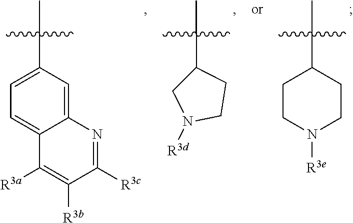

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

The present disclosure also provides a compound of Formula (II):

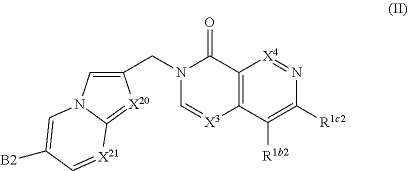

or a pharmaceutically acceptable salt thereof, wherein:
X³ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X⁴ is CH or N;
R^{1b2} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b2} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b2} is unsubstituted or substituted with one or more substituents selected from the group consisting of aryl, heterocyclyl, and cycloalkyl;
R^{1c2} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl; wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁰ is CR^{20a} or N; wherein R^{20a} is H, halo, CN, aryl, or heteroaryl;
X²¹ is CH or N;
B2 is selected from the group consisting of H, C₁₋₆alkyl, CN, —CH₂—NR^{2a}R^{2b},

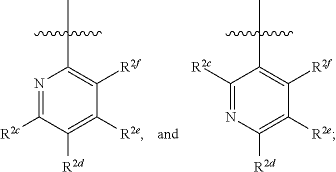

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

In some embodiments, the compound of Formula (II), or the pharmaceutically acceptable salt thereof, is a compound of Formula (IIa), or a pharmaceutically acceptable salt thereof:

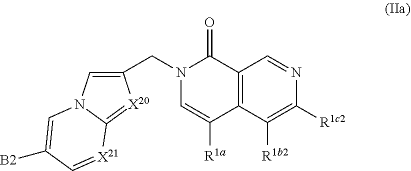

wherein R^{1a}, R^{1b2}, R^{1c2}, X²⁰, X²¹, and B2 are as described for Formula (II).

In some embodiments, the compound of Formula (II), or the pharmaceutically acceptable salt thereof, is a compound of Formula (IIb), or a pharmaceutically acceptable salt thereof:

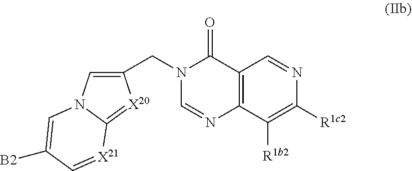

wherein R^{1b2}, R^{1c2}, X²⁰, X²¹, and B2 are as described for Formula (II).

In some embodiments, the compound of Formula (II), or the pharmaceutically acceptable salt thereof, is a compound of Formula (IIc), or a pharmaceutically acceptable salt thereof:

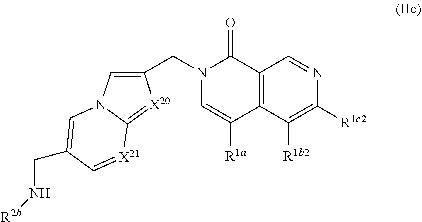

wherein R^{1a}, R^{1b2}, R^{1c2}, and R^{2b} are as described for Formula (II).

The present disclosure also a compound of Formula (III):

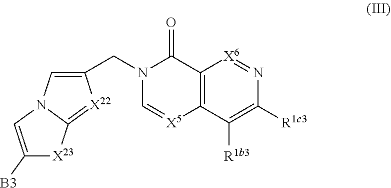

or a pharmaceutically acceptable salt thereof, wherein:
X⁵ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X⁶ is CH or N;
R^{1b3} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b3} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b3} is unsubstituted or substituted with one or more substituents selected from the group consisting of aryl, heterocyclyl, and cycloalkyl;
R^{1c3} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²² is CR^{22a} or N; wherein R^{22a} is H, halo, aryl, or heteroaryl;
X²³ is CH, S, O, or NH;
B3 is selected from the group consisting of H, C₁₋₆alkyl, CN, —CH₂—NR^{2a}R^{2b},

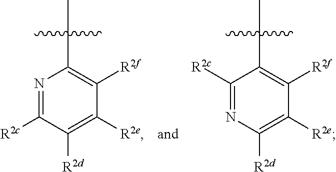

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

In some embodiments, the compound of Formula (III), or the pharmaceutically acceptable salt thereof, is a compound of Formula (IIIa), or a pharmaceutically acceptable salt thereof:

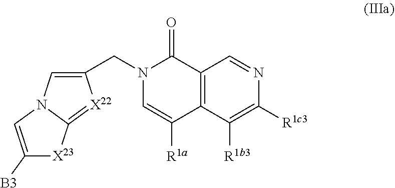

wherein R^{1a}, R^{1b3}, R^{1c3}, X²², X²³, and B3 are as described for Formula (III).

In some embodiments, the compound of Formula (III), or the pharmaceutically acceptable salt thereof, is a compound of Formula (IIIb), or a pharmaceutically acceptable salt thereof:

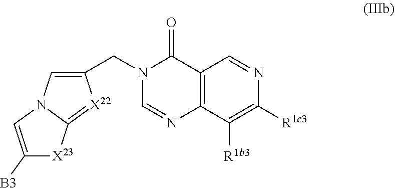

wherein R^{1b3}, R^{1c3}, X²², X²³, and B3 are as described for Formula (III).

In some embodiments, the compound of Formula (III), or the pharmaceutically acceptable salt thereof, is a compound of Formula (IIIc), or a pharmaceutically acceptable salt thereof:

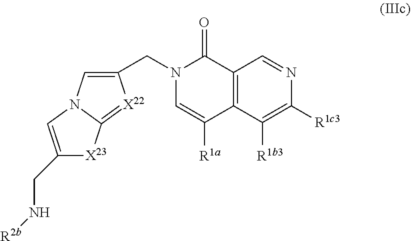

wherein R^{1a}, R^{1b3}, R^{1c3}, R^{2b}, X²², and X²³ are as described for Formula (III).

The present disclosure also provides a compound of Formula (IV):

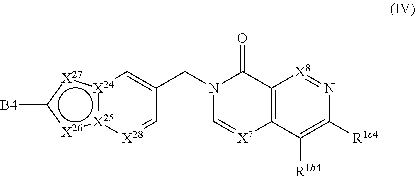

or a pharmaceutically acceptable salt thereof, wherein:
X⁷ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X⁸ is CH or N;
R^{1b4} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b4} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1c}, —(CH₂)ₘNH(CH₂)ₘS(O)₂R^{1e}, and —NHC(O)R^{1e};
wherein the C₁₋₆alkyl and alkynyl of R^{1b4} is unsubstituted or substituted with one or more substituents selected from the group consisting of aryl, heterocyclyl, and cycloalkyl;
R^{1c4} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl; wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
B4 is selected from the group consisting of H, C₁₋₆alkyl, CN, —CH₂—NR^{2a}R^{2b},

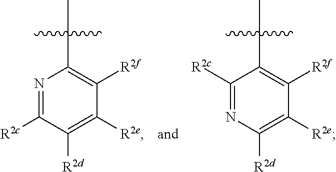

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

In some embodiments, the compound of Formula (IV), or the pharmaceutically acceptable salt thereof, is a compound of Formula (IVa), or a pharmaceutically acceptable salt thereof:

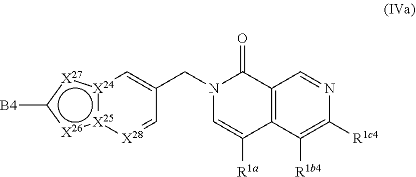

wherein R^{1a}, R^{1b4}, R^{1c4}, X²⁴, X²⁵, X²⁶, X²⁷, X²⁸, and B4 are as described for Formula(IV).

In some embodiments, the compound of Formula (IV), or the pharmaceutically acceptable salt thereof, is a compound of Formula (IVb), or a pharmaceutically acceptable salt thereof:

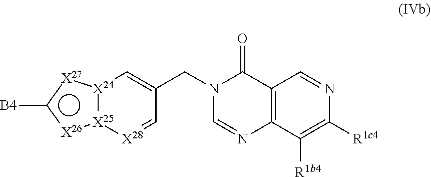

wherein R^{1b4}, R^{1c4}, X²⁴, X²⁵, X²⁶, X²⁷, X²⁸, and B4 are as described for Formula(IV).

In some embodiments, the compound of Formula (IV), or the pharmaceutically acceptable salt thereof, is a compound of Formula (IVc), or a pharmaceutically acceptable salt thereof:

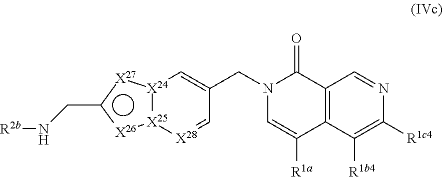

wherein R^{1a}, R^{1b4}, R^{1c4}, R^{2b}, X²⁴, X²⁵, X²⁶, X²⁷, and X²⁸ are as described for Formula (IV).

The present disclosure also provides a compound of Formula (V):

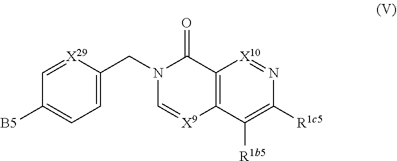

or a pharmaceutically acceptable salt thereof, wherein:
X⁹ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X¹⁰ is CH or N;
R^{1b5} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b5} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b5} is unsubstituted or substituted with one or more substituents selected from the group consisting of aryl, heterocyclyl, and cycloalkyl;
R^{1c5} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁹ is CH or N; B⁵ is

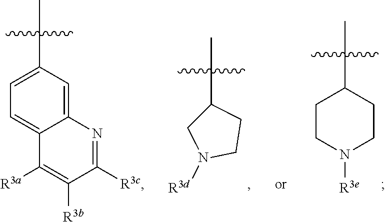

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

In some embodiments, the compound of Formula (V), or the pharmaceutically acceptable salt thereof, is a compound of Formula (Va), or a pharmaceutically acceptable salt thereof:

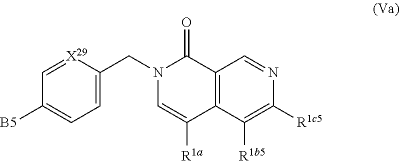

wherein R^{1a}, R^{1b5}, R^{1c5}, X²⁹, and B5 are as described for Formula (V).

In some embodiments, the compound of Formula (V), or the pharmaceutically acceptable salt thereof, is a compound of Formula (Vb), or a pharmaceutically acceptable salt thereof:

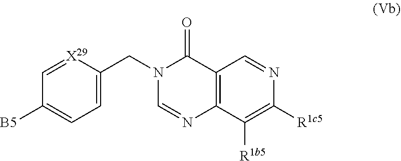

wherein R^{1b5}, R^{1c5}, X²⁹, and B5 are as described for Formula(V).

The present disclosure also provides a compound of Formula (VI):

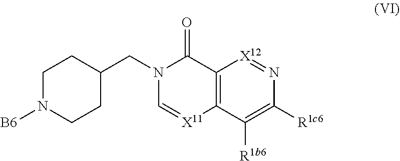

or a pharmaceutically acceptable salt thereof, wherein:
X¹¹ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X¹² is CH or N;
R^{1b6} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b6} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b6} is unsubstituted or substituted with one or more substituents selected from the group consisting of aryl, heterocyclyl, and cycloalkyl;
R^{1c6} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
B6 is

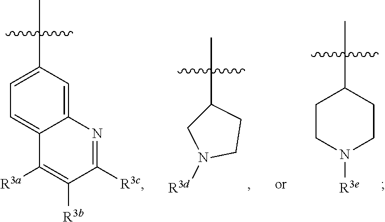

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

In some embodiments, the compound of Formula (VI), or the pharmaceutically acceptable salt thereof, is a compound of Formula (VIa), or a pharmaceutically acceptable salt thereof:

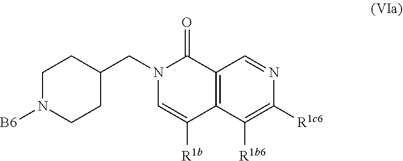

wherein R^{1b}, R^{1b6}, R^{1c6}, and B6 are as described for Formula (VI).

In some embodiments, the compound of Formula (VI), or the pharmaceutically acceptable salt thereof, is a compound of Formula (VIb), or a pharmaceutically acceptable salt thereof:

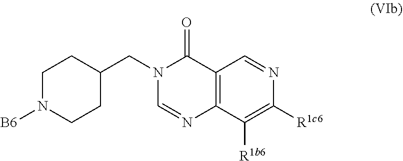

wherein R^{1b6}, R^{1c6}, and B6 are as described for Formula (VI).

Another embodiment provides a compound of Formula (VII), or a pharmaceutically acceptable salt thereof, having the structure:

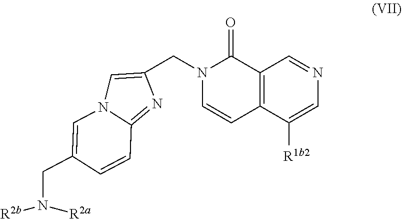

wherein:
R^{1b2} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b2} is unsubstituted or substituted with one or more substituents selected from the group consisting of C₁₋₆alkyl, halo, —CN, —(CH₂)ₘN(R^{1e})₂, —(CH₂)ₘOH, —C(O)R^{1e}, —C(O)N(R^{1e})₂, —(CH₂)ₘCOOH, aryl, heteroaryl, heterocyclyl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b2} is unsubstituted or substituted with one or more substituents selected from the group consisting of aryl, heteroaryl, heterocyclyl, and cycloalkyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
R^{2a} is H or C₁₋₆alkyl;
wherein the C₁₋₆alkyl of R^{2a} is unsubstituted or substituted with one or more substituents selected from the group consisting of halogen, —OH, —CN, —N(R^{2h})₂, alkoxy, cycloalkyl, halocycloalkyl, hydroxycycloalkyl, and heterocyclyl;
R^{2b} is selected from the group consisting of C₁₋₆alkyl, heterocyclyl, cycloalkyl, heterocyclylalkyl, and cycloalkylalkyl;
wherein the C₁₋₆alkyl, heterocyclyl, cycloalkyl, heterocyclylalkyl, and cycloalkylalkyl of R^{2b} is unsubstituted or substituted with one or more substituents selected from the group consisting of C₁₋₆alkyl, C₁₋₆haloalkyl, halogen, —OH, —CN, —N(R^{2h})₂, alkoxy, aryl, haloaryl, cycloalkyl, halocycloalkyl, hydroxycycloalkyl, and heterocyclyl; or R^{2a} and R^{2b} combine to form a 5-8-membered heterocyclyl;
each R^{2h} is independently H, C₁₋₆alkyl, or —(CH₂)ₙOCH₃; and
n is 1, 2, 3, 4, 5, or 6.

The present disclosure provides a pharmaceutical composition comprising a compound of the present disclosure, and pharmaceutically acceptable salts thereof, and a pharmaceutically acceptable carrier.

The present disclosure provides a compound of the present disclosure, and pharmaceutically acceptable salts thereof, for use in the treatment of a disorder that is responsive to modulation or inhibition of METTL3.

The present disclosure provides use of a compound of the present disclosure, and pharmaceutically acceptable salts thereof, in the treatment of a disorder that is responsive to modulation or inhibition of METTL3.

The present disclosure provides a compound of the present disclosure, and pharmaceutically acceptable salts thereof, for use in the manufacture of a medicament for the treatment of a disorder that is responsive to modulation or inhibition of METTL3.

The present disclosure provides a method of treating a disorder in a subject in need thereof, wherein the disorder is mediated by METTL3, comprising administering to the subject an effective amount of a compound of the present disclosure, and pharmaceutically acceptable salts thereof.

### DETAILED DESCRIPTION

The following description sets forth numerous exemplary configurations, methods, parameters, and the like. It should be recognized, however, that such description is not intended as a limitation on the scope of the present disclosure, but is instead provided as a description of exemplary embodiments.

As used herein, the terms “including,” “containing,” and “comprising” are used in their open, non-limiting sense.

The articles “a” and “an”, as used herein, refer to one or more than one (i.e., to at least one) of the grammatical object of the article. By way of example, “an element” refers to one element or more than one element.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with resp ect to the maximum amount of the same entity that could be obtained under the particular stoichiometric conditions. Concentrations that are given as percentages refer to mass ratios, unless indicated differently.

“Optional” or “optionally”, as used herein, means that the subsequently described event or circumstance may or may not occur, and that the description includes instances where the event or circumstance occurs and instances in which it does not.

“Alkyl”, as used herein, refers to an unbranched or branched saturated hydrocarbon chain. Alkyl can be used alone, or as part of another radical, such as cycloalkyl-alkyl. In some embodiments, alkyl as used herein has 1 to 50 carbon atoms ((C₁₋₅₀)alkyl), 1 to 20 carbon atoms ((C₁₋₂₀)alkyl), 1 to 12 carbon atoms ((C₁₋₁₂)alkyl), 1 to 10 carbon atoms ((C₁₋₁₀)alkyl), 1 to 8 carbon atoms ((C₁₋₈)alkyl), 1 to 6 carbon atoms ((C₁₋₆)alkyl), 1 to 4 carbon atoms((C₁₋₄)alkyl), or 1 to 3 carbon atoms ((C₁₋₃)alkyl). In other embodiments, alkyl as used herein has 2 to 6 carbon atoms ((C₂₋₆)alkyl). Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methyl pentyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

“Alkenyl”, as used herein, refers to an unbranched or branched hydrocarbon chain containing 2-12 carbon atoms. The “alkenyl” group contains at least one double bond. The double bond of an alkenyl group can be unconjugated or conjugated to another unsaturated group. Examples of alkenyl groups include, but are not limited to, ethylenyl, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl and the like. An alkenyl group can be unsubstituted or substituted. The alkenyl may be branched or straight.

“Alkynyl”, as used herein, refers to an unbranched or branched unsaturated hydrocarbon chain containing 2-12 carbon atoms. The “alkynyl” group contains at least one triple bond. The triple bond of an alkynyl group can be unconjugated or conjugated to another unsaturated group. Examples of alkynyl groups include, but are not limited to, ethynyl, propynyl, butynyl, pentynyl, hexynyl, methylpropynyl, 4-methyl-1-butynyl, 4-propyl-2-pentynyl, 4-butyl-2-hexynyl and the like. An alkynyl group can be unsubstituted or substituted.

“Alkoxy”, as used herein, refers to a straight or branched chain saturated hydrocarbon containing 1-12 carbon atoms containing a terminal “O” in the chain, i.e., —O(alkyl). Examples of alkoxy groups include, without limitation, methoxy, ethoxy, propoxy, butoxy, t-butoxy, or pentoxy groups.

“Cycloalkyl”, as used herein, refers to a saturated or partially saturated, monocyclic, fused or spiro polycyclic, carbocycle having from 3 to 18 carbon atoms per ring. The cycloalkyl ring or carbocycle may be optionally substituted by one or more substituents, e.g., 1 to 5 substituents, at any point of attachment. The substituents can themselves be optionally substituted. Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, bicyclo[2.2.2]octenyl, decahydronaphthalenyl, octahydro-1H-indenyl, cyclopentenyl, cyclohexenyl, cyclohexa-1,4-dienyl, cyclohexa-1,3-dienyl, 1,2,3,4-tetrahydronaphthalenyl, octahydropentalenyl, 3a, 4,5,6,7,7a-hexahydro-1H-indenyl, 1,2,3,3a-tetrahydropentalenyl, bicyclo[3.1.0]hexanyl, bicyclo[2.1.0]pentanyl, spiro[3.3]heptanyl, bicyclo[2.2.1]heptanyl, bicyclo[2.2.1]hept-2-enyl, bicyclo[2.2.2]octanyl, 6-methylbicyclo[3.1.1]heptanyl, 2,6,6-trimethylbicyclo[3.1.1]heptanyl, and derivatives thereof.

“Cycloalkenyl”, as used herein, refers to a partially saturated, monocyclic or fused or spiro polycyclic carbocycle having from 3 to 18 carbon atoms per ring and containing at least one double bond. The cycloalkenyl ring may be optionally substituted by one or more substituents, e.g., 1 to 5 substituents, at any point of attachment. The substituents can themselves be optionally substituted.

“Heterocycloalkyl” or “heterocyclyl”, as used herein, refers to a saturated or partially unsaturated and non-aromatic monocyclic or fused or spiro polycyclic ring structure of 4- to- 18 atoms containing carbon and heteroatoms taken from oxygen, nitrogen, or sulfur wherein there is not delocalized π-electrons (aromaticity) shared among the ring carbon or heteroatoms. The heterocycloalkyl or heterocyclyl ring structure may be substituted by one or more substituents. The substituents can themselves be optionally substituted. Examples of heterocycloalkyl or heterocyclyl rings include, but are not limited to, oxetanyl, azetidinyl, tetrahydrofuranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, homotropanyl, dihydrothiophen-2(3H)-onyl, tetrahydrothiophene 1,1-dioxide, 2,5-dihydro-1H-pyrrolyl, imidazolidin-2-one, pyrrolidin-2-one, dihydrofuran-2(3H)-one, 1,3-dioxolan-2-one, isothiazolidine 1,1-dioxide, 4,5-dihydro-1H-imidazolyl, 4,5-dihydrooxazolyl, oxiranyl, pyrazolidinyl, 4H-1,4-thiazinyl, thiomorpholinyl, 1,2,3,4-tetrahydropyridinyl, 1,2,3,4-tetrahydropyrazinyl, 1,3-oxazinan-2-one, tetrahydro-2H-thiopyran 1,1-dioxide, 7-oxabicyclo[2.2.1]heptanyl, 1,2-thiazepane 1,1-dioxide, octahydro-2H-quinolizinyl, 1,3-diazabicyclo[2.2.2]octanyl, 2,3-dihydrobenzo[b][1,4]dioxine, 3-azabicyclo[3.2.1]octanyl, 8-azaspiro[4.5]decane, 8-oxa-3-azabicyclo[3.2.1]octanyl, 2-azabicyclo[2.2.1]heptane, 2,8-diazaspiro[5.5]undecanyl, 2-azaspiro[5.5]undecanyl, 3-azaspiro[5.5]undecanyl, decahydroisoquinolinyl, 1-oxa-8-azaspiro[4.5]decanyl, 8-azabicyclo[3.2.1]octanyl, 1,4′-bipiperidinyl, azepanyl, 8-oxa-3-azabicyclo[3.2.1]octanyl, 3,4-dihydro-2H-benzo[b][1,4]oxazinyl, 5,6,7,8-tetrahydroimidazo[1,2-a]pyridinyl, 1,4-diazepanyl, phenoxathiinyl, benzo[d][1,3]dioxolyl, 2,3-dihydrobenzofuranyl, 2,3-dihydrobenzo[b][1,4]dioxinyl, 4-(piperidin-4-yl)morpholinyl, 3-azaspiro[5.5]undecanyl, decahydroquinolinyl, piperazin-2-one, 1-(pyrrolidin-2-ylmethyl)pyrrolidinyl, 1,3′-bipyrrolidinyl, and 6,7,8,9-tetrahydro-1H, 5H-pyrazolo[1,2-a][1,2]diazepinyl.

“Aryl”, as used herein, refers to a monocyclic or polycyclic group having at least one hydrocarbon aromatic ring wherein all of the ring atoms of the at least one hydrocarbon aromatic ring are carbon. Aryl may include groups with a single aromatic ring (e.g., phenyl) and multiple fused aromatic rings (e.g., naphthyl, anthryl). Aryl may further include groups with one or more aromatic hydrocarbon rings fused to one or more non-aromatic hydrocarbon rings (e.g., fluorenyl; 2,3-dihydro-1H-indene; 1,2,3,4-tetrahydronaphthalene). In certain embodiments, aryl includes groups with an aromatic hydrocarbon ring fused to a non-aromatic ring wherein the non-aromatic ring comprises at least one ring hetero atom independently selected from the group consisting of N, O, and S. For example, in some embodiments, aryl includes groups with a phenyl ring fused to a non-aromatic ring, wherein the non-aromatic ring comprises at least one ring hetero atom independently selected from the group consisting of N, O, and S(e.g., chromane; thiochromane; 2,3-dihydrobenzofuran; indoline). In some embodiments, aryl as used herein has from 6 to 14 carbon atoms ((C₆-C₁₄)aryl), or 6 to 10 carbon atoms ((C₆-C₁₀)aryl). Where the aryl includes fused rings, the aryl may connect to one or more substituents or moieties of the formulae described herein through any atom of the fused ring for which valency permits. The aryl ring structure may be substituted by one or more substituents. The substituents can themselves be optionally substituted.

“Heteroaryl”, as used herein, refers to a monocyclic or polycyclic group comprising at least one aromatic ring, wherein the aromatic ring comprises at least one ring heteroatom independently selected from the group consisting of N, O, and S. The heteroaryl group may comprise 5, 6, 7, 8, 9, 10, 11, 12, or more ring atoms, where ring atoms refer to the sum of carbon and heteroatoms in the one or more rings (e.g., be a 5-membered, 6-membered, 7-membered, 8-membered, 9-membered, 10-membered, 11-membered, or 12-membered heteroaryl). In some embodiments, heteroaryl includes groups with an aromatic ring that comprises at least one ring heteroatom independently selected from the group consisting of N, O, and S, (e.g., pyridinyl, pyrazinyl, furanyl, thiophenyl). In certain embodiments, heteroaryl includes polycyclic groups with an aromatic ring comprising at least one ring heteroatom, fused to a non-aromatic hydrocarbon ring (e.g., 5,6,7,8-tetrahydroquinolinyl; 4,5,6,7-tetrahydroisobenzofuranyl). In some embodiments, heteroaryl includes polycyclic groups with an aromatic ring comprising at least one ring heteroatom fused to an aromatic hydrocarbon ring (e.g., quinolinyl, quinoxalinyl, benzothiazolyl). In still further embodiments, heteroaryl includes polycyclic groups with two fused aromatic rings, wherein each ring comprises at least one ring heteroatom (e.g., naphthyridinyl). Heteroaryl may include groups comprising 1 to 5 ring heteroatoms, 1 to 4 ring heteroatoms, 1 to 3 ring heteroatoms, 1 or 2 ring heteroatoms, or 1 ring heteroatom, wherein each ring heteroatom is independently selected from the group consisting of N, O, and S. In one example, a heteroaryl has 3 to 8 ring carbon atoms, with 1 to 3 ring heteroatoms independently selected from N, O, and S. Examples of heteroaryl groups include, without limitations, pyridyl, pyridazinyl, pyrimidinyl, benzothiazolyl, and pyrazolyl. The heteroaryl ring structure may be substituted by one or more substituents. The substituents can themselves be optionally substituted.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

“Amino”, as used herein, means a substituent containing at least one nitrogen atom. For example, NH₂, —NH(alkyl) or alkylamino, —N(alkyl)₂ or dialkylamino, amide, carboxamide, urea, and sulfamide are included in the term “amino”.

“Cyano”, as used herein, refers to a substituent having a carbon atom joined to a nitrogen atom by a triple bond, i.e., c [symbol] N.

“Hydroxyl” or “hydroxy”, as used herein, refers to an OH group.

“Hydroxycycloalkyl”, as used herein, refers to an cycloalkyl group substituted with one or more hydroxy.

“Halo”, as used herein, refers to fluoro, chloro, bromo, or iodo radicals.

“Halocycloalkyl”, as used herein, refers to an cycloalkyl group substituted with one or more halogen.

“Haloaryl”, as used herein, refers to an aryl group substituted with one or more halogen.

“Haloalkoxy”, as used herein, refers to an alkoxy group which is substituted with one or more halogen. Examples of haloalkyl groups include, but are not limited to, trifluoromethoxy, difluoromethoxy, pentafluoroethoxy, trichloromethoxy, etc.

“Oxo”, as used herein, refers to an “═O” group.

It should be understood that when a range of values is listed, it is intended to encompass each value and sub-range within the range. For example, “C₁₋₆alkyl” (which may also be referred to as C1-C6 alkyl, C₁-C₆ alkyl, or C₁₋₄ alkyl) is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It should also be noted that any carbon as well as heteroatom with unsatisfied valences in the text, schemes, examples and tables herein is assumed to have the sufficient number of hydrogen atom(s) to satisfy the valences.

As used herein, references to hydrogen may also refer to a deuterium substitution if desired. The term “deuterium” as used herein means a stable isotope of hydrogen having odd numbers of protons and neutrons.

Compounds of the various Formulae and pharmaceutically acceptable salts thereof may exist in their tautomeric form (for example, as an amide or imino ether). All such tautomeric forms are contemplated herein as part of the present disclosure.

It should be understood that all isomeric forms are included within the present disclosure, including mixtures thereof. If the compound contains a double bond, the substituent may be in the E or Z configuration. If the compound contains a disubstituted cycloalkyl, the cycloalkyl substituent may have a cis- or trans- configuration.

The compounds of the various Formulae may contain asymmetric or chiral centers, and, therefore, exist in different stereoisomeric forms. It is intended that all stereoisomeric forms of the compounds of the various Formulae as well as mixtures thereof, including racemic mixtures, form part of the present disclosure. In some embodiments, isomers of the compounds herein are stereoisomers. In addition, the present disclosure embraces all geometric and positional isomers.

For example, if a compound of the various Formulae incorporates a double bond or a fused ring, both the cis- and trans-forms, as well as mixtures, are embraced within the scope of the present disclosure. Each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers, and converting (e.g., hydrolyzing) the individual diastereomers to the corresponding pure enantiomers. Also, some of the compounds of the various Formulae may be atropisomers (e.g., substituted biaryls) and are considered as part of the present disclosure. Enantiomers can also be separated by use of a chiral HPLC column.

In some embodiments, the compounds of Formulae I′, II″, I, II, IIa, IIb, IIc, III, IIIa, IIIb, IIIc, IV, IVa, IVb, IVc, V, Va, Vb, VI, VIa, VIb, and VII or pharmaceutically acceptable salts thereof, are enantiomers. In some embodiments, the compounds or pharmaceutically acceptable salts thereof are the (S)-enantiomer. In other embodiments the compounds or pharmaceutically acceptable salts thereof are the (R)-enantiomer. In some embodiments, the compounds or pharmaceutically acceptable salts thereof are the (+) enantiomer or (−) enantiomer.

Some embodiments are directed to isotopically-labelled compounds of the present disclosure which are identical to those recited herein but for the fact that one or more atoms are replaced by an atom having an atomic mass or mass number different from the atomic mass or mass number usually found in nature. Examples of isotopes that can be incorporated into compounds of the present disclosure include isotopes of hydrogen, carbon, nitrogen, oxygen, phosphorus, fluorine and chlorine, such as ²H (or D), ³H, ¹³C, ¹⁴C, ¹⁵N, ¹⁸O, ¹⁷O, ³¹P, ³²p, ³⁵S, ¹⁸F, and ³⁶Cl, respectively.

Certain isotopically-labelled compounds of the various Formulae (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds of the various Formulae can generally be prepared by following procedures analogous to those disclosed in the Schemes and/or in the Examples herein below by substituting an appropriate isotopically labelled reagent for a non-isotopically labelled reagent.

In some embodiments, the compound comprises at least one deuterium atom. For example, one or more hydrogen atoms in a compound of the present disclosure can be replaced or substituted by deuterium. In some embodiments, the compound comprises two or more deuterium atoms. In some embodiments, the compound comprises 1, 2, 3, 4, 5, 6, 7, 8, 9, 10, 11 or 12 deuterium atoms.

The compounds of Formulae I′, II″, I, II, IIa, IIb, IIc, III, IIIa, IIIb, IIIc, IV, IVa, IVb, IVc, V, Va, Vb, VI, VIa, VIb, and VII may form salts which are also within the scope of the present disclosure. Reference to a compound of the Formula herein is understood to include reference to salts thereof, unless otherwise indicated.

The present disclosure is directed to compounds as described herein and pharmaceutically acceptable salts thereof. The present disclosure is also directed to pharmaceutical compositions comprising one or more compounds as described herein or pharmaceutically acceptable salts thereof.

“Pharmaceutically acceptable”, as used herein, refers to that which is useful in preparing a pharmaceutical composition that is generally safe, non-toxic and not biologically or otherwise undesirable, and includes that which is acceptable for veterinary use as well as human pharmaceutical use. For example, provided herein is a pharmaceutical composition comprising a compound of Formulae I′, II″, I, II, IIa, IIb, IIc, III, IIIa, IIIb, IIIc, IV, IVa, IVb, IVc, V, Va, Vb, VI, VIa, VIb, and VII or a pharmaceutically acceptable salt thereof, and a pharmaceutically acceptable excipient.

“Pharmaceutically acceptable salt”, as used herein, refers to a salt which is generally safe, non-toxic and not biologically or otherwise undesirable, and includes that which is acceptable for veterinary use as well as human pharmaceutical use. Such salts may include acid addition salts and base addition salts. Acid addition salts may be formed with inorganic acid such as, but not limited to, hydrochloric acid, hydrobromic acid, sulfuric acid, nitric acid, phosphoric acid and the like; or an organic acid such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, or undecylenic acid. Salts derived from inorganic bases may include, but are not limited to, sodium, potassium, lithium, ammonium, calcium, magnesium, iron, zinc, copper, manganese, and aluminum salts. Salts derived from organic bases may include, but are not limited to, salts of primary, secondary, or tertiary amines; substituted amines including naturally occurring substituted amines; cyclic amines; ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, or N-ethylpiperidine.

The term “carrier”, as used herein, encompasses carriers, excipients, and diluents and refers to a material, composition, or vehicle such as a liquid or solid filler, diluent, excipient, solvent, or encapsulating material involved in carrying or transporting a pharmaceutical agent from one organ, or portion of the body, to another organ, or portion of the body, of a subject. Excipients should be selected on the basis of compatibility and the release profile properties of the desired dosage form. Exemplary carrier materials include, e.g., binders, suspending agents, disintegration agents, filling agents, surfactants, solubilizers, stabilizers, lubricants, wetting agents, diluents, spray-dried dispersions, and the like.

“Pharmaceutically compatible carrier materials” may include, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Hoover, John E., Remington's Pharmaceutical Sciences, Mack Publishing Co., Easton, Pa. 1975.

“Solvate”, as used herein, refers to a complex of variable stoichiometry formed by a solute and solvent. Such solvents for the purpose of the present disclosure may not interfere with the biological activity of the solute. Examples of suitable solvents include, but are not limited to, water, methanol, ethanol, and acetic acid. Solvates wherein water is the solvent molecule are typically referred to as hydrates. Hydrates include compositions containing stoichiometric amounts of water, as well as compositions containing variable amounts of water.

### Compounds

The present disclosure provides a compound of Formula (I′):

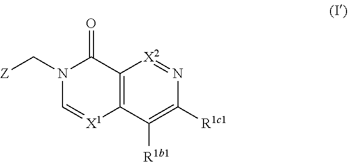

or a pharmaceutically acceptable salt thereof, wherein:
X¹ is CR^{1a} or N;
R^{1a} is selected from the group consisting of H, C₁₋₆alkyl, halo, cycloalkyl, aryl, and heteroaryl; wherein the C₁₋₆alkyl of R^{1a} is unsubstituted or substituted with aryl or heteroaryl, wherein the aryl and heteroaryl is unsubstituted or substituted with one or more substituents selected from the group consisting of of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NR^{1e}C(O)R^{1e};
X² is CH or N;
R^{1b1} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b1} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, —CH(N(R^{1e})₂)COOR^{1e} and —NR^{1e}C(O)R^{1e};
wherein the C₁₋₆alkyl and alkynyl of R^{1b1} is unsubstituted or substituted with one or more substituents selected from the group consisting of halo, aryl, heteroaryl, heterocyclyl, and cycloalkyl;
R^{1c1} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;

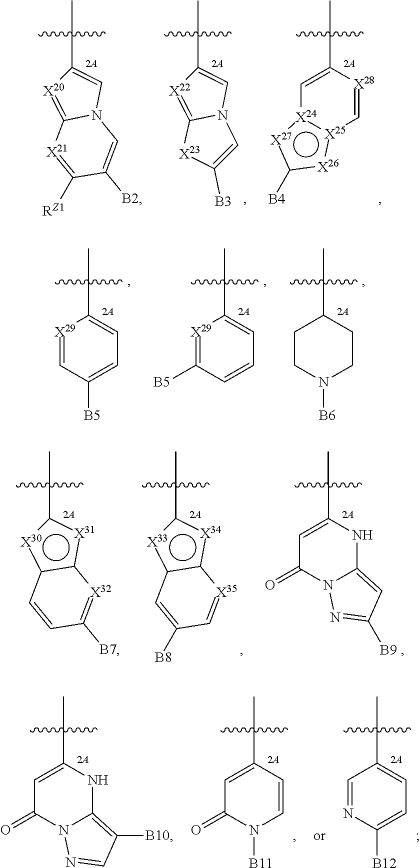

X²⁰ is CR^{20a} or N; wherein R^{20a} is H, halo, CN, aryl, or heteroaryl;
X²¹ is CH or N;
X²² is CR^{22a} or N; wherein R^{22a} is H, halo, CN, aryl, or heteroaryl;
X²³ is CH₂, S, O, NH;
X²⁴ and X²⁵ are independently C or N;
X²⁶ and X²⁷ are independently CH, O, S, N, or NR^{26a}; wherein R^{26a} is H or C₁₋₆alkyl;
X²⁷ is CH or N;
X²⁹ is CH or N;
X³⁰ and X³¹ are independently CH, O, S, N, or NH;
X³² is CH or N;
X³³ and X³⁴ are independently CH, O, S, N, or NH;
X³⁵ is CH or N;
R^{Z1} is H, halo, —CN, or —S(O)₂F;
B2, B3, B4, B7, B8, B9, and B10 are independently selected from the group consisting of H, C₁₋₆alkyl, CN, —S(O)₂F, —CH₂—NR^{2a}R^{2b}

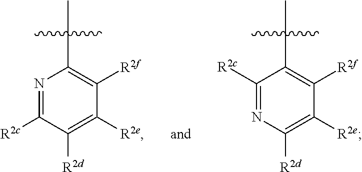

R^{2a} is H or C₁₋₆alkyl;
R^{2b} is selected from the group consisting of H, C₁₋₆alkyl, aryl, heteroaryl, heterocyclyl, —C(NH)-aryl, —S(O)(NH)R^{2g}, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, —S(O)₂F, —S(O)(NH)R^{2g}, alkoxy, aryl, haloaryl, cycloalkyl, halocycloalkyl, hydroxycycloalkyl, and heterocyclyl, wherein the cycloalkyl is unsubstituted or substituted with one or more substituents selected from the group consisting of C₁₋₆alkyl, —(CH₂)ₙF, —(CH₂)ₙBr, or —(CH₂)ₙCN and wherein the heterocyclyl is unsubstituted or substituted with C₁₋₆alkyl; or
R^{2a} and R^{2b} combine to form a 5-7-membered heterocyclyl, wherein the heterocyclyl is unsubstituted or substituted with one or more substituents selected from the group consisting of C₁₋₆alkyl and cycloalkyl;
R^{2c}, R^{2d}, R^{2e}, and R^{2f} are each independently H, C₁₋₆alkyl, heterocyclyl, or —NHR^{2g};
R^{2g} is selected from the group consisting of H, C₁₋₆alkyl, and cycloalkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;
B5, B6, B11, and B12 are independently

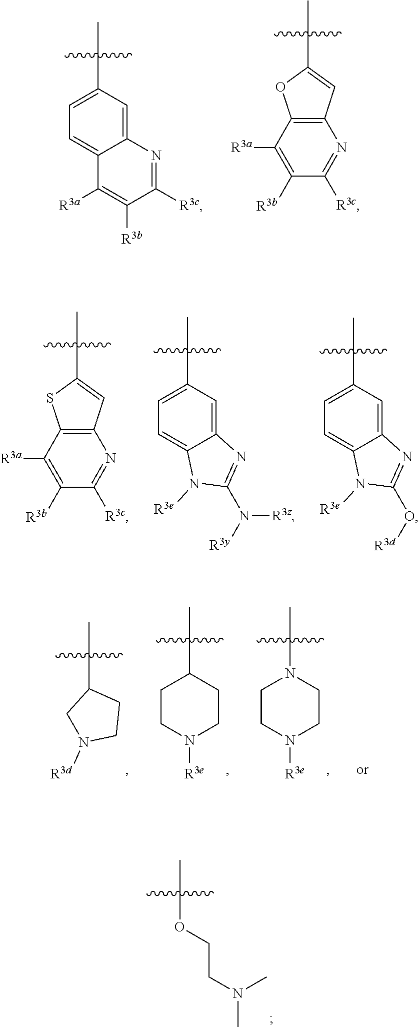

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, —OR^{3x′}, and —CO₂R^{x};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

The present disclosure provides a compound of Formula (I″):

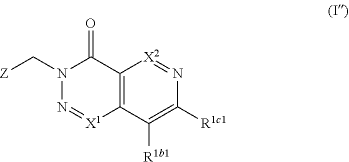

or a pharmaceutically acceptable salt thereof, wherein:
X¹ is CR^{1a} or N;
R^{1a} is selected from the group consisting of H, C₁₋₆alkyl, halo, cycloalkyl, aryl, and heteroaryl; wherein the C₁₋₆alkyl of R^{1a} is unsubstituted or substituted with aryl or heteroaryl, wherein the aryl and heteroaryl is unsubstituted or substituted with one or more substituents selected from the group consisting of of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NR^{1e}C(O)R^{1e};
X² is CH or N;
R^{1b1} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b1} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, —CH(N(R^{1e})₂)COOR^{1e} and —NR^{1e}C(O)R^{1e};
wherein the C₁₋₆alkyl and alkynyl of R^{1b1} is unsubstituted or substituted with one or more substituents selected from the group consisting of halo, aryl, heteroaryl, heterocyclyl, and cycloalkyl;
R^{1c1} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
Z is

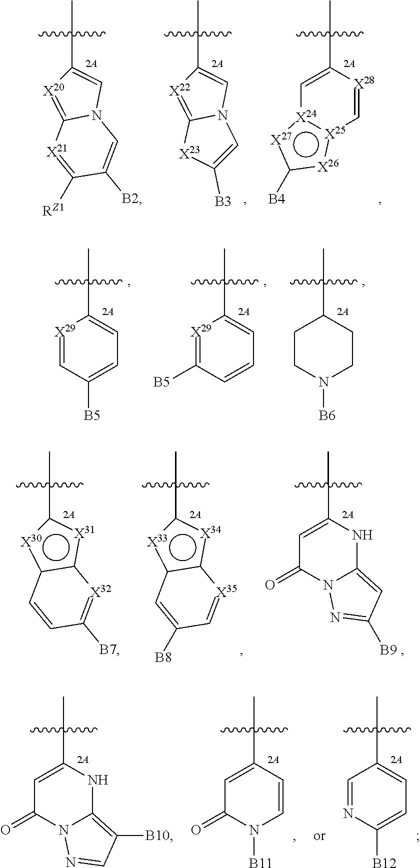

X²⁰ is CR^{20a} or N; wherein R^{20a} is H, halo, CN, aryl, or heteroaryl;
X²¹ is CH or N;
X²² is CR^{22a} or N; wherein R^{22a} is H, halo, CN, aryl, or heteroaryl;
X²³ is CH₂, S, O, NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
X²⁹ is CH or N;
X³⁰ and X³¹ are independently CH, O, S, N, or NH;
X³² is CH or N;
X³³ and X³⁴ are independently CH, O, S, N, or NH;
X³⁵ is CH or N;
R^{Z1} is H, halo, —CN, or —S(O)₂F;
B2, B3, B4, B7, B8, B9, and B10 are independently selected from the group consisting of H, C₁₋₆alkyl, CN, —S(O)₂F, —CH₂—NR^{2a}R^{2b},

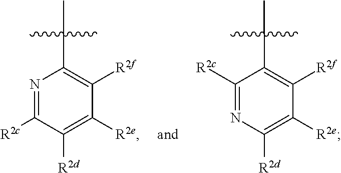

R^{2a} is H or C₁₋₆alkyl;
R^{2b} is selected from the group consisting of H, C₁₋₆alkyl, aryl, heteroaryl, heterocyclyl, —C(NH)-aryl, —S(O)(NH)R^{2g}, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, —S(O)₂F, —S(O)NH)R^{2g}, alkoxy, aryl, haloaryl, cycloalkyl, halocycloalkyl, hydroxycycloalkyl, and heterocyclyl, wherein the cycloalkyl is unsubstituted or substituted with one or more substituents selected from the group consisting of C₁₋₆alkyl, —(CH₂)ₙF, —(CH₂)ₙBr, or —(CH₂)ₙCN and wherein the heterocyclyl is unsubstituted or substituted with C₁₋₆alkyl; or
R^{2a} and R^{2b} combine to form a 5-7-membered heterocyclyl, wherein the heterocyclyl is unsubstituted or substituted with one or more substituents selected from the group consisting of C₁₋₆alkyl and cycloalkyl;
R^{2c}, R^{2d}, R^{2e}, and R^{2f} are each independently H, C₁₋₆alkyl, heterocyclyl, or —NHR^{2g};
R^{2g} is selected from the group consisting of H, C₁₋₆alkyl, and cycloalkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;
B5, B6, B11, and B12 are independently

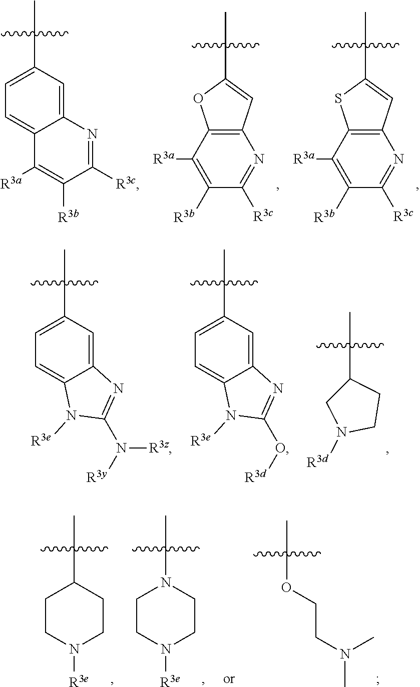

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, —OR^{3x′}, and —CO₂R^{x};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

The present disclosure provides a compound of Formula (I):

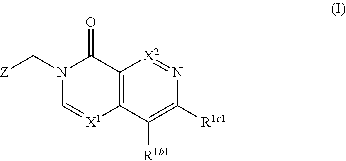

or a pharmaceutically acceptable salt thereof, wherein:
X¹ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X² is CH or N;
R^{1b1} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b1} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b1} is unsubstituted or substituted with one or more substituents selected from the group consisting of aryl, heterocyclyl, and cycloalkyl;
R^{1c1} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
Z is

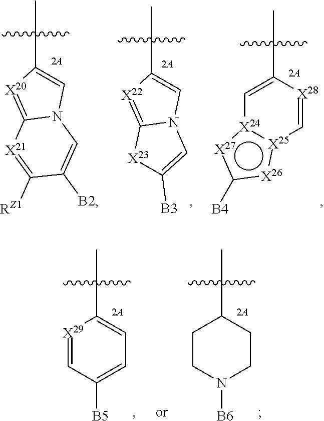

X²⁰ is CR^{20a} or N; wherein R^{20a} is H, halo, CN, aryl, or heteroaryl;
X²¹ is CH or N;
X²² is CR^{22a} or N; wherein R^{22a} is H, halo, CN, aryl, or heteroaryl;
X²³ is CH₂, S, O, NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
X²⁹ is CH or N;
B2, B3, and B4 are independently selected from the group consisting of H, C₁₋₆alkyl, CN, —CH₂—NR^{2a}R^{2b},

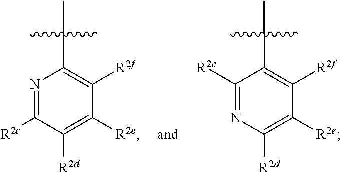

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;
B5 and B6 are independently

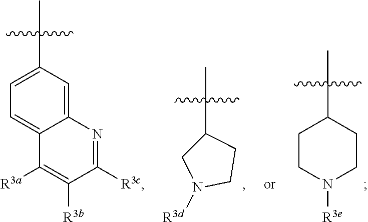

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

In some embodiments of the compounds of Formulae (I′) and (I) or the pharmaceutically acceptable salts thereof,
X¹ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X² is CH or N;
R^{1b1} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b1} is unsubstituted or substituted with one or more substituents selected from the group consisting of C₁₋₆alkyl, halo, —CN, —(CH₂)ₘNH₂, —(CH₂)ₘOH, —C(O)Re, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b1} is unsubstituted or substituted with one or more substituents selected from the group consisting of aryl, heterocyclyl, and cycloalkyl;
R^{1c1} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl; wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
Z is

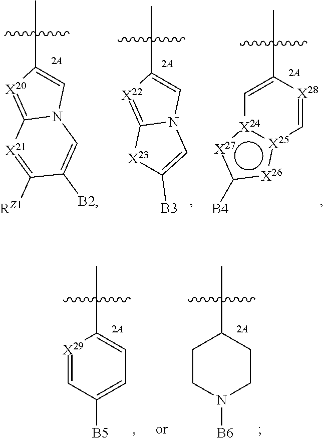

X²⁰ is CR^{20a} or N; wherein R^{20a} is H, halo, CN, aryl, or heteroaryl;
X²¹ is CH or N;
X²² is CR^{22a} or N; wherein R^{22a} is H, halo, CN, aryl, or heteroaryl;
X²³ is CH₂, S, O, NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
X²⁹ is CH or N;
B2, B3, and B4 are independently selected from the group consisting of H, C₁₋₆alkyl, CN, —CH₂—NR^{2a}R^{2b},

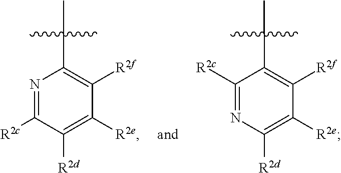

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;
B5 and B6 are independently

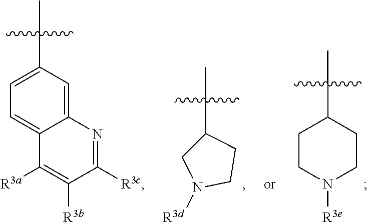

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

In some embodiments of the compounds of Formulae (I′) and (I) or the pharmaceutically acceptable salts thereof, X¹ is CR^{1a}. In some embodiments, R^{1a} is H. In some embodiments, R^{1a} is C₁₋₆alkyl, wherein the C₁₋₆alkyl is unsubstituted or substituted with aryl. In some embodiments, R^{1a} is halo, cycloalkyl, or heteroaryl. In some embodiments, R^{1a} is aryl. In some embodiments, R^{1a} is C₁₋₆alkyl, wherein the C₁₋₆alkyl of R^{1a} is unsubstituted or substituted with aryl or heteroaryl, wherein the aryl and heteroaryl is unsubstituted or substituted with one or more substituents selected from the group consisting of of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NR^{1e}C(O)R^{1e}.

In some embodiments of the compounds of Formulae (I′) and (I) or the pharmaceutically acceptable salts thereof, X¹ is N.

In some embodiments of the compounds of Formulae (I′) and (I) or the pharmaceutically acceptable salts thereof, X² is CH. In some embodiments, X¹ is CR^{1a} and X² is CH.

In some embodiments of the compounds of Formulae (I′) and (I) or the pharmaceutically acceptable salts thereof, X² is N.

In some embodiments of the compounds of Formulae (I′) and (I) or the pharmaceutically acceptable salts thereof, R^{1c1} is H. In some embodiments, X¹ is CR^{1a}, X² is CH, and R^{1c1} is H.

In some embodiments of the compounds of Formulae (I′) and (I) or the pharmaceutically acceptable salts thereof, R^{1c1} is C₁₋₆alkyl.

In some embodiments of the compounds of Formulae (I′) and (I) or the pharmaceutically acceptable salts thereof, R^{1c1} is halo, —CN, —N(R^{1d})₂, or —OR^{1d}. In some embodiments, R^{1d} is H or C₁₋₆alkyl. In some embodiments, R^{1d} is —(CH₂)ₘCN or —(CH₂)ₘNH₂. In some embodiments, R^{1d} is cycloalkyl or heterocyclyl.

In some embodiments of the compounds of Formulae (I′) and (I) or the pharmaceutically acceptable salts thereof, R^{1b1} is aryl, wherein the aryl is unsubstituted or substituted with one or more substituents selected from the group consisting of C₁₋₆alkyl, halo, —CN, —(CH₂)nOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the aryl is unsubstituted. In some embodiments, the aryl is phenyl.

In some embodiments of the compounds of Formulae (I′) and (I) or the pharmaceutically acceptable salts thereof, R^{1b1} is heteroaryl or heterocyclyl, wherein the heteroaryl and heterocyclyl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the heteroaryl or heterocyclyl is unsubstituted. In some embodiments, R^{1b1} is heteroaryl substituted with —NHC(O)R^{1e}. In some embodiments, R^{1b1} is

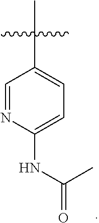 [SMILES: *c1ccc(NC(C)=O)nc1]

In some embodiments, R^{1b1} is heterocyclyl wherein the heterocyclyl is unsubstituted.
In some embodiments, R^{1b1} is

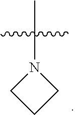 [SMILES: *N1CCC1]

In some embodiments of the compounds of Formulae (I′) and (I) or the pharmaceutically acceptable salts thereof, R^{1b1} is cycloalkyl or cycloalkenyl, wherein the cycloalkyl and cycloalkenyl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the cycloalkyl or cycloalkenyl is unsubstituted.

In some embodiments of the compounds of Formulae (I′) and (I) or the pharmaceutically acceptable salts thereof, R^{1b1} is C₁₋₆alkyl or alkynyl, wherein the C₁₋₆alkyl and alkynyl is unsubstituted or substituted with one or more substituents selected from the group consisting of aryl, heterocyclyl, and cycloalkyl. In some embodiments, the C₁₋₆alkyl is substituted with heterocyclyl. In some embodiments, the C₁₋₆alkyl is substituted with cycloalkyl.

In some embodiments of the compounds of Formulae (I′) and (I) or the pharmaceutically acceptable salts thereof, R^{1b1} is H.

In some embodiments of the compounds of Formulae (I′) and (I) or the pharmaceutically acceptable salts thereof, R^{1b1} is halo.

In some embodiments of the compounds of Formulae (I′) and (I) or the pharmaceutically acceptable salts thereof, R^{1b1} is —OR^{1e} or —N(R^{1e})₂. In some embodiments, R^{1e} is C₁₋₆alkyl. In some embodiments, R^{1e} is —(CH₂)ₘCN. In some embodiments, R^{1e} is —(CH₂)ₘNH₂. In some embodiments, R^{1e} is cycloalkyl. In some embodiments, R^{1e} is heterocyclyl.

As described herein, Z is

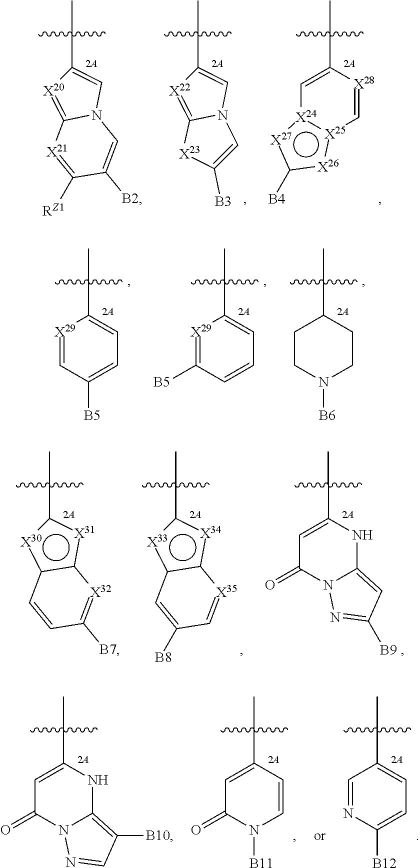

In some embodiments of the compounds of Formulae (I′) and (I) or the pharmaceutically acceptable salts thereof, X²⁰ is CR^{20a}. In some embodiments, X²⁰ is CR^{20a} wherein R^{20a} is H. In some embodiments, X²⁰ is CH. In some embodiments, X²⁰ is CR^{20a} wherein R^{20a} is halo, CN, aryl, or heteroaryl.

In some embodiments of the compounds of Formulae (I′) and (I) or the pharmaceutically acceptable salts thereof, X²⁰ is N.

In some embodiments of the compounds of Formulae (I′) and (I) or the pharmaceutically acceptable salts thereof, X²¹ is CH.

In some embodiments of the compounds of Formulae (I′) and (I) or the pharmaceutically acceptable salts thereof, X²¹ is N.

In some embodiments of the compounds of Formulae (I′) and (I) or the pharmaceutically acceptable salts thereof, X²⁰ is N and X²¹ is CH.

In some embodiments of the compounds of Formulae (I′) and (I) or the pharmaceutically acceptable salts thereof, X²² is CR^{22a}. In some embodiments, X²² is CR^{22a} wherein R^{22a} is H. In some embodiments, X²² is CH. In some embodiments, X²² is CR^{2a} wherein R^{22a} is halo or aryl.

In some embodiments of the compounds of Formulae (I′) and (I) or the pharmaceutically acceptable salts thereof, X²² is N.

In some embodiments of the compounds of Formulae (I′) and (I) or the pharmaceutically acceptable salts thereof, X²³ is CH₂.

In some embodiments of the compounds of Formulae (I′) and (I) or the pharmaceutically acceptable salts thereof, X²³ is NH.

In some embodiments of the compounds of Formulae (I′) and (I) or the pharmaceutically acceptable salts thereof, X²³ is S or O.

In some embodiments of the compounds of Formulae (I′) and (I) or the pharmaceutically acceptable salts thereof, X²² is N and X²³ is S.

In some embodiments of the compounds of Formulae (I′) and (I) or the pharmaceutically acceptable salts thereof, X²⁴ and X²⁵ are C.

In some embodiments of the compounds of Formulae (I′) and (I) or the pharmaceutically acceptable salts thereof, X²⁴ is N and X²⁵ is C.

In some embodiments of the compounds of Formulae (I′) and (I) or the pharmaceutically acceptable salts thereof, X²⁶ and X²⁷ are independently N or NR^{26a} wherein R^{26a} is H or C₁₋₆alkyl. In some embodiments, X²⁶ and X²⁷ are independently N or NCH₃. In some embodiments, X²⁶ and X²⁷ are independently N or NH.

In some embodiments of the compounds of Formulae (I′) and (I) or the pharmaceutically acceptable salts thereof, X²⁶ and X²⁷ are CH.

In some embodiments of the compounds of Formulae (I′) and (I) or the pharmaceutically acceptable salts thereof, X²⁸ is CH.

In some embodiments of the compounds of Formulae (I′) and (I) or the pharmaceutically acceptable salts thereof, X²⁸ is N.

In some embodiments of the compounds of Formulae (I′) and (I) or the pharmaceutically acceptable salts thereof, X²⁴ and X²⁵ are C and X²⁸ is CH.

In some embodiments of the compounds of Formulae (I′) and (I) or the pharmaceutically acceptable salts thereof, X²⁶ and X²⁷ are independently N or NR²⁶ wherein R²⁶ is H or C₁₋₆alkyl, and X²⁸ is CH.

In some embodiments of the compounds of Formulae (I′) and (I) or the pharmaceutically acceptable salts thereof, X²⁴ and X²⁵ are C, X²⁶ and X²⁷ are independently N or NR^{26a} wherein R^{26a} is H or C₁₋₆alkyl, and X²⁸ is CH.

In some embodiments of the compounds of Formulae (I′) and (I) or the pharmaceutically acceptable salts thereof, B2, B3, or B4 is —CH₂—NR^{2a}R^{2b}.

In some embodiments of the compounds of Formulae (I′) and (I) or the pharmaceutically acceptable salts thereof, R^{2b} is C₁₋₆alkyl, wherein the C₁₋₆alkyl is unsubstituted or substituted with one or more substituents selected from the group consisting of C₁₋₆alkyl, OH, alkoxy, aryl, haloaryl, cycloalkyl, halocycloalkyl and heterocyclyl; wherein the heterocyclyl is unsubstituted or substituted with C₁₋₆alkyl. In some embodiments, R^{2b} is C₁₋₆alkyl, wherein the C₁₋₆alkyl is unsubstituted. In some embodiments, R^{2b} is —CH₃, —CH₂CH₃, or

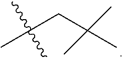 [SMILES: *CC(C)(C)C]

In some embodiments, R^{2b} is C₁₋₆alkyl, wherein the C₁₋₆alkyl is substituted with one or more substituents selected from the group consisting of cycloalkyl and halocycloalkyl. In some embodiments R^{2b} is

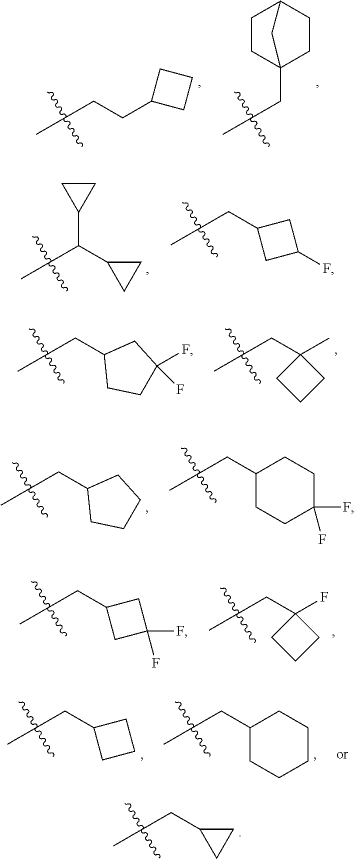

In some embodiments, R^{2b} is

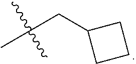 [SMILES: *CC1CCC1]

In some embodiments, R^{2b} is C₁₋₆alkyl, wherein the C₁₋₆alkyl is substituted with one or more heterocyclyl. In some embodiments, R^{2b} is

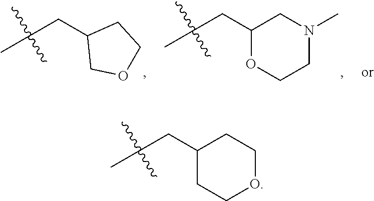

In some embodiments, R^{2b} is C₁₋₆alkyl, wherein the C₁₋₆alkyl is substituted with one or more substituents selected from the group consisting of cycloalkyl, aryl, and haloaryl. In some embodiments, R^{2b} is

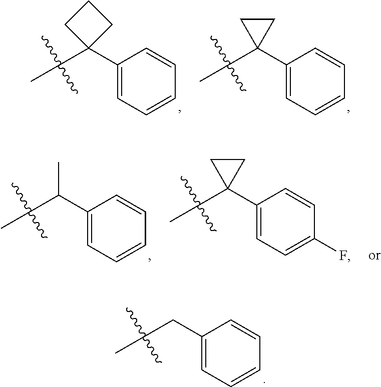

In some embodiments, R^{2b} is C₁₋₆alkyl, wherein the C₁₋₆alkyl is substituted with one or more substituents selected from the group consisting of cycloalkyl and OH. In some embodiments, R^{2b} is

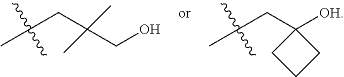

In some embodiments of the compounds of Formulae (I′) and (I) or the pharmaceutically acceptable salts thereof, R^{2b} is cycloalkyl, wherein the cycloalkyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is cycloalkyl wherein the cycloalkyl is unsubstituted. In some embodiments, R^{2b} is

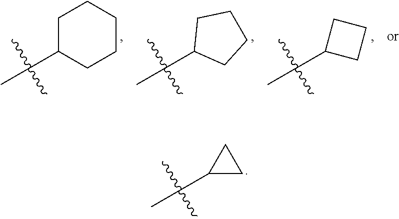

In some embodiments of the compounds of Formulae (I′) and (I) or the pharmaceutically acceptable salts thereof, R^{2b} is aryl, wherein the aryl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is aryl and is

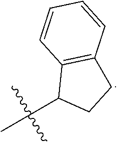 [SMILES: *C1CCc2ccccc21]

In some embodiments of the compounds of Formulae (I′) and (I) or the pharmaceutically acceptable salts thereof, R^{2b} is selected from the group consisting of heteroaryl and heterocyclyl, wherein the heteroaryl and heterocyclyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is selected from the group consisting of heteroaryl and heterocyclyl wherein the heteroaryl and heterocyclyl is unsubstituted. In some embodiments, R^{2b} is selected from the group consisting of heteroaryl and heterocyclyl, wherein the heteroaryl and heterocyclyl is substituted with one or more C₁₋₆alkyl. In some embodiments, R^{2b} is

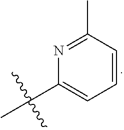 [SMILES: *c1cccc(C)n1]

In some embodiments, R^{2b} is selected from the group consisting of heteroaryl and heterocyclyl, wherein the heteroaryl and heterocyclyl is substituted with one or more alkoxy. In some embodiments, R^{2b} is

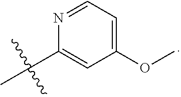 [SMILES: *c1cc(OC)ccn1]

In some embodiments, R^{2b} is selected from the group consisting of heteroaryl and heterocyclyl, wherein the heteroaryl and heterocyclyl is substituted with one or more heterocyclyl that is unsubstituted or substituted with C1-6alkyl. In some embodiments, R^{2b} is

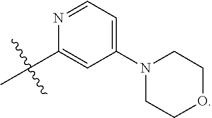 [SMILES: *c1cc(N2CCOCC2)ccn1]

In some embodiments of the compounds of Formulae (I′) and (I) or the pharmaceutically acceptable salts thereof, R^{2b} is selected from the group consisting of —CH₃, —CH₂CH₃,

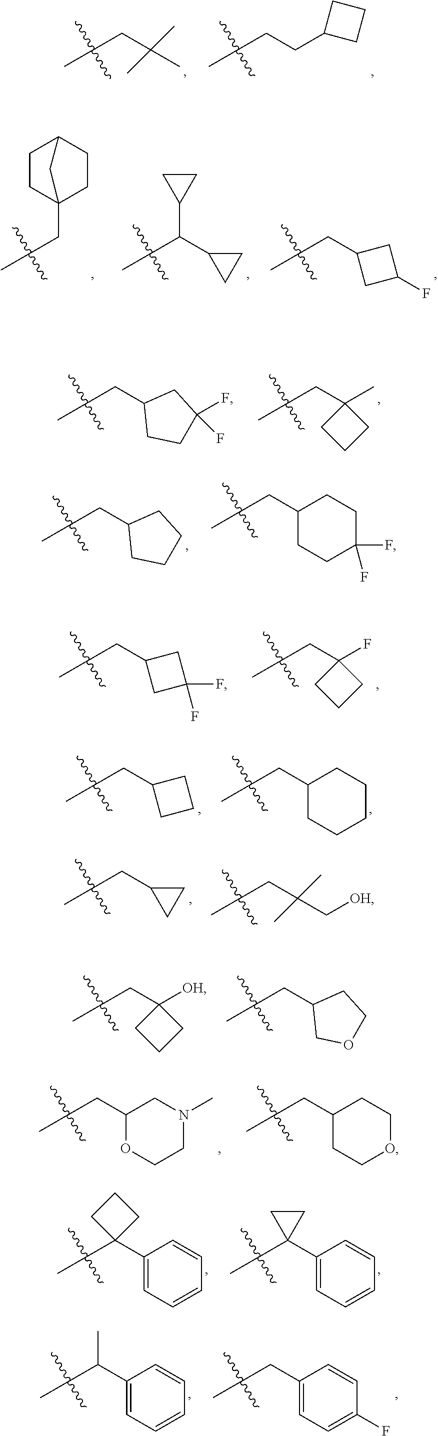

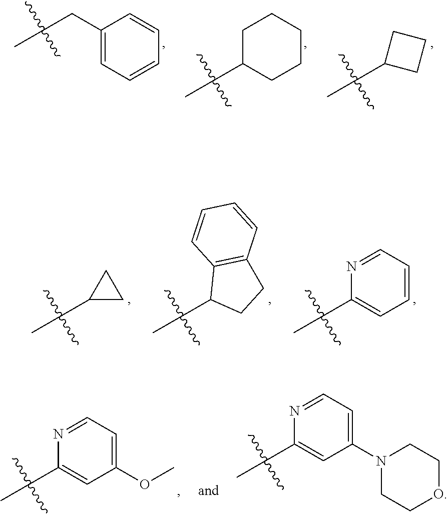

In some embodiments, R^{2b} is

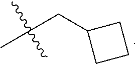 [SMILES: *CC1CCC1]

In some embodiments of the compounds of Formulae (I′) and (I) or the pharmaceutically acceptable salts thereof, R^{2a} is H. In some embodiments, R^{2a} is H and R^{2b} is selected from the group consisting of —CH₃, —CH₂CH₃,

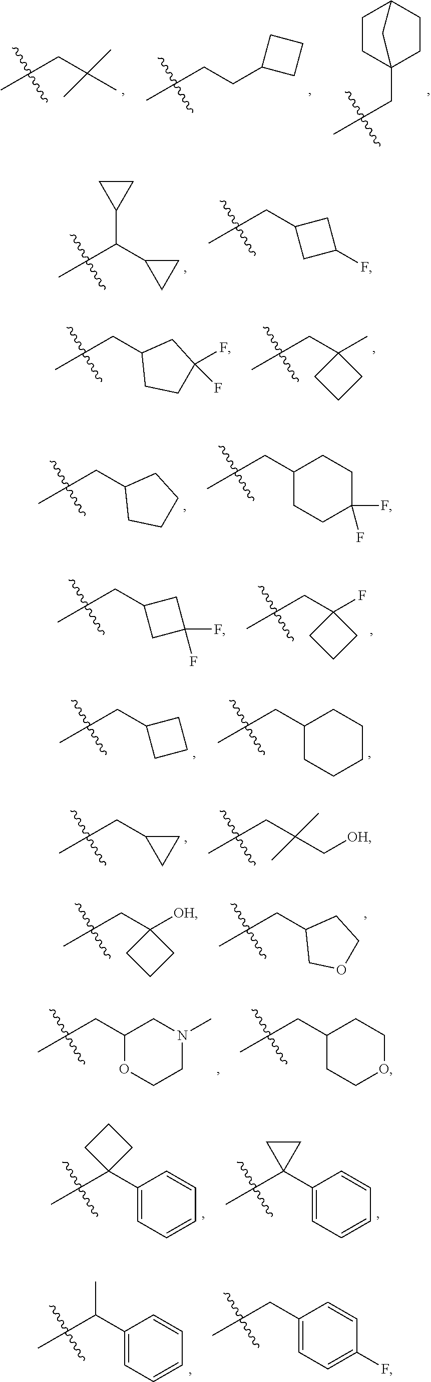

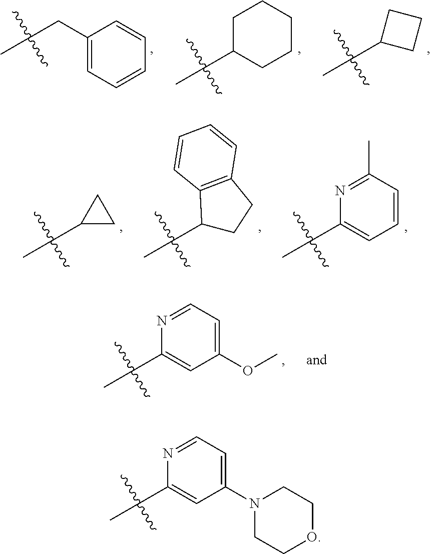

In some embodiments, R^{2a} is H and R^{2b} is

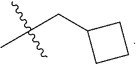 [SMILES: *CC1CCC1]

In some embodiments of the compounds of Formulae (I′) and (I) or the pharmaceutically acceptable salts thereof, R^{2a} and R^{2b} combine to form a 5-7-membered heterocyclyl, wherein the heterocyclyl is substituted with C₁₋₆alkyl. In some embodiments, R^{2a} and R^{2b} combine to form

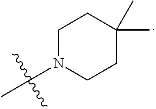 [SMILES: *N1CCC(C)(C)CC1]

In some embodiments of the compounds of Formulae (I′) and (I) or the pharmaceutically acceptable salts thereof, R^{2a} and R^{2b} combine to form a 5-7-membered heterocyclyl, wherein the heterocyclyl is unsubstituted.

In some embodiments of the compounds of Formulae (I′) and (I) or the pharmaceutically acceptable salts thereof, B2, B3, or B4 is

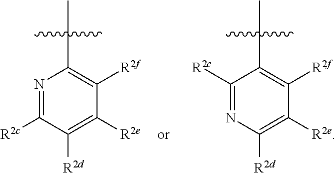

In some embodiments, one of R^{2c}, R^{2d}, R^{2e}, and R^{2f} is heterocyclyl and the others are H. In some embodiments, one of R^{2c}, R^{2d}, R^{2e}, and R^{2f} is

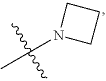 [SMILES: *N1CCC1]

and the others are H. In some embodiments, one of R^{2c}, R^{2d}, R^{2e}, and R^{2f} is —NHR^{2g} and the others are H. In some embodiments, R^{2g} is C₁₋₆alkyl, wherein the C₁₋₆alkyl is unsubstituted or substituted with cycloalkyl. In some embodiments, R^{2g} is —CH₃, —CH₂CH₃, or

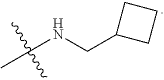 [SMILES: *NCC1CCC1]

In some embodiments R^{2g} is H. In some embodiments, one of R^{2c}, R^{2d}, R^{2e}, and R^{2f} is C₁₋₆alkyl or heterocyclyl and the others are H.

In some embodiments of the compounds of Formulae (I′) and (I) or the pharmaceutically acceptable salts thereof, B2, B3, or B4 is C₁₋₆alkyl. In some embodiments, B2, B3, or B4 is —CH³.

In some embodiments of the compounds of Formulae (I′) and (I) or the pharmaceutically acceptable salts thereof, B2, B3, or B4 is H or CN.

In some embodiments of the compounds of Formulae (I′) and (I) or the pharmaceutically acceptable salts thereof, X²⁹ is CH.

In some embodiments of the compounds of Formulae (I′) and (I) or the pharmaceutically acceptable salts thereof, X²⁹ is N.

In some embodiments of the compounds of Formulae (I′) and (I) or the pharmaceutically acceptable salts thereof, B5 or B6 is

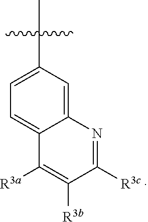

In some embodiments, one or two of R^{3a}, R^{3b}, and R^{3c} is —OR^{3x}. In some embodiments, R^{3x} is C₁₋₆alkyl. In some embodiments, one or two of R^{3a}, R^{3b}, and R^{3c} is —OCH₃. In some embodiments, one of R^{3a}, R^{3b}, and R^{3c} is —OR^{3x} and the others of R^{3a}, R^{3b}, and R^{3c} are H.

In some embodiments, one or two of R^{3a}, R^{3b}, and R^{3c} is —NR^{3y}R^{3z}. In some embodiments, one or both of R^{3y} and R^{3z} is H. In some embodiments, one or both of R^{3y} and R^{3z} is C₁₋₆alkyl, wherein C₁₋₆alkyl is unsubstituted or substituted with one or more substituents selected from the group consisting of cycloalkyl and —OCH₃. In some embodiments, one or both of R^{3y} and R^{3z} is —CH₃, —CH₂CH₃,

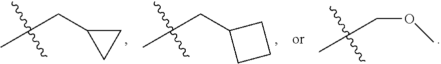

In some embodiments, one of R^{3y} and R^{3z} is cycloalkyl. In some embodiments, one of R^{3y} and R^{3z} is

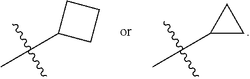

In some embodiments, both R^{3y} and R^{3z} are C₁₋₆alkyl, wherein C₁₋₆alkyl is unsubstituted. In some embodiments, one or two of R^{3a}, R^{3b}, and R^{3c} is —N(CH₃)₂. In some embodiments, one of R^{3a}, R^{3b}, and R^{3c} is —NR^{3y}R^{3z} and the others of R^{3a}, R^{3b}, and R^{3c} are H.

In some embodiments, one or two of R^{3a}, R^{3b}, and R^{3c} is heterocyclyl wherein the heterocyclyl is unsubstituted or substituted with one or more substituents selected from the group consisting of C₁₋₆alkyl, halogen, and —OH. In some embodiments, one or two of R^{3a}, R^{3b}, and R^{3c} is

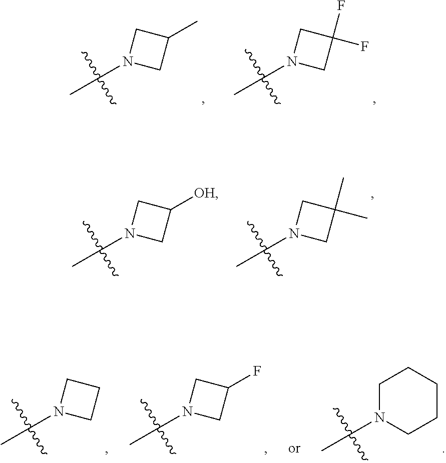

In some embodiments, one of R^{3a}, R^{3b}, and R^{3c} is heterocyclyl wherein the heterocyclyl is unsubstituted or substituted with one or more substituents selected from the group consisting of C₁₋₆alkyl, halogen, and —OH, and the others of R^{3a}, R^{3b}, and R^{3c} are H.

In some embodiments, two of R^{3a}, R^{3b}, and R^{3c} are each independently C₁₋₆alkyl, —OR^{3x}, —NR^{3y}R^{3z}, cycloalkyl, or heterocyclyl, and the other of R^{3a}, R^{3b}, and R^{3c} is H.

In some embodiments of the compounds of Formulae (I′) and (I) or the pharmaceutically acceptable salts thereof, B5 or B6 is

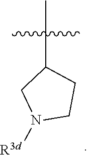

In some embodiments, R^{3d} is C₁₋₆alkyl, wherein C₁₋₆alkyl is unsubstituted or substituted with cycloalkyl.

In some embodiments of the compounds of Formulae (I′) and (I) or the pharmaceutically acceptable salts thereof, B5 or B6 is

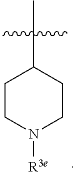

In some embodiments, R^{3e} is C₁₋₆alkyl, wherein C₁₋₆alkyl is unsubstituted or substituted with cycloalkyl.

The present disclosure also provides a compound of Formula (II), (III), (IV), (V), or (VI):

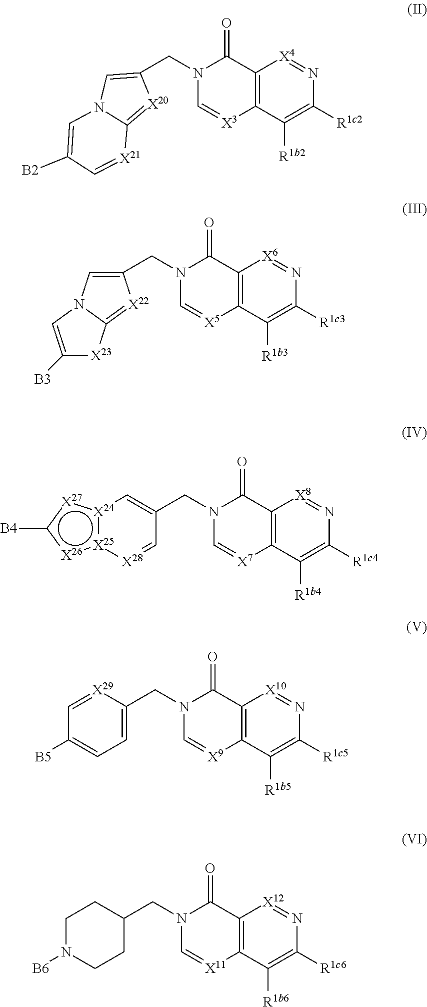

or a pharmaceutically acceptable salt thereof, wherein:
X³, X⁵, X⁷, X⁹, and X¹¹ are independently CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X⁴, X⁶, X⁸, X¹⁰, and X¹² are independently CH or N;
R^{1b2}, R^{1b3}, R^{1b4}, R^{1b5}, and R^{1b6} are independently selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b2}, R^{1b3}, R^{1b4}, R^{1b5}, and R^{1b6} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b2}, R^{1b3}, R^{1b4}, R^{1b5}, and R^{1b6} is unsubstituted or substituted with one or more substituents selected from the group consisting of aryl, heterocyclyl, and cycloalkyl;
R^{1c2}, R^{1c3}, and R^{1c4} are independently selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁰ is CR^{20a} or N; wherein R^{20a} is H, halo, CN, aryl, or heteroaryl;
X²¹ is CH or N;
X²² is CR^{22a} or N; wherein R^{22a} is H, halo, aryl, or heteroaryl;
X²³ is CH, S, O, or NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
B2, B3, and B4 are independently selected from the group consisting of H, C₁₋₆alkyl, CN, —CH₂—NR^{2a}R^{2b},

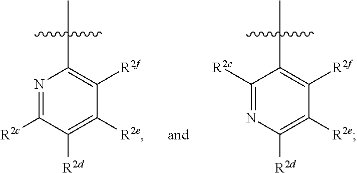

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;
X²⁹ is CH or N;
B5 and B6 are independently

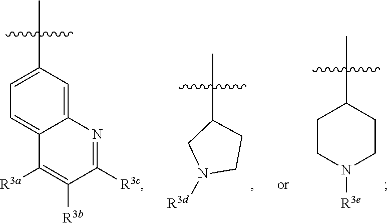

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted cycloalkyl; and
R^{3e} is selected from the group consisting of H and C₁₋₆alkyl; wherein C₁₋₆alkyl is unsubstituted or substituted cycloalkyl.

In some embodiments, the compound is of Formula (II):

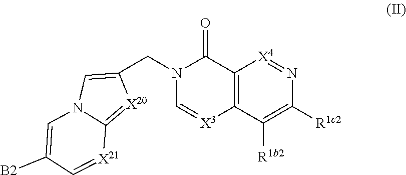

or a pharmaceutically acceptable salt thereof, wherein:
X³ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X⁴ is CH or N;
R^{1b2} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b2} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b2} is unsubstituted or substituted with one or more substituents selected from the group consisting of aryl, heterocyclyl, and cycloalkyl;
R^{1c2} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁰ is CR^{20a} or N; wherein R^{20a} is H, halo, CN, aryl, or heteroaryl;
X²¹ is CH or N;
B2 is selected from the group consisting of H, C₁₋₆alkyl, CN, —CH₂—NR^{2a}R^{2b},

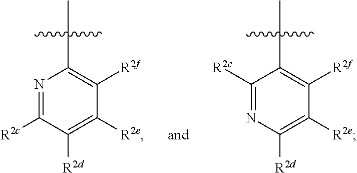

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

In some embodiments of the compounds of Formula (II) or the pharmaceutically acceptable salts thereof, X³ is CR^{1a}. In some embodiments, R^{1a} is H. In some embodiments, R^{1a} is C₁₋₆alkyl, wherein the C₁₋₆alkyl is unsubstituted or substituted with aryl. In some embodiments, R^{1a} is halo, cycloalkyl, or heteroaryl.

In some embodiments of the compounds of Formula (II) or the pharmaceutically acceptable salts thereof, X³ is N.

In some embodiments of the compounds of Formula (II) or the pharmaceutically acceptable salts thereof, X⁴ is CH. In some embodiments, X³ is CR^{1a} and X⁴ is CH.

In some embodiments of the compounds of Formula (II) or the pharmaceutically acceptable salts thereof, X⁴ is N.

In some embodiments, the compound of Formula (II), or the pharmaceutically acceptable salt thereof, is a compound of Formula (IIa), or a pharmaceutically acceptable salt thereof:

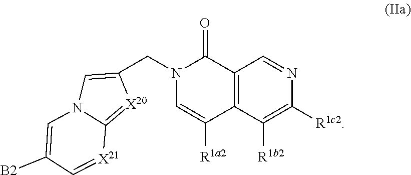

In some embodiments, the compound of Formula (II), or the pharmaceutically acceptable salt thereof, is a compound of Formula (IIb), or a pharmaceutically acceptable salt thereof:

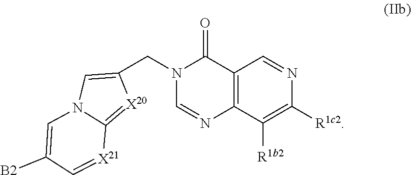

In some embodiments of the compounds of Formulae (II), (IIa), and (IIb), or the pharmaceutically acceptable salts thereof, B2 is —CH₂—NR^{2a}R^{2b}.

In some embodiments of the compounds of Formulae (II), (IIa), and (IIb) or the pharmaceutically acceptable salts thereof, R^{2a} is H. In some embodiments, R^{2a} is H and R^{2b} is selected from the group consisting of —CH₃, —CH₂CH₃,

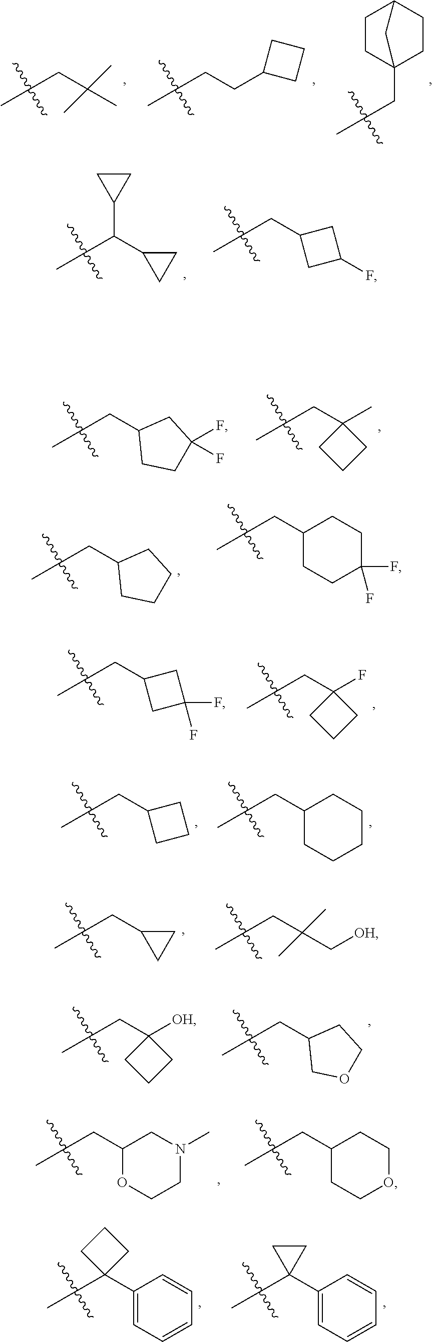

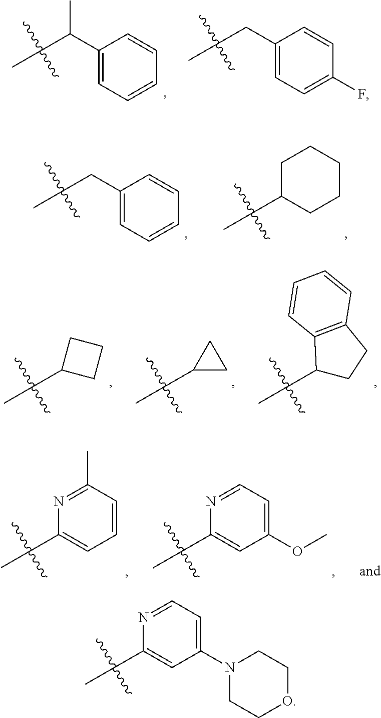

In some embodiments, R^{2a} is H and R^{2b} is

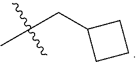 [SMILES: *CC1CCC1]

In some embodiments of the compounds of Formulae (II), (IIa), and (IIb), or the pharmaceutically acceptable salts thereof, R^{2a} and R^{2b} combine to form a 5-7-membered heterocyclyl, wherein the heterocyclyl is substituted with C₁₋₆alkyl. In some embodiments, R² and R^{2b} combine to form

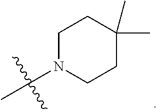 [SMILES: *N1CCC(C)(C)CC1]

In some embodiments of the compounds of Formulae (II), (IIa), and (IIb), or the pharmaceutically acceptable salts thereof, R^{2a} and R^{2b} combine to form a 5-7-membered heterocyclyl, wherein the heterocyclyl is unsubstituted.

In some embodiments of the compounds of Formulae (II), (IIa), and (IIb), or the pharmaceutically acceptable salts thereof, B2 is

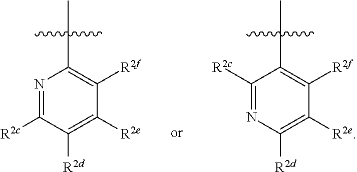

In some embodiments, one of R^{2c}, R^{2d}, R^{2e}, and R^{2f} is heterocyclyl and the others are H. In some embodiments, one of R^{2c}, R^{2d}, R^{2e}, and R^{2f} is

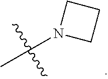 [SMILES: *N1CCC1]

and the others are H. In some embodiments, one of R^{2c}, R^{2d}, R^{2e}, and R^{2f} is —NHR^{2g} and the others are H. In some embodiments, R^{2g} is C₁₋₆alkyl, wherein the C₁₋₆alkyl is unsubstituted or substituted with cycloalkyl. In some embodiments, R^{2g} is —CH₃, —CH₂CH₃, or

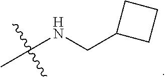 [SMILES: *NCC1CCC1]

In some embodiments R^{2g} is H. In some embodiments, one of R^{2c}, R^{2d}, R^{2e}, and R^{2f} is C₁₋₆alkyl or heterocyclyl and the others are H.

In some embodiments of the compounds of Formulae (II), (IIa), and (IIb), or the pharmaceutically acceptable salts thereof, B2 is C₁₋₆alkyl. In some embodiments, B2 is —CH³.

In some embodiments of the compounds of Formulae (II), (IIa), and (IIb) or the pharmaceutically acceptable salts thereof, B2 is H or CN.

In some embodiments, the compound of Formula (II), or the pharmaceutically acceptable salt thereof, is a compound of Formula (IIc), or a pharmaceutically acceptable salt thereof:

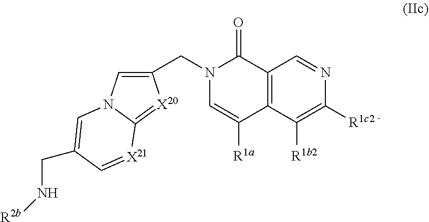

In some embodiments of the compounds of Formulae (II), (IIa), (IIb), and (IIc), or the pharmaceutically acceptable salts thereof, R^{1c2} is H. In some embodiments, X³ is CR^{1a}, X⁴ is CH, and R^{1c2} is H.

In some embodiments of the compounds of Formulae (II), (IIa), (IIb), and (IIc), or the pharmaceutically acceptable salts thereof, R^{1c2} is C₁₋₆alkyl.

In some embodiments of the compounds of Formulae (II), (IIa), (IIb), and (IIc), or the pharmaceutically acceptable salts thereof, R^{1c2} is halo, —CN, —N(R^{1d})₂, or —OR^{1d}. In some embodiments, R^{1d} is H or C₁₋₆alkyl. In some embodiments, R^{1d} is —(CH₂)CN or —(CH₂)ₘNH₂. In some embodiments, R^{1d} is cycloalkyl or heterocyclyl.

In some embodiments of the compounds of Formulae (II), (IIa), (IIb), and (IIc), or the pharmaceutically acceptable salts thereof, R^{1b2} is aryl, wherein the aryl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)naryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the aryl is unsubstituted. In some embodiments, the aryl is phenyl.

In some embodiments of the compounds of Formulae(II), (IIa), (IIb), and (IIc), or the pharmaceutically acceptable salts thereof, R^{1b2} is heteroaryl or heterocyclyl, wherein the heteroaryl and heterocyclyl is unsubstituted or substituted with one or more substituents selected from the group consisting of C₁₋₆alkyl, halo, —CN, —(CH₂)ₙOH, —C(O)R^{1e}, —C(O)N(R^{1e})_{Z}, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the heteroaryl or heterocyclyl is unsubstituted. In some embodiments, R^{1b2} is heteroaryl substituted with —NHC(O)R^{1e}. In some embodiments, R^{1b2} is

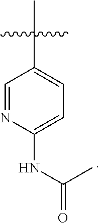 [SMILES: *c1ccc(NC(C)=O)nc1]

In some embodiments, R^{1b2} is heterocyclyl wherein the heterocyclyl is unsubstituted. In some embodiments, R^{1b2} is

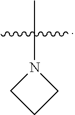 [SMILES: *N1CCC1]

In some embodiments of the compounds of Formulae (II), (IIa), (IIb), and (IIc), or the pharmaceutically acceptable salts thereof, R^{1b2} is cycloalkyl or cycloalkenyl, wherein the cycloalkyl and cycloalkenyl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the cycloalkyl or cycloalkenyl is unsubstituted.

In some embodiments of the compounds of Formulae (II), (IIa), (IIb), and (IIc), or the pharmaceutically acceptable salts thereof, R^{1b2} is C₁₋₆alkyl or alkynyl, wherein the C₁₋₆alkyl and alkynyl is unsubstituted or substituted with one or more substituents selected from the group consisting of aryl, heterocyclyl, and cycloalkyl. In some embodiments, the C₁₋₆alkyl is substituted with heterocyclyl. In some embodiments, the C₁₋₆alkyl is substituted with cycloalkyl.

In some embodiments of the compounds of Formulae (II), (IIa), (IIb), and (IIc), or the pharmaceutically acceptable salts thereof, R^{1b2} is H.

In some embodiments of the compounds of Formulae (II), (IIa), (IIb), and (IIc), or the pharmaceutically acceptable salts thereof, R^{1b2} is halo.

In some embodiments of the compounds of Formulae (II), (IIa), (IIb), and (IIc), or the pharmaceutically acceptable salts thereof, R^{1b2} is —OR^{1e} or —N(R^{1e})₂. In some embodiments, R^{1e} is C₁₋₆alkyl. In some embodiments, R^{1e} is —(CH₂)ₘCN. In some embodiments, R^{1e} is —(CH₂)ₘNH₂. In some embodiments, R^{1e} is cycloalkyl. In some embodiments, R^{1e} is heterocyclyl.

In some embodiments of the compounds of Formulae (II), (IIa), (IIb), and (IIc), or the pharmaceutically acceptable salts thereof, X²⁰ is CR^{20a}. In some embodiments, X²⁰ is CR^{20a} wherein R^{20a} is H. In some embodiments, X²⁰ is CH. In some embodiments, X²⁰ is CR^{20a} wherein R^{20a} is halo, CN, aryl, or heteroaryl.

In some embodiments of the compounds of Formulae (II), (IIa), (IIb), and (IIc), or the pharmaceutically acceptable salts thereof, X²⁰ is N.

In some embodiments of the compounds of Formulae (II), (IIa), (IIb), and (IIc), or the pharmaceutically acceptable salts thereof, X²¹ is CH.

In some embodiments of the compounds of Formulae (II), (IIa), (IIb), and (IIc), or the pharmaceutically acceptable salts thereof, X²¹ is N.

In some embodiments of the compounds of Formulae (II), (IIa), (IIb), and (IIc) or the pharmaceutically acceptable salts thereof, X²⁰ is N and X²¹ is CH.

In some embodiments of the compounds of Formulae (II), (IIa), (IIb), and (IIc), or the pharmaceutically acceptable salts thereof, R^{2b} is C₁₋₆alkyl, wherein the C₁₋₆alkyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is C₁₋₆alkyl, wherein the C₁₋₆alkyl is unsubstituted. In some embodiments, R^{2b} is —CH₃, —CH₂CH₃, or

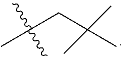 [SMILES: *CC(C)(C)C]

In some embodiments, R^{2b} is C₁₋₆alkyl, wherein the C₁₋₆alkyl is substituted with one or more substituents selected from the group consisting of cycloalkyl and halocycloalkyl. In some embodiments, R^{2b} is

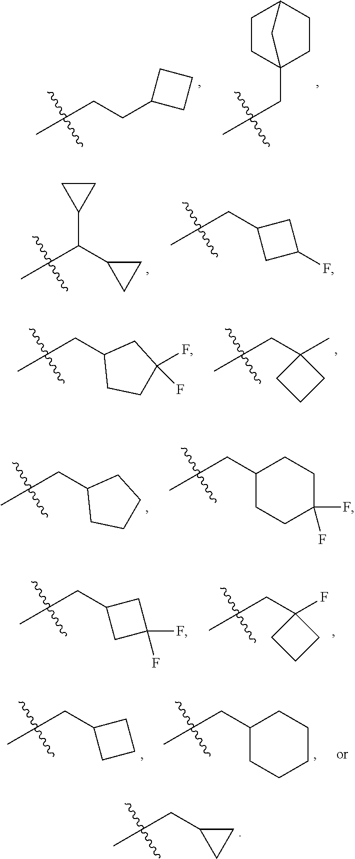

In some embodiments, R^{2b} is

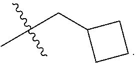 [SMILES: *CC1CCC1]

In some embodiments, R^{2b} is C₁₋₆alkyl, wherein the C₁₋₆alkyl is substituted with one or more heterocyclyl. In some embodiments, R^{2b} is

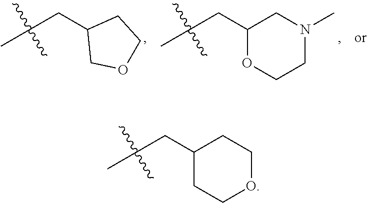

In some embodiments, R^{2b} is C₁₋₆alkyl, wherein the C₁₋₆alkyl is substituted with one or more substituents selected from the group consisting of cycloalkyl, aryl, and haloaryl. In some embodiments, R^{2b} is

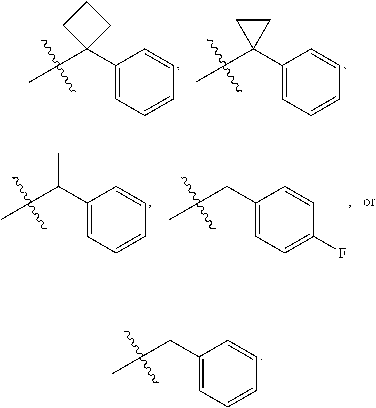

In some embodiments, R^{2b} is C₁₋₆alkyl, wherein the C₁₋₆alkyl is substituted with one or more substituents selected from the group consisting of cycloalkyl and OH. In some OH embodiments, R^{2b} is

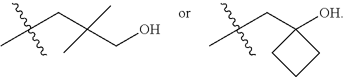

In some embodiments of the compounds of Formulae (II), (IIa), (IIb), and (IIc), or the pharmaceutically acceptable salts thereof, R^{2b} is cycloalkyl, wherein the cycloalkyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is cycloalkyl wherein the cycloalkyl is unsubstituted. In some embodiments, R^{2b} is

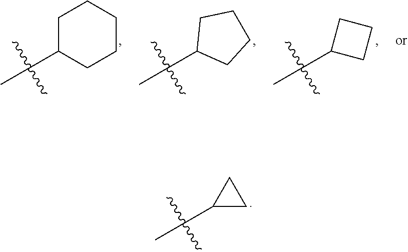

In some embodiments of the compounds of Formulae (II), (IIa), (IIb), and (IIc) or the pharmaceutically acceptable salts thereof, R^{2b} is aryl, wherein the aryl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is aryl and is

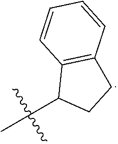 [SMILES: *C1CCc2ccccc21]

In some embodiments of the compounds of Formulae (II), (IIa), (IIb), and (IIc), or the pharmaceutically acceptable salts thereof, R^{2b} is selected from the group consisting of heteroaryl and heterocyclyl, wherein the heteroaryl and heterocyclyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is selected from the group consisting of heteroaryl and heterocyclyl wherein the heteroaryl and heterocyclyl is unsubstituted. In some embodiments, R^{2b} is selected from the group consisting of heteroaryl and heterocyclyl, wherein the heteroaryl and heterocyclyl is substituted with one or more C₁₋₆alkyl. In some embodiments, R^{2b} is

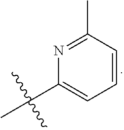 [SMILES: *c1cccc(C)n1]

In some embodiments, R^{2b} is selected from the group consisting of heteroaryl and heterocyclyl, wherein the heteroaryl and heterocyclyl is substituted with one or more alkoxy. In some embodiments, R^{2b} is

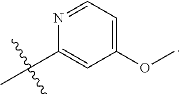 [SMILES: *c1cc(OC)ccn1]

In some embodiments, R^{2b} is selected from the group consisting of heteroaryl and heterocyclyl, wherein the heteroaryl and heterocyclyl is substituted with one or more heterocyclyl wherein the heterocyclyl is unsubstituted or substituted with C1-6alkyl. In some embodiments, R^{2b} is

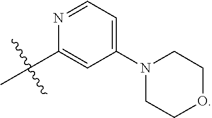 [SMILES: *c1cc(N2CCOCC2)ccn1]

In some embodiments of the compounds of Formulae (II), (IIa), (IIb), and (IIc), or the pharmaceutically acceptable salts thereof, R^{2b} is selected from the group consisting of —CH₃, —CH₂CH₃,

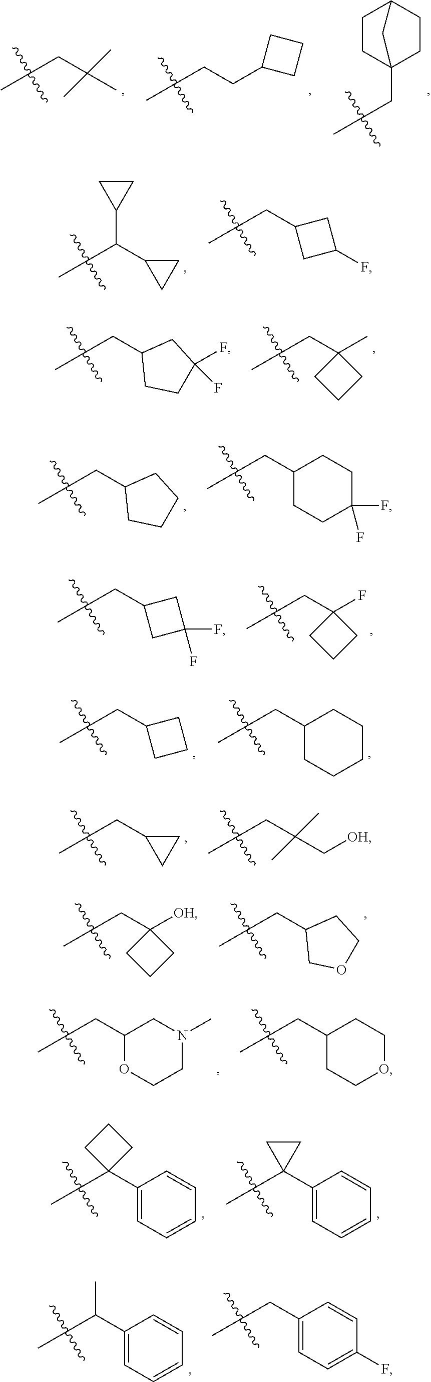

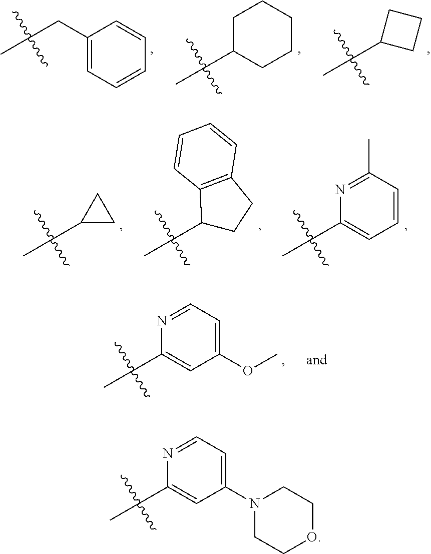

In some embodiments, R^{2b} is

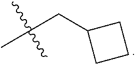 [SMILES: *CC1CCC1]

In some embodiments, the compound is of Formula (III):

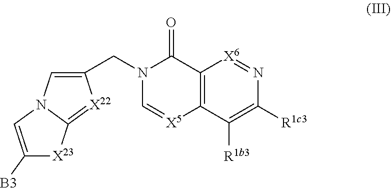

or a pharmaceutically acceptable salt thereof, wherein:
X⁵ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X⁶ is CH or N;
R^{1b3} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b3} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b3} is unsubstituted or substituted with one or more substituents selected from the group consisting of aryl, heterocyclyl, and cycloalkyl;
R^{1c3} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²² is CR^{22a} or N; wherein R^{22a} is H, halo, aryl, or heteroaryl;
X²³ is CH, S, O, or NH;
B3 is selected from the group consisting of H, C₁₋₆alkyl, CN, —CH₂—NR²R^{2b},

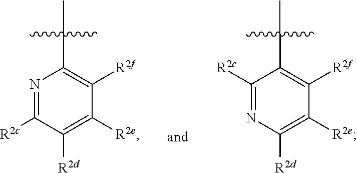

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

In some embodiments of the compounds of Formula (III) or the pharmaceutically acceptable salts thereof, X⁵ is CR^{1a}. In some embodiments, R^{1a} is H. In some embodiments, R^{1a} is C₁₋₆alkyl, wherein the C₁₋₆alkyl is unsubstituted or substituted with aryl. In some embodiments, R^{1a} is halo, cycloalkyl, or heteroaryl.

In some embodiments of the compounds of Formula (III) or the pharmaceutically acceptable salts thereof, X⁵ is N.

In some embodiments of the compounds of Formula (III) or the pharmaceutically acceptable salts thereof, X⁶ is CH. In some embodiments, X⁵ is CR^{1a} and X⁶ is CH.

In some embodiments of the compounds of Formula (III) or the pharmaceutically acceptable salts thereof, X⁶ is N.

In some embodiments, the compound of Formula (III), or the pharmaceutically acceptable salt thereof, is a compound of Formula (IIIa), or a pharmaceutically acceptable salt thereof:

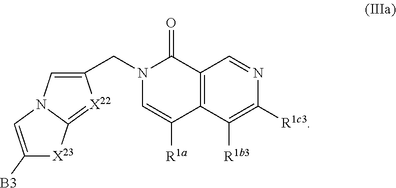

In some embodiments, the compound of Formula (III), or the pharmaceutically acceptable salt thereof, is a compound of Formula (IIIb), or a pharmaceutically acceptable salt thereof:

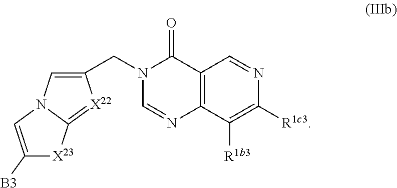

In some embodiments of the compounds of Formulae (III), (IIIa), and (IIIb) or the pharmaceutically acceptable salts thereof, B3 is —CH₂—NR^{2a}R^{2b}.

In some embodiments of the compounds of Formulae (III), (IIIa), and (IIIb) or the pharmaceutically acceptable salts thereof, R^{2a} is H. In some embodiments, R^{2a} is H and R^{2b} is selected from the group consisting of —CH₃, —CH₂CH₃,

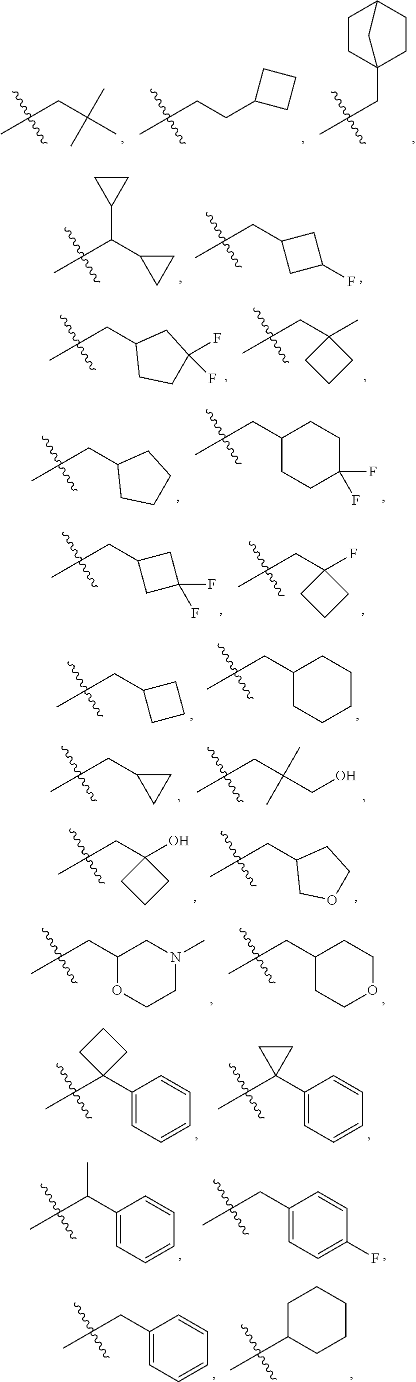

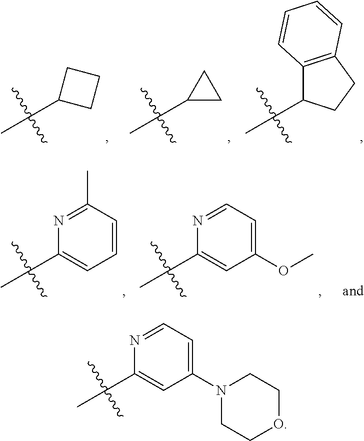

In some embodiments, R^{2a} is H and R^{2b} is

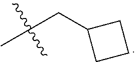 [SMILES: *CC1CCC1]

In some embodiments of the compounds of Formulae (III), (IIIa), and (IIIb) or the pharmaceutically acceptable salts thereof, R^{2a} and R^{2b} combine to form a 5-7-membered heterocyclyl, wherein the heterocyclyl is substituted with C₁₋₆alkyl. In some embodiments, R^{2a} and R^{2b} combine to form

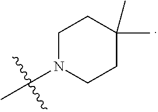 [SMILES: *N1CCC(C)(C)CC1]

In some embodiments of the compounds of Formulae (III), (IIIa), and (IIIb) or the pharmaceutically acceptable salts thereof, R^{2a} and R^{2b} combine to form a 5-7-membered heterocyclyl, wherein the heterocyclyl is unsubstituted.

In some embodiments of the compounds of Formulae (III), (IIIa), and (IIIb) or the pharmaceutically acceptable salts thereof, B3 is

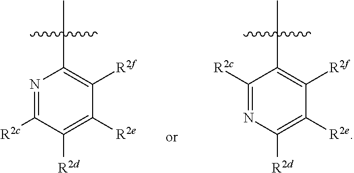

In some embodiments, one of R^{2c}, R^{2d}, R^{2e}, and R^{2f} is heterocyclyl and the others are H. In some embodiments, one of R^{2c}, R^{2d}, R^{2e}, and R^{2f} is

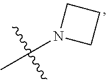 [SMILES: *N1CCC1]

and the others are H. In some embodiments, one of R^{2c}, R^{2d}, R^{2e}, and R^{2f} is —NHR^{2g} and the others are H. In some embodiments, R^{2g} is C₁₋₆alkyl, wherein the C₁₋₆alkyl is unsubstituted or substituted with cycloalkyl. In some embodiments, R^{2g} is —CH₃, —CH₂CH₃,

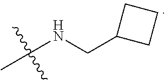 [SMILES: *NCC1CCC1]

In some embodiments R^{2g} is H. In some embodiments, one of R^{2c}, R^{2d}, R^{2e}, and R^{2f} is C₁₋₆alkyl or heterocyclyl and the others are H.

In some embodiments of the compounds of Formulae (III), (IIIa), and (IIIb) or the pharmaceutically acceptable salts thereof, B3 is C₁₋₆alkyl. In some embodiments, B3 is —CH³.

In some embodiments of the compounds of Formulae (III), (IIIa), and (IIIb) or the pharmaceutically acceptable salts thereof, B3 is H or CN.

In some embodiments, the compound of Formula (III), or the pharmaceutically acceptable salt thereof, is a compound of Formula (IIIc), or a pharmaceutically acceptable salt thereof:

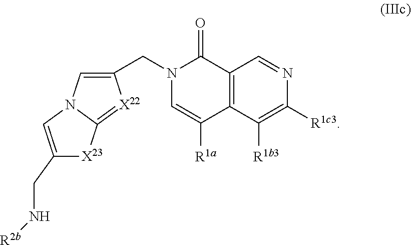

In some embodiments of the compounds of Formulae (III), (IIIa), (IIIb), and (IIIc) or the pharmaceutically acceptable salts thereof, R^{1c3} is H. In some embodiments, X⁵ is CR^{1a}, X⁶ is CH, and R^{1c3} is H.

In some embodiments of the compounds of Formula (III), (IIIa), (IIIb), and (IIIc) or the pharmaceutically acceptable salts thereof, R^{1c3} is C₁₋₆alkyl.

In some embodiments of the compounds of Formula (III), (IIIa), (IIIb), and (IIIc) or the pharmaceutically acceptable salts thereof, R^{1c3} is halo, —CN, —N(R^{1d})₂, or —OR^{1d}. In some embodiments, R^{1d} is H or C₁₋₆alkyl. In some embodiments, R^{1d} is —(CH₂)CN or —(CH₂)ₘNH₂. In some embodiments, R^{1d} is cycloalkyl or heterocyclyl.

In some embodiments of the compounds of Formula (III), (IIIa), (IIIb), and (IIIc) or the pharmaceutically acceptable salts thereof, R^{1b3} is aryl, wherein the aryl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the aryl is unsubstituted. In some embodiments, the aryl is phenyl.

In some embodiments of the compounds of Formula (III), (IIIa), (IIIb), and (IIIc) or the pharmaceutically acceptable salts thereof, R^{1b3} is heteroaryl or heterocyclyl, wherein the heteroaryl and heterocyclyl is unsubstituted or substituted with one or more substituents selected from the group consisting of C₁₋₆alkyl, halo, —CN, —(CH₂)ₙOH, —C(O)R^{1e}, —C(O)N(R^{1e}), —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the heteroaryl or heterocyclyl is unsubstituted. In some embodiments, R^{1b3} is heteroaryl substituted with —NHC(O)R^{1e}. In some embodiments, R^{1b3} is

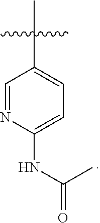 [SMILES: *c1ccc(NC(C)=O)nc1]

In some embodiments, R^{1b3} is heterocyclyl wherein the heterocyclyl is unsubstituted. In some embodiments, R^{1b3} is

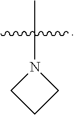 [SMILES: *N1CCC1]

In some embodiments of the compounds of Formula (III), (IIIa), (IIIb), and (IIIc) or the pharmaceutically acceptable salts thereof, R^{1b3} is cycloalkyl or cycloalkenyl, wherein the cycloalkyl and cycloalkenyl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the cycloalkyl or cycloalkenyl is unsubstituted.

In some embodiments of the compounds of Formula (III), (IIIa), (IIIb), and (IIIc) or the pharmaceutically acceptable salts thereof, R^{1b3} is C₁₋₆alkyl or alkynyl, wherein the C₁₋₆alkyl and alkynyl is unsubstituted or substituted with one or more substituents selected from the group consisting of aryl, heterocyclyl, and cycloalkyl. In some embodiments, the C₁₋₆alkyl is substituted with heterocyclyl. In some embodiments, the C₁₋₆alkyl is substituted with cycloalkyl.

In some embodiments of the compounds of Formula (III), (IIIa), (IIIb), and (IIIc) or the pharmaceutically acceptable salts thereof, R^{1b3} is H.

In some embodiments of the compounds of Formula (III), (IIIa), (IIIb), and (IIIc) or the pharmaceutically acceptable salts thereof, R^{1b3} is halo.

In some embodiments of the compounds of Formula (III), (IIIa), (IIIb), and (IIIc) or the pharmaceutically acceptable salts thereof, R^{1b3} is —OR^{1e} or —N(R^{1e})₂. In some embodiments, R^{1e} is C₁₋₆alkyl. In some embodiments, R^{1e} is —(CH₂)ₘCN. In some embodiments, R^{1e} is —(CH₂)ₘNH₂. In some embodiments, R^{1e} is cycloalkyl. In some embodiments, R^{1e} is heterocyclyl.

In some embodiments of the compounds of Formulae (III), (IIIa), (IIIb), and (IIIc) or the pharmaceutically acceptable salts thereof, X²² is CR^{22a}. In some embodiments, X²² is CR^{22a} wherein R^{22a} is H. In some embodiments, X²² is CH. In some embodiments, X²² is CR^{22a} wherein R^{22a} is halo or aryl.

In some embodiments of the compounds of Formulae (III), (IIIa), (IIIb), and (IIIc) or the pharmaceutically acceptable salts thereof, X²² is N.

In some embodiments of the compounds of Formulae (III), (IIIa), (IIIb), and (IIIc) or the pharmaceutically acceptable salts thereof, X²³ is CH₂.

In some embodiments of the compounds of Formulae (III), (IIIa), (IIIb), and (IIIc) or the pharmaceutically acceptable salts thereof, X²³ is NH.

In some embodiments of the compounds of Formulae (III), (IIIa), (IIIb), and (IIIc) or the pharmaceutically acceptable salts thereof, X²³ is S or O.

In some embodiments of the compounds of Formulae (III), (IIIa), (IIIb), and (IIIc) or the pharmaceutically acceptable salts thereof, X²² is N and X²³ is S.

In some embodiments of the compounds of Formulae (III), (IIIa), (IIIb), and (IIIc) or the pharmaceutically acceptable salts thereof, R^{2b} is C₁₋₆alkyl, wherein the C₁₋₆alkyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is C₁₋₆alkyl, wherein the C₁₋₆alkyl is unsubstituted. In some embodiments, R^{2b} is —CH₃, —CH₂CH₃, or

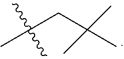 [SMILES: *CC(C)(C)C]

In some embodiments, R^{2b} is C₁₋₆alkyl, wherein the C₁₋₆alkyl is substituted with one or more substituents selected from the group consisting of cycloalkyl and halocycloalkyl. In some embodiments, R^{2b} is

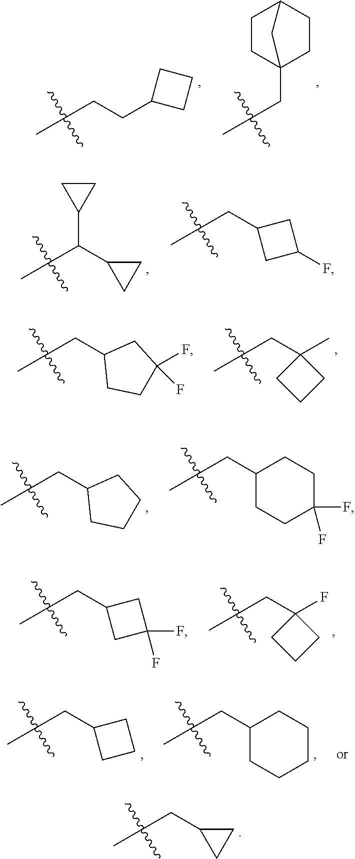

In some embodiments, R^{2b} is

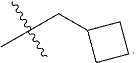 [SMILES: *CC1CCC1]

In some embodiments, R^{2b} is C₁₋₆alkyl, wherein the C₁₋₆alkyl is substituted with one or more heterocyclyl. In some embodiments, R^{2b} is

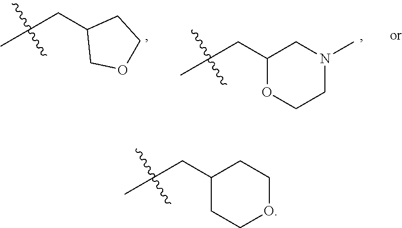

In some embodiments, R^{2b} is C₁₋₆alkyl, wherein the C₁₋₆alkyl is substituted with one or more substituents selected from the group consisting of cycloalkyl, aryl, and haloaryl. In some embodiments, R^{2b} is

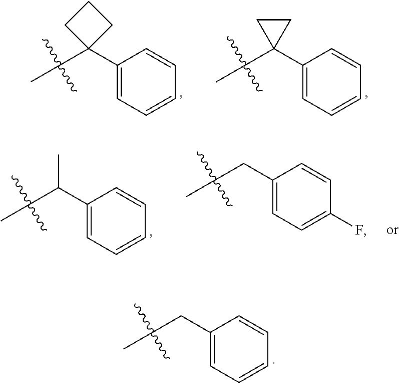

In some embodiments, R^{2b} is C₁₋₆alkyl, wherein the C₁₋₆alkyl is substituted with one or more substituents selected from the group consisting of cycloalkyl and OH. In some embodiments, R^{2b} is

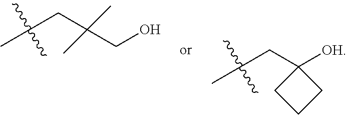

In some embodiments of the compounds of Formulae (III), (IIIa), (IIIb), and (IIIc) or the pharmaceutically acceptable salts thereof, R^{2b} is cycloalkyl, wherein the cycloalkyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is cycloalkyl wherein the cycloalkyl is unsubstituted. In some embodiments, R^{2b} is

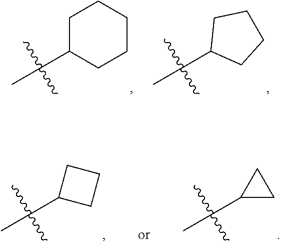

In some embodiments of the compounds of Formulae (III), (IIIa), (IIIb), and (IIIc) or the pharmaceutically acceptable salts thereof, R^{2b} is aryl, wherein the aryl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is aryl and is

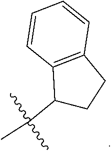 [SMILES: *C1CCc2ccccc21]

In some embodiments of the compounds of Formulae (III), (IIIa), (IIIb), and (IIIc) or the pharmaceutically acceptable salts thereof, R^{2b} is selected from the group consisting of heteroaryl and heterocyclyl, wherein the heteroaryl and heterocyclyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is selected from the group consisting of heteroaryl and heterocyclyl wherein the heteroaryl and heterocyclyl is unsubstituted. In some embodiments, R^{2b} is selected from the group consisting of heteroaryl and heterocyclyl, wherein the heteroaryl and heterocyclyl is substituted with one or more C₁₋₆alkyl. In some embodiments, R^{2b} is

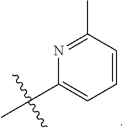 [SMILES: *c1cccc(C)n1]

In some embodiments, R^{2b} is selected from the group consisting of heteroaryl and heterocyclyl, wherein the heteroaryl and heterocyclyl is substituted with one or more alkoxy. In some embodiments, R^{2b} is

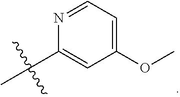 [SMILES: *c1cc(OC)ccn1]

n some embodiments, R^{2b} is selected from the group consisting of heteroaryl and heterocyclyl, wherein the heteroaryl and heterocyclyl is substituted with one or more heterocyclyl wherein the heterocyclyl is unsubstituted or substituted with C₁₋₆alkyl. In some embodiments, R^{2b} is

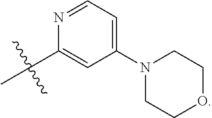 [SMILES: *c1cc(N2CCOCC2)ccn1]

In some embodiments of the compounds of Formulae (III), (IIIa), (IIIb), and (IIIc) or the pharmaceutically acceptable salts thereof, R^{2b} is selected from the group consisting of —CH₃, —CH₂CH₃,

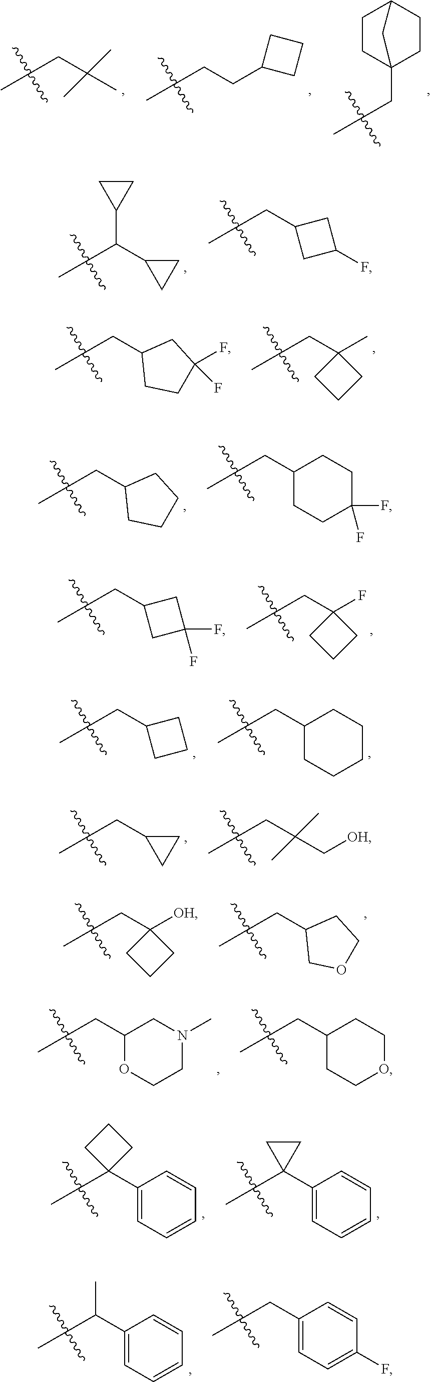

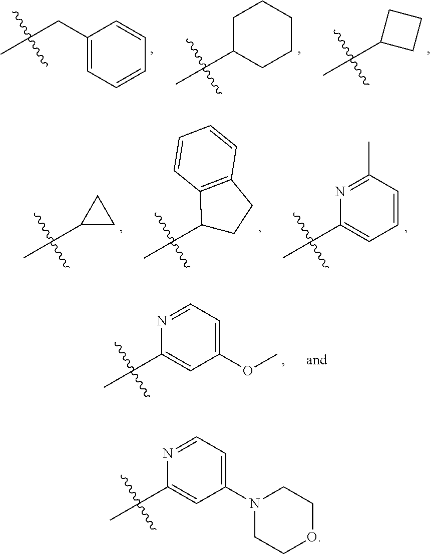

In some embodiments of R^{2b} is

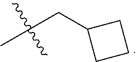 [SMILES: *CC1CCC1]

In some embodiments, the compound is of Formula (IV):

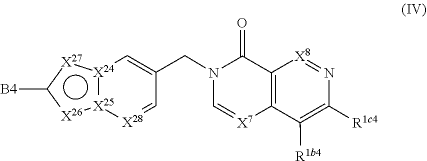

or a pharmaceutically acceptable salt thereof, wherein:
X⁷ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X⁸ is CH or N;
R^{1b4} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b4} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b4} is unsubstituted or substituted with one or more substituents selected from the group consisting of aryl, heterocyclyl, and cycloalkyl;
R^{1c4} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁴ and X²⁵ are independently C or N;
X²⁶ and X²⁷ are independently CH, O, S, N, or NR^{26a}; wherein R²⁶ is H or C₁₋₆alkyl;
X²⁸ is CH or N;
B4 is selected from the group consisting of H, C₁₋₆alkyl, CN, —CH₂—NR^{2a}R^{2b},

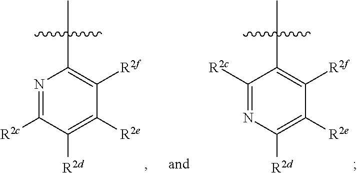

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

In some embodiments of the compounds of Formula (IV) or the pharmaceutically acceptable salts thereof, X⁷ is CR^{1a}. In some embodiments, R^{1a} is H. In some embodiments, R^{1a} is C₁₋₆alkyl, wherein the C₁₋₆alkyl is unsubstituted or substituted with aryl. In some embodiments, R^{1a} is halo, cycloalkyl, or heteroaryl.

In some embodiments of the compounds of Formula (IV) or the pharmaceutically acceptable salts thereof, X⁷ is N.

In some embodiments of the compounds of Formula (IV) or the pharmaceutically acceptable salts thereof, X⁸ is CH. In some embodiments, X⁷ is CR^{1a} and X⁸ is CH.

In some embodiments of the compounds of Formula (IV) or the pharmaceutically acceptable salts thereof, X⁸ is N.

In some embodiments, the compound of Formula (IV), or the pharmaceutically acceptable salt thereof, is a compound of Formula (IVa), or a pharmaceutically acceptable salt thereof:

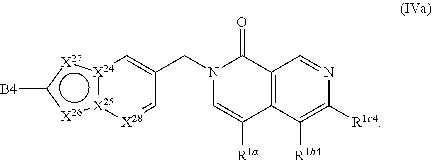

In some embodiments, the compound of Formula (IV), or the pharmaceutically acceptable salt thereof, is a compound of Formula (IVb), or a pharmaceutically acceptable salt thereof:

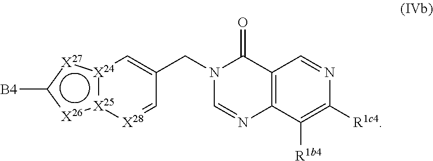

In some embodiments of the compounds of Formulae (IV), (IVa), and (IVb) or the pharmaceutically acceptable salts thereof, B4 is —CH₂—NR^{2a}R^{2b}.

In some embodiments of the compounds of Formulae (IV), (IVa), and (IVb) or the pharmaceutically acceptable salts thereof, R^{2a} is H. In some embodiments, R^{2a} is H and R^{2b} is selected from the group consisting of —CH₃, —CH₂CH₃,

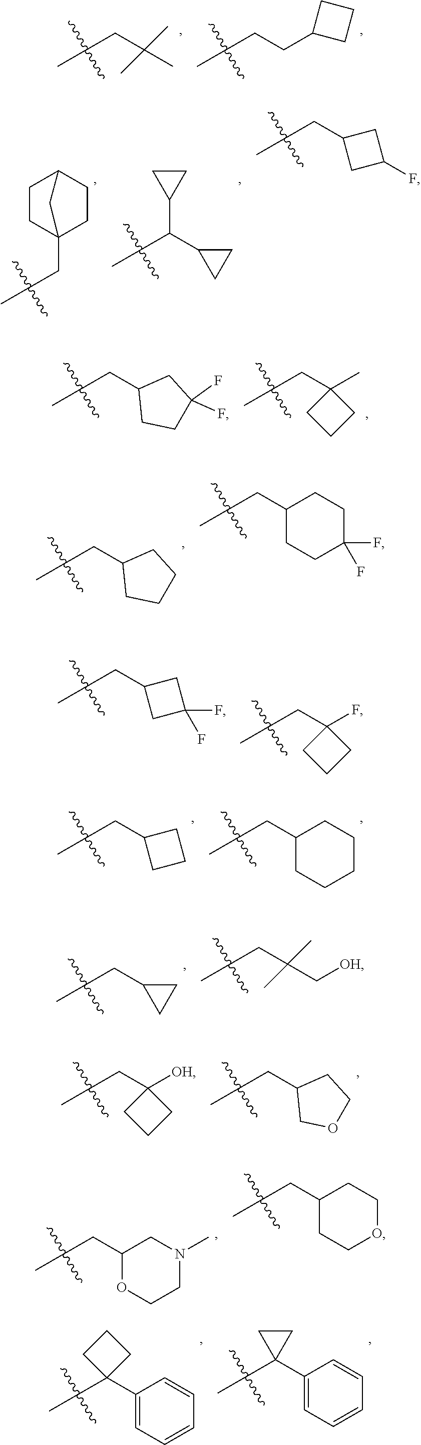

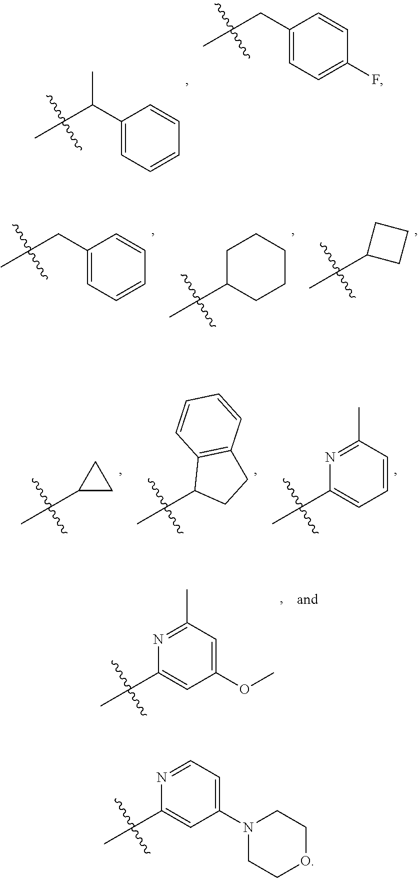

In some embodiments, R^{2a} is H and R^{2b} is

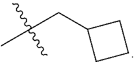 [SMILES: *CC1CCC1]

In some embodiments of the compounds of Formulae (IV), (IVa), and (IVb) or the pharmaceutically acceptable salts thereof, R^{2a} and R^{2b} combine to form a 5-7-membered heterocyclyl, wherein the heterocyclyl is substituted with C₁₋₆alkyl. In some embodiments, R² and R^{2b} combine to form

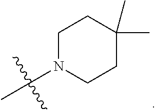 [SMILES: *N1CCC(C)(C)CC1]

In some embodiments of the compounds of Formulae (IV), (IVa), and (IVb) or the pharmaceutically acceptable salts thereof, R^{2a} and R^{2b} combine to form a 5-7-membered heterocyclyl, wherein the heterocyclyl is unsubstituted.

In some embodiments of the compounds of Formulae (IV), (IVa), and (IVb) or the pharmaceutically acceptable salts thereof, B4 is

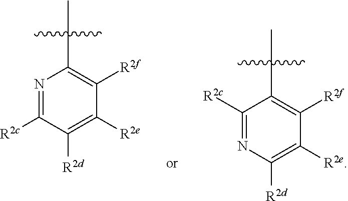

In some embodiments, one of R^{2c}, R^{2d}, R^{2e}, and R^{2f} is heterocyclyl and the others are H. In some embodiments, one of R^{2c}, R^{2d}, R^{2e}, and R^{2f} is

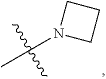 [SMILES: *N1CCC1]

and the others are H. In some embodiments, one of R^{2c}, R^{2d}, R^{2e}, and R^{2f} is —NHR^{2g} and the others are H. In some embodiments, R^{2g} is C₁₋₆alkyl, wherein the C₁₋₆alkyl is unsubstituted or substituted with cycloalkyl. In some embodiments, R^{2g} is —CH₃, —CH₂CH₃, or

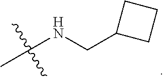 [SMILES: *NCC1CCC1]

In some embodiments R^{2g} is H. In some embodiments, one of R^{2c}, R^{2d}, R^{2e}, and R^{2f} is C₁₋₆alkyl or heterocyclyl and the others are H.

In some embodiments of the compounds of Formulae (IV), (IVa), and (IVb) or the pharmaceutically acceptable salts thereof, B4 is C₁₋₆alkyl. In some embodiments, B4 is —CH³.

In some embodiments of the compounds of Formulae (IV), (IVa), and (IVb) or the pharmaceutically acceptable salts thereof, B4 is H or CN.

In some embodiments, the compound of Formula (IV), or the pharmaceutically acceptable salt thereof, is a compound of Formula (IVc), or a pharmaceutically acceptable salt thereof:

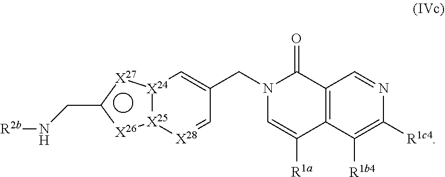

In some embodiments of the compounds of Formulae (IV), (IVa), (IVb), and (IVc) or the pharmaceutically acceptable salts thereof, R^{1c4} is H. In some embodiments, X⁷ is CR^{1a}, X⁸ is CH, and R^{1c4} is H.

In some embodiments of the compounds of Formula (IV), (IVa), (IVb), and (IVc) or the pharmaceutically acceptable salts thereof, R^{1c4} is C₁₋₆alkyl.

In some embodiments of the compounds of Formula (IV), (IVa), (IVb), and (IVc) or the pharmaceutically acceptable salts thereof, R^{1c4} is halo, —CN, —N(R^{1d})₂, or —OR^{1d}. In some embodiments, R^{1d} is H or C₁₋₆alkyl. In some embodiments, R^{1d} is —(CH₂)ₘCN or —(CH₂)ₘNH₂. In some embodiments, R^{1d} is cycloalkyl or heterocyclyl.

In some embodiments of the compounds of Formula (IV), (IVa), (IVb), and (IVc) or the pharmaceutically acceptable salts thereof, R^{1b4} is aryl, wherein the aryl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the aryl is unsubstituted. In some embodiments, the aryl is phenyl.

In some embodiments of the compounds of Formula (IV), (IVa), (IVb), and (IVc) or the pharmaceutically acceptable salts thereof, R^{1b4} is heteroaryl or heterocyclyl, wherein the heteroaryl and heterocyclyl is unsubstituted or substituted with one or more substituents selected from the group consisting of C₁₋₆alkyl, halo, —CN, —(CH₂)ₙOH, —C(O)R^{1e}, —C(O)N(R^{1e}), —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the heteroaryl or heterocyclyl is unsubstituted. In some embodiments, R^{1b4} is heteroaryl substituted with —NHC(O)R^{1e}. In some embodiments, R^{1b4} is

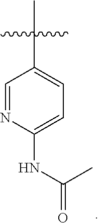 [SMILES: *c1ccc(NC(C)=O)nc1]

In some embodiments, R^{1b4} is heterocyclyl wherein the heterocyclyl is unsubstituted. In some embodiments, R^{1b4} is

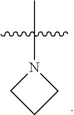 [SMILES: *N1CCC1]

In some embodiments of the compounds of Formula (IV), (IVa), (IVb), and (IVc) or the pharmaceutically acceptable salts thereof, R^{1b4} is cycloalkyl or cycloalkenyl, wherein the cycloalkyl and cycloalkenyl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the cycloalkyl or cycloalkenyl is unsubstituted.

In some embodiments of the compounds of Formula (IV), (IVa), (IVb), and (IVc) or the pharmaceutically acceptable salts thereof, R^{1b4} is C₁₋₆alkyl or alkynyl, wherein the C₁₋₆alkyl and alkynyl is unsubstituted or substituted with one or more substituents selected from the group consisting of aryl, heterocyclyl, and cycloalkyl. In some embodiments, the C₁₋₆alkyl is substituted with heterocyclyl. In some embodiments, the C₁₋₆alkyl is substituted with cycloalkyl.

In some embodiments of the compounds of Formula (IV), (IVa), (IVb), and (IVc) or the pharmaceutically acceptable salts thereof, R^{1b4} is H.

In some embodiments of the compounds of Formula (IV), (IVa), (IVb), and (IVc) or the pharmaceutically acceptable salts thereof, R^{1b4} is halo.

In some embodiments of the compounds of Formula (IV), (IVa), (IVb), and (IVc) or the pharmaceutically acceptable salts thereof, R^{1b4} is —OR^{1e} or —N(R^{1e})₂. In some embodiments, R^{1e} is C₁₋₆alkyl. In some embodiments, R^{1e} is —(CH₂)ₘCN. In some embodiments, R^{1e} is —(CH₂)ₘNH₂. In some embodiments, R^{1e} is cycloalkyl. In some embodiments, R^{1e} is heterocyclyl.

In some embodiments of the compounds of Formulae (IV), (IVa), (IVb), and (IVc), or the pharmaceutically acceptable salts thereof, X²⁴ and X²⁵ are C.

In some embodiments of the compounds of Formulae (IV), (IVa), (IVb), and (IVc), or the pharmaceutically acceptable salts thereof, X²⁴ is N and X²⁵ is C.

In some embodiments of the compounds of Formulae (IV), (IVa), (IVb), and (IVc), or the pharmaceutically acceptable salts thereof, X²⁶ and X²⁷ are independently N or NR²⁶ wherein R^{26a} s H or C₁₋₆alkyl. In some embodiments, X²⁶ and X²⁷ are independently N or NCH₃. In some embodiments, X²⁶ and X²⁷ are independently N or NH.

In some embodiments of the compounds of Formulae (IV), (IVa), (IVb), and (IVc), or the pharmaceutically acceptable salts thereof, X²⁶ and X²⁷ are CH.

In some embodiments of the compounds of Formulae (IV), (IVa), (IVb), and (IVc), or the pharmaceutically acceptable salts thereof, X²⁸ is CH.

In some embodiments of the compounds of Formulae (IV), (IVa), (IVb), and (IVc), or the pharmaceutically acceptable salts thereof, X²⁸ is N.

In some embodiments of the compounds of Formulae (IV), (IVa), (IVb), and (IVc), or the pharmaceutically acceptable salts thereof, X²⁴ and X²⁵ are C and X²⁸ is CH.

In some embodiments of the compounds of Formulae (IV), (IVa), (IVb), and (IVc), or the pharmaceutically acceptable salts thereof, X²⁶ and X²⁷ are independently N or NR²⁶ wherein R^{26a} (is H or C₁₋₆alkyl, and X²⁸ is CH.

In some embodiments of the compounds of Formulae (IV), (IVa), (IVb), and (IVc), or the pharmaceutically acceptable salts thereof, X²⁴ and X²⁵ are C, X²⁶ and X²⁷ are independently N or NR^{26a} wherein R²⁶ is H or C₁₋₆alkyl, and X²⁸ is CH.

In some embodiments of the compounds of Formulae (IV), (IVa), (IVb), and (IVc), or the pharmaceutically acceptable salts thereof, R^{2b} is C₁₋₆alkyl, wherein the C₁₋₆alkyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is C₁₋₆alkyl, wherein the C₁₋₆alkyl is unsubstituted. In some embodiments, R^{2b} is —CH₃, —CH₂CH₃, or

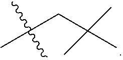 [SMILES: *CC(C)(C)C]

In some embodiments, R^{2b} is C₁₋₆alkyl, wherein the C₁₋₆alkyl is substituted with one or more substituents selected from the group consisting of cycloalkyl and halocycloalkyl. In some embodiments R^{2b} is

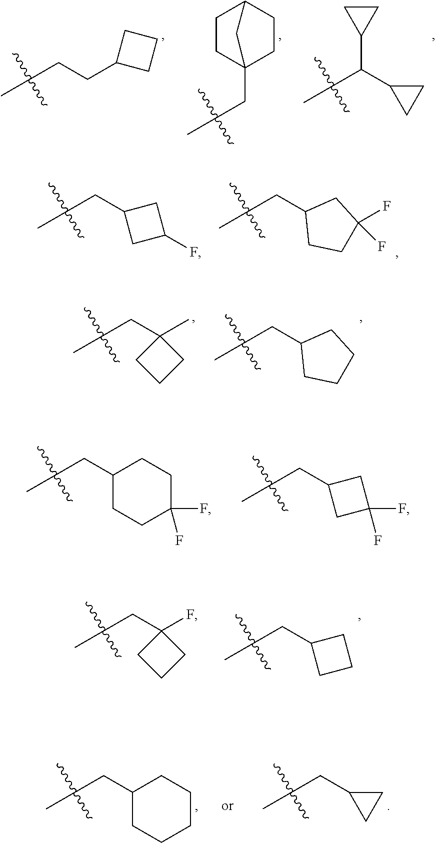

In some embodiments, R^{2b} is

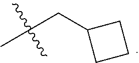 [SMILES: *CC1CCC1]

n some embodiments, R^{2b} is C₁₋₆alkyl, wherein the C₁₋₆alkyl is substituted with one or more heterocyclyl. In some embodiments, R^{2b} is

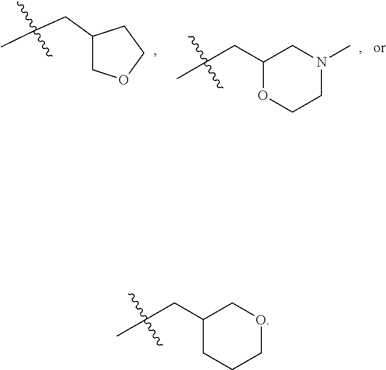

In some embodiments, R^{2b} is C₁₋₆alkyl, wherein the C₁₋₆alkyl is substituted with one or more substituents selected from the group consisting of cycloalkyl, aryl, and haloaryl. In some embodiments, R^{2b} is

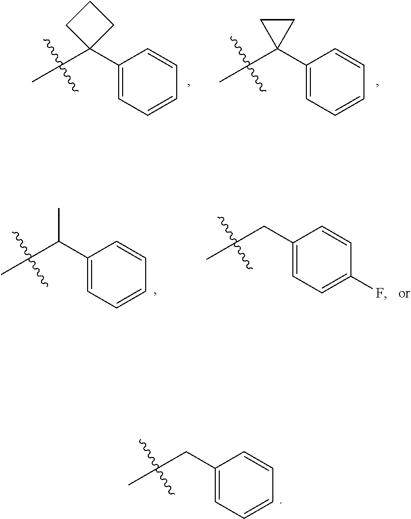

In some embodiments, R^{2b} is C₁₋₆alkyl, wherein the C₁₋₆alkyl is substituted with one or more substituents selected from the group consisting of cycloalkyl and OH. In some embodiments, R^{2b} is

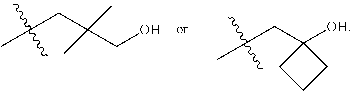

In some embodiments of the compounds of Formulae (IV), (IVa), (IVb), and (IVc), or the pharmaceutically acceptable salts thereof, R^{2b} is cycloalkyl, wherein the cycloalkyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is cycloalkyl wherein the cycloalkyl is unsubstituted. In some embodiments, R^{2b} is

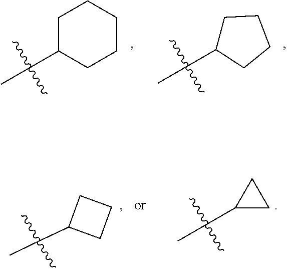

In some embodiments of the compounds of Formulae (IV), (IVa), (IVb), and (IVc), or the pharmaceutically acceptable salts thereof, R^{2b} is aryl, wherein the aryl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is aryl and is

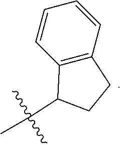 [SMILES: *C1CCc2ccccc21]

In some embodiments of the compounds of Formulae (IV), (IVa), (IVb), and (IVc), or the pharmaceutically acceptable salts thereof, R^{2b} is selected from the group consisting of heteroaryl and heterocyclyl, wherein the heteroaryl and heterocyclyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl. In some embodiments, R^{2b} is selected from the group consisting of heteroaryl and heterocyclyl wherein the heteroaryl and heterocyclyl is unsubstituted. In some embodiments, R^{2b} is selected from the group consisting of heteroaryl and heterocyclyl, wherein the heteroaryl and heterocyclyl is substituted with one or more C₁₋₆alkyl.

In some embodiments, R^{2b} is

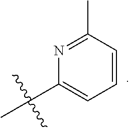 [SMILES: *c1cccc(C)n1]

In some embodiments, R^{2b} is selected from the group consisting of heteroaryl and heterocyclyl, wherein the heteroaryl and heterocyclyl is substituted with one or more alkoxy. In some embodiments, R^{2b} is

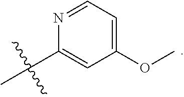 [SMILES: *c1cc(OC)ccn1]

In some embodiments, R^{2b} is selected from the group consisting of heteroaryl and heterocyclyl, wherein the heteroaryl and heterocyclyl is substituted with one or more heterocyclyl wherein the heterocyclyl is unsubstituted or substituted with C₁₋₆alkyl. In some embodiments, R^{2b} is

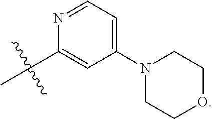 [SMILES: *c1cc(N2CCOCC2)ccn1]

In some embodiments of the compounds of Formulae (IV), (IVa), (IVb), and (IVc), or the pharmaceutically acceptable salts thereof, R^{2b} is selected from the group consisting of —CH₃, —CH₂CH₃,

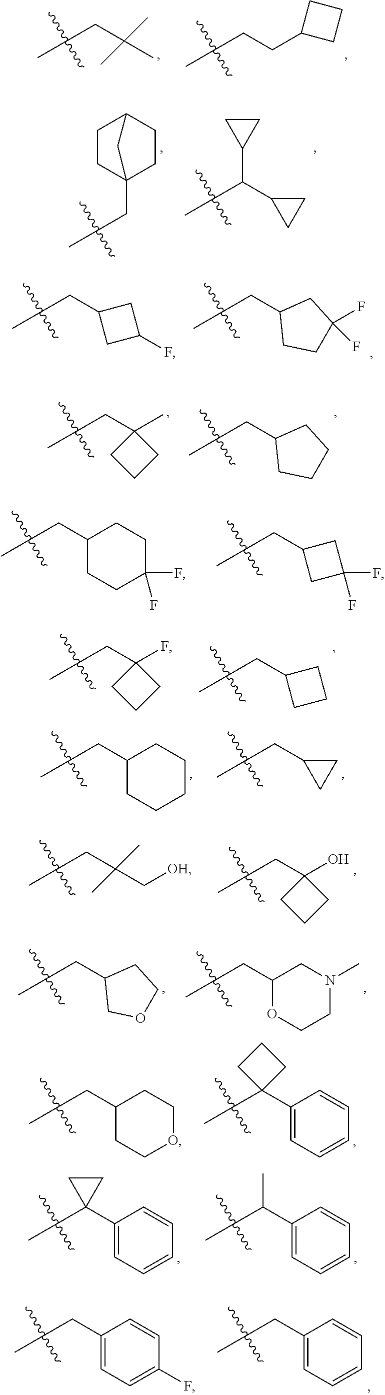

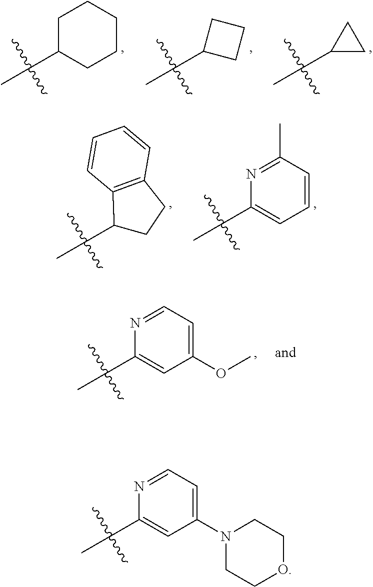

In some embodiments, R^{2b} is

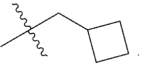 [SMILES: *CC1CCC1]

In some embodiments, the compound is of Formula (V):

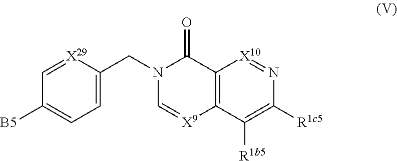

or a pharmaceutically acceptable salt thereof, wherein:
X⁹ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X¹⁰ is CH or N;
R^{1b5} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b5} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b5} is unsubstituted or substituted with one or more substituents selected from the group consisting of aryl, heterocyclyl, and cycloalkyl;
R^{1c5} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁹ is CH or N;
B5 is

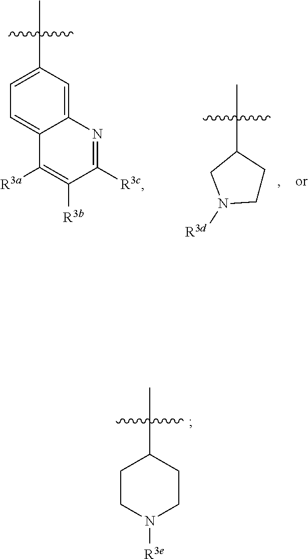

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

In some embodiments of the compounds of Formula (V) or the pharmaceutically acceptable salts thereof, X⁹ is CR^{1a}. In some embodiments, R^{1a} is H. In some embodiments, R^{1a} is C₁₋₆alkyl, wherein the C₁₋₆alkyl is unsubstituted or substituted with aryl. In some embodiments, R^{1a} is halo, cycloalkyl, or heteroaryl.

In some embodiments of the compounds of Formula (V) or the pharmaceutically acceptable salts thereof, X⁹ is N.

In some embodiments of the compounds of Formula (V) or the pharmaceutically acceptable salts thereof, X¹⁰ is CH. In some embodiments, X⁹ is CR^{1a} and X¹⁰ is CH.

In some embodiments of the compounds of Formula (V) or the pharmaceutically acceptable salts thereof, X¹⁰ is N.

In some embodiments, the compound of Formula (V), or the pharmaceutically acceptable salt thereof, is a compound of Formula (Va), or a pharmaceutically acceptable salt thereof:

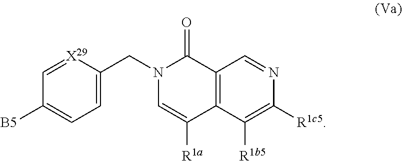

In some embodiments, the compound of Formula (V), or the pharmaceutically acceptable salt thereof, is a compound of Formula (Vb), or a pharmaceutically acceptable salt thereof:

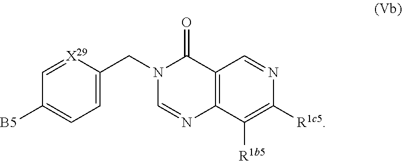

In some embodiments of the compounds of Formula (V), (Va), and (Vb) or the pharmaceutically acceptable salts thereof, R^{1c5} is H. In some embodiments, X⁹ is CR^{1a}, X¹⁰ is CH, and R^{1c5} is H.

In some embodiments of the compounds of Formula (V), (Va), and (Vb) or the pharmaceutically acceptable salts thereof, R^{1c5} is C₁₋₆alkyl.

In some embodiments of the compounds of Formula (V), (Va), and (Vb) or the pharmaceutically acceptable salts thereof, R^{1c5} is halo, —CN, —N(R^{1d})₂, or —OR^{1d}. In some embodiments, R^{1d} is H or C₁₋₆alkyl. In some embodiments, R^{1d} is —(CH₂)CN or —(CH₂)ₘNH₂. In some embodiments, R^{1d} is cycloalkyl or heterocyclyl.

In some embodiments of the compounds of Formula (V), (Va), and (Vb) or the pharmaceutically acceptable salts thereof, R^{1b5} is aryl, wherein the aryl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the aryl is unsubstituted. In some embodiments, the aryl is phenyl.

In some embodiments of the compounds of Formula (V), (Va), and (Vb) or the pharmaceutically acceptable salts thereof, R^{1b5} is heteroaryl or heterocyclyl, wherein the heteroaryl and heterocyclyl is unsubstituted or substituted with one or more substituents selected from the group consisting of C₁₋₆alkyl, halo, —CN, —(CH₂)ₙOH, —C(O)R^{1e}, —C(O)N(R^{1e}), —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the heteroaryl or heterocyclyl is unsubstituted. In some embodiments, R^{1b5} is heteroaryl substituted with —NHC(O)R^{1e}. In some embodiments, R^{1b5} is

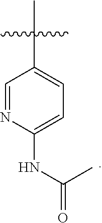 [SMILES: *c1ccc(NC(C)=O)nc1]

In some embodiments, R^{1b5} is heterocyclyl wherein the heterocyclyl is unsubstituted. In some embodiments, R^{1b5} is

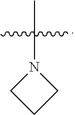 [SMILES: *N1CCC1]

In some embodiments of the compounds of Formula (V), (Va), and (Vb) or the pharmaceutically acceptable salts thereof, R^{1b5} is cycloalkyl or cycloalkenyl, wherein the cycloalkyl and cycloalkenyl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the cycloalkyl or cycloalkenyl is unsubstituted.

In some embodiments of the compounds of Formula (V), (Va), and (Vb) or the pharmaceutically acceptable salts thereof, R^{1b5} is C₁₋₆alkyl or alkynyl, wherein the C₁₋₆alkyl and alkynyl is unsubstituted or substituted with one or more substituents selected from the group consisting of aryl, heterocyclyl, and cycloalkyl. In some embodiments, the C₁₋₆alkyl is substituted with heterocyclyl. In some embodiments, the C₁₋₆alkyl is substituted with cycloalkyl.

In some embodiments of the compounds of Formula (V), (Va), and (Vb) or the pharmaceutically acceptable salts thereof, R^{1b5} is H.

In some embodiments of the compounds of Formula (V), (Va), and (Vb) or the pharmaceutically acceptable salts thereof, R^{1b5} is halo.

In some embodiments of the compounds of Formula (V), (Va), and (Vb) or the pharmaceutically acceptable salts thereof, R^{1b5} is —OR^{1e} or —N(R^{1e})₂. In some embodiments, R^{1e} is C₁₋₆alkyl. In some embodiments, R^{1e} is —(CH₂)ₘCN. In some embodiments, R^{1e} is —(CH₂)ₘNH₂. In some embodiments, R^{1e} is cycloalkyl. In some embodiments, R^{1e} is heterocyclyl.

In some embodiments of the compounds of Formulae (V), (Va), and (Vb), or the pharmaceutically acceptable salts thereof, X²⁹ is CH.

In some embodiments of the compounds of Formulae (V), (Va), and (Vb), or the pharmaceutically acceptable salts thereof, X²⁹ is N.

In some embodiments of the compounds of Formulae (V), (Va), and (Vb), or the pharmaceutically acceptable salts thereof, B5 is

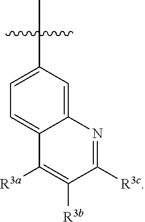

In some embodiments, one or two of R^{3a}, R^{3b}, and R^{3c} is —OR^{3x}. In some embodiments, R^{3x} is C₁₋₆alkyl. In some embodiments, one or two of R^{3a}, R^{3b}, and R^{3c} is —OCH₃. In some embodiments, one of R^{3a}, R^{3b}, and R^{3c} is —OR^{3x} and the others of R^{3a}, R^{3b}, and R^{k} are H.

In some embodiments, one or two of R^{3a}, R^{3b}, and R^{3c} is —NR^{3y}R^{3z}. In some embodiments, one or both of R^{3y} and R^{3z} is H. In some embodiments, one or both of R^{3y} and R^{3z} is C₁₋₆alkyl, wherein C₁₋₆alkyl is unsubstituted or substituted with one or more substituents selected from the group consisting of cycloalkyl and —OCH₃. In some embodiments, one or both of R^{3y} and R^{3z} is —CH₃, —CH₂CH₃,

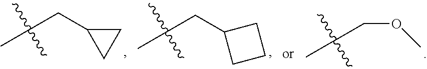

In some embodiments, one of R^{3y} and R^{3z} is cycloalkyl. In some embodiments, one of R^{3y} and R^{3z} is

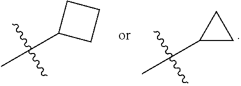

In some embodiments, both R^{3y} and R^{3z} are C₁₋₆alkyl, wherein C₁₋₆alkyl is unsubstituted. In some embodiments, one or two of R^{3a}, R^{3b}, and R^{k} is —N(CH₃)₂. In some embodiments, one of R^{3a}, R^{3b}, and R^{3c} is —NR^{3y}R^{3z} and the others of R^{3a}, R^{3b}, and R^{3c} are H.

In some embodiments, one or two of R^{3a}, R^{3b}, and R^{3c} is heterocyclyl wherein the heterocyclyl is unsubstituted or substituted with one or more substituents selected from the group consisting of C₁₋₆alkyl, halogen, and —OH. In some embodiments, one or two of R^{3a}, R^{3b}, and R^{3c} is

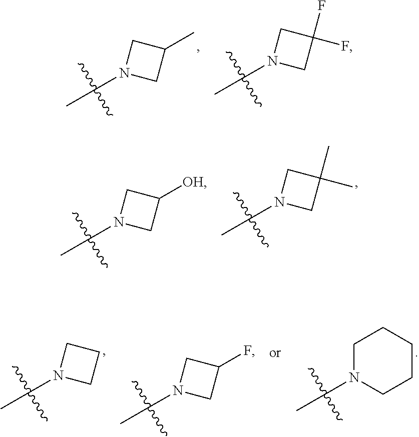

In some embodiments, one of R^{3a}, R^{3b}, and R^{3c} is heterocyclyl wherein the heterocyclyl is unsubstituted or substituted with one or more substituents selected from the group consisting of C₁₋₆alkyl, halogen, and —OH, and the others of R^{3a}, R^{3b}, and R^{3c} are H.

In some embodiments, two of R^{3a}, R^{3b}, and R^{3c} are each independently C₁₋₆alkyl, —OR^{3x}, —NR^{3y}R^{3z}, cycloalkyl, or heterocyclyl, and the other of R^{3a}, R^{3b}, and R^{3c} is H.

In some embodiments of the compounds of Formulae (V), (Va), and (Vb), or the pharmaceutically acceptable salts thereof, B5 is

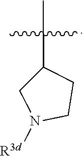

In some embodiments, R^{3d} is C₁₋₆alkyl, wherein C₁₋₆alkyl is unsubstituted or substituted with cycloalkyl.

In some embodiments of the compounds of Formulae (V), (Va), and (Vb), or the pharmaceutically acceptable salts thereof, B5 is

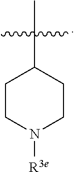

In some embodiments, R^{3e} is C₁₋₆alkyl, wherein C₁₋₆alkyl is unsubstituted or substituted with cycloalkyl.

In some embodiments, the compound is of Formula (VI):

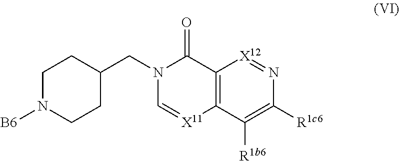

or a pharmaceutically acceptable salt thereof, wherein:
X¹¹ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X¹² is CH or N;
R^{1b6} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b6} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b6} is unsubstituted or substituted with one or more substituents selected from the group consisting of aryl, heterocyclyl, and cycloalkyl;
R^{1c6} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
B6 is

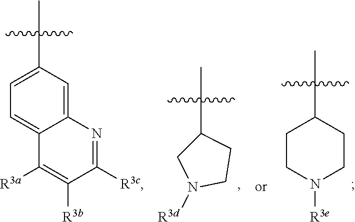

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

In some embodiments of the compounds of Formula (VI) or the pharmaceutically acceptable salts thereof, X¹¹ is CR^{1a}. In some embodiments, R^{1a} is H. In some embodiments, R^{1a} is C₁₋₆alkyl, wherein the C₁₋₆alkyl is unsubstituted or substituted with aryl. In some embodiments, R^{1a} is halo, cycloalkyl, or heteroaryl.

In some embodiments of the compounds of Formula (VI) or the pharmaceutically acceptable salts thereof, X¹¹ is N.

In some embodiments of the compounds of Formula (VI) or the pharmaceutically acceptable salts thereof, X¹² is CH. In some embodiments, X¹¹ is CR^{1a} and X¹² is CH.

In some embodiments of the compounds of Formula (VI) or the pharmaceutically acceptable salts thereof, X¹² is N.

In some embodiments, the compound of Formula (VI), or the pharmaceutically acceptable salt thereof, is a compound of Formula (VIa), or a pharmaceutically acceptable salt thereof:

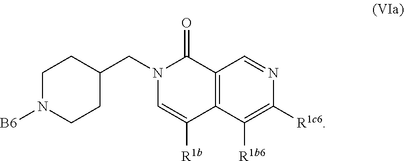

In some embodiments, the compound of Formula (VI), or the pharmaceutically acceptable salt thereof, is a compound of Formula (VIb), or a pharmaceutically acceptable salt thereof:

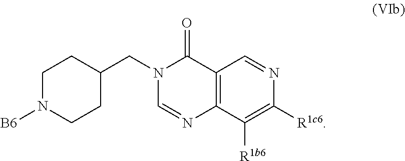

In some embodiments of the compounds of Formula (VI), (VIa), and (VIb) or the pharmaceutically acceptable salts thereof, R^{1c6} is H. In some embodiments, X¹¹ is CR^{1a}, X¹² is CH, and R^{1c6} is H.

In some embodiments of the compounds of Formula (VI), (VIa), and (VIb) or the pharmaceutically acceptable salts thereof, R^{1c6} is C₁₋₆alkyl.

In some embodiments of the compounds of Formula (VI), (VIa), and (VIb) or the pharmaceutically acceptable salts thereof, R^{1c6} is halo, —CN, —N(R^{1d})₂, or —OR^{1d}. In some embodiments, R^{1d} is H or C₁₋₆alkyl. In some embodiments, R^{1d} is —(CH₂)CN or —(CH₂)ₘNH₂. In some embodiments, R^{1d} is cycloalkyl or heterocyclyl.

In some embodiments of the compounds of Formula (VI), (VIa), and (VIb) or the pharmaceutically acceptable salts thereof, R^{1c6} is aryl, wherein the aryl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the aryl is unsubstituted. In some embodiments, the aryl is phenyl.

In some embodiments of the compounds of Formula (VI), (VIa), and (VIb) or the pharmaceutically acceptable salts thereof, R^{1c6} is heteroaryl or heterocyclyl, wherein the heteroaryl and heterocyclyl is unsubstituted or substituted with one or more substituents selected from the group consisting of C₁₋₆alkyl, halo, —CN, —(CH₂)ₙOH, —C(O)R^{1e}, —C(O)N(R^{1e}), —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the heteroaryl or heterocyclyl is unsubstituted. In some embodiments, R^{1b6} is heteroaryl substituted with —NHC(O)R^{1e}. In some embodiments, R^{1b6} is

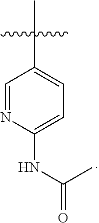 [SMILES: *c1ccc(NC(C)=O)nc1]

In some embodiments, R^{1b6} is heterocyclyl wherein the heterocyclyl is unsubstituted. In some embodiments, R^{1b6} is

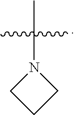 [SMILES: *N1CCC1]

In some embodiments of the compounds of Formula (VI), (VIa), and (VIb) or the pharmaceutically acceptable salts thereof, R^{1b6} is cycloalkyl or cycloalkenyl, wherein the cycloalkyl and cycloalkenyl is unsubstituted or substituted with one or more substituents selected from the group consisting of C₁₋₆alkyl, halo, —CN, —(CH₂)ₙOH, —C(O)R^{1e}, —C(O)N(R^{1e})_{Z}, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}. In some embodiments, the cycloalkyl or cycloalkenyl is unsubstituted.

In some embodiments of the compounds of Formula (VI), (VIa), and (VIb) or the pharmaceutically acceptable salts thereof, R^{1b6} is C₁₋₆alkyl or alkynyl, wherein the C₁₋₆alkyl and alkynyl is unsubstituted or substituted with one or more substituents selected from the group consisting of aryl, heterocyclyl, and cycloalkyl. In some embodiments, the C₁₋₆alkyl is substituted with aryl. In some embodiments, the C₁₋₆alkyl is substituted with heterocyclyl. In some embodiments, the C₁₋₆alkyl is substituted with cycloalkyl.

In some embodiments of the compounds of Formula (VI), (VIa), and (VIb) or the pharmaceutically acceptable salts thereof, R^{1b6} is H.

In some embodiments of the compounds of Formula (VI), (VIa), and (VIb) or the pharmaceutically acceptable salts thereof, R^{1b6} is halo.

In some embodiments of the compounds of Formula (VI), (VIa), and (VIb) or the pharmaceutically acceptable salts thereof, R^{1b6} is —OR^{1e} or —N(R^{1e})₂. In some embodiments, R^{1e} is C₁₋₆alkyl. In some embodiments, R^{1e} is —(CH₂)ₘCN. In some embodiments, R^{1e} is —(CH₂)ₘNH₂. In some embodiments, R^{1e} is cycloalkyl. In some embodiments, R^{1e} is heterocyclyl.

In some embodiments of the compounds of Formulae (VI), (VIa), and (VIb), or the pharmaceutically acceptable salts thereof, B6 is

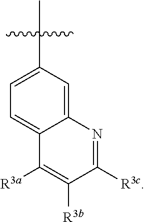

In some embodiments, one or two of R^{3a}, R^{3b}, and R^{3c} is —OR^{3x}. In some embodiments, R^{3x} is C₁₋₆alkyl. In some embodiments, one or two of R^{3a}, R^{3b}, and R^{k} is —OCH₃. In some embodiments, one of R^{3a}, R^{3b}, and R^{3c} is —OR^{3x} and the others of R^{3a}, R^{3b}, and R^{3c} are H.

In some embodiments, one or two of R^{3a}, R^{3b}, and R^{3c} is —NR^{3y}R^{3z}. In some embodiments, one or both of R^{3y} and R^{3z} is H. In some embodiments, one or both of R^{3y} and R^{3z} is C₁₋₆alkyl, wherein C₁₋₆alkyl is unsubstituted or substituted with one or more substituents selected from the group consisting of cycloalkyl and —OCH₃. In some embodiments, one or both of R^{3y} and R^{3z} is —CH₃, —CH₂CH₃, or

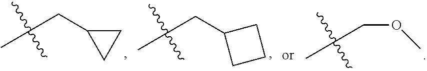

In some embodiments, one of R^{3y} and R^{3z} is cycloalkyl. In some embodiments, one of R^{3y} and R^{3z} is

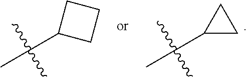

In some embodiments, both R^{3y} and R^{3z} are C₁₋₆alkyl, wherein C₁₋₆alkyl is unsubstituted. In some embodiments, one or two of R^{3a}, R^{3b}, and R^{3c} is —N(CH₃)₂. In some embodiments, one of R^{3a}, R^{3b}, and R^{3c} is —NR^{3y}R^{3z} and the others of R^{3a}, R^{3b}, and R^{3c} are H.

In some embodiments, one or two of R^{3a}, R^{3b}, and R^{3c} is heterocyclyl wherein the heterocyclyl is unsubstituted or substituted with one or more substituents selected from the group consisting of C₁₋₆alkyl, halogen, and —OH. In some embodiments, one or two of R^{3a}, R^{3b}, and R^{3c} is

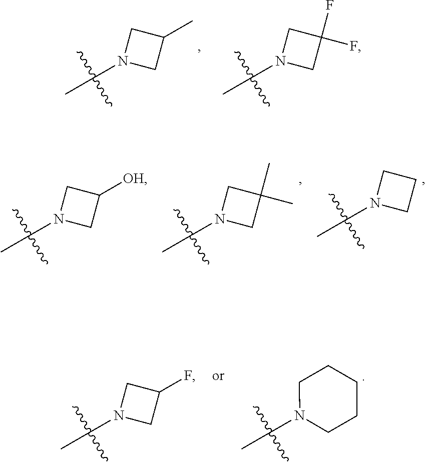

In some embodiments, one of R^{3a}, R^{3b}, and R^{3c} is heterocyclyl wherein the heterocyclyl is unsubstituted or substituted with one or more substituents selected from the group consisting of C₁₋₆alkyl, halogen, and —OH, and the others of R^{3a}, R^{3b}, and R^{3c} are H.

In some embodiments, two of R^{3a}, R^{3b}, and R^{3c} are each independently C₁₋₆alkyl, —OR^{3x}, —NR^{3y}R^{3z}, cycloalkyl, or heterocyclyl, and the other of R^{3a}, R^{3b}, and R^{3c} is H.

In some embodiments of the compounds of Formulae (VI), (VIa), (VIb), and (VIc) or the pharmaceutically acceptable salts thereof, B6 is

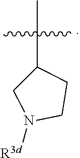

In some embodiments, R^{3d} is C₁₋₆alkyl, wherein C₁₋₆alkyl is unsubstituted or substituted with cycloalkyl.

In some embodiments of the compounds of Formulae (VI), (VIa), and (VIb), or the pharmaceutically acceptable salts thereof, B6 is

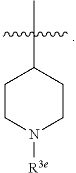

In some embodiments, R^{3e} is C₁₋₆alkyl, wherein C₁₋₆alkyl is unsubstituted or substituted with cycloalkyl.

In some embodiments, the compound, or the pharmaceutically acceptable salt thereof, is selected from the group consisting of

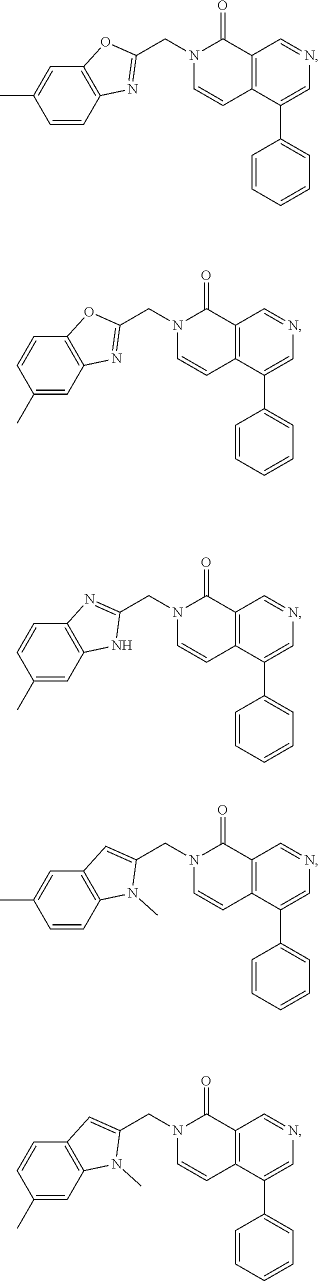 [SMILES: Cc1ccc2nc(Cn3ccc4c(-c5ccccc5)cncc4c3=O)oc2c1 ; Cc1ccc2oc(Cn3ccc4c(-c5ccccc5)cncc4c3=O)nc2c1 ; Cc1ccc2nc(Cn3ccc4c(-c5ccccc5)cncc4c3=O)[nH]c2c1 ; Cc1ccc2c(c1)cc(Cn1ccc3c(-c4ccccc4)cncc3c1=O)n2C ; Cc1ccc2cc(Cn3ccc4c(-c5ccccc5)cncc4c3=O)n(C)c2c1]

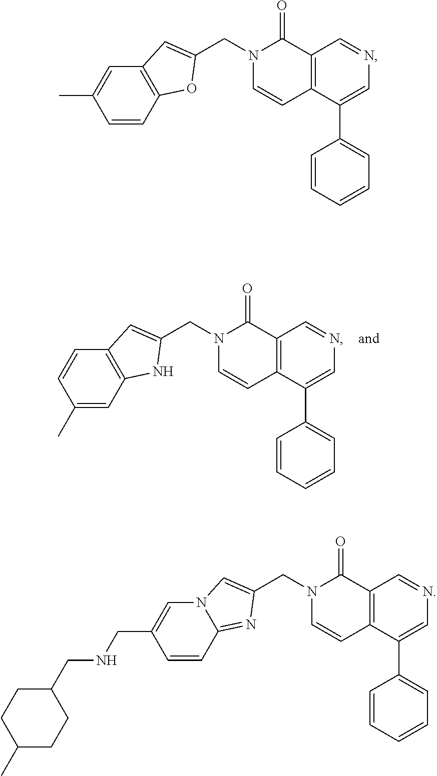 [SMILES: Cc1ccc2oc(Cn3ccc4c(-c5ccccc5)cncc4c3=O)cc2c1 ; Cc1ccc2cc(Cn3ccc4c(-c5ccccc5)cncc4c3=O)[nH]c2c1 ; CC1CCC(CNCc2ccc3nc(Cn4ccc5c(-c6ccccc6)cncc5c4=O)cn3c2)CC1]

In some embodiments, the compound, or the pharmaceutically acceptable salt thereof, is selected from the group consisting of

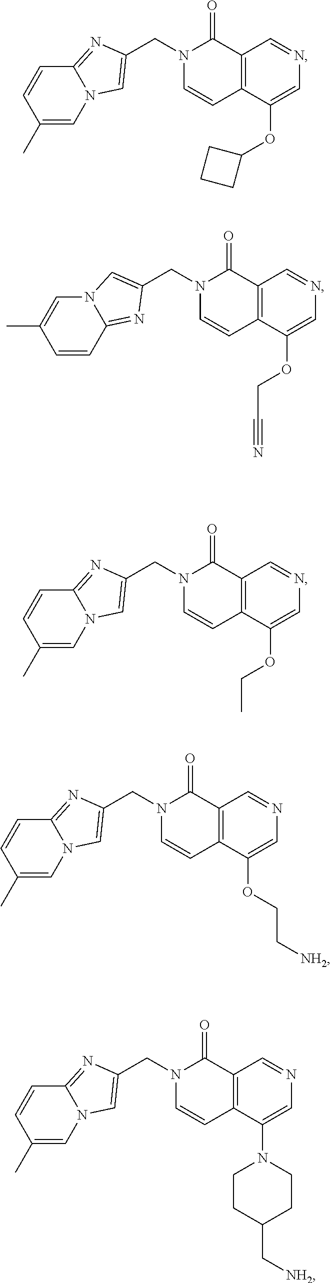 [SMILES: Cc1ccc2nc(Cn3ccc4c(OC5CCC5)cncc4c3=O)cn2c1 ; Cc1ccc2nc(Cn3ccc4c(OCC#N)cncc4c3=O)cn2c1 ; CCOc1cncc2c(=O)n(Cc3cn4cc(C)ccc4n3)ccc12 ; Cc1ccc2nc(Cn3ccc4c(OCCN)cncc4c3=O)cn2c1 ; Cc1ccc2nc(Cn3ccc4c(N5CCC(CN)CC5)cncc4c3=O)cn2c1]

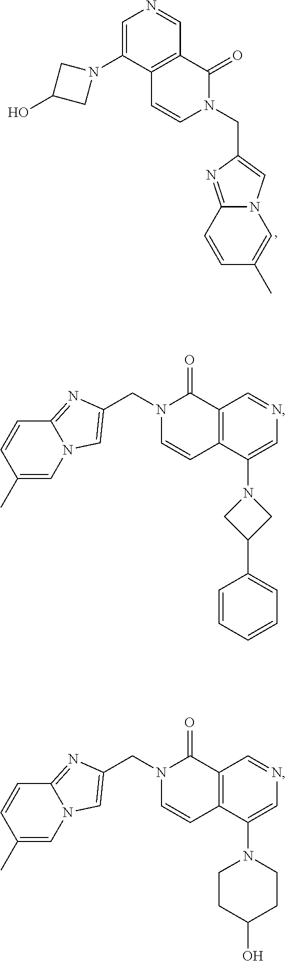 [SMILES: Cc1ccc2nc(Cn3ccc4c(N5CC(O)C5)cncc4c3=O)cn2c1 ; Cc1ccc2nc(Cn3ccc4c(N5CC(c6ccccc6)C5)cncc4c3=O)cn2c1 ; Cc1ccc2nc(Cn3ccc4c(N5CCC(O)CC5)cncc4c3=O)cn2c1]

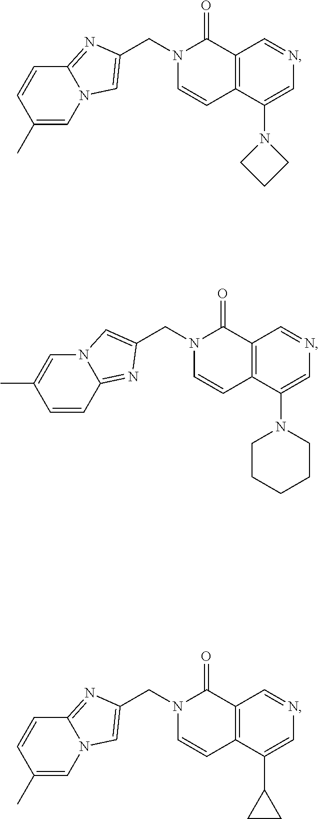 [SMILES: Cc1ccc2nc(Cn3ccc4c(N5CCC5)cncc4c3=O)cn2c1 ; Cc1ccc2nc(Cn3ccc4c(N5CCCCC5)cncc4c3=O)cn2c1 ; Cc1ccc2nc(Cn3ccc4c(C5CC5)cncc4c3=O)cn2c1]

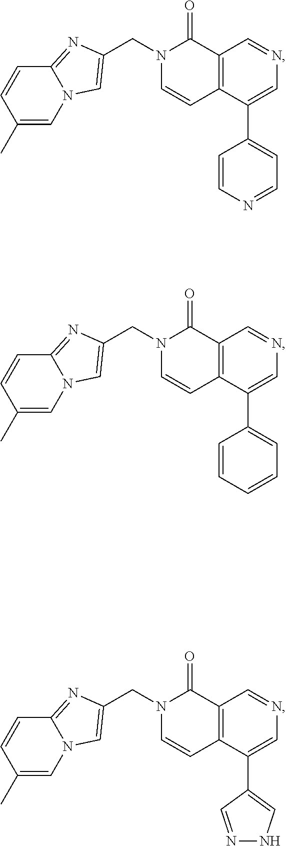 [SMILES: Cc1ccc2nc(Cn3ccc4c(-c5ccncc5)cncc4c3=O)cn2c1 ; Cc1ccc2nc(Cn3ccc4c(-c5ccccc5)cncc4c3=O)cn2c1 ; Cc1ccc2nc(Cn3ccc4c(-c5cn[nH]c5)cncc4c3=O)cn2c1]

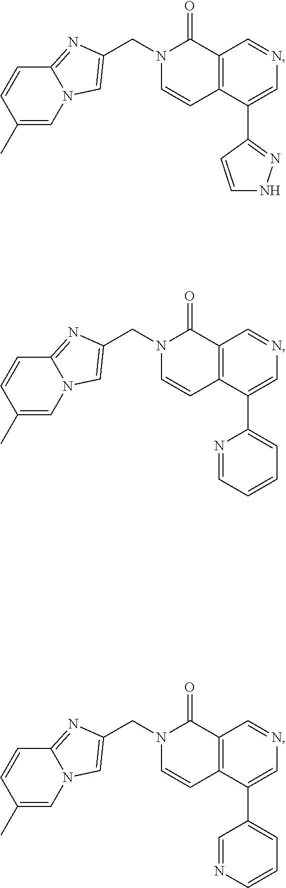 [SMILES: Cc1ccc2nc(Cn3ccc4c(-c5cc[nH]n5)cncc4c3=O)cn2c1 ; Cc1ccc2nc(Cn3ccc4c(-c5ccccn5)cncc4c3=O)cn2c1 ; Cc1ccc2nc(Cn3ccc4c(-c5cccnc5)cncc4c3=O)cn2c1]

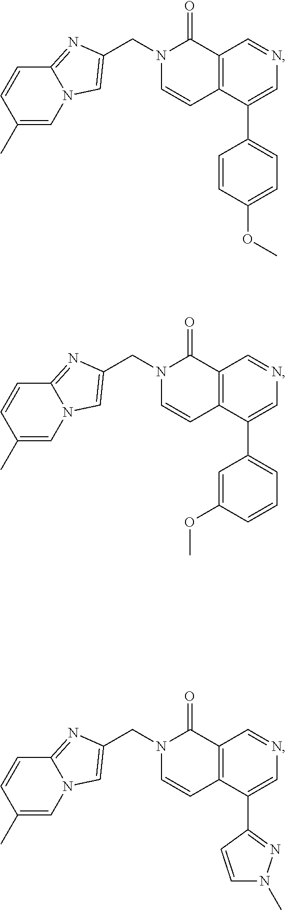 [SMILES: COc1ccc(-c2cncc3c(=O)n(Cc4cn5cc(C)ccc5n4)ccc23)cc1 ; COc1cccc(-c2cncc3c(=O)n(Cc4cn5cc(C)ccc5n4)ccc23)c1 ; Cc1ccc2nc(Cn3ccc4c(-c5ccn(C)n5)cncc4c3=O)cn2c1]

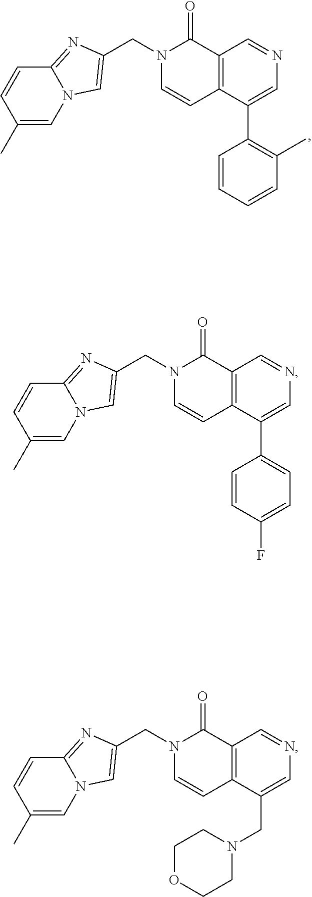 [SMILES: Cc1ccc2nc(Cn3ccc4c(-c5ccccc5C)cncc4c3=O)cn2c1 ; Cc1ccc2nc(Cn3ccc4c(-c5ccc(F)cc5)cncc4c3=O)cn2c1 ; Cc1ccc2nc(Cn3ccc4c(CN5CCOCC5)cncc4c3=O)cn2c1]

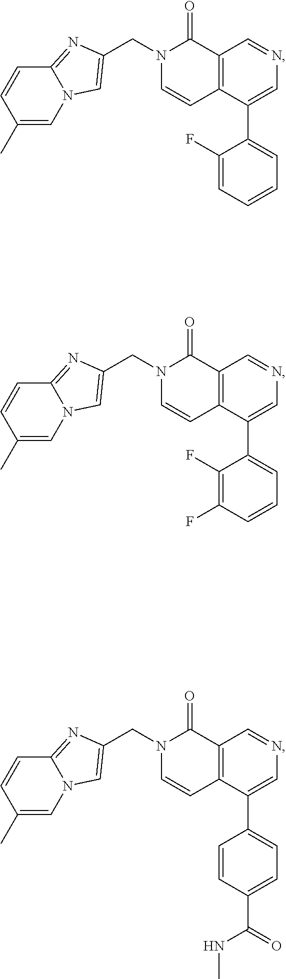 [SMILES: Cc1ccc2nc(Cn3ccc4c(-c5ccccc5F)cncc4c3=O)cn2c1 ; Cc1ccc2nc(Cn3ccc4c(-c5cccc(F)c5F)cncc4c3=O)cn2c1 ; CNC(=O)c1ccc(-c2cncc3c(=O)n(Cc4cn5cc(C)ccc5n4)ccc23)cc1]

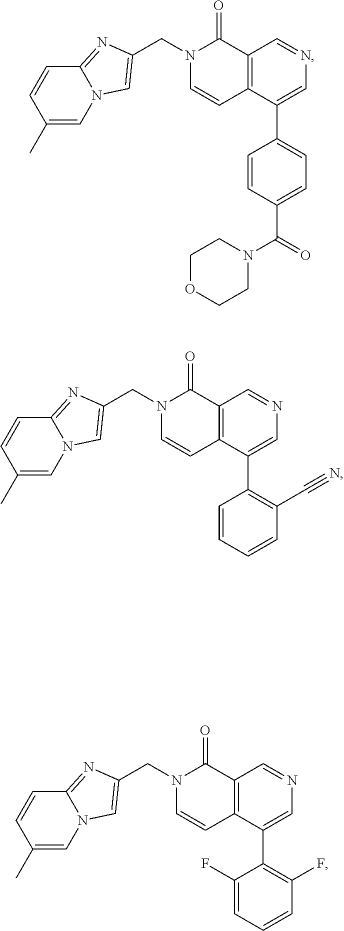 [SMILES: Cc1ccc2nc(Cn3ccc4c(-c5ccc(C(=O)N6CCOCC6)cc5)cncc4c3=O)cn2c1 ; Cc1ccc2nc(Cn3ccc4c(-c5ccccc5C#N)cncc4c3=O)cn2c1 ; Cc1ccc2nc(Cn3ccc4c(-c5c(F)cccc5F)cncc4c3=O)cn2c1]

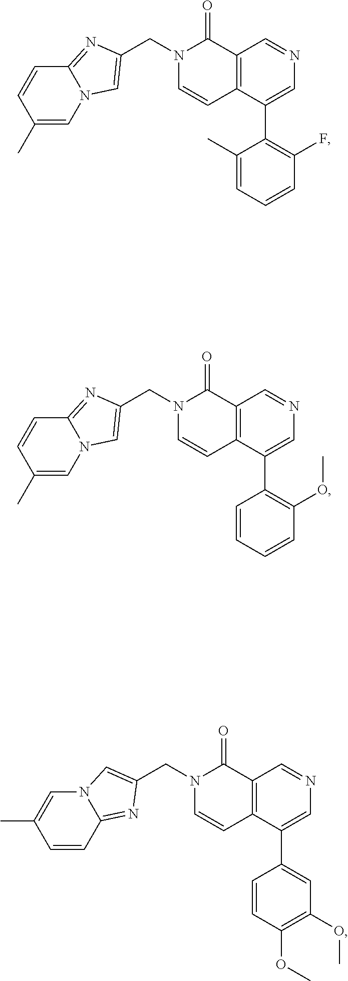

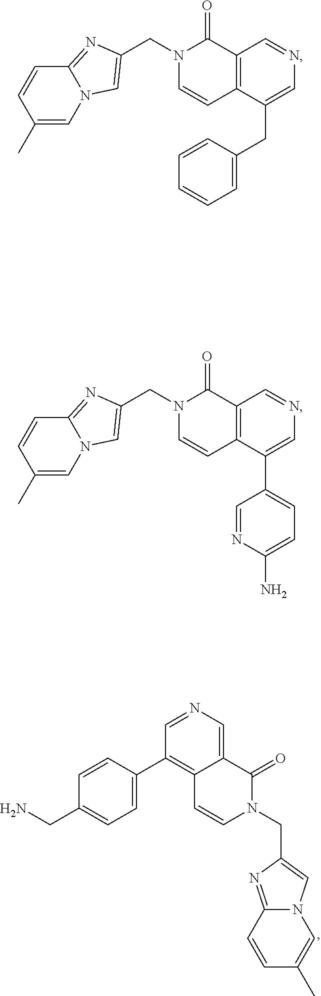 [SMILES: Cc1ccc2nc(Cn3ccc4c(Cc5ccccc5)cncc4c3=O)cn2c1 ; Cc1ccc2nc(Cn3ccc4c(-c5ccc(N)nc5)cncc4c3=O)cn2c1 ; Cc1ccc2nc(Cn3ccc4c(-c5ccc(CN)cc5)cncc4c3=O)cn2c1]

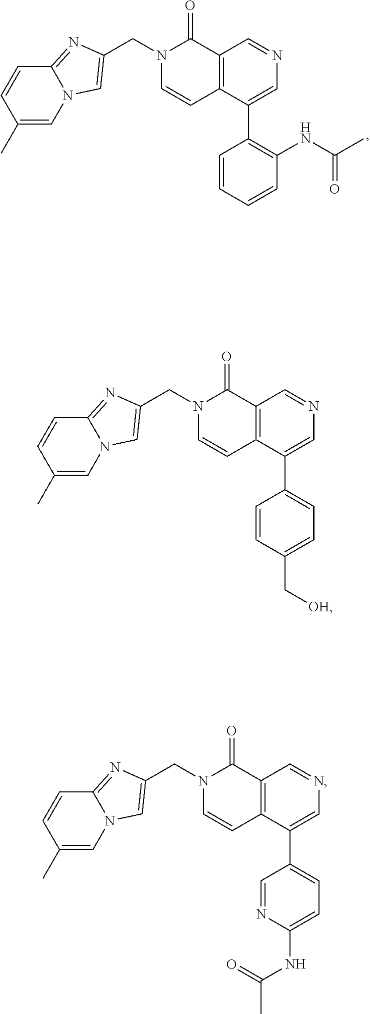 [SMILES: CC(=O)Nc1ccccc1-c1cncc2c(=O)n(Cc3cn4cc(C)ccc4n3)ccc12 ; Cc1ccc2nc(Cn3ccc4c(-c5ccc(CO)cc5)cncc4c3=O)cn2c1 ; CC(=O)Nc1ccc(-c2cncc3c(=O)n(Cc4cn5cc(C)ccc5n4)ccc23)cn1]

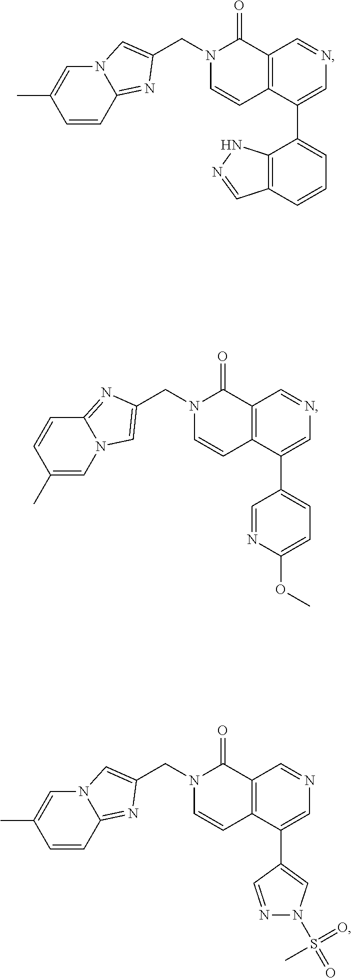

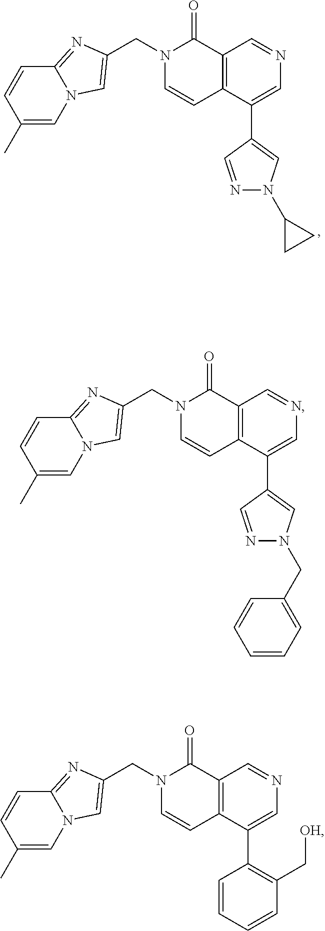 [SMILES: Cc1ccc2nc(Cn3ccc4c(-c5cnn(C6CC6)c5)cncc4c3=O)cn2c1 ; Cc1ccc2nc(Cn3ccc4c(-c5cnn(Cc6ccccc6)c5)cncc4c3=O)cn2c1 ; Cc1ccc2nc(Cn3ccc4c(-c5ccccc5CO)cncc4c3=O)cn2c1]

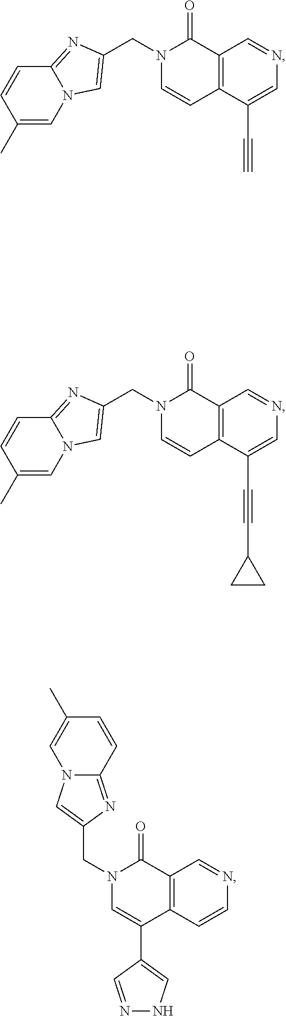 [SMILES: C#Cc1cncc2c(=O)n(Cc3cn4cc(C)ccc4n3)ccc12 ; Cc1ccc2nc(Cn3ccc4c(C#CC5CC5)cncc4c3=O)cn2c1 ; Cc1ccc2nc(Cn3cc(-c4cn[nH]c4)c4ccncc4c3=O)cn2c1]

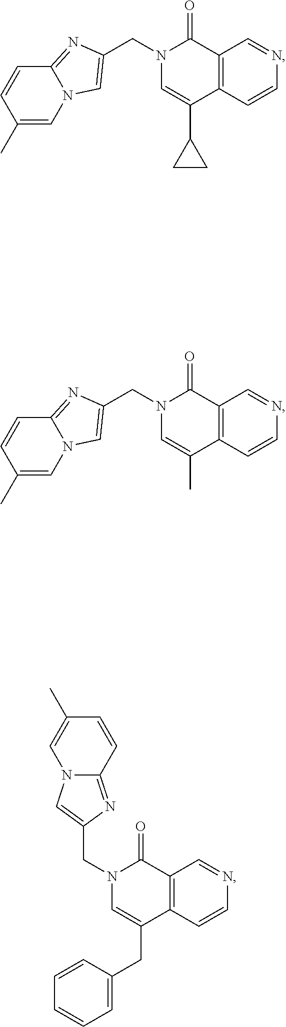 [SMILES: Cc1ccc2nc(Cn3cc(C4CC4)c4ccncc4c3=O)cn2c1 ; Cc1ccc2nc(Cn3cc(C)c4ccncc4c3=O)cn2c1 ; Cc1ccc2nc(Cn3cc(Cc4ccccc4)c4ccncc4c3=O)cn2c1]

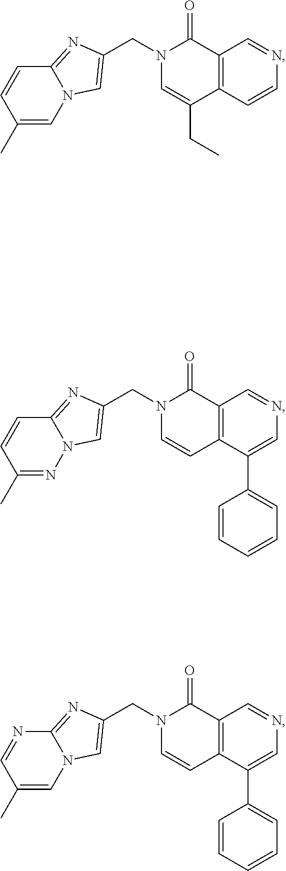 [SMILES: CCc1cn(Cc2cn3cc(C)ccc3n2)c(=O)c2cnccc12 ; Cc1ccc2nc(Cn3ccc4c(-c5ccccc5)cncc4c3=O)cn2n1 ; Cc1cnc2nc(Cn3ccc4c(-c5ccccc5)cncc4c3=O)cn2c1]

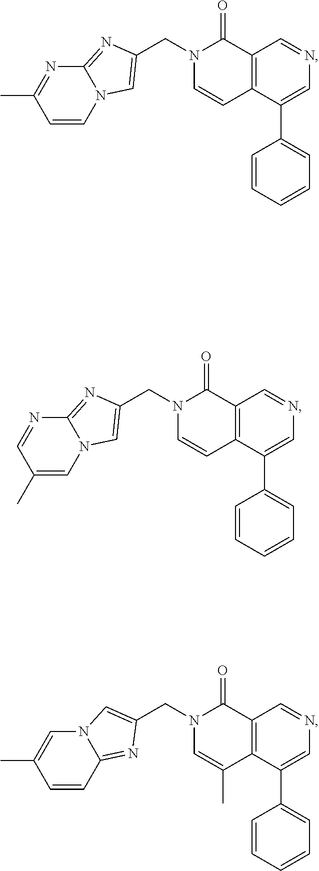 [SMILES: Cc1ccn2cc(Cn3ccc4c(-c5ccccc5)cncc4c3=O)nc2n1 ; Cc1cnc2nc(Cn3ccc4c(-c5ccccc5)cncc4c3=O)cn2c1 ; Cc1ccc2nc(Cn3cc(C)c4c(-c5ccccc5)cncc4c3=O)cn2c1]

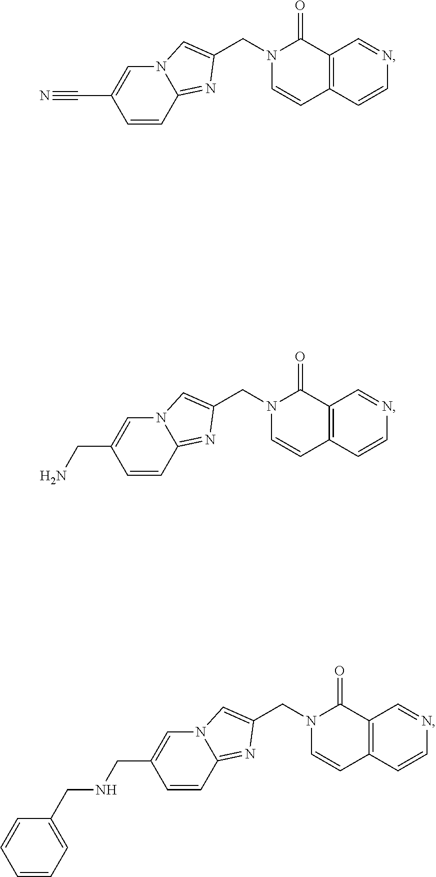 [SMILES: N#Cc1ccc2nc(Cn3ccc4ccncc4c3=O)cn2c1 ; NCc1ccc2nc(Cn3ccc4ccncc4c3=O)cn2c1 ; O=c1c2cnccc2ccn1Cc1cn2cc(CNCc3ccccc3)ccc2n1]

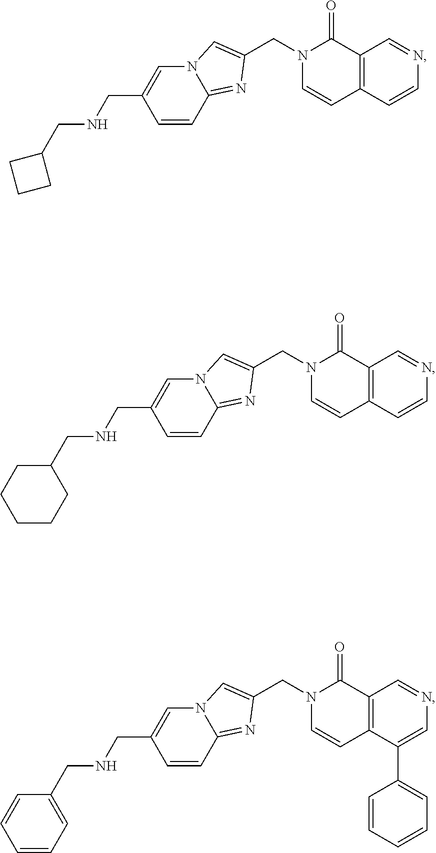 [SMILES: O=c1c2cnccc2ccn1Cc1cn2cc(CNCC3CCC3)ccc2n1 ; O=c1c2cnccc2ccn1Cc1cn2cc(CNCC3CCCCC3)ccc2n1 ; O=c1c2cncc(-c3ccccc3)c2ccn1Cc1cn2cc(CNCc3ccccc3)ccc2n1]

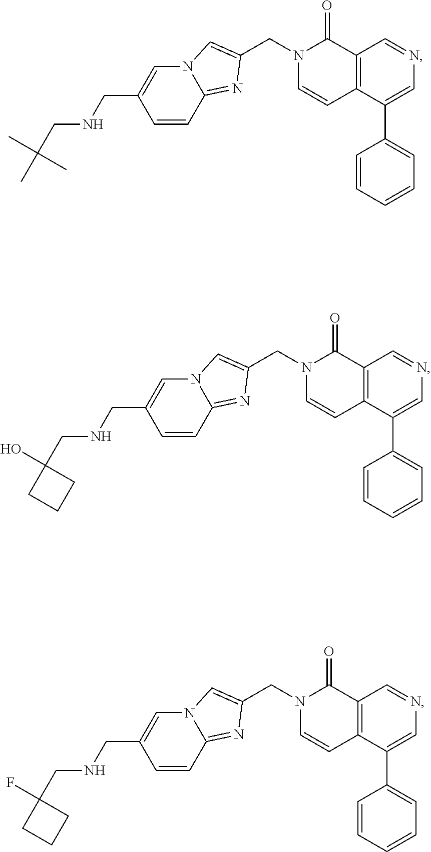 [SMILES: CC(C)(C)CNCc1ccc2nc(Cn3ccc4c(-c5ccccc5)cncc4c3=O)cn2c1 ; O=c1c2cncc(-c3ccccc3)c2ccn1Cc1cn2cc(CNCC3(O)CCC3)ccc2n1 ; O=c1c2cncc(-c3ccccc3)c2ccn1Cc1cn2cc(CNCC3(F)CCC3)ccc2n1]

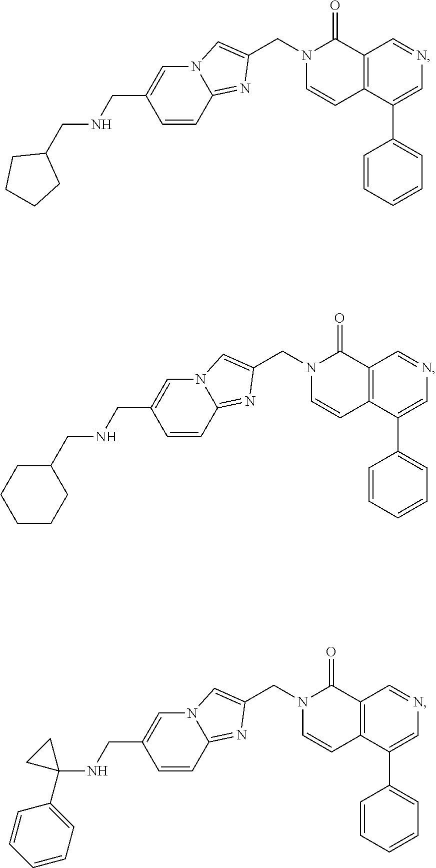 [SMILES: O=c1c2cncc(-c3ccccc3)c2ccn1Cc1cn2cc(CNCC3CCCC3)ccc2n1 ; O=c1c2cncc(-c3ccccc3)c2ccn1Cc1cn2cc(CNCC3CCCCC3)ccc2n1 ; O=c1c2cncc(-c3ccccc3)c2ccn1Cc1cn2cc(CNC3(c4ccccc4)CC3)ccc2n1]

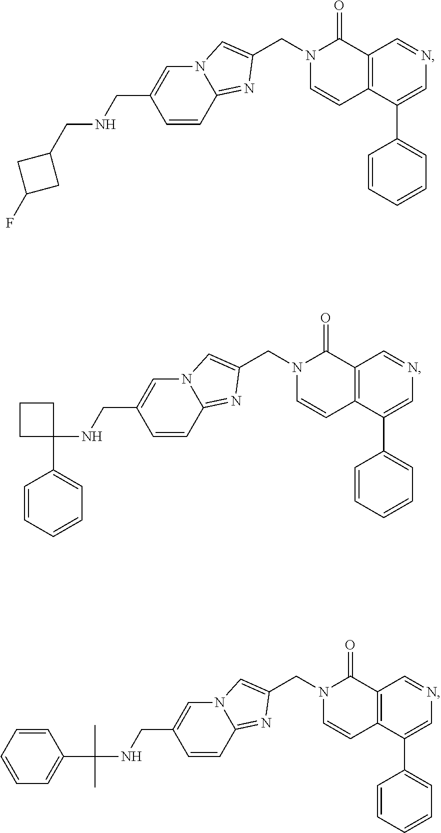 [SMILES: O=c1c2cncc(-c3ccccc3)c2ccn1Cc1cn2cc(CNCC3CC(F)C3)ccc2n1 ; O=c1c2cncc(-c3ccccc3)c2ccn1Cc1cn2cc(CNC3(c4ccccc4)CCC3)ccc2n1 ; CC(C)(NCc1ccc2nc(Cn3ccc4c(-c5ccccc5)cncc4c3=O)cn2c1)c1ccccc1]

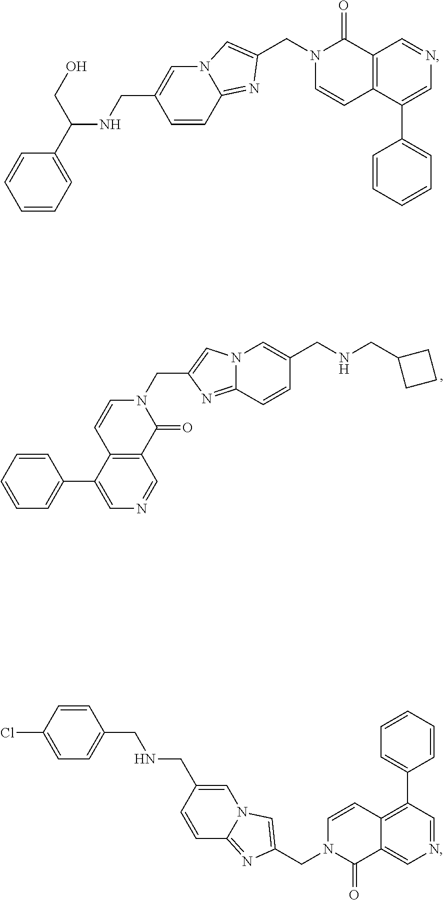 [SMILES: O=c1c2cncc(-c3ccccc3)c2ccn1Cc1cn2cc(CNC(CO)c3ccccc3)ccc2n1 ; O=c1c2cncc(-c3ccccc3)c2ccn1Cc1cn2cc(CNCC3CCC3)ccc2n1 ; O=c1c2cncc(-c3ccccc3)c2ccn1Cc1cn2cc(CNCc3ccc(Cl)cc3)ccc2n1]

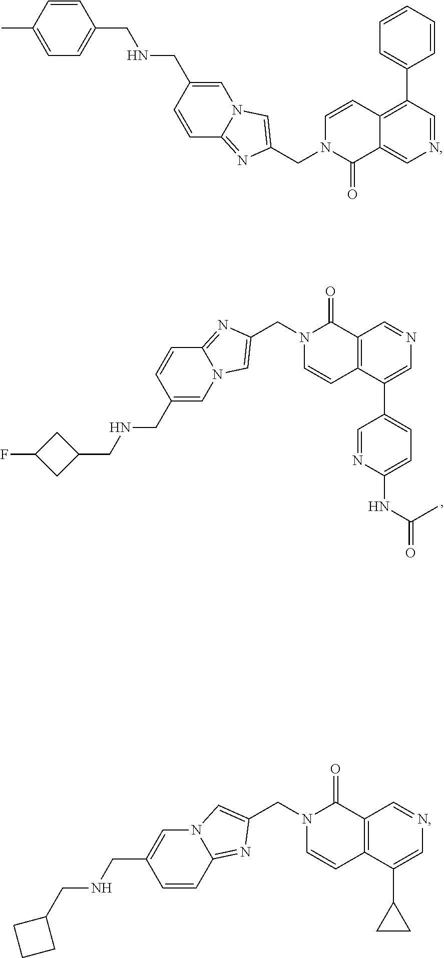 [SMILES: Cc1ccc(CNCc2ccc3nc(Cn4ccc5c(-c6ccccc6)cncc5c4=O)cn3c2)cc1 ; CC(=O)Nc1ccc(-c2cncc3c(=O)n(Cc4cn5cc(CNCC6CC(F)C6)ccc5n4)ccc23)cn1 ; O=c1c2cncc(C3CC3)c2ccn1Cc1cn2cc(CNCC3CCC3)ccc2n1]

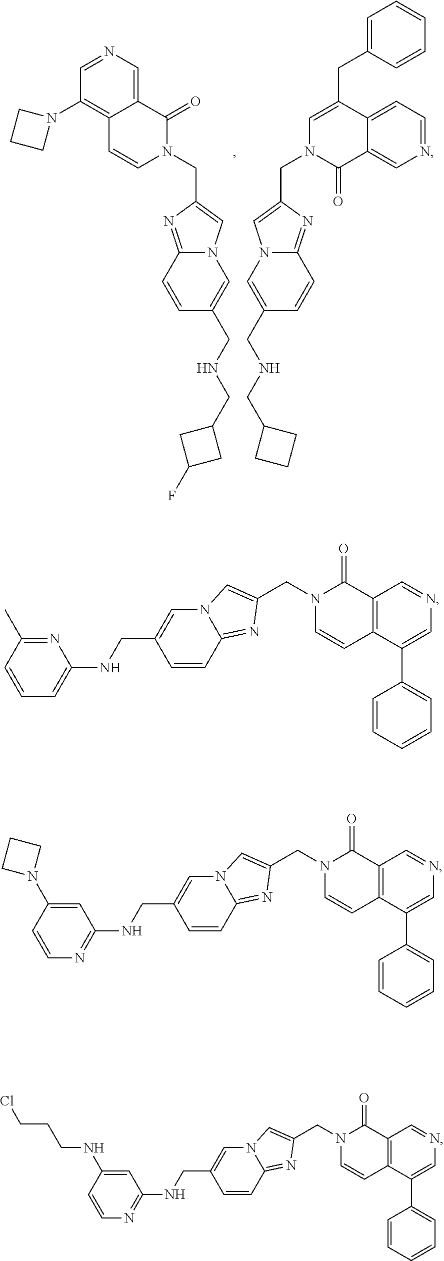

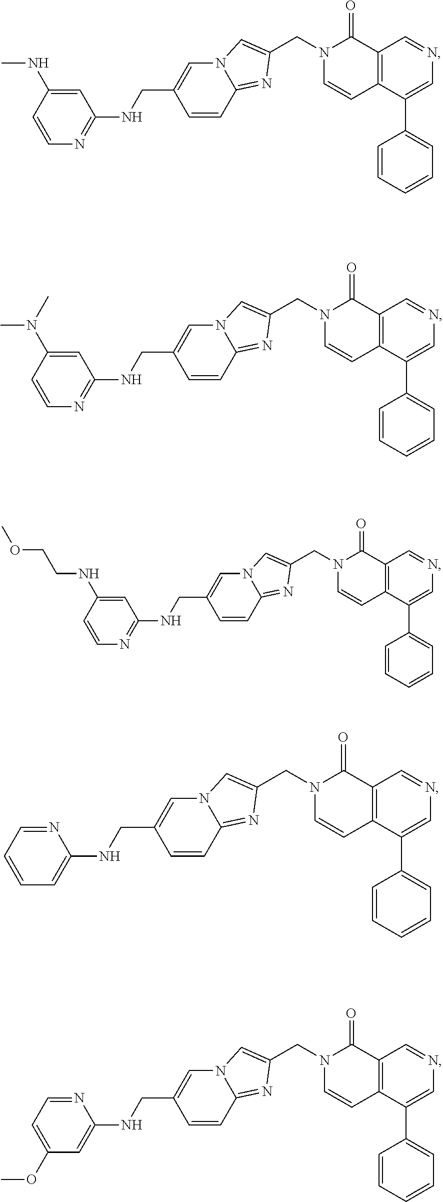 [SMILES: CNc1ccnc(NCc2ccc3nc(Cn4ccc5c(-c6ccccc6)cncc5c4=O)cn3c2)c1 ; CN(C)c1ccnc(NCc2ccc3nc(Cn4ccc5c(-c6ccccc6)cncc5c4=O)cn3c2)c1 ; COCCNc1ccnc(NCc2ccc3nc(Cn4ccc5c(-c6ccccc6)cncc5c4=O)cn3c2)c1 ; O=c1c2cncc(-c3ccccc3)c2ccn1Cc1cn2cc(CNc3ccccn3)ccc2n1 ; COc1ccnc(NCc2ccc3nc(Cn4ccc5c(-c6ccccc6)cncc5c4=O)cn3c2)c1]

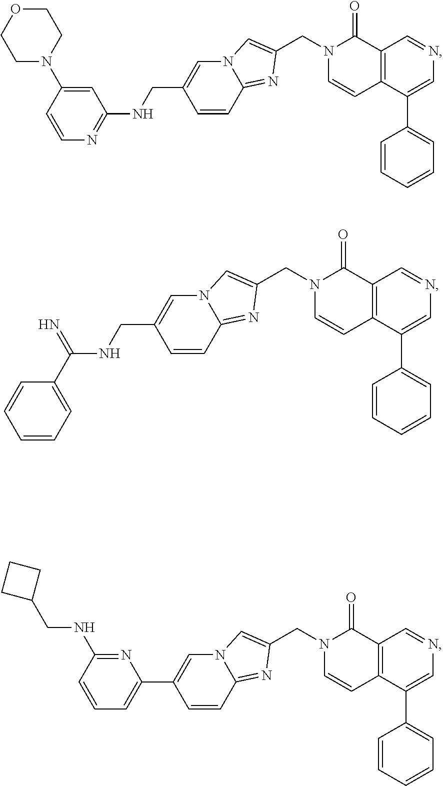 [SMILES: O=c1c2cncc(-c3ccccc3)c2ccn1Cc1cn2cc(CNc3cc(N4CCOCC4)ccn3)ccc2n1 ; N=C(NCc1ccc2nc(Cn3ccc4c(-c5ccccc5)cncc4c3=O)cn2c1)c1ccccc1 ; O=c1c2cncc(-c3ccccc3)c2ccn1Cc1cn2cc(-c3cccc(NCC4CCC4)n3)ccc2n1]

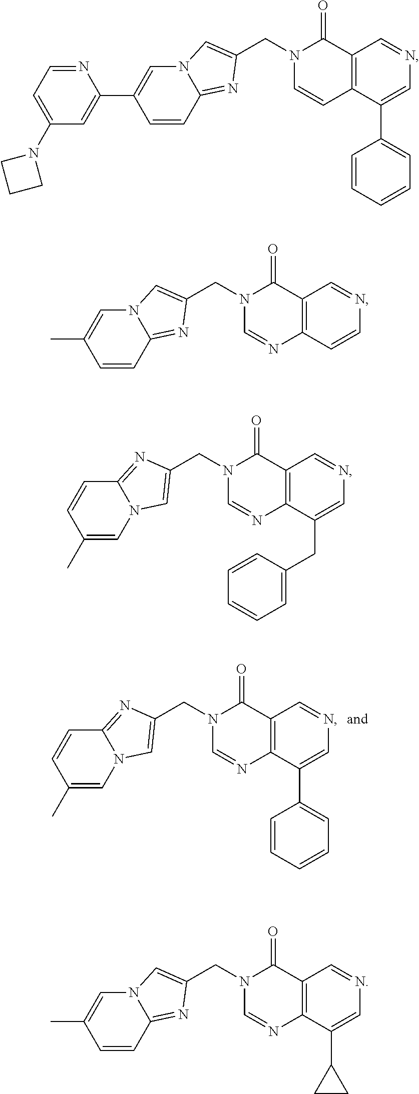 [SMILES: O=c1c2cncc(-c3ccccc3)c2ccn1Cc1cn2cc(-c3cc(N4CCC4)ccn3)ccc2n1 ; Cc1ccc2nc(Cn3cnc4ccncc4c3=O)cn2c1 ; Cc1ccc2nc(Cn3cnc4c(Cc5ccccc5)cncc4c3=O)cn2c1 ; Cc1ccc2nc(Cn3cnc4c(-c5ccccc5)cncc4c3=O)cn2c1 ; Cc1ccc2nc(Cn3cnc4c(C5CC5)cncc4c3=O)cn2c1]

In some embodiments, the compound, or the pharmaceutically acceptable salt thereof, is selected from the group consisting of

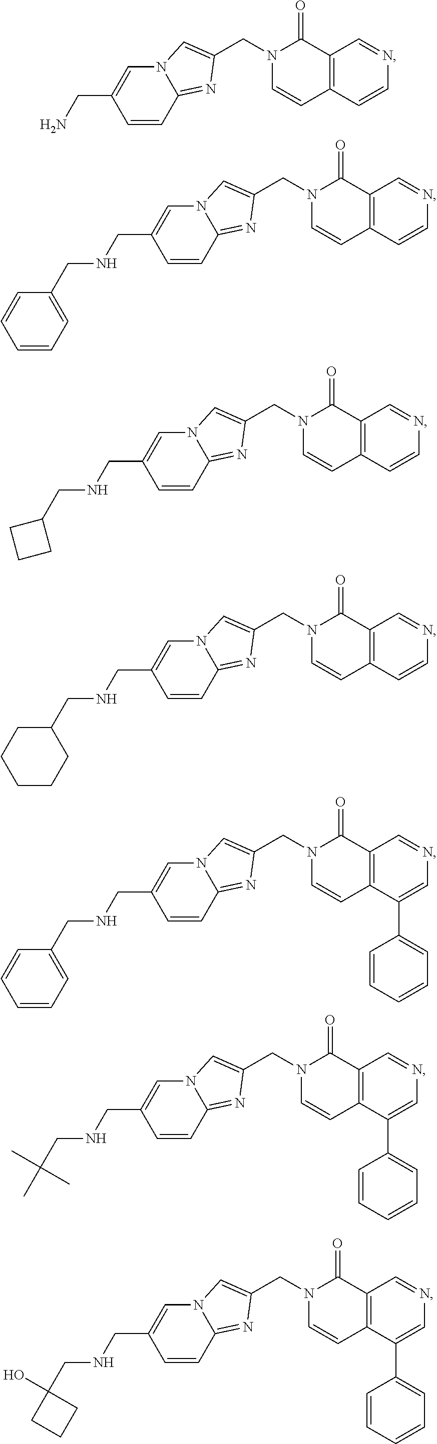

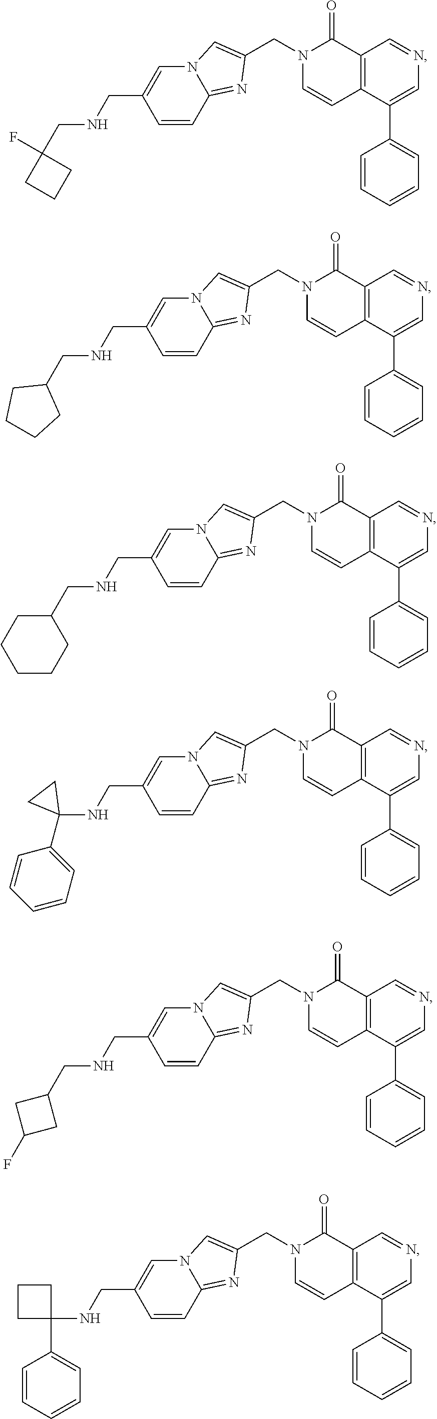 [SMILES: O=c1c2cncc(-c3ccccc3)c2ccn1Cc1cn2cc(CNCC3(F)CCC3)ccc2n1 ; O=c1c2cncc(-c3ccccc3)c2ccn1Cc1cn2cc(CNCC3CCCC3)ccc2n1 ; O=c1c2cncc(-c3ccccc3)c2ccn1Cc1cn2cc(CNCC3CCCCC3)ccc2n1 ; O=c1c2cncc(-c3ccccc3)c2ccn1Cc1cn2cc(CNC3(c4ccccc4)CC3)ccc2n1 ; O=c1c2cncc(-c3ccccc3)c2ccn1Cc1cn2cc(CNCC3CC(F)C3)ccc2n1 ; O=c1c2cncc(-c3ccccc3)c2ccn1Cc1cn2cc(CNC3(c4ccccc4)CCC3)ccc2n1]

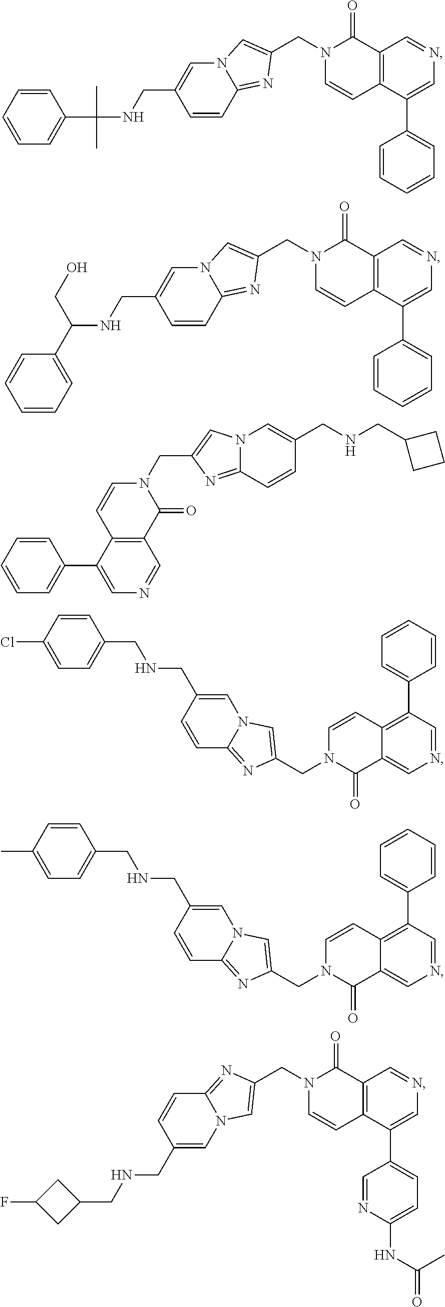

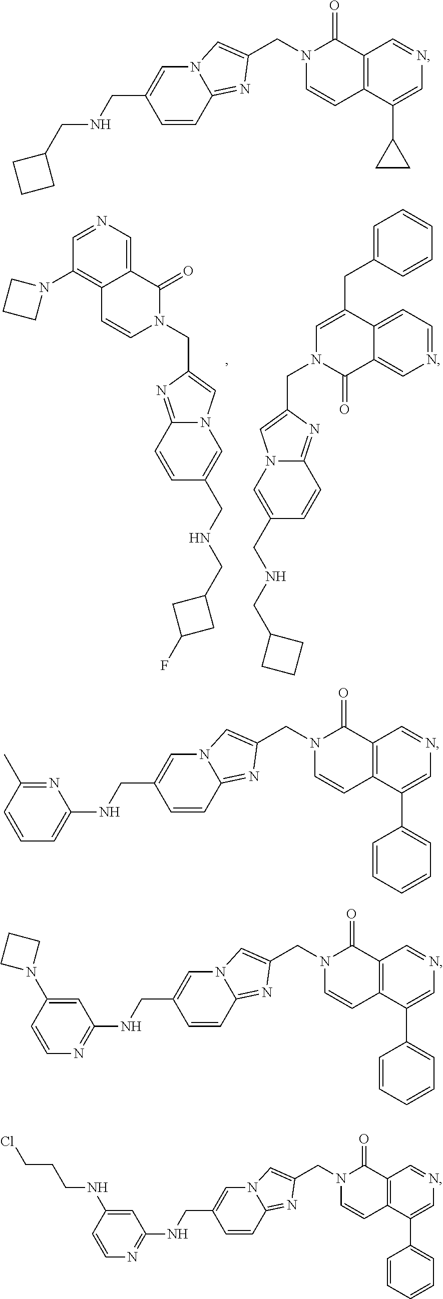

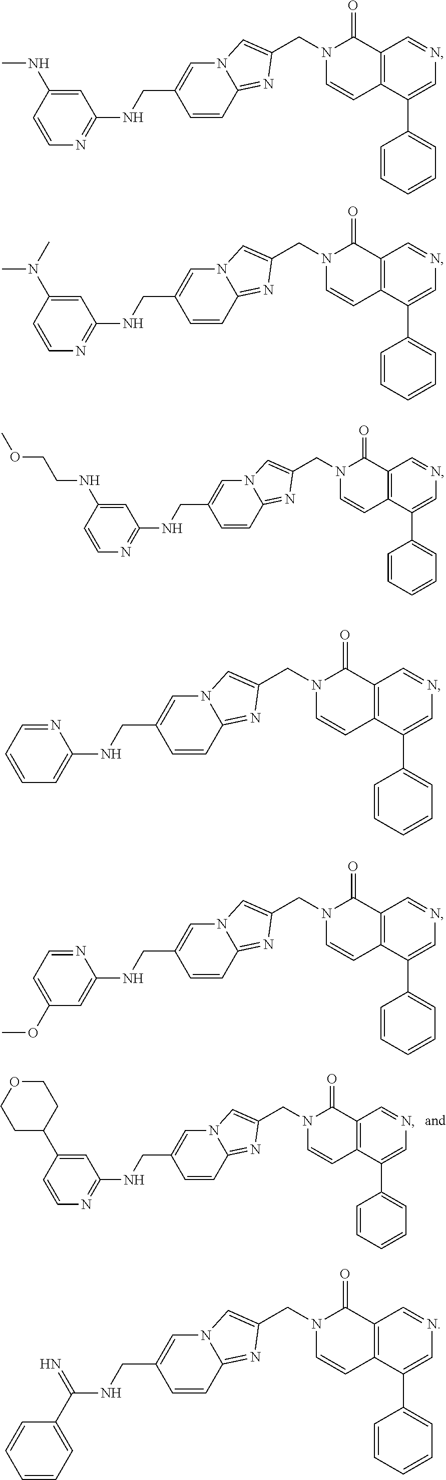

In some embodiments, the compound or the pharmaceutically acceptable salt thereof is of the structure

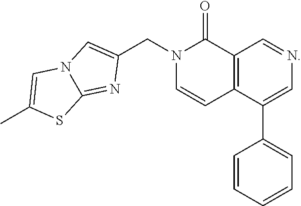 [SMILES: Cc1cn2cc(Cn3ccc4c(-c5ccccc5)cncc4c3=O)nc2s1]

In some embodiments, the compound, or the pharmaceutically acceptable salt thereof is selected from the group consisting of

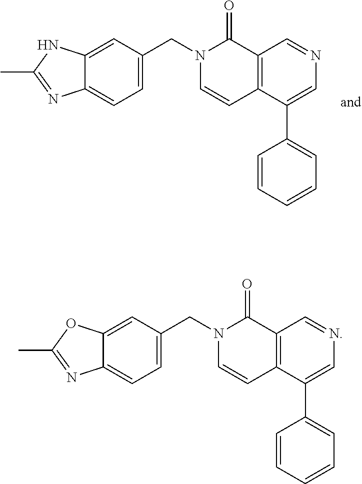 [SMILES: Cc1nc2ccc(Cn3ccc4c(-c5ccccc5)cncc4c3=O)cc2[nH]1 ; Cc1nc2ccc(Cn3ccc4c(-c5ccccc5)cncc4c3=O)cc2o1]

In some embodiments, the compound, or the pharmaceutically acceptable salt thereof, is selected from the group consisting of

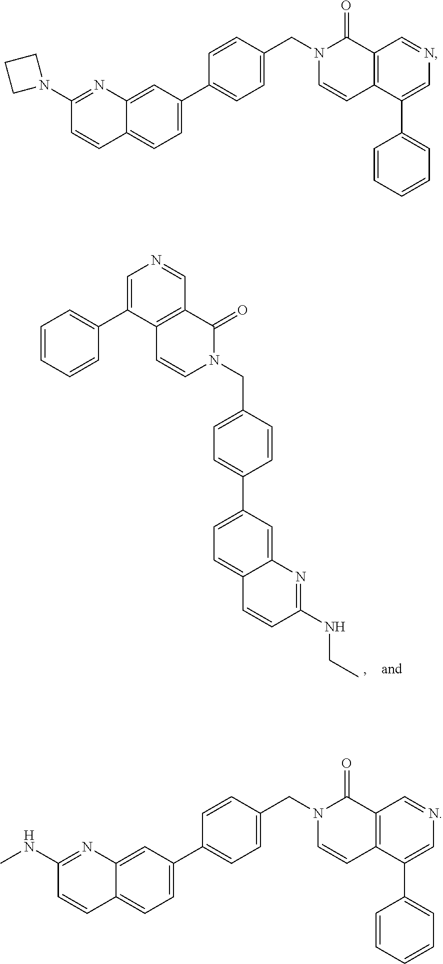

In some embodiments, the compound, or the pharmaceutically acceptable salt thereof, is selected from the group consisting of:
5-{6,6-difluoro-2-azaspiro[3.3]heptan-2-yl}-2-({6-[(4,4-dimethylpiperidin-1-yl)methyl]imidazo[1,2-a]pyridin-2-yl}methyl)-1,2-dihydro-2,7-naphthyridin-1-one;
5-(4,4-difluoropiperidin-1-yl)-2-({6-[(4,4-dimethylpiperidin-1-yl)methyl]imidazo[1,2-a]pyridin-2-yl}methyl)-1,2-dihydro-2,7-naphthyridin-1-one;
2-({6-[(4,4-dimethylpiperidin-1-yl}methyl]imidazo[1,2-a]pyridin-2-yl)methyl)-5-(morpholin-4-yl)-1,2-dihydro-2,7-naphthyridin-1-one;
2-({6-[(4,4-dimethylpiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one;
5-((1 S,4S)-2-oxa-5-azabicyclo[2.2.1]heptan-5-yl)-2-((6-((4,4-dimethylpiperidin-1-yl)methyl)imidazo[1,2-a]pyridin-2-yl)methyl)-2,7-naphthyridin-1(2H)-one;
2-({6-[(4,4-dimethylpiperidin-1-yl)methyl]imidazo[1,2-a]pyridin-2-yl}methyl)-5-[(1R,4R)-2-oxa-5-azabicyclo[2.2.1]heptan-5-yl]-1,2-dihydro-2,7-naphthyridin-1-one;
2-({6-[(4,4-dimethylpiperidin-1-yl)methyl]imidazo[1,2-a]pyridin-2-yl}methyl)-5-{hexahydro-1H-furo[3,4-c]pyrrol-5-yl}-1,2-dihydro-2,7-naphthyridin-1-one;
2-({6-[(4,4-dimethylpiperidin-1-yl)methyl]imidazo[1,2-a]pyridin-2-yl}methyl)-5-[3-(morpholin-4-yl)azetidin-1-yl]-1,2-dihydro-2,7-naphthyridin-1-one;
   5-{2,7-diazaspiro[3.5]nonan-2-yl}-2-({6-[(4,4-dimethylpiperidin-1-yl)methyl]imidazo[1,2-a]pyridin-2-yl}methyl)-1,2-dihydro-2,7-naphthyridin-1-one;
5-(3,4-dihydro-2H-1,4-benzoxazin-4-yl)-2-({6-[(4,4-dimethylpiperidin-1-yl)methyl]imidazo[1,2-a]pyridin-2-yl}methyl)-1,2-dihydro-2,7-naphthyridin-1-one;
2-({6-[(4,4-dimethylpiperidin-1-yl)methyl]imidazo[1,2-a]pyridin-2-yl}methyl)-5-phenyl-1,2-dihydro-2,7-naphthyridin-1-one;
2-({6-[(4,4-dimethylpiperidin-1-yl)methyl]imidazo[1,2-a]pyridin-2-yl}methyl)-5-(quinolin-5-yl)-1,2-dihydro-2,7-naphthyridin-1-one;
   4-[7-({6-[(4,4-dimethylpiperidin-1-yl)methyl]imidazo[1,2-a]pyridin-2-yl}methyl)-8-oxo-7,8-dihydro-2,7-naphthyridin-4-yl]benzene-1-sulfonamide;
2-(( 6-((4,4-dimethylpiperidin-1-yl)methyl)imidazo[1,2-a]pyridin-2-yl)methyl)-5-(pyridin-4-ylethynyl)-2,7-naphthyridin-1(2H)-one;
   5-{3-[(dimethylamino)methyl]azetidin-1-yl}-2-({6-[(4,4-dimethylpiperidin-1-yl)methyl]imidazo[1,2-a]pyridin-2-yl}methyl)-1,2-dihydro-2,7-naphthyridin-1-one;
2-({6-[(4,4-dimethylpiperidin-1-yl)methyl]imidazo[1,2-a]pyridin-2-yl}methyl)-5-[3-(4-methylpiperazin-1-yl)azetidin-1-yl]-1,2-dihydro-2,7-naphthyridin-1-one;
   2-{[6-({6-azaspiro[2.5]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
   2-{[6-({2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-((6-((7-azaspiro[3.5]nonan-7-yl)methyl)imidazo[1,2-a]pyridin-2-yl)methyl)-5-(7-oxa-2-azaspiro[3.5]nonan-2-yl)-2,7-naphthyridin-1(2H)-one;
   5-{7-oxa-2-azaspiro[3.5]nonan-2-yl}-2-{[6-({2-oxa-7-azaspiro[4.4]nonan-7-yl}methyl)imidazo[1,2-a]pyridin-2-yl]methyl}-1,2-dihydro-2,7-naphthyridin-1-one;
   5-{7-oxa-2-azaspiro[3.5]nonan-2-yl}-2-{[6-({6-oxa-3-azabicyclo[3.1.1]heptan-3-yl}methyl)imidazo[1,2-a]pyridin-2-yl]methyl}-1,2-dihydro-2,7-naphthyridin-1-one;
   5-{7-oxa-2-azaspiro[3.5]nonan-2-yl}-2-[(6-{[4-(trifluoromethyl)piperidin-1-yl]methyl}imidazo[1,2-a]pyridin-2-yl)methyl]-1,2-dihydro-2,7-naphthyridin-1-one;
   2-{[6-({8-methyl-3,8-diazabicyclo[3.2.1]octan-3-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
4-methyl-1-({2-[(5-{7-oxa-2-azaspiro[3.5]nonan-2-yl}-1-oxo-1,2-dihydro-2,7-naphthyridin-2-yl)methyl]imidazo[1,2-a]pyridin-6-yl}methyl)piperidine-4-carbonitrile;
2-{[6-({6,6-difluoro-2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-{[6-({6-fluoro-2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-{[6-({2-azaspiro[4.4]nonan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-({6-[(4-methylpiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one;
2-({6-[(4,4-difluoropiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one;
2-{[6-({2,2-difluoro-6-azaspiro[3.4]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-{[6-({1,1-difluoro-6-azaspiro[2.5]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
5-{6,6-difluoro-2-azaspiro[3.3]heptan-2-yl}-2-{[6-({[(3-fluorocyclobutyl)methyl]amino}methyl)imidazo[1,2-a]pyridin-2-yl]methyl}-1,2-dihydro-2,7-naphthyridin-1-one;
   2-{[6-({[(3-fluorocyclobutyl)methyl]amino}methyl)imidazo[1,2-a]pyridin-2-yl]methyl}-5-{2-oxa-6-azaspiro[3.3]heptan-6-yl}-1,2-dihydro-2,7-naphthyridin-1-one;
   2-{[6-({[(3-fluorocyclobutyl)methyl]amino}methyl)imidazo[1,2-a]pyridin-2-yl]methyl}-5-{7-oxa-2-azaspiro[3.5]nonan-2-yl}-1,2-dihydro-2,7-naphthyridin-1-one;
   2-{[6-({[(3-fluorocyclobutyl)methyl]amino}methyl)imidazo[1,2-a]pyridin-2-yl]methyl}-5-{6-oxa-2-azaspiro[3.5]nonan-2-yl}-1,2-dihydro-2,7-naphthyridin-1-one;
   5-{7,7-difluoro-2-azaspiro[3.5]nonan-2-yl}-2-{[6-({[(3-fluorocyclobutyl)methyl]amino}methyl)imidazo[1,2-a]pyridin-2-yl]methyl}-1,2-dihydro-2,7-naphthyridin-1-one;
   5-{2,2-difluoro-6-azaspiro[3.4]octan-6-yl}-2-{[6-({[(3-fluorocyclobutyl)methyl]amino}methyl)imidazo[1,2-a]pyridin-2-yl]methyl}-1,2-dihydro-2,7-naphthyridin-1-one;
   2-{[6-({[(3-fluorocyclobutyl)methyl]amino}methyl)imidazo[1,2-a]pyridin-2-yl]methyl}-5-[3-(4-fluoropiperidin-1-yl)azetidin-1-yl]-1,2-dihydro-2,7-naphthyridin-1-one;
   2-{[6-({[(3-fluorocyclobutyl)methyl]amino}methyl)imidazo[1,2-a]pyridin-2-yl]methyl}-5-[3-(pyridin-4-yl)azetidin-1-yl]-1,2-dihydro-2,7-naphthyridin-1-one;
   2-{[6-({[(3-fluorocyclobutyl)methyl]amino}methyl)imidazo[1,2-a]pyridin-2-yl]methyl}-5-[3-(4-fluorophenyl)azetidin-1-yl]-1,2-dihydro-2,7-naphthyridin-1-one;
2-((6-((((3-fluorobicyclo[1.1.1]pentan-1-yl)methyl)amino)methyl)imidazo[1,2-a]pyridin-2-yl)methyl)-5-(6-oxa-2-azaspiro[3.4]octan-2-yl)-2,7-naphthyridin-1(2H)-one;
   2-[(6-{[({3-fluorobicyclo[1.1.1]pentan-1-yl})methyl)amino]methyl})imidazo[1,2-a]pyridin-2-yl)methyl]-5-{7-oxa-2-azaspiro[3.5]nonan-2-yl}-1,2-dihydro-2,7-naphthyridin-1-one;
   2-[(6-{[(({3-fluorobicyclo[1.1.1]pentan-1-yl}methyl)amino]methyl}imidazo[1,2-a]pyridin-2-yl)methyl]-5-{6-oxa-2-azaspiro[3.5]nonan-2-yl}-1,2-dihydro-2,7-naphthyridin-1-one;
   2-[(6-{[((3-fluorobicyclo[1.1.1]pentan-1-yl}methyl)amino]methyl)imidazo[1,2-a]pyridin-2-yl)methyl]-5-(2-fluorophenyl)-1,2-dihydro-2,7-naphthyridin-1-one;
   4-{7-[(6-{[((3-fluorobicyclo[1.1.1]pentan-1-yl)methyl)amino]methyl}imidazo[1,2-a]pyridin-2-yl)methyl]-8-oxo-7,8-dihydro-2,7-naphthyridin-4-yl}benzamide;
5-cyclobutyl-2-{[6-(([{3-fluorocyclobutyl)methyl]amino}methyl)imidazo[1,2-a]pyridin-2-yl]methyl}-1,2-dihydro-2,7-naphthyridin-1-one; and
   3-[(6-{[((3-fluorobicyclo[1.1.1]pentan-1-yl)methyl)amino]methyl}imidazo[1,2-a]pyridin-2-yl)methyl]-8-{2-oxa-6-azaspiro[3.3]heptan-6-yl}pyrido[3,4-d]pyridazin-4-one.

Unless otherwise stated, structures depicted herein are also meant to include salts (e.g. pharmaceutically acceptable salts), solvates, hydrates, and isomers (e.g. stereoisomers) thereof. Accordingly, the present disclosure is directed to compounds of Formulae I′, I, II, IIa, IIb, IIc, III, IIIa, IIIb, IIIc, IV, IVa, IVb, IVc, V, Va, Vb, VI, VIa, and VIb and salts, solvates, hydrates, and isomers thereof. Moreover, reference to compounds of Formulae I′, I, II, IIa, IIb, IIc, III, IIIa, IIIb, IIIc, IV, IVa, IVb, IVc, V, Va, Vb, VI, VIa, and VIb and pharmaceutically acceptable salts thereof is considered to include reference to solvates, hydrates, and isomers (e.g. stereoisomers) of any thereof.

In some embodiments, the compound is a solvate, hydrate, or isomer (e.g. stereoisomers) of a compound of Formulae I′, II″, I, II, IIa, IIb, IIc, III, IIIa, IIIb, IIIc, IV, IVa, IVb, IVc, V, Va, Vb, VI, VIa, VIb, and VII or a pharmaceutically acceptable salt thereof.

Unless otherwise stated, structures depicted herein are also meant to include compounds which differ only in the presence of one or more isotopically enriched atoms. For example, compounds having the present structure except for the replacement of a hydrogen atom by deuterium or tritium, the replacement of a carbon atom by ¹³C or ¹⁴C, the replacement of a nitrogen atom by ¹³N, or the replacement of an oxygen atom by ¹⁷O or ¹⁸O are within the scope of the present disclosure. Such isotopically labeled compounds are useful as research or diagnostic tools.

### Methods of Synthesizing the Compounds

The compounds of Formulae I′, II″, I, II, IIa, IIb, IIc, III, IIIa, IIIb, IIIc, IV, IVa, IVb, IVc, V, Va, Vb, VI, VIa, VIb, and VII and pharmaceutically acceptable salts thereof may be prepared by methods known in the art of organic synthesis as set forth in part by the following synthetic scheme. In the scheme described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999, which is hereby incorporated by reference in its entirety). These groups are removed at a convenient stage of the compound synthesis using methods that are readily apparent to those skilled in the art. The selection processes, as well as the reaction conditions and order of their execution, shall be consistent with the preparation of the disclosed compounds (e.g., Formulae I′, II″, I, II, IIa, IIb, IIc, III, IIIa, IIIb, IIIc, IV, IVa, IVb, IVc, V, Va, Vb, VI, VIa, VIb, and VII and pharmaceutically acceptable salts thereof).

Those skilled in the art will recognize if a stereocenter exists in the compounds disclosed compounds (e.g., Formulae I′, II″, I, II, IIa, IIb, IIc, III, IIIa, IIIb, IIIc, IV, IVa, IVb, IVc, V, Va, Vb, VI, VIa, VIb, and VII and pharmaceutically acceptable salts thereof). Accordingly, the present disclosure includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994), which is hereby incorporated by reference in its entirety.

The compounds described herein may be made from commercially available starting materials or synthesized using known organic, inorganic, and/or enzymatic processes.

Compounds of Formulae I′, II″, I, II, IIa, IIb, IIc, III, IIIa, IIIb, IIIc, IV, IVa, IVb, IVc, V, Va, Vb, VI, VIa, VIb, and VII can be prepared according to procedures outlined in Schemes and Examples herein. In the Examples section, compounds of the present disclosure are further exemplified by specific examples. Unless otherwise specified, all temperatures were experessed in ° C. and all reactions are conducted at room temperature.

### Pharmaceutical Compositions

The compounds of Formulae I′, II″, I, II, IIa, IIb, IIc, III, IIIa, IIIb, IIIc, IV, IVa, IVb, IVc, V, Va, Vb, VI, VIa, VIb, and VII and pharmaceutically acceptable salts thereof may be used on their own but will generally be administered in the form of a pharmaceutical composition in which the disclosed compound or pharmaceutically acceptable salts thereof is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988, which is hereby incorporated by reference in its entirety.

The present disclosure also provides a pharmaceutical composition comprising a compound of Formulae I′, II″, I, II, IIa, IIb, IIc, III, IIIa, IIIb, IIIc, IV, IVa, IVb, IVc, V, Va, Vb, VI, VIa, VIb, and VII, or a pharmaceutically acceptable salt thereof, and a pharmaceutically acceptable carrier.

The present disclosure also provides a compound Formulae I′, II″, I, II, IIa, IIb, IIc, III, IIIa, IIIb, IIIc, IV, IVa, IVb, IVc, V, Va, Vb, VI, VIa, VIb, and VII, or a pharmaceutically acceptable salt thereof, for use in medicine.

The present disclosure also provides a pharmaceutical composition comprising a compound of Formulae I′, II″, I, II, IIa, IIb, IIc, III, IIIa, IIIb, IIIc, IV, IVa, IVb, IVc, V, Va, Vb, VI, VIa, VIb, and VII, or a pharmaceutically acceptable salt thereof, in association with a pharmaceutically acceptable adjuvant, diluent or carrier.

The present disclosure further provides a process for the preparation of a pharmaceutical composition of the present disclosure which comprises mixing a compound of Formulae I′, II″, I, II, ha, IIb, IIc, III, IIIa, IIIb, IIIc, IV, IVa, IVb, IVc, V, Va, Vb, VI, VIa, VIb, and VII, or a pharmaceutically acceptable salt thereof with a pharmaceutically acceptable adjuvant, diluent or carrier.

Depending on the mode of administration, the pharmaceutical composition will comprise from about 0.05 to about 99% w (percent by weight), more particularly from about 0.05 to about 80% w, still more particularly from about 0.10 to about 70% w, and even more particularly from about 0.10 to about 50% w, of active ingredient, all percentages by weight being based on total composition.

The pharmaceutical compositions may be administered topically (e.g. to the skin or to the lung and/or airways) in the form, e.g., of creams, solutions, suspensions, heptafluoroalkane (HFA) aerosols and dry powder formulations, for example, formulations in the inhaler device known as the Turbuhaler®; or systemically, e.g. by oral administration in the form of tablets, capsules, syrups, powders or granules; or by parenteral administration in the form of a sterile solution, suspension or emulsion for injection (including intravenous, subcutaneous, intramuscular, intravascular or infusion); or by rectal administration in the form of suppositories.

Dry powder formulations and pressurized HFA aerosols of the compounds of the present disclosure (including pharmaceutically acceptable salts) may be administered by oral or nasal inhalation. For inhalation, the compound is desirably finely divided. The finely divided compound preferably has a mass median diameter of less than 10 micrometres (μm), and may be suspended in a propellant mixture with the assistance of a dispersant, such as a C₈-C₂₀ fatty acid or salt thereof, (for example, oleic acid), a bile salt, a phospholipid, an alkyl saccharide, a perfluorinated or polyethoxylated surfactant, or other pharmaceutically acceptable dispersant.

The compounds of the present disclosure may also be administered by means of a dry powder inhaler. The inhaler may be a single or a multi dose inhaler, and may be a breath actuated dry powder inhaler.

One possibility is to mix the finely divided compound of the present disclosure with a carrier substance, for example, a mono-, di- or polysaccharide, a sugar alcohol, or another polyol. Suitable carriers are sugars, for example, lactose, glucose, raffinose, melezitose, lactitol, maltitol, trehalose, sucrose, mannitol; and starch. Alternatively the finely divided compound may be coated by another substance. The powder mixture may also be dispensed into hard gelatin capsules, each containing the desired dose of the active compound.

Another possibility is to process the finely divided powder into spheres which break up during the inhalation procedure. This spheronized powder may be filled into the drug reservoir of a multidose inhaler, for example, that known as the Turbuhaler® in which a dosing unit meters the desired dose which is then inhaled by the patient. With this system the active ingredient, with or without a carrier substance, is delivered to the patient.

Another possibility is to process the compound as an amorphous dispersion in a polymer matrix such as hydroxypropyl methylcellulose (HPMC) or hydroxypropyl methylcellulose acetate succinate (HPMCAS). As the name suggests, spray-dried dispersions (SDDs) are obtained by dissolving drug and polymer in an organic solvent, atomizing the resulting solution into droplets, and evaporation to dried solid particles. SDDs are usually amenable for use a variety of final oral dosage forms, including capsules and tablets.

For oral administration the compound of the present disclosure may be admixed with an adjuvant or a carrier, for example, lactose, saccharose, sorbitol, mannitol; a starch, for example, potato starch, corn starch or amylopectin; a cellulose derivative; a binder, for example, gelatin or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatin, talcum and titanium dioxide. Alternatively, the tablet may be coated with a suitable polymer dissolved in a readily volatile organic solvent.

For the preparation of soft gelatin capsules, the compound of the present disclosure may be admixed with, for example, a vegetable oil or polyethylene glycol. Hard gelatin capsules may contain granules of the compound using either the above-mentioned excipients for tablets. Also liquid or semisolid formulations of the compound of the present disclosure may be filled into hard gelatin capsules.

Liquid preparations for oral application may be in the form of syrups or suspensions, for example, solutions containing the compound of the present disclosure, the balance being sugar and a mixture of ethanol, water, glycerol and propylene glycol. Optionally such liquid preparations may contain colouring agents, flavouring agents, saccharine and/or carboxymethylcellulose as a thickening agent or other excipients known to those skilled in art.

### Methods of Treatment

The terms “treat,” “treating,” or “treatment”, as used herein, refer to any indicia of success in the amelioration of a disorder (such as injury, disease pathology, or condition), including any objective or subjective parameter such as abatement; remission; diminishing of symptoms or making the disorder more tolerable to the subject; slowing or stopping the rate of degeneration, decline, or development; slowing the progression of disorder; making the final point of degeneration less debilitating; improving a subject's physical or mental well-being; or relieving or causing regression of the disorder. The treatment of symptoms, including the amelioration of symptoms, can be based on objective or subjective parameters, which may include the results of a physical examination, a neuropsychiatric exam, and/or a psychiatric evaluation. Certain methods and uses disclosed herein may treat cancer by, for example, causing remission of cancer, slowing the rate of growth of cancer cells, slowing the rate of spread of cancer cells, reducing metastasis, or reducing the growth of metastatic tumors, reducing the size of one or more tumors, reducing the number of one or more tumors, or any combinations thereof.

The terms “administered”, “administration”, or “administering”, as used herein, refers to either directly administering a disclosed compound or pharmaceutically acceptable salt of the disclosed compound or a composition to a subject, including an animal, in need of treatment by bringing such individual in contact with, or otherwise exposing such individual to, such compound.

As used herein, the term “subject” encompasses mammals and non-mammals. Examples of mammals include, but are not limited to, any member of the class Mammalia: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the present disclosure, the mammal is a human.

A “patient” is a mammal, e.g., a human, mouse, rat, guinea pig, dog, cat, horse, cow, pig, or non-human primate, such as a monkey, chimpanzee, baboon or rhesus. “Patient” includes both humans and animals.

The term “inhibitor”, as used herein, refers to a molecule such as a compound, a drug, enzyme, or a hormone that blocks or otherwise interferes with a particular biologic activity.

The terms “effective amount” or “therapeutically effective amount”, when used in connection with a compound, refer to a sufficient amount of the compound to provide the desired biological result. That result can be reduction and/or alleviation of the signs, symptoms, or causes of a disease, or any other desired alteration of a biological system. For example, an “effective amount” for therapeutic use is the amount of the composition comprising a compound as disclosed herein required to provide a clinically significant decrease in a disease. An appropriate “effective amount” in any individual case may be determined by one of ordinary skill in the art using routine experimentation. Thus, the expression “effective amount” generally refers to the quantity for which the active substance has therapeutic effects. In the present disclosure the active substance is an inhibitor of METTL3.

The term “IC₅₀”, as used herein, refers to concentrations at which a measurable activity, phenotype or response, for example growth or proliferation of cells such as tumor cells, is inhibited by 50%. IC₅₀ values can be estimated from an appropriate dose-response curve, for example by eye or by using appropriate curve fitting or statistical software. More accurately, IC₅₀ values may be determined using non-linear regression analysis.

The term “disorder”, as used herein, refers to and is used interchangeably with the terms disease, condition, or illness, unless otherwise indicated.

In some embodiments, the present disclosure provides compounds which are suitable for use in the treatment of one or more diseases which are linked to METTL3 (e.g. the overexpression of METTL3).

In some embodiments, the compounds modulating or inhibiting METTL3 are useful for treating one or more diseases selected from neurological diseases, viral diseases, cancer, and pre-cancerous syndromes.

In some embodiments, the compounds modulating or inhibiting METTL3 are useful for treating cancer and pre-cancerous syndromes.

As used herein, cancers tumors and malignancies, refer to diseases, disorders or conditions, or to cells or tissues associated with the diseases, disorders or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumor markers, loss of tumor suppressor expression or activity and/or aberrant or abnormal cell surface marker expression. In general embodiments, cancers, tumors and malignancies may include sarcomas, lymphomas, leukemias, solid tumors, blastomas, gliomas, carcinomas, melanomas and metastatic cancers, although without limitation thereto. A more comprehensive listing of cancers tumors and malignancies may be found at the National Cancer Institutes website http://www.cancer.gov/cancertopics/types/alphalist, which is hereby incorporated by reference in its entirety.

Examples of pre-cancerous syndrome include colon polyps, cervical intraepithelial neoplasia, smoldering multiple myeloma, clonal hematopoiesis of indeterminate potential, and monoclonal gammopathy of undetermined significance.

Cancers may be in the form of solid tumors such as sarcomas, carcinomas, and lymphomas or in the form of liquid tumors, e.g. leukemia. Certain blood cancers include leukemia, lymphoma and multiple myeloma. Certain leukemia include acute lymphocytic leukemia (ALL), Acute myeloid leukemia (AML), chronic lymphocytic leukemia (CLL), chronic myeloid leukemia (CML), and myelodysplastic syndromes (MDS). A certain cancer is AML. Certain brain cancers include glioblastoma, oligodendroglioma, meningioma. A certain cancer is glioblastoma. Certain lung cancers include small-cell lung cancer (SCLC) and non-small cell lung cancer (NSCLC). A certain cancer is NSCLC. A certain cancer is hepatocellular carcinoma (HCC).

In some embodiments, the compounds are suitable for use in the treatment of cancer. In some embodiments, the compounds are suitable for us in the treatment of solid or liquid tumors. In some embodiments, the compounds are suitable for use in the treatment of liquid tumors. In some embodiments, the compounds are suitable for use in the treatment of blood cancer, pancreatic cancer, gastric cancer, colorectal cancer, ovarian cancer, endometrial cancer, bladder cancer, breast cancer, brain cancer, melanoma, lung cancer, or liver cancer. In some embodiments, the compounds are suitable for use in the treatment of blood cancer or acute myeloid leukemia (AML).

In some embodiments, the compounds are suitable for use in the treatment of neurological diseases.

Neurological diseases include diseases of the central and peripheral nervous system. These disorders include epilepsy, Alzheimer disease and other dementias, cerebrovascular diseases including stroke, migraine and other headache disorders, multiple sclerosis, motor neuron diseases, Parkinson's disease, neuroinfections, brain tumours, traumatic disorders of the nervous system due to head trauma, or neurological disorders as a result of malnutrition. Of particular relevance is amyotrophic lateral sclerosis (ALS).

In some embodiments, the compounds are suitable for use in the treatment of viral diseases.

Viral diseases may be caused by DNA viruses or RNA viruses. Viral diseases may be caused by certain virus families such as Adenoviridae, Herpesviridae, Papillomaviridae, Polyomaviridae, Poxviridae, Hepadnaviridae, Parvoviridae, Astroviridae, Caliciviridae, Picor-naviridae, Coronaviridae, Flaviviridae, Togaviridae, Hepeviridae, Retroviridae, Orthomyxoviridae, Arenaviridae, Bunyaviridae, Filoviridae, Paramyxoviridae, Rhabdoviridae, or Reoviridae.

For the above-mentioned therapeutic uses the dosage administered will, of course, vary with the compound employed, the mode of administration, the treatment desired and the disorder indicated. For example, the daily dosage of the compound of the present disclosure, if inhaled, may be in the range from about 0.05 micrograms per kilogram body weight (μg/kg) to about 100 micrograms per kilogram body weight (μg/kg). Alternatively, if the compound is administered orally, then the daily dosage of the compound of the present disclosure may be in the range from about 0.01 micrograms per kilogram body weight (μg/kg) to about 100 milligrams per kilogram body weight (mg/kg).

The compounds of Formulae I′, II″, I, II, IIa, IIb, IIc, III, IIIa, IIIb, IIIc, IV, IVa, IVb, IVc, V, Va, Vb, VI, VIa, VIb, and VII and pharmaceutically acceptable salts thereof have activity as pharmaceuticals, as discussed herein.

The present disclosure provides a method of treatment of a disease, disorder or condition including the step of administering an effective amount of a compound of Formulae I′, II″, I, II, IIa, IIb, IIc, III, IIIa, IIIb, IIIc, IV, IVa, IVb, IVc, V, Va, Vb, VI, VIa, VIb, and VII or a pharmaceutically acceptable salt thereof to thereby treat the disease, disorder or condition in a subject in need thereof.

The present disclosure provides a compound of Formulae I′, II″, I, II, IIa, IIb, IIc, III, IIIa, IIIb, IIIc, IV, IVa, IVb, IVc, V, Va, Vb, VI, VIa, VIb, and VII, or pharmaceutically acceptable salts thereof, or a pharmaceutical composition thereof for use in the treatment of a disease, disorder or condition in a subject in need thereof.

The present disclosure provides a compound of Formulae I′, II″, I, II, IIa, IIb, IIc, III, IIIa, IIIb, IIIc, IV, IVa, IVb, IVc, V, Va, Vb, VI, VIa, VIb, and VII, or pharmaceutically acceptable salts thereof, or a pharmaceutical composition thereof for the treatment of a disease, disorder or condition in a subject in need thereof.

The present disclosure provides a compound of Formulae I′, II″, I, II, IIa, IIb, IIc, III, IIIa, IIIb, IIIc, IV, IVa, IVb, IVc, V, Va, Vb, VI, VIa, VIb, and VII, or pharmaceutically acceptable salts thereof, or a pharmaceutical composition thereof, for use in the treatment of a disorder that is responsive to modulation or inhibition of METTL3.

The present disclosure provides a compound of Formulae I′, II″, I, II, IIa, IIb, IIc, III, IIIa, IIIb, IIIc, IV, IVa, IVb, IVc, V, Va, Vb, VI, VIa, VIb, and VII, or pharmaceutically acceptable salts thereof, or a pharmaceutical composition thereof, in the treatment of a disorder that is responsive to modulation or inhibition of METTL3.

The present disclosure provides a compound of Formulae I′, II″, I, II, IIa, IIb, IIc, III, IIIa, IIIb, IIIc, IV, IVa, IVb, IVc, V, Va, Vb, VI, VIa, VIb, and VII, or pharmaceutically acceptable salts thereof, or a pharmaceutical composition thereof, for use in the manufacture of a medicament for the treatment of a disorder that is responsive to modulation or inhibition of METTL3.

The present disclosure also provides a method of treating a disorder in a subject in need thereof, wherein the disorder is mediated by METTL3, comprising administering to the subject an effective amount of a compound of Formulae I′, II″, I, II, IIa, IIb, IIc, III, IIIa, IIIb, IIIc, IV, IVa, IVb, IVc, V, Va, Vb, VI, VIa, VIb, and VII, or pharmaceutically acceptable salts thereof, or a pharmaceutical composition thereof.

In some embodiments, the disease, disorder, or condition is selected from the group consisting of neurological diseases, viral diseases, cancer, and pre-cancerous syndromes.

In some embodiments, the disorder is cancer and the cancer is selected from the group consisting of blood cancer, pancreatic cancer, gastric cancer, colorectal cancer, ovarian cancer, endometrial cancer, bladder cancer, breast cancer, brain cancer, melanoma, lung cancer, and liver cancer.

In some embodiments, the disorder is leukemia.

In some embodiments, the disorder is acute myeloid leukemia (AML).

### Combination Therapy

The compounds of Formulae I′, II″, I, II, IIa, IIb, IIc, III, IIIa, IIIb, IIIc, IV, IVa, IVb, IVc, V, Va, Vb, VI, VIa, VIb, and VII and pharmaceutically acceptable salts thereof may also be administered in conjunction with other compounds used for the treatment of the above conditions.

Some embodiments further relate to combination therapies wherein a compound of the present disclosure or a pharmaceutical composition or formulation comprising a compound of the present disclosure is administered concurrently or sequentially or as a combined preparation with another therapeutic agent or agents, for the treatment of one or more of the conditions listed.

### Example Embodiments

### Embodiment I-1. A Compound of Formula (I′)

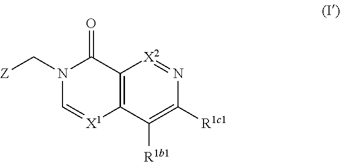

or a pharmaceutically acceptable salt thereof, wherein:
X¹ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X² is CH or N;
R^{1b1} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b1} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b1} is unsubstituted or substituted with one or more substituents selected from the group consisting of aryl, heterocyclyl, and cycloalkyl;
R^{1c1} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl; wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
Z is

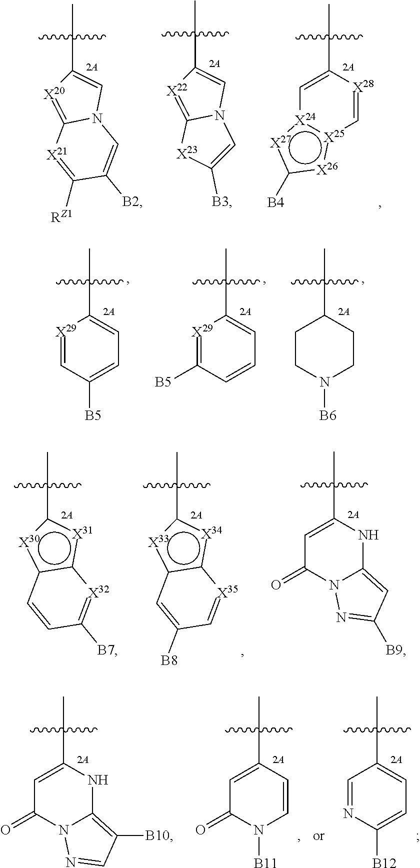

X²⁰ is CR^{20a} or N; wherein R^{20a} is H, halo, CN, aryl, or heteroaryl;
X²¹ is CH or N;
X²² is CR^{22a} or N; wherein R^{22a} is H, halo, CN, aryl, or heteroaryl;
X²³ is CH₂, S, O, NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
X²⁹ is CH or N;
X³⁰ and X³¹ are independently CH, O, S, N, or NH;
X³² is CH or N;
X³³ and X³⁴ are independently CH, O, S, N, or NH;
X³⁵ is CH or N;
R^{Z1} is H, halo, —CN, or —S(O)₂F;
B2, B3, B4, B7, B8, B9, and B10 are independently selected from the group consisting of H, C₁₋₆alkyl, CN, —S(O)₂F, —CH₂—NR^{2a}R^{2b},

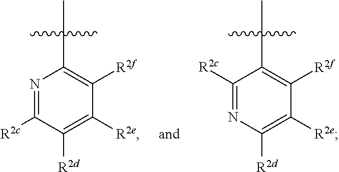

R^{2a} is H or C₁₋₆alkyl;
R^{2b} is selected from the group consisting of H, C₁₋₆alkyl, aryl, heteroaryl, heterocyclyl, —C(NH)-aryl, —S(O)(NH)R^{2g}, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, —S(O)₂F, —S(O)NH)R^{2g}, alkoxy, aryl, haloaryl, cycloalkyl, halocycloalkyl, hydroxycycloalkyl, and heterocyclyl, wherein the cycloalkyl is unsubstituted or substituted with one or more substituents selected from the group consisting of C₁₋₆alkyl, —(CH₂)ₙF, —(CH₂)ₙBr, or —(CH₂)ₙCN and wherein the heterocyclyl is unsubstituted or substituted with C₁₋₆alkyl; or
R^{2a} and R^{2b} combine to form a 5-7-membered heterocyclyl, wherein the heterocyclyl is unsubstituted or substituted with one or more substituents selected from the group consisting of C₁₋₆alkyl and cycloalkyl;
R^{2c}, R^{2d}, R^{2e}, and R^{2f} are each independently H, C₁₋₆alkyl, heterocyclyl, or —NHR²8;
R^{2g} is selected from the group consisting of H, C₁₋₆alkyl, and cycloalkyl, wherein the C₁₋₆alkyl is unsubstituted or substituted with cycloalkyl;
each R^{2h} is independently H, C₁₋₆alkyl, —(CH₂)ₙCl, or —(CH₂)·OCH₃;
n is 1, 2, 3, 4, 5, or 6;
B5, B6, B11, and B12 are independently

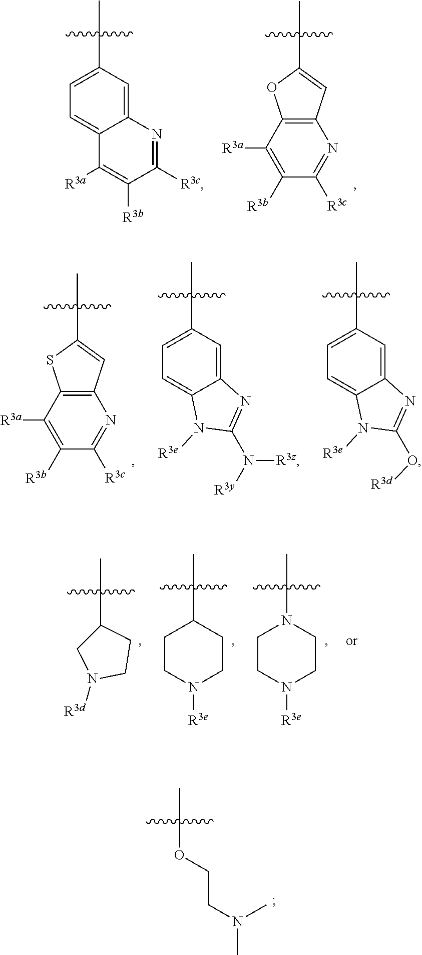

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, —OR^{3x′}, and —CO₂R^{x};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

### Embodiment I-2. A Compound of Formula (I)

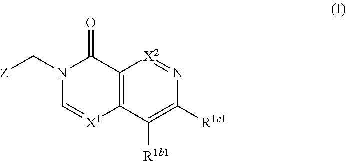

or a pharmaceutically acceptable salt thereof, wherein:
X¹ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X² is CH or N;
R^{1b1} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b1} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b1} is unsubstituted or substituted with one or more substituents selected from the group consisting of aryl, heterocyclyl, and cycloalkyl;
R^{1c1} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
Z is

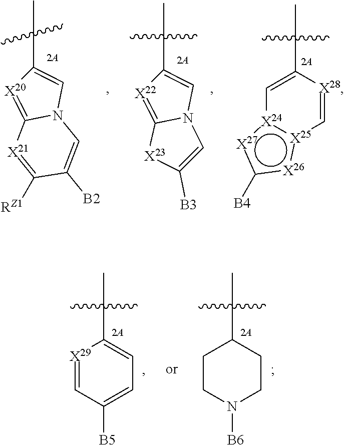

X²⁰ is CR^{20a} or N; wherein R^{20a} is H, halo, CN, aryl, or heteroaryl;
X²¹ is CH or N;
X²² is CR^{22a} or N; wherein R^{22a} is H, halo, CN, aryl, or heteroaryl;
X²³ is CH₂, S, O, NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
X²⁹ is CH or N;
B2, B3, and B4 are independently selected from the group consisting of H, C₁₋₆alkyl, CN, —CH₂—NR^{2a}R^{2b},

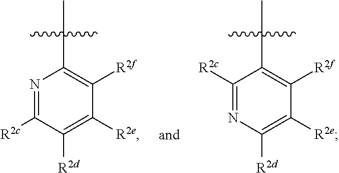

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;
B5 and B6 are independently

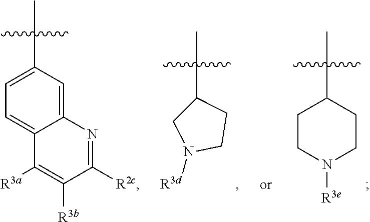

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

### Embodiment I-3. A Compound of Formula (II), (III), (IV), (V), or (VI)

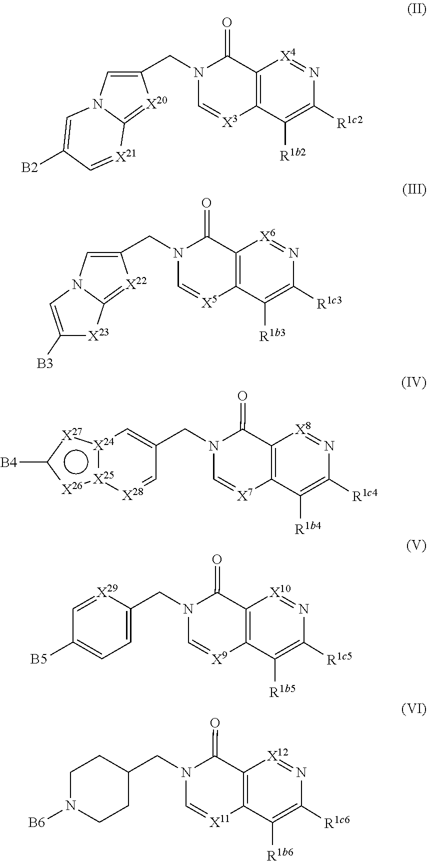

or a pharmaceutically acceptable salt thereof, wherein:
X³, X⁵, X⁷, X⁹, and X¹¹ are independently CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X⁴, X⁶, X⁸, X¹⁰, and X¹² are independently CH or N;
R^{1b2}, R^{1b3}, R^{1b4}, R^{1b5}, and R^{1b6} are independently selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b2}, R^{1b3}, R^{1b4}, R^{1b5}, and R^{1b6} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b2}, R^{1b3}, R^{1b4}, R^{1b5}, and R^{1b6} is unsubstituted or substituted with one or more substituents selected from the group consisting of aryl, heterocyclyl, and cycloalkyl;
R^{1c2}, R^{1c3}, and R^{1c4} are independently selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl; wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁰ is CR^{20a} or N; wherein R^{20a} is H, halo, CN, aryl, or heteroaryl;
X²¹ is CH or N;
X²² is CR^{22a} or N; wherein R^{22a} is H, halo, aryl, or heteroaryl;
X²³ is CH, S, O, or NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
B2, B3, and B4 are independently selected from the group consisting of H, C₁₋₆alkyl, CN, —CH₂—NR^{2a}R^{2b},

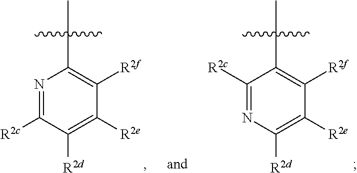

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;
X²⁹ is CH or N;
B5 and B6 are independently

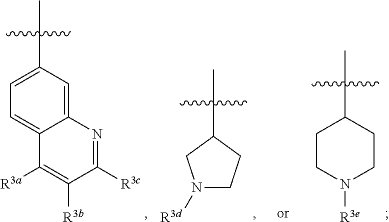

R³, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted cycloalkyl; and
R^{3e} is selected from the group consisting of H and C₁₋₆alkyl; wherein C₁₋₆alkyl is unsubstituted or substituted cycloalkyl.

### Embodiment I-4. A Compound of Formula (II)

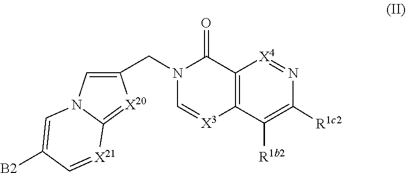

or a pharmaceutically acceptable salt thereof, wherein:
X³ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X⁴ is CH or N;
R^{1b2} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b2} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b2} is unsubstituted or substituted with one or more substituents selected from the group consisting of aryl, heterocyclyl, and cycloalkyl;
R^{1c2} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl; wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁰ is CR^{20a} or N; wherein R^{20a} is H, halo, CN, aryl, or heteroaryl;
X²¹ is CH or N;
B2 is selected from the group consisting of H, C₁₋₆alkyl, CN, —CH₂—NR^{2a}R^{2b},

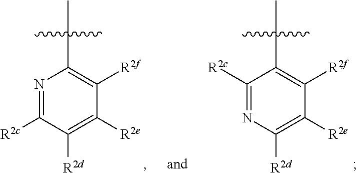

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

Embodiment I-5. The compound of embodiment I-4, or a pharmaceutically acceptable salt thereof, wherein the compound is of Formula (IIa):

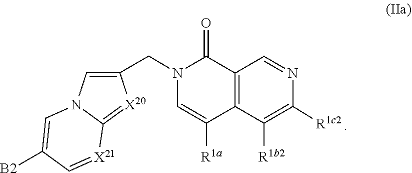

Embodiment I-6. The compound of embodiment I-4 or I-5, or a pharmaceutically acceptable salt thereof, wherein the compound is of Formula (IIb):

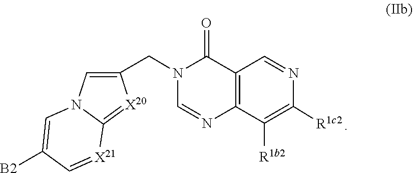

Embodiment I-7. The compound of any one of embodiments I-4, I-5, and I-6, or a pharmaceutically acceptable salt thereof, wherein the compound is of Formula (IIc):

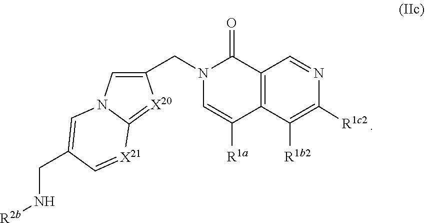

Embodiment I-8. A compound of Formula (III):

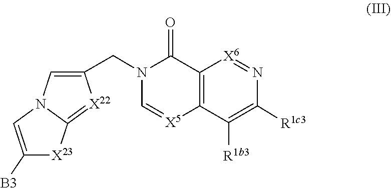

or a pharmaceutically acceptable salt thereof, wherein:
X⁵ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X⁶ is CH or N;
R^{1b3} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b3} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b3} is unsubstituted or substituted with one or more substituents selected from the group consisting of aryl, heterocyclyl, and cycloalkyl;
R^{1c3} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl; wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²² is CR^{22a} or N; wherein R^{22a} is H, halo, aryl, or heteroaryl;
X²³ is CH, S, O, or NH;
B3 is selected from the group consisting of H, C₁₋₆alkyl, CN, —CH₂—NR^{2a}R^{2b},

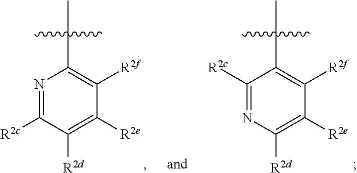

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

Embodiment I-9. The compound of embodiment I-8, or a pharmaceutically acceptable salt thereof, wherein the compound is of Formula (IIIa):

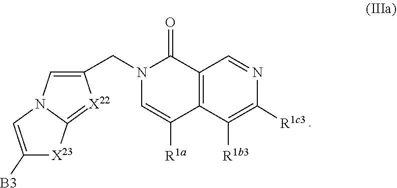

Embodiment I-10. The compound of embodiment I-8 or I-9, or a pharmaceutically acceptable salt thereof, wherein the compound is of Formula (IIIb):

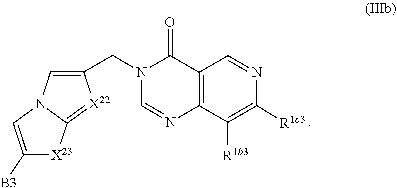

Embodiment I-11. The compound of any one of embodiments I-8, I-9, and I-10, or a pharmaceutically acceptable salt thereof, wherein the compound is of Formula (IIIc):

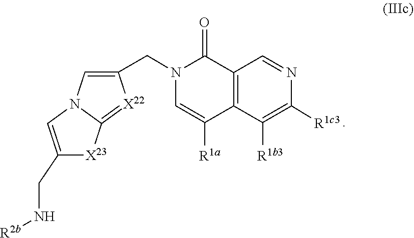

Embodiment I-12. A compound of Formula(IV):

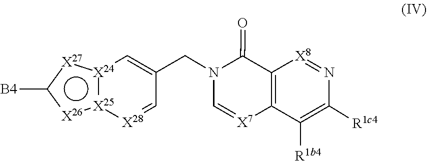

or a pharmaceutically acceptable salt thereof, wherein:
X⁷ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X⁸ is CH or N;
R^{1b4} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b4} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b4} is unsubstituted or substituted with one or more substituents selected from the group consisting of aryl, heterocyclyl, and cycloalkyl;
R^{1c4} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
B4 is selected from the group consisting of H, C₁₋₆alkyl, CN, —CH₂—NR^{2a}R^{2b},

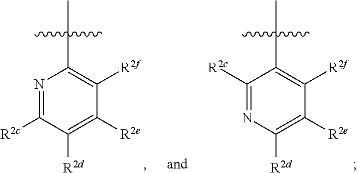

R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

Embodiment I-13. The compound of embodiment I-12, or a pharmaceutically acceptable salt thereof, wherein the compound is of Formula IVa):

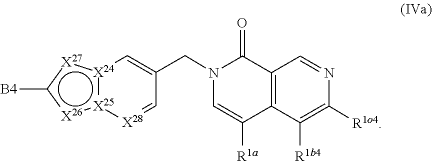

Embodiment I-14. The compound of embodiment I-12 or I-13, or a pharmaceutically acceptable salt thereof, wherein the compound is of Formula (IVb):

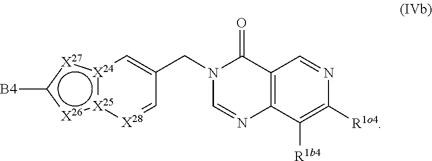

Embodiment I-15. The compound of any one of embodiments I-12, I-13 or I-14, or a pharmaceutically acceptable salt thereof, wherein the compound is of Formula (IVc):

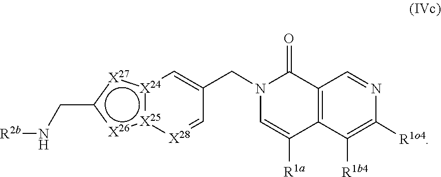

Embodiment I-16. A compound of Formula(V):

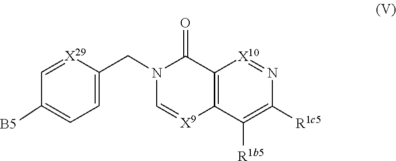

or a pharmaceutically acceptable salt thereof, wherein:
X⁹ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X¹⁰ is CH or N;
R^{1b5} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b5} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b5} is unsubstituted or substituted with one or more substituents selected from the group consisting of aryl, heterocyclyl, and cycloalkyl;
R^{1e} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁹ is CH or N;
B5 is

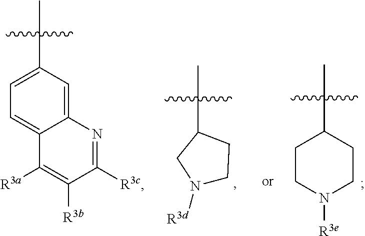

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

Embodiment I-17. The compound of embodiment I-16, or a pharmaceutically acceptable salt thereof, wherein the compound is of Formula (Va):

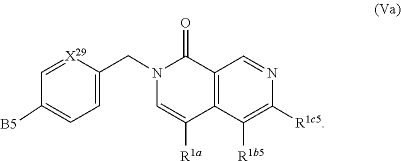

Embodiment I-18. The compound of embodiment I-16 or I-17, or a pharmaceutically acceptable salt thereof, wherein the compound is of Formula (Vb):

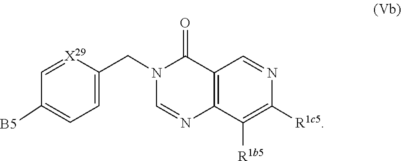

Embodiment I-19. A compound of Formula(VI):

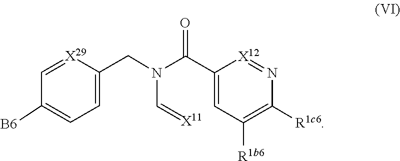

or a pharmaceutically acceptable salt thereof, wherein:
X¹¹ is CR^{1a} or N;
R^{1a} is selected from the group consisting of H, C₁₋₆alkyl, halo, cycloalkyl, and heteroaryl; wherein the C₁₋₆alkyl of R^{1a} is unsubstituted or substituted with aryl;
X¹² is CH or N;
R^{1b6} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b6} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b6} is unsubstituted or substituted with one or more substituents selected from the group consisting of aryl, heterocyclyl, and cycloalkyl;
R^{1c6} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
B6 is

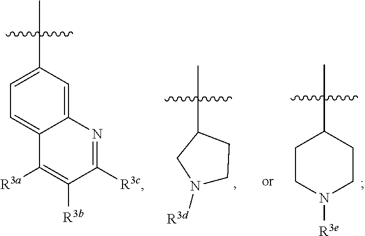

R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

Embodiment I-20. The compound of embodiment I-19, or a pharmaceutically acceptable salt thereof, wherein the compound is of Formula (VIa):

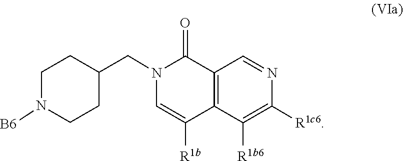

Embodiment I-21. The compound of embodiment I-19 or I-20, or a pharmaceutically acceptable salt thereof, wherein the compound is of Formula (VIb):

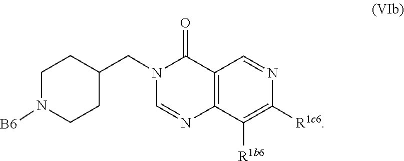

Embodiment I-22. The compound of any one of embodiments I-1 to I-4, I-8, I-12, I-16, and I-19, or a pharmaceutically acceptable salt thereof, wherein X¹, X³, X⁵, X⁷, X⁹, or X¹¹ is CR^{1a}.

Embodiment I-23. The compound of any one of embodiments I-1 to I-4, I-6, I-12, I-16, I-19, and I-22, or a pharmaceutically acceptable salt thereof, wherein R^{1a} is H.

Embodiment I-24. The compound of any one of embodiments I-1 to I-4, I-6, I-12, I-16, I-19, and I-22, or a pharmaceutically acceptable salt thereof, wherein R^{1a} is C₁₋₆alkyl, wherein the C₁₋₆alkyl is unsubstituted or substituted with aryl.

Embodiment I-25. The compound of any one of embodiments I-1 to I-4, I-6, I-12, I-16, I-19, and I-22, or a pharmaceutically acceptable salt thereof, wherein R^{1a} is cycloalkyl or heteroaryl.

Embodiment I-26. The compound of any one of embodiments I-1 to I-4, I-6, I-12, I-16, I-19, and I-22, or a pharmaceutically acceptable salt thereof, wherein R^{1a} is halo.

Embodiment I-27. The compound of any one of embodiments I-1 to I-4, I-8, I-12, I-16, and I-19, or a pharmaceutically acceptable salt thereof, wherein X¹, X³, X⁵, X⁷, X⁹, or X¹¹ is N.

Embodiment I-28. The compound of any one of embodiments I-1 to I-4, I-6, I-12, I-16, I-19, and I-22 to I-27, or a pharmaceutically acceptable salt thereof, wherein X², X⁴, X⁶, X⁸, X¹⁰, or X¹² is CH.

Embodiment I-29. The compound of any one of embodiments I-1 to I-4, I-6, I-12, I-16, I-19, and I-22 to I-27, or a pharmaceutically acceptable salt thereof, wherein X², X⁴, X⁶, X⁸, X¹⁰, or X¹² is N.

Embodiment I-30. The compound of any one of embodiments I-1 to I-29, or a pharmaceutically acceptable salt thereof, wherein R^{1c1}, R^{1c2}, R^{1c3}, R^{1c4}, R^{1c5}, or R^{1c6} is H.

Embodiment I-31. The compound of any one of embodiments I-1 to I-29, or a pharmaceutically acceptable salt thereof, wherein R^{1c1}, R^{1c2}, R^{1c3}, R^{1c4}, R^{1c5}, or R^{1c6} is C₁₋₆alkyl.

Embodiment I-32. The compound of any one of embodiments I-1 to I-29, or a pharmaceutically acceptable salt thereof, wherein R^{1c1}, R^{1c2}, R^{1c3}, R^{1c4}, R^{1c5}, or R^{1c6} is halo, —CN, —N(R^{1d})₂, or —OR^{1d}.

Embodiment I-33. The compound of any one of embodiments I-1 to I-29 and I-32, or a pharmaceutically acceptable salt thereof, wherein R^{1d} is H or C₁₋₆alkyl.

Embodiment I-34. The compound of any one of embodiments I-1 to I-29 and I-32, or a pharmaceutically acceptable salt thereof, wherein R^{1d} is —(CH₂)ₘCN or —(CH₂)ₘNH₂.

Embodiment I-35. The compound of any one of embodiments I-1 to I-29 and I-32, or a pharmaceutically acceptable salt thereof, wherein R^{1d} is cycloalkyl or heterocyclyl.

Embodiment I-36. The compound of any one of embodiments I-1 to I-35, or a pharmaceutically acceptable salt thereof, wherein R^{1b1}, R^{1b2}, R^{1b3}, R^{1b4}, R^{1b5}, or R^{1b6} is aryl, wherein the aryl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}.

Embodiment I-37. The compound of any one of embodiments I-1 to I-35, or a pharmaceutically acceptable salt thereof, wherein R^{1b1}, R^{1b2}, R^{1b3}, R^{1b4}, R^{1b5}, or R^{1b6} is heteroaryl or heterocyclyl, wherein the heteroaryl and heterocyclyl of R^{1b} is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}.

Embodiment I-38. The compound of any one of embodiments I-1 to I-35, or a pharmaceutically acceptable salt thereof, wherein R^{1b1}, R^{1b2}, R^{1b3}, R^{1b4}, R^{1b5}, or R^{1b6} is cycloalkyl or cycloalkenyl, wherein the cycloalkyl and cycloalkenyl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}.

Embodiment I-39. The compound of any one of embodiments I-36 to I-38, or a pharmaceutically acceptable salt thereof, wherein the aryl, cycloalkyl, cycloalkenyl, heteroaryl, and heterocyclyl is unsubstituted.

Embodiment I-40. The compound of any one of embodiments I-1 to I-35, or a pharmaceutically acceptable salt thereof, wherein R^{1b1}, R^{1b2}, R^{1b3}, R^{1b4}, R^{1b5}, or R^{1b6} is C₁₋₆alkyl or alkynyl, wherein the C₁₋₆alkyl and alkynyl is unsubstituted or substituted with one or more substituents selected from the group consisting of aryl, heterocyclyl, and cycloalkyl.

Embodiment I-41. The compound of any one of embodiments I-1 to I-35, or a pharmaceutically acceptable salt thereof, wherein R^{1b1}, R^{1b2}, R^{1b3}, R^{1b4}, R^{1b5}, or R^{1b6} is H.

Embodiment I-42. The compound of any one of embodiments I-1 to I-35, or a pharmaceutically acceptable salt thereof, wherein R^{1b1}, R^{1b2}, R^{1b3}, R^{1b4}, R^{1b5}, or R^{1b6} is halo.

Embodiment I-43. The compound of any one of embodiments I-1 to I-35, or a pharmaceutically acceptable salt thereof, wherein R^{1b1}, R^{1b2}, R^{1b3}, R^{1b4}, R^{1b5}, or R^{1b6} is —OR^{1e} or —N(R^{1e}).

Embodiment I-44. The compound of any one of embodiments I-1 to I-7 and I-22 to I-43, or a pharmaceutically acceptable salt thereof, wherein X²⁰ is CR^{20a}.

Embodiment I-45. The compound of any one of embodiments I-1 to I-7 and I-22 to I-44, or a pharmaceutically acceptable salt thereof, wherein R^{20a} is H.

Embodiment I-46. The compound of any one of embodiments I-1 to I-7 and I-22 to I-44, or a pharmaceutically acceptable salt thereof, wherein R^{20a} is halo, CN, aryl, or heteroaryl.

Embodiment I-47. The compound of any one of embodiments I-1 to I-7 and I-22 to I-43, or a pharmaceutically acceptable salt thereof, wherein X²⁰ is N.

Embodiment I-48. The compound of any one of embodiments I-1 to I-7 and I-22 to I-47, or a pharmaceutically acceptable salt thereof, wherein X²¹ is CH.

Embodiment I-49. The compound of any one of embodiments I-1 to I-7 and I-22 to I-47, or a pharmaceutically acceptable salt thereof, wherein X²¹ is N.

Embodiment I-50. The compound of any one of embodiments I-1 to I-3, I-8 to I-11, and I-22 to I-43, or a pharmaceutically acceptable salt thereof, wherein X²² is CR^{22a}.

Embodiment I-51. The compound of any one of embodiments I-1 to I-3, I-8 to I-11, I-22 to I-43, and I-50, or a pharmaceutically acceptable salt thereof, wherein R^{22a} is H.

Embodiment I-52. The compound of any one of embodiments I-1 to I-3, I-8 to I-11, I-22 to I-43, and I-50, or a pharmaceutically acceptable salt thereof, wherein R^{22a} is halo or aryl.

Embodiment I-53. The compound of any one of embodiments I-1 to I-3, I-8 to I-11, and I-22 to I-43, or a pharmaceutically acceptable salt thereof, wherein X²² is N.

Embodiment I-54. The compound of any one of embodiments I-1 to I-3, I-8 to I-11, I-22 to I-43, and I-50 to I-53, or a pharmaceutically acceptable salt thereof, wherein X²³ is CH₂.

Embodiment I-55. The compound of any one of embodiments I-1 to I-3, I-8 to I-11, I-22 to I-43, and I-50 to I-53, or a pharmaceutically acceptable salt thereof, wherein X²³ is NH.

Embodiment I-56. The compound of any one of embodiments I-1 to I-3, I-8 to I-11, I-22 to I-43, and I-50 to I-53, or a pharmaceutically acceptable salt thereof, wherein X²³ is S or O.

Embodiment I-57. The compound of any one of embodiments I-1 to I-3, I-12 to I-15, and I-22 to I-43, or a pharmaceutically acceptable salt thereof, wherein X²⁴ and X²⁵ are C.

Embodiment I-58. The compound of any one of embodiments I-1 to I-3, I-12 to I-15, and I-22 to I-43, or a pharmaceutically acceptable salt thereof, wherein X²⁴ is N and X²⁵ is C.

Embodiment I-59. The compound of any one of embodiments I-1 to I-3, I-12 to I-15, I-22 to I-43, and I-57 to I-58, or a pharmaceutically acceptable salt thereof, wherein X²⁶ and X²⁷ are independently N or NR²⁶; wherein R²⁶ is H or C₁₋₆alkyl.

Embodiment I-60. The compound of any one of embodiments I-1 to I-3, I-12 to I-15, I-22 to I-43, and I-57 to I-58, or a pharmaceutically acceptable salt thereof, wherein X²⁶ and X²⁷ are CH.

Embodiment I-61. The compound of any one of embodiments I-1 to I-3, I-12 to I-15, I-22 to I-43, and I-57 to I-60, or a pharmaceutically acceptable salt thereof, wherein X²⁸ is CH.

Embodiment I-62. The compound of any one of embodiments I-1 to I-3, I-12 to I-15, I-22 to I-43, and I-57 to I-60, or a pharmaceutically acceptable salt thereof, wherein X²⁸ is N.

Embodiment I-63. The compound of any one of embodiments I-1 to I-6, I-8 to I-10, I-12 to I-14, and I-22 to I-62, or a pharmaceutically acceptable salt thereof, wherein B2, B3, or B4 is —CH₂—NR^{2a}R^{2b}.

Embodiment I-64. The compound of any one of embodiments I-1 to I-15 and I-22 to I-63, or a pharmaceutically acceptable salt thereof, wherein R^{2b} is C₁₋₆alkyl, wherein the C₁₋₆alkyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl.

Embodiment I-65. The compound of any one of embodiments I-1 to I-15 and I-22 to I-63, or a pharmaceutically acceptable salt thereof, wherein R^{2b} is cycloalkyl; wherein the cycloalkyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl.

Embodiment I-66. The compound of any one of embodiments I-1 to I-15 and I-22 to I-63, or a pharmaceutically acceptable salt thereof, wherein R^{2b} is selected from the group consisting of heteroaryl and heterocyclyl; wherein the heteroaryl and heterocyclyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl.

Embodiment I-67. The compound of any one of embodiments I-1 to I-6, I-8 to I-10, I-12 to I-14, and I-22 to I-66, or a pharmaceutically acceptable salt thereof, wherein R^{2a} is H.

Embodiment I-68. The compound of any one of embodiments I-1 to I-6, I-8 to I-10, I-12 to I-14, and I-22 to I-63, or a pharmaceutically acceptable salt thereof, wherein R^{2a} and R^{2b} combine to form a 5-7-membered heterocyclyl, wherein the heterocyclyl is substituted with C₁₋₆alkyl.

Embodiment I-69. The compound of any one of embodiments I-1 to I-6, I-8 to I-10, 1-12 to I-14, and I-22 to I-63, or a pharmaceutically acceptable salt thereof, wherein R^{2a} and R^{2b} combine to form a 5-7-membered heterocyclyl, wherein the heterocyclyl is unsubstituted.

Embodiment I-70. The compound of any one of embodiments I-1 to I-6, I-8 to I-10, I-12 to I-14, and I-22 to I-62, or a pharmaceutically acceptable salt thereof, wherein B2, B3, or B4 is

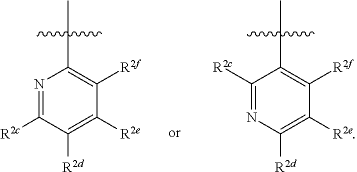

Embodiment I-71. The compound of any one of embodiments I-1 to I-6, I-8 to I-10, I-12 to I-14, I-22 to I-62, and I-70, or a pharmaceutically acceptable salt thereof, wherein one of R^{2c}, R^{2d}, R^{2e}, and R^{2f} is —NHR^{2g} and the others are H.

Embodiment I-72. The compound of any one of embodiments I-1 to I-6, I-8 to I-10, I-12 to I-14, I-22 to I-62, and I-70 to I-71, or a pharmaceutically acceptable salt thereof, wherein R^{2g} is C₁₋₆alkyl, wherein the C₁₋₆alkyl is unsubstituted or substituted with cycloalkyl.

Embodiment I-73. The compound of any one of embodiments I-1 to I-6, I-8 to I-10, I-12 to I-14, I-22 to I-62, and I-70 to I-71, or a pharmaceutically acceptable salt thereof, wherein R^{2g} is H.

Embodiment I-74. The compound of any one of embodiments I-1 to I-6, I-8 to I-10, I-12 to I-14, I-22 to I-62, and I-70, or a pharmaceutically acceptable salt thereof, wherein one of R^{2c}, R^{2d}, R^{2e}, and R^{2f} is C₁₋₆alkyl or heterocyclyl and the others are H.

Embodiment I-75. The compound of any one of embodiments I-1 to I-6, I-8 to I-10, I-12 to I-14, and I-22 to I-62, or a pharmaceutically acceptable salt thereof, wherein B2, B3, or B4 is C₁₋₆alkyl.

Embodiment I-76. The compound of any one of embodiments I-1 to I-6, I-8 to I-10, I-12 to I-14, and I-22 to I-62, or a pharmaceutically acceptable salt thereof, wherein B2, B3, or B4 is H or CN.

Embodiment I-77. The compound of any one of embodiments I-1 to I-3, I-16 to I-18, and I-22 to I-43, or a pharmaceutically acceptable salt thereof, wherein X²⁹ is CH.

Embodiment I-78. The compound of any one of embodiments I-1 to I-3, I-16 to I-18, and I-22 to I-43, or a pharmaceutically acceptable salt thereof, wherein X²⁹ is N.

Embodiment I-79. The compound of any one of embodiments I-1 to I-3, I-16 to I-43, and I-77 to I-78, or a pharmaceutically acceptable salt thereof, wherein B5 or B6 is

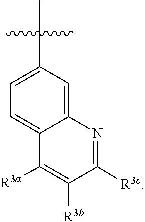

Embodiment I-80. The compound of any one of embodiments I-1 to I-3, I-16 to I-43, and I-77 to I-79, or a pharmaceutically acceptable salt thereof, wherein one or two of R^{3a}, R^{3b}, and R^{3c} is —OR^{3x} and the others of R^{3a}, R^{3b}, and R^{3c} are H.

Embodiment I-81. The compound of any one of embodiments I-1 to I-3, I-16 to I-43, and I-77 to I-80, or a pharmaceutically acceptable salt thereof, wherein R^{3x} is C₁₋₆alkyl.

Embodiment I-82. The compound of any one of embodiments I-1 to I-3, I-16 to I-43, and I-77 to I-79, or a pharmaceutically acceptable salt thereof, wherein one or two of R^{3a}, R^{3b}, and R^{3c} is —NR³YR^{3z} and the others of R^{3a}, R^{3b}, and R^{3c} are H.

Embodiment I-83. The compound of any one of embodiments I-1 to I-3, I-16 to I-43, I-77 to I-79, and I-82, or a pharmaceutically acceptable salt thereof, wherein one of R^{3y} and R^{3z} is C₁₋₆alkyl; wherein C₁₋₆alkyl is unsubstituted or substituted with one or more substituents selected from the group consisting of cycloalkyl and —OCH₃.

Embodiment I-84. The compound of any one of embodiments I-1 to I-3, I-16 to I-43, and I-77 to I-79, or a pharmaceutically acceptable salt thereof, wherein one or two of R^{3a}, R^{3b}, and R^{3c} is heterocyclyl wherein the heterocyclyl is unsubstituted or substituted with one or more substituents selected from the group consisting of C₁₋₆alkyl, halogen, and —OH, and the others of R^{3a}, R^{3b}, and R^{c} are H.

Embodiment I-85. The compound of any one of embodiments I-1 to I-3, I-16 to I-43, and I-77 to I-78, or a pharmaceutically acceptable salt thereof, wherein B5 or B6 is

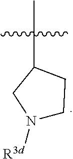

Embodiment I-86. The compound of any one of embodiments I-1 to I-3, I-16 to I-43, I-77 to I-78, and I-85, or a pharmaceutically acceptable salt thereof, wherein R^{3d} is C₁₋₆alkyl; wherein C₁₋₆alkyl is unsubstituted or substituted with cycloalkyl.

Embodiment I-87. The compound of any one of embodiments I-1 to I-3, I-16 to I-43, and I-77 to I-78, or a pharmaceutically acceptable salt thereof, wherein B5 or B6 is

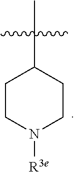

Embodiment I-88. The compound of any one of embodiments I-1 to I-3, I-16 to I-43, I-77 to I-78, and I-87, or a pharmaceutically acceptable salt thereof, wherein R^{3e} is C₁₋₆alkyl; wherein C₁₋₆alkyl is unsubstituted or substituted with cycloalkyl.

Embodiment I-89. A compound, or a pharmaceutically acceptable salt thereof, selected from the group consisting of

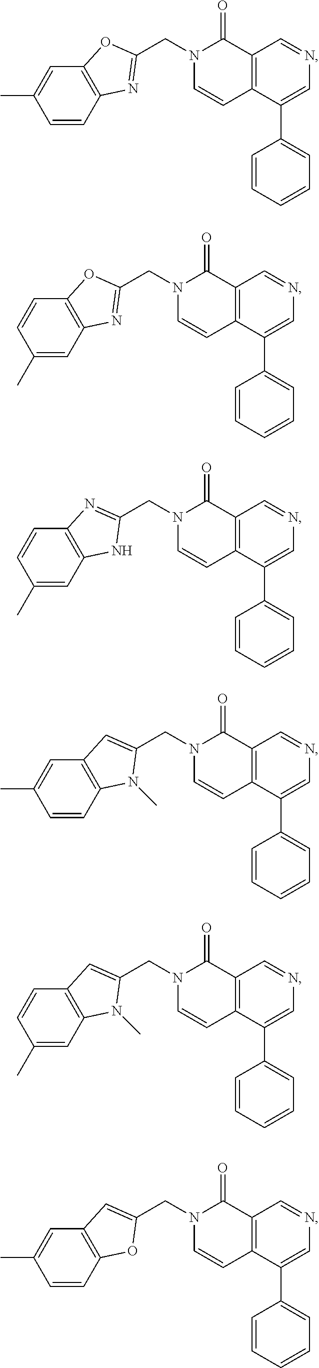 [SMILES: Cc1ccc2nc(Cn3ccc4c(-c5ccccc5)cncc4c3=O)oc2c1 ; Cc1ccc2oc(Cn3ccc4c(-c5ccccc5)cncc4c3=O)nc2c1 ; Cc1ccc2nc(Cn3ccc4c(-c5ccccc5)cncc4c3=O)[nH]c2c1 ; Cc1ccc2c(c1)cc(Cn1ccc3c(-c4ccccc4)cncc3c1=O)n2C ; Cc1ccc2cc(Cn3ccc4c(-c5ccccc5)cncc4c3=O)n(C)c2c1 ; Cc1ccc2oc(Cn3ccc4c(-c5ccccc5)cncc4c3=O)cc2c1]

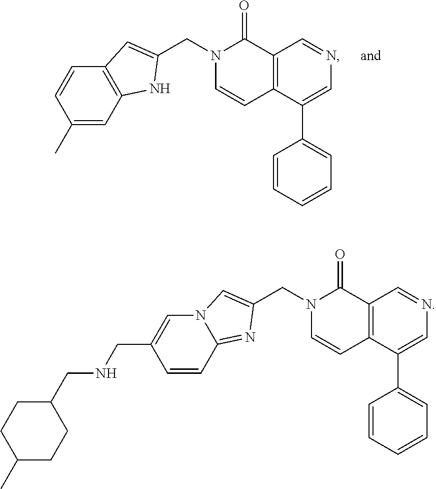 [SMILES: Cc1ccc2cc(Cn3ccc4c(-c5ccccc5)cncc4c3=O)[nH]c2c1 ; CC1CCC(CNCc2ccc3nc(Cn4ccc5c(-c6ccccc6)cncc5c4=O)cn3c2)CC1]

Embodiment I-90. A compound, or a pharmaceutically acceptable salt thereof, selected from the group consisting of

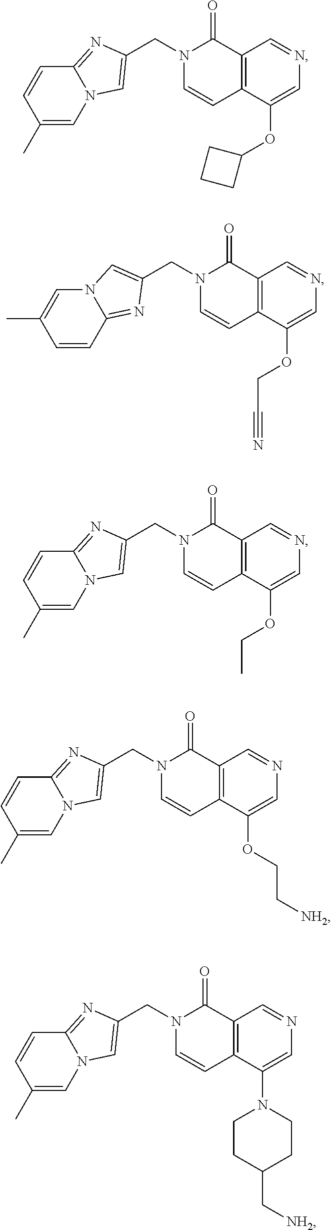

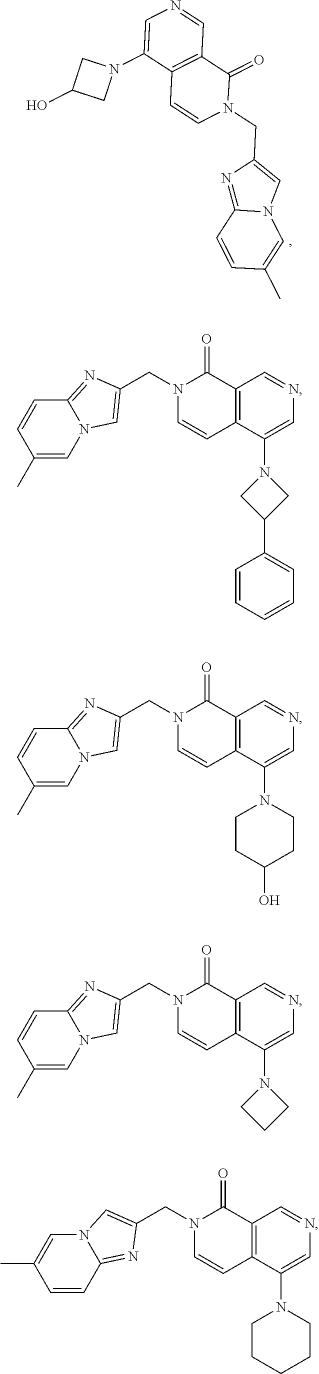 [SMILES: Cc1ccc2nc(Cn3ccc4c(N5CC(O)C5)cncc4c3=O)cn2c1 ; Cc1ccc2nc(Cn3ccc4c(N5CC(c6ccccc6)C5)cncc4c3=O)cn2c1 ; Cc1ccc2nc(Cn3ccc4c(N5CCC(O)CC5)cncc4c3=O)cn2c1 ; Cc1ccc2nc(Cn3ccc4c(N5CCC5)cncc4c3=O)cn2c1 ; Cc1ccc2nc(Cn3ccc4c(N5CCCCC5)cncc4c3=O)cn2c1]

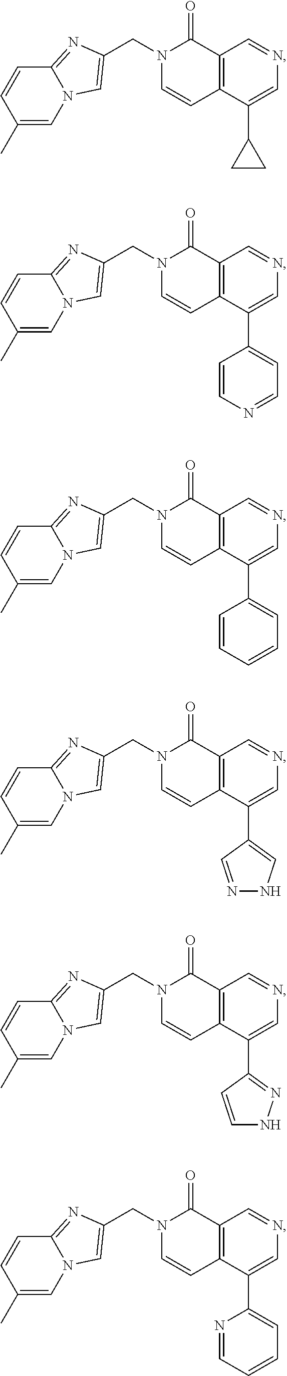 [SMILES: Cc1ccc2nc(Cn3ccc4c(C5CC5)cncc4c3=O)cn2c1 ; Cc1ccc2nc(Cn3ccc4c(-c5ccncc5)cncc4c3=O)cn2c1 ; Cc1ccc2nc(Cn3ccc4c(-c5ccccc5)cncc4c3=O)cn2c1 ; Cc1ccc2nc(Cn3ccc4c(-c5cn[nH]c5)cncc4c3=O)cn2c1 ; Cc1ccc2nc(Cn3ccc4c(-c5cc[nH]n5)cncc4c3=O)cn2c1 ; Cc1ccc2nc(Cn3ccc4c(-c5ccccn5)cncc4c3=O)cn2c1]

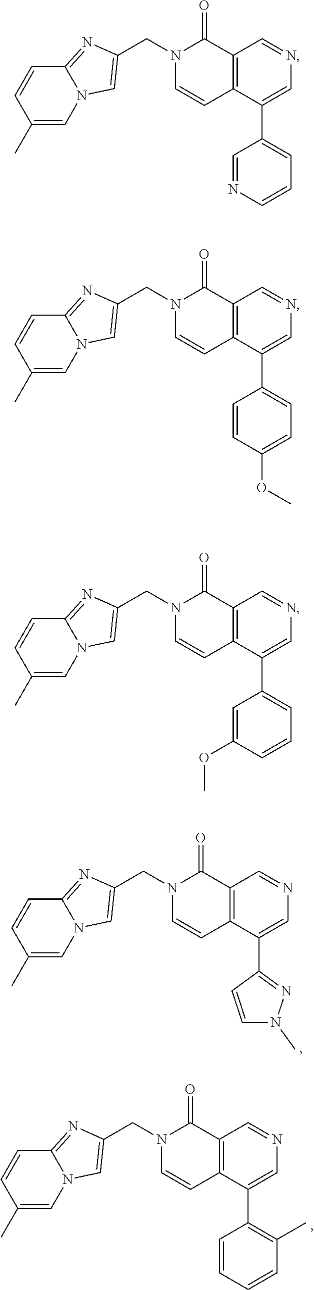 [SMILES: Cc1ccc2nc(Cn3ccc4c(-c5cccnc5)cncc4c3=O)cn2c1 ; COc1ccc(-c2cncc3c(=O)n(Cc4cn5cc(C)ccc5n4)ccc23)cc1 ; COc1cccc(-c2cncc3c(=O)n(Cc4cn5cc(C)ccc5n4)ccc23)c1 ; Cc1ccc2nc(Cn3ccc4c(-c5ccn(C)n5)cncc4c3=O)cn2c1 ; Cc1ccc2nc(Cn3ccc4c(-c5ccccc5C)cncc4c3=O)cn2c1]

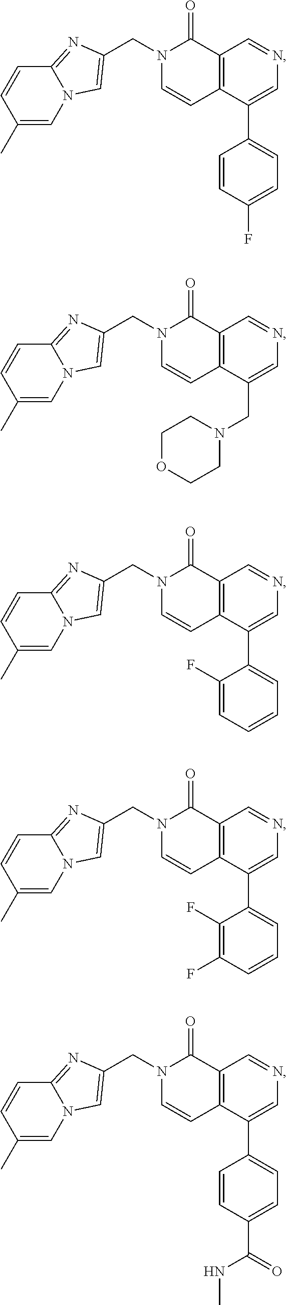 [SMILES: Cc1ccc2nc(Cn3ccc4c(-c5ccc(F)cc5)cncc4c3=O)cn2c1 ; Cc1ccc2nc(Cn3ccc4c(CN5CCOCC5)cncc4c3=O)cn2c1 ; Cc1ccc2nc(Cn3ccc4c(-c5ccccc5F)cncc4c3=O)cn2c1 ; Cc1ccc2nc(Cn3ccc4c(-c5cccc(F)c5F)cncc4c3=O)cn2c1 ; CNC(=O)c1ccc(-c2cncc3c(=O)n(Cc4cn5cc(C)ccc5n4)ccc23)cc1]

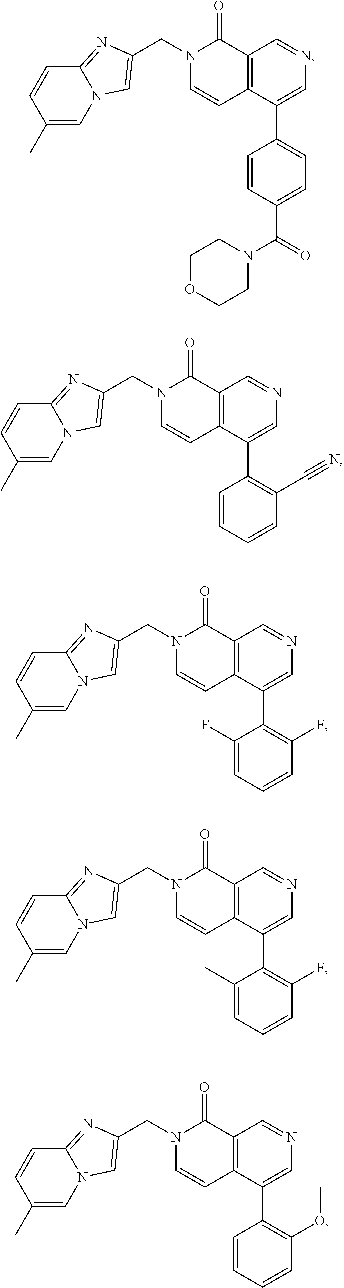 [SMILES: Cc1ccc2nc(Cn3ccc4c(-c5ccc(C(=O)N6CCOCC6)cc5)cncc4c3=O)cn2c1 ; Cc1ccc2nc(Cn3ccc4c(-c5ccccc5C#N)cncc4c3=O)cn2c1 ; Cc1ccc2nc(Cn3ccc4c(-c5c(F)cccc5F)cncc4c3=O)cn2c1 ; Cc1ccc2nc(Cn3ccc4c(-c5c(C)cccc5F)cncc4c3=O)cn2c1 ; COc1ccccc1-c1cncc2c(=O)n(Cc3cn4cc(C)ccc4n3)ccc12]

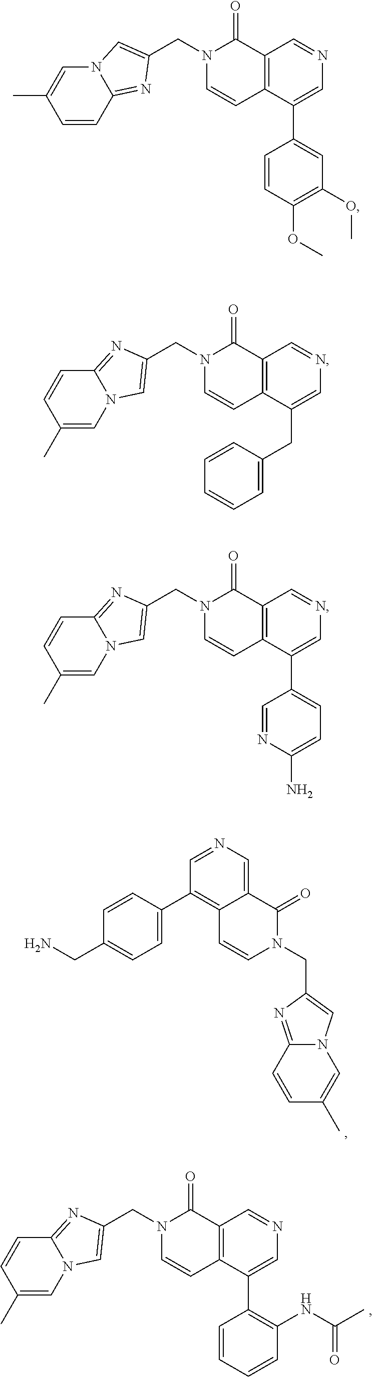

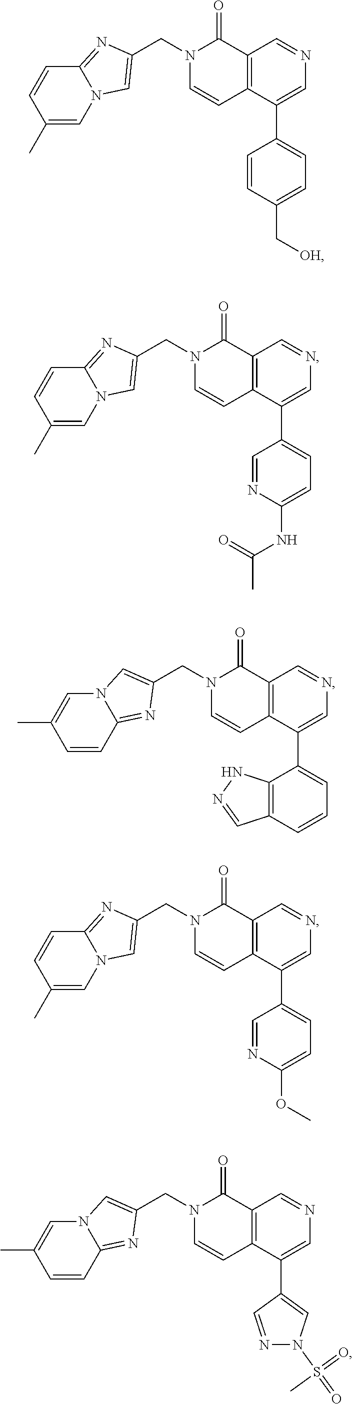

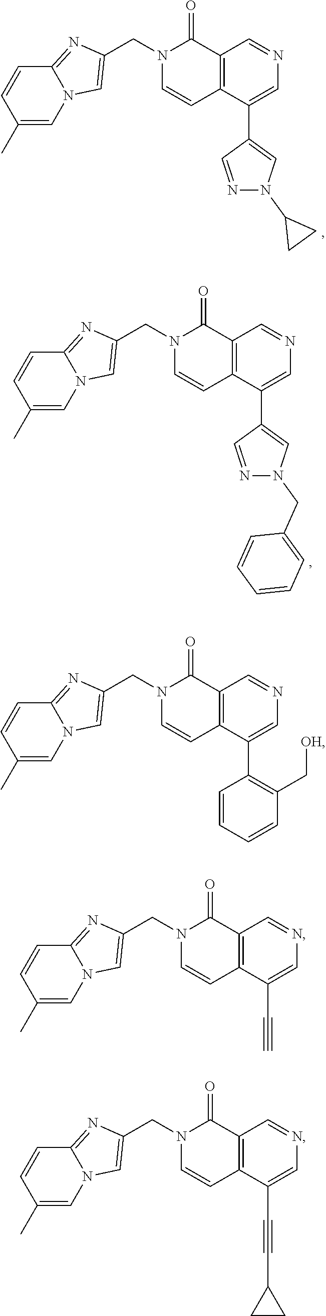 [SMILES: Cc1ccc2nc(Cn3ccc4c(-c5cnn(C6CC6)c5)cncc4c3=O)cn2c1 ; Cc1ccc2nc(Cn3ccc4c(-c5cnn(Cc6ccccc6)c5)cncc4c3=O)cn2c1 ; Cc1ccc2nc(Cn3ccc4c(-c5ccccc5CO)cncc4c3=O)cn2c1 ; C#Cc1cncc2c(=O)n(Cc3cn4cc(C)ccc4n3)ccc12 ; Cc1ccc2nc(Cn3ccc4c(C#CC5CC5)cncc4c3=O)cn2c1]

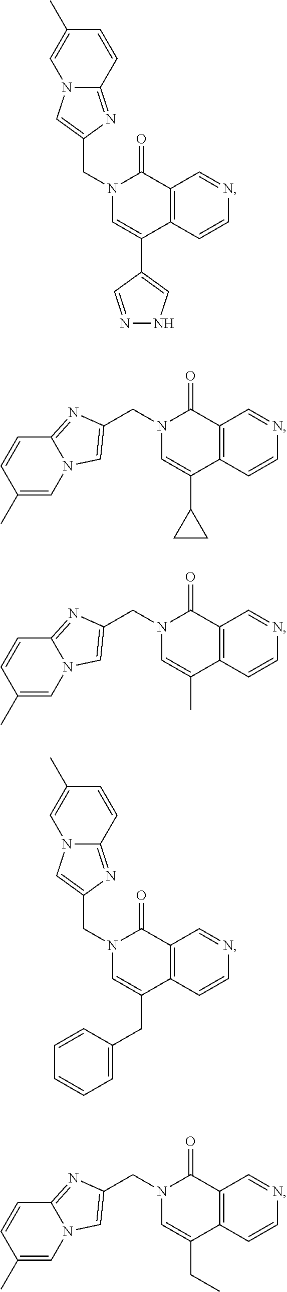

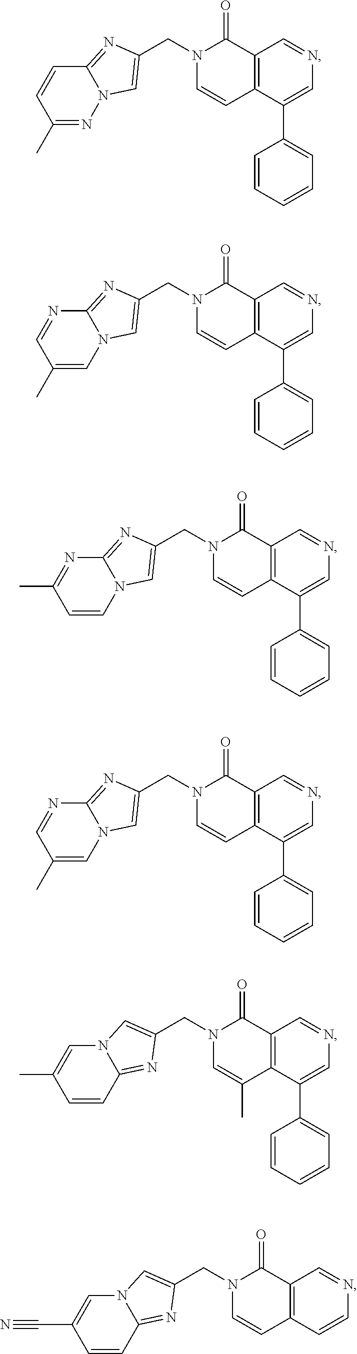 [SMILES: Cc1ccc2nc(Cn3ccc4c(-c5ccccc5)cncc4c3=O)cn2n1 ; Cc1cnc2nc(Cn3ccc4c(-c5ccccc5)cncc4c3=O)cn2c1 ; Cc1ccn2cc(Cn3ccc4c(-c5ccccc5)cncc4c3=O)nc2n1 ; Cc1cnc2nc(Cn3ccc4c(-c5ccccc5)cncc4c3=O)cn2c1 ; Cc1ccc2nc(Cn3cc(C)c4c(-c5ccccc5)cncc4c3=O)cn2c1 ; N#Cc1ccc2nc(Cn3ccc4ccncc4c3=O)cn2c1]

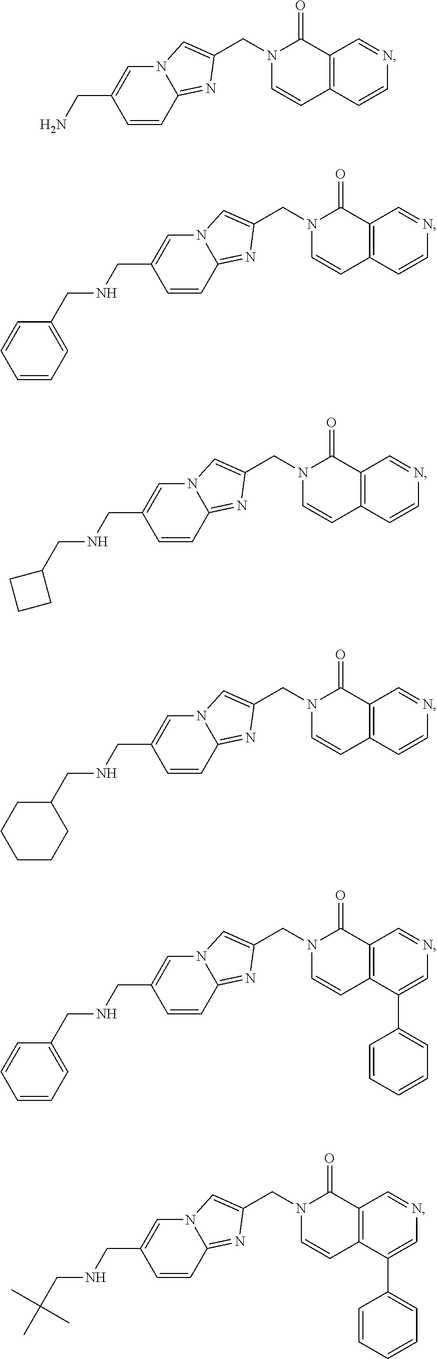 [SMILES: NCc1ccc2nc(Cn3ccc4ccncc4c3=O)cn2c1 ; O=c1c2cnccc2ccn1Cc1cn2cc(CNCc3ccccc3)ccc2n1 ; O=c1c2cnccc2ccn1Cc1cn2cc(CNCC3CCC3)ccc2n1 ; O=c1c2cnccc2ccn1Cc1cn2cc(CNCC3CCCCC3)ccc2n1 ; O=c1c2cncc(-c3ccccc3)c2ccn1Cc1cn2cc(CNCc3ccccc3)ccc2n1 ; CC(C)(C)CNCc1ccc2nc(Cn3ccc4c(-c5ccccc5)cncc4c3=O)cn2c1]

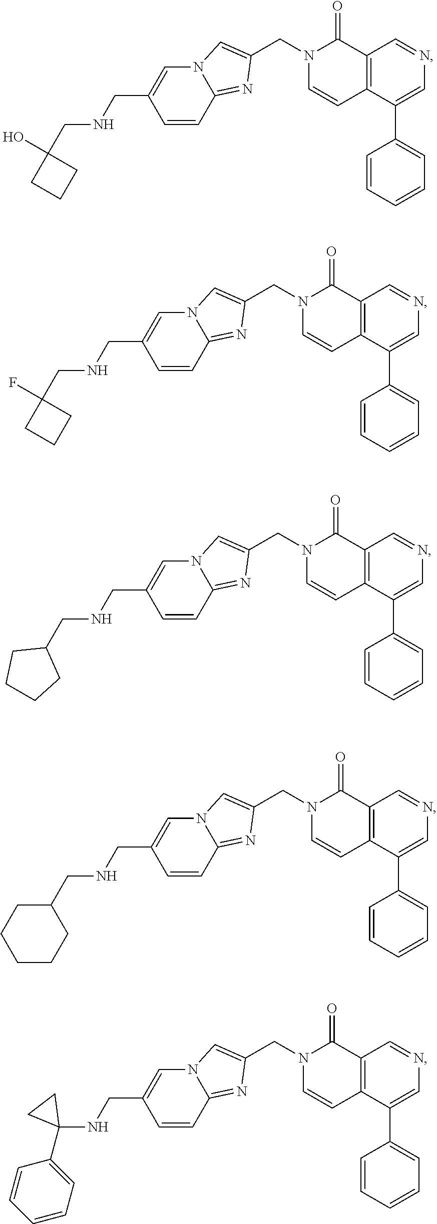 [SMILES: O=c1c2cncc(-c3ccccc3)c2ccn1Cc1cn2cc(CNCC3(O)CCC3)ccc2n1 ; O=c1c2cncc(-c3ccccc3)c2ccn1Cc1cn2cc(CNCC3(F)CCC3)ccc2n1 ; O=c1c2cncc(-c3ccccc3)c2ccn1Cc1cn2cc(CNCC3CCCC3)ccc2n1 ; O=c1c2cncc(-c3ccccc3)c2ccn1Cc1cn2cc(CNCC3CCCCC3)ccc2n1 ; O=c1c2cncc(-c3ccccc3)c2ccn1Cc1cn2cc(CNC3(c4ccccc4)CC3)ccc2n1]

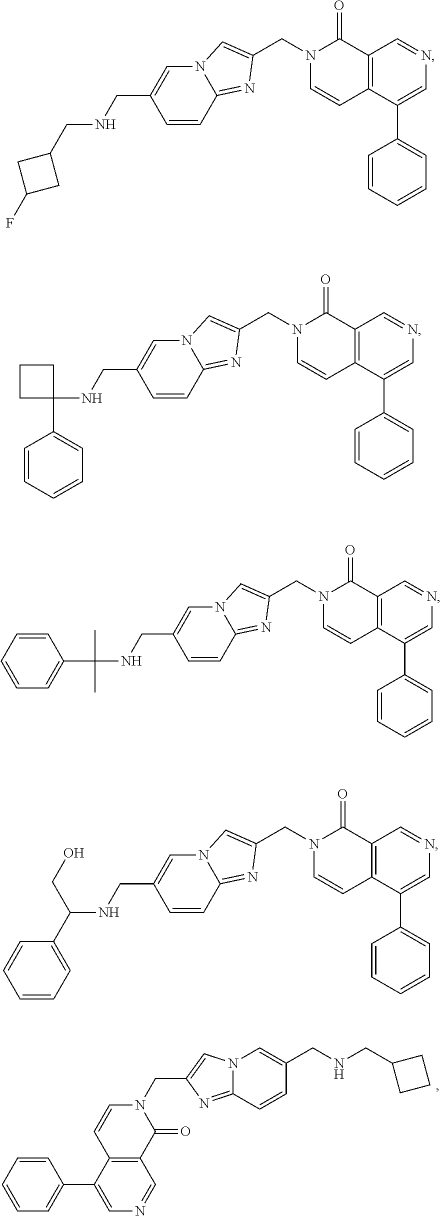 [SMILES: O=c1c2cncc(-c3ccccc3)c2ccn1Cc1cn2cc(CNCC3CC(F)C3)ccc2n1 ; O=c1c2cncc(-c3ccccc3)c2ccn1Cc1cn2cc(CNC3(c4ccccc4)CCC3)ccc2n1 ; CC(C)(NCc1ccc2nc(Cn3ccc4c(-c5ccccc5)cncc4c3=O)cn2c1)c1ccccc1 ; O=c1c2cncc(-c3ccccc3)c2ccn1Cc1cn2cc(CNC(CO)c3ccccc3)ccc2n1 ; O=c1c2cncc(-c3ccccc3)c2ccn1Cc1cn2cc(CNCC3CCC3)ccc2n1]

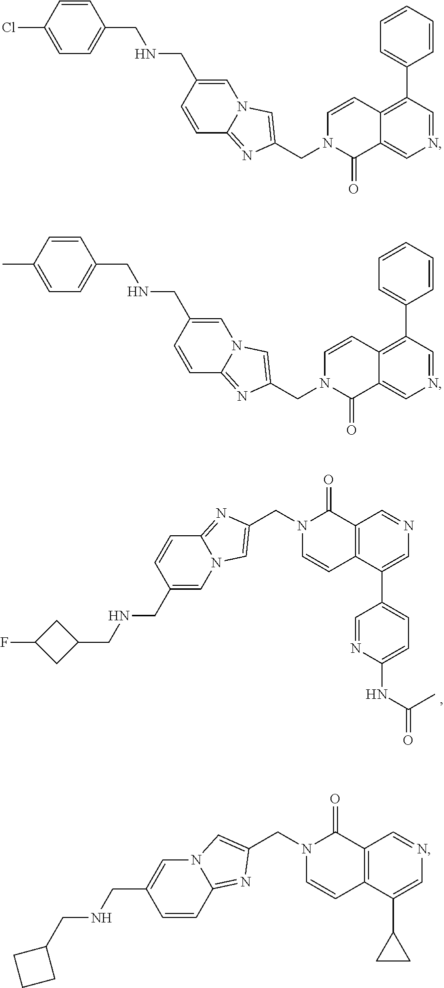 [SMILES: O=c1c2cncc(-c3ccccc3)c2ccn1Cc1cn2cc(CNCc3ccc(Cl)cc3)ccc2n1 ; Cc1ccc(CNCc2ccc3nc(Cn4ccc5c(-c6ccccc6)cncc5c4=O)cn3c2)cc1 ; CC(=O)Nc1ccc(-c2cncc3c(=O)n(Cc4cn5cc(CNCC6CC(F)C6)ccc5n4)ccc23)cn1 ; O=c1c2cncc(C3CC3)c2ccn1Cc1cn2cc(CNCC3CCC3)ccc2n1]

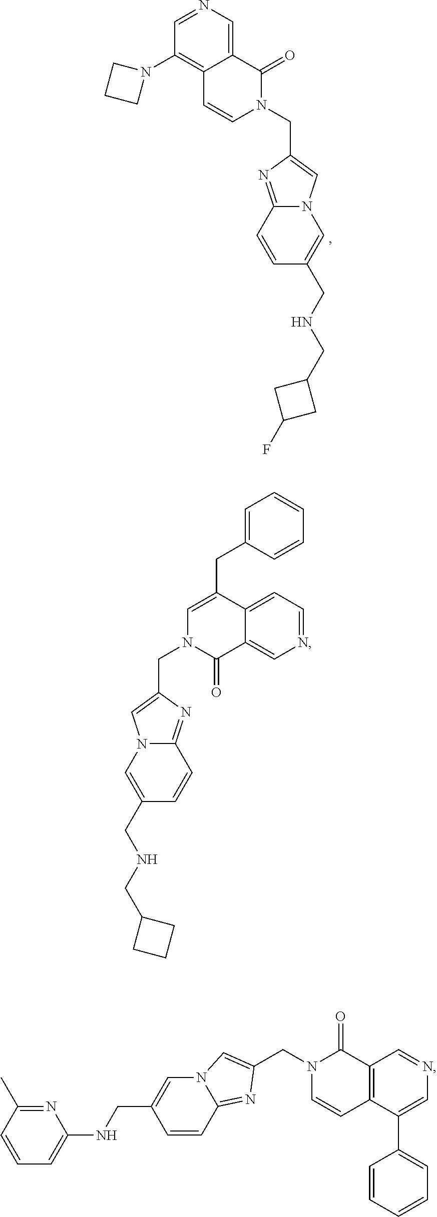

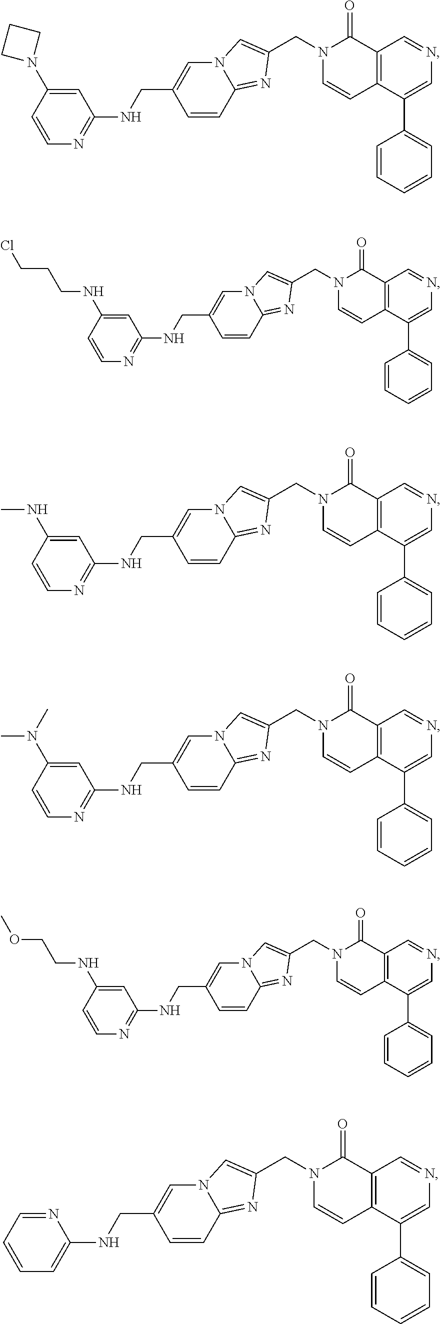 [SMILES: O=c1c2cncc(-c3ccccc3)c2ccn1Cc1cn2cc(CNc3cc(N4CCC4)ccn3)ccc2n1 ; O=c1c2cncc(-c3ccccc3)c2ccn1Cc1cn2cc(CNc3cc(NCCCCl)ccn3)ccc2n1 ; CNc1ccnc(NCc2ccc3nc(Cn4ccc5c(-c6ccccc6)cncc5c4=O)cn3c2)c1 ; CN(C)c1ccnc(NCc2ccc3nc(Cn4ccc5c(-c6ccccc6)cncc5c4=O)cn3c2)c1 ; COCCNc1ccnc(NCc2ccc3nc(Cn4ccc5c(-c6ccccc6)cncc5c4=O)cn3c2)c1 ; O=c1c2cncc(-c3ccccc3)c2ccn1Cc1cn2cc(CNc3ccccn3)ccc2n1]

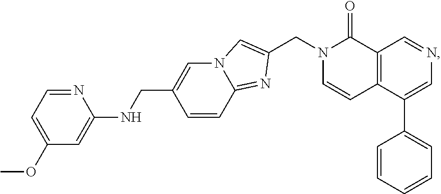 [SMILES: COc1ccnc(NCc2ccc3nc(Cn4ccc5c(-c6ccccc6)cncc5c4=O)cn3c2)c1]

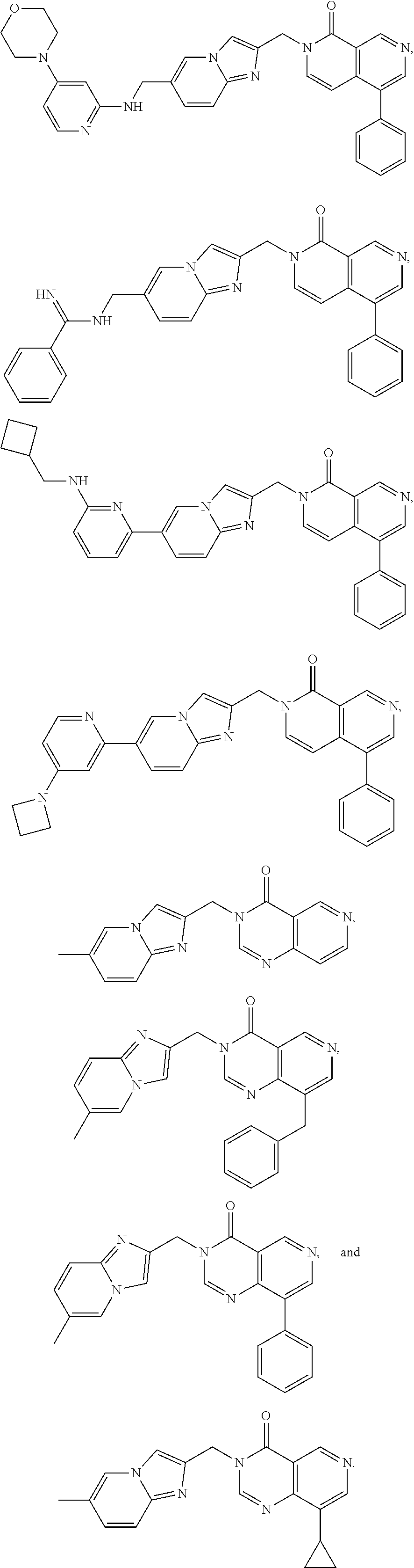

Embodiment I-91. A compound, or a pharmaceutically acceptable salt thereof, selected from the group consisting of

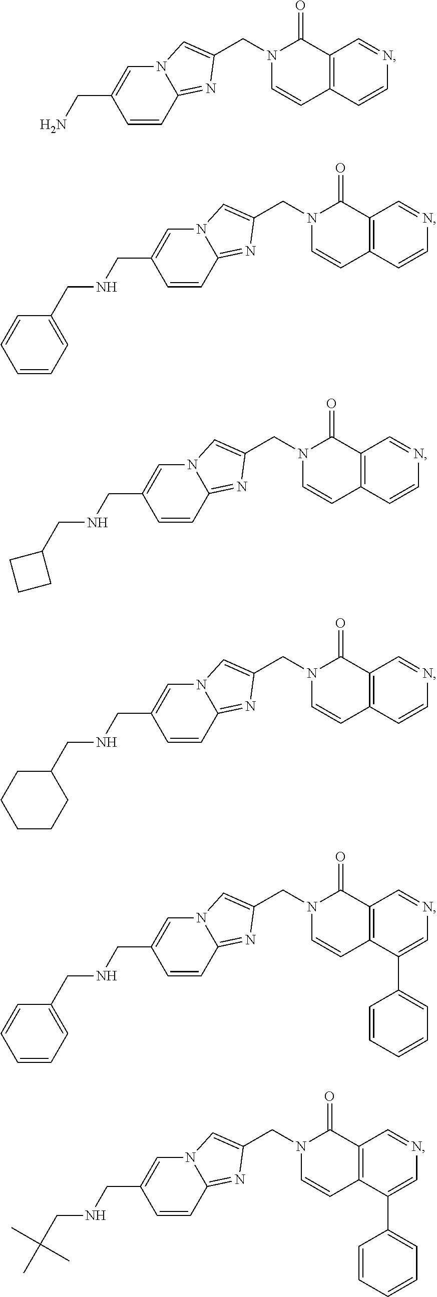 [SMILES: NCc1ccc2nc(Cn3ccc4ccncc4c3=O)cn2c1 ; O=c1c2cnccc2ccn1Cc1cn2cc(CNCc3ccccc3)ccc2n1 ; O=c1c2cnccc2ccn1Cc1cn2cc(CNCC3CCC3)ccc2n1 ; O=c1c2cnccc2ccn1Cc1cn2cc(CNCC3CCCCC3)ccc2n1 ; O=c1c2cncc(-c3ccccc3)c2ccn1Cc1cn2cc(CNCc3ccccc3)ccc2n1 ; CC(C)(C)CNCc1ccc2nc(Cn3ccc4c(-c5ccccc5)cncc4c3=O)cn2c1]

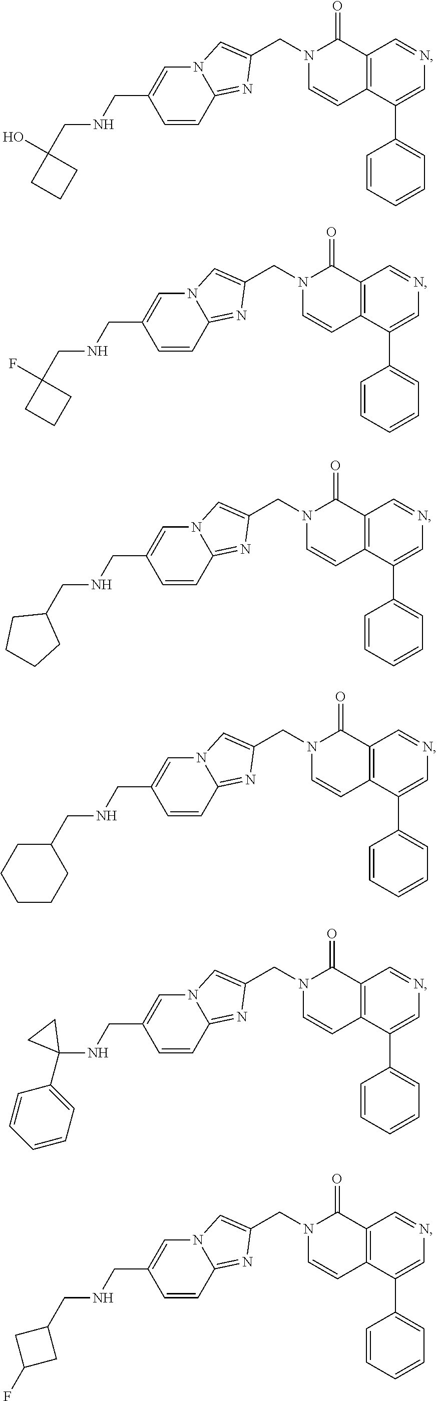 [SMILES: O=c1c2cncc(-c3ccccc3)c2ccn1Cc1cn2cc(CNCC3(O)CCC3)ccc2n1 ; O=c1c2cncc(-c3ccccc3)c2ccn1Cc1cn2cc(CNCC3(F)CCC3)ccc2n1 ; O=c1c2cncc(-c3ccccc3)c2ccn1Cc1cn2cc(CNCC3CCCC3)ccc2n1 ; O=c1c2cncc(-c3ccccc3)c2ccn1Cc1cn2cc(CNCC3CCCCC3)ccc2n1 ; O=c1c2cncc(-c3ccccc3)c2ccn1Cc1cn2cc(CNC3(c4ccccc4)CC3)ccc2n1 ; O=c1c2cncc(-c3ccccc3)c2ccn1Cc1cn2cc(CNCC3CC(F)C3)ccc2n1]

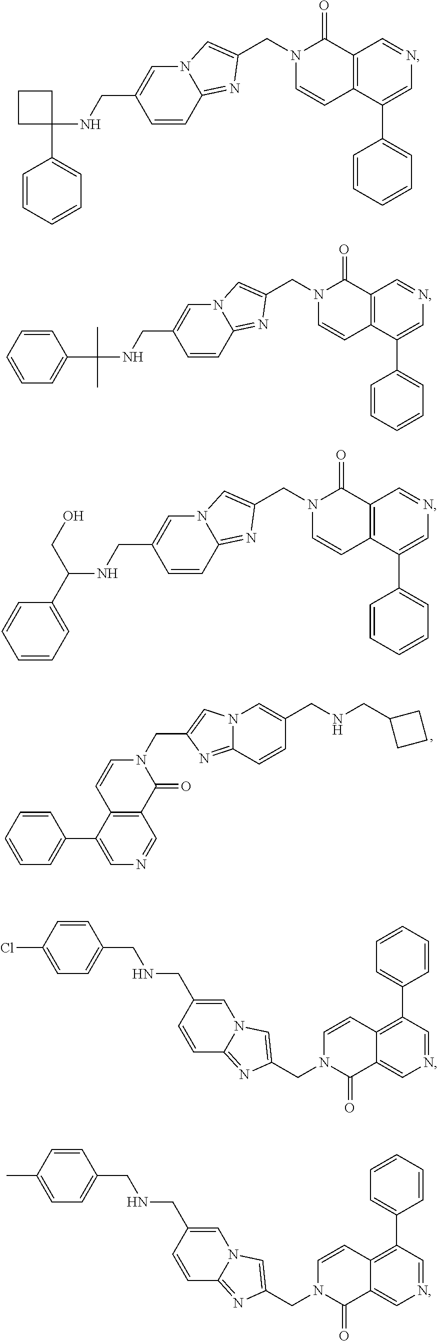 [SMILES: O=c1c2cncc(-c3ccccc3)c2ccn1Cc1cn2cc(CNC3(c4ccccc4)CCC3)ccc2n1 ; CC(C)(NCc1ccc2nc(Cn3ccc4c(-c5ccccc5)cncc4c3=O)cn2c1)c1ccccc1 ; O=c1c2cncc(-c3ccccc3)c2ccn1Cc1cn2cc(CNC(CO)c3ccccc3)ccc2n1 ; O=c1c2cncc(-c3ccccc3)c2ccn1Cc1cn2cc(CNCC3CCC3)ccc2n1 ; O=c1c2cncc(-c3ccccc3)c2ccn1Cc1cn2cc(CNCc3ccc(Cl)cc3)ccc2n1 ; Cc1ccc(CNCc2ccc3nc(Cn4ccc5c(-c6ccccc6)cncc5c4=O)cn3c2)cc1]

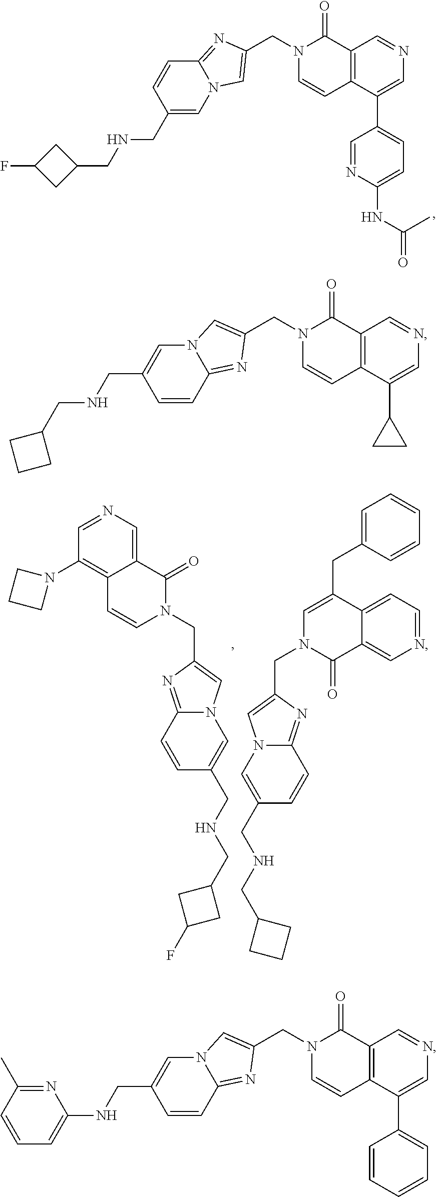

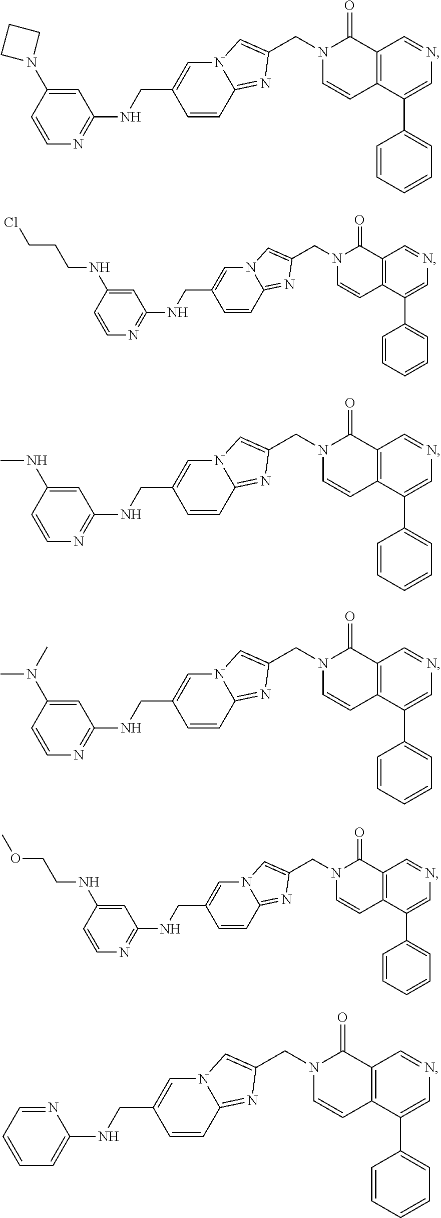 [SMILES: O=c1c2cncc(-c3ccccc3)c2ccn1Cc1cn2cc(CNc3cc(N4CCC4)ccn3)ccc2n1 ; O=c1c2cncc(-c3ccccc3)c2ccn1Cc1cn2cc(CNc3cc(NCCCCl)ccn3)ccc2n1 ; CNc1ccnc(NCc2ccc3nc(Cn4ccc5c(-c6ccccc6)cncc5c4=O)cn3c2)c1 ; CN(C)c1ccnc(NCc2ccc3nc(Cn4ccc5c(-c6ccccc6)cncc5c4=O)cn3c2)c1 ; COCCNc1ccnc(NCc2ccc3nc(Cn4ccc5c(-c6ccccc6)cncc5c4=O)cn3c2)c1 ; O=c1c2cncc(-c3ccccc3)c2ccn1Cc1cn2cc(CNc3ccccn3)ccc2n1]

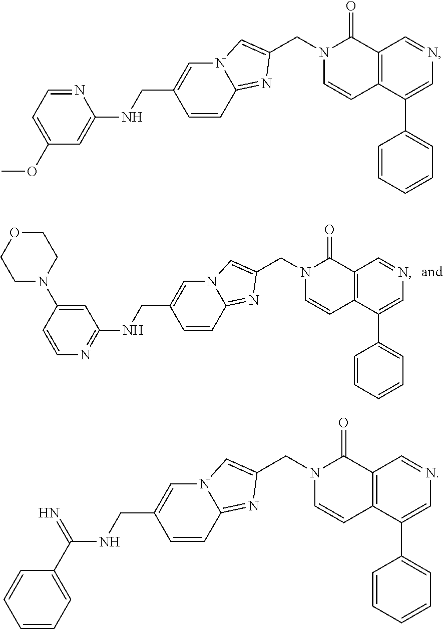

EmbodimentI-92. A compound of the structure

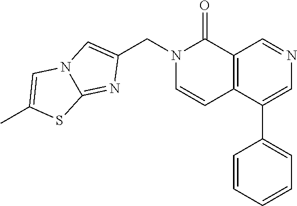 [SMILES: Cc1cn2cc(Cn3ccc4c(-c5ccccc5)cncc4c3=O)nc2s1]

or a pharmaceutically acceptable salt thereof.

Embodiment I-93. A compound, or a pharmaceutically acceptable salt thereof, selected from the group consisting of

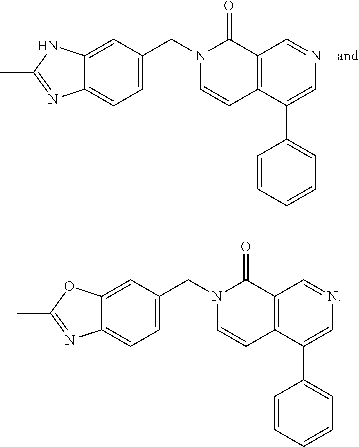 [SMILES: Cc1nc2ccc(Cn3ccc4c(-c5ccccc5)cncc4c3=O)cc2[nH]1 ; Cc1nc2ccc(Cn3ccc4c(-c5ccccc5)cncc4c3=O)cc2o1]

Embodiment I-94. A compound, or a pharmaceutically acceptable salt thereof, selected from the group consisting of

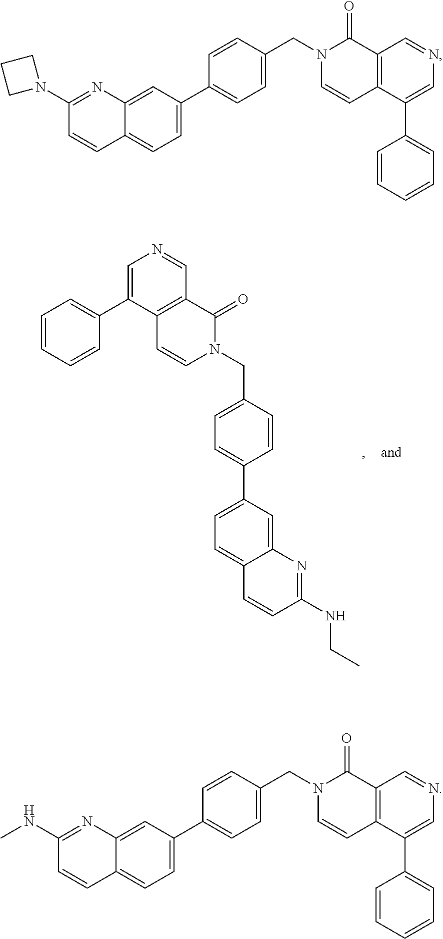

Embodiment I-95. A compound, or a pharmaceutically acceptable salt thereof, selected from the group consisting of:
5-{6,6-difluoro-2-azaspiro[3.3]heptan-2-yl}-2-({6-[(4,4-dimethylpiperidin-1-yl)methyl]imidazo[1,2-a]pyridin-2-yl}methyl)-1,2-dihydro-2,7-naphthyridin-1-one;
5-(4,4-difluoropiperidin-1-yl)-2-({6-[(4,4-dimethylpiperidin-1-yl)methyl]imidazo[1,2-a]pyridin-2-yl}methyl)-1,2-dihydro-2,7-naphthyridin-1-one;
2-({6-[(4,4-dimethylpiperidin-1-yl)methyl]imidazo[1,2-a]pyridin-2-yl}methyl)-5-(morpholin-4-yl)-1,2-dihydro-2,7-naphthyridin-1-one;
2-({6-[(4,4-dimethylpiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one;
5-((1S,4S)-2-oxa-5-azabicyclo[2.2.1]heptan-5-yl)-2-((6-((4,4-dimethylpiperidin-1-yl)methyl)imidazo[1,2-a]pyridin-2-yl)methyl)-2,7-naphthyridin-1(2H)-one;
2-({6-[(4,4-dimethylpiperidin-1-yl)methyl]imidazo[1,2-a]pyridin-2-yl}methyl)-5-[(1R,4R)-2-oxa-5-azabicyclo[2.2.1]heptan-5-yl]-1,2-dihydro-2,7-naphthyridin-1-one;
2-({6-[(4,4-dimethylpiperidin-1-yl)methyl]imidazo[1,2-a]pyridin-2-yl}methyl)-5-{hexahydro-1H-furo[3,4-c]pyrrol-5-yl}-1,2-dihydro-2,7-naphthyridin-1-one;
2-({6-[(4,4-dimethylpiperidin-1-yl)methyl]imidazo[1,2-a]pyridin-2-yl}methyl)-5-[3-(morpholin-4-yl)azetidin-1-yl]-1,2-dihydro-2,7-naphthyridin-1-one;
   5-{2,7-diazaspiro[3.5]nonan-2-yl}-2-({6-[(4,4-dimethylpiperidin-1-yl)methyl]imidazo[1,2-a]pyridin-2-yl}methyl)-1,2-dihydro-2,7-naphthyridin-1-one;
5-(3,4-dihydro-2H-1,4-benzoxazin-4-yl)-2-({6-[(4,4-dimethylpiperidin-1-yl)methyl]imidazo[1,2-a]pyridin-2-yl}methyl)-1,2-dihydro-2,7-naphthyridin-1-one;
2-({6-[(4,4-dimethylpiperidin-1-yl)methyl]imidazo[1,2-a]pyridin-2-yl}methyl)-5-phenyl-1,2-dihydro-2,7-naphthyridin-1-one;
2-({6-[(4,4-dimethylpiperidin-1-yl)methyl]imidazo[1,2-a]pyridin-2-yl}methyl)-5-(quinolin-5-yl)-1,2-dihydro-2,7-naphthyridin-1-one;
   4-[7-({6-[(4,4-dimethylpiperidin-1-yl)methyl]imidazo[1,2-a]pyridin-2-yl}methyl)-8-oxo-7,8-dihydro-2,7-naphthyridin-4-yl]benzene-1-sulfonamide;
2-((6-((4,4-dimethylpiperidin-1-yl)methyl)imidazo[1,2-a]pyridin-2-yl)methyl)-5-(pyridin-4-ylethynyl)-2,7-naphthyridin-1(2H)-one;
   5-{3-[(dimethylamino)methyl]azetidin-1-yl}-2-({6-[(4,4-dimethylpiperidin-1-yl)methyl]imidazo[1,2-a]pyridin-2-yl}methyl)-1,2-dihydro-2,7-naphthyridin-1-one;
2-({6-[(4,4-dimethylpiperidin-1-yl)methyl]imidazo[1,2-a]pyridin-2-yl}methyl)-5-[3-(4-methylpiperazin-1-yl)azetidin-1-yl]-1,2-dihydro-2,7-naphthyridin-1-one;
   2-{[6-({6-azaspiro[2.5]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
   2-{[6-({2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-((6-((7-azaspiro[3.5]nonan-7-yl)methyl)imidazo[1,2-a]pyridin-2-yl)methyl)-5-(7-oxa-2-azaspiro[3.5]nonan-2-yl)-2,7-naphthyridin-1(2H)-one;
   5-{7-oxa-2-azaspiro[3.5]nonan-2-yl}-2-{[6-({2-oxa-7-azaspiro[4.4]nonan-7-yl}methyl)imidazo[1,2-a]pyridin-2-yl]methyl}-1,2-dihydro-2,7-naphthyridin-1-one;
   5-{7-oxa-2-azaspiro[3.5]nonan-2-yl}-2-{[6-({6-oxa-3-azabicyclo[3.1.1]heptan-3-yl}methyl)imidazo[1,2-a]pyridin-2-yl]methyl}-1,2-dihydro-2,7-naphthyridin-1-one;
   5-{7-oxa-2-azaspiro[3.5]nonan-2-yl}-2-[(6-{[4-(trifluoromethyl)piperidin-1-yl]methyl}imidazo[1,2-a]pyridin-2-yl)methyl]-1,2-dihydro-2,7-naphthyridin-1-one;
   2-{[6-({8-methyl-3,8-diazabicyclo[3.2.1]octan-3-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
4-methyl-1-({2-[(5-{7-oxa-2-azaspiro[3.5]nonan-2-yl}-1-oxo-1,2-dihydro-2,7-naphthyridin-2-yl)methyl]imidazo[1,2-a]pyridin-6-yl}methyl)piperidine-4-carbonitrile;
   2-{[6-({6,6-difluoro-2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
   2-{[6-({6-fluoro-2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
   2-{[6-({2-azaspiro[4.4]nonan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-({6-[(4-methylpiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one;
2-({6-[(4,4-difluoropiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one;
   2-{[6-({2,2-difluoro-6-azaspiro[3.4]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
   2-{[6-({1,1-difluoro-6-azaspiro[2.5]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
   5-{6,6-difluoro-2-azaspiro[3.3]heptan-2-yl}-2-{[6-({[(3-fluorocyclobutyl)methyl]amino}methyl)imidazo[1,2-a]pyridin-2-yl]methyl}-1,2-dihydro-2,7-naphthyridin-1-one;
   2-{[6-({[(3-fluorocyclobutyl)methyl]amino}methyl)imidazo[1,2-a]pyridin-2-yl]methyl}-5-{2-oxa-6-azaspiro[3.3]heptan-6-yl}-1,2-dihydro-2,7-naphthyridin-1-one;
   2-{[6-({[(3-fluorocyclobutyl)methyl]amino}methyl)imidazo[1,2-a]pyridin-2-yl]methyl}-5-{7-oxa-2-azaspiro[3.5]nonan-2-yl}-1,2-dihydro-2,7-naphthyridin-1-one;
   2-{[6-({[(3-fluorocyclobutyl)methyl]amino}methyl)imidazo[1,2-a]pyridin-2-yl]methyl}-5-{6-oxa-2-azaspiro[3.5]nonan-2-yl}-1,2-dihydro-2,7-naphthyridin-1-one;
   5-{7,7-difluoro-2-azaspiro[3.5]nonan-2-yl}-2-{[6-({[(3-fluorocyclobutyl)methyl]amino}methyl)imidazo[1,2-a]pyridin-2-yl]methyl}-1,2-dihydro-2,7-naphthyridin-1-one;
   5-{2,2-difluoro-6-azaspiro[3.4]octan-6-yl}-2-{[6-({[(3-fluorocyclobutyl)methyl]amino}methyl)imidazo[1,2-a]pyridin-2-yl]methyl}-1,2-dihydro-2,7-naphthyridin-1-one;
   2-{[6-({[(3-fluorocyclobutyl)methyl]amino}methyl)imidazo[1,2-a]pyridin-2-yl]methyl}-5-[3-(4-fluoropiperidin-1-yl)azetidin-1-yl]-1,2-dihydro-2,7-naphthyridin-1-one;
   2-{[6-({[(3-fluorocyclobutyl)methyl]amino}methyl)imidazo[1,2-a]pyridin-2-yl]methyl}-5-[3-(pyridin-4-yl)azetidin-1-yl]-1,2-dihydro-2,7-naphthyridin-1-one;
   2-{[6-({[(3-fluorocyclobutyl)methyl]amino}methyl)imidazo[1,2-a]pyridin-2-yl]methyl}-5-[3-(4-fluorophenyl)azetidin-1-yl]-1,2-dihydro-2,7-naphthyridin-1-one;
2-((6-((((3-fluorobicyclo[1.1.1]pentan-1-yl)methyl)amino)methyl)imidazo[1,2-a]pyridin-2-yl)methyl)-5-(6-oxa-2-azaspiro[3.4]octan-2-yl)-2,7-naphthyridin-1(2H)-one;
   2-[(6-{[({3-fluorobicyclo[1.1.1]pentan-1-yl}methyl)amino]methyl}imidazo[1,2-a]pyridin-2-yl)methyl]-5-{7-oxa-2-azaspiro[3.5]nonan-2-yl}-1,2-dihydro-2,7-naphthyridin-1-one;
   2-[(6-{[({3-fluorobicyclo[1.1.1]pentan-1-yl}methyl)amino]methyl}imidazo[1,2-a]pyridin-2-yl)methyl]-5-{6-oxa-2-azaspiro[3.5]nonan-2-yl}-1,2-dihydro-2,7-naphthyridin-1-one;
   2-[(6-{[({3-fluorobicyclo[1.1.1]pentan-1-yl}methyl)amino]methyl}imidazo[1,2-a]pyridin-2-yl)methyl]-5-(2-fluorophenyl)-1,2-dihydro-2,7-naphthyridin-1-one;
   4-{7-[(6-{[({3-fluorobicyclo[1.1.1]pentan-1-yl}methyl)amino]methyl}imidazo[1,2-a]pyridin-2-yl)methyl]-8-oxo-7,8-dihydro-2,7-naphthyridin-4-yl}benzamide;
5-cyclobutyl-2-{[6-({[(3-fluorocyclobutyl)methyl]amino}methyl)imidazo[1,2-a]pyridin-2-yl]methyl}-1,2-dihydro-2,7-naphthyridin-1-one; and
   3-[(6-{[({3-fluorobicyclo[1.1.1]pentan-1-yl}methyl)amino]methyl}imidazo[1,2-a]pyridin-2-yl)methyl]-8-{2-oxa-6-azaspiro[3.3]heptan-6-yl}pyrido[3,4-d]pyridazin-4-one.

Embodiment I-96. A pharmaceutical composition comprising a compound of any one of embodiments I-1 to I-95, or a pharmaceutically acceptable salt thereof, and a pharmaceutically acceptable carrier.

Embodiment I-97. A compound of any one of embodiments I-1 to I-95, or a pharmaceutically acceptable salt thereof, for use in medicine.

Embodiment I-98. A compound of any one of embodiments I-1 to I-95, or a pharmaceutically acceptable salt thereof, for use in the treatment of a disorder that is responsive to modulation or inhibition of METTL3.

Embodiment I-99. Use of a compound of any one of embodiments I-1 to I-95, or a pharmaceutically acceptable salt thereof, in the treatment of a disorder that is responsive to modulation or inhibition of METTL3.

Embodiment I-100. A compound of any one of embodiments I-1 to I-95, or a pharmaceutically acceptable salt thereof, for use in the manufacture of a medicament for the treatment of a disorder that is responsive to modulation or inhibition of METTL3.

Embodiment I-101. A method of treating a disorder in a subject in need thereof, wherein the disorder is mediated by METTL3, comprising administering to the subject an effective amount of a compound of any one of embodiments I-1 to I-95, or a pharmaceutically acceptable salt thereof.

Embodiment I-102. The compound of embodiment I-98 or I-100, the use of embodiment I-998, or the method of embodiment I-101, wherein the disease, disorder, or condition is selected from the group consisting of neurological diseases, viral diseases, cancer, and pre-cancerous syndromes.

Embodiment I-103. The compound of embodiment I-98 or I-100, the use of embodiment I-99, or the method of embodiment I-101 or I-102, wherein the disorder is cancer and the cancer is selected from the group consisting of blood cancer, pancreatic cancer, gastric cancer, colorectal cancer, ovarian cancer, endometrial cancer, bladder cancer, breast cancer, brain cancer, melanoma, lung cancer, and liver cancer.

Embodiment I-104. The compound of embodiment I-98 or I-100, the use of embodiment I-99, or the method of any one of embodiments I-101 to 1-103, wherein the disorder is leukemia.

Embodiment I-106. The compound of embodiment I-98 or I-100, the use of embodiment I-99, or the method of any one of embodiments I-101 to 1-104, wherein the disorder is acute myeloid leukemia (AML).

Another embodiment provides a compound of Formula (VII), or a pharmaceutically acceptable salt thereof, having the structure:

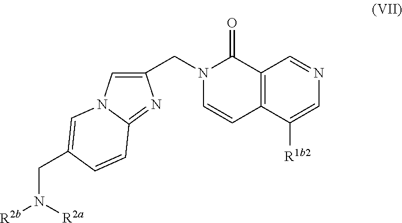

wherein:
R^{1b2} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b2} is unsubstituted or substituted with one or more substituents selected from the group consisting of C₁₋₆alkyl, halo, —CN, —(CH₂)ₘN(R^{1e})₂, —(CH₂)ₘOH, —C(O)R^{1e}, —C(O)N(R^{1e})₂, —(CH₂)ₘCOOH, aryl, heteroaryl, heterocyclyl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b2} is unsubstituted or substituted with one or more substituents selected from the group consisting of aryl, heteroaryl, heterocyclyl, and cycloalkyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
R^{2a} is H or C₁₋₆alkyl;
wherein the C₁₋₆alkyl of R^{2a} is unsubstituted or substituted with one or more substituents selected from the group consisting of halogen, —OH, —CN, —N(R^{2h})₂, alkoxy, cycloalkyl, halocycloalkyl, hydroxycycloalkyl, and heterocyclyl;
R^{2b} is selected from the group consisting of C₁₋₆alkyl, heterocyclyl, cycloalkyl, heterocyclylalkyl, and cycloalkylalkyl;
wherein the C₁₋₆alkyl, heterocyclyl, cycloalkyl, heterocyclylalkyl, and cycloalkylalkyl of R^{2b} is unsubstituted or substituted with one or more substituents selected from the group consisting of C₁₋₆alkyl, C₁₋₆haloalkyl, halogen, —OH, —CN, —N(R^{2h})₂, alkoxy, aryl, haloaryl, cycloalkyl, halocycloalkyl, hydroxycycloalkyl, and heterocyclyl; or R^{2a} and R^{2b} combine to form a 5-8-membered heterocyclyl;
each R^{2h} is independently H, C₁₋₆alkyl, or —(CH₂)·OCH₃; and
n is 1, 2, 3, 4, 5, or 6.

Another embodiment provides the compound of Formula (VII), or a pharmaceutically acceptable salt thereof, wherein R^{2a} is H.

Another embodiment provides the compound of Formula (VII), or a pharmaceutically acceptable salt thereof, wherein R^{2b} is cycloalkylalkyl.

Another embodiment provides the compound of Formula (VII), or a pharmaceutically acceptable salt thereof, wherein R^{2b} is cycloalkylalkyl substituted with one or more substituents selected from C₁₋₆haloalkyl, halogen, or —CN.

Another embodiment provides the compound of Formula (VII), or a pharmaceutically acceptable salt thereof, wherein —N(R^{2a})(R^{2b}) is selected from:

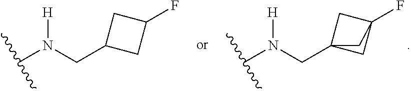 [SMILES: *NCC1CC(F)C1 ; *NCC12CC(F)(C1)C2]

Another embodiment provides the compound of Formula (VII), or a pharmaceutically acceptable salt thereof, wherein R^{2a} is C₁₋₆alkyl.

Another embodiment provides the compound of Formula (VII), or a pharmaceutically acceptable salt thereof, wherein R^{2b} is C₁₋₆alkyl.

Another embodiment provides the compound of Formula (VII), or a pharmaceutically acceptable salt thereof, wherein R^{2b} is C₁₋₆alkyl substituted with one or more substituents selected from C₁₋₆haloalkyl, halogen, or —CN.

Another embodiment provides the compound of Formula (VII), or a pharmaceutically acceptable salt thereof, wherein R^{2b} is heterocyclylalkyl.

Another embodiment provides the compound of Formula (VII), or a pharmaceutically acceptable salt thereof, wherein R^{2b} is heterocyclylalkyl substituted with one or more substituents selected from C₁₋₆alkyl, C₁₋₆haloalkyl, halogen, or —CN.

Another embodiment provides the compound of Formula (VII), or a pharmaceutically acceptable salt thereof, wherein R^{2b} is cycloalkylalkyl.

Another embodiment provides the compound of Formula (VII), or a pharmaceutically acceptable salt thereof, wherein R^{2b} is cycloalkylalkyl substituted with one or more substituents selected from C₁₋₆alkyl, C₁₋₆haloalkyl, halogen, or —CN.

Another embodiment provides the compound of Formula (VII), or a pharmaceutically acceptable salt thereof, wherein R^{2a} and R^{2b} combine to form a 5-8-membered heterocyclyl.

Another embodiment provides the compound of Formula (VII), or a pharmaceutically acceptable salt thereof, wherein —N(R^{2a})(R^{2b}) is selected from:

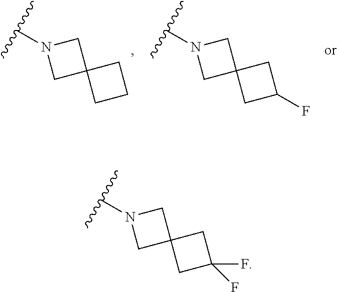 [SMILES: *N1CC2(CCC2)C1 ; *N1CC2(CC(F)C2)C1 ; *N1CC2(C1)CC(F)(F)C2]

Another embodiment provides the compound of Formula (VII), or a pharmaceutically acceptable salt thereof, wherein —N(R^{2a})(R^{2b}) is selected from:

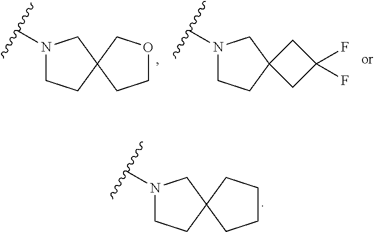

Another embodiment provides the compound of Formula (VII), or a pharmaceutically acceptable salt thereof, wherein —N(R^{2a})(R^{2b}) is selected from:

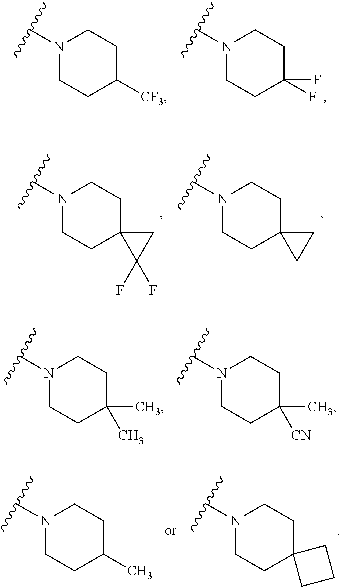

Another embodiment provides the compound of Formula (VII), or a pharmaceutically acceptable salt thereof, wherein —N(R^{2a})(R^{2b}) is selected from:

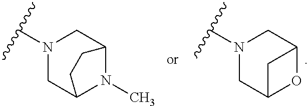

Another embodiment provides the compound of Formula (VII), or a pharmaceutically acceptable salt thereof, wherein R^{1b2} is heterocyclyl.

Another embodiment provides the compound of Formula (VII), or a pharmaceutically acceptable salt thereof, wherein R^{1b2} is selected from:

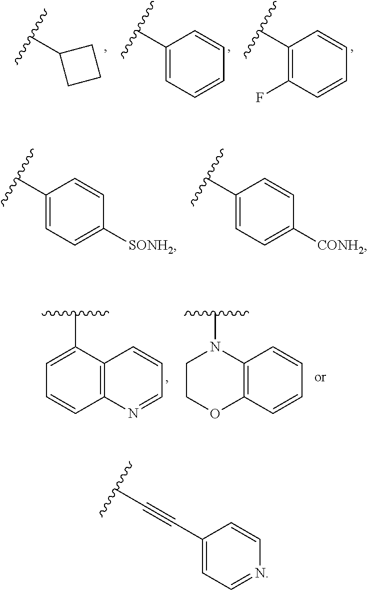

Another embodiment provides the compound of Formula (VII), or a pharmaceutically acceptable salt thereof, wherein R^{1b2} is selected from:

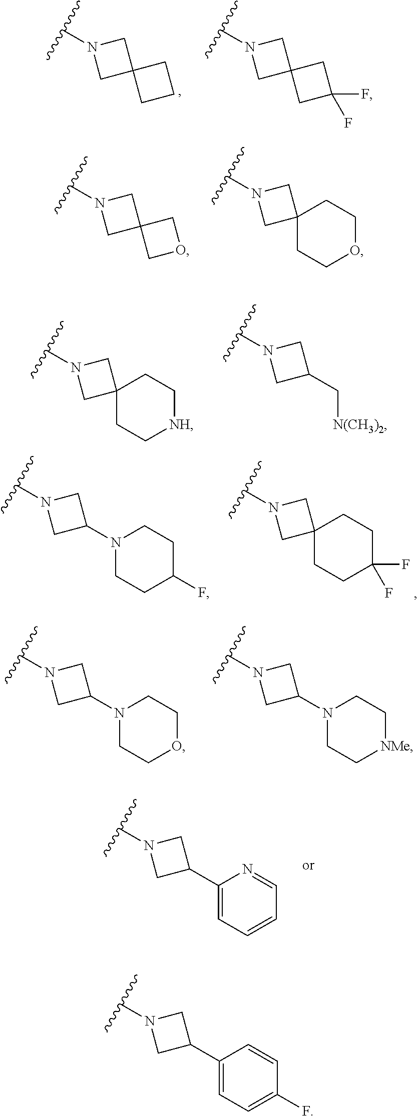

Another embodiment provides the compound of Formula (VII), or a pharmaceutically acceptable salt thereof, wherein R^{1b2} is selected from:

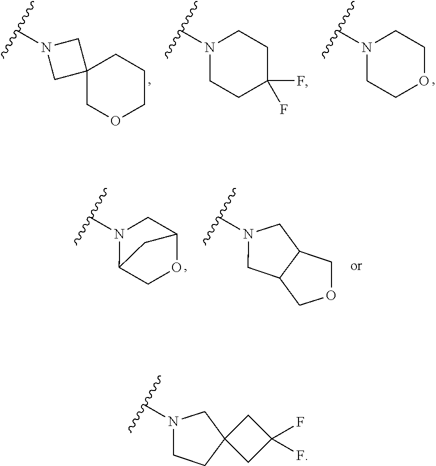

One embodiment provides a pharmaceutical composition comprising a compound of of Formula (VII), or a pharmaceutically acceptable salt thereof, and a pharmaceutically acceptable carrier.

One embodiment provides a compound of of Formula (VII), or a pharmaceutically acceptable salt thereof, for use in medicine.

One embodiment provides a compound of of Formula (VII), or a pharmaceutically acceptable salt thereof, for use in the treatment of a disorder that is responsive to modulation or inhibition of METTL3.

One embodiment provides a use of a compound of of Formula (VII), or a pharmaceutically acceptable salt thereof, in the treatment of a disorder that is responsive to modulation or inhibition of METTL3. Another embodiment provides the use wherein the disease, disorder, or condition is selected from the group consisting of neurological diseases, viral diseases, cancer, and pre-cancerous syndromes. Another embodiment provides the use wherein the disorder is cancer and the cancer is selected from the group consisting of blood cancer, pancreatic cancer, gastric cancer, colorectal cancer, ovarian cancer, endometrial cancer, bladder cancer, breast cancer, brain cancer, melanoma, lung cancer, and liver cancer. The ues wherein the disorder is leukemia. Another embodiment provides the use wherein the disorder is acute myeloid leukemia (AML).

One embodiment provides a compound of of Formula (VII), or a pharmaceutically acceptable salt thereof, for use in the manufacture of a medicament for the treatment of a disorder that is responsive to modulation or inhibition of METTL3.

One embodiment provides a method of treating a disorder in a subject in need thereof, wherein the disorder is mediated by METTL3, comprising administering to the subject an effective amount of a compound of of Formula (VII), or a pharmaceutically acceptable salt thereof. Another embodiment provides the method wherein the disease, disorder, or condition is selected from the group consisting of neurological diseases, viral diseases, cancer, and pre-cancerous syndromes. Another embodiment provides the method wherein the disorder is cancer and the cancer is selected from the group consisting of blood cancer, pancreatic cancer, gastric cancer, colorectal cancer, ovarian cancer, endometrial cancer, bladder cancer, breast cancer, brain cancer, melanoma, lung cancer, and liver cancer. Another embodiment provides the method wherein the disorder is leukemia. The method wherein the disorder is acute myeloid leukemia (AML).

### Examples

The following examples are provided to illustrate the present disclosure, and should not be construed as limiting thereof. In these examples, all parts and percentages are by weight, unless otherwise noted.

### Synthetic Example 1: Synthesis of 5-cyclobutoxy-2-((6-methylimidazo[1,2-a]pyridin-2-yl)methyl)-2,7-naphthyridin-1(2H)-one. (Compound No. 1)

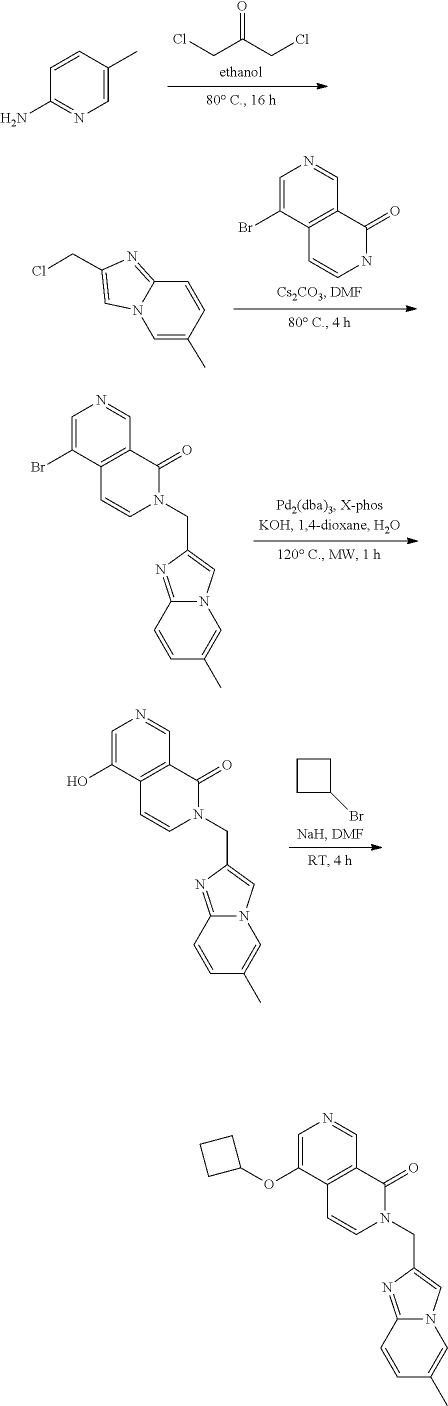

Step 1: 1,3-dichloropropan-2-one (6.46 g 50.9 mmol) was added to a stirred solution of 5-methylpyridin-2-amine (5.00 g, 46.2 mmol) in ethanol (100 mL) at room temperature. The reaction mixture was heated at 80° C. for 16 h, cooled to room temperature, concentrated in vacuo and purified by column chromatography (SiO₂, 5% MeOH/DCM) to afford 2-(chloromethyl)-6-methylimidazo[1,2-a]pyridine (2.0 g yield: 24%) as a pink solid. MS: m/z: 181 [M+H]⁺

Step 2: Caesium carbonate (1.15 g, 3.56 mmol) was added to a solution of 5-bromo-1,2-dihydro-2,7-naphthyridin-1-one(400 mg, 1.78 mmol) and 2-(chloromethyl)-6-methylimidazo[1,2-a]pyridine (321 mg, 1.78 mmol) in DMF (5 mL) and the reaction mixture was heated at 80° C. for 4 h. After cooling to room temperature, the reaction mixture was diluted with cold water (20 mL), and the precipitated solid was filtered and dried to give 5-bromo-2-({6-methylimidazo[1,2-a]pyridin-2-yl}methyl)-1,2-dihydro-2,7-naphthyridin-1-one (600 mg, yield: 91%) as a pale brown solid. MS: m/z: 370 [M+H]⁺

Step 3: A stirred solution of 5-bromo-2-({6-methylimidazo[1,2-a]pyridin-2-yl}methyl)-1,2,3,4-tetrahydro-2,7-naphthyridin-1-one(50 mg, 135 μmol) in 1,4-dioxane(5 mL) at room temperature was purged with argon for 10 min, followed by the addition of KOH (35.7 mg, 637 μmol), tris(1,5-diphenylpenta-1,4-dien-3-one) dipalladium(11.7 mg 12.7 μmol) and dicyclohexyl[2′,4′,6′-tris(propan-2-yl)-[1,1′-biphenyl]-2-yl]phosphane (30.4 mg, 63.7 μmol). The reaction mixture was irradiated in a microwave reactor at 120′ C for 1 h, cooled to room temperature, diluted with water (5 mL), neutralized with 2N HCl (pH 7), and extracted with 10% MeOH: DCM (60 mL×3). The combined organic layers were dried over sodium sulphate, filtered, and concentrated in vacuo to give 5-hydroxy-2-({6-methylimidazo[1,2-a]pyridin-2-yl}methyl)-1,2,3,4-tetrahydro-2,7-naphthyridin-1-one (15 mg, yield: 23%) as a white solid. MS: m/z: 307 [M+H]⁺

Step 4: 5-hydroxy-2-({6-methylimidazo[1,2-a]pyridin-2-yl}methyl)-1,2-dihydro-2,7-naphthyridin-1-one (30 mg, 97.9 μmol) was added to a suspension of 60% sodium hydride (10.4 mg, 262 μmol) in DMF (2 mL) at 0° C., and after 30 min bromocyclobutane (11.5 mg, 135 μmol) was added to the reaction mixture. After stirring at room temperature for 4 h, the reaction mixture was quenched with ice-cold water (5 mL), and extracted with 10% MeOH/DCM (60 mL). The organic layer was dried over sodium sulphate, filtered, concentrated in vacuo and purified by reverse phase prep-HPLC (Column: POROSHELL C18 3.0*50 mm, 2.7 μm; 30-100% CH3CN in H₂O) to give (5-cyclobutoxy-2-((6-methylimidazo[1,2-a]pyridin-2-yl)methyl)-2,7-naphthyridin-1(2H)-one (5 mg, yield: 14%) as an off-white solid. ¹H NMR (400 MHz, DMSO-d₆): δ 8.97 (s, 1H), 8.28 (d, J=0.8 Hz, 1H), 7.76 (s, 1H), 7.84 (d, J=7.2 Hz, 1H), 7.76 (s, 1H), 7.40 (d, J=9.2 Hz, 1H), 7.08 (dd, J=9.2, 1.6 Hz, 1H), 6.73 (dd, J=7.6, 0.4 Hz, 1H), 5.27 (s, 2H), 4.95 (p, J=7.2 Hz, 1H), 2.24 (s, 3H), 2.46-2.45 (m, 2H), 2.14-2.09 (m, 2H), 1.84-1.81 (m, 1H), 1.68-1.66 (m, 1H). MS: m/z: 361 [M+H]⁺

### Synthetic Example 2: Synthesis of 2-((7-((6-methylimidazo[1,2-a]pyridin-2-yl)methyl)-8-oxo-7,8-dihydro-2,7-naphthyridin-4-yl) oxy) acetonitrile. (Compound No. 2)

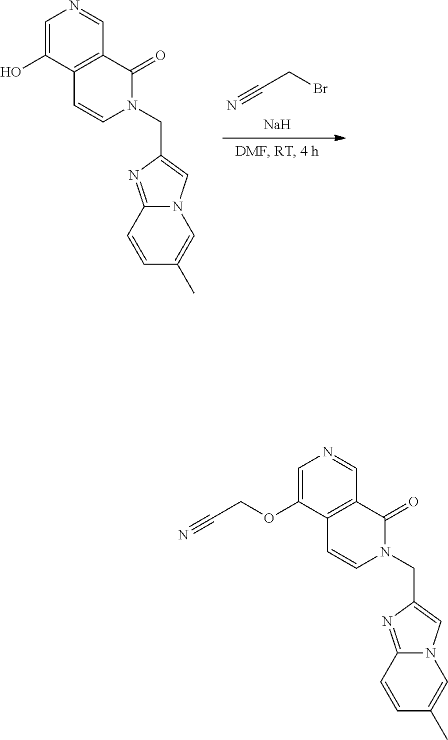

The title compound was prepared using the procedure described for example 1, step 4 with the following modification: the reaction was performed with 2-bromoacetonitrile to give 2-{[7-({6-methylimidazo[1,2-a]pyridin-2-yl}methyl)-8-oxo-7,8-dihydro-2,7-naphthyridin-4-yl]oxy}acetonitrile (yield: 21%) as an off-white solid. ¹H NMR (400 MHz, DMSO-d₆): δ 9.10 (d, J=0.7 Hz, 1H), 8.59 (s, 1H), 8.28 (t, J=1.2 Hz, 1H), 7.90 (d, J=7.6 Hz, 1H), 7.78 (s, 1H), 7.40 (d, J=9.2 Hz, 1H), 7.08 (dd, J=9.2, 1.6 Hz, 1H), 6.71 (dd, J=7.6, 0.8 Hz, 1H), 5.45 (s, 2H), 5.28 (s, 2H), 2.24 (d, J=1.1 Hz, 3H). MS: m/z 346 [M+H]⁺

### Synthetic Example 3: 5-ethoxy-2-((6-methylimidazo[1,2-a]pyridin-2-yl)methyl)-2,7-naphthyridin-1(2H)-one. (Compound No. 3)

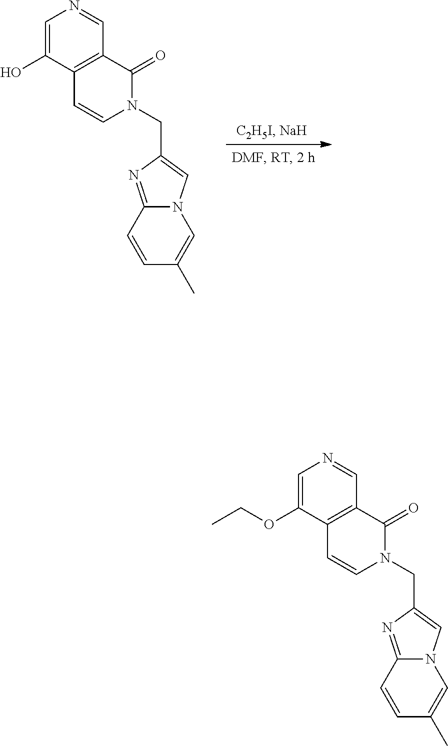

The title compound was prepared using the procedure described for example 1, step 4 with the following modification: the reaction was performed with ethyl bromide to give 5-ethoxy-2-({6-methylimidazo[1,2-a]pyridin-2-yl}methyl)-1,2-dihydro-2,7-naphthyridin-1-one (yield: 10%) as an off-white solid. ¹H NMR (400 MHz, DMSO-d₆): δ 8.98 (s, 1H), 8.43 (s, 1H), 8.28 (d, J=0.8 Hz, 1H), 7.83 (d, J=7.6 Hz, 1H), 7.76 (s, 1H), 7.39 (d, J=9.2 Hz, 1H), 7.08 (dd, J=9.2, 1.6 Hz, 1H), 6.72 (dd, J=7.6, 0.8 Hz, 1H), 5.27 (s, 2H), 4.27 (q, J=6.8 Hz, 2H), 2.24 (d, J=0.8 Hz, 3H), 1.42 (t, J=6.8 Hz, 3H). MS: m/z: 335 [M+H]⁺

### Synthetic Example 4: Synthesis of Synthesis of 5-(2-aminoethoxy)-2-((6-methylimidazo[1,2-a]pyridin-2-yl)methyl)- 2,7-naphthyridin-1(2H)-one.(Compound No. 4)

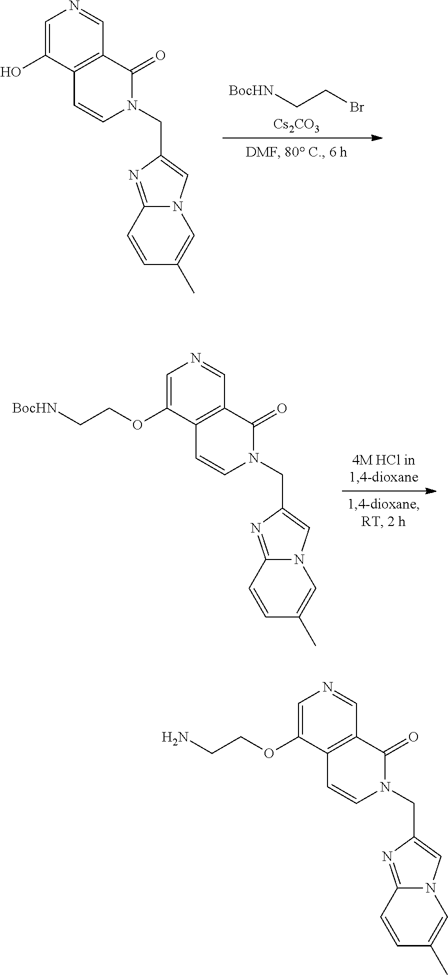

Step 1: Caesium carbonate (265 mg, 813 μmol) was added to a solution of 5-hydroxy-2-({6-methylimidazo[1,2-a]pyridin-2-yl}methyl)-1,2-dihydro-2,7-naphthyridin-1-one (0.1 g, 326 μmol) in DMF (4 mL), followed by the addition of tert-butyl N-(2-bromoethyl) carbamate (72.8 mg, 325 μmol). The reaction mixture was heated at 80° C. for 6 h, diluted with ice cold water (10 mL), and extracted with 10% MeOH/DCM (60 mL×2). The combined organic layers were dried over sodium sulphate, filtered, and concentrated in vacuo to give [(tert-butoxy) carbonyl](2-{[7-({6-methylimidazo[1,2-a]pyridin-2-yl}methyl)-8-oxo-7,8-dihydro-2,7-naphthyridin-4-yl]oxy}ethyl) aminyl (42 mg, yield: 34%) as a pale brown solid.

Step 2: A 4M HCl solution in dioxane (1 mL) was added dropwise to [(tert-butoxy) carbonyl](2-{[7-({6-methylimidazo[1,2-a]pyridin-2-yl}methyl)-8-oxo-7,8-dihydro-2,7-naphthyridin-4-yl]oxy}ethyl) aminyl (42 mg, 93.2 μmol) in 1,4-dioxane (5 mL) at 0° C. The reaction mixture was allowed to warm to room temperature for 2 h, concentrated in vacuo and purified by reverse phase prep HPLC (Column: POROSHELL C18 3.0*50 mm, 2.7 μm; 0.01-20% CH₃CN in H₂O) to give 5-(2-aminoethoxy)-2-({6-methylimidazo[1,2-a]pyridin-2-yl}methyl)-1,2-dihydro-2,7-naphthyridin-1-one (16 mg, yield: 50%) as an off-white solid. ¹H NMR (400 MHz, DMSO-d₆): δ 8.98 (s, 1H), 8.43 (s, 1H), 8.28 ((m, 1H), 7.83 (d, J=7.6 Hz, 1H), 7.75 (s, 1H), 7.39 (d, J=9.2 Hz, 1H), 7.08 (dd, J=9.2, 1.6 Hz, 1H), 6.84 (d, J=7.2 Hz, 1H), 5.27 (s, 2H), 4.15 (t, J=5.4 Hz, 2H), 2.96 (t, J=5.4 Hz, 2H), 2.24 (s, 3H). MS: m/z 350 [M+H]⁺

### Synthetic Example 5: Synthesis of 5-(4-(aminomethyl) piperidin-1-yl)-2-((6-methylimidazo[1,2-a]pyridin-2-yl)methyl)-2,7-naphthyridin-1(2H)-one. (Compound No. 5)

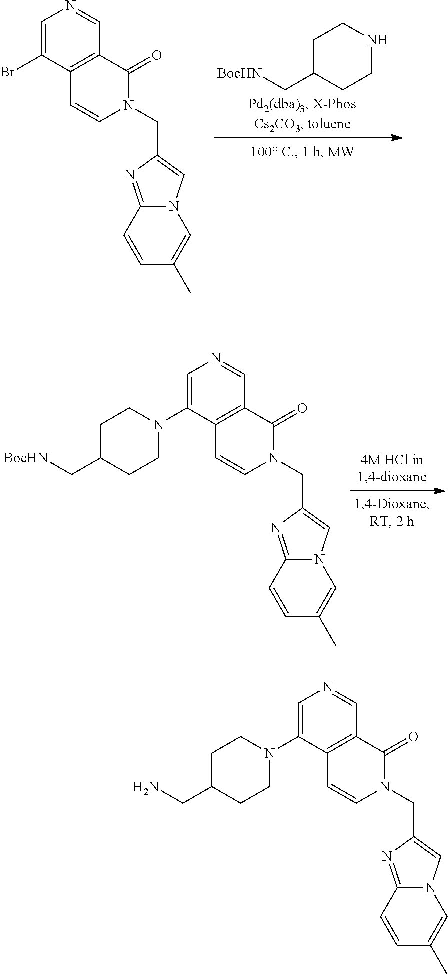

Step 1: A solution of 5-bromo-2-({6-methylimidazo[1,2-a]pyridin-2-yl}methyl)-1,2-dihydro-2,7-naphthyridin-1-one(100 mg, 271 μmol), caesium carbonate (265 mg, 813 μmol), and 4-({[(tert-butoxy)carbonyl]amino}methyl)piperidin-1-yl (69.3 mg, 325 μmol) in toluene (5 mL) was purged with argon for 10 min, followed by the addition of dicyclohexyl[2′,4′,6′-tris(propan-2-yl)-[1,1′-biphenyl]-2-yl]phosphane(63.9 mg, 135 μmol), and tris(1,5-diphenylpenta-1,4-dien-3-one)dipalladium (124 mg, 135 μmol). The reaction mixture was irradiated in a microwave reactor at 100° C. for 1 h, concentrated in vacuo and purified by column chromatography (SiO₂, 80% EtOAc/hexane) to give tert-butyl N-({1-[7-({6-methylimidazo[1,2-a]pyridin-2-yl}methyl)-8-oxo-7,8-dihydro-2,7-naphthyridin-4-yl]piperidin-4-yl}methyl)carbamate (40 mg, yield: 29%) as a pale brown solid. MS: m/z: 503 [M+H]⁺

Step 2: The title compound was prepared using the procedure described for example 4, step 2 with the following modification: the reaction was performed with tert-butyl N-({1-[7-({6-methylimidazo[1,2-a]pyridin-2-yl}methyl)-8-oxo-7,8-dihydro-2,7-naphthyridin-4-yl]piperidin-4-yl}methyl) carbamate (40 mg, 79.6 mol) and purified by reverse phase Prep HPLC (Column: POROSHELL C18 3.0*50 mm, 2.7 μm, 20-100% CH₃CN/H₂O) to afford 5-[4-(aminomethyl)piperidin-1-yl]-2-({6-methylimidazo[1,2-a]pyridin-2-yl}methyl)-1,2-dihydro-2,7-naphthyridin-1-one (8.2 mg, yield: 25%) as an off-white solid. ¹H NMR (400 MHz, DMSO-d₆) δ 9.04 (s, 1H), 8.39 (s, 1H), 8.28 (t, J=0.8 Hz, 1H), 7.83 (d, J=7.6 Hz, 1H), 7.77 (s, 1H), 7.40 (d, J=9.2 Hz, 1H), 7.08 (dd, J=9.2, 1.6 Hz, 1H), 6.60 (d, J=7.6 Hz, 1H), 5.26 (s, 2H), 3.28 (d, J=12 Hz, 2H), 2.75 (t, J=10.4 Hz, 2H), 2.57 (d, J=6.0 Hz, 2H), 2.24 (s, 3H), 1.84 (d, J=10 Hz, 2H), 1.38 (td, J=12.0, 3.2 Hz, 3H). MS: m/z: 403 [M+H]⁺

### Synthetic Example 6: Synthesis of 5-(4-(3-hydroxyazetidin-1-yl) piperidin-1-yl)-2-((6-methylimidazo[1,2-a]pyridin-2-yl)methyl)-2,7-naphthyridin-1(2H)-one. (Compound No. 6)

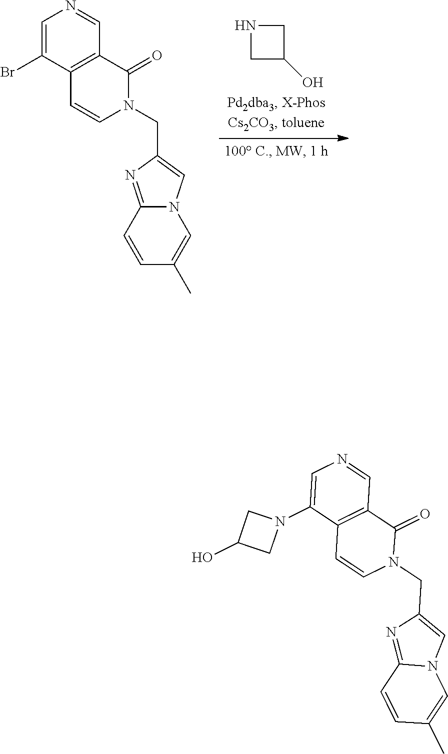 [SMILES: Cc1ccc2nc(Cn3ccc4c(Br)cncc4c3=O)cn2c1 ; OC1CNC1 ; Cc1ccc2nc(Cn3ccc4c(N5CC(O)C5)cncc4c3=O)cn2c1]

The title compound was prepared using the procedure described for example 5, step 1 with the following modification: the reaction was performed with 3-hydroxy azetidine to give 5-(4-(3-hydroxyazetidin-1-yl)piperidin-1-yl)-2-((6-methylimidazo[1,2-a]pyridin-2-yl)methyl)-2,7-naphthyridin-1(2H)-one (yield: 9%) as an off-white solid. ¹H NMR (400 MHz, DMSO-d₆): δ 8.81 (s, 1H), 8.29 (s, 1H), 7.90 (s, 1H), 7.77 (s, 1H), 7.68 (d, J=7.6 Hz, 1H), 7.40 (d, J=9.2 Hz, 1H), 7.08 (dd, J=9.2, 1.6 Hz, 1H), 6.58 (d, J=7.6 Hz, 1H), 5.68 (d, J=6.4 Hz, 1H), 5.24 (s, 2H), 4.58 (p, J=6.4 Hz, 1H), 4.38 (t, J=7.6 Hz, 2H), 3.80-3.77 ((m, 2H), 2.24 (s, 3H). MS: m/z: 362.2 [M+H]⁺

### Synthetic Example 7: Synthesis of 2-((6-methylimidazo[1,2-a]pyridin-2-yl)methyl)-5-(3-phenylazetidin-1-yl) 2,7-naphthyridin-1(2H)-one. (Compound No. 7)

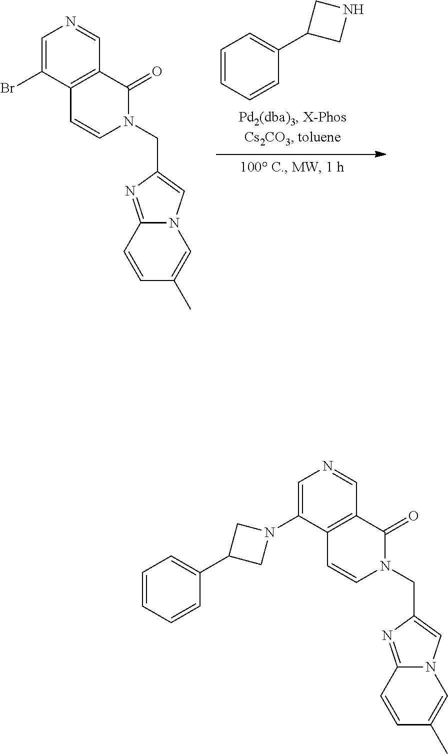 [SMILES: Cc1ccc2nc(Cn3ccc4c(Br)cncc4c3=O)cn2c1 ; c1ccc(C2CNC2)cc1 ; Cc1ccc2nc(Cn3ccc4c(N5CC(c6ccccc6)C5)cncc4c3=O)cn2c1]

The title compound was prepared using the procedure described for example 5, step 1 with the following modification: the reaction was performed with 3-phenylazetidine to give 2-({6-methylimidazo[1,2-a]pyridin-2-yl}methyl)-5-(3-phenylazetidin-1-yl)-1,2-dihydro-2,7-naphthyridin-1-one (yield: 21%) as an off white solid. ¹H NMR (400 MHz, DMSO-d₆): δ 8.84 (s, 1H), 8.29 (s, 1H), 7.98 (s, 1H), 7.78 (s, 1H), 7.70 (d, J=7.6 Hz, 1H), 7.45-7.34 ((m, 5H), 7.26 (t, J=6.8 Hz, 1H), 7.08 (d, J=9.2 Hz, 1H), 6.66 (d, J=7.6 Hz, 1H), 5.25 (s, 2H), 4.57 (t, J=7.6 Hz, 2H), 4.13 (t, J=6.4 Hz, 2H), 4.04-4.01 (m, 1H), 2.24 (s, 3H). MS: m/z: 422 [M+H]⁺

### Synthetic Example 8: Synthesis of 5-(4-hydroxypiperidin-1-yl)-2-((6-methylimidazo[1,2-a]pyridin-2-yl)methyl)-2,7-naphthyridin-1(2H)-one. (Compound No. 8)

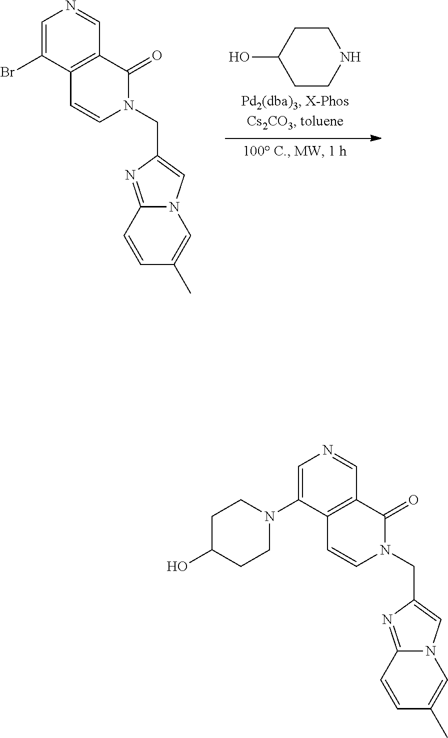 [SMILES: Cc1ccc2nc(Cn3ccc4c(Br)cncc4c3=O)cn2c1 ; OC1CCNCC1 ; Cc1ccc2nc(Cn3ccc4c(N5CCC(O)CC5)cncc4c3=O)cn2c1]

The title compound was prepared using the procedure described for example 5, step 1 with the following modification: the reaction was performed with 4-hydroxy piperidine to give 5-(4-hydroxypiperidin-1-yl)-2-({6-methylimidazo[1,2-a]pyridin-2-yl}methyl)-1,2-dihydro-2,7-naphthyridin-1-one (yield: 20%) as an off-white solid. ¹H NMR (400 MHz, DMSO-d₆): δ 9.04 (s, 1H), 8.39 (s, 1H), 8.35 (s, 1H), 7.85 (d, J=4.4 Hz, 1H), 7.82 (s, 1H), 7.47 (d, J=9.2 Hz, 1H), 7.21 (d, J=9.2 Hz, 1H), 6.63 (d, J=7.6 Hz, 1H), 5.30 (s, 2H), 3.68-3.66 (m, 1H), 3.28-3.19 (m, 2H), 2.84 (t, J=5.6 Hz, 2H), 2.27 (s, 3H), 1.96-1.86 ((m, 2H), 1.68-1.57 ((m, 2H). MS: m/z: 390 [M+H]⁺

### Synthetic Example 9: Synthesis of 5-(azetidin-1-yl)-2-((6-methylimidazo[1,2-a]pyridin-2-yl)methyl)-2,7-naphthyridin-1(2H)-one. (Compound No. 9)

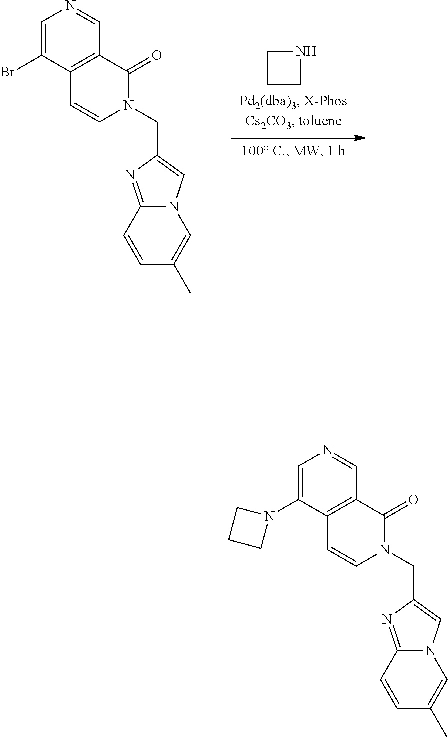

The title compound was prepared using the procedure described for example 5, step 1 with the following modification: the reaction was performed with azetidine to give 5-(azetidin-1-yl)-2-({6-methylimidazo[1,2-a]pyridin-2-yl}methyl)-1,2-dihydro-2,7-naphthyridin-1-one (yield: 34%) as an off white solid. ¹H NMR (400 MHz, DMSO-d6) δ 8.79 (s, 1H), 8.29 (s, 1H), 7.87 (s, 1H), 7.77 (s, 1H), 7.67 (d, J=7.6 Hz, 1H), 7.40 (d, J=9.2 Hz, 1H), 7.08 (dd, J=9.2, 1.6 Hz, 1H), 6.58 (d, J=8.0 Hz, 1H), 5.24 (s, 2H), 4.12 (t, J=7.2 Hz, 4H), 2.36-2.32 (m, 2H), 2.24 (s, 3H).

MS: m/z: 346 [M+H]⁺

### Synthetic Example 10: 2-((6-methylimidazo[1,2-a]pyridin-2-yl)methyl)-5-(piperidin-1-yl)-2,7-naphthyridin-1(2H)-one. (Compound No. 10)

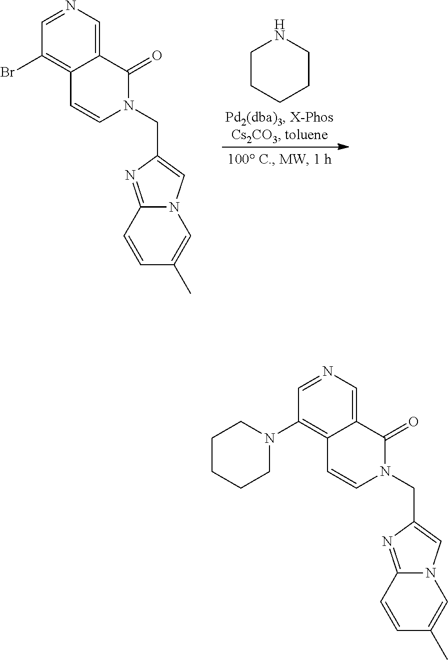 [SMILES: Cc1ccc2nc(Cn3ccc4c(Br)cncc4c3=O)cn2c1 ; C1CCNCC1 ; Cc1ccc2nc(Cn3ccc4c(N5CCCCC5)cncc4c3=O)cn2c1]

The title compound was prepared using the procedure described for example 5, step 1 with the following modification: the reaction was performed with piperidine to give 2-({6-methylimidazo[1,2-a]pyridin-2-yl}methyl)-5-(piperidin-1-yl)-1,2-dihydro-2,7-naphthyridin-1-one (yield: 17%) as a brown solid. ¹H NMR (400 MHz, DMSO-d₆): δ 9.04 (s, 1H), 8.37 (s, 1H), 8.28 (s, 1H), 7.82 (d, J=7.6 Hz, 1H), 7.76 (s, 1H), 7.39 (d, J=9.2 Hz, 1H), 7.08 (dd, J=9.2, 1.6 Hz, 1H), 6.62 (d, J=7.6 Hz, 1H), 5.26 (s, 2H), 2.99 (t, J=5.2 Hz, 4H), 2.24 (s, 3H), 1.72 (t, J=5.2 Hz, 4H), 1.58 (d, J=4.8 Hz, 2H). MS: m/z: 374 [M+H]⁺

### Synthetic Example 11: Synthesis of 5-cyclopropyl-2-((6-methylimidazo[1,2-a]pyridin-2-yl)methyl)-2,7-naphthyridin-1(2H)-one. (Compound No. 11)

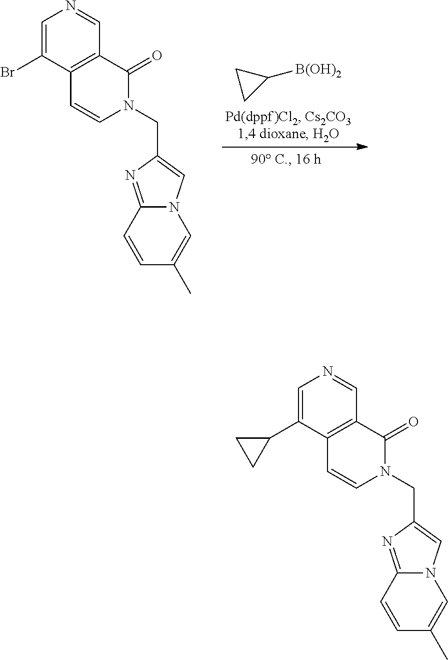 [SMILES: Cc1ccc2nc(Cn3ccc4c(Br)cncc4c3=O)cn2c1 ; OB(O)C1CC1 ; Cc1ccc2nc(Cn3ccc4c(C5CC5)cncc4c3=O)cn2c1]

A stirred solution of 5-bromo-2-({6-methylimidazo[1,2-a]pyridin-2-yl}methyl)-1,2-dihydro-2,7-naphthyridin-1-one(70 mg, 190 μmol) in mixture of 1,4-dioxane & water (4:1) (2.5 mL) was purged with argon for 20 min, followed by addition of cyclopropylboronic acid (19.5 mg, 228 μmol), Cs₂CO₃ (185 mg, 569 μmol) and Pd(dppf)Cl₂-DCM complex (20.0 mg, 24.5 μmol) and heated at 90° C. for 16 h. The reaction mixture was cooled to room temperature, filtered through a celite bed, rinsed with ethyl acetate (20 mL), and the filtrate was concentrated in vacuo and purified by reverse phase Prep HPLC (Column: YMC Triart C18 (250*20 mm)₅μ 0-95% ACN/H₂O) to give rise to 5-cyclopropyl-2-({6-methylimidazo[1,2-a]pyridin-2-yl}methyl)-1,2-dihydro-2,7-naphthyridin-1-one (12.0 mg, yield: 19%) as an off-white solid. ¹H NMR (400 MHz, DMSO-d₆): δ 9.21 (s, 1H), 8.44 (s, 1H), 8.29 (s, 1H), 7.92 (d, J=7.6 Hz, 1H), 7.78 (s, 1H), 7.40 (d, J=9.3 Hz, 1H), 7.08 (dd, J=9.2, 1.6 Hz, 1H), 6.96 (d, J=7.6 Hz, 1H), 5.28 (s, 2H), 2.24 (s, 3H), 2.15-2.10 (m, 1H), 1.04-1.01 (m, 2H), 0.80-0.76 (m, 2H). MS: m/z: 331 [M+H]⁺

### Synthetic Example 12: Synthesis of 2-((6-methylimidazo[1,2-a]pyridin-2-yl)methyl)-5-(pyridin-4-yl)-2,7-naphthyridin-1(2H)-one. (Compound No. 12)

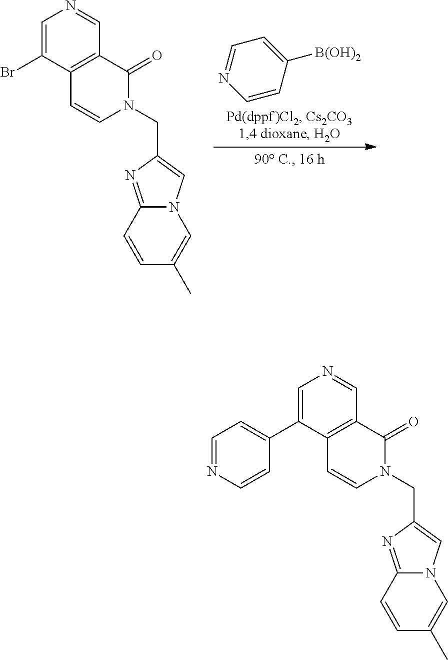 [SMILES: Cc1ccc2nc(Cn3ccc4c(Br)cncc4c3=O)cn2c1 ; OB(O)c1ccncc1 ; Cc1ccc2nc(Cn3ccc4c(-c5ccncc5)cncc4c3=O)cn2c1]

The title compound was prepared using the procedure described for example 11 with the following modification: the reaction was performed with 4-pyridineboronic acid to give 2-({6-methylimidazo[1,2-a]pyridin-2-yl}methyl)-5-(pyridin-4-yl)-1,2-dihydro-2,7-naphthyridin-1-one (yield: 17%) as an off-white solid. ¹H NMR (400 MHz, DMSO-d₆): δ 9.44 (s, 1H), 8.74 (dd, J=4.4, 0.4 Hz, 2H), 8.72 (s, 1H), 8.29 (s, 1H), 7.89 (d, J=7.6 Hz, 1H), 7.81 (s, 1H), 7.56 (dd, J=4.4, 0.4 Hz, 2H), 7.39 (d, J=9.2 Hz, 1H), 7.08 (dd, J=9.2, 1.6 Hz, 1H), 6.48 (d, J=7.6 Hz, 1H), 5.30 (s, 2H), 2.24 (s, 3H). MS: m/z: 368 [M+H]⁺

### Synthetic Example 13: 2-((6-methylimidazo[1,2-a]pyridin-2-yl)methyl)-5-phenyl-2,7-naphthyridin-1(2H)-one. (Compound No. 13)

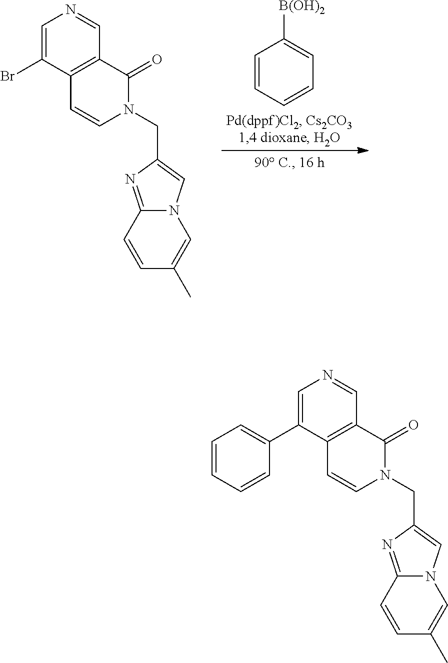 [SMILES: Cc1ccc2nc(Cn3ccc4c(Br)cncc4c3=O)cn2c1 ; OB(O)c1ccccc1 ; Cc1ccc2nc(Cn3ccc4c(-c5ccccc5)cncc4c3=O)cn2c1]

The title compound was prepared using the procedure described for example 11 with the following modification: the reaction was performed with phenyl boronic acid to give 2-({6-methylimidazo[1,2-a]pyridin-2-yl}methyl)-5-phenyl-1,2-dihydro-2,7-naphthyridin-1-one (yield: 43%) as a pale brown solid. ¹H NMR (400 MHz, DMSO-d₆) δ 9.39 (d, J=0.4 Hz, 1H), 8.66 (s, 1H), 8.29 (s, 1H), 7.85 (d, J=7.6 Hz, 1H), 7.80 (s, 1H), 7.60-7.46 (m, 5H), 7.39 (d, J=9.2 Hz, 1H), 7.08 (dd, J=9.2, 1.6 Hz, 1H), 6.47 (d, J=7.6 Hz, 1H), 5.28 (s, 2H), 2.24 (s, 3H). MS: m/z: 367.2 [M+H]⁺

### Synthetic Example 14: 2-((6-methylimidazo[1,2-a]pyridin-2-yl)methyl)-5-(1H-pyrazol-4-yl)-2,7-naphthyridin-1(2H)-one. (Compound No. 14)

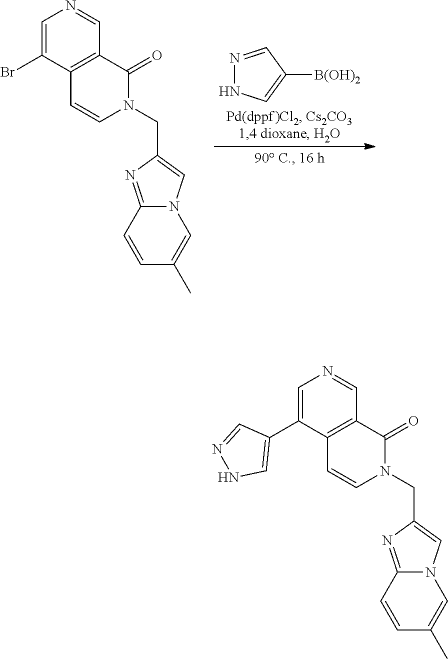 [SMILES: Cc1ccc2nc(Cn3ccc4c(Br)cncc4c3=O)cn2c1 ; OB(O)c1cn[nH]c1 ; Cc1ccc2nc(Cn3ccc4c(-c5cn[nH]c5)cncc4c3=O)cn2c1]

The title compound was prepared using the procedure described for example 11 with the following modification: the reaction was performed with pyrazole-4-boronic acid to give 2-({6-methylimidazo[1,2-a]pyridin-2-yl}methyl)-5-(1H-pyrazol-4-yl)-1,2-dihydro-2,7-naphthyridin-1-one (yield: 37%) as an off-white solid. ¹H NMR (400 MHz, DMSO-d₆): δ 13.26 (bs, 1H), 9.28 (s, 1H), 8.75 (s, 1H), 8.29 (s, 1H), 8.19 (s, 1H), 7.85 (d, J=7.6 Hz, 2H), 7.80 (s, 1H), 7.40 (d, J=9.2 Hz, 1H), 7.08 (dd, J=9.2, 1.6 Hz, 1H), 6.79 (d, J=7.6 Hz, 1H), 5.29 (s, 2H), 2.24 (s, 3H).

MS: m/z: 357 [M+H]⁺

### Synthetic Example 15: Synthesis of 2-((6-methylimidazo[1,2-a]pyridin-2-yl)methyl)-5-(1H-pyrazol-3-yl)-2,7-naphthyridin-1(2H)-one. (Compound No. 15)

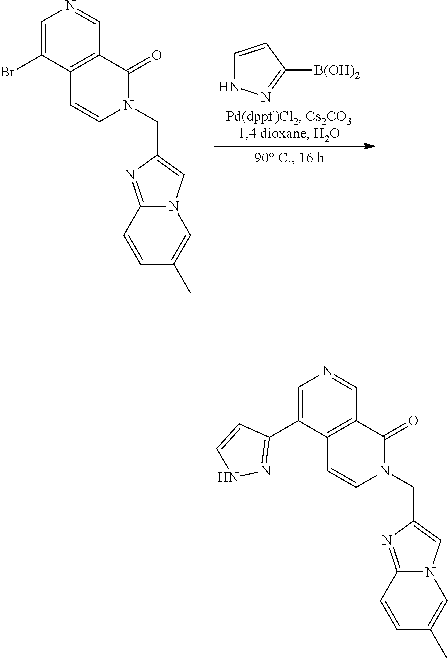 [SMILES: Cc1ccc2nc(Cn3ccc4c(Br)cncc4c3=O)cn2c1 ; OB(O)c1cc[nH]n1 ; Cc1ccc2nc(Cn3ccc4c(-c5cc[nH]n5)cncc4c3=O)cn2c1]

The title compound was prepared using the procedure described for example 11 with the following modification: the reaction was performed with pyrazole-3-boronic acid to afford 2-({6-methylimidazo[1,2-a]pyridin-2-yl}methyl)-5-(1H-pyrazol-4-yl)-1,2-dihydro-2,7-naphthyridin-1-one (yield: 13%) as an off-white solid. ¹H NMR (400 MHz, DMSO-d₆): δ 13.25 (s, 1H), 9.34 (s, 1H), 8.97 (s, 1H), 8.31 (s, 1H), 7.93 (s, 1H), 7.88 (d, J=7.6 Hz, 1H), 7.81 (s, 1H), 7.47 (bs, 1H), 7.42 (d, J=9.2 Hz, 1H), 7.12 (dd, J=9.2, 1.6 Hz, 1H), 6.81 (s, 1H), 5.30 (s, 2H), 2.25 (s, 3H). MS: m/z: 357 [M+H]⁺

### Synthetic Example 16: Synthesis of 2-((6-methylimidazo[1,2-a]pyridin-2-yl)methyl)-5-(pyridin-2-yl)-2,7-naphthyridin-1(2H)-one. (Compound No. 16)

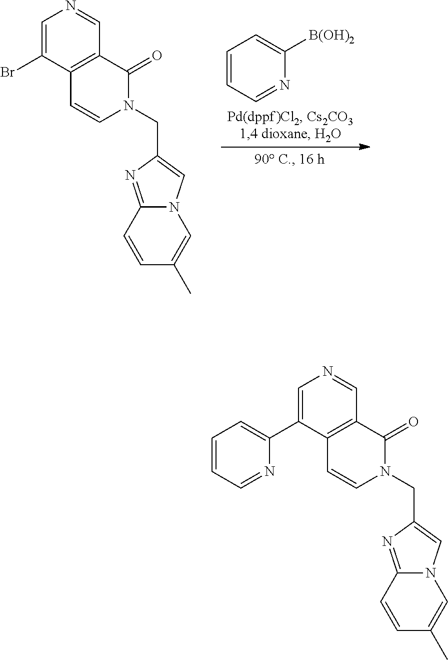 [SMILES: Cc1ccc2nc(Cn3ccc4c(Br)cncc4c3=O)cn2c1 ; OB(O)c1ccccn1 ; Cc1ccc2nc(Cn3ccc4c(-c5ccccn5)cncc4c3=O)cn2c1]

The title compound was prepared using the procedure described for example 11 with the following modification: the reaction was performed with pyridine-2-boronic acid to give rise to 2-({6-methylimidazo[1,2-a]pyridin-2-yl}methyl)-5-(pyridin-2-yl)-1,2-dihydro-2,7-naphthyridin-1-one (yield: 40%) as an off-white solid. ¹H NMR (400 MHz, DMSO-d₆): δ 9.44 (d, J=0.8 Hz, 1H), 8.85 (s, 1H), 8.77 (ddd, J=4.8, 1.6, 0.8 Hz, 1H), 8.29 (t, J=0.8 Hz, 1H), 8.00 (td, J=7.6, 1.6 Hz, 1H), 7.86 (d, J=7.6 Hz, 1H), 7.78-7.70 (m, 2H), 7.51 (ddd, J=7.6, 4.8, 1.2 Hz, 1H), 7.39 (d, J=9.2 Hz, 1H), 7.08 (dd, J=9.2, 1.6 Hz, 1H), 6.86 (dd, J=7.6, 0.8 Hz, 1H), 5.29 (s, 2H), 2.24 (d, J=0.8 Hz, 3H). MS: m/z: 368 [M+H]⁺

### Synthetic Example 17: Synthesis of 2-((6-methylimidazo[1,2-a]pyridin-2-yl)methyl)-5-(pyridin-3-yl)-2,7-naphthyridin-1(2H)-one. (Compound No. 17)

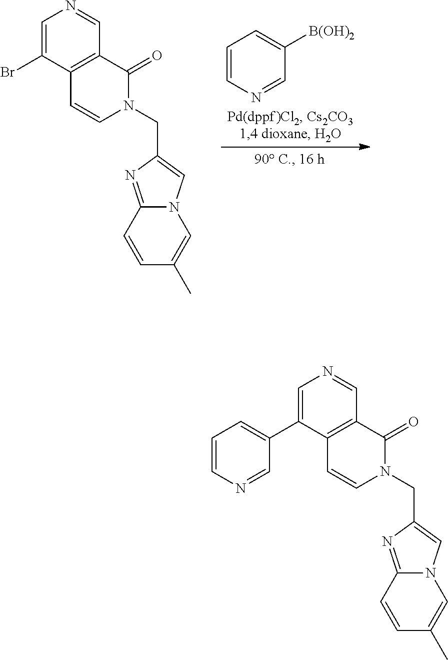 [SMILES: Cc1ccc2nc(Cn3ccc4c(Br)cncc4c3=O)cn2c1 ; OB(O)c1cccnc1 ; Cc1ccc2nc(Cn3ccc4c(-c5cccnc5)cncc4c3=O)cn2c1]

The title compound was prepared using the procedure described for example 11 with the following modification: the reaction was performed with pyridine-3-boronic acid to give 2-({6-methylimidazo[1,2-a]pyridin-2-yl}methyl)-5-(pyridin-3-yl)-1,2-dihydro-2,7-naphthyridin-1-one (yield: 40%) as an off-white solid. ¹H NMR (400 MHz, DMSO-d₆): δ 9.43 (s, 1H), 8.72-8.70 (m, 3H), 8.29 (s, 1H), 7.96 (dt, J=7.6, 1.6 Hz, 1H), 7.88 (d, J=7.6 Hz, 1H), 7.81 (s, 1H), 7.59 (dd, J=7.6, 4.8 Hz, 1H), 7.39 (d, J=9.2 Hz, 1H), 7.08 (dd, J=9.2, 1.6 Hz, 1H), 6.43 (d, J=7.6 Hz, 1H), 5.29 (s, 2H), 2.24 (s, 3H). MS: m/z: 368 [M+H]⁺

### Synthetic Example 18: Synthesis of 5-(4-methoxyphenyl)-2-((6-methylimidazo[1,2-a]pyridin-2-yl)methyl)-2,7-naphthyridin-1(2H)-one. (Compound No. 18)

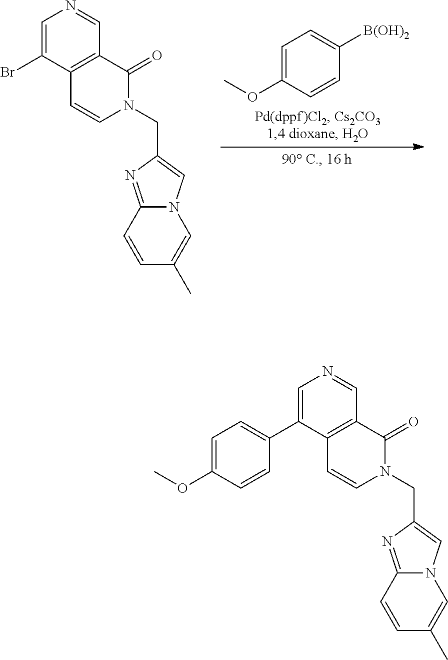

The title compound was prepared using the procedure described for example 11 with the following modification: the reaction was performed with 4-methoxyphenyl boronic acid to give 5-(4-methoxyphenyl)-2-({6-methylimidazo[1,2-a]pyridin-2-yl}methyl)-1,2-dihydro-2,7-naphthyridin-1-one (yield: 22%) as an off-white solid. ¹H NMR (400 MHz, DMSO-d₆): δ 9.36 (s, 1H), 8.63 (s, 1H), 8.29 (s, 1H), 7.84 (d, J=7.6 Hz, 1H), 7.79 (s, 1H), 7.43-7.38 (m, 3H), 7.11-7.07 (m, 3H), 6.49 (d, J=7.6 Hz, 1H), 5.28 (s, 2H), 3.83 (s, 3H), 2.24 (s, 3H). MS: m/z: 397 [M+H]⁺

### Synthetic Example 19: Synthesis of 5-(3-methoxyphenyl)-2-((6-methylimidazo[1,2-a]pyridin-2-yl)methyl)-2,7-naphthyridin-1(2H)-one. (Compound No. 19)

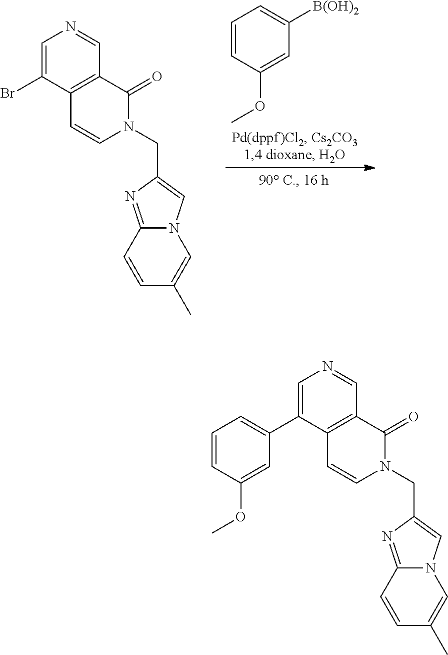 [SMILES: Cc1ccc2nc(Cn3ccc4c(Br)cncc4c3=O)cn2c1 ; COc1cccc(B(O)O)c1 ; COc1cccc(-c2cncc3c(=O)n(Cc4cn5cc(C)ccc5n4)ccc23)c1]

The title compound was prepared using the procedure described for example 11 with the following modification: the reaction was performed with 3-methoxyphenyl boronic acid to give 5-(3-methoxyphenyl)-2-({6-methylimidazo[1,2-a]pyridin-2-yl}methyl)-1,2-dihydro-2,7-naphthyridin-1-one (yield: 26%) as an off-white solid. ¹H NMR (400 MHz, DMSO-d₆): δ 9.39 (s, 1H), 8.67 (s, 1H), 8.29 (s, 1H), 7.84 (d, J=7.6 Hz, 1H), 7.80 (s, 1H), 7.46 (t, J=8.0 Hz, 1H), 7.39 (d, J=9.2 Hz, 1H), 7.10-7.03 (m, 4H), 6.50 (d, J=7.6 Hz, 1H), 5.28 (s, 2H), 3.82 (s, 3H), 2.24 (s, 3H). MS: m/z: 397 [M+H]⁺

### Synthetic Example 20: Synthesis of 5-(1-methyl-1H-pyrazol-3-yl)-2-((6-methylimidazo[1,2-a]pyridin-2-yl)methyl)-2,7-naphthyridin-1(2H)-one. (Compound No. 20)

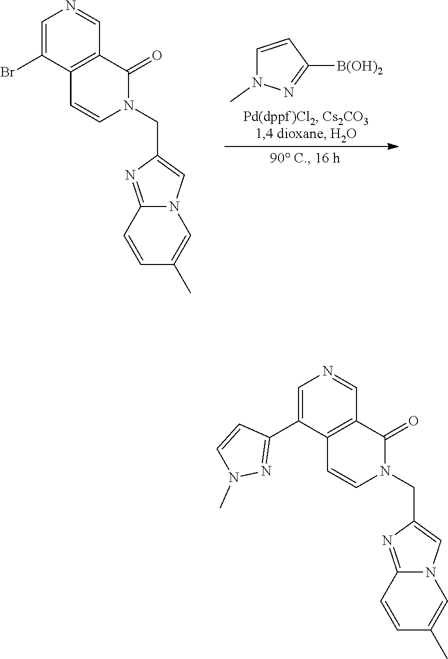

The title compound was prepared using the procedure described for example 11 with the following modification: the reaction was performed with 1-methylpyrazole-3-boronic acid to afford 5-(1-methyl-1H-pyrazol-3-yl)-2-({6-methylimidazo[1,2-a]pyridin-2-yl}methyl)-1,2-dihydro-2,7-naphthyridin-1-one (yield: 24%) as an off-white solid. ¹H NMR (400 MHz, DMSO-d₆): δ 9.32 (d, J=0.4 Hz, 1H), 8.95 (s, 1H), 8.28 (t, J=0.8 Hz, 1H), 7.88 (dd, J=5.2, 2.8 Hz, 2H), 7.78 (s, 1H), 7.45-7.38 (m, 2H), 7.08 (dd, J=9.2, 1.6 Hz, 1H), 6.78 (d, J=2.0 Hz, 1H), 5.29 (s, 2H), 3.96 (s, 3H), 2.24 (d, J=0.8 Hz, 3H). MS: m/z: 371 [M+H]⁺

### Synthetic Example 21: Synthesis of 2-((6-methylimidazo[1,2-a]pyridin-2-yl)methyl)-5-(o-tolyl)-2,7-naphthyridin-1(2H)-one. (Compound No. 21)

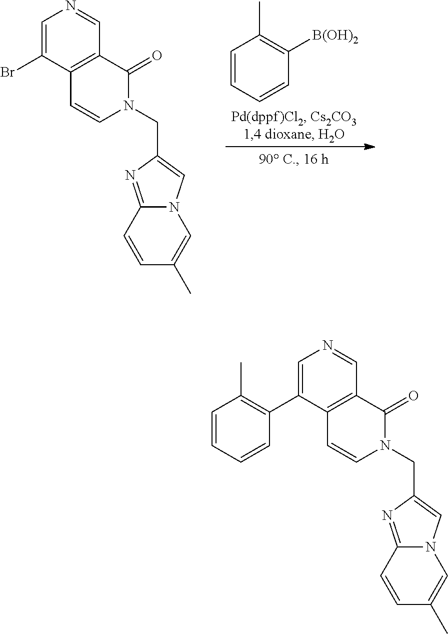

The title compound was prepared using the procedure described for example 11 with the following modification: the reaction was performed with o-tolylboronic acid to give 2-({6-methylimidazo[1,2-a]pyridin-2-yl}methyl)-5-(2-methylphenyl)-1,2-dihydro-2,7-naphthyridin-1-one (yield: 33%) as an off-white solid. ¹H NMR (400 MHz, DMSO-d₆): δ 9.40 (d, J=0.8 Hz, 1H), 8.56 (s, 1H), 8.30 (t, J=0.8 Hz, 1H), 7.81-7.78 (m, 2H), 7.41-7.38 (m, 3H), 7.35-7.31 (m, 1H), 7.21 (d, J=7.2 Hz, 1H), 7.08 (dd, J=9.2, 1.6 Hz, 1H), 5.99 (dd, 0.8 Hz, 1H), 5.30 (d, J=14.4 Hz, 1H), 5.23 (d, J=14.8 Hz, 1H), 2.24 (s, 3H), 2.02 (s, 3H). MS: m/z: 381 [M+H]⁺

### Synthetic Example 22: Synthesis of 5-(4-fluorophenyl)-2-((6-methylimidazo[1,2-a]pyridin-2-yl)methyl)-2,7-naphthyridin-1(2H)-one. (Compound No. 22)

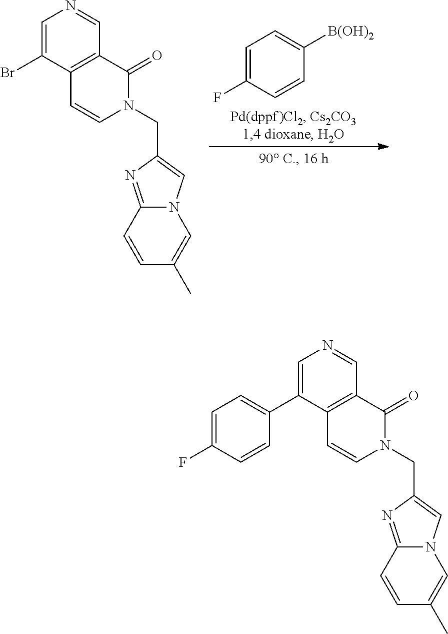 [SMILES: Cc1ccc2nc(Cn3ccc4c(Br)cncc4c3=O)cn2c1 ; OB(O)c1ccc(F)cc1 ; Cc1ccc2nc(Cn3ccc4c(-c5ccc(F)cc5)cncc4c3=O)cn2c1]

The title compound was prepared using the procedure described for example 11 with the following modification: the reaction was performed with 4-fluorophenyl boronic acid to give 5-(4-fluorophenyl)-2-({6-methylimidazo[1,2-a]pyridin-2-yl}methyl)-1,2-dihydro-2,7-naphthyridin-1-one (yield: 52%) as an off-white solid. ¹H NMR (400 MHz, DMSO-d₆): δ 9.43 (s, 1H), 8.72 (s, 1H), 8.65 (s, 1H), 8.24 (s, 1H), 7.90 (d, J=7.6 Hz, 1H), 7.82 (d, J=9.2 Hz, 1H), 7.76 (dd, J=9.2, 1.6 Hz, 1H), 7.56-7.51 (m, 2H), 7.44-7.37 (m, 2H), 6.52 (d, J=7.6 Hz, 1H), 5.47 (s, 2H), 2.38 (s, 3H). MS: m/z: 385 [M+H]⁺

### Synthetic Example 23: Synthesis of 2-((6-methylimidazo[1,2-a]pyridin-2-yl)methyl)-5-(morpholinomethyl)-2,7-naphthyridin-1(2H)-one. (Compound No. 23)

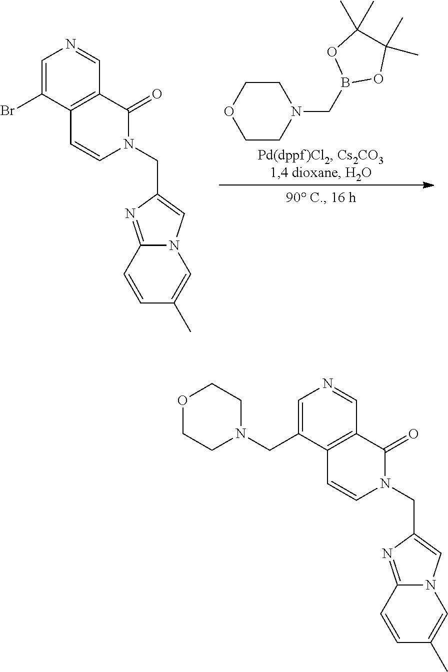 [SMILES: Cc1ccc2nc(Cn3ccc4c(Br)cncc4c3=O)cn2c1 ; CC1(C)OB(CN2CCOCC2)OC1(C)C ; Cc1ccc2nc(Cn3ccc4c(CN5CCOCC5)cncc4c3=O)cn2c1]

The title compound was prepared using the procedure described for example 11 with the following modification: the reaction was performed with 4-morpholinomethyl boronic acid to give 2-({6-methylimidazo[1,2-a]pyridin-2-yl}methyl)-5-[(morpholin-4-yl)methyl]-1,2-dihydro-2,7-naphthyridin-1-one (yield: 24%) as an off-white solid. ¹H NMR (400 MHz, DMSO-d₆): δ 9.29 (s, 1H), 8.59 (s, 1H), 8.33-8.27 (m, 1H), 7.91-7.82 (m, 1H), 7.80 (s, 1H), 7.40 (d, J=9.2 Hz, 1H), 7.08 (dd, J=9.2, 1.6 Hz, 1H), 6.89 (d, J=7.6 Hz, 1H), 5.27 (s, 2H), 3.68 (s, 2H), 3.53 (t, J=4.4 Hz, 4H), 2.40 (t, J=4.4 Hz, 4H), 2.24 (s, 3H). MS: m/z: 390 [M+H]⁺

### Synthetic Example 24: Synthesis of 5-(2-fluorophenyl)-2-((6-methylimidazo[1,2-a]pyridin-2-yl)methyl)-2,7-naphthyridin-1(2H)-one. (Compound No. 24)

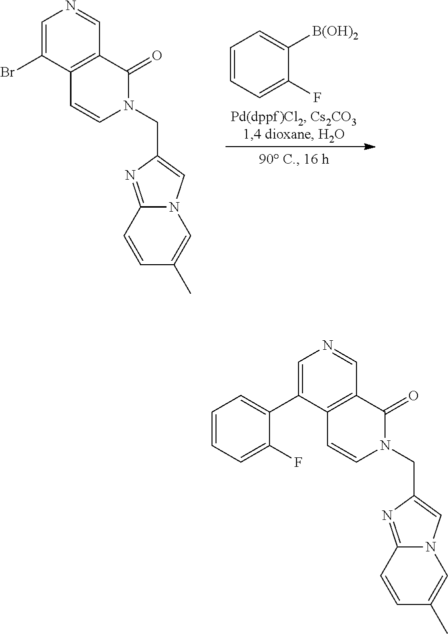 [SMILES: Cc1ccc2nc(Cn3ccc4c(Br)cncc4c3=O)cn2c1 ; OB(O)c1ccccc1F ; Cc1ccc2nc(Cn3ccc4c(-c5ccccc5F)cncc4c3=O)cn2c1]

The title compound was prepared using the procedure described for example 11 with the following modification: the reaction was performed with 2-fluorophenyl boronic acid to give 5-(3-fluorophenyl)-2-({6-methylimidazo[1,2-a]pyridin-2-yl}methyl)-1,2-dihydro-2,7-naphthyridin-1-one (33%) as an off white solid. ¹H NMR (400 MHz, DMSO-d₆): δ 9.41 (s, 1H), 8.69 (s, 1H), 8.29 (s, 1H), 7.86 (d, J=7.6 Hz, 1H), 7.81 (s, 1H), 7.60 (s, 1H), 7.41-7.35 (m, 4H), 7.09 (d, J=8.4 Hz, 1H), 6.47 (d, J=6.4 Hz, 1H), 5.29 (s, 2H), 2.24 (s, 3H). MS: m/z: 385 [M+H]⁺

### Synthetic Example 25: Synthesis of 5-(2,3-difluorophenyl)-2-((6-methylimidazo[1,2-a]pyridin-2-yl)methyl)-2,7-naphthyridin-1(2H)-one. (Compound No. 25)

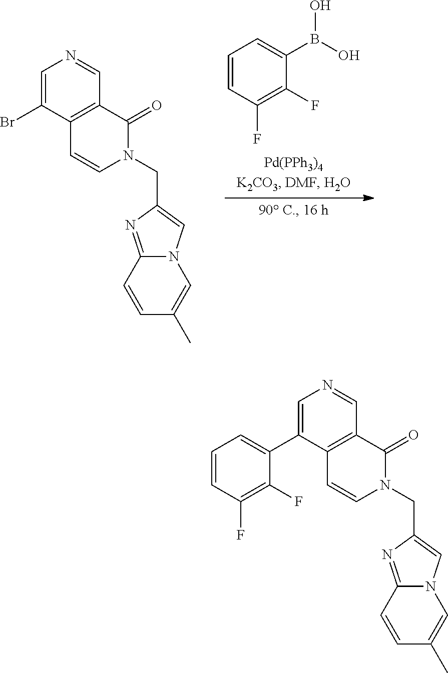 [SMILES: Cc1ccc2nc(Cn3ccc4c(Br)cncc4c3=O)cn2c1 ; OB(O)c1cccc(F)c1F ; Cc1ccc2nc(Cn3ccc4c(-c5cccc(F)c5F)cncc4c3=O)cn2c1]

The title compound was prepared using the procedure described for example 11 with the following modification: the reaction was performed with 2,3-difluorophenyl boronic acid to give 5-(2,3-difluorophenyl)-2-({6-methylimidazo[1,2-a]pyridin-2-yl}methyl)-1,2-dihydro-2,7-naphthyridin-1-one (yield: 35%) as an off-white solid. ¹H NMR (400 MHz, DMSO-d₆): δ 9.44 (d, J=0.7 Hz, 1H), 8.72 (s, 1H), 8.29 (d, J=0.4 Hz, 1H), 7.87 (d, J=7.2 Hz, 1H), 7.81 (s, 1H), 7.62-7.60 (m, 1H), 7.40-7.37 (m, 2H), 7.33-7.30 (m, 1H), 7.08 (dd, J=9.2, 1.6 Hz, 1H), 6.28 (dd, J=7.6, 1.6 Hz, 1H), 5.29 (s, 2H), 2.24 (s, 3H). MS: m/z: 403[M+H]⁺

### Synthetic Example 26: Synthesis of N-methyl-4-(7-((6-methylimidazo[1,2-a]pyridin-2-yl)methyl)-8-oxo-7,8-dihydro-2,7-naphthyridin-4-yl)benzamide. (Compound No. 26)

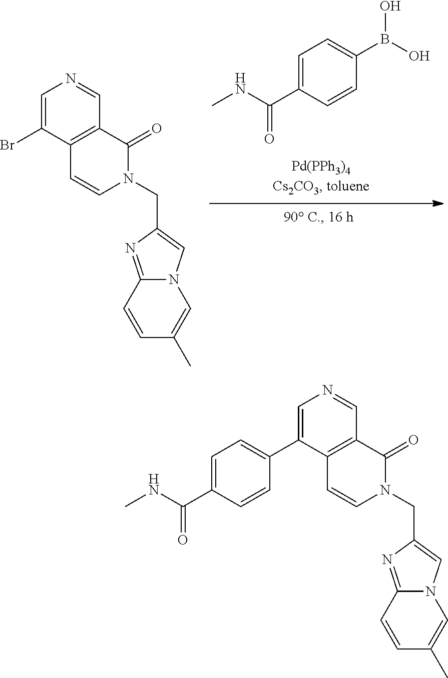

The title compound was prepared using the procedure described for example 11 with the following modification: the reaction was performed with 4-acetamidophenyl boronic acid to give N-methyl-4-[7-({6-methylimidazo[1,2-a]pyridin-2-yl}methyl)-8-oxo-7,8-dihydro-2,7-naphthyridin-4-yl]benzamide (yield: 19%) as an white solid. ¹H NMR (400 MHz, DMSO-d₆): δ 9.41 (s, 1H), 8.69 (s, 1H), 8.57 (d, J=4.4 Hz, 1H), 8.29 (s, 1H), 8.00 (d, J=8.4 Hz, 2H), 7.86 (d, J=7.6 Hz, 1H), 7.80 (s, 1H), 7.30 (d, J=8.4 Hz, 2H), 7.39 (d, J=9.2 Hz, 1H), 7.08 (dd, J=9.2, 1.2 Hz, 1H), 6.46 (d, J=7.6 Hz, 1H), 5.29 (s, 2H), 2.83 (d, J=4.8 Hz, 3H), 2.24 (s, 3H). MS: m/z: 424 [M+H]⁺

### Synthetic Example 27: Synthesis of 2-((6-methylimidazo[1,2-a]pyridin-2-yl)methyl)-5-(4-(morpholine-4-carbonyl) phenyl)-2,7-naphthyridin-1(2H)-one. (Compound No. 27)

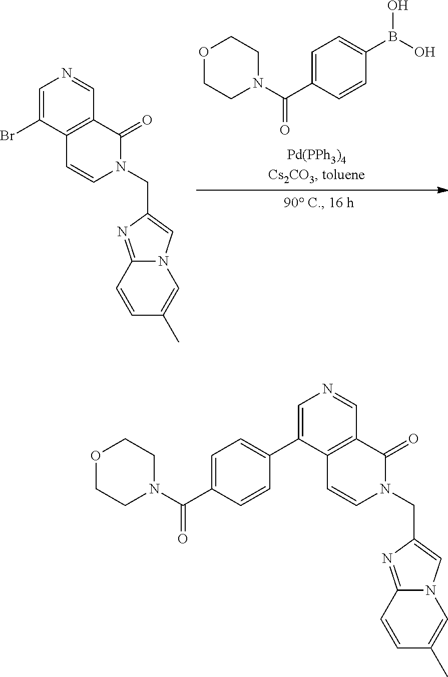 [SMILES: Cc1ccc2nc(Cn3ccc4c(Br)cncc4c3=O)cn2c1 ; O=C(c1ccc(B(O)O)cc1)N1CCOCC1 ; Cc1ccc2nc(Cn3ccc4c(-c5ccc(C(=O)N6CCOCC6)cc5)cncc4c3=O)cn2c1]

The title compound was prepared using the procedure described for example 11 with the following modification: the reaction was performed with 4-(morpoline-4-carbonyl)phenyl boronic acid to give 2-({6-methylimidazo[1,2-a]pyridin-2-yl}methyl)-5-[4-(morpholine-4-carbonyl)phenyl]-1,2-dihydro-2,7-naphthyridin-1-one(yield: 74%) as a colourless gummy liquid. ¹H NMR (400 MHz, DMSO-d₆): δ 9.45 (s, 1H), 8.76 (s, 1H), 8.68 (s, 1H), 8.27 (s, 1H), 7.92 (d, J=7.6 Hz, 1H), 7.85 (d, J=9.2 Hz, 1H), 7.80 (dd, J=9.2, 1.6 Hz, 1H), 7.59 (q, J=7.6 Hz, 4H), 6.58 (d, J=7.6 Hz, 1H), 5.49 (s, 2H), 3.71-3.52 ((m, 4H), 3.49-3.36 (m, 4H), 2.39 (s, 3H). MS: m/z: 480 [M+H]⁺

### Synthetic Example 28: Synthesis of 2-(7-((6-methylimidazo[1,2-a]pyridin-2-yl)methyl)-8-oxo-7,8-dihydro-2,7-naphthyridin-4-yl)benzonitrile.(Compound No. 28)

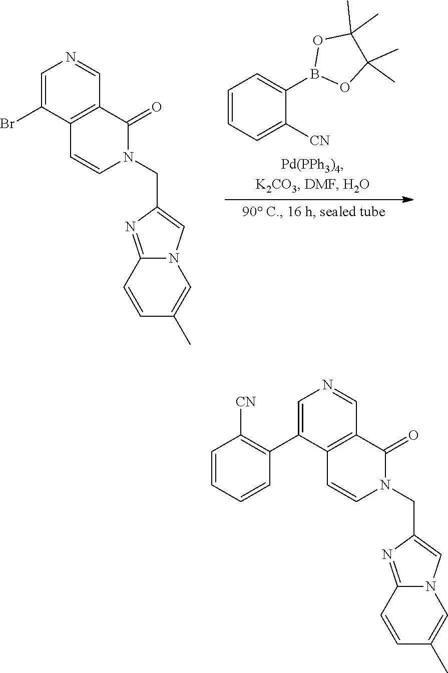 [SMILES: Cc1ccc2nc(Cn3ccc4c(Br)cncc4c3=O)cn2c1 ; CC1(C)OB(c2ccccc2C#N)OC1(C)C ; Cc1ccc2nc(Cn3ccc4c(-c5ccccc5C#N)cncc4c3=O)cn2c1]

The title compound was prepared using the procedure described for example 11 with the following modification: the reaction was performed with 2-cyanophenyl boronic acid pinacol ester to give 2-[7-({6-methylimidazo[1,2-a]pyridin-2-yl}methyl)-8-oxo-7,8-dihydro-2,7-naphthyridin-4-yl]benzonitrile (yield: 14%) as an off-white solid. ¹H NMR (400 MHz, DMSO-d₆): δ 9.47 (d, J=0.1 Hz, 1H), 8.74 (s, 1H), 8.30 (s, 1H), 8.07 (dd, J=7.6, 1.2 Hz, 1H), 7.90-7.88 (m, 2H), 7.82 (s, 1H), 7.73 (dt, J=7.6, 1.2 Hz, 1H), 7.65 (dd, J=7.8, 1.2 Hz, 1H), 7.40 (d, J=9.2 Hz, 1H), 7.10 (dd, J=9.2, 1.8 Hz, 1H), 6.16 (d, J=7.6 Hz, 1H), 5.34 (d, J=14.8 Hz, 1H), 5.25 (d, J=14.8 Hz, 1H), 2.24 (s, 3H); MS: m/z: 392 [M+H]⁺

### Synthetic Example 29: Synthesis of 5-(2,6-difluorophenyl)-2-((6-methylimidazo[1,2-a]pyridin-2-yl)methyl)-2,7-naphthyridin-1(2H)-one. (Compound No. 29)

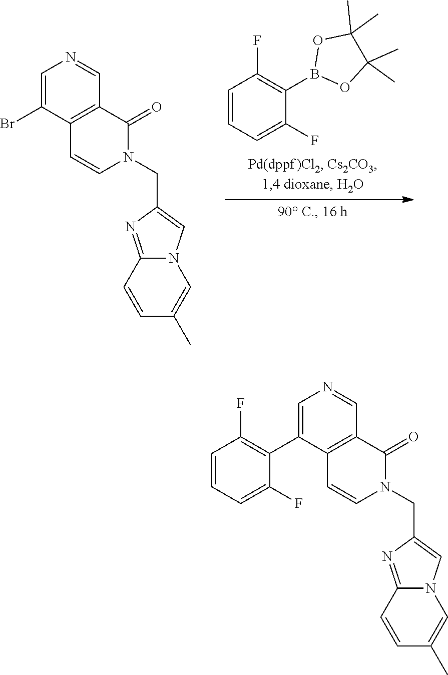 [SMILES: Cc1ccc2nc(Cn3ccc4c(Br)cncc4c3=O)cn2c1 ; CC1(C)OB(c2c(F)cccc2F)OC1(C)C ; Cc1ccc2nc(Cn3ccc4c(-c5c(F)cccc5F)cncc4c3=O)cn2c1]

The title compound was prepared using the procedure described for example 11 with the following modification: the reaction was performed with 2,6-difluorophenyl boronic acid pinacol ester to give 5-(2,6-difluorophenyl)-2-({6-methylimidazo[1,2-a]pyridin-2-yl}methyl)-1,2-dihydro-2,7-naphthyridin-1-one (yield: 5%) as an off-white solid. ¹H NMR (400 MHz, DMSO-d₆): δ 9.45 (s, 1H), 8.74 (s, 1H), 8.29 (s, 1H), 7.87 (d, J=7.6 Hz, 1H), 7.81 (s, 1H), 7.69-7.61 (m, 1H), 7.42-7.30 (m, 3H), 7.08 (dd, J=9.2, 1.7 Hz, 1H), 6.14 (d, J=7.6 Hz, 1H), 5.28 (s, 2H), 2.24 (s, 3H). MS: m/z: 403 [M+H]⁺

### Synthetic Example 30: Synthesis of 5-(2-fluoro-6-methylphenyl)-2-((6-methylimidazo[1,2-a]pyridin-2-yl)methyl)-2,7-naphthyridin-1(2H)-one. (Compound No. 30)

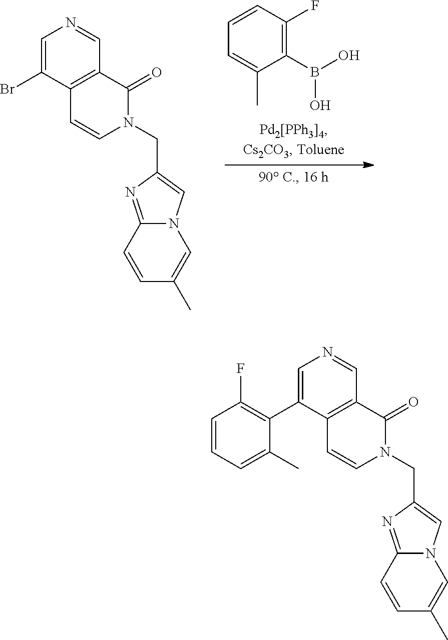 [SMILES: Cc1ccc2nc(Cn3ccc4c(Br)cncc4c3=O)cn2c1 ; Cc1cccc(F)c1B(O)O ; Cc1ccc2nc(Cn3ccc4c(-c5c(C)cccc5F)cncc4c3=O)cn2c1]

The title compound was prepared using the procedure described for example 11 with the following modification: the reaction was performed with 2-fluoro-6-methyl phenyl boronic acid) to give 5-(2-fluoro-6-methylphenyl)-2-({6-methylimidazo[1,2-a]pyridin-2-yl}methyl)-1,2-dihydro-2,7-naphthyridin-1-one (yield: 24%) as an off-white solid. ¹H NMR (400 MHz, DMSO-d₆): δ 9.44 (s, 1H), 8.62 (s, 1H), 8.30 (s, 1H), 7.83 (s, 1H), 7.81 (s, 1H), 7.46 (td, J=7.6, 6.4 Hz, 1H), 7.39 (d, J=9.2 Hz, 1H), 7.27 (d, J=7.6 Hz, 1H), 7.22 (t, J=8.4 Hz, 1H), 7.08 (dd, J=9.2, 1.2 Hz, 1H), 5.97 (d, J=7.2 Hz, 1H), 5.27 (d, J=8.0 Hz, 2H), 2.24 (s, 3H), 2.02 (s, 3H). MS: m/z: 399 [M+H]⁺

### Synthetic Example 31: Synthesis of 5-(2-methoxyphenyl)-2-((6-methylimidazo[1,2-a]pyridin-2-yl)methyl)-2,7-naphthyridin-1(2H)-one. (Compound No. 31)

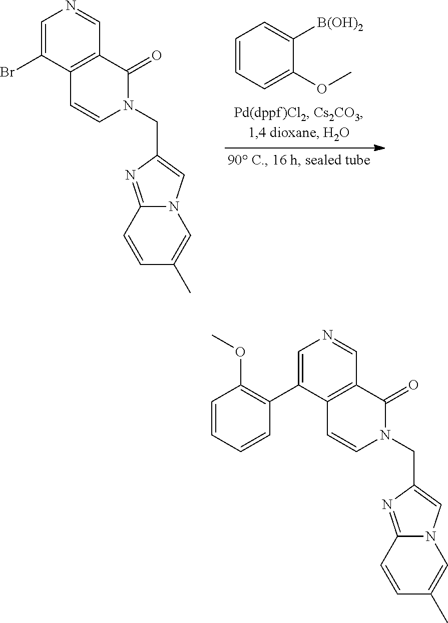 [SMILES: Cc1ccc2nc(Cn3ccc4c(Br)cncc4c3=O)cn2c1 ; COc1ccccc1B(O)O ; COc1ccccc1-c1cncc2c(=O)n(Cc3cn4cc(C)ccc4n3)ccc12]

The title compound was prepared using the procedure described for example 11 with the following modification: the reaction was performed with 2-methoxyphenyl boronic acid to give 5-(2-methoxyphenyl)-2-({6-methylimidazo[1,2-a]pyridin-2-yl}methyl)-1,2-dihydro-2,7-naphthyridin-1-one (yield: 19%) as an off-white solid. ¹H NMR (400 MHz, DMSO-d₆): δ 9.36 (s, 1H), 8.55 (s, 1H), 8.29 (s, 1H), 7.79 (s, 1H), 7.76 (d, J=7.6 Hz, 1H), 7.51-7.47 ((m, 1H), 7.39 (d, J=9.2 Hz, 1H), 7.25 (dd, J=7.6, 1.6 Hz, 1H), 7.20 (d, J=8.4 Hz, 1H), 7.12-7.06 (m, 2H), 6.10 (d, J=7.6 Hz, 1H), 5.26 (d, J=2.0 Hz, 2H), 3.70 (s, 3H), 2.24 (m, 3H). MS: m/z: 397 [M+H]⁺

### Synthetic Example 32: Synthesis of 5-(3,4-dimethoxyphenyl)-2-((6-methylimidazo[1,2-a]pyridin-2-yl)methyl)-2,7-naphthyridin-1(2H)-one. (Compound No. 32)

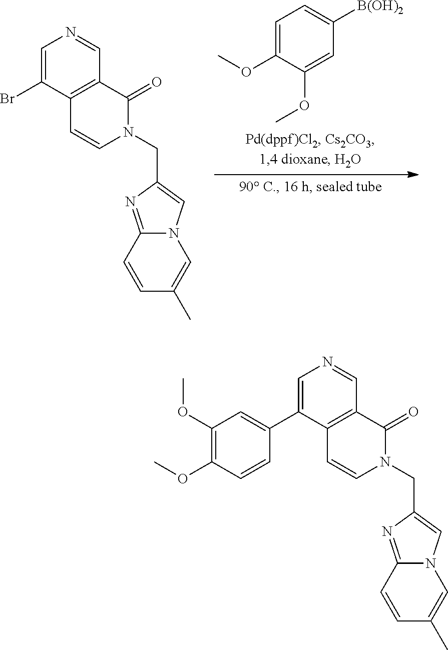

The title compound was prepared using the procedure described for example 11 with the following modification: the reaction was performed with 3,4-dimethoxyphenyl boronic acid to give 5-(4-methoxyphenyl)-2-({6-methylimidazo[1,2-a]pyridin-2-yl}methyl)-1,2-dihydro-2,7-naphthyridin-1-one (yield: 16%) as off white solid. ¹H NMR (400 MHz, DMSO-d₆): δ 9.36 (d, J=0.8 Hz, 1H), 8.66 (s, 1H), 8.29 (t, J=0.8 Hz, 1H), 7.83 (s, 1H), 7.81 (d, J=4.4 Hz, 1H), 7.39 (d, J=9.2 Hz, 1H), 7.13 (s, 1H), 7.10-7.09 (m, 2H), 7.00 (dd, J=8.0, 2.0 Hz, 1H), 6.56 (d, J=8.0 Hz, 1H), 5.28 (s, 2H), 3.83 (s, 3H), 3.80 (s, 3H), 2.24 (s, 3H). MS: m/z: 427 [M+H]⁺

### Synthetic Example 33: Synthesis of 5-benzyl-2-((6-methylimidazo[1,2-a]pyridin-2-yl)methyl)-2,7-naphthyridin-1(2H)-one. (Compound No. 33)

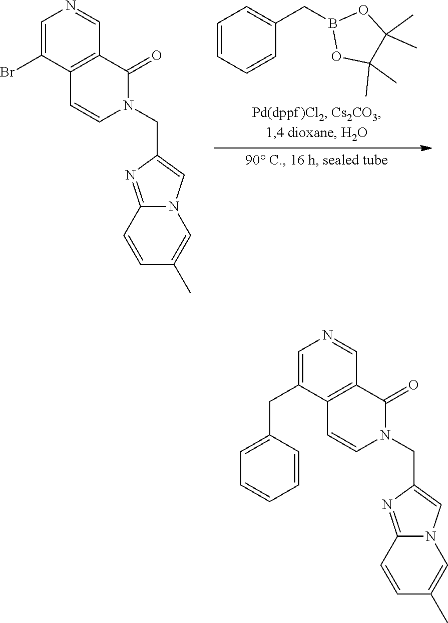 [SMILES: Cc1ccc2nc(Cn3ccc4c(Br)cncc4c3=O)cn2c1 ; CC1(C)OB(Cc2ccccc2)OC1(C)C ; Cc1ccc2nc(Cn3ccc4c(Cc5ccccc5)cncc4c3=O)cn2c1]

The title compound was prepared using the procedure described for example 11 with the following modification: the reaction was performed with benzylboronic acid pinacol ester to give 5-benzyl-2-({6-methylimidazo[1,2-a]pyridin-2-yl}methyl)-1,2-dihydro-2,7-naphthyridin-1-one (yield: 23%) as an off-white solid. ¹H NMR (400 MHz, DMSO-d₆): δ 9.27 (s, 1H), 8.67 (s, 1H), 8.27 (s, 1H), 7.82 (d, J=7.6 Hz, 1H), 7.77 (s, 1H), 7.38 (d, J=9.2 Hz, 1H), 7.29-7.17 (m, 5H), 7.07 (dd, J=9.2, 1.6 Hz, 1H), 6.70 (d, J=7.6 Hz, 1H), 5.24 (s, 2H), 4.23 (s, 2H), 2.23 (s, 3H). MS: m/z: 381 [M+H]⁺

### Synthetic Example 34: Synthesis of 5-(6-aminopyridin-3-yl)-2-((6-methylimidazo[1,2-a]pyridin-2-yl)methyl)-2,7-naphthyridin-1(2H)-one. (Compound No. 34)

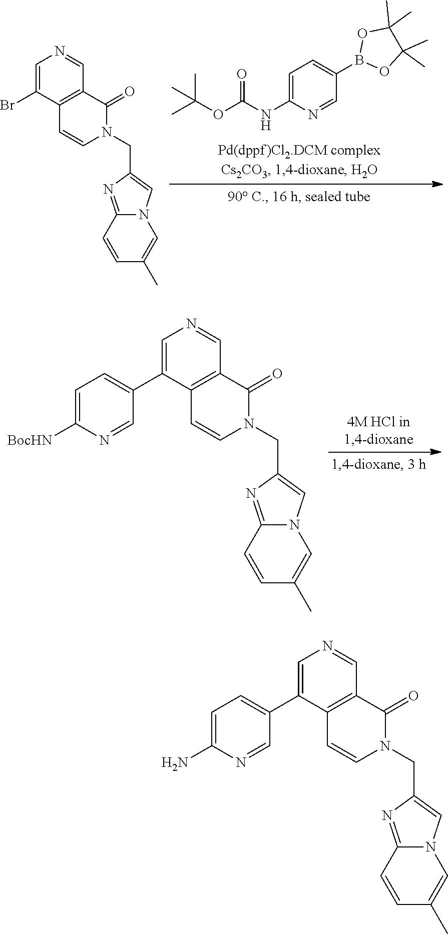

Step 1: The intermediate compound was prepared using the procedure described for example 11 with the following modification: the reaction was performed with tert-butyl (5-(4,4,5,5-tetramethyl-1,3,2-dioxaborolan-2-yl)pyridin-2-yl)carbamate to give tert-butyl N-{5-[7-({6-methylimidazo[1,2-a]pyridin-2-yl}methyl)-8-oxo-7,8-dihydro-2,7-naphthyridin-4-yl]pyridin-2-yl}carbamate (yield: 63%) as a gummy white solid. MS: m/z: 483 [M+H]⁺

Step 2: The title compound was prepared using the procedure described for example 4, step 2 with the following modification: the reaction was performed with tert-butyl N-{5-[7-({6-methylimidazo[1,2-a]pyridin-2-yl}methyl)-8-oxo-7,8-dihydro-2,7-naphthyridin-4-yl]pyridin-2-yl}carbamate (80 mg, 166 μmol) and purified by reverse phase prep HPLC (Column: X-Bridge C18 (4.6*150).5 um, 0-95% CH₃CN/H2O) to give 5-(6-aminopyridin-3-yl)-2-({6-methylimidazo[1,2-a]pyridin-2-yl}methyl)-1,2-dihydro-2,7-naphthyridin-1-one (yield:19%) as an off white solid. ¹H NMR (400 MHz, DMSO-d₆): δ 9.32 (s, 1H), 8.61 (s, 1H), 8.29 (s, 1H), 8.01 (d, J=2.0 Hz, 1H), 7.83 (d, J=7.6 Hz, 1H), 779 (s, 1H), 7.53 (dd, J=8.8, 2.4 Hz, 1H), 7.40 (d, J=9.2 Hz, 1H), 7.09 (dd, J=9.2, 1.6 Hz, 1H), 6.59 (d, J=8.4 Hz, 1H), 6.51 (d, J=4.0 Hz, 1H), 6.25 (s, 2H), 5.28 (s, 2H), 2.24 (s, 3H). MS: m/z: 383 [M+H]⁺

### Synthetic Example 35: Synthesis of 5-(4-(aminomethyl)phenyl)-2-((6-methylimidazo[1,2-a]pyridin-2-yl)methyl)-2,7-naphthyridin-1(2H)-one. (Compound No. 35)

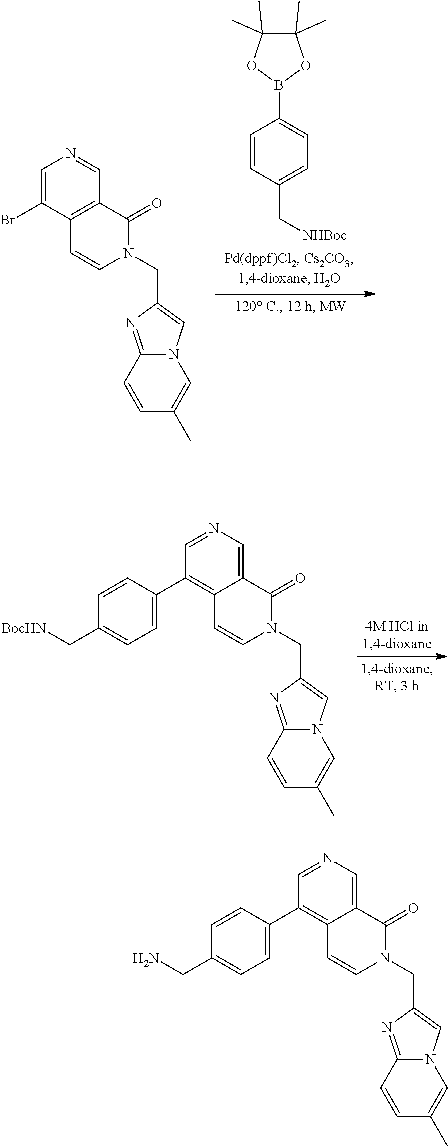

Step 1: The intermediate compound was prepared using the procedure described for example 11 with the following modification: the reaction was performed with tert-butyl (4-(4,4,5,5-tetramethyl-1,3,2-dioxaborolan-2-yl)benzyl)carbamate to give tert-butyl N-({4-[7-({6-methylimidazo[1,2-a]pyridin-2-yl}methyl)-8-oxo-7,8-dihydro-2,7-naphthyridin-4-yl]phenyl}methyl)carbamate (yield: 81%) as a pale brown solid. MS: m/z: 496 [M+H]⁺

Step 2: The title compound was prepared using the procedure described for example 34, step 2 with the following modification: the reaction was performed with tert-butyl N-({4-[7-({6-methylimidazo[1,2-a]pyridin-2-yl}methyl)-8-oxo-7,8-dihydro-2,7-naphthyridin-4-yl]phenyl}methyl)carbamate (0.1 g, 202 μmol) and purified by prep-HPLC (COLUMN: ACQUITY BEH C18 (2.1*50*1.7 u), 0-95% CH₃CN/H2O) to give 5-[4-(aminomethyl)phenyl]-2-({6-methylimidazo[1,2-a]pyridin-2-yl}methyl)-1,2-dihydro-2,7-naphthyridin-1-one (35 mg, yield: 44%) as an off-white solid. ¹H NMR (400 MHz, DMSO-d₆): δ 9.44 (s, 1H), 8.71 (s, 1H), 8.64 (s, 1H), 8.36 (s, 2H), 8.22 (s, 1H), 7.93 (d, J=7.6 Hz, 1H), 7.81 (d, J=9.2 Hz, 1H), 7.72 (dd, J=9.2, 1.6 Hz, 1H), 7.66 (d, J=8.0 Hz, 2H), 7.56 (d, J=8.0 Hz, 2H), 6.48 (d, J=7.6 Hz, 1H), 5.47 (s, 2H), 4.15 (t, J=5.6 Hz, 2H), 2.38 (s, 3H). MS: m/z: 396.4 [M+H]⁺

### Synthetic Example 36: Synthesis of N-(2-(7-((6-methylimidazo[1,2-a]pyridin-2-yl)methyl)-8-oxo-7,8-dihydro-2,7-naphthyridin-4-yl)phenyl)acetamide. (Compound No. 36)

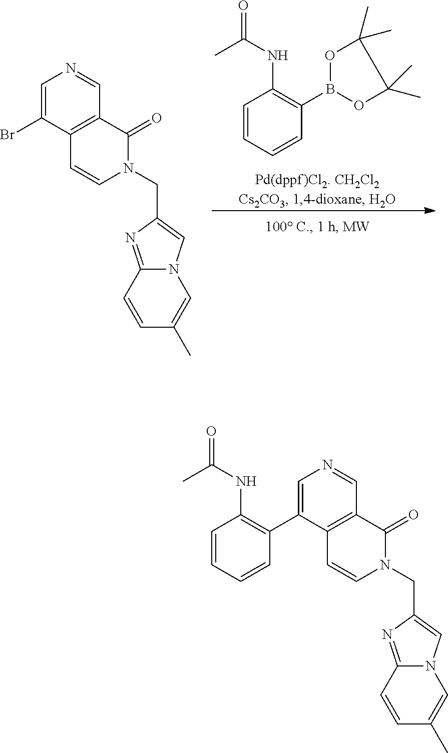 [SMILES: Cc1ccc2nc(Cn3ccc4c(Br)cncc4c3=O)cn2c1 ; CC(=O)Nc1ccccc1B1OC(C)(C)C(C)(C)O1 ; CC(=O)Nc1ccccc1-c1cncc2c(=O)n(Cc3cn4cc(C)ccc4n3)ccc12]

The title compound was prepared using the procedure described for example 11 with the following modification: the reaction was performed with N-(2-(4,4,5,5-tetramethyl-1,3,2-dioxaborolan-2-yl)phenyl)acetamide to give N-{2-[7-({6-methylimidazo[1,2-a]pyridin-2-yl}methyl)-8-oxo-7,8-dihydro-2,7-naphthyridin-4-yl]phenyl}acetamide(yield: 7%) as an off-white solid. ¹H NMR (400 MHz, DMSO-d₆): δ 9.38 (s, 1H), 9.14 (s, 1H), 8.54 (s, 1H), 8.40 (s, 1H), 7.91 (s, 1H), 7.79 (d, J=7.6 Hz, 1H), 7.65 (d, J=8.0 Hz, 1H), 7.48-7.45 (m, 2H), 7.34-7.25 (m, 3H), 6.05 (d, J=7.6 Hz, 1H), 5.31 (s, 2H), 2.28 (s, 3H), 1.72 (s, 3H). MS: m/z: 424 [M+H]⁺

### Synthetic Example 37: Synthesis of 5-(4-(hydroxymethyl)phenyl)-2-((6-methylimidazo[1,2-a]pyridin-2-yl)methyl)-2,7-naphthyridin-1(2H)-one. (Compound No. 37)

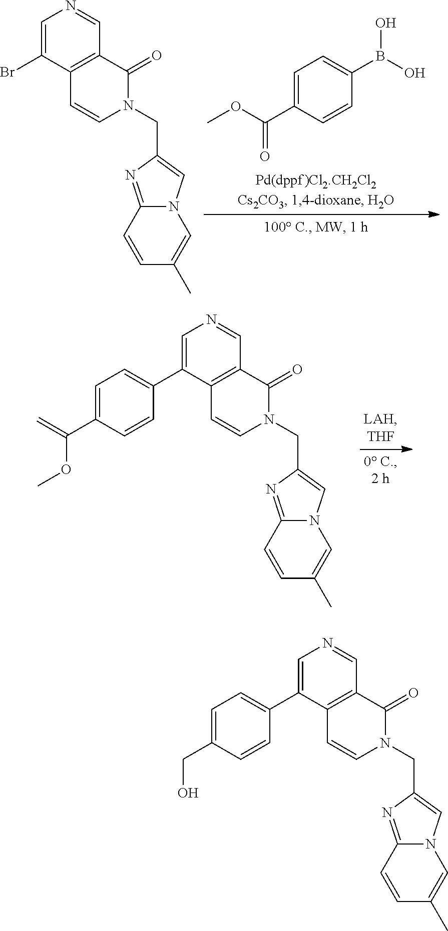

Step 1: The intermediate compound was prepared using the procedure described for example 11 with the following modification: the reaction was performed with (4-(methoxycarbonyl)phenyl)boronic acid to give methyl 4-[7-({6-methylimidazo[1,2-a]pyridin-2-yl}methyl)-8-oxo-7,8-dihydro-2,7-naphthyridin-4-yl]benzoate (yield: 50%) as a gummy white solid. MS: m/z: 425 [M+H]⁺

Step 2: 1M Lithium aluminium hydride in THF (0.09 mL, 113.04 μmol) was added dropwise to a stirred solution of methyl 4-[7-({6-methylimidazo[1,2-a]pyridin-2-yl}methyl)-8-oxo-7,8-dihydro-2,7-naphthyridin-4-yl]benzoate (40 mg, 94.2 μmol) in THF (10 mL) at 0° C. The reaction mixture was allowed to warm to room temperature and stirred for an additional 2 h. The reaction mixture was quenched with saturated Na₂SO₄ solution (10 mL), filtered through a celite pad, and the filtrate was extracted with DCM (30 mL×2). The organic layers were combined, dried over anhydrous sodium sulphate, concentrated in vacuo and purified by mPLC (SiO₂, 60% EtOAc/Hexane) to get 5-[4-(hydroxymethyl)phenyl]-2-({6-methylimidazo[1,2-a]pyridin-2-yl}methyl)-1,2-dihydro-2,7-naphthyridin-1-one (8.2 mg, yield: 22%) as a white solid. ¹H NMR (400 MHz, DMSO-d₆): δ 9.38 (s, 1H), 8.65 (s, 1H), 8.29 (s, 1H), 7.84 (d, J=7.6 Hz, 1H), 7.80 (s, 1H), 7.49 (d, J=8.4 Hz, 2H), 7.45 (d, J=8.4 Hz, 2H), 7.39 (d, J=9.2 Hz, 1H), 7.08 (dd, J=9.2, 1.6 Hz, 1H), 6.48 (d, J=7.6 Hz, 1H), 5.28 (s, 2H), 4.59 (d, J=5.2 Hz, 2H), 2.24 (s, 3H).

MS: m/z: 397 [M+H]⁺

### Synthetic Example 38: Synthesis of N-(5-(7-((6-methylimidazo[1,2-a]pyridin-2-yl)methyl)-8-oxo-7,8-dihydro-2,7-naphthyridin-4-yl)pyridin-2-yl)acetamide. (Compound No. 38)

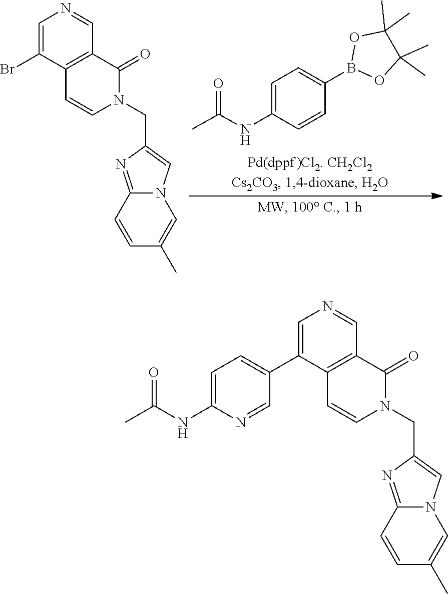

The title compound was prepared using the procedure described for example 11 with the following modification: the reaction was performed with N-(5-(4,4,5,5-tetramethyl-1,3,2-dioxaborolan-2-yl)pyridin-2-yl)acetamide to give 5-[2-(hydroxymethyl)phenyl]-2-({6-methylimidazo[1,2-a]pyridin-2-yl}methyl)-1,2-dihydro-2,7-naphthyridin-1-one (yield: 8%) as an off-white solid. ¹H NMR (400 MHz, DMSO-d₆): δ 10.70 (s, 1H), 9.40 (s, 1H), 8.70 (s, 1H), 8.43 (d, J=2.4 Hz, 1H), 8.24 (d, J=8.4 Hz, 1H), 7.94 (dd, J=8.4, 2.4 Hz, 1H), 7.87 (d, J=7.6 Hz, 1H), 7.81 (s, 1H), 7.39 (d, J=9.2 Hz, 1H), 7.08 (dd, J=9.2, 1.6 Hz, 1H), 6.49 (d, J=7.6 Hz, 1H), 6.29 (s, 1H), 5.29 (s, 2H), 2.24 (s, 3H), 2.14 (s, 3H). MS: m/z: 397 [M+H]⁺

### Synthetic Example 39: Synthesis of 5-(1H-indazol-7-yl)-2-((6-methylimidazo[1,2-a]pyridin-2-yl)methyl)-2,7-naphthyridin-1(2H)-one. (Compound No. 39)

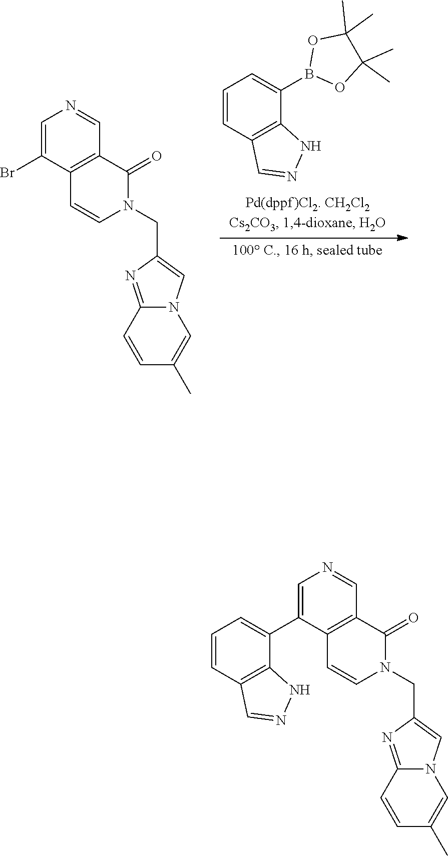 [SMILES: Cc1ccc2nc(Cn3ccc4c(Br)cncc4c3=O)cn2c1 ; CC1(C)OB(c2cccc3cn[nH]c23)OC1(C)C ; Cc1ccc2nc(Cn3ccc4c(-c5cccc6cn[nH]c56)cncc4c3=O)cn2c1]

The title compound was prepared using the procedure described for example 11 with the following modification: the reaction was performed with 1H-indazole-7 boronic acid pinacol ester to give 5-(1H-indazol-7-yl)-2-({6-methylimidazo[1,2-a]pyridin-2-yl}methyl)-1,2-dihydro-2,7-naphthyridin-1-one (yield: 13%) as an off-white solid. ¹H NMR (400 MHz, DMSO-d₆): δ 12.92 (s, 1H), 9.47 (d, J=0.4 Hz, 1H), 8.75 (s, 1H), 8.30 (t, J=1.2 Hz, 1H), 8.20 (s, 1H), 7.90 (dd, J=8.0, 0.8 Hz, 1H), 7.80 (d, J=4.0 Hz, 1H), 7.78 (s, 1H), 7.40-7.34 (m, 2H), 7.29-7.26 (m, 1H), 7.08 (dd, J=9.2, 1.6 Hz, 1H), 6.04 (d, J=7.6 Hz, 1H), 5.28 (s, 2H), 2.24 (d, J=0.8 Hz, 3H). MS: m/z: 407 [M+H]⁺

### Synthetic Example 40: Synthesis of 5-(6-methoxypyridin-3-yl)-2-((6-methylimidazo[1,2-a]pyridin-2-yl)methyl)-2,7-naphthyridin-1(2H)-one. (Compound No. 40)

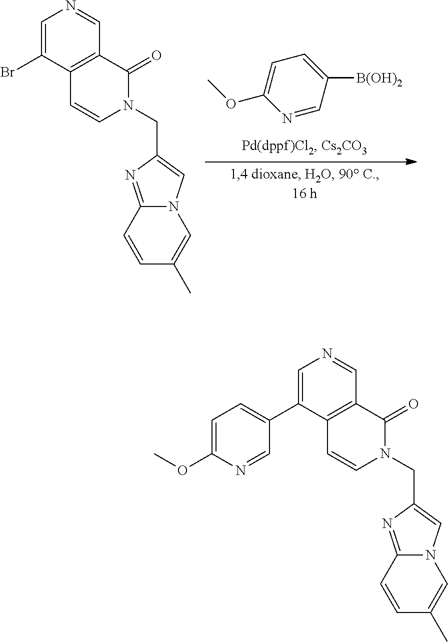

The title compound was prepared using the procedure described for example 11 with the following modification: the reaction was performed with 2-methoxypyridine-5-boronic acid to give 5-(6-methoxypyridin-3-yl)-2-({6-methylimidazo[1,2-a]pyridin-2-yl}methyl)-1,2-dihydro-2,7-naphthyridin-1-one (yield: 11%) as an off-white solid. ¹H NMR (400 MHz, DMSO-d₆): δ 9.39 (s, 1H), 8.67 (s, 1H), 8.30 (s, 1H), 7.89-7.85 (m, 2H), 7.80 (s, 1H), 7.59-7.46 (m, 1H), 7.39 (d, J=9.2 Hz, 1H), 7.08 (dd, J=9.2, 1.7 Hz, 1H), 7.00 (d, J=8.4 Hz, 1H), 6.45 (d, J=7.6 Hz, 1H), 5.29 (s, 2H), 3.93 (s, 3H), 2.24 (s, 3H); MS: m/z: 398 [M+H]⁺

### Synthetic Example 41: Synthesis of 2-((6-methylimidazo[1,2-a]pyridin-2-yl)methyl)-5-(1-(methylsulfonyl)-1H-pyrazol-4-yl)-2,7-naphthyridin-1(2H)-one.(Compound No. 41)

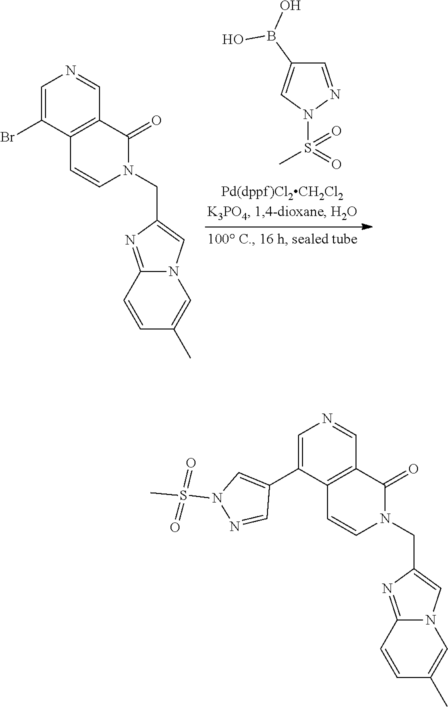

The title compound was prepared using the procedure described for example 11 with the following modification: the reaction was performed with 1-(methylsulfonyl)4-pyrazolyl)boronic acid to give 5-(1-methanesulfonyl-1H-pyrazol-4-yl)-2-({6-methylimidazo[1,2-a]pyridin-2-yl}methyl)-1,2-dihydro-2,7-naphthyridin-1-one (yield: 22%) as an off-white solid. ¹H NMR (400 MHz, DMSO-d₆): δ 9.36 (s, 1H), 8.82 (s, 1H), 8.69 (s, 1H), 8.35 (s, 1H), 8.29 (s, 1H), 7.91 (d, J=7.6 Hz, 1H), 7.81 (s, 1H), 7.39 (d, J=9.2 Hz, 1H), 7.09 (dd, J=9.2, 1.6 Hz, 1H), 6.75 (d, J=7.6 Hz, 1H), 5.30 (s, 2H), 3.66 (s, 3H), 2.24 (s, 3H). MS: m/z: 435 [M+H]⁺

### Synthetic Example 42: Synthesis of 5-(1-cyclopropyl-1H-pyrazol-4-yl)-2-((6-methylimidazo[1,2-a]pyridin-2-yl)methyl)-2,7-naphthyridin-1(2H)-one. (Compound No. 42)

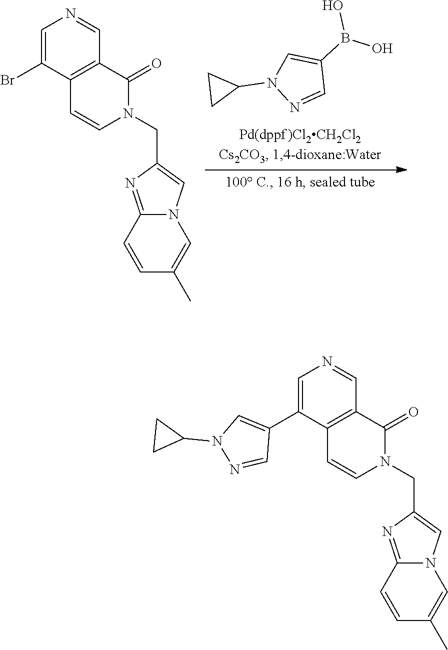 [SMILES: Cc1ccc2nc(Cn3ccc4c(Br)cncc4c3=O)cn2c1 ; OB(O)c1cnn(C2CC2)c1 ; Cc1ccc2nc(Cn3ccc4c(-c5cnn(C6CC6)c5)cncc4c3=O)cn2c1]

The title compound was prepared using the procedure described for example 11 with the following modification: the reaction was performed with 2-cyclopropylpyrazole-4-boronic acid to give 5-(1-cyclopropyl-1H-pyrazol-4-yl)-2-({6-methylimidazo[1,2-a]pyridin-2-yl}methyl)-1,2-dihydro-2,7-naphthyridin-1-one (yield: 34%) as an off-white solid. ¹H NMR (400 MHz, DMSO-d₆): δ 9.27 (s, 1H), 8.73 (s, 1H), 8.29 (s, 1H), 8.23 (s, 1H), 7.85 (d, J=7.6 Hz, 1H), 7.80 (s, 2H), 7.40 (d, J=9.2 Hz, 1H), 7.08 (dd, J=9.2, 1.6 Hz, 1H), 6.80 (d, J=7.6 Hz, 1H), 5.29 (s, 2H), 3.82 (tt, J=7.2, 3.6 Hz, 1H), 2.24 (s, 3H), 1.14-1.11 (m, 2H), 1.03-1.00 (m, 2H). MS: m/z: 397 [M+H]⁺

### Synthetic Example 43: Synthesis of 5-(1-benzyl-1H-pyrazol-4-yl)-2-((6-methylimidazo[1,2-a]pyridin-2-yl)methyl)-2,7-naphthyridin-1(2H)-one. (Compound No. 43)

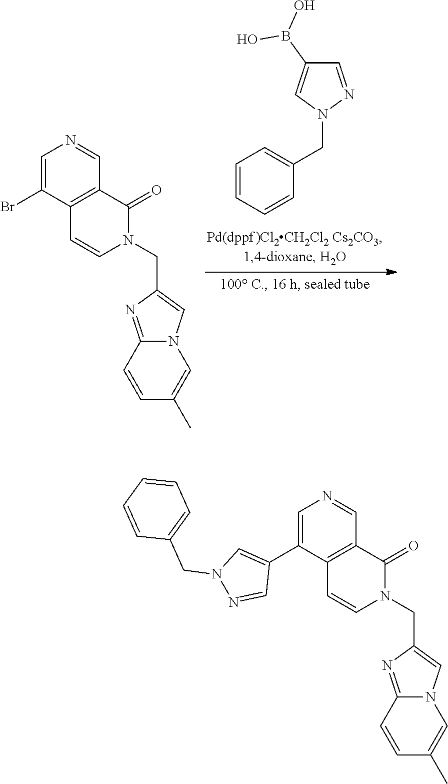 [SMILES: Cc1ccc2nc(Cn3ccc4c(Br)cncc4c3=O)cn2c1 ; OB(O)c1cnn(Cc2ccccc2)c1 ; Cc1ccc2nc(Cn3ccc4c(-c5cnn(Cc6ccccc6)c5)cncc4c3=O)cn2c1]

The title compound was prepared using the procedure described for example 11 with the following modification: the reaction was performed with 1H-pyrazole-1-benzyl-4-boronic acid to give 5-(1-benzyl-1H-pyrazol-4-yl)-2-({6-methylimidazo[1,2-a]pyridin-2-yl}methyl)-1,2-dihydro-2,7-naphthyridin-1-one (yield: 26%) as an off-white solid. ¹H NMR (400 MHz, DMSO-d₆): δ 9.28 (d, J=0.4 Hz, 1H), 8.75 (s, 1H), 8.35 (d, J=0.4 Hz, 1H), 8.29 (s, 1H), 7.88-7.86 (m, 2H), 7.79 (s, 1H), 7.40-7.35 (m, 3H), 7.32-7.30 (m, 3H), 7.08 (dd, J=9.2, 1.6 Hz, 1H), 6.79 (d, J=7.6 Hz, 1H), 5.42 (s, 2H), 5.29 (s, 2H), 2.24 (s, 3H). MS: m/z: 447 [M+H]⁺

### Synthetic Example 44: Synthesis of 5-(2-(hydroxymethyl) phenyl)-2-((6-methylimidazo[1,2-a]pyridin-2-yl)methyl)-2,7-naphthyridin-1(2H)-one. (Compound No. 44)

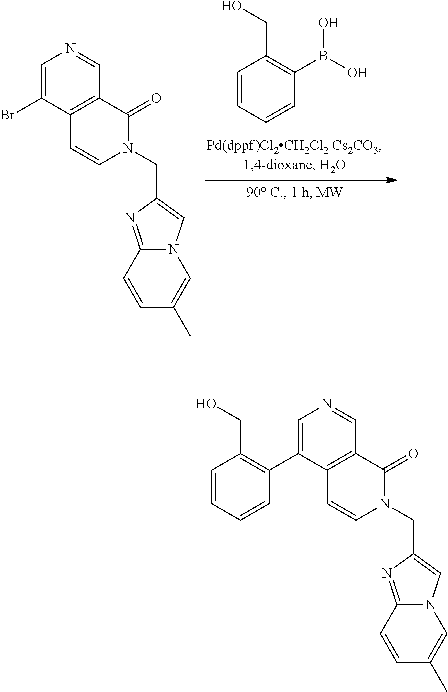 [SMILES: Cc1ccc2nc(Cn3ccc4c(Br)cncc4c3=O)cn2c1 ; OCc1ccccc1B(O)O ; Cc1ccc2nc(Cn3ccc4c(-c5ccccc5CO)cncc4c3=O)cn2c1]

The title compound was prepared using the procedure described for example 11 with the following modification: the reaction was performed with 2-(hydroxymethyl) phenyl boronic acid to give 5-[2-(hydroxymethyl) phenyl]-2-({6-methylimidazo[1,2-a]pyridin-2-yl}methyl)-1,2-dihydro-2,7-naphthyridin-1-one (49 mg, yield: 91%) as an off white solid. ¹H NMR (400 MHz, DMSO-d₆): δ 9.39 (d, J=0.8 Hz, 1H), 8.57 (s, 1H), 8.29 (d, J=0.4 Hz, 1H), 7.80 (s, 1H), 7.78 (s, 1H), 7.65 (dd, J=7.6, 1.2 Hz, 1H), 7.51 (td, J=7.6, 1.2 Hz, 1H), 7.41-7.39 (m, 2H), 7.20 (dd, J=7.6, 1.4 Hz, 1H), 7.08 (dd, J=9.2, 1.6 Hz, 1H), 5.99 (dd, J=7.6, 0.8 Hz, 1H), 5.26 (d, J=10.0 Hz, 2H), 5.09 (s, 1H), 4.18 (s, 2H), 2.24 (s, 3H). MS: m/z: 397 [M+H]⁺

### Synthetic Example 45: 5-ethynyl-2-((6-methylimidazo[1,2-a]pyridin-2-yl)methyl)-2,7-naphthyridin-1(2H)-one. (Compound No. 45)

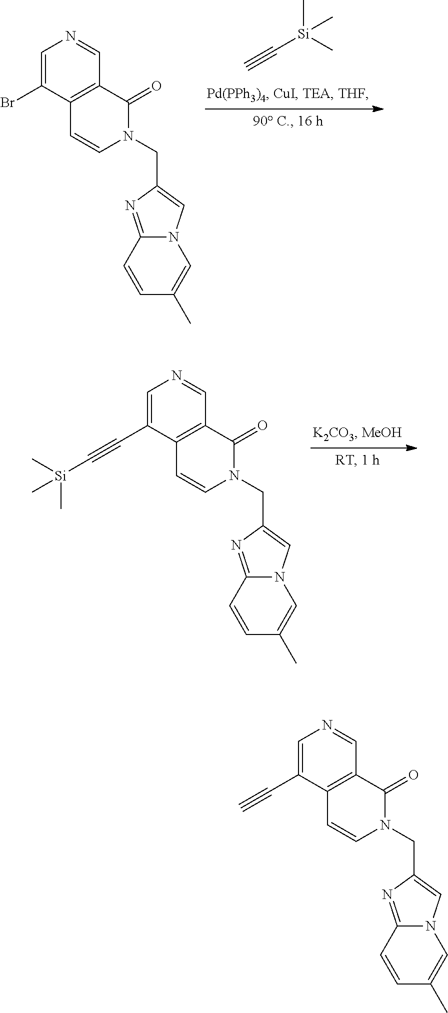

Step 1: A solution of 5-bromo-2-({6-methylimidazo[1,2-a]pyridin-2-yl}methyl)-1,2-dihydro-2,7-naphthyridin-1-one(100 mg 27.1 mol) in THF(10 mL) at room temperature was purged with nitrogen for 10 min, followed by the addition of triethylamine (82.2 mg, 81.3 μmol), ethynyl trimethylsilane (26.6 mg, 27.1 μmol) and Pd(PPh₃)₄ (19.9 mg, 2.71 μmol). The reaction mixture was heated at 90° C. for 16 h, cooled to room temperature, filtered through a celite pad, and the filtrate was concentrated in vacuo to give 2-((6-methylimidazo[1,2-a]pyridin-2-yl)methyl)-5-((trimethylsilyl)ethynyl)-2,7-naphthyridin-1(2H)-one (60 mg, yield: 58%) as a pale brown MS: m/z: 371[M+H]⁺

Step 2: Potassium carbonate (46.6 mg, 466 μmol) was added to a stirred solution of 2-({6-methylimidazo[1,2-a]pyridin-2-yl}methyl)-5-[trimethylsily)ethynyl]-1,2-dihydro-2,7-naphthyridin-1-one (90.0 mg, 233 μmol) in methanol (5 mL) at room temperature. After 3 h, the reaction mixture was diluted with water (5 mL) and extracted with DCM (30 mL×2). The organic layers were dried over anhydrous sodium sulphate, filtered, concentrated in vacuo and the residue was purified by reverse phase prep HPLC (COLUMN: Y MC TRIATC18(250*20 mm)5 um, 0-955 CH₃CN/H₂O) to afford 5-ethynyl-2-({6-mehylimidazo[1,2-a]pyridin-2-yl}methyl)-1,2-dihydro-2,7-naphthyridin-1-one (10.4 mg, yield: 26%) as a pale brown solid. ¹H NMR (400 MHz, DMSO-d₆): δ69.34 (s, 1H), 8.91 (s, 1H), 8.66 (s, 1H), 8.25 (s, 1H), 8.02 (d, J=7.2 Hz, 1H), 7.82-7.79 (m, 2H), 6.85 (d, J=7.2 Hz, 1H), 5.47 (s, 2H), 4.88 (s, 1H), 2.38 (s, 3H). MS: m/z: 315 [M+H]⁺

### Synthetic Example 46: 5-(cyclopropyl ethynyl)-2-((6-methylimidazo[1,2-a]pyridin-2-yl)methyl)-2,7-naphthyridin-1(2H)-one. (Compound No. 46)

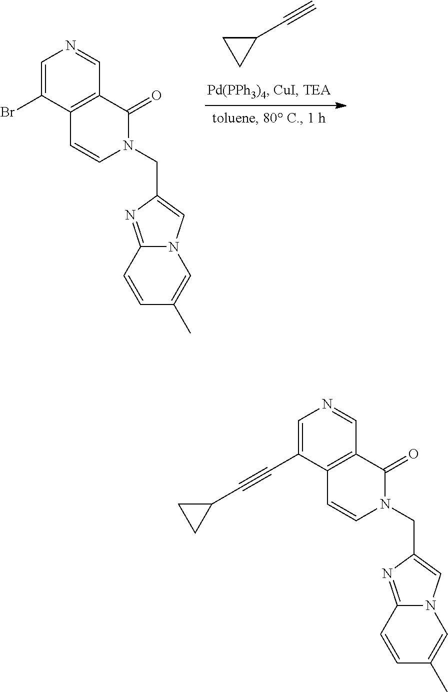

The title compound was prepared using the procedure described for example 45, step 1 with the following modification: the reaction was performed with 2-cyclopropylacetylene to give 5-(2-cyclopropylethynyl)-2-({6-methylimidazo[1,2-a]pyridin-2-yl}methyl)-1,2-dihydro-2,7-naphthyridin-1-one (yield: 19%) as an off-white solid. ¹H NMR (400 MHz, DMSO-d₆) δ 9.22 (s, 1H), 8.73 (s, 1H), 8.28 (s, 1H), 7.94 (d, J=7.6 Hz, 1H), 7.78 (s, 1H), 7.39 (d, J=9.2 Hz, 1H), 7.08 (dd, J=9.2, 1.6 Hz, 1H), 6.74 (d, J=7.6 Hz, 1H), 5.28 (s, 2H), 2.24 (s, 3H), 1.72-1.65 (m, 1H), 1.00-0.96 (m, 2H), 0.88-0.87 (m, 2H). MS: m/z: 354 [M+H]⁺

### Synthetic Example 47: 2-((6-methylimidazo[1,2-a]pyridin-2-yl)methyl)-4-(1H-pyrazol-4-yl)-2,7-naphthyridin-1(2H)-one. (Compound No. 47)

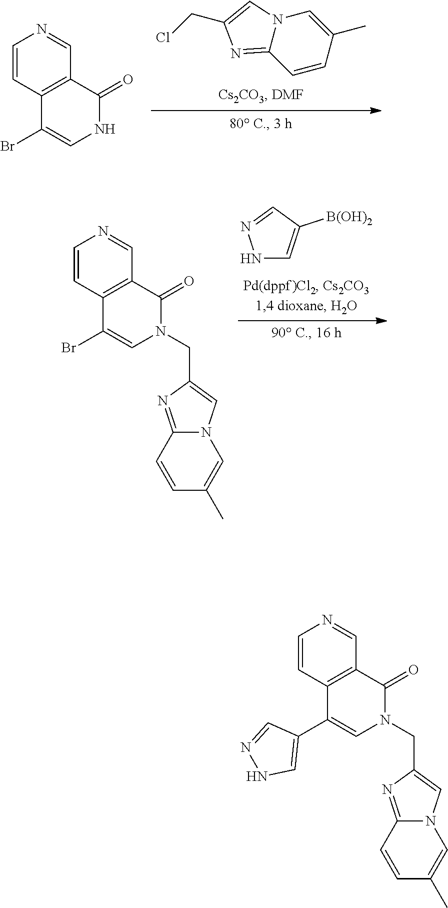 [SMILES: O=c1[nH]cc(Br)c2ccncc12 ; Cc1ccc2nc(CCl)cn2c1 ; Cc1ccc2nc(Cn3cc(Br)c4ccncc4c3=O)cn2c1 ; OB(O)c1cn[nH]c1 ; Cc1ccc2nc(Cn3cc(-c4cn[nH]c4)c4ccncc4c3=O)cn2c1]

Step 1: The intermediate compound was prepared using the procedure described for example 1, step 2 with the following modification: the reaction was performed with 4-bromo-1,2-dihydro-2,7-naphthyridin-1-one to give 4-bromo-2-({6-methylimidazo[1,2-a]pyridin-2-yl}methyl)-1,2-dihydro-2,7-naphthyridin-1-one (yield: 43%) as a white-solid. ¹H NMR (400 MHz, DMSO-d₆): δ 9.36 (d, J=0.8 Hz, 1H), 8.88 (d, J=5.6 Hz, 1H), 8.33 (s, 1H), 8.29 (s, 1H), 7.82 (s, 1H), 7.63 (dd, J=5.6, 0.8 Hz, 1H), 7.41 (d, J=9.2 Hz, 1H), 7.09 (dd, J=9.3, 1.2 Hz, 1H), 5.29 (s, 2H), 2.24 (s, 3H). MS: m/z: 369 [M+H]⁺

Step 2: The title compound was prepared using the procedure described for example 11 with the following modification: the reaction was performed with 1H-pyrazole-4-boronic acid to give 2-({6-methylimidazo[1,2-a]pyridin-2-yl}methyl)-4-(1H-pyrazol-4-yl)-1,2-dihydro-2,7-naphthyridin-1-one (yield: 30%) as an off-white solid. ¹H NMR (400 MHz, DMSO-d₆) δ 13.12 (bs, 1H), 9.42 (s, 1H), 8.76 (d, J=5.6 Hz, 1H), 8.28 (s, 1H), 8.02 (s, 1H), 7.91 (s, 1H), 7.80 (s, 1H), 7.70 (s, 1H), 7.64 (d, J=5.6 Hz, 1H), 7.40 (d, J=8.8 Hz, 1H), 7.07 (d, J=9.3 Hz, 1H), 5.32 (s, 2H), 2.23 (s, 3H). MS: m/z:356.8 [M+H]⁺

### Synthetic Example 48: 4-cyclopropyl-2-((6-methylimidazo[1,2-a]pyridin-2-yl)methyl)-2,7-naphthyridin-1(2H)-one. (Compound No. 48)

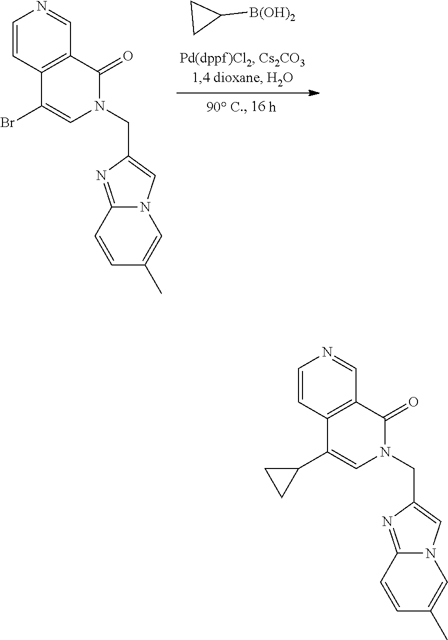 [SMILES: Cc1ccc2nc(Cn3cc(Br)c4ccncc4c3=O)cn2c1 ; OB(O)C1CC1 ; Cc1ccc2nc(Cn3cc(C4CC4)c4ccncc4c3=O)cn2c1]

The title compound was prepared using the procedure described for example 47, step 2 with the following modification: the reaction was performed with cyclopropyl boronic acid to give 4-cyclopropyl-2-({6-methylimidazo[1,2-a]pyridin-2-yl}methyl)-1,2-dihydro-2,7-naphthyridin-1-one (21.7 mg, yield: 35%) as an off-white solid. ¹H NMR (400 MHz, DMSO-d₆): δ 9.36 (d, J=0.8 Hz, 1H), 8.81 (d, J=5.6 Hz, 1H), 8.27 (d, J=0.4 Hz, 1H), 7.88 (dd, J=5.6, 0.8 Hz, 1H), 7.73 (s, 1H), 7.65 (d, J=1.2 Hz, 1H), 7.40 (d, J=9.2 Hz, 1H), 7.07 (dd, J=9.2, 1.6 Hz, 1H), 5.24 (s, 2H), 2.23 (d, J=0.4 Hz, 3H), 1.94-1.86((m, 1H), 0.94-0.84 (m, 2H), 0.60-0.55 (m, 2H). MS: m/z:332 [M+H]⁺

### Synthetic Example 49: 4-methyl-2-((6-methylimidazo[1,2-a]pyridin-2-yl)methyl)-2,7-naphthyridin-1(2H)-one. (Compound No. 49)

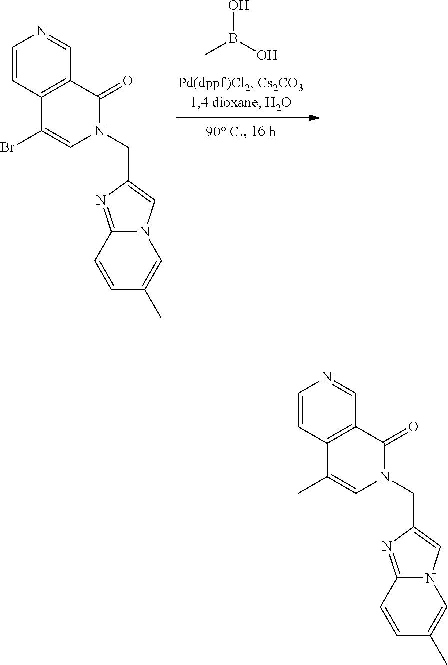

The title compound was prepared using the procedure described for example 47, step 2 with the following modification: the reaction was performed with methyl boronic acid to give 4-methyl-2-({6-methylimidazo[1,2-a]pyridin-2-yl}methyl)-1,2-dihydro-2,7-naphthyridin-1-one (yield: 14%) as a white solid. ¹H NMR (400 MHz, DMSO-d₆): δ 9.37 (d, J=0.8 Hz, 1H), 8.78 (d, J=5.6 Hz, 1H), 8.28 (t, J=0.8 Hz, 1H), 7.76 (s, 1H), 7.70 (d, J=1.2 Hz, 1H), 7.58 (dd, J=5.6, 0.8 Hz, 1H), 7.40 (d, J=9.2 Hz, 1H), 7.08 (dd, J=9.2, 1.6 Hz, 1H), 5.23 (s, 2H), 2.24 (s, 3H), 2.23 (s, 3H). MS: m/z: 304.8 [M+H]⁺

### Synthetic Example 50: 4-benzyl-2-((6-methylimidazo[1,2-a]pyridin-2-yl)methyl)-2,7-naphthyridin-1(2H)-one. (Compound No. 50)

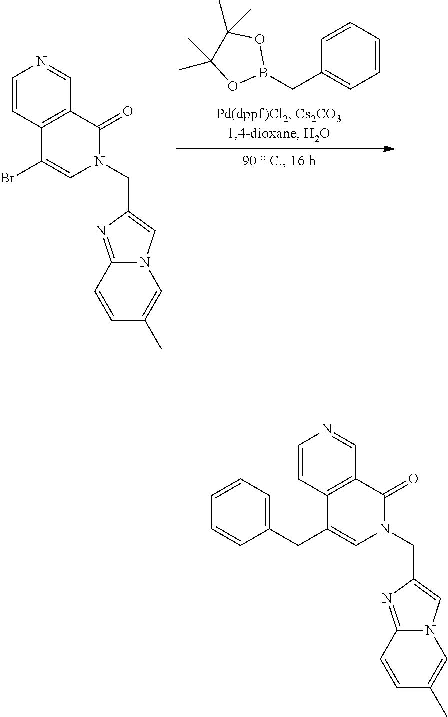 [SMILES: Cc1ccc2nc(Cn3cc(Br)c4ccncc4c3=O)cn2c1 ; CC1(C)OB(Cc2ccccc2)OC1(C)C ; Cc1ccc2nc(Cn3cc(Cc4ccccc4)c4ccncc4c3=O)cn2c1]

The title compound was prepared using the procedure described for example 47, step 2 with the following modification: the reaction was performed with benzyl boronic acid pinacol ester to give 4-benzyl-2-({6-methylimidazo[1,2-a]pyridin-2-yl}methyl)-1,2-dihydro-2,7-naphthyridin-1-one (yield: 20%) as an off-white solid. ¹H NMR (400 MHz, DMSO-d₆): δ 9.38 (s, 1H), 8.68 (d, J=5.6 Hz, 1H), 8.43 (s, 1H), 7.95 (s, 1H), 7.86 (s, 1H), 7.56 (d, J=9.2 Hz, 1H), 7.50 (dd, J=5.6, 0.8 Hz, 1H), 7.35-7.24 (m, 5H), 7.21-7.16 (m, 1H), 5.35 (s, 2H), 4.04 (s, 2H), 2.30 (s, 3H). MS: m/z: 381 [M+H]⁺

### Synthetic Example 51: Synthesis of 4-ethyl-2-((6-methylimidazo[1,2-a]pyridin-2-yl)methyl)-2,7-naphthyridin-1(2H)-one. (Compound No. 51)

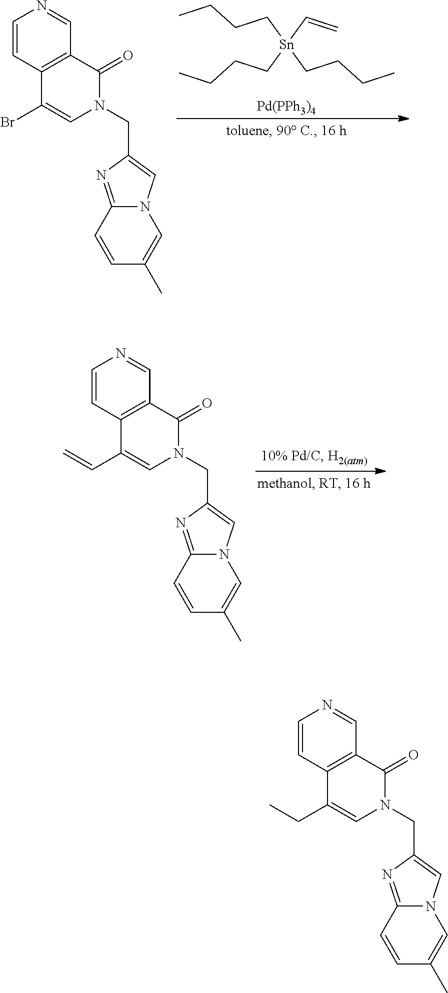 [SMILES: Cc1ccc2nc(Cn3cc(Br)c4ccncc4c3=O)cn2c1 ; C=[CH][Sn]([CH2]CCC)([CH2]CCC)[CH2]CCC ; C=Cc1cn(Cc2cn3cc(C)ccc3n2)c(=O)c2cnccc12 ; CCc1cn(Cc2cn3cc(C)ccc3n2)c(=O)c2cnccc12 ; "Pd(PPh3)4"]

Step 1: A solution of 4-bromo-2-({6-methylimidazo[1,2-a]pyridin-2-yl}methyl)-1,2-dihydro-2,7-naphthyridin-1-one(10 mg, 27.1 μmol) in toluene (2 mL) was purged with argon for 20 min, followed by the addition of tributyl(ethenyl)stannane (12.9 mg, 40.6 μmol), and Pd (PPh₃)₄(3.13 mg, 2.71 μmol) and heated at 90° C. for 16 h. The reaction mixture was cooled to room temperature and filtered through a celite pad. The filtrate was concentrated in vacuo and triturated with diethyl ether (20 mL×2) to give 4-ethenyl-2-({6-methylimidazo[1,2-a]pyridin-2-yl}methyl)-1,2-dihydro-2,7-naphthyridin-1-one (60 mg, 190 μmol, yield: 70%) as a pale-yellow solid. MS: m/z: 317 [M+H]⁺

Step 2: To a stirred solution of 4-ethenyl-2-({6-methylimidazo[1,2-a]pyridin-2-yl}methyl)-1,2-dihydro-2,7-naphthyridin-1-one(40 mg, 126 μmol) in methanol (10 mL) was added 10% palladium (40 mg, 376 μmol) at room temperature under an inert atmosphere. The resultant reaction mixture was hydrogenated at 25 psi for 16 h. The reaction mixture was filtered through a celite pad, and the filtrate was concentrated in vacuo and purified by Prep-HPLC (Column: YMC TRIART C18(250*20 MM*5 u), 0-95% ACN/H₂O) to afford 4-ethyl-2-({6-methylimidazo[1,2-a]pyridin-2-yl}methyl)-1,2-dihydro-2,7-naphthyridin-1-one (8.5 mg, yield: 21%) as a white solid. ¹H NMR (400 MHz, DMSO-d₆): δ 9.42 (d, J=0.4 Hz, 1H), 8.81 (d, J=5.6 Hz, 1H), 8.58 (s, 1H), 8.14 (s, 1H), 7.76-7.68 (m, 3H), 7.64 (d, J=9.6 Hz, 1H), 5.40 (s, 2H), 2.70-2.67 (m, 2H), 2.35 (s, 3H), 1.22 (t, J=7.2 Hz, 3H). MS: m/z: 319.2 [M+H]⁺

### Synthetic Example 52: Synthesis of 2-((6-methylimidazo[1,2-b]pyridazin-2-yl)methyl)-5-phenyl-2,7-naphthyridin-1(2H)-one. (Compound No. 52)

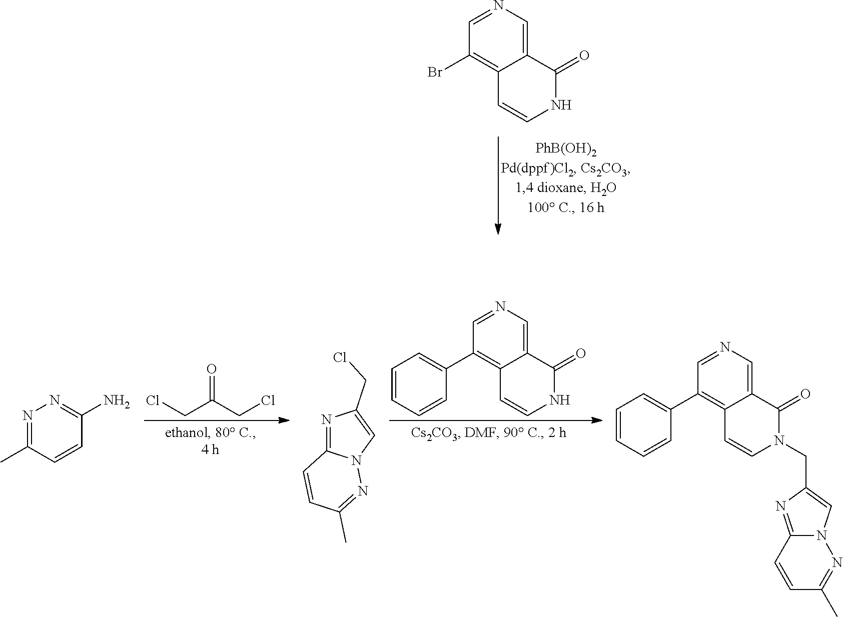

Step 1: The intermediate compound was prepared using the procedure described for example 1, step 1 with the following modification: the reaction was performed with 6-methylpyridazin-3-amine (250 mg, 2.29 mmol) to give 2-(chloromethyl)-6-methylimidazo[1,2-b]pyridazine (yield: 24%) as brown solid. MS: m/z: 182 [M+H]⁺

Step 2: A solution of 5-bromo-1,2-dihydro-2,7-naphthyridin-1-one (0.1 g, 444 μmol) in a mixture of dioxane: H₂O (4:1)(2.5 mL) was purged with nitrogen gas for 15 min, followed by the addition of phenylboronic acid (65 m, 533 μmol), Cs₂CO₃ (290 mg, 889 μmol) and Pd(dppf)Cl₂.DCM complex (72.5 mg, 88.9 μmol) and heated at 100° C. for 16 h. The reaction mixture was cooled to room temperature, concentrated in vacuo and purified by column chromatography (SiO₂, 50% EtOAc/Hexane) to give 5-phenyl-1,2-dihydro-2,7-naphthyridin-1-one (80 mg, yield: 81%) as a pale-brown solid. MS: m/z: 225 [M+H]⁺

Step 3: The title compound was prepared using the procedure described for example 1, step 2 with the following modification: the reaction was performed with 5-phenyl-1,2-dihydro-2,7-naphthyridin-1-one (60.0 mg, 270 μmol) to give 2-({6-methylimidazo[1,2-b]pyridazin-2-yl}methyl)-5-phenyl-1,2-dihydro-2,7-naphthyridin-1-one (10 mg, yield: 16%) as off-white solid. ¹H NMR (400 MHz, DMSO-d₆): δ 9.38 (d, J=0.8 Hz, 1H), 8.66 (s, 1H), 8.15 (s, 1H), 7.93 (d, J=9.2 Hz, 1H), 7.85 (d, J=7.6 Hz, 1H), 7.57-7.48 (m, 5H), 7.12 (d, J=9.2 Hz, 1H), 6.46 (dd, J=7.6, 0.8 Hz, 1H), 5.33 (s, 2H), 2.50 (s, 3H). MS: m/z: 368 [M+H]⁺

### Synthetic Example 53: Synthesis of 2-(benzo[d]oxazol-2-ylmethyl)-5-phenyl-2,7-naphthyridin-1(2H)-one. (Compound No. 53)

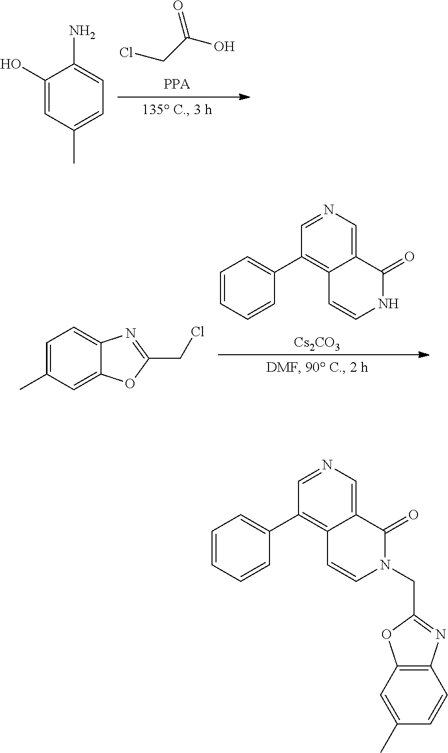

Step 1: A solution of 2-amino-5-methylphenol (500 mg, 4.06 mmol) and 2-chloroacetic acid (959 mg, 10.1 mmol) in poly phosphoric acid (5 mL) was heated at 135° C. for 3 h. The reaction mixture was cooled to room temperature, quenched with saturated NaHCO₃ (20 mL), and extracted with ethyl acetate (60 mL×2). The combined organic layers were dried over anhydrous sodium sulphate, filtered, concentrated in vacuo and purified by column chromatography (SiO₂, 10% EtOAc/Hexane) to afford 2-(chloromethyl)-6-methyl-1,3-benzoxazole (110 mg, yield: 15%) as a pale-yellow liquid.

Step 2: The title compound was prepared using the procedure described for example 1, step 2 with the following modification: the reaction was performed with 1,2-dihydro-2,7-naphthyridin-1-one and 2-(chloromethyl)-6-methyl-1,3-benzoxazole to give 2-[(1-oxo-1,2-dihydro-2,7-naphthyridin-2-yl)methyl]imidazo[1,2-a]pyridine-6-carbaldehyde (yield: 34%) as a pale brown solid. ¹H NMR (400 MHz, DMSO-d₆): δ 9.37 (s, 1H), 8.72 (s, 1H), 7.90 (d, J=7.6 Hz, 1H), 7.60-7.50 (m, 7H), 7.18 (dd, J=9.2, 1.2 Hz, 1H), 6.56 (d, J=7.6 Hz, 1H), 5.55 (s, 2H), 2.43 (s, 3H). MS: m/z: 368 [M+H]⁺

### Synthetic Example 54: Synthesis of 2-((5-methylbenzo[d]oxazol-2-yl)methyl)-5-phenyl-2,7-naphthyridin-1(2H)-one. (Compound No. 54)

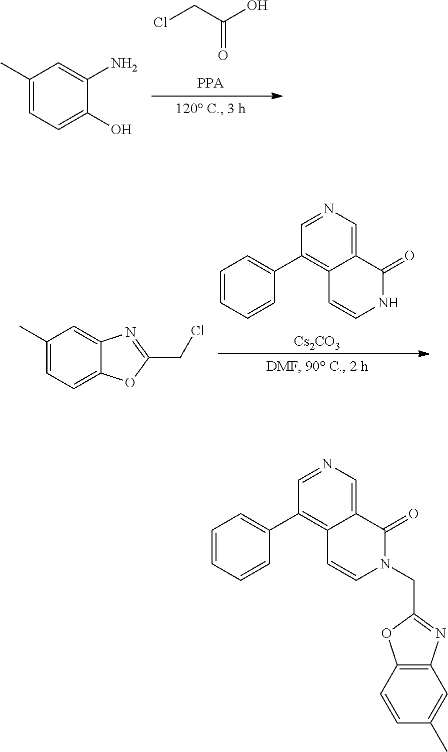

Step 1: The intermediate compound was prepared using the procedure described for example 53, step 1 with the following modification: the reaction was performed with 2-amino-4-methylphenol (0.5 g, 4.06 mmol) to give 2-(chloromethyl)-6-methyl-1,3-benzoxazole (yield: 16%) as a yellow-liquid.

Step 2: The title compound was prepared using the procedure described for example 1, step 2 with the following modification: the reaction was performed with 5-phenyl-1,2-dihydro-2,7-naphthyridin-1-one and 2-(chloromethyl)-6-methyl-1,3-benzoxazole to give 2-[(6-methyl-1,3-benzoxazol-2-yl)methyl]-5-phenyl-1,2-dihydro-2,7-naphthyridin-1-one (yield: 33%) as an off-white solid. ¹H NMR (400 MHz, DMSO-d₆) δ 9.37 (s, 1H), 8.72 (s, 1H), 7.91 (d, J=7.6 Hz, 1H), 7.63-7.49 (m, 6H), 7.48 (s, 1H), 7.20 (d, J=8.0 Hz, 1H), 6.57 (d, J=7.6 Hz, 1H), 5.55 (s, 2H), 2.40 (s, 3H). MS: m/z: 368 [M+H]⁺

### Synthetic Example 55: Synthesis of 2-((6-methyl-1H-benzo[d]imidazol-2-yl)methyl)-5-phenyl-2,7-naphthyridin-1(2H)-one. (Compound No. 55)

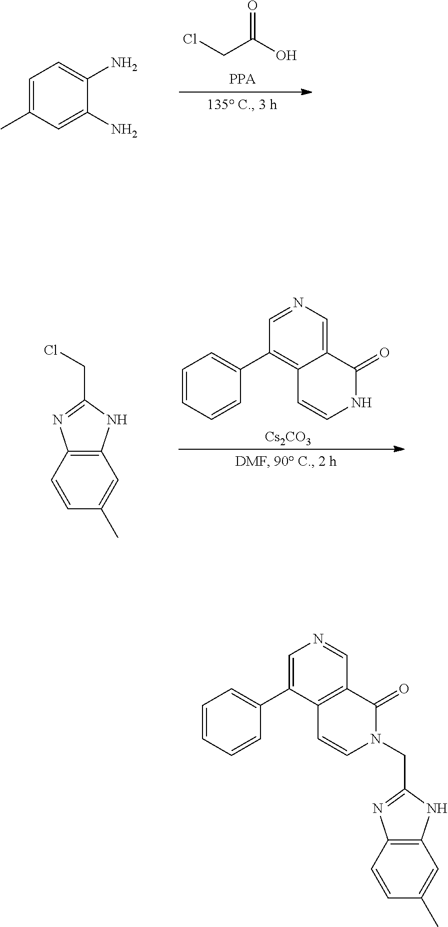

Step 1: The intermediate compound was prepared using the procedure described for example 53, step 1 with the following modification: the reaction was performed with 4-methylbenzene-1,2-diamine to give 2-(chloromethyl)-6-methyl-1H-1,3-benzodiazole (yield: 30%) as a pale brown solid. MS: m/z: 181 [M+H]⁺

Step 2: The title compound was prepared using the procedure described for example 1, step 2 with the following modification: the reaction was performed with 5-phenyl-1,2-dihydro-2,7-naphthyridin-1-one and 2-(chloromethyl)-6-methyl-1H-1,3-benzodiazole to give 2-[(6-methyl-1H-1,3-benzodiazol-2-yl)methyl]-5-phenyl-1,2-dihydro-2,7-naphthyridin-1-one (yield: 12%) as a pale brown solid. ¹H NMR (400 MHz, DMSO-d₆): δ 12.36 (s, 1H), 9.39 (s, 1H), 8.69 (s, 1H), 7.86 (d, J=7.6 Hz, 1H), 7.60-7.56 (m, 2H), 7.53-7.50 (m, 3H), 7.36 (s, 1H), 7.27 (s, 1H), 6.96 (d, J=6.8 HZ, 1H), 6.52 (d, J=7.6 Hz, 1H), 5.42 (s, 2H), 2.38 (s, 3H). MS: m/z: 367 [M+H]⁺

### Synthetic Example 56: Synthesis of 2-((6-methylimidazo[1,2-a]pyrimidin-2-yl)methyl)-5-phenyl-2,7-naphthyridin-1(2H)-one. (Compound No. 56)

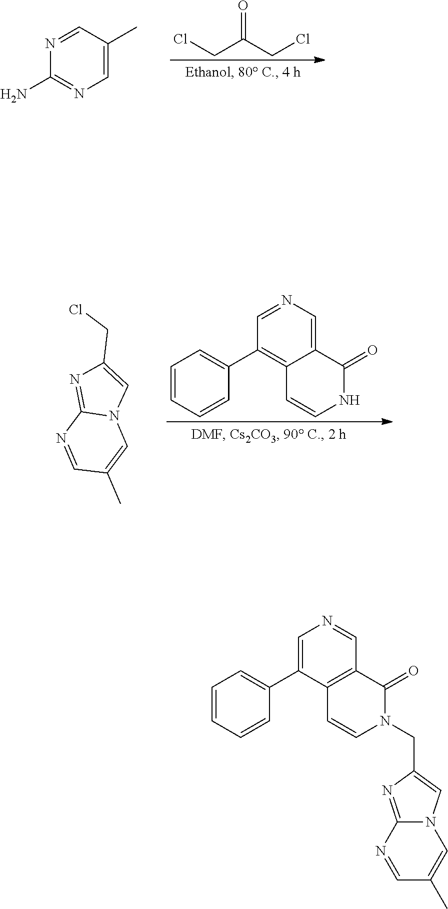

Step 1: The intermediate compound was prepared using the procedure described for example 1, step 1 with the following modification: the reaction was performed with 5-methylpyrimidin-2-amine (250 mg, 2.29 mmol) to give 2-(chloromethyl)-6-methylimidazo[1,2-a]pyrimidine (100 mg, yield: 24%) as a pale-yellow solid. MS: m/z: 182 [M+H]⁺

Step 2: The title compound was prepared using the procedure described for example 1, step 2 with the following modification: the reaction was performed with 5-phenyl-1,2-dihydro-2,7-naphthyridin-1-one and 2-(chloromethyl)-6-methylimidazo[1,2-a]pyrimidine to give 2-({6-methylimidazo[1,2-a]pyrimidin-2-yl}methyl)-5-phenyl-1,2-dihydro-2,7-naphthyridin-1-one (yield: 13%) as a brown solid. ¹H NMR (400 MHz, DMSO-d₆): δ 9.39 (s, 1H), 8.71 (s, 1H), 8.67 (s, 1H), 8.40 (d, J=2.0 Hz, 1H), 7.87 (d, J=7.6 Hz, 1H), 7.77 (s, 1H), 7.61-7.46 (m, 5H), 6.48 (dd, J=7.6, 0.8 Hz, 1H), 5.32 (s, 2H), 2.27 (d, J=0.8 Hz, 3H). MS: m/z: 368 [M+H]⁺

### Synthetic Example 57: Synthesis of 2-((7-methylimidazo[1,2-a]pyrimidin-2-yl)methyl)-5-phenyl-2,7-naphthyridin-1(2H)-one. (Compound No. 57)

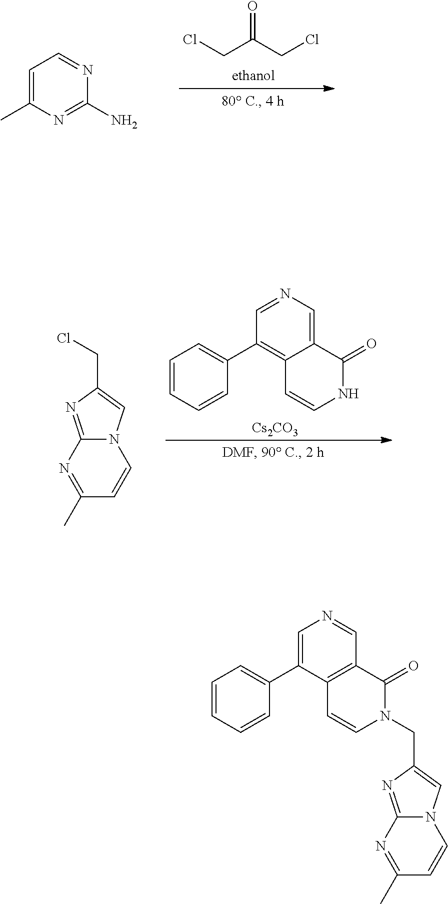

Step 1: The intermediate compound was prepared using the procedure described for example 1, step 1 with the following modification: the reaction was performed with 4-methylpyrimidin-2-amine (100 mg, 916 μmol) to give 2-(chloromethyl)-7-methylimidazo[1,2-a]pyrimidine (25.0 mg, yield: 15%) as a pale-brown solid. MS: m/z: 182 [M+H]⁺

Step 2: The title compound was prepared using the procedure described for example 1, step 2 with the following modification: the reaction was performed with 5-phenyl-1,2-dihydro-2,7-naphthyridin-1-one and 2-(chloromethyl)-7-methylimidazo[1,2-a]pyrimidine to give 2-({7-methylimidazo[1,2-a]pyrimidin-2-yl}methyl)-5-phenyl-1,2-dihydro-2,7-naphthyridin-1-one (yield: 9%) as an off-white solid. ¹H NMR (400 MHz, DMSO-d₆): δ 9.39 (s, 1H), 8.75 (d, J=6.8 Hz, 1H), 8.67 (s, 1H), 7.86 (d, J=7.6 Hz, 1H), 7.72 (s, 1H), 7.58-7.54((m, 5H), 6.93 (d, J=7.2 Hz, 1H), 6.48 (d, J=7.2 Hz, 1H), 5.31 (s, 2H), 2.51 (s, 3H). MS: m/z: 368 [M+H]⁺

### Synthetic Example 58: Synthesis of 2-((2-methylimidazo[2,1-b]thiazol-6-yl)methyl)-5-phenyl-2,7-naphthyridin-1(2H)-one. (Compound No. 58)

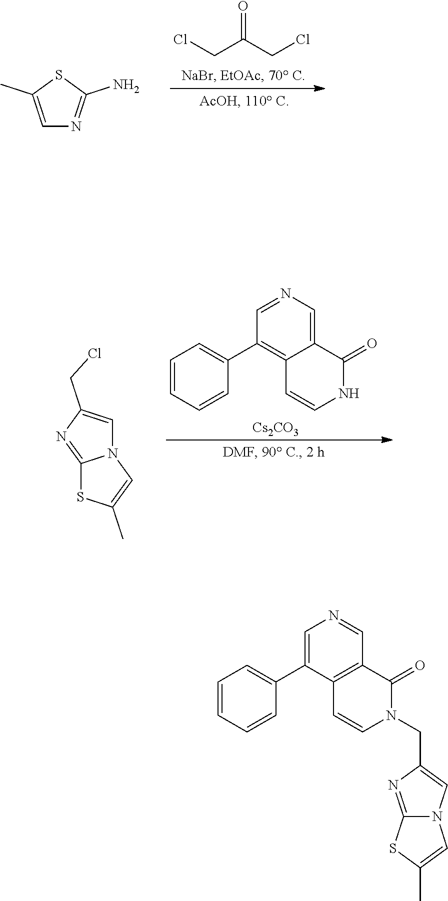

Step 1: 1,3-dichloropropan-2-one (400 mg, 1.45 mmol) was added to a stirred solution of 5-methyl-1,3-thiazol-2-amine (300 mg, 2.63 mmol) in ethyl acetate (8 mL) followed by the addition of sodium bromide (135 mg, 1.32 mmol). The reaction mixture was heated at 70° C. for 16 h, cooled to room temperature, concentrated in vacuo and the resultant residue was dissolved in acetic acid (5 mL) and heated at 110° C. for 2 h. The reaction mixture was cooled to room temperature, concentrated in vacuo and triturated with diethylether (15 mL) to obtain 6-(chloromethyl)-2-methylimidazo[2,1-b][1,3]thiazole (200 mg, yield: 41%) as a pale brown solid. MS: m/z: 187 [M+H]⁺

Step 2: The title compound was prepared using the procedure described for example 1, step 2 with the following modification: the reaction was performed with 5-phenyl-1,2-dihydro-2,7-naphthyridin-1-one and 6-(chloromethyl)-2-methylimidazo[2,1-b][1,3]thiazole to give 2-({2-methylimidazo[2,1-b][1,3]thiazol-6-yl}methyl)-5-phenyl-1,2-dihydro-2,7-naphthyridin-1-one (yield: 9%) as a pale brown solid. ¹H NMR (400 MHz, DMSO-d₆): δ 10.64 (s, 1H), 8.66 (d, J=2.4 Hz, 1H), 8.29 (s, 1H), 7.95 (dd, J=7.6, 2.4 Hz, 1H), 7.69 (s, 1H), 7.43-7.34 (m, 6H), 7.07 (dd, J=9.2, 1.6 Hz, 1H), 6.46 (d, J=7.6 Hz, 1H), 5.31 (s, 2H), 4.56 (d, J=5.2 Hz, 2H), 2.25 (d, J=0.4 Hz, 3H). MS: m/z: 373 [M+H]⁺

### Synthetic Example 59: Synthesis of 2-((2-methyl-1H-benzo[d]imidazol-6-yl)methyl)-5-phenyl-2,7-naphthyridin-1(2H)-one. (Compound No. 59)

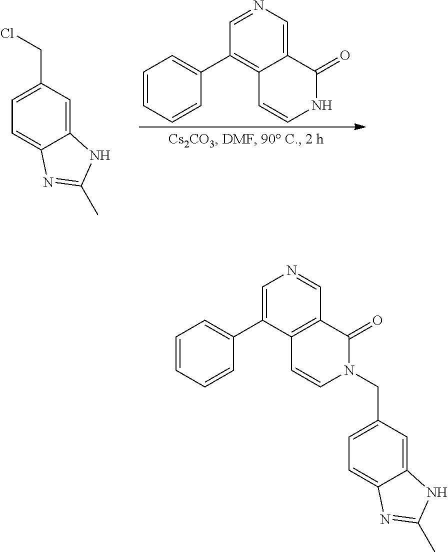 [SMILES: Cc1nc2ccc(CCl)cc2[nH]1 ; O=c1[nH]ccc2c(-c3ccccc3)cncc12 ; Cc1nc2ccc(Cn3ccc4c(-c5ccccc5)cncc4c3=O)cc2[nH]1]

The title compound was prepared using the procedure described for example 1, step 2 with the following modification: the reaction was performed with 5-phenyl-1,2-dihydro-2,7-naphthyridin-1-one to give 2-[(2-methyl-1H-1,3-benzodiazol-6-yl)methyl]-5-phenyl-1,2-dihydro-2,7-naphthyridin-1-one (yield: 19%) as an off-white solid. ¹H NMR (400 MHz, DMSO-d₆): δ 12.17 (s, 1H), 9.42 (s, 1H), 8.65 (s, 1H), 7.86 (d, J=7.6 Hz, 1H), 7.56-7.47 (m, 6H), 7.40 (d, J=8.0 Hz, 1H), 7.17 (dd, J=8.4, 1.6 Hz, 1H), 6.45 (d, J=7.6 Hz, 1H), 5.28 (s, 2H), 2.45 (s, 3H). MS: m/z: 367 [M+H]⁺

### Synthetic Example 60: Synthesis of 2-((1,5-dimethyl-1H-indol-2-yl)methyl)-5-phenyl-2,7-naphthyridin-1(2H)-one. (Compound No. 60)

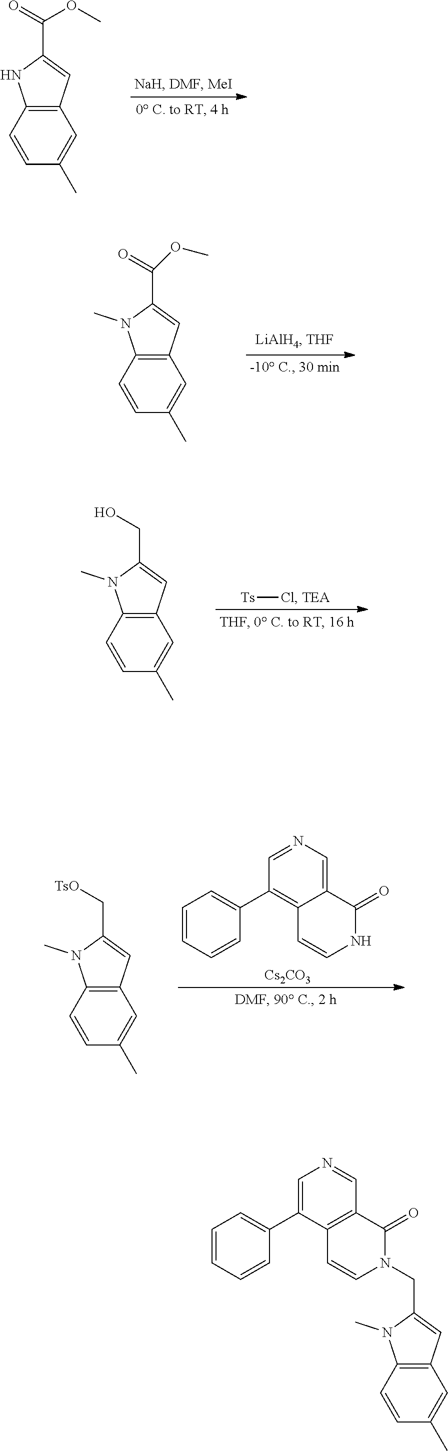

Step 1: Methyl 5-methyl-1H-indole-2-carboxylate (0.5 g, 2.64 mmd) was added to a suspension of 60% sodium hydride (127 mg, 5.29 mmd) in N, N-dimethylformamide (8 mL) at 0° C. After 20 min, iodomethane (563 mg, 3.96 mmol) was added to the reaction mixture and it was allowed to warm to room temperature and stirred for 4 h. The reaction mixture was quenched with ice-cold water (5 mL) and the resultant solid was filtered, dried to give methyl 1,5-dimethyl-1H-indole-2-carboxylate(300 mg, yield: 57%) as a white solid. MS: m/z: 204 [M+H]⁺

Step 2: LiAlH₄ (213 mg, 2.95 mmol) was added to a stirred solution of methyl 1,5-dimethyl-1H-indole-2-carboxylate (30 mg, 1.48 mmol) in THF (2 mL) at −10° C. After 30 min, the reaction mixture was allowed to warm to stir at room temperature and stirred for 4 h. The reaction mixture was cooled to 0° C., quenched with saturated disodium sulphate (10 mL), passed through a celite pad, and the filtrate was extracted with ethyl acetate (40 mL×2). The combined organic layers were dried over anhydrous sodium sulphate, filtered and concentrated to give (1,5-dimethyl-1H-indol-2-yl)methanol (40 mg, yield: 70%) as a white solid. MS: m/z: 176 [M+H]⁺

Step 3: Triethylamine (312 mg, 3.08 mmol) was added to a stirred solution of (1,5-dimethyl-1H-indol-2-yl)methanol (180 mg, 1.03 mmol) in DCM (2 mL) at 0° C. followed by the addition of 4-methylbenzene-1-sulfonyl chloride (392 mg, 2.05 mmol) after 10 min. The reaction mixture was allowed to warm to room temperature, stirred for an additional 16 h, concentrated in vacuo, and the residue was diluted with ice-water (10 mL), and extracted with DCM (50 mL×2). The combined organic layers were dried over sodium sulphate, filtered, concentrated in vacuo and purified by column chromatography (SiO₂, 15% EtOAc/hexane) to give (1,5-dimethyl-1H-indol-2-yl)methyl 4-methylbenzene-1-sulfonate(150 mg yield: 44%).

MS: m/z: 330 [M+H]⁺

Step 4: The title compound was prepared using the procedure described for example 1, step 2 with the following modification: the reaction was performed with 5-phenyl-1,2-dihydro-2,7-naphthyridin-1-one and (1,5-dimethyl-1H-indol-2-yl)methyl 4-methylbenzene-1-sulfonate and purified by reverse phase prep-HPLC (COLUMN: YMCTRIART; 0-95% CH₃CN/H₂O) to afford 2-[(1,5-dimethyl-1H-indol-2-yl)methyl]-5-phenyl-1,2-dihydro-2,7-naphthyridin-1-one (yield: 9%). ¹H NMR (400 MHz, DMSO-d₆): δ 9.44 (s, 1H), 8.69 (s, 1H), 7.72 (d, J=7.7 Hz, 1H), 7.56-7.47 (m, 5H), 7.31 (d, J=8.4 Hz, 1H), 7.26 (s, 1H), 6.97 (dd, J=8.4, 1.7 Hz, 1H), 6.50 (d, J=7.6 Hz, 1H), 6.29 (d, J=2.4 Hz, 1H), 5.43 (s, 2H), 3.73 (s, 3H), 2.34 (s, 3H). MS: (m/z): 380 [M+H]⁺

### Synthetic Example 61: Synthesis of 2-((1,6-dimethyl-1H-indol-2-yl)methyl)-5-phenyl-2,7-naphthyridin-1(2H)-one. (Compound No. 61)

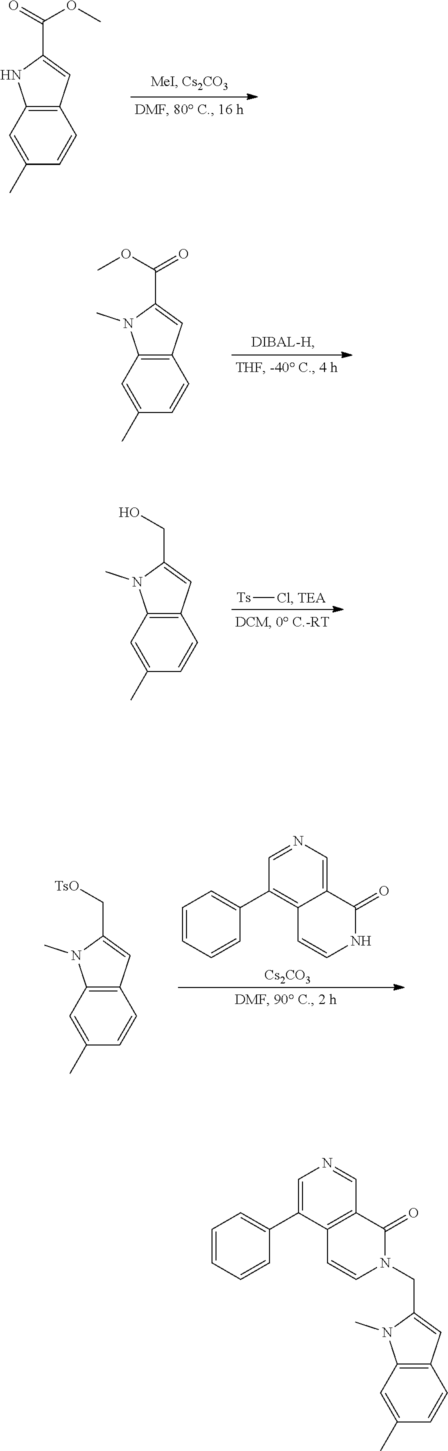

Step 1: The intermediate compound was prepared using the procedure described for example 60, step 1 with the following modification: the reaction was performed with methyl 6-methyl-1H-indole-2-carboxylate (0.3 g, 1.59 mmol) to give methyl 1,6-dimethyl-1H-indole-2-carboxylate (0.2 g, yield: 62%) as a white solid. MS: m/z: 204 [M+H]⁺

Step 2: The intermediate compound was prepared using the procedure described for example 60, step 2 with the following modification: the reaction was performed with of methyl 1,6-dimethyl-1H-indole-2-carboxylate(250 mg, 1.23 mmol) to afford (1,6-dimethyl-1H-indol-2-yl)methanol (100 mg, 571 μmol, yield: 46%) as a pale-yellow solid. MS: m/z: 158 [M-17]⁺

Step 3: The intermediate compound was prepared using the procedure described for example 60, step 3 with the following modification: the reaction was performed with (1,6-dimethyl-1H-indol-2-yl)methanol (100 mg, 571 μmol) to result in 8-(1,6-dimethyl-1H-indol-2-yl)methyl 4-methylbenzene-1-sulfonate (80 mg, yield: 79%) as pale brown semi-solid. MS: m/z: 330 [M+H]⁺

Step 4: The title compound was prepared using the procedure described for example 1, step 2 with the following modification: the reaction was performed with 5-phenyl-1,2-dihydro-2,7-naphthyridin-1-one and 8-(1,6-dimethyl-1H-indol-2-yl)methyl 4-methylbenzene-1-sulfonate to give 2-[(1,6-dimethyl-1H-indol-2-yl)methyl]-5-phenyl-1,2-dihydro-2,7-naphthyridin-1-one (yield: 10%) as a pale brown solid. ¹H NMR (400 MHz, DMSO-d₆): δ 9.45 (s, 1H), 8.70 (s, 1H), 7.74 (d, J=7.6 Hz, 1H), 7.56-7.48 (m, 6H), 7.36 (d, J=8.0 Hz, 1H), 7.22 (d, J=3.6 Hz, 1H), 6.85 (d, J=8.0 Hz, 1H), 6.50 (d, J=7.6 Hz, 1H), 5.44 (s, 2H), 3.72 (s, 3H), 2.40 (s, 3H). MS: m/z: 380 [M+H]⁺

### Synthetic Example 62: 2-((5-methylbenzofuran-2-yl)methyl)-5-phenyl-2,7-naphthyridin-1(2H)-one. (Compound No. 62)

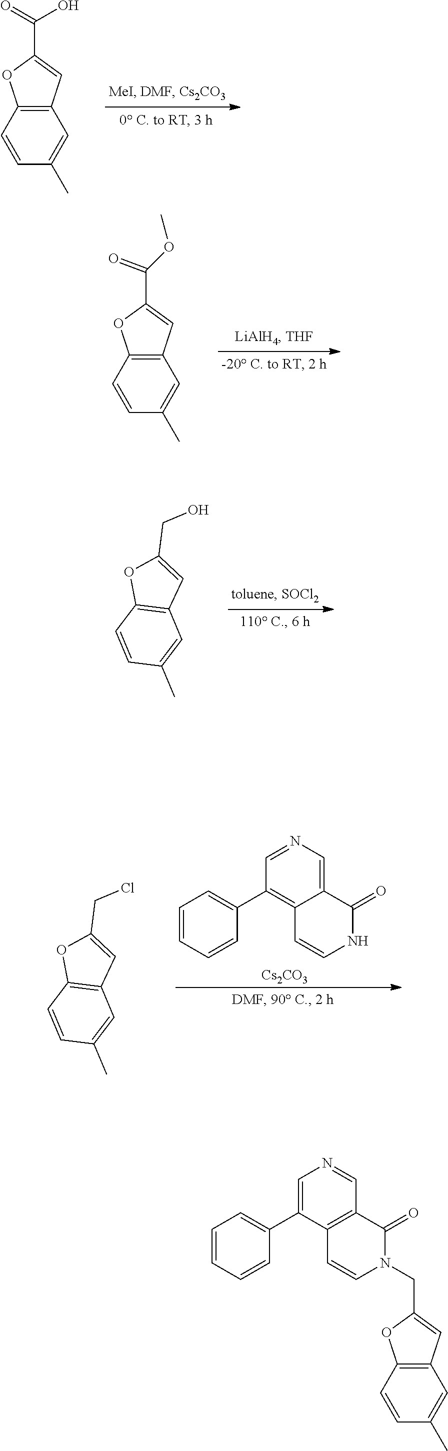

Step 1: Caesium carbonate (740 mg, 2.27 mmol) was added to a stirred solution of 5-methyl-1-benzofuran-2-carboxylic acid (0.2 g, 1.14 mmol) in DMF (4 mL) at 0° C., followed by addition of iodomethane (242 mg, 1.7 mmol). The reaction mixture was allowed to warm to room temperature, stirred for 3 h and diluted with ice-cold water (15 mL). The precipitated solid was filtered, dried to afford methyl 5-methyl-1-benzofuran-2-carboxylate (140 mg, yield: 65%) as a white solid. MS: m/z: 191 [M+H]⁺

Step 2: The intermediate compound was prepared using the procedure described for example 60, step 2 with the following modification: the reaction was performed with methyl 5-methyl-1-benzofuran-2-carboxylate (140 mg, 736 μmol) and purified by column chromatography (SiO₂, 2% MeOH/DCM) to obtain (5-methyl-1-benzofuran-2-yl)methanol (yield: 59%) as a white solid. MS: m/z: 163[M+H]⁺

Step 3: Thionyl chloride (0.5 mL) was added to a stirred solution of(5-methyl-1-benzofuran-2-yl)methanol (20 mg, 123 μmol) in toluene (3 mL) at 0° C. After 30 min, the reaction mixture was heated at 110° C. for 6 h, cooled to room temperature, neutralized with NaHCO₃ (10 mL) and extracted with EtOAc (30 mL×2). The combined organic layers were washed with brine (10 mL), dried over sodium sulphate, filtered and concentrated in vacuo to get 2-(chloromethyl)-5-methyl-1-benzofuran (60 mg, yield: 77%) as a pale brown solid. MS: m/z: 145 [M−Cl]⁺

Step 4: The title compound was prepared using the procedure described for example 1, step 2 with the following modification: the reaction was performed with 5-phenyl-1,2-dihydro-2,7-naphthyridin-1-one and 2-(chloromethyl)-5-methyl-1-benzofuran to give 2-[(5-methyl-1-benzofuran-2-yl)methyl]-5-phenyl-1,2-dihydro-2,7-naphthyridin-1-one (yield: 30%) as a pale brown gummy solid. ¹H NMR (400 MHz, DMSO-d₆): δ 9.42 (s, 1H), 8.70 (s, 1H), 7.89 (d, J=7.6 Hz, 1H), 7.58-7.54 (m, 2H), 7.52-7.49((m, 3H), 7.41 (s, 1H), 7.39 (s, 1H), 7.09 (dd, J=8.4, 1.2 Hz, 1H), 6.83 (d, J=0.4 Hz, 1H), 6.52 (dd, J=7.6, 0.8 Hz, 1H), 5.40 (s, 2H), 2.36 (s, 3H). MS: m/z: 367 [M+H]⁺

### Synthetic Example 63: Synthesis of 2-(benzo[d]oxazol-6-ylmethyl)-5-phenyl-2,7-naphthyridin-1(2H)-one. (Compound No. 63)

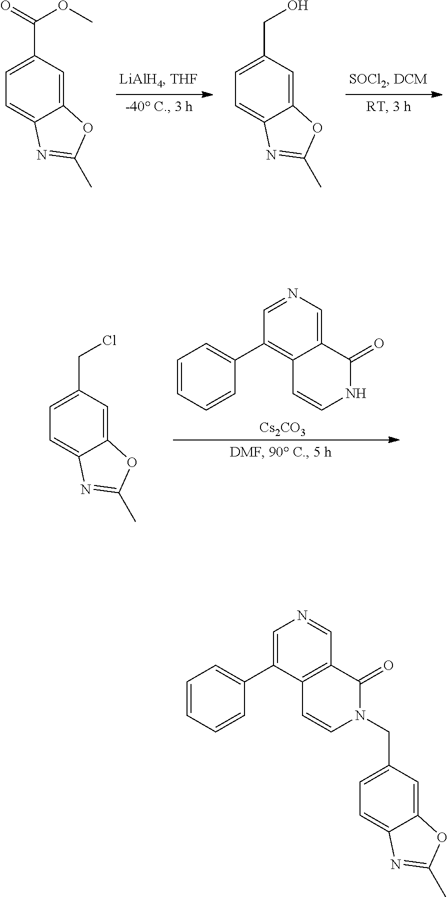

Step 1: The intermediate compound was prepared using the procedure described for example 60, step 2 with the following modification: the reaction was performed with methyl 2-methyl-1,3-benzoxazole-6-carboxylate (400 mg, 2.09 mmol) to give (2-methyl-1,3-benzoxazol-6-yl)methanol (200 mg, yield: 59%) as a pale-yellow solid. MS: m/z: 164 [M+H]⁺

Step 2: The intermediate compound was prepared using the procedure described for example 62, step 3 with the following modification: the reaction was performed with (2-methyl-1,3-benzoxazol-6-yl)methanol (120 mg, 735 μmol) to give 6-(chloromethyl)-2-methyl-1,3-benzoxazole (80 mg, yield: 60%) as red oily liquid. MS: m/z: 182 [M+H]⁺

Step 3: The title compound was prepared using the procedure described for example 1, step 2 with the following modification: the reaction was performed with 5-phenyl-1,2-dihydro-2,7-naphthyridin-1-one and 6-(chloromethyl)-2-methyl-1,3-benzoxazole to give 2-[(2-methyl-1,3-benzoxazol-6-yl)methyl]-5-phenyl-1,2-dihydro-2,7-naphthyridin-1-one (yield: 6%) as an off-white solid. ¹H NMR (400 MHz, DMSO-d₆): δ 9.41 (s, 1H), 8.67 (s, 1H), 7.89 (d, J=7.6 Hz, 1H), 7.69 (s, 1H), 7.54 (d, J=3.6 Hz, 1H), 7.51-7.47 (m, 5H), 7.36 (dd, J=8.0, 1.2 Hz, 1H), 6.47 (dd, J=7.6, 0.8 Hz, 1H), 5.32 (s, 2H), 2.59 (s, 3H). MS: m/z: 368 [M+H]⁺

### Synthetic Example 64: Synthesis of 2-((6-methyl-1H-indol-2-yl)methyl)-5-phenyl-2,7-naphthyridin-1(2H)-one. (Compound No. 64)

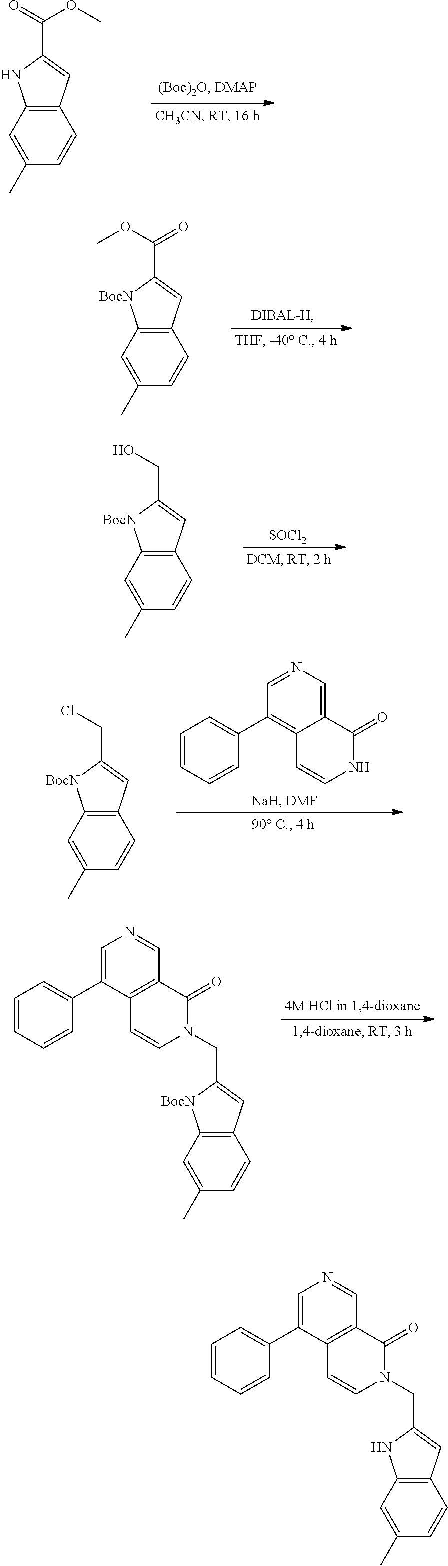

Step 1: Di-tert-butyl dicarbonate (138 mg, 634 μmol) was added to a stirred solution of methyl 6-methyl-1H-indole-2-carboxylate (100 mg, 529 μmol) in acetonitrile (5 mL), followed by the addition of DMAP (6.46 mg, 52.9 μmol). After 16 h at room temperature the reaction mixture was concentrated in vacuo and purified by column chromatography (SiO₂, 10% EtOAc/hexane) to afford 1-tert-butyl 2-methyl 6-methyl-1H-indole-1,2-dicarboxylate (80 mg, yield: 52%) as a white solid. MS: m/z: 290[M+H]⁺

Step 2: 25% DIBAL-H (1.96 mL, 0.0034 mmol) in toluene was added dropwise to a stirred solution of 1-(tert-butyl)₂-methyl 6-methyl-1H-indole-1,2-dicarboxylate (500 mg, 0.0017 mmol) in THF (10 vol) at −40° C. After 4 h, the reaction mixture was quenched with saturated ammonium chloride (10 mL) and extracted with ethyl acetate (50 mL×2). The organic layers were dried over sodium sulphate, filtered and concentrated in vacuo to give tert-butyl 2-(hydroxymethyl)-6-methyl-1H-indole-1-carboxylate (350 mg, yield: 77%) as a brown solid.

Step 3: The intermediate compound was prepared using the procedure described for example 62, step 3 with the following modification: the reaction was performed with tert-butyl 2-(hydroxymethyl)-6-methyl-1H-indole-1-carboxylate (350 mg, 0.0013 mmol) to give tert-butyl 2-(chloromethyl)-6-methyl-1H-indole-1-carboxylate (200 mg, yield: 53%) as a brown solid.

Step 4: 5-phenyl-1,2-dihydro-2,7-naphthyridin-1-one (75.1 mg, 338 μmol) was added to a suspension of 60% sodium hydride (16.2 mg, 676 μmol) in DMF (5 mL) at 0° C., followed by the addition of tert-butyl 2-(chloromethyl)-6-methyl-1H-indole-1-carboxylate (100 mg, 338 μmol). The reaction mixture was heated at 90° C. for 4 h, cooled to 0° C., diluted with ice-water (10 mL) and extracted with DCM (20 mL×2). The combined organic layers were washed with brine solution (10 mL), dried over sodium sulphate, filtered and concentrated in vacuo to give tert-butyl 6-methyl-2-[(1-oxo-5-phenyl-1,2-dihydro-2,7-naphthyridin-2-yl)methyl]-1H-indole-1-carboxylate (70 mg, yield: 10%) as a brown solid. MS: m/z: 466 [M+H]⁺

Step 5: The title compound was prepared using the procedure described for example 4, step 2 with the following modification: the reaction was performed with tert-butyl 6-methyl-2-[(1-oxo-5-phenyl-1,2-dihydro-2,7-naphthyridin-2-yl)methyl]-1H-indole-1-carboxylate (70.0 mg, 150 μmol) and purified by reverse phase prep HPLC (Column: YMC Triart C18 (250*20 mm)5μ; 0-95% ACN/H2O) to afford 2-[(6-methyl-1H-indol-2-yl)methyl]-5-phenyl-1,2-dihydro-2,7-naphthyridin-1-one (3.90 mg, yield: 7%) as an off white solid.

¹H NMR (400 MHz, DMSO-d₆): δ 10.96 (s, 1H), 9.43 (d, J=0.4 Hz, 1H), 8.67 (s, 1H), 7.78 (d, J=7.6 Hz, 1H), 7.57-7.47 (m, 5H), 7.33 (d, J=8.0 Hz, 1H), 7.13 (d, J=0.4 Hz, 1H), 6.79 (dd, J=8.0, 1.2 Hz, 1H), 6.47 (d, J=7.6 Hz, 1H), 6.31 (s, 1H), 5.32 (s, 2H), 2.35 (s, 3H). MS: m/z: 366 [M+H]⁺

### Synthetic Example 65: Synthesis of 2-((6-methylimidazo[1,2-a]pyrazin-2-yl)methyl)-5-phenyl-2,7-naphthyridin-1(2H)-one. (Compound No. 65)

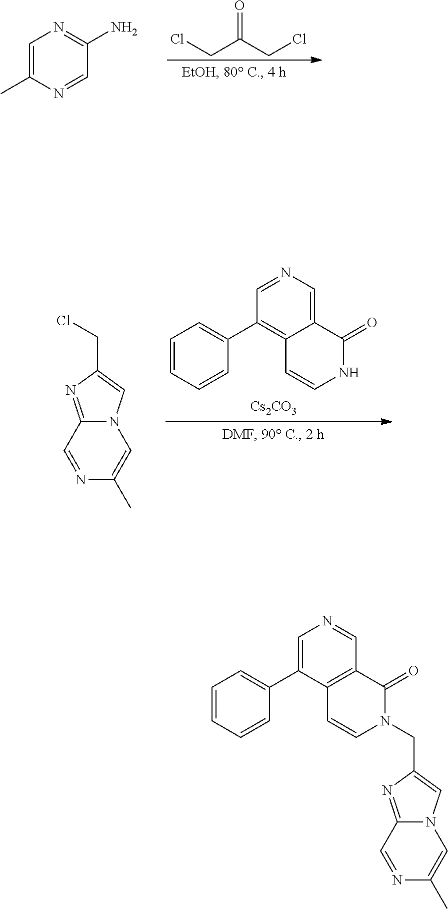

Step 1: The intermediate compound was prepared using the procedure described for example 1, step 1 with the following modification: the reaction was performed with 5-methylpyrazin-2-amine (50.0 mg, 458 μmol) to give 2-(chloromethyl)-6-methylimidazo[1,2-a]pyrazine (15.0 mg, yield: 1%) as a brown solid. MS: m/z: 182 [M+H]⁺

Step 2: The title compound was prepared using the procedure described for example 1, step 2 with the following modification: the reaction was performed with 5-phenyl-1,2-dihydro-2,7-naphthyridin-1-one and 2-(chloromethyl)-6-methylimidazo[1,2-a]pyrazine and purified by reverse phase prep-HPLC (Column: KINETEX, C18 (250×21.2 mm, 5μ); 0-95% CH₃CN/H₂O) to afford 2-({6-methylimidazo[1,2-a]pyrazin-2-yl}methyl)-5-phenyl-1,2-dihydro-2,7-naphthyridin-1-one (yield: 50%) as an off-white solid. ¹H NMR (400 MHz, DMSO-d₆) δ 9.39 (s, 1H), 8.90 (s, 1H), 8.67 (s, 1H), 8.36 (s, 1H), 8.00 (s, 1H), 7.87 (d, J=7.6 Hz, 1H), 7.60-7.54 (m, 2H), 7.53-7.49 (m, 3H), 6.48 (d, J=7.6 Hz, 1H), 5.37 (s, 2H), 2.39 (s, 3H). MS: m/z: 368 [M+H]⁺

### Synthetic Example 66: Synthesis of 4-methyl-2-((6-methylimidazo[1,2-a]pyridin-2-yl)methyl)-5-phenyl-2,7-naphthyridin-1(2H)-one. (Compound No. 66)

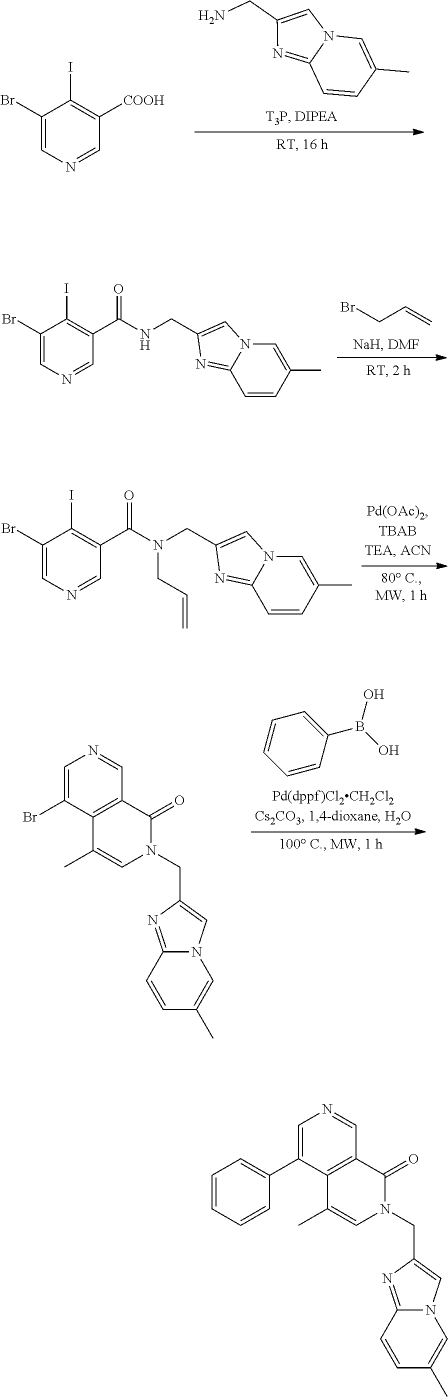

Step 1: To a stirred solution of 5-bromo-4-iodopyridine-3-carboxylic acid (0.2 g, 610 μmol) in DMF (3 mL) at room temperature was added DIPEA (236 mg, 1.83 mmol) followed by T3P (388 mg, 1.22 mmol) and after 15 min, 1-{6-methylimidazo[1,2-a]pyridin-2-yl}methanamine (118 mg, 732 μmol) was added to the reaction mixture. After 16 h, the reaction mixture was quenched with ice cold water (20 mL), and the resultant solid was filtered, dried under vacuum to give 5-bromo-4-iodo-N-({6-methylimidazo[1,2-a]pyridin-2-yl)}ethyl)pyridine-3-carboxamide(110 mg, yield: 39%) as an off white solid. MS: m/z: 472 [M+H]⁺

Step 2: 5-bromo-4-iodo-N-({6-methylimidazo[1,2-a]pyridin-2-yl}methyl)pyridine-3-carboxamide (0.1 g 212 μmol) was added to a suspension of 60% sodium hydride (15.3 mg 637 μmol) in DMF (4 mL) at 0° C. After 30 min, 3-bromoprop-1-ene (30.8 mg, 255 μmol) was added to the reaction mixture and stirred at room temperature for 2 h. The reaction was quenched with water (20 mL), and extracted with EtOAc (40 mL×2). The combined organic layers were dried over sodium sulphate, filtered, concentrated in vacuo and purified by flash chromatography (SiO2, 40% EtOAC/Hexane) to get 5-bromo-4-iodo-N-({6-methylimidazo[1,2-a]pyridin-2-yl}methyl)-N-(prop-2-en-1-yl)pyridine-3-carboxamide (68 mg, yield: 63%) as a gummy liquid. MS: m/z: 512 [M+H]⁺

Step 3: A stirred solution of 5-bromo-4-iodo-N-({6-methylimidazo[1,2-a]pyridin-2-yl}methyl)-N-(prop-2-en-1-yl)pyridine-3-carboxamide (30 mg, 58.7 μmol) in N,N-dimethylformamide (3 mL) was purged with argon for 10 min, followed by the addition of tetrabutylammonium bromide (18.9 mg, 58.7 μmol), triethylamine (5.94 mg, 58.7 μmol) and Pd(OAc)₂ (1.32 mg, 5.87 μmol). The reaction mixture was irradiated in a microwave reactor at 80° C. for 1 h, cooled to room temperature, diluted with ice cold water (3 mL), and extracted with 10% MeOH: DCM (60 mL×3). The combined organic layers were dried over sodium sulphate, filtered, and concentrated in vacuo to give 5-bromo-4-methyl-2-({6-methylimidazo[1,2-a]pyridin-2-yl}methyl)-1,2-dihydro-2,7-naphthyridin-1-one (24 mg, yield: 87%) as a gummy liquid. MS: m/z: 384 [M+H]⁺

Step 4: A stirred solution of 5-bromo-4-methyl-2-({6-methylimidazo[1,2-a]pyridin-2-yl}methyl)-1,2-dihydro-2,7-naphthyridin-1-one(20 mg, 52.2 μmol) in mixture of 1,4-dioxane and water (4:1)(2.5 mL) was purged with argon for 20 min, followed by the addition of phenylboronic acid (7.57 mg, 62.64 μmol), Cs₂CO₃ (56 mg, 358 μmol) and 1,1′-bis(triphenylphosphine)ferrocene]dichloropalladium(II), complex with dichloromethane (43.3 mg, 53.1 μmol). The reaction mixture was irradiated in a microwave reactor at 100° C. for 1 h, cooled to room temperature, and filtered through a celite pad. The filtrate was concentrated in vacuo and purified by reverse phase prep-HPLC (Column: YMC Triart C18 (250*20 mm)5μ; 0-95% CH₃CN/H2O) to give 4-methyl-2-({6-methylimidazo[1,2-a]pyridin-2-yl}methyl)-5-phenyl-1,2-dihydro-2,7-naphthyridin-1-one (1 mg, yield: 1%) as a brown solid. MS: m/z: 381 [M+H]⁺

### Synthetic Example 67: Synthesis of 2-((1-oxo-2,7-naphthyridin-2(1H)-yl)methyl)imidazo[1,2-a]pyridine-6-carbonitrile. (Compound No. 67)

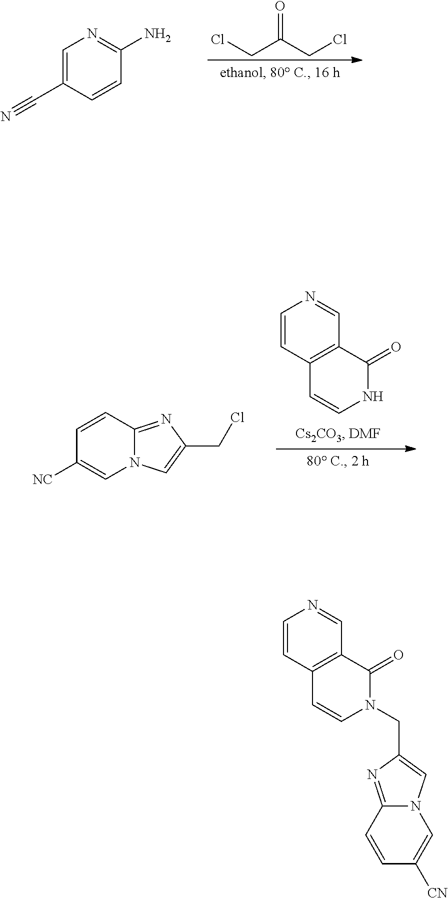 [SMILES: N#Cc1ccc(N)nc1 ; O=C(CCl)CCl ; N#Cc1ccc2nc(CCl)cn2c1 ; O=c1[nH]ccc2ccncc12 ; N#Cc1ccc2nc(Cn3ccc4ccncc4c3=O)cn2c1]

Step 1: The intermediate compound was prepared using the procedure described for example 1, step 1 with the following modification: the reaction was performed with 5-methylpyridin-2-amine (500 mg, 4.62 mmol) and the residue was purified by flash column chromatography (SiO₂, 2% MeOH/DCM) to give 2-(chloromethyl)-6-methylimidazo[1,2-a]pyridine (160 mg, yield: 19%) as an off-white solid. MS: m/z: 192[M+H]⁺

Step 2: The title compound was prepared using the procedure described for example 1, step 2 with the following modification: the reaction was performed with 1,2-dihydro-2,7-naphthyridin-1-one and 2-(chloromethyl)imidazo[1,2-a]pyridine-6-carbonitrile and purified by reverse phase Prep-HPLC (YMC C18(250*20 MM*5 u, 0-95% CH₃CN/H₂O) to afford 2-[(1-oxo-1,2-dihydro-2,7-naphthyridin-2-yl)methyl]imidazo[1,2-a]pyridine-6-carbonitrile (yield: 16%). ¹H NMR (400 MHz, DMSO-d₆): δ 9.34 (s, 1H), 9.28 (s, 1H), 8.71 (d, J=5.6 Hz, 1H), 7.98 (s, 1H), 7.87 (d, J=7.6 Hz, 1H), 7.66 (d, J=9.6 Hz, 1H), 7.60 (d, J=5.6 Hz, 1H), 7.48 (dd, J=1.6, 9.2 Hz, 1H), 6.68 (d, J=7.2 Hz, 1H), 5.34 (s, 2H). MS: m/z: 192[M+H]⁺

### Synthetic Example 68: Synthesis of 2-((6-(aminomethyl)imidazo[1,2-a]pyridin-2-yl)methyl)-2,7-naphthyridin-1(2H)-one. (Compound No. 68)

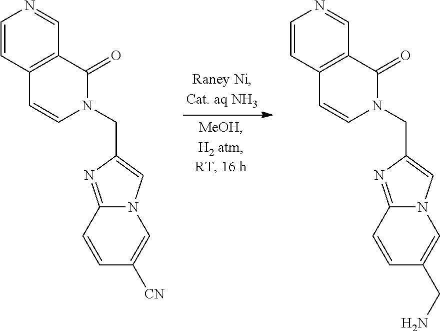 [SMILES: N#Cc1ccc2nc(Cn3ccc4ccncc4c3=O)cn2c1 ; NCc1ccc2nc(Cn3ccc4ccncc4c3=O)cn2c1]

To a stirred solution of 2-[(1-oxo-1,2-dihydro-2,7-naphthyridin-2-yl)methyl]imidazo[1,2-a]pyridine-6-carbonitrile (60 mg, 199 μmol) in methanol (10 mL) was added Raney Nickel (41.4 mg) followed by a catalytic amount of 25% aqueous ammonia (0.5 mL) under nitrogen atmosphere. The reaction mixture was hydrogenated under hydrogen balloon pressure for 16 h, passed through celite pad, and the filtrate was concentrated in vacuo and purified by reverse phase prep-HPLC (COLUMN: ACQUITY BEH C18(2.1*50*1.7 u), 0-95% CH₃CN/H₂O) to afford 2-{[6-(aminomethyl)imidazo[1,2-a]pyridin-2-yl]methyl}-1,2-dihydro-2,7-naphthyridin-1-one (6 mg, yield: 29%) as an off-white solid. ¹H NMR (400 MHz, DMSO-d₆(D₂O-exchange)): δ 9.35 (s, 1H), 8.71 (d, J=5.2 Hz, 1H), 8.48 (s, 1H), 7.89 (s, 1H), 7.85 (d, J=7.6 Hz, 1H), 7.60 (d, J=5.2 Hz, 1H), 7.50 (d, J=9.2 Hz, 1H), 7.30 (dd, J=0.8, 9.2 Hz, 1H), 6.68 (d, J=7.6 Hz, 1H), 5.29 (s, 2H), 3.87 (s, 2H). MS: m/z: 306 [M+H]⁺

### Synthetic Example 69: Synthesis of 2-((6-((benzylamino)methyl)imidazo[1,2-a]pyridin-2-yl)methyl)-2,7-naphthyridin-1(2H)-one. (Compound No. 69)

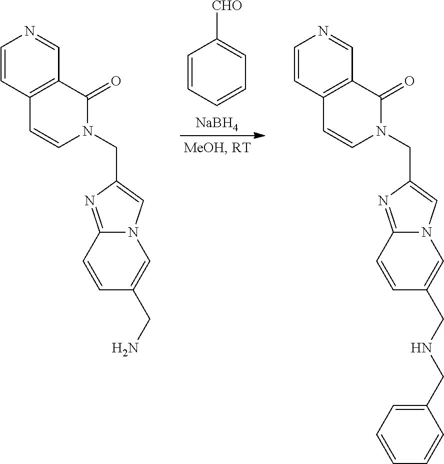

Benzaldehyde (18.6 mg, 179.2 μmol) was added to a stirred solution of 2-{[6-(aminomethyl)imidazo[1,2-a]pyridin-2-yl]methyl}-1,2-dihydro-2,7-naphthyridin-1-one (50 mg, 163 μmol) in methanol (5 mL) at room temperature and after 4 h, the reaction mixture was cooled to 0° C., and NaBH₄ (10.2 mg, 195.6 μmol) was added to the reaction mixture. After 1 h the reaction mixture was diluted with water (5 mL), and extracted with EtOAc (10 mL×2). The combined organic layers were dried over anhydrous sodium sulphate, filtered, concentrated in vacuo and purified by reverse phase prep-HPLC method (Column: YMC Triat C18 (250*21.2 mm)5μ, 0-95% CH₃CN/H₂O) to afford(2-({6-[(benzylamino)methyl]imidazo[1,2-a]pyridin-2-yl}methyl)-1,2-dihydro-2,7-naphthyridin-1-one (8 mg, yield: 12%) as an off-white solid. ¹H NMR (400 MHz, DMSO-d₆) δ 9.35 (s, 1H), 8.71 (d, J=5.4 Hz, 1H), 8.39 (d, J=1.8 Hz, 1H), 7.86 (s, 1H), 7.84 (d, J=1.6 Hz, 1H), 7.59 (dd, J=5.6, 0.8 Hz, 1H), 7.44 (d, J=9.6 Hz, 1H), 7.36-7.17 (m, 6H), 6.66 (d, J=7.6 Hz, 1H), 5.28 (s, 2H), 3.67 (s, 2H), 3.65 (s, 2H). MS: m/z: 396.2 [M+H]⁺

### Synthetic Example 70: Synthesis of 2-((6-(((cyclobutylmethyl)amino)methyl)imidazo[1,2-a]pyridin-2-yl)methyl)-2,7-naphthyridin-1(2H)-one. (Compound No. 70)

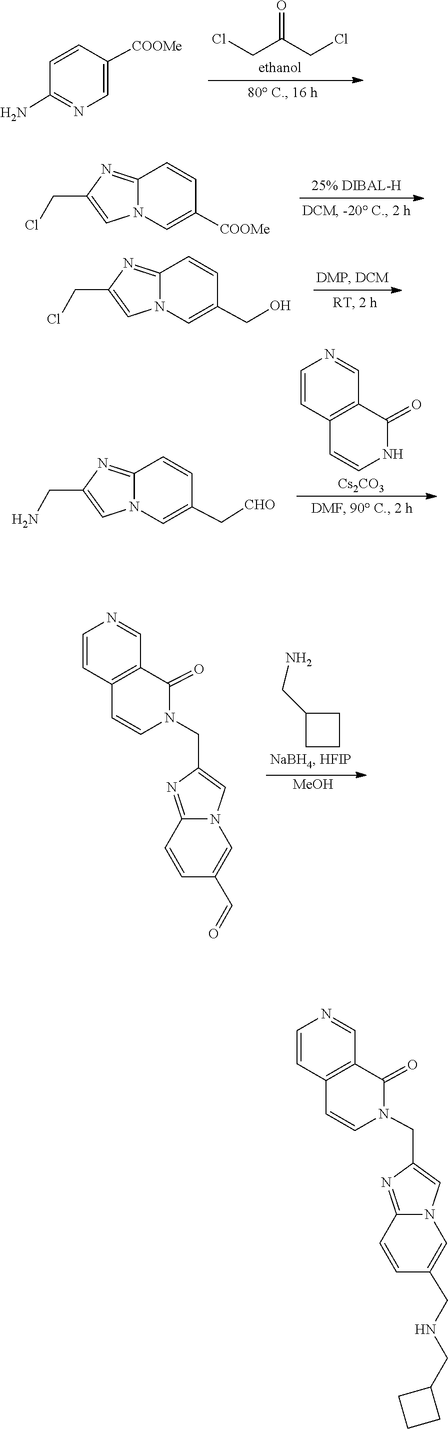

Step 1: The intermediate compound was prepared using the procedure described for example 1, step 1 with the following modification: the reaction was performed with methyl 6-aminopyridine-3-carboxylate (10 g 65.7 mmol) and purified by flash column chromatography (SiO₂, 20% EtOAc/hexane) to afford methyl 2-(chloromethyl)imidazo[1,2-a]pyridine-6-carboxylate(5.3 g, yield: 51%) as an off-white solid.

Step 2: 25% DIBAL-H (6.33 g, 44.5 mmol) was added dropwise to a stirred solution of methyl 2-(chloromethyl)imidazo[1,2-a]pyridine-6-carboxylate (5 g, 22.3 mmol) in dichloromethane (50 mL) at −20° C. After 2 h, the reaction mixture was quenched with saturated ammonium chloride solution (20 mL), and extracted with DCM (150 mL×2). The combined organic layers was dried over anhydrous sodium sulphate, filtered and concentrated in vacuo to give [2-(chloromethyl)imidazo[1,2-a]pyridin-6-yl]methanol (3.2 g yield: 65%) as a brown solid. MS: m/z: 197 [M+H]⁺

Step 3: Dess-Martin periodinane (1.62 g, 3.81 mmd) was added portion wise to a stirred solution of 2-(chloromethyl)-6-methylimidazo[1,2-a]pyridine (1.00 g, 5.54 mmd) in dichloromethane (10 mL) at 0° C. The reaction mixture was allowed to warm to stir at room temperature and stirred for 4 h. The reaction mixture was passed through a celite pad, the filtered was concentrated in vacuo and the resultant residue was purified by column chromatography (SiO2, 50% EtOAc/hexane) to afford 2-(chloromethyl)imidazo[1,2-a]pyridine-6-carbaldehyde (0.3 g, yield: 61%) as an off-white solid.

Step 4: Caesium carbonate (1.56 g 4.79 mmol) was added to a solution of 1,2-dihydro-2,7-naphthyridin-1-one (350 mg, 2.39 mmol) in DMF (5 mL), followed by addition of 2-(chloromethyl)imidazo[1,2-a]pyridine-6-carbaldehyde (466 mg, 2.39 mmol) at room temperature. The reaction mixture was heated at 80° C. for 4 h. The reaction mixture was cooled to room temperature, diluted with cold water (20 mL), the precipitated solid was filtered and dried to give 2-[(1-oxo-1,2-dihydro-2,7-naphthyridin-2-yl)methyl]imidazo[1,2-a]pyridine-6-carbaldehyde (250 mg, yield: 45%) as a pale brown solid. MS: m/z: 305.3 [M+H]⁺

Step 5: Cyclobutan-1-amine (27.8 mg, 329 μmol) was added to a stirred solution of 2-[(1-oxo-1,2-dihydro-2,7-naphthyridin-2-yl)methyl]imidazo[1,2-a]pyridine-6-carbaldehyde (0.1 g, 329 μmol) in HFIP (3 mL) at room temperature and stirred for 16 h. After 16 h, sodium borohydride (24.8 mg, 657 μmol) and catalytic amount of methanol (0.3 mL) were added and stirred for additional 2 h. The reaction mixture was diluted with ice cold water (15 mL), extracted with ethyl acetate (60 mL), concentrated in vacuo and the resultant residue was purified by reverse phase prep HPLC method (Column: GEMINI C18 2.0*50 mm, 3 μm: 0-95% ACN/H₂O) to give 1,2-[(6-{[(cyclobutylmethyl)amino]methyl}imidazo[1,2-a]pyridin-2-yl)methyl]-1,2-dihydro-2,7-naphthyridin-1-one(22 mg, yield: 18%) as an off-white solid. ¹H NMR (400 MHz, DMSO-d₆) δ 9.34 (s, 1H), 8.71 (d, J=5.6 Hz, 1H), 8.37 (s, 1H), 7.86 (s, 1H), 7.84 (d, J=3.6 Hz, 1H), 7.59 (d, J=6.0 Hz, 1H), 7.43 (d, J=9.2 Hz, 1H), 7.22 (dd, J=9.2, 1.2 Hz, 1H), 6.66 (d, J=7.2 Hz, 1H), 5.28 (s, 2H), 3.65 (s, 2H), 2.38-2.33 (m, 3H), 1.98-1.93 (m, 2H), 1.82-1.77 (m, 2H), 1.68-1.56 (m, 2H). MS: m/z: 374.48 [M+H]⁺

### Synthetic Example 71: Synthesis of 2-((6-(((cyclohexylmethyl)amino)methyl)imidazo[1,2-a]pyridin-2-yl)methyl)-2,7-naphthyridin-1(2H)-one. (Compound No. 71)

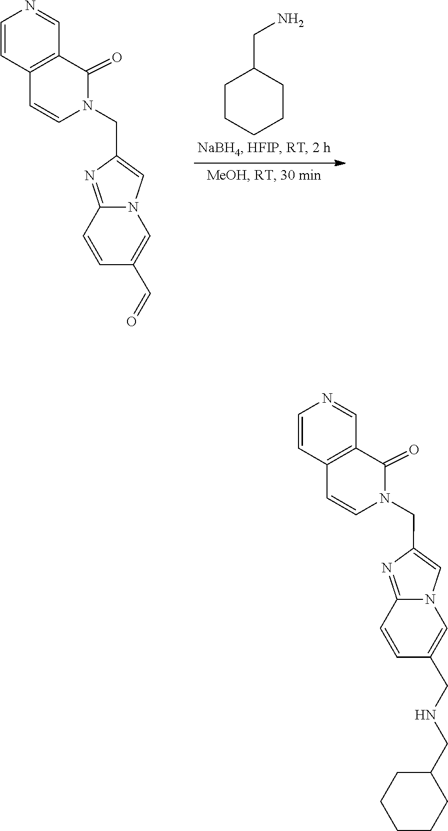 [SMILES: O=Cc1ccc2nc(Cn3ccc4ccncc4c3=O)cn2c1 ; NCC1CCCCC1 ; O=c1c2cnccc2ccn1Cc1cn2cc(CNCC3CCCCC3)ccc2n1]

The title compound was prepared using the procedure described for example 70, step 5 with the following modification: the reaction was performed with cyclohexyl methylamine to give 2-((6-(((cyclohexylmethyl)amino)methyl)imidazo[1,2-a]pyridin-2-yl)methyl)-2,7-naphthyridin-1(2H)-one (7 mg, yield: 11%) as an off-white solid. ¹H NMR (400 MHz, DMSO-d₆): δ9.34 (s, 1H), 8.71 (d, J=5.6 Hz, 1H), 8.37 (s, 1H), 8.16 (s, 1H), 7.87 (s, 1H), 7.84 (d, J=4.0 Hz, 1H), 7.59 (d, J=4.8 Hz, 1H), 7.43 (d, J=8.8 Hz, 1H), 7.22 (d, J=8.8 Hz, 1H), 6.67 (d, J=7.6 Hz, 1H), 6.53 (s, 1H), 6.29 (s, 1H), 5.28 (s, 2H), 3.66 (s, 2H), 1.73-1.70((m, 2H), 1.62-1.52 (m, 4H), 1.40-1.36 (m, 1H), 1.12-1.10 (m, 4H), 0.87-0.82 (m, 2H). MS: m/z: 402 [M+H]⁺

### Synthetic Example 72: Synthesis of 2-((6-((benzylamino)methyl)imidazo[1,2-a]pyridin-2-yl)methyl)-5-phenyl-2,7-naphthyridin-1(2H)-one. (Compound No. 72)

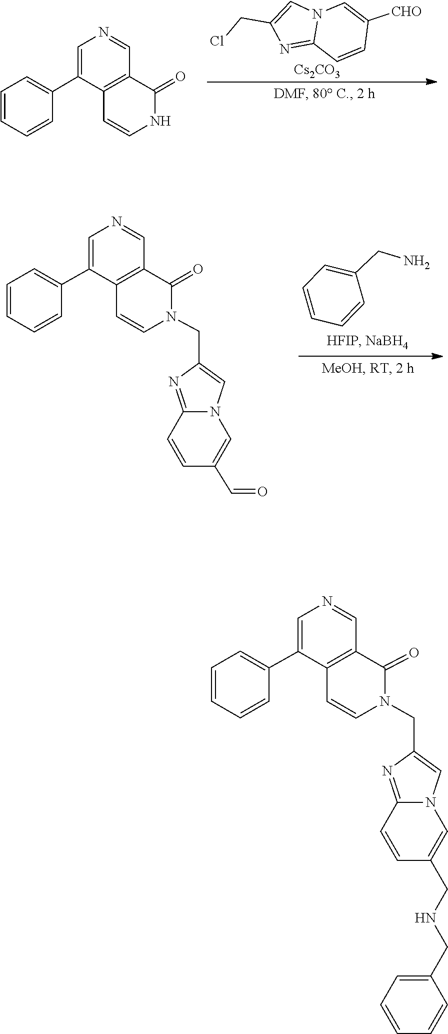

Step 1: The intermediate compound was prepared using the procedure described for example 70, step 4 with the following modification: the reaction was performed with 5-phenyl-1,2-dihydro-2,7-naphthyridin-1-one (40.0 mg, 180 μmol) to give 2-((1-oxo-5-phenyl-2,7-naphthyridin-2(1H)-yl)methyl)imidazo[1,2-a]pyridine-6-carbaldehyde(yield: 88%) as pale brown solid.

MS: m/z: 381 [M+H]⁺

Step 2: The title compound was prepared using the procedure described for example 70, step 5 with the following modification: the reaction was performed with 2-[(1-oxo-5-phenyl-1,2-dihydro-2,7-naphthyridin-2-yl)methyl]imidazo[1,2-a]pyridine-6-carbaldehyde and benzyl amine to give 2-({6-[(benzylamino)methyl]imidazo[1,2-a]pyridin-2-yl}methyl)-5-phenyl-1,2-dihydro-2,7-naphthyridin-1-one (yield: 5%) as an off-white solid. ¹H NMR (400 MHz, DMSO-d₆): δ 9.39 (d, J=0.8 Hz, 1H), 8.66 (s, 1H), 8.39 (s, 1H), 7.85 (d, J=7.2 Hz, 2H), 7.60-7.40 (m, 6H), 7.37-7.17 (m, 6H), 6.47 (d, J=7.6 Hz, 1H), 5.29 (s, 2H), 3.66 (s, 3H), 3.64 (s, 1H). MS: m/z: 472 [M+H]⁺

### Synthetic Example 73: 2-((6-((neopentylamino)methyl)imidazo[1,2-a]pyridin-2-yl)methyl)-5-phenyl-2,7-naphthyridin-1(2H)-one. (Compound No. 73)

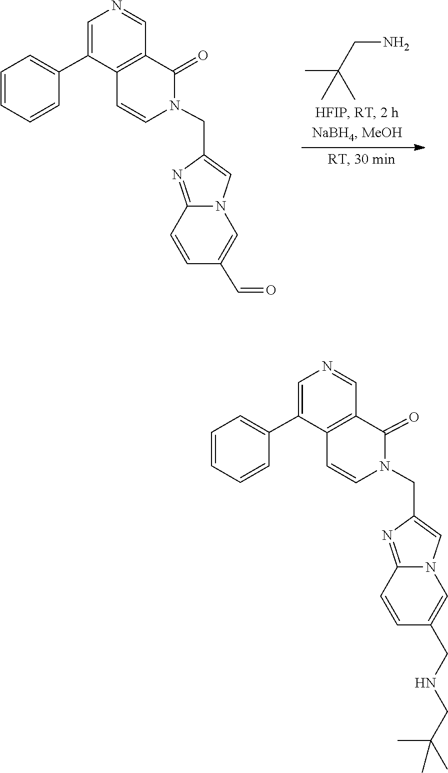

The title compound was prepared using the procedure described for example 72, step 2 with the following modification: the reaction was performed with 2,2-dimethylpropan-1-amine and purified by reverse phase prep HPLC method (Column: YMC TRIATC18(250*20 mm)5 μ, 0-95% CH₃CN/H₂O) to afford 2-[(6-{[(2,2-dimethylpropyl)amino]methyl}imidazo[1,2-a]pyridin-2-yl)methyl]-5-phenyl-1,2-dihydro-2,7-naphthyridin-1-one (yield: 23%) as an off-white solid. ¹H NMR (400 MHz, DMSO-d₆): δ 9.39 (s, 1H), 8.66 (s, 1H), 8.36 (s, 1H), 7.86 (s, 1H), 7.84 (d, J=1.2 Hz, 2H), 7.57-7.53 (m, 2H), 7.52-7.49 (m, 3H), 7.43 (d, J=9.2 Hz, 1H), 7.24 (dd, J=9.2, 1.6 Hz, 1H), 6.47 (dd, J=7.6, 0.8 Hz, 1H), 5.29 (s, 2H), 3.66 (s, 2H), 2.19 (s, 2H), 0.84 (s, 9H). MS: m/z: 452 [M+H]⁺

### Synthetic Example 74: Synthesis of 2-((6-((((1-hydroxycyclobutyl)methyl)amino)methyl)imidazo[1,2-a]pyridin-2-yl)methyl)-5-phenyl-2,7-naphthyridin-1(2H)-one. (Compound No. 74)

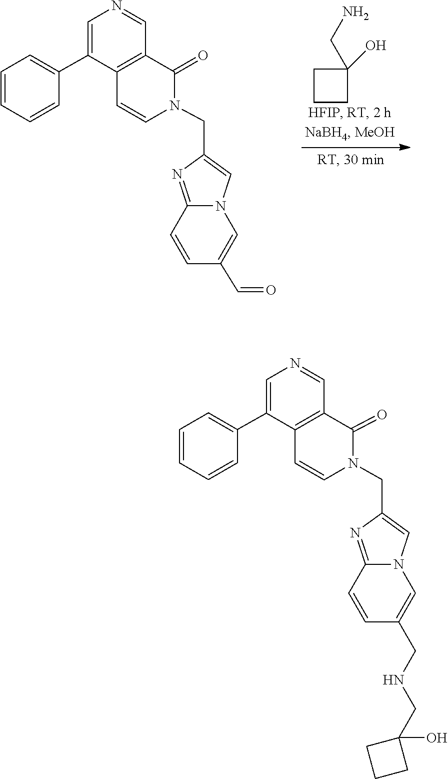 [SMILES: O=Cc1ccc2nc(Cn3ccc4c(-c5ccccc5)cncc4c3=O)cn2c1 ; NCC1(O)CCC1 ; O=c1c2cncc(-c3ccccc3)c2ccn1Cc1cn2cc(CNCC3(O)CCC3)ccc2n1]

The title compound was prepared using the procedure described for example 72, step 2 with the following modification: the reaction was performed with 1-hydroxycyclobutyl methylamine to give 2-{[6-({[(1-hydroxycyclobutyl)methyl]amino}methyl)imidazo[1,2-a]pyridin-2-yl]methyl}-5-phenyl-1,2-dihydro-2,7-naphthyridin-1-one (3.50 mg, yield: 7%) as a gummy solid. ¹H NMR (400 MHz, DMSO-d₆): δ 9.39 (s, 1H), 8.66 (s, 1H), 8.39 (s, 1H), 8.25 (s, 1H), 7.86-7.84 (m, 2H), 7.57-7.48 (m, 4H), 7.43 (d, J=9.2 Hz, 1H), 7.24 (dd, J=9.2, 1.6 Hz, 1H), 6.47 (d, J=7.2 Hz, 1H), 5.29 (s, 2H), 3.71 (s, 2H), 2.61-2.59 (m, 2H), 1.97-1.86 (m, 4H), 1.61-1.57 (m, 1H), 1.41-1.34 (m, 1H). MS: m/z: 466 [M+H]⁺

### Synthetic Example 75: 2-((6-((((1-fluorocyclobutyl)methyl)amino)methyl)imidazo[1,2-a]pyridin-2-yl)methyl)-5-phenyl-2,7-naphthyridin-1(2H)-one. (Compound No. 75)

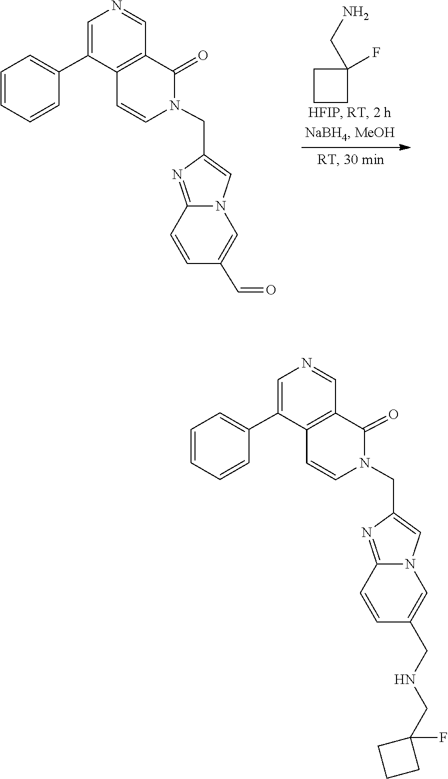 [SMILES: O=Cc1ccc2nc(Cn3ccc4c(-c5ccccc5)cncc4c3=O)cn2c1 ; NCC1(F)CCC1 ; O=c1c2cncc(-c3ccccc3)c2ccn1Cc1cn2cc(CNCC3(F)CCC3)ccc2n1]

The title compound was prepared using the procedure described for example 72, step 2 with the following modification: the reaction was performed with 1-fluorocyclobutyl methylamine to give 2-{[6-(([(1-fluorocyclobutyl)methyl]amino}methyl)imidazo[1,2-a]pyridin-2-yl]methyl)-5-phenyl-1,2-dihydro-2,7-naphthyridin-1-one (6.20 mg, yield: 10%) as an off-white solid. ¹H NMR (400 MHz, DMSO-d₆): δ 9.39 (s, 1H), 8.66 (s, 1H), 8.38 (s, 1H), 8.18 (s, 1H), 7.86-7.84 (m, 2H), 7.58-7.47 (m, 4H), 7.44 (d, J=9.2 Hz, 1H), 7.25 (dd, J=9.2, 1.6 Hz, 1H), 6.47 (dd, J=7.6, 0.8 Hz, 1H), 5.29 (s, 2H), 3.71 (s, 2H), 2.73 (s, 1H), 2.66 (s, 1H), 2.16-2.03 (m, 4H), 1.73-1.67 (m, 1H), 1.42-1.34 (m, 1H). MS: m/z: 468 [M+H]⁺

### Synthetic Example 76: 2-((6-(((cyclopentylmethyl)amino)methyl)imidazo[1,2-a]pyridin-2-yl)methyl)-5-phenyl-2,7-naphthyridin-1(2H)-one. (Compound No. 76)

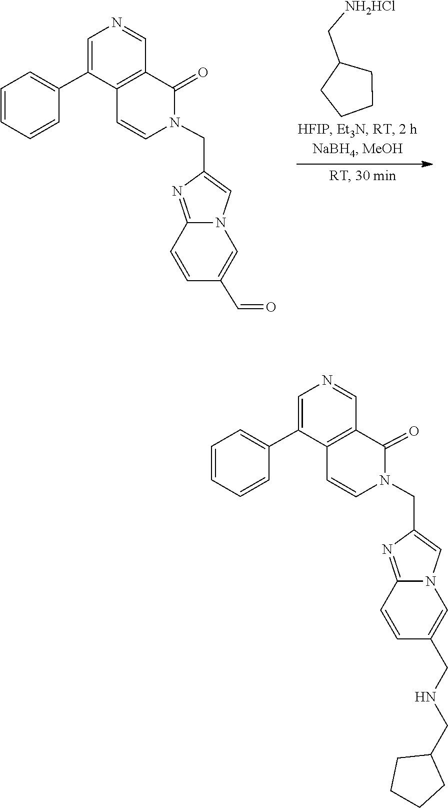

The title compound was prepared using the procedure described for example 72, step 2 with the following modification: the reaction was performed with cyclopentyl methylamine to give 2-[(6-{[(cyclopentylmethyl)amino]methyl}imidazo[1,2-a]pyridin-2-yl)methyl]-5-phenyl-1,2-dihydro-2,7-naphthyridin-1-one (yield: 24%) as an off-white solid. ¹H NMR (400 MHz, DMSO-d₆): δ 9.39 (d, J=0.8 Hz, 1H), 8.66 (s, 1H), 8.38 (s, 1H), 7.86 (s, 1H), 7.84 (s., 1H), 7.58-7.54 (m, 2H), 7.52-7.48((m, 3H), 7.43 (d, J=9.2 Hz, 1H), 7.23 (dd, J=9.2, 1.6 Hz, 1H), 6.47 (dd, J=7.6, 0.8 Hz, 1H), 5.29 (s, 2H), 3.67 (s, 2H), 2.39 (d, J=7.2 Hz, 2H), 1.95 (hept, J=7.6 Hz, 1H), 1.69-1.62 (m, 2H), 1.53-1.39 (m, 4H), 1.19-1.08 (m, 2H). MS: m/z: 464 [M+H]⁺

### Synthetic Example 77: 2-((6-(((cyclohexylmethyl)amino)methyl)imidazo[1,2-a]pyridin-2-yl)methyl)-5-phenyl-2,7-naphthyridin-1(2H)-one. (Compound No. 77)

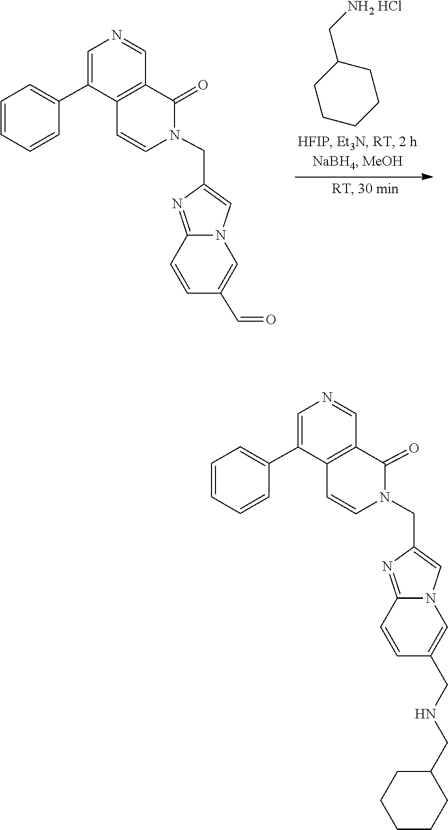

The title compound was prepared using the procedure described for example 72, step 2 with the following modification: the reaction was performed with cyclohexyl methylamine to give 2-[(6-{[(cyclohexylmethyl)amino]methyl}imidazo[1,2-a]pyridin-2-yl)methyl]-5-phenyl-1,2-dihydro-2,7-naphthyridin-1-one (yield: 35%) as an off-white solid. ¹H NMR (400 MHz, DMSO-d₆): δ 9.39 (s, 1H), 8.66 (s, 1H), 8.36 (d, J=10.3 Hz, 2H), 7.89-7.82 (m, 2H), 7.60-7.46 (m, 4H), 7.42 (d, J=9.2 Hz, 1H), 7.22 (dd, J=9.2, 1.6 Hz, 1H), 6.46 (d, J=7.6 Hz, 1H), 5.29 (s, 2H), 3.64 (s, 2H), 2.29 (d, J=6.8 Hz, 2H), 1.75-1.70 (m, 2H), 1.64-1.61 (m, 2H), 1.39-1.35 (m, 1H), 1.18-1.10 (m, 4H), 0.88-0.85 (m, 2H). MS: m/z: 478 [M+H]⁺

### Synthetic Example 78: 5-phenyl-2-((6-(((1-phenylcyclopropyl)amino)methyl)imidazo[1,2-a]pyridin-2-yl)methyl)-2,7-naphthyridin-1(2H)-one. (Compound No. 78)

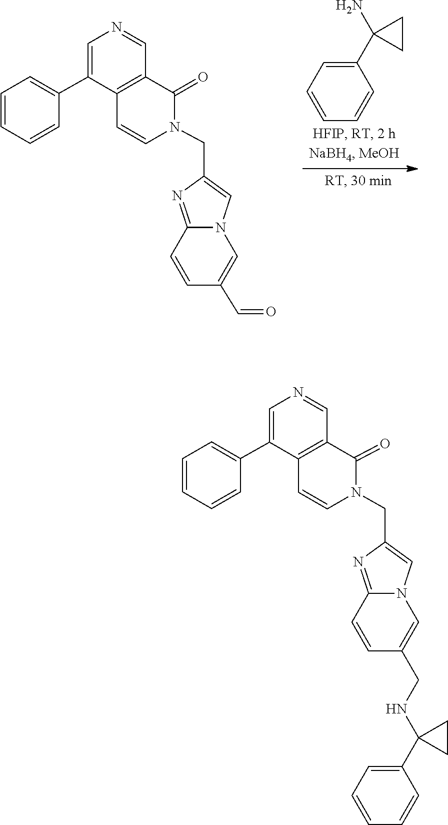 [SMILES: O=Cc1ccc2nc(Cn3ccc4c(-c5ccccc5)cncc4c3=O)cn2c1 ; NC1(c2ccccc2)CC1 ; O=c1c2cncc(-c3ccccc3)c2ccn1Cc1cn2cc(CNC3(c4ccccc4)CC3)ccc2n1]

The title compound was prepared using the procedure described for example 72, step 2 with the following modification: the reaction was performed with 1-phenylcyclopropylamine to give 5-phenyl-2-[(6-{[(1-phenylcyclopropyl)amino]methyl}imidazo[1,2-a]pyridin-2-yl)methyl]-1,2-dihydro-2,7-naphthyridin-1-one (yield: 12%) as an off-white solid. ¹H NMR (400 MHz, DMSO-d₆): δ 9.39 (d, J=0.4 Hz, 1H), 8.66 (s, 1H), 8.34 (s, 1H), 7.85-7.83 (m, 2H), 7.58-7.55 (m, 2H), 7.54-7.48((m, 3H), 7.40 (s, 1H), 7.37 (dd, J=8.0, 1.2 Hz, 2H), 7.32-7.28 (m, 2H), 7.19-7.15 (m, 2H), 6.46 (dd, J=7.6, 0.8 Hz, 1H), 5.29 (s, 2H), 3.54 (s, 2H), 0.96-0.94 (m, 2H), 0.88-0.85 (m, 2H). MS: m/z: 498 [M+H]⁺

### Synthetic Example 79: 2-((6-((((3-fluorocyclobutyl)methyl)amino)methyl)imidazo[1,2-a]pyridin-2-yl)methyl)-5-phenyl-2,7-naphthyridin-1(2H)-one. (Compound No. 79)

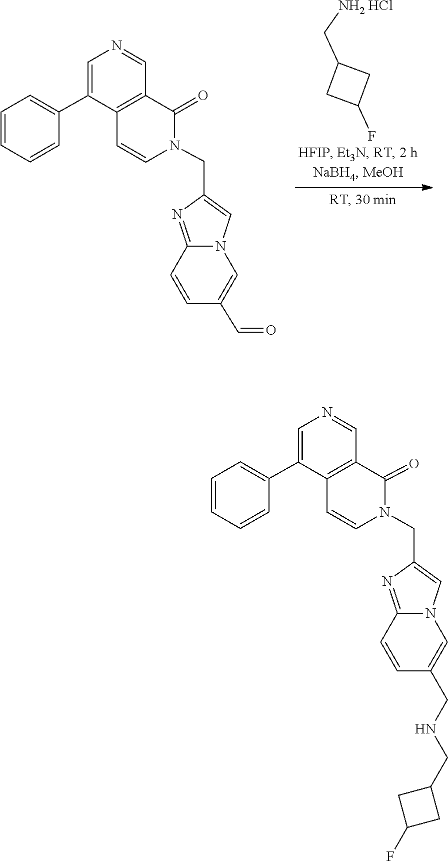

The title compound was prepared using the procedure described for example 72, step 2 with the following modification: the reaction was performed with 3-fluorocyclobutyl methanamine hydrochloride to give 2-{[6-({[(3-fluorocyclobutyl)methyl]amino}methyl)imidazo[1,2-a]pyridin-2-yl]methyl}-5-phenyl-1,2-dihydro-2,7-naphthyridin-1-one (yield: 42%) as an off-white solid. 1H NMR (400 MHz, DMSO-d₆): δ 9.39 (s, 1H), 8.66 (s, 1H), 8.38 (s, 1H), 7.86 (s, 1H), 7.84 (s, 1H), 7.58-7.54 (m, 2H), 7.52-7.48 (m, 3H), 7.44 (d, J=9.2 Hz, 1H), 7.23 (dd, J=9.2, 1.2 Hz, 1H), 6.47 (d, J=7.6 Hz, 1H), 5.29 (s, 2H), 3.68 (s, 2H), 2.51-2.50 (m, 3H), 2.41-2.32 (m, 1H), 2.16-2.08 (m, 4H). MS: m/z: 468 [M+H]⁺

### Synthetic Example 80: 5-phenyl-2-((6-(((1-phenylcyclobutyl) amino)methyl)imidazo[1,2-a]pyridin-2-yl)methyl)-2,7-naphthyridin-1(2H)-one. (Compound No. 80)

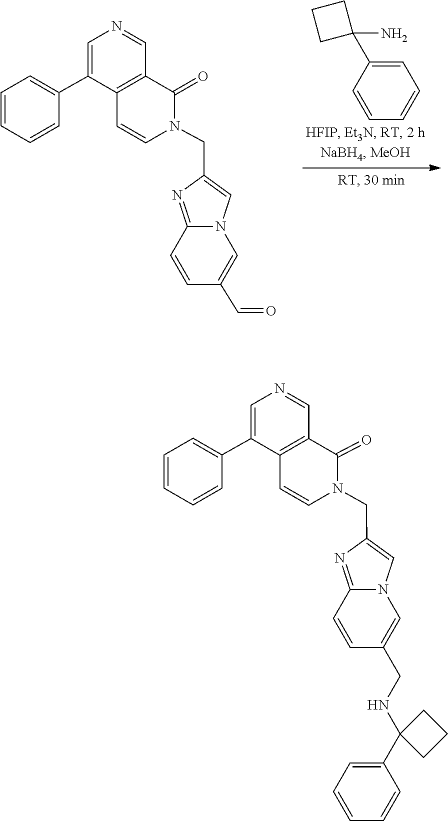 [SMILES: O=Cc1ccc2nc(Cn3ccc4c(-c5ccccc5)cncc4c3=O)cn2c1 ; NC1(c2ccccc2)CCC1 ; O=c1c2cncc(-c3ccccc3)c2ccn1Cc1cn2cc(CNC3(c4ccccc4)CCC3)ccc2n1]

The title compound was prepared using the procedure described for example 72, step 2 with the following modification: the reaction was performed with 1-phenylcyclobutylamine to give 5-phenyl-2-[(6-{[(1-phenylcyclobutyl)amino]methyl}imidazo[1,2-a]pyridin-2-yl)methyl]-1,2-dihydro-2,7-naphthyridin-1-one (yield: 14%) as an off-white solid. ¹H NMR (400 MHz, DMSO-d₆): δ 9.39 (s, 1H), 8.66 (s, 1H), 8.32 (s, 1H), 7.83 (t, J=6.0 Hz, 2H), 7.57-7.50 (m, 7H), 7.48-7.32 (m, 3H), 7.22-7.19 (m, 1H), 7.12 (dd, J=9.2, 1.6 Hz, 1H), 6.46 (d, J=7.2 Hz, 1H), 5.28 (s, 2H), 3.22 (s, 2H), 2.33-2.26 (m, 2H), 2.22-2.15 (m, 2H), 2.05-1.95 (m, 1H), 1.72-1.61 (m, 1H). MS: m/z: 512 [M+H]⁺

### Synthetic Example 81: 5-phenyl-2-((6-(((2-phenylpropan-2-yl)amino)methyl)imidazo[1,2-a]pyridin-2-yl)methyl)-2,7-naphthyridin-1(2H)-one. (Compound No. 81)

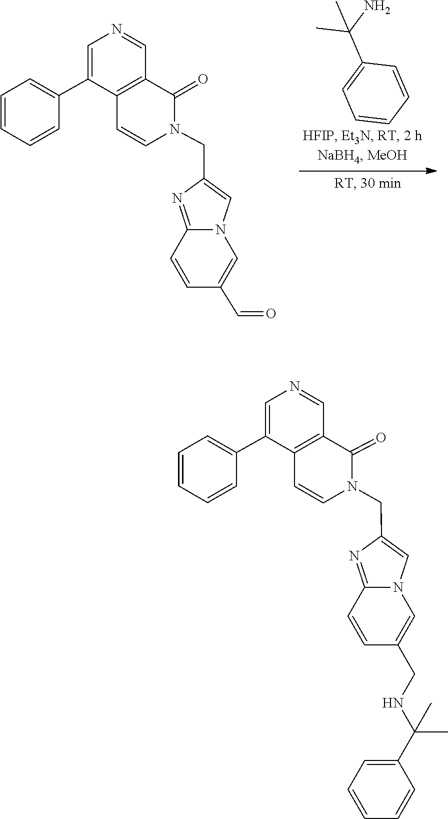

The title compound was prepared using the procedure described for example 72, step 2 with the following modification: the reaction was performed with 2-phenylpropan-2-amine to give 5-phenyl-2-[(6-{[(2-phenylpropan-2-yl)amino]methyl}imidazo[1,2-a]pyridin-2-yl)methyl]-1,2-dihydro-2,7-naphthyridin-1-one (yield:15%) as an off-white solid. ¹H NMR (400 MHz, DMSO-d₆): δ 9.39 (s, 1H), 9.21 (s, 1H), 8.67 (s, 1H), 8.45 (s, 1H), 7.96 (s, 1H), 7.84 (d, J=7.6 Hz, 1H), 7.61-7.53 (m, 2H), 7.57-7.46 (m, 8H), 7.18 (d, J=9.2 Hz, 1H), 6.47 (d, J=7.6 Hz, 1H), 5.31 (s, 2H), 3.80 (s, 2H), 1.76 (s, 3H), 1.45 (s, 3H). MS: m/z: 500 [M+H]⁺

### Synthetic Example 82: 2-((6-(((2-hydroxy-1-phenylethyl)amino)methyl)imidazo[1,2-a]pyridin-2-yl)methyl)-5-phenyl-2,7-naphthyridin-1(2H)-one. (Compound No. 82)

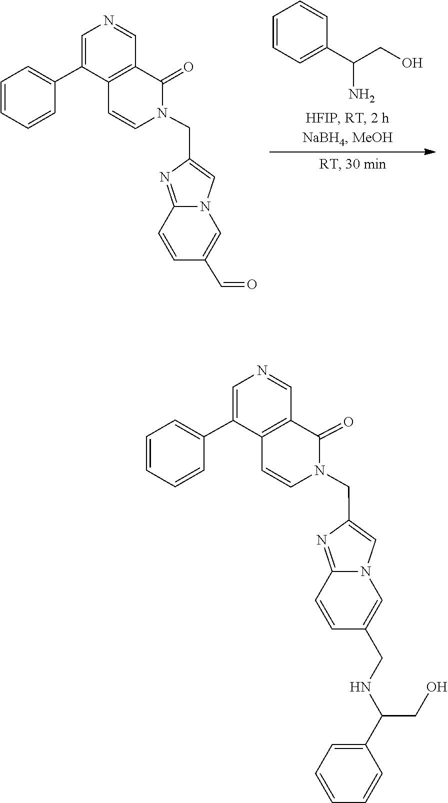 [SMILES: O=Cc1ccc2nc(Cn3ccc4c(-c5ccccc5)cncc4c3=O)cn2c1 ; NC(CO)c1ccccc1 ; O=c1c2cncc(-c3ccccc3)c2ccn1Cc1cn2cc(CNC(CO)c3ccccc3)ccc2n1]

The title compound was prepared using the procedure described for example 72, step 2 with the following modification: the reaction was performed with 2-hydroxy-1-phenylethylamine to give 2-[(6-{[(2-hydroxy-1-phenylethyl)amino]methyl}imidazo[1,2-a]pyridin-2-yl)methyl]-5-phenyl-1,2-dihydro-2,7-naphthyridin-1-one (yield: 15%) as an off-white solid. ¹H NMR (400 MHz, DMSO-d₆): δ 9.39 (d, J=0.8 Hz, 1H), 8.66 (s, 1H), 8.33 (s, 1H), 7.86 (s, 1H), 7.84 (d, J=0.8 Hz, 1H), 7.57-7.51 (m, 2H), 7.50-7.48 (m, 3H), 7.42 (d, J=9.2 Hz, 1H), 7.37-7.33 (m, 2H), 7.35-7.29((m, 2H), 7.27-7.23 (m, 1H), 7.18 (dd, J=9.2, 1.6 Hz, 1H), 6.47 (dd, J=7.6, 0.8 Hz, 1H), 5.29 (s, 2H), 4.82 (t, J=5.6 Hz, 1H), 3.66 (dd, J=8.0, 4.4 Hz, 1H), 3.56 (d, J=14.0 Hz, 1H), 3.45-3.32((m, 2H). MS: m/z: 502.4 [M+H]⁺

### Synthetic Example 83: 2-((6-(((cyclobutylmethyl)amino)methyl)imidazo[1,2-a]pyridin-2-yl)methyl)-5-phenyl-2,7-naphthyridin-1(2H)-one. (Compound No. 83)

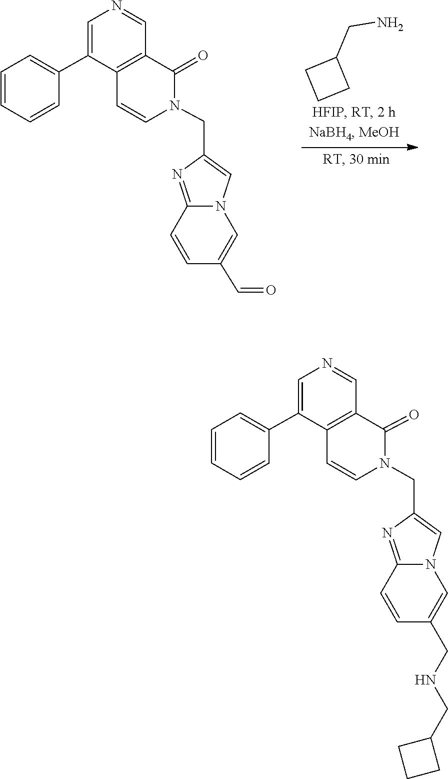 [SMILES: O=Cc1ccc2nc(Cn3ccc4c(-c5ccccc5)cncc4c3=O)cn2c1 ; NCC1CCC1 ; O=c1c2cncc(-c3ccccc3)c2ccn1Cc1cn2cc(CNCC3CCC3)ccc2n1]

The title compound was prepared using the procedure described for example 72, step 2 with the following modification: the reaction was performed with cyclobutylmethylamine to give 2-[(6-{[(cyclobutylmethyl)amino]methyl}imidazo[1,2-a]pyridin-2-yl)methyl]-5-phenyl-1,2-dihydro-2,7-naphthyridin-1-one (yield: 10%) obtained as an off-white solid. ¹H NMR (400 MHz, DMSO-d₆): δ 9.39 (d, J=0.4 Hz, 1H), 8.66 (s, 1H), 8.36 (s, 1H), 7.86-7.84 (m, 2H), 7.58-7.48 (m, 5H), 7.42 (d, J=9.2 Hz, 1H), 7.22 (dd, J=9.2, 1.6 Hz, 1H), 6.47 (d, J=7.6 Hz, 1H), 5.29 (s, 2H), 3.62 (s, 2H), 2.49 (d, J=5.2 Hz, 2H), 2.46-2.37 (m, 1H), 1.96-1.94 (m, 2H), 1.79-1.60 (m, 2H), 1.59-1.55 (m, 2H). MS: m/z: 450 [M+H]⁺

### Synthetic Example 84: 2-((6-(((4-chlorobenzyl)amino)methyl)imidazo[1,2-a]pyridin-2-yl)methyl)-5-phenyl-2,7-naphthyridin-1(2H)-one. (Compound No. 84)

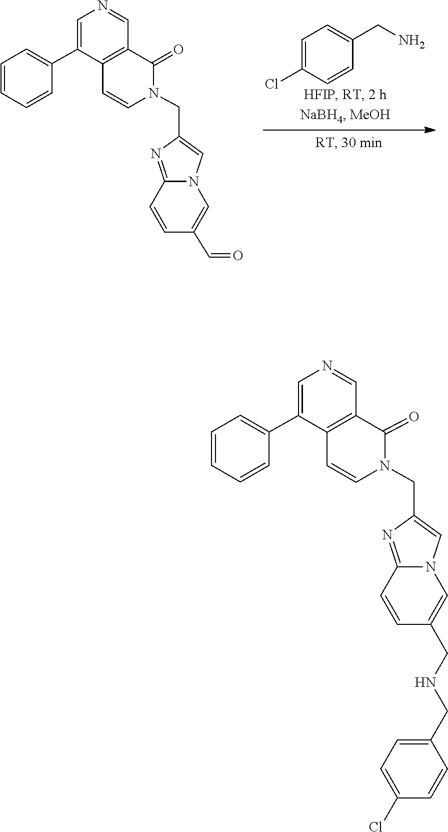 [SMILES: O=Cc1ccc2nc(Cn3ccc4c(-c5ccccc5)cncc4c3=O)cn2c1 ; NCc1ccc(Cl)cc1 ; O=c1c2cncc(-c3ccccc3)c2ccn1Cc1cn2cc(CNCc3ccc(Cl)cc3)ccc2n1]

The title compound was prepared using the procedure described for example 72, step 2 with the following modification: the reaction was performed with 4-chlorophenylmethylamine to give 2-{[6-({[(4-chlorophenyl)methyl]amino)methyl}imidazo[1,2-a]pyridin-2-yl]methyl}-5-phenyl-1,2-dihydro-2,7-naphthyridin-1-one (yield: 14%) as an off-white solid. ¹H NMR (400 MHz, DMSO-d₆): δ 9.39 (d, J=0.8 Hz, 1H), 8.66 (s, 1H), 8.38 (s, 1H), 7.86-7.84 (m, 2H), 7.57-7.48 (m, 5H), 7.44 (d, J=9.2 Hz, 1H), 7.36 (s, 4H), 7.24 (dd, J=9.2, 1.6 Hz, 1H), 6.47 (dd, J=7.6, 0.8 Hz, 1H), 5.29 (s, 2H), 3.65 (s, 2H), 3.62 (s, 2H). MS: m/z: 507 [M+H]⁺

### Synthetic Example 85: 2-((6-((((4-methylcyclohexyl)methyl)amino)methyl)imidazo[1,2-a]pyridin-2-yl)methyl)-5-phenyl-2,7-naphthyridin-1(2H)-one. (Compound No. 85)

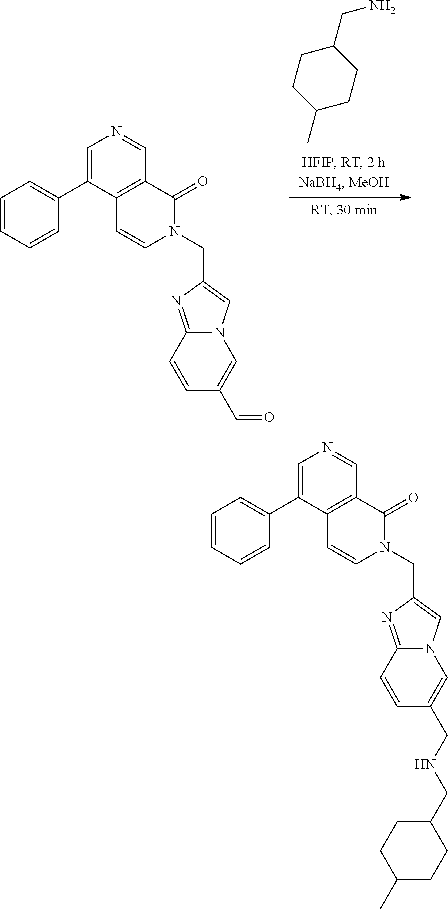

The title compound was prepared using the procedure described for example 72, step 2 with the following modification: the reaction was performed with 4-methylcyclohexylmethylamine to give 2-{[6-({[(4-methylcyclohexyl)methyl]amino}methyl)imidazo[1,2-a]pyridin-2-yl]methyl}-5-phenyl-1,2-dihydro-2,7-naphthyridin-1-one (yield: 26%) as an off-white solid. ¹H NMR (400 MHz, DMSO-d₆): δ 9.39 (s, 1H), 8.66 (s, 1H), 8.38 (d, J=3.2 Hz, 1H), 7.86 (s, 1H), 7.84 (s, 1H)₇.57-7.48 (m, 5H), 7.43 (dd, J=9.2, 2.0 Hz, 1H), 7.23 (ddd, J=9.2, 4.8, 1.2 Hz, 1H), 6.47 (d, J=7.6 Hz, 1H), 5.29 (s, 2H), 3.66 (d, J=7.2 Hz, 2H), 2.41 (d, J=6.8 Hz, 1H), 2.31 (d, J=6.8 Hz, 1H), 1.73 (d, J=7.2 Hz, 1H), 1.63-1.54 (m, 2H), 1.43-1.34 (m, 2H), 1.23-1.14 (m, 2H), 0.91-0.79 (m, 6H). MS: m/z: 492 [M+H]⁺

### Synthetic Example 86: 2-((6-(((4-methylbenzyl)amino)methyl)imidazo[1,2-a]pyridin-2-yl)methyl)-5-phenyl-2,7-naphthyridin-1(2H)-one. (Compound No. 86)

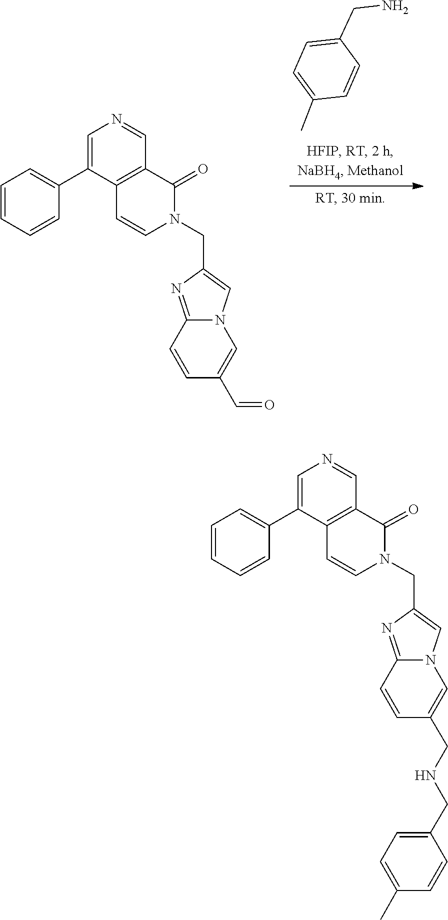

The title compound was prepared using the procedure described for example 72, step 2 with the following modification: the reaction was performed with 4-methylphenylmethylamine to give 2-{[6-({[(4-methylphenyl)methyl]amino}methyl)imidazo[1,2-a]pyridin-2-yl]methyl}-5-phenyl-1,2-dihydro-2,7-naphthyridin-1-one (yield: 33%) as an off-white solid. ¹H NMR (400 MHz, DMSO-d₆): δ 9.39 (d, J=0.8 Hz, 1H), 8.66 (s, 1H), 8.38 (s, 1H), 7.86-7.84 (m, 2H), 7.58-7.48 (m, 5H), 7.43 (d, J=9.2 Hz, 1H), 7.25-7.14 (m, 3H), 7.10 (d, J=7.6 Hz, 2H), 6.47 (dd, J=7.6, 0.4 Hz, 1H), 5.29 (s, 2H), 3.62 (s, 4H), 2.26 (s, 3H). MS: m/z: 486 [M+H]⁺

### Synthetic Example 87: N-(5-(7-((6-((((3-fluorocyclobutyl)methyl)amino)methyl)imidazo[1,2-a]pyridin-2-yl)methyl)-8-oxo-7,8-dihydro-2,7-naphthyridin-4-yl)pyridin-2-yl)acetamide. (Compound No. 87)

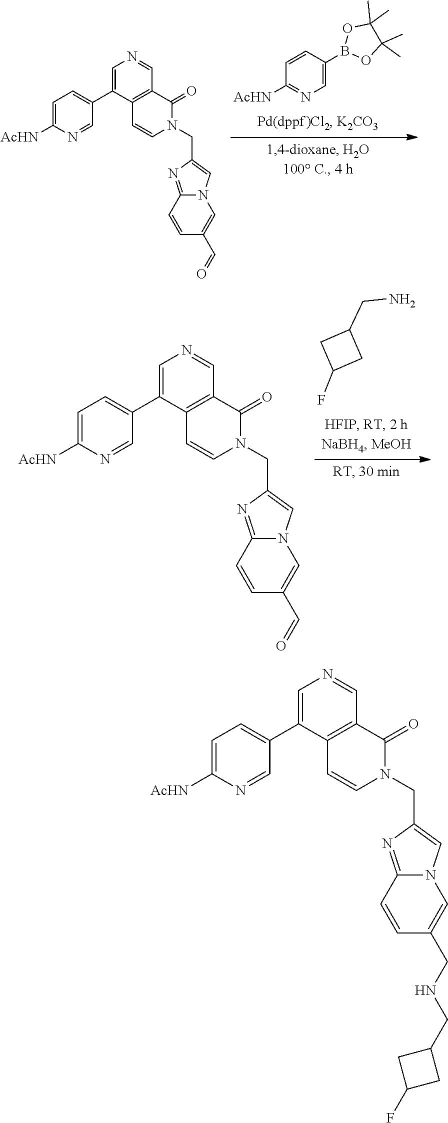 [SMILES: CC(=O)Nc1ccc(-c2cncc3c(=O)n(Cc4cn5cc(C=O)ccc5n4)ccc23)cn1 ; CC(=O)Nc1ccc(B2OC(C)(C)C(C)(C)O2)cn1 ; CC(=O)Nc1ccc(-c2cncc3c(=O)n(Cc4cn5cc(C=O)ccc5n4)ccc23)cn1 ; NCC1CC(F)C1 ; CC(=O)Nc1ccc(-c2cncc3c(=O)n(Cc4cn5cc(CNCC6CC(F)C6)ccc5n4)ccc23)cn1]

Step 1: A solution of 2-[(5-bromo-1-oxo-1,2-dihydro-2,7-naphthyridin-2-yl)methyl]imidazo[1,2-a]pyridine-6-carbaldehyde(65 mg, 170 μmol) in mixture of 1,4-dioxane & water (4:1) (10 mL) was purged with argon for 15 min, followed by the addition of N-[5-(4,4,5,5-tetramethyl-1,3,2-dioxaborolan-2-yl)pyridin-2-yl]acetamide (48.9 mg, 187 μmol), K₂CO₃ (251 mg, 1.82 mmol), palladium(II) dichloride with dichloromethane complex (27.7 mg, 33.9 μmol), and heated at 100° C. for 4 h. The reaction mixture was cooled to room temperature, diluted with ethyl acetate (20 mL), filtered through celite bed, and the filtrate was concentrated in vacuo and purified by flash chromatography (SiO₂, 70% EtOAc/Hexanes) to give N-(5-(7-((6-formylimidazo[1,2-a]pyridin-2-yl)methyl)-8-oxo-7,8-dihydro-2,7-naphthyridin-4-yl)pyridin-2-yl)acetamide (60 mg, yield: 31%) as a pale brown solid. MS: m/z: 439[M+H]⁺

Step 2: The title compound was prepared using the procedure described for example 72, step 2 with the following modification: the reaction was performed with 1-(3-fluorocyclobutyl)methanamine and N-{5-[7-({6-formylimidazo[1,2-a]pyridin-2-yl}methyl)-8-oxo-7,8-dihydro-2,7-naphthyridin-4-yl]pyridin-2-yl}acetamide and purified by reverse phase prep-HPLC (Column: YMC Triat C18 (250*21.2 mm)5μ, 0-95% CH₃CN/H₂O) to give N-[5-(7-{[6-(([(3-fluorocyclobutyl)methyl]amino}methyl)imidazo[1,2-a]pyridin-2-yl]methyl)-8-oxo-7,8-dihydro-2,7-naphthyridin-4-yl)pyridin-2-yl]acetamide (yield: 27%) as an off-white solid. ¹H NMR (400 MHz, DMSO-d₆): δ 10.73 (s, 1H), 9.40 (d, J=0.4 Hz, 1H), 8.71 (s, 1H), 8.43 (dd, J=2.4, 0.8 Hz, 1H), 8.38 (s, 1H), 8.24 (d, J=8.4 Hz, 1H), 7.94 (dd, J=8.4, 2.4 Hz, 1H), 7.88-7.87 (m, 2H), 7.44 (d, J=9.2 Hz, 1H), 7.23 (dd, J=9.2, 1.6 Hz, 1H), 6.50 (dd, J=7.6, 0.8 Hz, 1H), 5.30 (s, 2H), 5.15 (t, J=6.0 Hz, 0.5H), 5.02 (t, J=6.0 Hz, 0.5H), 3.41 (s, 2H), 2.48 (s, 2H), 2.34-2.32((m, 1H), 2.18-2.17 (m, 1H), 2.14 (s, 3H), 2.11-2.08 (m, 3H). MS: m/z: 526 [M+H]⁺

### Synthetic Example 88: 2-((6-(((cyclobutylmethyl)amino)methyl)imidazo[1,2-a]pyridin-2-yl)methyl)-5-cyclopropyl-2,7-naphthyridin-1(2 h)-one. (Compound No. 88)

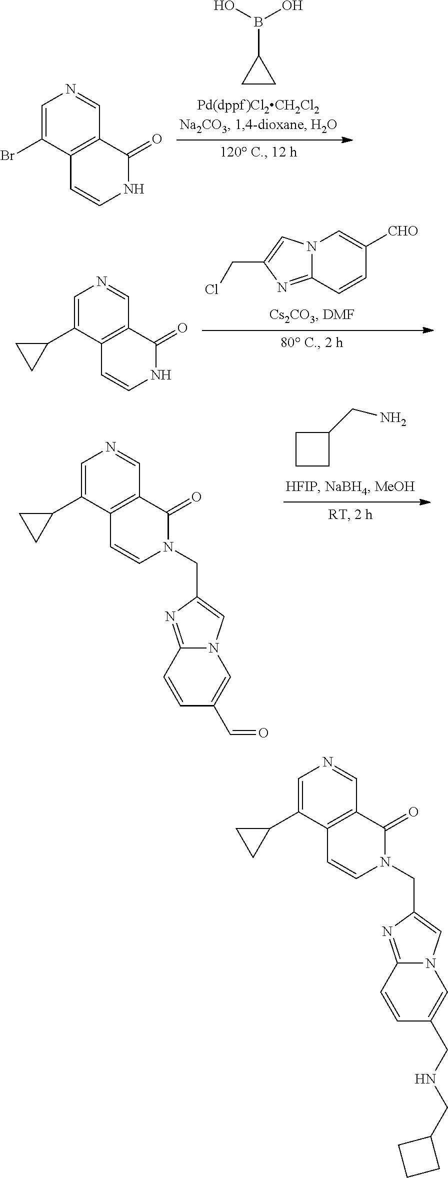

Step 1: A solution of 5-bromo-1,2-dihydro-2,7-naphthyridin-1-one(150 mg, 667 μmol) in 1,4-dioxane: water (4:1) (10 mL) was purged with argon for 10 min, followed by the addition of Na₂CO₃ (141 mg, 21.33 mmol), cyclopropylboronic acid (115 mg, 1.33 mmol) and [1,1′-Bis(diphenylphosphino)ferrocene]dichloropalladium(II) (109 mg, 133 μmol). The reaction mixture was heated at 120° C. for 12 h, cooled to room temperature, diluted with EtOAc (30 mL) and filtered through a celite pad. The filtrate was washed with water (20 mL), dried over anhydrous sodium sulphate, filtered and concentrated in vacuo and purified by mPLC (SiO₂, 2% MeOH/DCM) to afford 5-cyclopropyl-1,2-dihydro-2,7-naphthyridin-1-one (45.0 mg, yield: 36%) as a pale-yellow solid. MS: m/z: 187 [M+H]⁺

Step 2: The intermediate compound was prepared using the procedure described for example 72, step 1 with the following modification: the reaction was performed with 5-cyclopropyl-1,2-dihydro-2,7-naphthyridin-1-one to give 2-((5-cyclopropyl-1-oxo-2,7-naphthyridin-2(1H)-yl)methyl)imidazo[1,2-a]pyridine-6-carbaldehyde (yield: 90%) as a brown solid. MS: (m/z): 345 [M+H]⁺

Step 3: The title compound was prepared using the procedure described for example 72, step 2 with the following modification: the reaction was performed with 2-((5-cyclopropyl-1-oxo-2,7-naphthyridin-2(1H)-yl)methyl)imidazo[1,2-a]pyridine-6-carbaldehyde and 1-cyclobutylmethanamine and purified by reverse phase prep HPLC method (Column: YMC TRIATC18 (250*20 mm)5μ, 0-95% CH₃CN/H₂O) to afford 2-[(6-{[(cyclobutylmethyl)amino]methyl}imidazo[1,2-a]pyridin-2-yl)methyl]-5-cyclopropyl-1,2-dihydro-2,7-naphthyridin-1-one(yield: 11%) as an off-white solid. ¹H NMR (400 MHz, DMSO-d₆): δ 9.21 (s, 1H), 8.44 (d, J=0.8 Hz, 1H), 8.38 (s, 1H), 8.25 (s, 1H), 7.92 (d, J=7.6 Hz, 1H), 7.84 (s, 1H), 7.44 (d, J=9.2 Hz, 1H), 7.23 (dd, J=9.2, 1.6 Hz, 1H), 6.96 (d, J=7.6 Hz, 1H), 5.29 (s, 2H), 3.66 (s, 2H), 2.49-2.39 (m, 3H), 2.16-2.13 (m, 1H), 1.98-1.81 (m, 2H), 1.79-1.74 (m, 2H), 1.63-1.58 (m, 2H), 1.06-1.10 (m, 2H), 0.79-0.75 (m, 2H). MS: m/z: 414 [M+H]⁺

### Synthetic Example 89: Synthesis of 5-(azetidin-1-yl)-2-((6-((((3-fluorocyclobutyl)methyl)-amino)methyl)imidazo[1,2-a]pyridin-2-yl)methyl)-2,7-naphthyridin-1(2H)-one. (Compound No. 89)

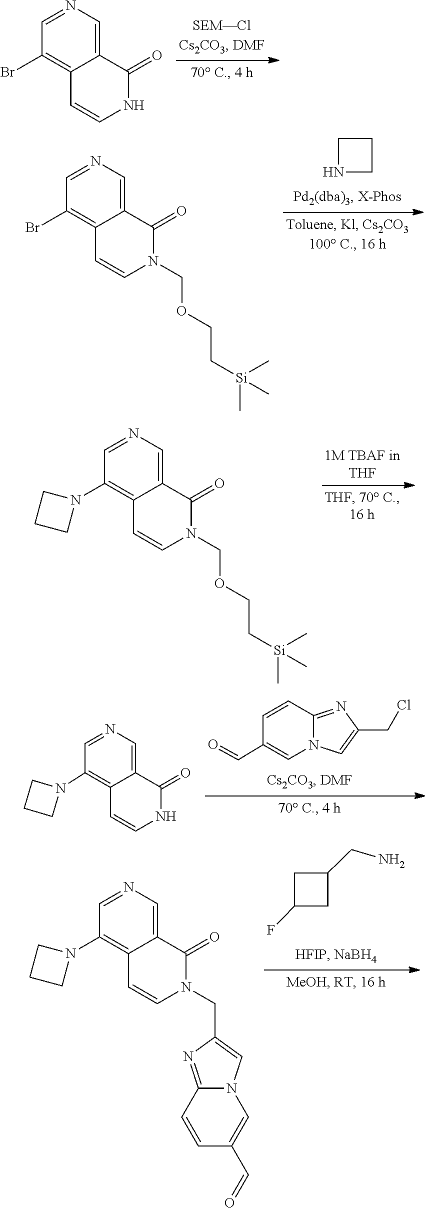

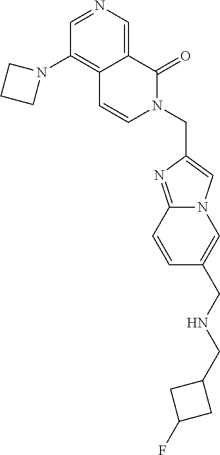 [SMILES: O=c1c2cncc(N3CCC3)c2ccn1Cc1cn2cc(CNCC3CC(F)C3)ccc2n1]

Step 1: Caesium carbonate (1.09 g, 3.33 mmol) was added to a stirred solution of 5-bromo-1,2-dihydro-2,7-naphthyridin-1-one (250 mg, 1.11 mmol) in DMF (4 mL), followed by the addition of [2-(chloromethoxy)ethyl]trimethylsilane (278 mg, 1.67 mmol). The reaction mixture was heated at 70° C. for 1 h, cooled to room temperature, diluted with ice-cold water (10 mL) and extracted with ethyl acetate (60 mL×2). The combined organic layers were dried over sodium sulphate, filtered, concentrated in vacuo and purified by flash chromatography (SiO₂, 0-100% EtOAc/hexane) to yield 5-bromo-2-{[2-(trimethylsilyl) ethoxy]methyl}-1,2-dihydro-2,7-naphthyridin-1-one (210 mg, yield: 54%) as a gummy solid. MS: m/z: 354 [M+H]⁺

Step 2: A solution of 5-bromo-2-{[2-(trimethylsilyl)ethoxy]methyl}-1,2-dihydro-2,7-naphthyridin-1-one (180 mg, 507 μmol) in DMF (6 mL) was purged with argon for 10 min, followed by the addition of azetidine(34.7 mg, 608 μmol), Cs₂CO₃ (495 mg, 1.52 mmol), dicyclohexyl[2′,4′,6′-tris(propan-2-yl)-[1,1′-biphenyl]-2-yl]phosphane (48.3 mg, 101 μmol) and tris(1,5-diphenylpenta-1,4-dien-3-one) dipalladium (92.8 mg, 101 mol) and heated at 100° C. for 16 h. After cooling to room temperature the reaction mixture was filtered through a celite pad, and the filtrate was diluted with water (20 mL) and extracted with EtOAc (60 mL×2). The combined organic layers were washed with brine (10 mL), dried over sodium sulphate, filtered, concentrated in vacuo and purified by mPLC (SiO₂, 200-400 Mesh, 20% EtOAc/Hexane) to give 5-(azetidin-1-yl)-2-{[2-(trimethylsilyl) ethoxy]methyl}-1,2-dihydro-2,7-naphthyridin-1-one (141 mg, yield: 84) as a gummy white solid. MS: (m/z): 332 [M+H]⁺

Step 3: 1M TBAF in THF (5 mL) was added to a solution of 5-(azetidin-1-yl)-2-{[2-(trimethylsilyl) ethoxy]methyl}-1,2-dihydro-2,7-naphthyridin-1-one (140 mg, 422 μmol) in THF (10 mL), and heated at 70° C. for 16 h. The reaction mixture was cooled to room temperature, quenched with water (20 mL), and extracted with ethyl acetate (40 mL×2). The combined organic layers were dried over anhydrous sodium sulphate, filtered, concentrated in vacuo and the residue was purified by flash chromatography (SiO₂, 0-100% EtOAc/hexane) to give 5-(azetidin-1-yl)-1,2-dihydro-2,7-naphthyridin-1-one (82 mg, 407 μmol, yield: 94%) as a gummy solid. MS: m/z: 202 [M+H]⁺

Step 4: The intermediate compound was prepared using the procedure described for example 72, step 1 with the following modification: the reaction was performed with 5-(azetidin-1-yl)-1,2-dihydro-2,7-naphthyridin-1-one (80 mg, 398 μmol) to give 2-{[5-(azetidin-1-yl)-1-oxo-1,2-dihydro-2,7-naphthyridin-2-yl]methyl}imidazo[1,2-a]pyridine-6-carbaldehyde (61 mg, yield: 46%) as a gummy liquid. MS: m/z: 360 [M+H]⁺

Step 5: The title compound was prepared using the procedure described for example 72, step 2 with the following modification: the reaction was performed with 3-fluorocyclobutan-1-amine and 2-{[5-(azetidin-1-yl)-1-oxo-1,2-dihydro-2,7-naphthyridin-2-yl]methyl}imidazo[1,2-a]pyridine-6-carbaldehyde and purified by reverse phase prep HPLC method (Column: GEMINI C18 2.0*50 mm, 3 μm: 0-95% ACN/H₂O) to give 5-(azetidin-1-yl)-2-{[6-({[(3-fluorocyclobutyl)methyl]amino}methyl)imidazo[1,2-a]pyridin-2-yl]methyl}-1,2-dihydro-2,7-naphthyridin-1-one (yield: 15%) as a pale yellow solid. ¹H NMR (400 MHz, DMSO-d₆): δ 8.79 (s, 1H), 8.37 (s, 1H), 7.87 (s, 1H), 7.83 (s, 1H), 7.69 (d, J=7.6 Hz, 1H), 7.44 (d, J=9.2 Hz, 1H), 7.23 (dd, J=9.2, 1.6 Hz, 1H), 6.59 (d, J=7.6 Hz, 1H), 6.33 (bs, 1H), 5.25 (s, 2H), 5.16 (t, J=6.0 Hz, 0.5H), 5.02 (t, J=6.0 Hz, 0.5H), 4.12 (t, J=7.6 Hz, 4H), 3.64 (s, 2H), 2.46 (s, 1H), 2.37-2.33 (m, 4H), 2.20-2.05 (m, 3H). MS: m/z: 447 [M+H]⁺

### Synthetic Example 90: 4-benzyl-2-((6-(((cyclobutylmethyl)amino)methyl)imidazo[1,2-a]pyridin-2-yl)methyl)-2,7-naphthyridin-1(2H)-one. (Compound No. 90)

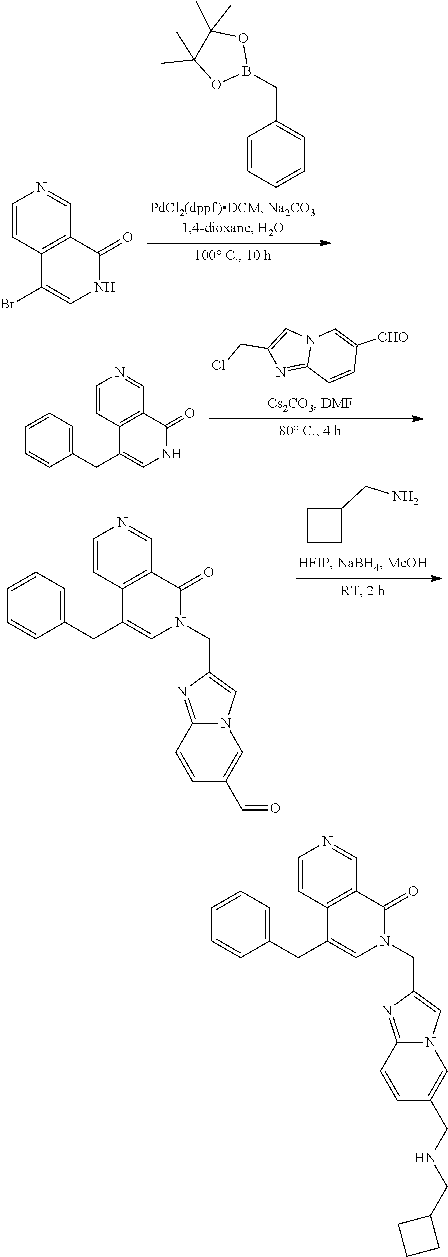

Step 1: The intermediate compound was prepared using the procedure described for example 88, step 1 with the following modification: the reaction was performed with 4-bromo-1,2-dihydro-2,7-naphthyridin-1-one and benzyl boronic acid pinacol ester to give 4-benzyl-1,2-dihydro-2,7-naphthyridin-1-one (yield: 52%) as a pale-yellow solid. MS: m/z: 237 [M+H]⁺

Step 2: The intermediate compound was prepared using the procedure described for example 72, step 1 with the following modification: the reaction was performed with 4-benzyl-1,2-dihydro-2,7-naphthyridin-1-one (50 mg, 212 μmol) to give 2-((4-benzyl-1-oxo-2,7-naphthyridin-2(1H)-yl)methyl)imidazo[1,2-]pyridine-6-carbaldehyde (50 mg, yield: 60%) as a brown solid. MS: m/z: 395 [M+H]⁺

Step 3: The title compound was prepared using the procedure described for example 72, step 2 with the following modification: the reaction was performed with 2-[(4-benzyl-1-oxo-1,2-dihydro-2,7-naphthyridin-2-yl)methyl]imidazo[1,2-a]pyridine-6-carbaldehyde (50.0 mg, 53.2 μmol) to give 4-benzyl-2-[(6-{[(cyclobutylmethyl)amino]methyl}imidazo[1,2-a]pyridin-2-yl)methyl]-1,2-dihydro-2,7-naphthyridin-1-one (5.78 mg, yield: 22%) as an off-white solid. ¹H NMR (400 MHz, DMSO-d₆): δ 9.36 (d, J=0.4 Hz, 1H), 8.67 (d, J=5.6 Hz, 1H), 8.57 (s, 1H), 7.99 (s, 1H), 7.87 (s, 1H), 7.59 (d, J=9.2 Hz, 1H), 7.50 (d, J=5.6 Hz, 1H), 7.34-7.17 (m, 6H), 5.32 (s, 2H), 4.09 (s, 2H), 4.04 (s, 2H), 2.94 (d, J=7.6 Hz, 2H), 2.56 (s, 2H), 2.09-1.99 (m, 2H), 1.91-1.69 (m, 4H). MS: m/z: 464 [M+H]⁺

### Synthetic Example 91: 2-((6-(((6-methylpyridin-2-yl)amino)methyl)imidazo[1,2-a]pyridin-2-yl)methyl)-5-phenyl-2,7-naphthyridin-1(2H)-one. (Compound No. 91)

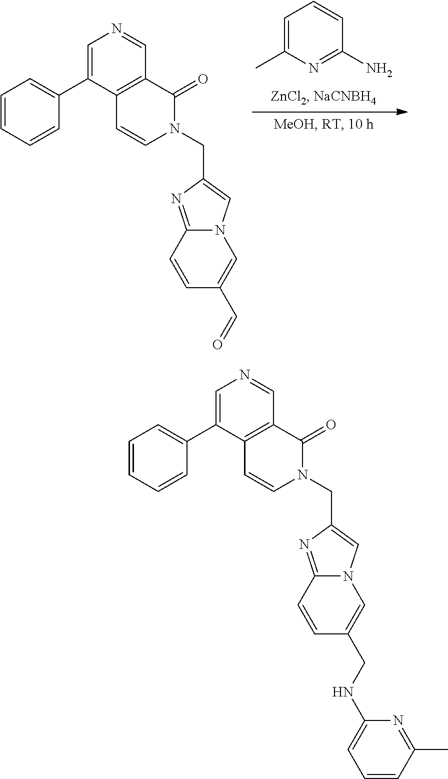

[0638]6-methylpyridin-2-amine (20.5 mg, 189 μmol) was added to a stirred solution of 2-[(1-oxo-5-phenyl-1,2-dihydro-2,7-naphthyridin-2-yl)methyl]imidazo[1,2-a]pyridine-6-carbaldehyde (60 mg, 158 μmol) in methanol (3 mL), followed by the addition of a catalytic amount of ZnCl₂ (6 mg) at room temperature. After 16 h the reaction mixture was cooled to 0° C., NaBH₄ (11.9 mg, 315 μmol) was added, followed by the addition of a few drops of methanol (0.5 mL). After 30 min, the reaction mixture was diluted with water (10 mL) and extracted with 10% methanol/DCM (25 mL×2). The combined organic layers were dried over anhydrous sodium sulphate, filtered, concentrated in vacuo and purified by reverse phase Prep-HPLC method (Column: YMC Triat C18 (250*21.2 mm)5μ, 0-95% CH3CN/H2O) to afford 2-[(6-{[(6-methylpyridin-2-yl)amino]methyl}imidazo[1,2-a]pyridin-2-yl)methyl]-5-phenyl-1,2-dihydro-2,7-naphthyridin-1-one (1.6 mg, yield: 2%) as a pale brown solid. ¹H NMR (400 MHz, DMSO-d₆): δ 9.38 (d, J=0.8 Hz, 1H), 8.66 (s, 1H), 8.40 (s, 1H), 7.88 (s, 1H), 7.84 (d, J=7.6 Hz, 1H), 7.57-7.48 (m, 5H), 7.44 (d, J=9.2 Hz, 1H), 7.38-7.20 (m, 2H), 6.91 (t, J=6.0 Hz, 1H), 6.46 (d, J=7.2 Hz, 1H), 6.35 (d, J=7.2 Hz, 1H), 6.26 (d, J=8.4 Hz, 1H), 5.28 (s, 2H), 4.40 (d, J=5.6 Hz, 2H), 2.24 (s, 3H). MS: m/z: 473 [M+H]⁺

### Synthetic Example 92: Synthesis of 2-(4-(2-(azetidin-1-yl)quinolin-7-yl)benzyl)-5-phenyl-2,7-naphthyridin-1(2H)-one. (Compound No. 92)

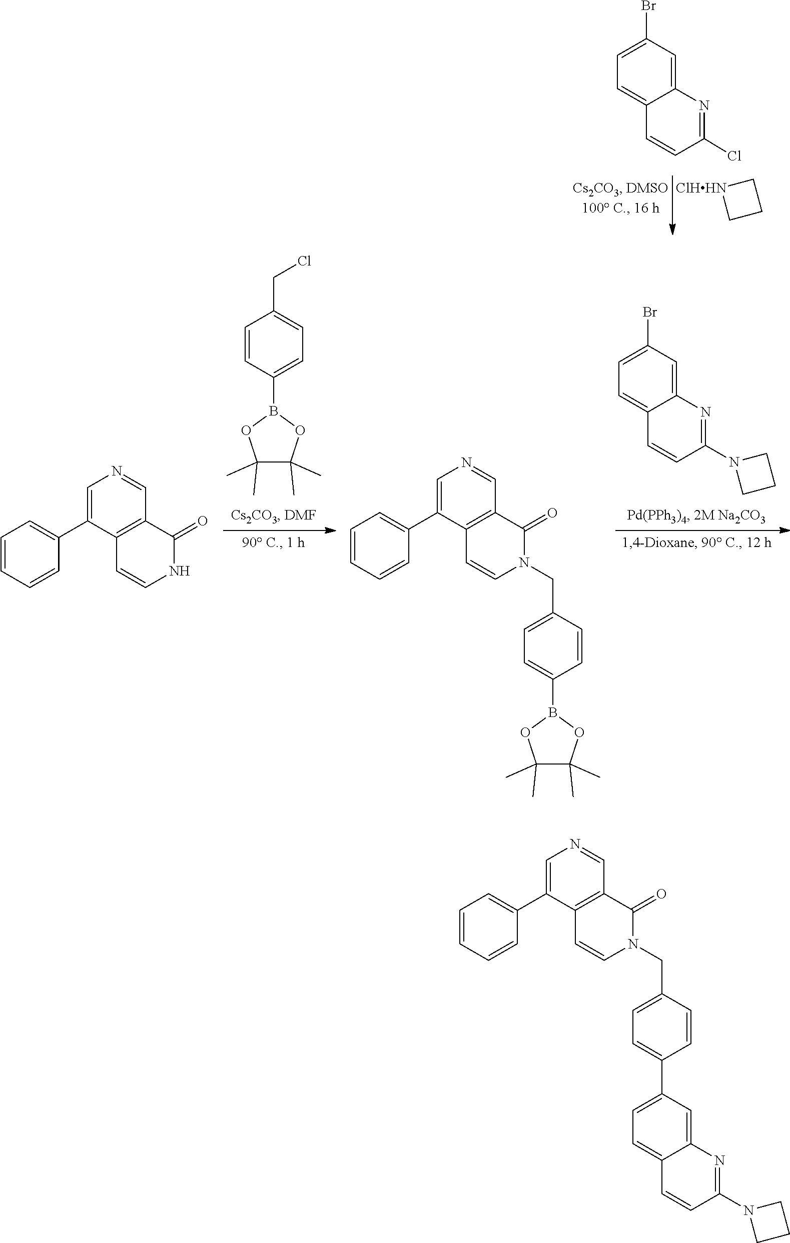

Step 1: Caesium carbonate (293 mg, 0.9 mmol) was added to a solution 5-phenyl-1,2-dihydro-2,7-naphthyridin-1-one (0.1 g 450 μmol) in DMF (2 mL) followed by the addition of 2-[4-(chloromethyl)phenyl]-4,4,5,5-tetramethyl-1,3,2-dioxaborolane (136 mg, 540 μmol). The reaction mixture was heated at 90° C. for 1 h, diluted with ice-water (15 mL) and extracted with EtOAc (25 mL×2). The combined organic layer was dried over anhydrous sodium sulphate, filtered, concentrated in vacuo and the resultant residue was purified by column chromatography (SiO₂, 60% EtOAc/hexane) to result in 5-phenyl-2-{[4-(4,4,5,5-tetramethyl-1,3,2-dioxaborolan-2-yl) phenyl]methyl}-1,2-dihydro-2,7-naphthyridin-1-one (40 mg, yield: 20%) as a pale yellow semi-solid. MS: m/z: 439 [M+H]⁺

Step 2: Caesium carbonate (304 mg, 937 mol) was added to a stirred solution of 7-bromo-2-chloroquinoline (150 mg, 625 mol) in DMSO (4 mL) followed by addition of azetidine hydrochloride (70 mg, 756 mol) at room temperature. The reaction mixture was heated at 100° C. for 16 h. The reaction mixture was cooled to room temperature, diluted with ice-cold water (10 mL), the resultant solid was filtered, washed with diethylether (10 mL) to afford 2-(azetidin-1-yl)-7-bromoquinoline (132 mg, yield: 80%) as an off-white solid. MS: m/z: 264 [M+2]⁺

Step 3: To a mixture of 5-phenyl-2-{[4-(4,4,5,5-tetramethyl-1,3,2-dioxaborolan-2-yl)phenyl]methyl}-1,2-dihydro-2,7-naphthyridin-1-one(40 mg, 91.3 μmol), and 2-(azetidin-1-yl)-7-bromoquinoline (24 mg, 91.3 μmol) in 1,4-dioxane (2 mL) was added 2M Na₂CO₃ (0.5 mL) and the reaction mixture was purged with argon for 10 min. Pd(PPh₃)₄ (21.1 mg, 18.3 μmol) was added to it and the mixture was heated at 90° C. for 12 h, cooled to room temperature, and filtered through a pad of celite. The filtrate was concentrated in vacuo and purified by reverse phase prep-HPLC (Column: Gemini (21.2*250 mm); 5 Mm, 0-95% ACN/H₂O) to give 2-({4-[2-(azetidin-1-yl)quinolin-7-yl]phenyl}methyl)-5-phenyl-1,2-dihydro-2,7-naphthyridin-1-one (9 mg, yield: 20%) as an off-white solid. ¹H NMR (400 MHz, DMSO-d₆): δ 9.43 (d, J=0.8 Hz, 1H), 8.68 (s, 1H), 8.01 (dd, J=8.8, 0.8 Hz, 1H), 7.91 (d, J=7.6 Hz, 1H), 7.78-7.74 (m, 4H), 7.58-7.45 (m, 8H), 6.69 (d, J=8.8 Hz, 1H), 6.50 (dd, J=7.6, 0.8 Hz, 1H), 5.28 (s, 2H), 4.10 (t, J=7.2 Hz, 4H), 2.36 (quin, J=7.2 Hz, 2H). MS: m/z: 495 [M+H]⁺

### Synthetic Example 93: Synthesis of 2-(4-(2-(ethylamino)quinolin-7-yl)benzyl)-5-phenyl-2,7-naphthyridin-1(2H)-one. (Compound No. 93)

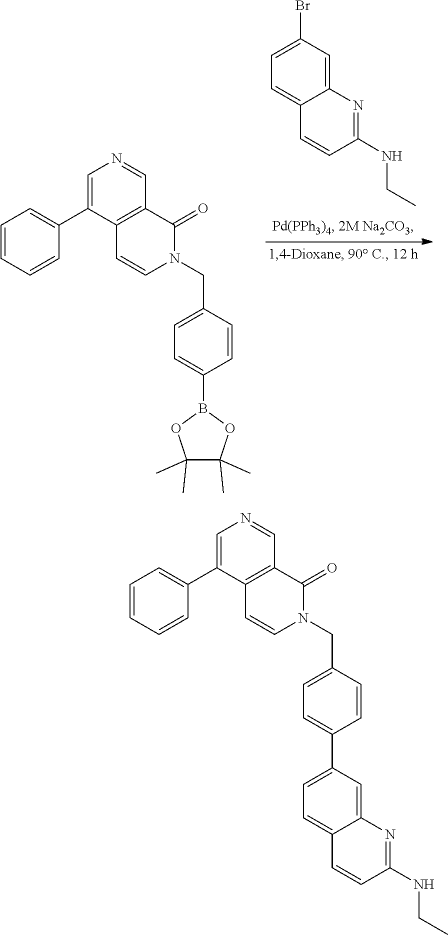

The title compound was prepared using the procedure described for example 92, step 3 with the following modification: the reaction was performed with 2-(aminoethyl)-7-bromoquinoline to give 2-({4-[2-(ethylamino)quinolin-7-yl]phenyl}methyl)-5-phenyl-1,2-dihydro-2,7-naphthyridin-1-one(yield: 15%) as a pale-yellow solid. ¹H NMR (400 MHz, DMSO-d₆): δ 9.43 (d, J=0.4 Hz, 1H), 8.68 (s, 1H), 7.91 (d, J=7.6 Hz, 1H), 7.84 (d, J=8.8 Hz, 1H), 7.76 (d, J=8.4 Hz, 1H), 7.71-7.66 (m, 3H), 7.58-7.41 (m, 8H), 7.02(bs, 1H), 6.74 (d, J=8.8 Hz, 1H), 6.50 (dd, J=7.6, 0.8 Hz, 1H), 5.28 (s, 2H), 3.43 (q, J=7.2 Hz, 2H), 1.21 (t, J=7.2 Hz, 3H); MS: m/z: 483 [M+H]⁺

### Synthetic Example 94: Synthesis of 2-(4-(2-(methylamino)quinolin-7-yl)benzyl)-5-phenyl-2,7-naphthyridin-1(2H)-one. (Compound No. 94)

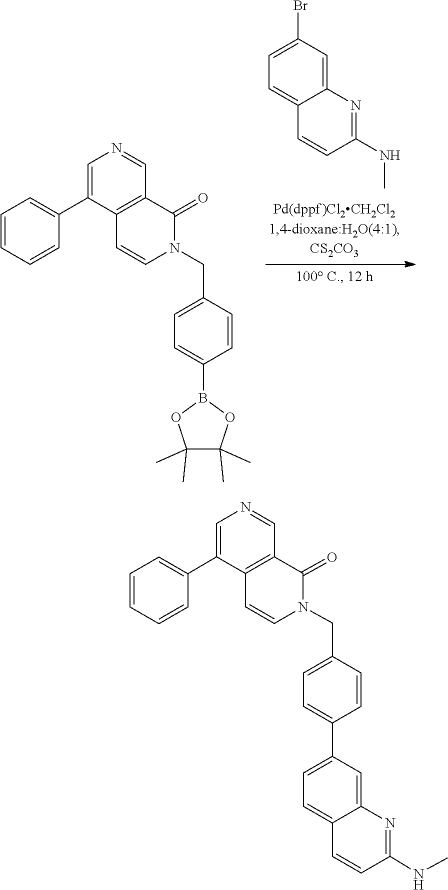

The title compound was prepared using the procedure described for example 88, step 1 with the following modification: the reaction was performed with 2-(aminomethyl)-7-bromoquinoline and 5-phenyl-2-{[4-(4,4,5,5-tetramethyl-1,3,2-dioxaborolan-2-yl) phenyl]methyl}-1,2-dihydro-2,7-naphthyridin-1-one to give 2-[(6-{[(4-methoxypyridin-2-yl) amino]methyl}Imidazo[1,2-a]pyridin-2-yl)methyl]-5-phenyl-1,2-dihydro-2,7-naphthyridin-1-one (yield: 6%) as a pale brown solid. ¹H NMR (400 MHz, DMSO-d₆): δ 9.10 (d, J=0.7 Hz, 1H), 8.59 (s, 1H), 8.28 (t, J=1.2 Hz, 1H), 7.90 (d, J=7.6 Hz, 1H), 7.78 (s, 1H), 7.40 (d, J=9.2 Hz, 1H), 7.08 (dd, J=9.2, 1.6 Hz, 1H), 6.71 (dd, J=7.6, 0.8 Hz, 1H), 5.45 (s, 2H), 5.28 (s, 2H), 2.24 (d, J=1.1 Hz, 3H). MS: m/z: 469 [M+H]⁺

### Synthetic Example 95: 2-((6-(((4-(azetidin-1-yl)pyridin-2-yl) amino)methyl)imidazo[1,2-a]pyridin-2-yl)methyl)-5-phenyl-2,7-naphthyridin-1(2H)-one. (Compound No. 95)

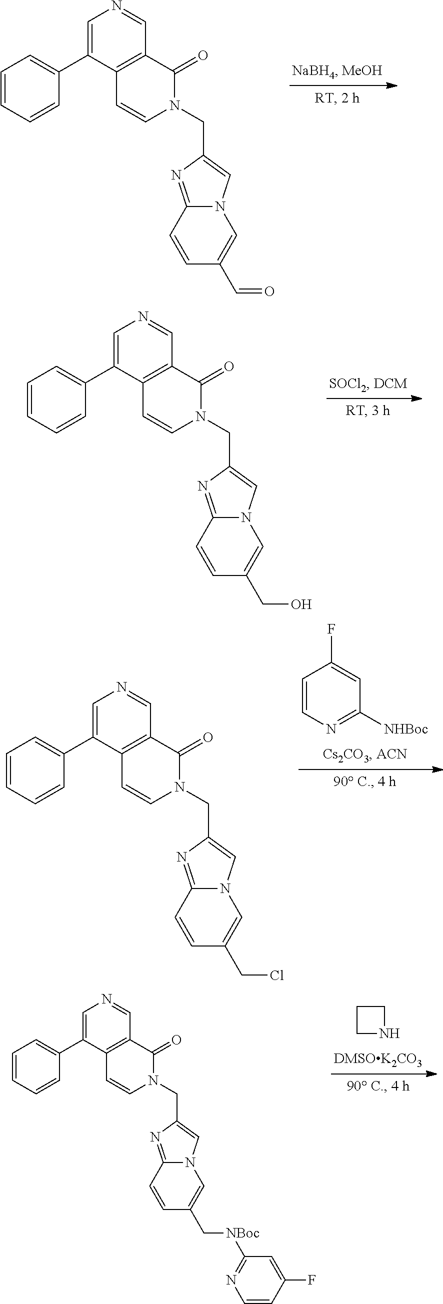

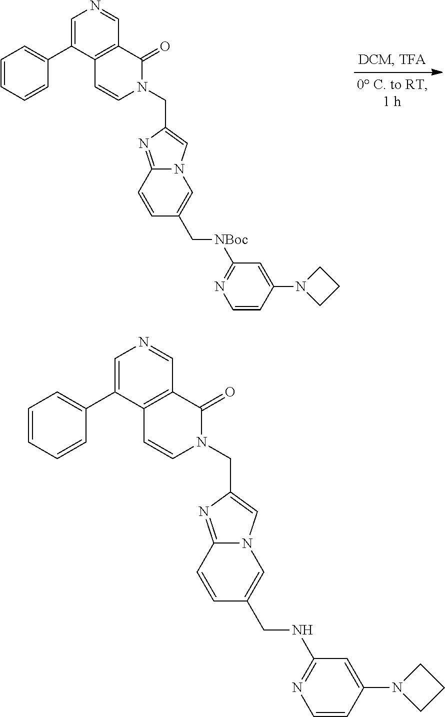 [SMILES: CC(C)(C)OC(=O)N(Cc1ccc2nc(Cn3ccc4c(-c5ccccc5)cncc4c3=O)cn2c1)c1cc(N2CCC2)ccn1 ; O=c1c2cncc(-c3ccccc3)c2ccn1Cc1cn2cc(CNc3cc(N4CCC4)ccn3)ccc2n1]

Step 1: Sodium borohydride (58.1 mg, 1.58 mmol) was added portion wise to a stirred solution of 2-[(1-oxo-5-phenyl-1,2-dihydro-2,7-naphthyridin-2-yl)methyl]imidazo[1,2-a]pyridine-6-carbaldehyde (0.3 g 789 μmol) in methanol (10 mL, 247 mmol) at 0° C. The reaction mixture was gradually warmed to room temperature and after 1 h, the reaction mixture was concentrated in vacuo, diluted with water (20 mL), and extracted with DCM (50 mL×2). The combined organic layers were dried over anhydrous sodium sulphate, filtered and concentrated in vacuo to give 2-{[6-(hydroxymethyl)imidazo[1,2-a]pyridin-2-yl]methyl}-5-phenyl-1,2-dihydro-2,7-naphthyridin-1-one (310 mg, yield: 34%) as a pale brown solid. MS: m/z: 383 [M+H]⁺

Step 2: The title compound was prepared using the procedure described for example 62, step 3 with the following modification: the reaction was performed with 2-{[6-(hydroxymethyl)imidazo[1,2-a]pyridin-2-yl]methyl}-5-phenyl-1,2-dihydro-2,7-naphthyridin-1-one (0.3 g, 784 μmol) to afford 2-{[6-(chloromethyl)imidazo[1,2-a]pyridin-2-yl]methyl}-5-phenyl-1,2-dihydro-2,7-naphthyridin-1-one (250 mg, yield: 80%) as an off-white solid. MS: m/z: 401 [M+H]⁺

Step 3: Caesium carbonate (3 equiv) was added to a stirred solution of tert-butyl N-(4-fluoropyridin-2-yl) carbamate (1.1 equiv) in acetonitrile (10 mL) followed by the addition of 2-{[6-(chloromethyl)imidazo[1,2-a]pyridin-2-yl]methyl}-5-phenyl-1,2-dihydro-2,7-naphthyridin-1-one (0.25 g). The reaction mixture was heated at 90° C. for 4 h, cooled to room temperature, diluted with water (10 mL), and extracted with 10% MeOH in DCM (20 mL×2). The combined organic layers were dried over sodium sulphate, filtered and concentrated in vacuo to afford tert-butyl N-(4-fluoropyridin-2-yl)-N-({2-[(1-oxo-5-phenyl-1,2-dihydro-2,7-naphthyridin-2-yl)methyl]imidazo[1,2-a]pyridin-6-yl}methyl) carbamate (0.2 g, yield: 56%) as a pale-brown solid. MS: m/z: 577 [M+H]⁺

Step 4: Potassium carbonate (124 mg, 173 μmol) was added to a stirred solution of tert-butylN-(4-fluoropyridin-2-yl)-N-({2-[(1-oxo-5-phenyl-1,2-dihydro-2,7-naphthyridin-2-yl)methyl]imidazo[1,2-a]pyridin-6-yl}methyl) carbamate(80 mg, 86.7 μmol) in dimethyl sulfoxide (5 mL), followed by the addition of azetidine hydrochloride (100 mg, 104 μmol). The reaction mixture was heated at 90° C. for 4 h, cooled to room temperature, diluted with ice-cold water (10 mL), and extracted with DCM (30 mL×2). The combined organic layers were washed with brine solution (10 mL), dried over anhydrous sodium sulphate, filtered and concentrated in vacuo to give of tert-butyl N-[4-(azetidin-1-yl)pyridin-2-yl]-N-({2-[(1-oxo-5-phenyl-1,2-dihydro-2,7-naphthyridin-2-yl)methyl]imidazo[1,2-a]pyridin-6-yl}methyl)carbamate (70 mg, yield: 66%) as a pale brown solid MS: m/z: 614 [M+H]⁺

Step 5: Trifluoroacetic acid (3 mL) was added to a stirred solution of tert-butyl N-[4-(azetidin-1-yl)pyridin-2-yl]-N-({2-[(1-oxo-5-phenyl-1,2-dihydro-2,7-naphthyridin-2-yl)methyl]imidazo[1,2-a]pyridin-6-yl}methyl)carbamate (50 mg, 81.5 μmol) in dichloromethane (10 mL) at room temperature. After 1 h, the reaction mixture was concentrated in vacuo and purified by reverse phase prep-HPLC (Column: YMC Triat C18 (250*21.2 mm)5μ, 0-95% CH₃CN/H₂O) to afford 2-{[6-({[4-(azetidin-1-yl)pyridin-2-yl]amino}methyl)imidazo[1,2-a]pyridin-2-yl]methyl}-5-phenyl-1,2-dihydro-2,7-naphthyridin-1-one (20 mg, yield: 47%) as a white solid. ¹H NMR (400 MHz, DMSO-d₆): δ 12.03 (s, 1H), 9.41 (s, 1H), 8.70 (s, 1H), 8.65 (s, 1H), 8.16 (s, 1H), 8.01 (t, J=6.0 Hz, 1H), 7.88 (d, J=7.6 Hz, 1H), 7.77 (d, J=9.2 Hz, 1H), 7.63-7.46 (m, 6H), 6.52 (d, J=7.6 Hz, 1H), 6.03 (dd, J=7.2, 2.0 Hz, 1H), 5.47 (s, 1H), 5.41 (s, 2H), 4.55 (d, J=6.0 Hz, 2H), 4.06 (s, 4H), 2.38-2.33 (m, 2H). MS: m/z: 514 [M+H]⁺

### Synthetic Example 96: 2-((6-(((4-((3-chloropropyl)amino)pyridin-2-yl)amino)methyl)imidazo [1,2-a]pyridin-2-yl)methyl)-5-phenyl-2,7-naphthyridin-1(2H)-one. (Compound No. 96)

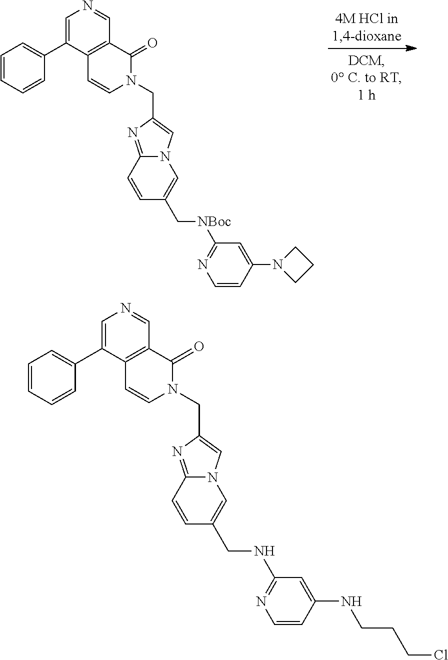 [SMILES: CC(C)(C)OC(=O)N(Cc1ccc2nc(Cn3ccc4c(-c5ccccc5)cncc4c3=O)cn2c1)c1cc(N2CCC2)ccn1 ; O=c1c2cncc(-c3ccccc3)c2ccn1Cc1cn2cc(CNc3cc(NCCCCl)ccn3)ccc2n1]

The title compound was prepared using the procedure described for example 4, step 2 with the following modification: the reaction was performed with tert-butyl (4-(azetidin-1-yl)pyridin-2-yl)(2-((1-oxo-5-phenyl-2,7-naphthyridin-2(1H)-yl)methyl)imidazo[1,2-a]pyridin-6-yl)methyl)carbamate (50 mg, 0.0815 mmol) and purified by prep-HPLC (Column: GEMINI C18(250*21.2 MM*5 u), 0-95% CH₃CN/H₂O) to afford 2-((6-(((4-((3-chloropropyl)amino)pyridin-2-yl)amino)methyl)imidazo[1,2-a]pyridin-2-yl)methyl)-5-phenyl-2,7-naphthyridin-1(2H)-one (6.2 mg, yield: 14%) as an off white solid. ¹H NMR (400 MHz, DMSO-d₆): δ 9.38 (d, J=0.8 Hz, 1H), 8.66 (s, 1H), 8.38 (s, 1H), 7.87 (s, 1H), 7.84 (d, J=7.6 Hz, 1H), 7.57-7.48 (m, 6H), 7.45 (d, J=9.2 Hz, 1H), 7.21 (dd, J=9.2, 1.6 Hz, 1H), 7.04 (s, 1H), 6.59 (s, 1H), 6.46 (dd, J=7.6, 0.8 Hz, 1H), 5.94 (dd, J=6.0, 1.6 Hz, 1H), 5.60 (d, J=2.0 Hz, 1H), 5.29 (s, 2H), 4.38 (d, J=6.0 Hz, 2H), 3.67 (t, J=6.4 Hz, 2H), 3.12 (q, J=6.4 Hz, 2H), 1.90 (p, J=6.4 Hz, 2H). MS: m/z: 550 [M+H]⁺

### Synthetic Example 97: 2-((6-(((4-(methylamino)pyridin-2-yl) amino)methyl)imidazo[1,2-a]pyridin-2-yl)methyl)-5-phenyl-2,7-naphthyridin-1(2H)-one. (Compound No. 97)

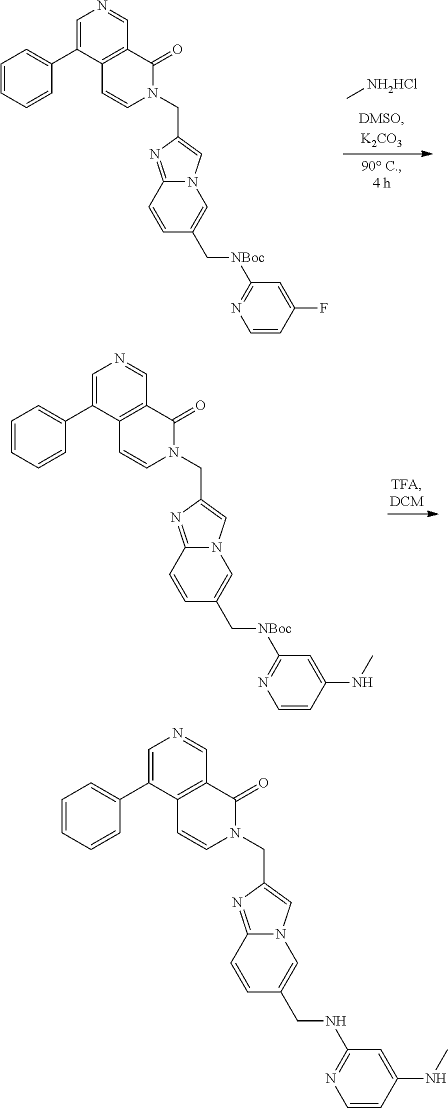

Step 1: The intermediate compound was prepared using the procedure described for example 95, step 4 with the following modification: the reaction was performed with methyl amine hydrochloride to give tert-butyl (4-(methylamino)pyridin-2-yl)(2-((1-oxo-5-phenyl-2,7-naphthyridin-2(1H)-yl)methyl)imidazo[1,2-a]pyridin-6-yl)methyl) carbamate (60 mg, yield: 73%) as a pale brown solid. MS: m/z: 588 [M+H]⁺

Step 2: The title compound was prepared using the procedure described for example 95, step 5 with the following modification: the reaction was performed with tert-butyl N-[4-(methylamino)pyridin-2-yl]-N-({2-[(1-oxo-5-phenyl-1,2-dihydro-2,7-naphthyridin-2-yl)methyl]imidazo[1,2-a]pyridin-6-yl}methyl)carbamate (45 mg, 76.6 μmol) to give 2-{[6-({[4-(methylamino)pyridin-2-yl]amino}methyl)imidazo[1,2-a]pyridin-2-yl]methyl}-5-phenyl-1,2-dihydro-2,7-naphthyridin-1-one (11 mg, yield: 29%) as a white solid. ¹H NMR (400 MHz, DMSO-d₆): δ 9.39 (s, 1H), 8.67 (s, 1H), 8.51 (s, 1H), 8.01 (s, 1H), 7.95 (s, 1H), 7.86 (d, J=7.6 Hz, 1H), 7.58-7.48 (m, 7H), 7.27 (d, J=9.2 Hz, 1H), 6.48 (d, J=7.6 Hz, 1H), 6.17 (d, J=6.0 Hz, 1H), 5.68 (s, 1H), 5.32 (s, 2H), 4.48 (s, 2H), 2.74 (d, J=4.0 Hz, 3H). MS: m/z: 488 [M+H]⁺

### Synthetic Example 98: 2-((6-(((4-(dimethylamino)pyridin-2-yl)amino)methyl)imidazo[1,2-a]pyridin-2-yl)methyl)-5-phenyl-2,7-naphthyridin-1(2H)-one. (Compound No. 98)

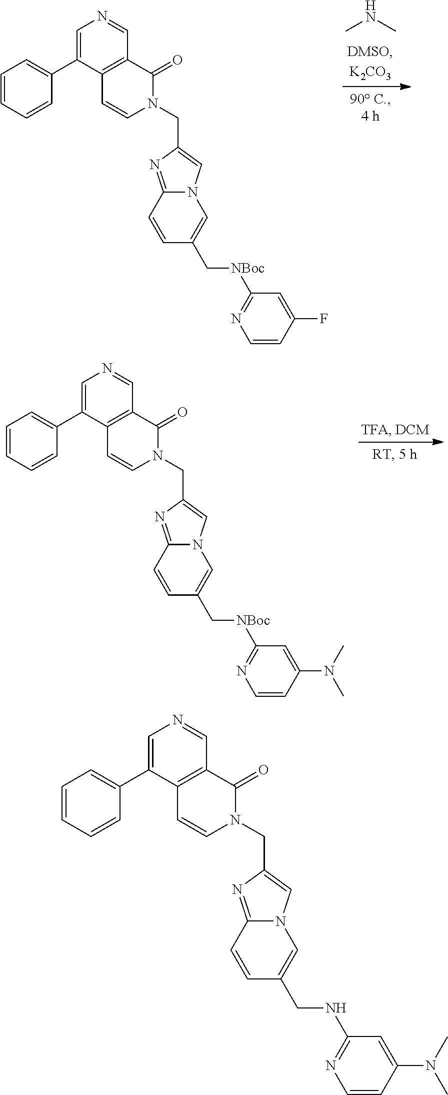

Step 1: The intermediate compound was prepared using the procedure described for example 95, step 4 with the following modification: the reaction was performed with N,N-dimethylamine to give tert-butyl (4-(dimethylamino)pyridin-2-yl)(2-((1-oxo-5-phenyl-2,7-naphthyridin-2(1H)-yl)methyl)imidazo[1,2-a]pyridin-6-yl)methyl) carbamate (50 mg, yield: 95%) as a pale brown solid. MS: m/z: 602 [M+H]⁺

Step 2: The title compound was prepared using the procedure described for example 95, step 5 with the following modification: the reaction was performed with tert-butyl N-[4-(dimethylamino)pyridin-2-yl]-N-({2-[(1-oxo-5-phenyl-1,2-dihydro-2,7-naphthyridin-2-yl)methyl]imidazo[1,2-a]pyridin-6-yl}methyl)carbamate (50 mg, 83.1 μmol) to give 2-{[6-({[4-(dimethylamino)pyridin-2-yl]amino}methyl)imidazo[1,2-a]pyridin-2-yl]methyl}-5-phenyl-1,2-dihydro-2,7-naphthyridin-1-one (4 mg, yield: 9%) as a gummy solid. ¹H NMR (400 MHz, DMSO-d₆): δ 9.40 (s, 1H), 8.68 (s, 1H), 8.58 (s, 1H), 8.04 (s, 1H), 7.93 (t, J=6.0 Hz, 1H), 7.86 (d, J=7.6 Hz, 1H), 7.63-7.62 (m, 2H), 7.56 (d, J=7.6 Hz, 2H), 7.53-7.49 (m, 3H), 7.43 (d, J=9.0 Hz, 1H), 6.50 (d, J=7.6 Hz, 1H), 6.41 (dd, J=7.6, 2.4 Hz, 1H), 5.77 (s, 1H), 5.36 (s, 2H), 4.53 (d, J=6.0 Hz, 2H), 3.04 (s, 6H). MS: m/z: 502 [M+H]⁺

### Synthetic Example 99: 2-((6-(((4-((2-methoxyethyl)amino)pyridin-2-yl)amino)methyl)imidazo[1,2-a]pyridin-2-yl)methyl)-5-phenyl-2,7-naphthyridin-1(2H)-one. (Compound No. 99)

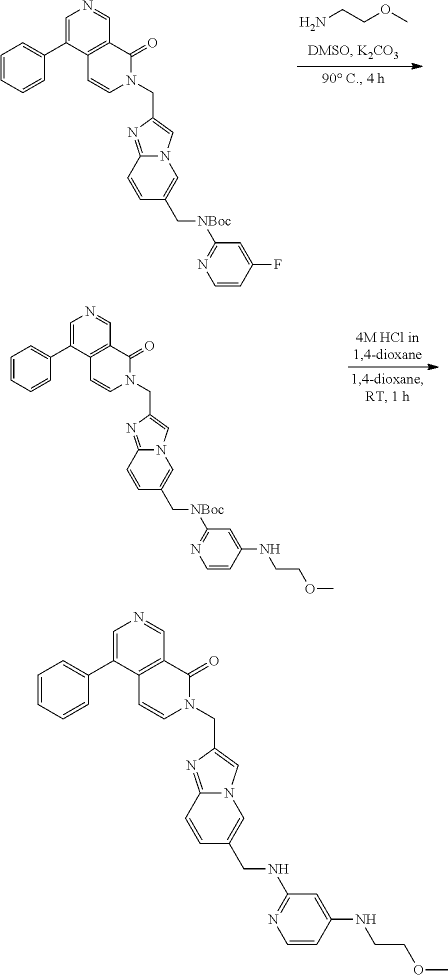

Step 1: The intermediate compound was prepared using the procedure described for example 95, step 4 with the following modification: the reaction was performed with 2-methoxyethylamine to give tert-butyl (4-((2-methoxyethyl)amino)pyridin-2-yl)(2-((1-oxo-5-phenyl-2,7-naphthyridin-2(1H)-yl)methyl)imidazo[1,2-a]pyridin-6-yl)methyl)carbamate (50 mg yield: 9%) as a pale brown solid. MS: m/z: 632 [M+H]⁺

Step 2: The title compound was prepared using the procedure described for example 95, step 5 with the following modification: the reaction was performed with tert-butyl (4-((2-methoxyethyl)amino)pyridin-2-yl)(2-((1-oxo-5-phenyl-2,7-naphthyridin-2(1H)-yl)methyl)imidazo[1,2-a]pyridin-6-yl)methyl)carbamate and purified by prep-HPLC (Column: GEMINI C18(250*21.2 MM*5 u), 0-95% CH₃CN/H₂O) to afford 2-({6-[({4-[(2-methoxyethyl) amino]pyridin-2-yl}amino)methyl]imidazo[1,2-a]pyridin-2-yl}methyl)-5-phenyl-1,2-dihydro-2,7-naphthyridin-1-one (5.2 mg, yield: 11%) as a white solid. ¹H NMR (400 MHz, DMSO-d₆): δ 9.38 (s, 1H), 8.66 (s, 1H), 8.36 (s, 1H), 7.87 (s, 1H), 7.84 (d, J=7.6 Hz, 1H), 7.57-7.48 (m, 6H), 7.43 (d, J=9.2, 1H), 7.21 (dd, J=9.2, 1.6 Hz, 1H), 6.84 (s, 1H), 6.46 (d, J=7.6 Hz, 1H), 6.30 (s, 1H), 5.91 (dd, J=6.0, 1.6 Hz, 1H), 5.59 (d, J=2.0 Hz, 1H), 5.28 (s, 2H), 4.36 (d, J=5.6 Hz, 2H), 3.39 (t, J=5.6 Hz, 2H), 3.21 (s, 3H), 3.14(q, J=5.6 Hz, 2H). MS: m/z: 532 [M+H]⁺

### Synthetic Example 100: Synthesis of 5-phenyl-2-((6-((pyridin-2-ylamino)methyl)imidazo[1,2-a]pyridin-2-yl)methyl)-2,7-naphthyridin-1(2H)-one. (Compound No. 100)

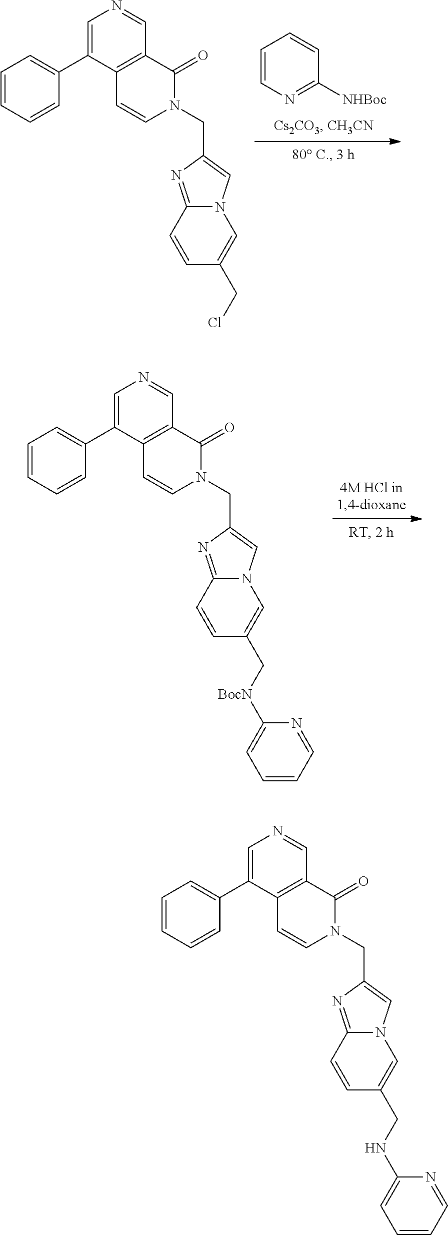 [SMILES: O=c1c2cncc(-c3ccccc3)c2ccn1Cc1cn2cc(CCl)ccc2n1 ; CC(C)(C)OC(=O)Nc1ccccn1 ; CC(C)(C)OC(=O)N(Cc1ccc2nc(Cn3ccc4c(-c5ccccc5)cncc4c3=O)cn2c1)c1ccccn1 ; O=c1c2cncc(-c3ccccc3)c2ccn1Cc1cn2cc(CNc3ccccn3)ccc2n1]

Step 1: The intermediate compound was prepared using the procedure described for example 95, step 3 with the following modification: the reaction was performed with tert-butylpyridine-2-carbamate to give tert-butyl ((2-((1-oxo-5-phenyl-2,7-naphthyridin-2(1H)-yl)methyl)imidazo[1,2-a]pyridin-6-yl)methyl)(pyridin-2-yl)carbamate (50 mg, yield: 60%) as a pale-brown solid. MS: m/z: 559 [M+H]⁺

Step 2: The intermediate compound was prepared using the procedure described for example 95, step 5 with the following modification: the reaction was performed with tert-butyl ((2-((1-oxo-5-phenyl-2,7-naphthyridin-2(1H)-yl)methyl)imidazo[1,2-a]pyridin-6-yl)methyl)(pyridin-2-yl) carbamate (50 mg, 0.089 mmol) to give 5-phenyl-2-((6-((pyridin-2-ylamino)methyl)imidazo[1,2-a]pyridin-2-yl)methyl)-2,7-naphthyridin-1(2H)-one (2.1 mg, yield: 4%) as an off-white solid. ¹H NMR (400 MHz, DMSO-d₆): δ 9.38 (s, 1H), 8.66 (s, 1H), 8.39 (s, 1H), 7.95 (dd, J=4.8, 1.6 Hz, 1H), 7.87 (s, 1H), 7.83 (d, J=7.6 Hz, 1H), 7.58-7.52 (m, 2H), 7.53-7.47 (m, 3H), 7.44 (d, J=9.2 Hz, 1H), 7.36 (dt, J=8.8, 2.0 Hz, 1H), 7.23 (dd, J=9.2, 1.6 Hz, 1H), 7.04 (t, J=6.0 Hz, 1H), 6.53-6.45 (m, 2H), 5.28 (s, 2H), 4.42 (d, J=6.0 Hz, 2H). MS: m/z: 459 [M+H]⁺

### Synthetic Example 101: 2-((6-(((4-methoxypyndin-2-yl)amino)methyl)imidazo[1,2-a]pyridin-2-yl)methyl)-5-phenyl-2,7-naphthyridin-1(2H)-one. (Compound No. 101)

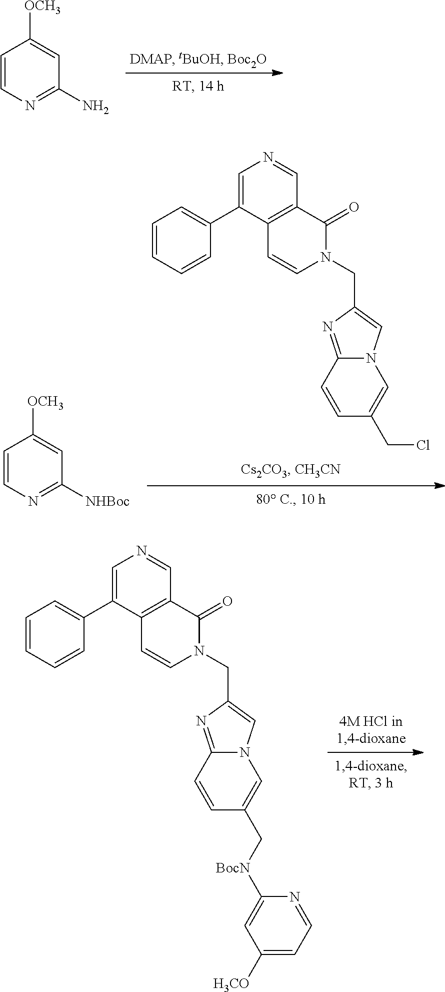 [SMILES: COc1ccnc(N)c1 ; COc1ccnc(NC(=O)OC(C)(C)C)c1 ; O=c1c2cncc(-c3ccccc3)c2ccn1Cc1cn2cc(CCl)ccc2n1 ; COc1ccnc(N(Cc2ccc3nc(Cn4ccc5c(-c6ccccc6)cncc5c4=O)cn3c2)C(=O)OC(C)(C)C)c1]

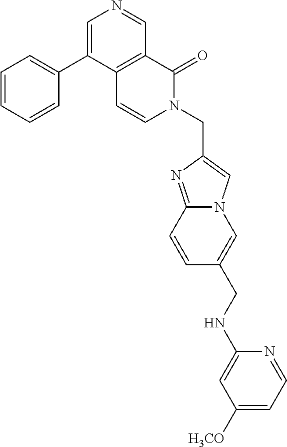 [SMILES: COc1ccnc(NCc2ccc3nc(Cn4ccc5c(-c6ccccc6)cncc5c4=O)cn3c2)c1]

Step 1: Di-tert-butyl dicarbonate (0.96 g, 0.004 mmd) was added to a stirred solution of 4-methoxypyridin-2-amine (500 mg, 0.004 mmd) in tert-butyl alcohol (10 mL), followed by the addition of a catalytic amount of DMAP (0.73 g, 0.006 mmol) at 0° C. The reaction mixture was gradually warmed to room temperature, stirred for 14 h, diluted with water (20 mL), and extracted with DCM (50 mL×2). The combined organic layer was dried over sodium sulphate, filtered and concentrated in vacuo to get tert-butyl (4-methoxypyridin-2-yl)carbamate (500 mg, yield: 55%) as a pale brown solid. MS: m/z: 225 [M+H]⁺

Step 2: The intermediate compound was prepared using the procedure described for example 95, step 4 with the following modification: the reaction was performed with tert-butyl (4-methoxypyridin-2-yl)carbamate in acetonitrile (5 mL) to give 2-[(6-{[(4-methoxypyridin-2-yl)amino]methyl}imidazo[1,2-a]pyridin-2-yl)methyl]-5-phenyl-1,2-dihydro-2,7-naphthyridin-1-one (80 mg, yield: 85%) as a pale brown solid. MS: m/z: 589 [M+H]⁺

Step 3: The title compound was prepared using the procedure described for example 95, step 5 with the following modification: the reaction was performed with 2-[(6-{[(4-methoxypyridin-2-yl)amino]methyl}imidazo[1,2-a]pyridin-2-yl)methyl]-5-phenyl-1,2-dihydro-2,7-naphthyridin-1-one (80 mg, 0.136 mmol to give 2-[(6-{[(4-methoxypyridin-2-yl)amino]methyl}imidazo[1,2-a]pyridin-2-yl)methyl]-5-phenyl-1,2-dihydro-2,7-naphthyridin-1-one (8 mg, yield: 9%) as a pale brown solid. ¹H NMR (400 MHz, DMSO-d₆): δ 9.38 (s, 1H), 8.66 (s, 1H), 8.37 (s, 1H), 7.88 (s, 1H), 7.83 (d, J=7.6 Hz, 1H), 7.77 (d, J=6.0 Hz, 1H), 7.57-7.42 (m, 5H), 7.43 (d, J=9.2 Hz, 1H), 7.22 (dd, J=9.2, 1.6 Hz, 1H), 6.96 (t, J=6.0 Hz, 1H), 6.46 (d, J=7.6 Hz, 1H), 6.15 (dd, J=6.0, 2.4 Hz, 1H), 6.00 (d, J=2.0 Hz, 1H), 5.28 (s, 2H), 4.42 (d, J=6.0 Hz, 2H), 3.69 (s, 3H). MS: m/z: 489 [M+H]⁺

### Synthetic Example 102: 2-{[6-({[4-(morpholin-4-yl)pyridin-2-yl]amino}methyl)imidazo[1,2-a]pyridin-2-yl]methyl}-5-phenyl-1,2-dihydro-2,7-naphthyridin-1-one. (Compound No. 102)

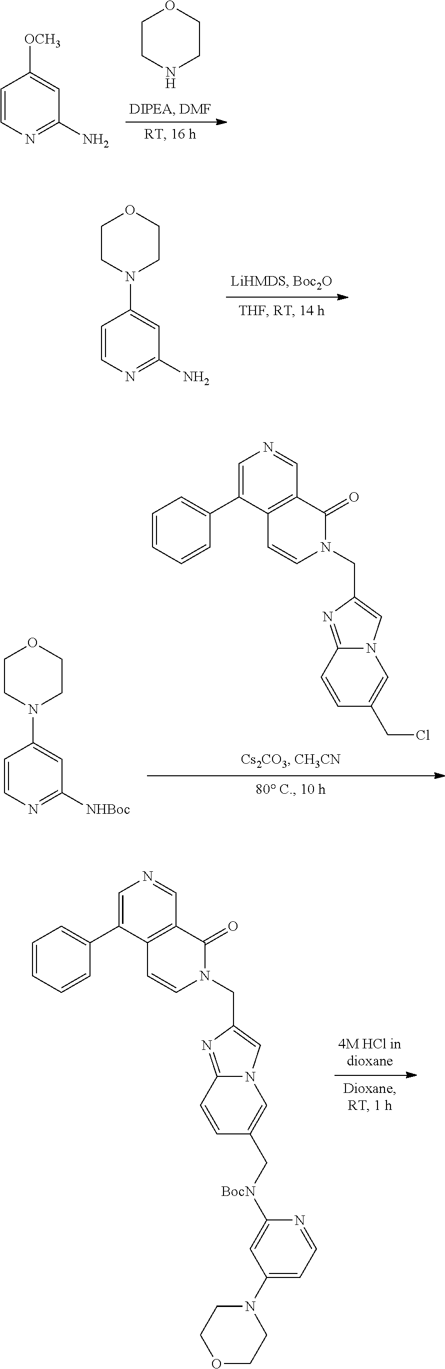 [SMILES: COc1ccnc(N)c1 ; C1COCCN1 ; Nc1cc(N2CCOCC2)ccn1 ; CC(C)(C)OC(=O)Nc1cc(N2CCOCC2)ccn1 ; O=c1c2cncc(-c3ccccc3)c2ccn1Cc1cn2cc(CCl)ccc2n1 ; CC(C)(C)OC(=O)N(Cc1ccc2nc(Cn3ccc4c(-c5ccccc5)cncc4c3=O)cn2c1)c1cc(N2CCOCC2)ccn1]

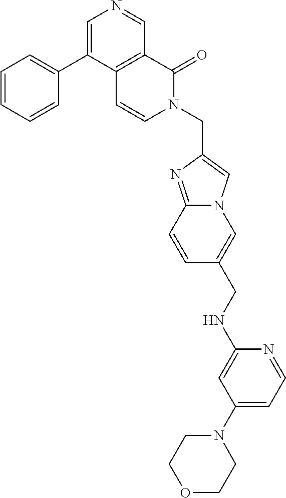 [SMILES: O=c1c2cncc(-c3ccccc3)c2ccn1Cc1cn2cc(CNc3cc(N4CCOCC4)ccn3)ccc2n1]

Step 1: DIPEA (1.2 g 0.0093 mmol) was added to a stirred solution of 4-chloropyridin-2-amine (400 mg, 0.0031 mmol) in DMF (10 mL) at 0° C., followed by the addition of morpholine (0.40 g 0.0046 mmol). After 16 h at room temperature the reaction mixture was diluted with water (20 mL) and extracted with DCM (50 mL×2). The combined organic layers were washed with brine solution (10 mL), dried over sodium sulphate, filtered and concentrated in vacuo to afford 4-morpholinopyridin-2-amine (400 mg, yield: 73%) as a pale brown solid. MS: m/z: 180 [M+H]⁺

Step 2: 1.0 M LiHMDS in THF (0.0013 mmol, 5 mL) was added dropwise to a stirred solution of 4-morpholinopyridin-2-amine (200 mg, 0.0011 mmol) in THF (10 vol) at 0° C., followed by the addition of Boc₂O (0.36 g 0.0016 mmol). After 14 h at room temperature, the reaction mixture was diluted with water (20 mL), and extracted with DCM (50 mL×2). The combined organic layers were dried over sodium sulphate, filtered and concentrated in vacuo to give tert-butyl (4-morpholinopyridin-2-yl)carbamate (250 mg, yield: 80%) as a pale brown solid. MS: m/z: 280.1 [M+H]⁺

Step 3: The intermediate compound was prepared using the procedure described for example 95, step 4 with the following modification: the reaction was performed with tert-butyl (4-morpholinopyridin-2-yl)carbamate to give tert-butyl N-[4-(morpholin-4-yl)pyridin-2-yl]-N-({2-[(1-oxo-5-phenyl-1,2-dihydro-2,7-naphthyridin-2-yl)methyl]imidazo[1,2-a]pyridin-6-yl}methyl)carbamate (yield: 38%) as a pale brown solid. MS: m/z: 630 [M+H]⁺

Step 4: The title compound was prepared using the procedure described for example 95, step 5 with the following modification: the reaction was performed with tert-butyl N-[4-(morpholin-4-yl)pyridin-2-yl]-N-({2-[(1-oxo-5-phenyl-1,2-dihydro-2,7-naphthyridin-2-yl)methyl]imidazo[1,2-a]pyridin-6-yl}methyl)carbamate (50 mg, 77.7 μmol) to give 2-{[6-({[4-(morpholin-4-yl)pyridin-2-yl]amino}methyl)imidazo[1,2-a]pyridin-2-yl]methyl}-5-phenyl-1,2-dihydro-2,7-naphthyridin-1-one (7 mg, yield: 7%) as a pale brown solid. ¹H NMR (400 MHz, DMSO-d₆): δ 9.38 (d, J=0.8 Hz, 1H), 8.66 (s, 1H), 8.36 (s, 1H), 7.88 (s, 1H), 7.84 (d, J=7.6 Hz, 1H), 7.66 (d, J=6.0 Hz, 1H), 7.57-7.53 (m, 2H), 7.52-7.48 (m, 3H), 7.43 (d, J=9.2 Hz, 1H), 7.21 (dd, J=9.2, 1.6 Hz, 1H), 6.73 (s, 1H), 6.46 (dd, J=7.6, 0.8 Hz, 1H), 6.19 (dd, J=6.0, 0.8 Hz, 1H), 5.87 (d, J=2.4 Hz, 1H), 5.28 (s, 2H), 4.40 (d, J=6.0 Hz, 2H), 3.68 (t, J=4.4 Hz, 4H), 3.12 (t, J=4.8 Hz, 4H). MS: m/z: 544 [M+H]⁺

### Synthetic Example 103: N-((2-((1-oxo-5-phenyl-2,7-naphthyridin-2(1H)-yl)methyl)imidazo[1,2-a]pyridin-6-yl)methyl)benzimidamide. (Compound No. 103)

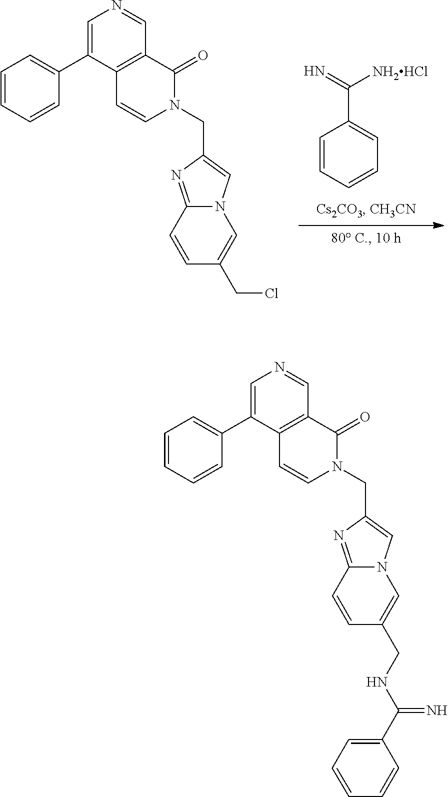

Caesium carbonate (81.3 mg, 249 μmol) was added to a stirred solution of benzene carboximidamide (15 mg, 125 μmol) in acetonitrile (10 mL), followed by the addition of 2-{[6-(chloromethyl)imidazo[1,2-a]pyridin-2-yl]methyl}-5-phenyl-1,2-dihydro-2,7-naphthyridin-1-one (50 mg, 125 μmol). The reaction mixture was heated at 80° C. for 10 h, diluted with water (10 mL), and extracted with 10% methanol in DCM (20 mL×2). The combined organic layers were dried over anhydrous sodium sulphate, filtered, concentrated in vacuo and purified by reverse phase prep-HPLC (Column: YMC Triat C18 (250*21.2 mm)5 μA, 0-95% CH₃CN/H₂O) to afford N-({2-[(1-oxo-5-phenyl-1,2-dihydro-2,7-naphthyridin-2-yl)methyl]imidazo[1,2-a]pyridin-6-yl}methyl)benzene carboximidamide (5.1 mg, yield: 8%) as a white solid. ¹H NMR (400 MHz, DMSO-d₆): δ 9.39 (d, J=0.4 Hz, 1H), 8.67 (d, J=1.6 Hz, 1H), 8.57 (s, 1H), 8.35 (s, 1H), 7.93 (s, 1H), 7.87 (d, J=7.6 Hz, 1H), 7.81 (d, J=7.2 Hz, 2H), 7.61-7.49((m, 10H), 7.29 (dd, J=9.2, 1.6 Hz, 1H), 6.48 (d, J=8.0 Hz, 1H), 5.31 (s, 2H), 4.53 (s, 2H). MS: m/z: 485 [M+H]⁺

### Synthetic Example 104: 2-((6-(6-((cyclobutylmethyl)amino)pyridin-2-yl)imidazo[1,2-a]pyridin-2-yl)methyl)-5-phenyl-2,7-naphthyridin-1(2H)-one. (Compound No. 104)

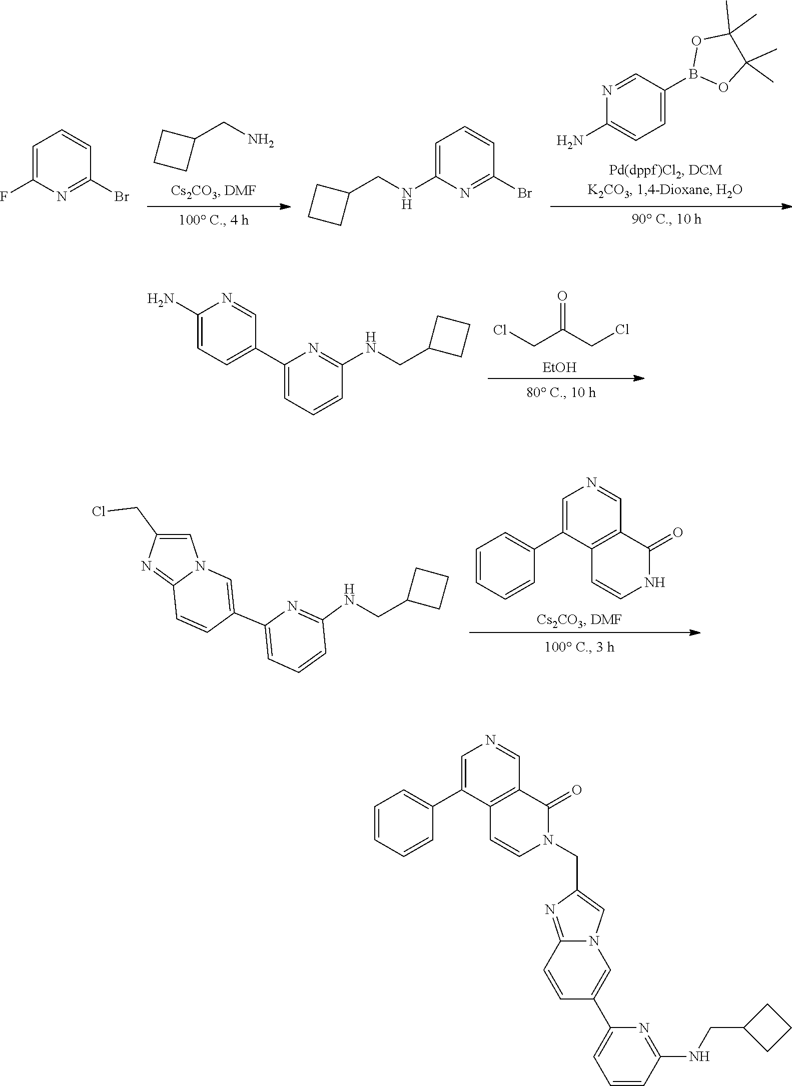

Step 1: Caesium carbonate (2.73 g, 0.0084 mmol) was added to a stirred solution of 2-bromo-6-fluoropyridine (500 mg, 0.0028 mmol) in DMF (10 mL), followed by the addition of cyclobutylmethanamine (357 mg, 0.0042 mmol). The reaction mixture was heated at 100° C. for 4 h, cooled to room temperature, poured into water (10 mL), and extracted with 10% methanol in DCM (20 mL×2). The combined organic layers were dried over sodium sulphate, filtered and concentrated in vacuo to give 6-bromo-N-(cyclobutylmethyl)pyridin-2-amine (220 mg, yield: 29%). MS: m/z: 242 [M+H]⁺

Step 2: A solution of 6-bromo-N-(cyclobutylmethyl)pyridin-2-amine (241 mg, 1 mmol) in a mixture of 1,4-dioxane and water (4:1)(10 mL) was purged with argon for 20 min, followed by the addition of K₂CO₃ (251 mg, 1.82 mmol), 5-(4,4,5,5-tetramethyl-1,3,2-dioxaborolan-2-yl)pyridin-2-amine (0.2 g 909 μmol) and Pd(dppf)Cl₂.DCM complex (148 mg, 182 μmol), and heated at 90° C. for 10 h. The reaction mixture was cooled to room temperature, diluted with EtOAc (30 mL), filtered through a celite pad, and the filtrate was concentrated in vacuo and purified by mPLC (C18, 10% ACN/H2O) to afford N6-(cyclobutylmethyl)-[2,3′-bipyridine]-6,6′-diamine (90 mg, yield: 79%) as a pale brown solid. MS: m/z: 255 [M+H]⁺

Step 3: The intermediate compound was prepared using the procedure described for example 1, step 1 with the following modification: the reaction was performed with N6-(cyclobutylmethyl)-[2,3′-bipyridine]-6,6′-diamine (90 mg, 354 μmol) and purified by mPLC (C18, 15% ACN/H₂O) to afford 6-[2-(chloromethyl)imidazo[1,2-a]pyridin-6-yl]-N-(cyclobutylmethyl)pyridin-2-amine (35 mg, yield: 30%) as an off-white solid.

Step 4: The title compound was prepared using the procedure described for example 1, step 2 with the following modification: the reaction was performed with 5-phenyl-1,2-dihydro-2,7-naphthyridin-1-one (37.4 mg, 168 μmol) and 6-[2-(chloromethyl)imidazo[1,2-a]pyridin-6-yl]-N-(cyclobutylmethyl)pyridin-2-amine (55 mg, 168 μmol) and purified by reverse phase prep-HPLC (Column: YMC Triat C18 (250*21.2 mm)5 μ, 0-95% CH₃CN/H₂O) to afford 2-[(6-{6-[(cyclobutylmethyl)amino]pyridin-2-yl}imidazo[1,2-a]pyridin-2-yl)methyl]-5-phenyl-1,2-dihydro-2,7-naphthyridin-1-one (5.14 mg, yield: 6%) as an off-white solid. ¹H NMR (400 MHz, DMSO-d₆): δ 9.41 (s, 1H), 9.13 (s, 1H), 8.67 (s, 1H), 7.99 (s, 1H), 7.92-7.84 (m, 2H), 7.59-7.49 (m, 5H), 7.43 (dd, J=8.4, 7.2 Hz, 1H), 7.00 (d, J=7.2 Hz, 1H), 6.60 (t, J=5.6 Hz, 1H), 6.49 (d, J=7.6 Hz, 1H), 6.44 (d, J=8.4 Hz, 1H), 5.33 (s, 2H), 3.37 (dd, J=7.2, 5.6 Hz, 2H), 2.57-2.52 (m, 1H), 2.06-1.97 (m, 2H), 1.93-1.80 (m, 2H), 1.75-1.68 (m, 2H). MS: m/z: 518 [M+H]⁺

### Synthetic Example 105: 2-((6-(4-(azetidin-1-yl)pyridin-2-yl)imidazo[1,2-a]pyridin-2-yl)methyl)-5-phenyl-2,7-naphthyridin-1(2H)-one. (Compound No. 105)

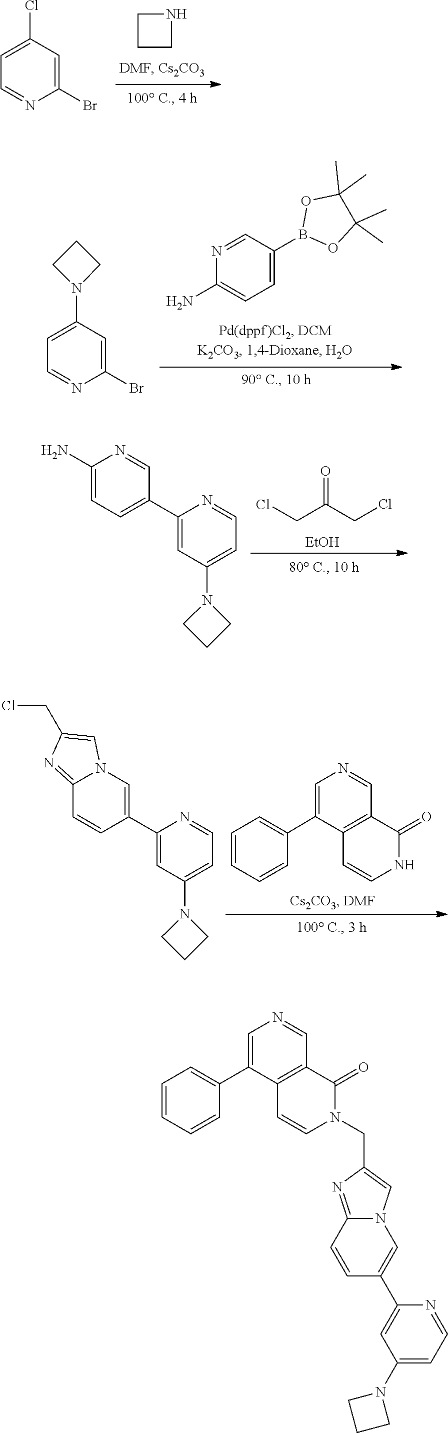

Step 1: The intermediate compound was prepared using the procedure described for example 104, step 1 with the following modification: the reaction was performed with 2-bromo-4-chloropyridine (500 mg, 0.0025 mmol) to give 4-(azetidin-1-yl)-2-bromopyridine (230 mg, yield: 42%). MS: m/z: 241 [M+H]⁺

Step 2: The intermediate compound was prepared using the procedure described for example 104, step 2 with the following modification: the reaction was performed with 4-(azetidin-1-yl)-2-bromopyridine(213 mg, 1 mmol) to give 4-(azetidin-1-yl)-[2,3′-bipyridin]-6′-amine (120 mg, yield: 59%) as a pale brown solid. MS: m/z: 227 [M+H]⁺

Step 3: The intermediate compound was prepared using the procedure described for example 104, step 3 with the following modification: the reaction was performed with 4-(azetidin-1-yl)-[2,3′-bipyridin]-6′-amine (70 mg, 309 μmol) to 4-(azetidin-1-yl)-2-[2-(chloromethyl)imidazo[1,2-a]pyridin-6-yl]pyridine (35 mg, yield: 37.9%) as a pale brown solid. MS: m/z: 299 [M+H]⁺

Step 4: The title compound was prepared using the procedure described for example 104, step 4 with the following modification: the reaction was performed with 5-phenyl-1,2-dihydro-2,7-naphthyridin-1-one (36.8 mg, 166 μmol) to give 2-({6-[4-(azetidin-1-yl)pyridin-2-yl]imidazo[1,2-a]pyridin-2-yl}methyl)-5-phenyl-1,2-dihydro-2,7-naphthyridin-1-one (10 mg, yield: 13%) as an off-white solid. ¹H NMR (400 MHz, DMSO-d₆): δ 9.40 (s, 1H), 9.22 (s, 1H), 8.67 (s, 1H), 8.19 (d, J=5.6 Hz, 1H), 7.98 (s, 1H), 7.92-7.87 (m, 2H), 7.58-7.49 (m, 6H), 6.84 (d, J=2.0 Hz, 1H), 6.49 (d, J=7.6 Hz, 1H), 6.32-6.30 (m, 1H), 5.33 (s, 2H), 3.99 (t, J=7.6 Hz, 4H), 2.42-2.37 (m, 2H). MS: m/z: 485 [M+H]⁺

### Synthetic Example 106: Synthesis of 3-((6-methylimidazo[1,2-a]pyridin-2-yl)methyl)pyrido[4,3-d]pyrimidin-4(3H)-one. (Compound No. 106)

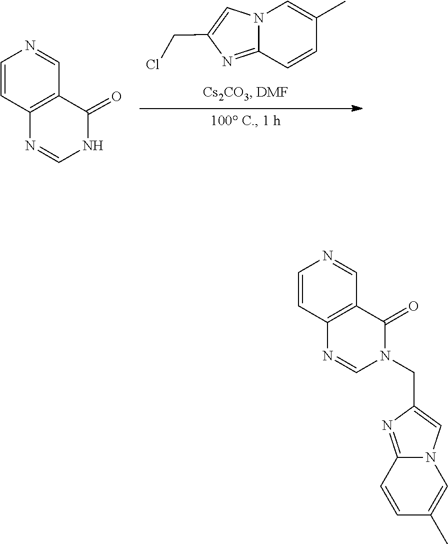 [SMILES: O=c1[nH]cnc2ccncc12 ; Cc1ccc2nc(CCl)cn2c1 ; Cc1ccc2nc(Cn3cnc4ccncc4c3=O)cn2c1]

The title compound was prepared using the procedure described for example 1, step 2 with the following modification: the reaction was performed with 3H, 4H-pyrido[4,3-d]pyrimidin-4-one (80.0 mg, 544 μmol) and purified by reverse phase prep-HPLC (YMC C18(250*20 MM*5 u, 0-95% CH₃CN/H₂O) to afford 3-({6-methylimidazo[1,2-a]pyridin-2-yl}methyl)-3H, 4H-pyrido[4,3-d]pyrimidin-4-one (18.8 mg, yield: 12%) as an off white solid. ¹H NMR (400 MHz, DMSO-d₆): δ 9.31 (s, 1H), 8.85 (d, J=5.6 Hz, 1H), 8.77 (s, 1H), 8.29 (s, 1H), 7.85 (s, 1H), 7.62 (d, J=5.6 Hz, 1H), 7.39 (d, J=9.2 Hz, 1H), 7.08 (dd, J=1.2, 9.2 Hz, 1H), 5.31 (s, 2H), 2.24 (s, 3H). MS: m/z: 292.1 [M+H]⁺

### Synthetic Example 107: Synthesis of 8-benzyl-3-((6-methylimidazo[1,2-a]pyridin-2-yl)methyl)pyrido[43-d]pyrimidin-4(3H)-one. (Compound No. 107)

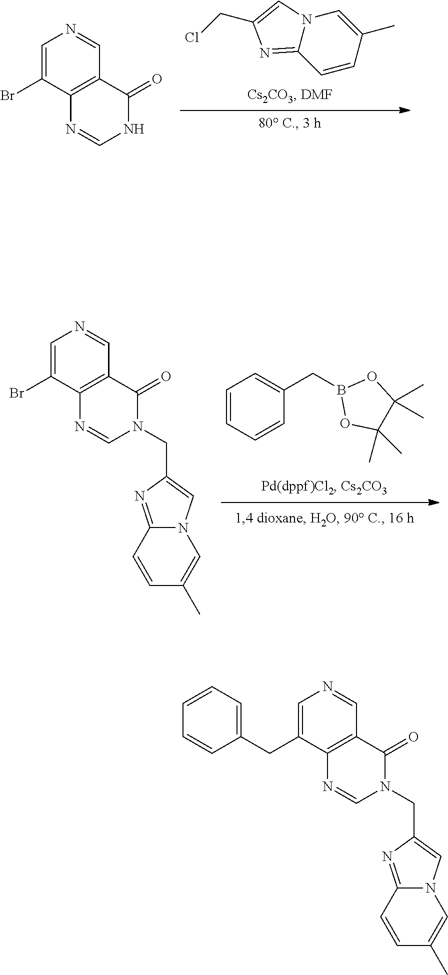 [SMILES: O=c1[nH]cnc2c(Br)cncc12 ; Cc1ccc2nc(CCl)cn2c1 ; Cc1ccc2nc(Cn3cnc4c(Br)cncc4c3=O)cn2c1 ; CC1(C)OB(Cc2ccccc2)OC1(C)C ; Cc1ccc2nc(Cn3cnc4c(Cc5ccccc5)cncc4c3=O)cn2c1]

Step 1: The intermediate compound was prepared using the procedure described for example 1, step 2 with the following modification: the reaction was performed with 8-bromo-3H, 4H-pyrido[4,3-d]pyrimidin-4-one (125 mg, 554 μmol) to give 8-bromo-3-((6-methylimidazo[1,2-a]pyridin-2-yl)methyl)pyrido[4,3-d]pyrimidin-4(3H)-one (60 mg, yield: 29%) as a brown solid.

Step 2: The title compound was prepared using the procedure described for example 104, step 2 with the following modification: the reaction was performed with 8-bromo-3-({6-methylimidazo[1,2-a]pyridin-2-yl}methyl)-3H, 4H-pyrido[4,3-d]pyrimidin-4-one (60.0 mg, 162 μmol) and 2-benzyl-4,4,5,5-tetramethyl-1,3,2-dioxaborolane (35.3 mg, 162 μmol), and purified by reverse phase Prep HPLC (Column: YMC Triart C18 (250*20 mm)5μ 0-95% CH₃CN/H₂O) to give rise to 8-benzyl-3-({6-methylimidazo[1,2-a]pyridin-2-yl}methyl)-3H, 4H-pyrido[4,3-d]pyrimidin-4-one (8.0 mg, yield: 20%) as an off-white solid. ¹H NMR (400 MHz, DMSO-d₆): δ 9.19 (s, 1H), 8.79 (d, J=8.4 Hz, 2H), 8.29 (s, 1H), 7.86 (s, 1H), 7.40 (d, J=9.2 Hz, 1H), 7.32-7.24 (m, 4H), 7.19-7.16 (m, 1H), 7.08 (d, J=9.2, 1.6 Hz, 1H), 5.30 (s, 2H), 4.32 (s, 2H), 2.24 (s, 3H). MS: m/z: 382.2 [M+H]⁺

### Synthetic Example 108: Synthesis of 3-((6-methylimidazo[1,2-a]pyridin-2-yl)methyl)-8-phenylpyrido[4,3-d]pyrimidin-4(3H)-one. (Compound No. 108)

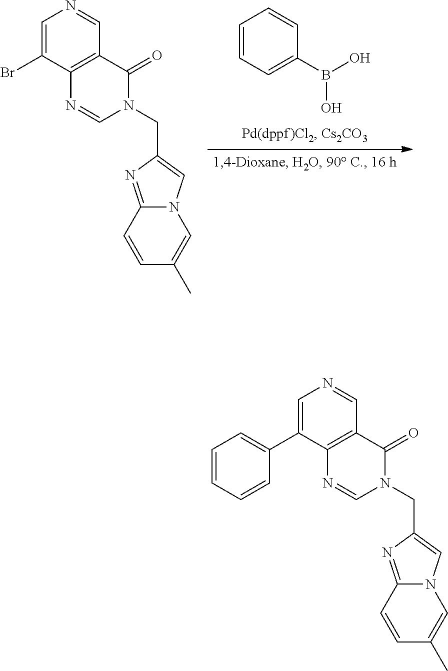 [SMILES: Cc1ccc2nc(Cn3cnc4c(Br)cncc4c3=O)cn2c1 ; OB(O)c1ccccc1 ; Cc1ccc2nc(Cn3cnc4c(-c5ccccc5)cncc4c3=O)cn2c1]

The title compound was prepared using the procedure described for example 107, step 2 with the following modification: the reaction was performed with phenyl boronic acid to give 3-({6-methylimidazo[1,2-a]pyridin-2-yl}methyl)-8-phenyl-3H, 4H-pyrido[4,3-d]pyrimidin-4-one (14 mg, yield: 14%) as an off white solid. ¹H NMR (400 MHz, DMSO-d₆): δ 9.33 (s, 1H), 8.90 (s, 1H), 8.75 (s, 1H), 8.46 (s, 1H), 8.07 (s, 1H), 7.66 (dd, J=8.4, 1.6 Hz, 2H), 7.60-7.58 (m, 1H), 7.55-7.45 (m, 3H), 7.44-7.39 (m, 1H), 5.39 (s, 2H), 2.31 (s, 3H). MS: m/z: 368 [M+H]⁺

### Synthetic Example 109: Synthesis of 8-cyclopropyl-3-((6-methylimidazo[1,2-a]pyridin-2-yl)methyl)pyrido[4,3-d]pyrimidin-4(3H)-one. (Compound No. 109)

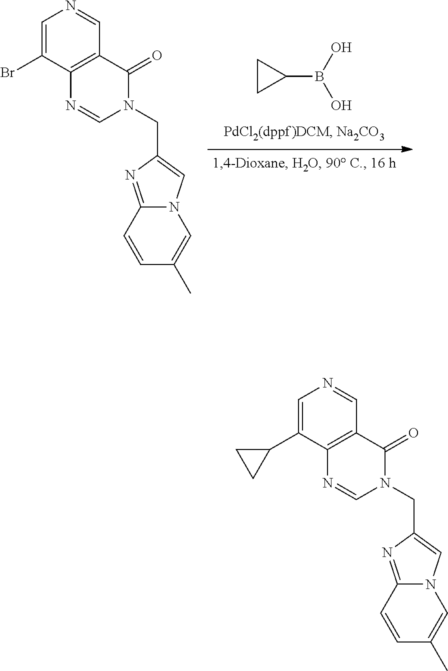 [SMILES: Cc1ccc2nc(Cn3cnc4c(Br)cncc4c3=O)cn2c1 ; OB(O)C1CC1 ; Cc1ccc2nc(Cn3cnc4c(C5CC5)cncc4c3=O)cn2c1]

The title compound was prepared using the procedure described for example 107, step 2 with the following modification: the reaction was performed with cyclopropyl boronic acid to give 8-cyclopropyl-3-({6-methylimidazo[1,2-a]pyridin-2-yl}methyl)-3H, 4H-pyrido[4,3-d]pyrimidin-4-one (yield: 9%) as an off white solid. ¹H NMR (400 MHz, DMSO-d₆): δ 9.10 (s, 1H), 8.79 (s, 1H), 8.43 (s, 1H), 8.29 (s, 1H), 7.85 (s, 1H), 7.40 (d, J=9.2 Hz, 1H), 7.09 (dd, J=9.2, 1.6 Hz, 1H), 6.28 (s, 1H), 5.31 (s, 2H), 2.67-2.63 (m, 1H), 2.24 (s, 3H), 1.11-1.06 (m, 2H), 0.99-0.96 (m, 2H). MS: m/z: 332 [M+H]⁺

### Synthetic Example 110: Synthesis of 2-((6-((((3-fluorocyclobutyl)methyl)amino)methyl)imidazo [1,2-a]pyridin-2-yl)methyl)-5-(2-azaspiro[3.3]heptan-2-yl)-2,7-naphthyridin-1(2H)-one. (Compound No. 110)

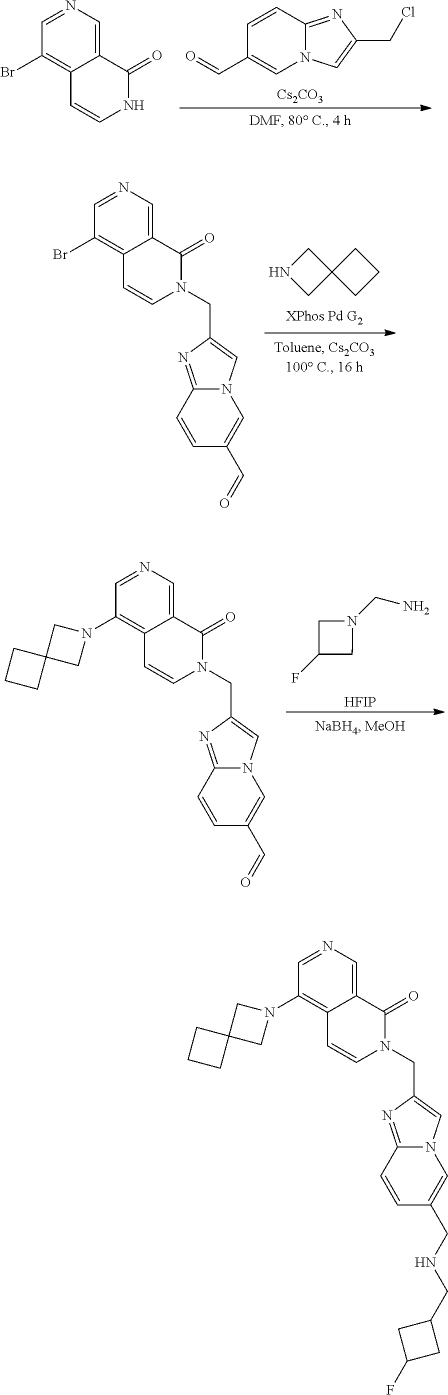

Step 1: Caesium carbonate(869 mg, 2.67 mmol) was added to a solution of 5-bromo-1,2-dihydro-2,7-naphthyridin-1-one (0.2 g, 889 μmol) and 2-(chloromethyl)imidazo[1,2-a]pyridine-6-carbaldehyde (208 mg, 1.07 mmol) in DMF (6 mL) and the reaction mixture was heated at 80° C. for 4 h. After cooling to room temperature, the reaction mixture was diluted with cold water (20 mL), and the precipitated solid was filtered and dried to give 2-[(5-bromo-1-oxo-1,2-dihydro-2,7-naphthyridin-2-yl)methyl]imidazo[1,2-a]pyridine-6-carbaldehyde (180 mg, yield: 52%) as a white solid. MS: m/z: 383 [M+H]⁺

Step 2: A solution of 2-[(5-bromo-1-oxo-1,2-dihydro-2,7-naphthyridin-2-yl)methyl]imidazo[1,2-a]pyridine-6-carbaldehyde (80 mg, 209 μmol), caesium carbonate (204 mg, 626 μmol) and 2-azaspiro[3.3]heptane (24.3 mg, 251 μmol) in toluene (10 mL) was purged with argon for 10 min, followed by the addition of XPhos-Pd-G2(32.9 mg, 41.8 μmol). The reaction mixture was heated at 100° C. for 16 h, cooled to room temperature and filtered through a celite pad. The filtrate was concentrated in vacuo and the residue was triturated with diethyl ether (10 mL) to give 2-[(5-{2-azaspiro[3.3]heptan-2-yl}-1-oxo-1,2-dihydro-2,7-naphthyridin-2-yl)methyl]imidazo[1,2-a]pyridine-6-carbaldehyde (38 mg, yield: 45%) as a gummy solid. MS: m/z: 400.3 [M+H]⁺

Step 3: 1-(3-fluorocyclobutyl)methanamine (10.8 mg, 105 μmol) was added to a stirred solution of 2-[(5-{2-azaspiro[3.3]heptan-2-yl}-1-oxo-1,2-dihydro-2,7-naphthyridin-2-yl)methyl]imidazo[1,2-a]pyridine-6-carbaldehyde (35 mg, 87.6 μmol) in HFIP (3 mL) at room temperature and stirred for 16 h. Sodium borohydride (6.63 mg, 175 μmol) and a catalytic amount of methanol (0.3 mL) were added to the reaction mixture and stirred for additional 2 h. The reaction mixture was diluted with ice cold water (15 mL), extracted with ethyl acetate (2×30 mL), concentrated in vacuo and the residue was purified by reverse phase prep HPLC method (Column: GEMINI C18 2.0*50 mm, 3 μm: 0-95% CH₃CN/H₂O) to give 5-{2-azaspiro[3.3]heptan-2-yl}-2-{[6-({[(3-fluorocyclobutyl)methyl]amino}methyl)imidazo[1,2-a]pyridin-2-yl]methyl}-1,2-dihydro-2,7-naphthyridin-1-one (10 mg, yield: 24%) as an off white solid. [0682]¹H NMR (400 MHz, DMSO-d₆): δ 8.78 (s, 1H), 8.36 (s, 1H), 7.85 (s, 1H), 7.82 (s, 1H), 7.68 (d, J=7.6 Hz, 1H), 7.43 (d, J=9.2 Hz, 1H), 7.22 (dd, J=9.2, 1.6 Hz, 1H), 6.58 (d, J=8.0 Hz, 1H), 5.24 (s, 2H), 5.15 (p, J=6.0 Hz, 0.5H), 5.01 (p, J=6.0 Hz, 0.5H), 4.10 (s, 4H), 3.63 (d, J=4.0 Hz, 2H), 2.47 (d, J=7.2 Hz, 2H), 2.38-2.35 (m, 1H), 2.20 (t, J=7.2 Hz, 6H), 2.16-2.07 (m, 2H), 1.85-1.77 (m, 2H). MS: m/z: 487.4 [M+H]⁺

### Synthetic Example 111: Synthesis of 5-{6,6-difluoro-2-azaspiro[3.3]heptan-2-yl}-2-({6-[(4,4-dimethylpiperidin-1-yl)methyl]imidazo[1,2-a]pyridin-2-yl}methyl)-1,2-dihydro-2,7-naphthyridin-1-one. (Compound No. 111)

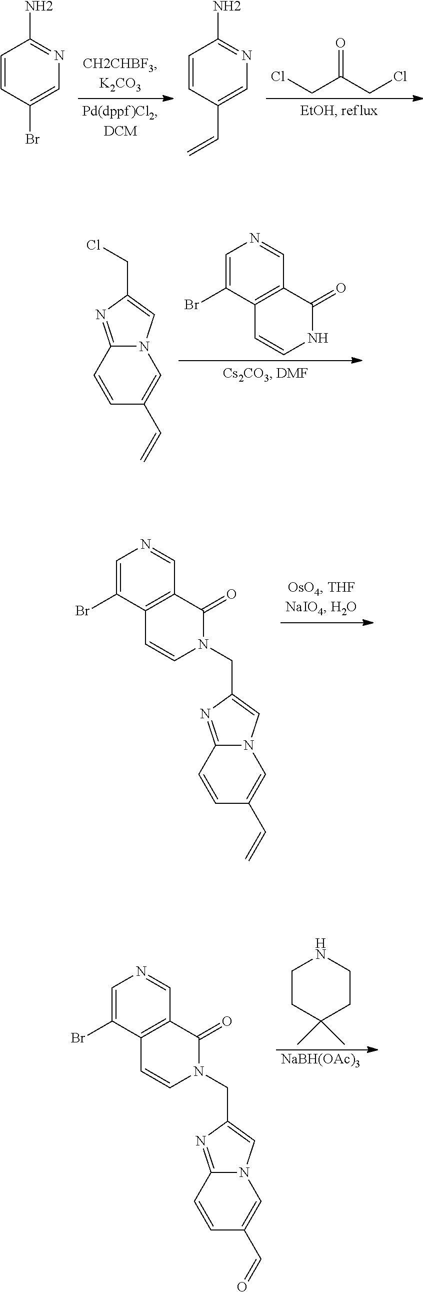

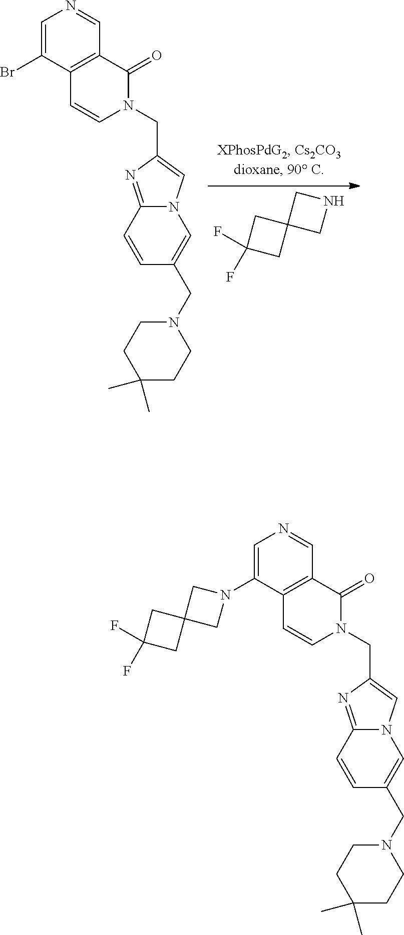

Step 1: A stirred solution of 5-bromopyridin-2-amine (5 g, 28.9 mmol), potassium ethenyltrifluoroboranuide (11.6 g 86.7 mmol) and K₂CO₃ (31 g 232 mmol) in EtOH (100 mL) was purged for 10 min with nitrogen. PdCl₂(dppf)DCM (2.36 g, 2.89 mmol) was then added and the reaction mixture was heated at 90° C. for 16 h under an atmosphere of nitrogen. The reaction mixture was cooled to room temperature and partitioned between EtOAc (100 mL×2) and water (100 mL). The combined organic layers were dried over Na₂SO₄, filtered, and concentrated in vacuo to afford 5-vinylpyridin-2-amine (3.47 g, yield: 99%). ¹H NMR (400 MHz, DMSO-d6) δ=7.92 (s, 1H), 7.60-7.55 (m, 1H), 6.60-6.49 (m, 1H), 6.43 (d, J=8.7 Hz, 1H), 6.06 (br s, 2H), 5.53 (d, J=17.6 Hz, 1H), 4.99 (d, J=11.2 Hz, 1H); MS: m/z 121.10 [M+H]⁺

Step 2: The intermediate compound was prepared using the procedure described for example 1, step 1 with the following modification: the reaction was performed with 5-ethenylpyridin-2-amine to give 2-(chloromethyl)-6-ethenylimidazo[1,2-a]pyridine (yield: 32%) as a brown solid. ¹H NMR (400 MHz, DMSO-d6) δ=8.57 (s, 1H), 7.97 (s, 1H), 7.62-7.55 (m, 1H), 7.54-7.48 (m, 1H), 6.71 (dd, J=17.6, 11.0 Hz, 1H), 5.86 (d, J 17.6 Hz, 1H), 5.33 (d, J=11.8 Hz, 1H), 4.84 (s, 2H); MS: m/z 192.90 [M+H]⁺

Step 3: The intermediate compound was prepared using the procedure described for example 1, step 2 with the following modification: the reaction was performed with 2-(chloromethyl)-6-ethenylimidazo[1,2-a]pyridine and 5-bromo-1,2-dihydro-2,7-naphthyridin-1-one to give 5-bromo-2-({6-ethenylimidazo[1,2-a]pyridin-2-yl}methyl)-1,2-dihydro-2,7-naphthyridin-1-one (yield: 56%) as a brown solid. ¹H NMR (400 MHz, DMSO-d6) δ=9.29 (s, 1H), 8.94 (s, 1H), 8.52 (s, 1H), 8.04 (d, J=7.6 Hz, 1H), 7.87 (s, 1H), 7.53 (dd, J=9.5, 1.6 Hz, 1H), 7.50-7.45 (m, 1H), 6.71 (d, J=8.1 Hz, 1H), 5.83 (d, J=17.6 Hz, 1H), 5.31 (s, 2H); MS: m/z 380.95 [M+H]⁺

Step 4: OSO₄, 4% in H₂O (660 μL) was added to a stirred solution of 5-bromo-2-({6-ethenylimidazo[1,2-a]pyridin-2-yl}methyl)-1,2-dihydro-2,7-naphthyridin-1-one (1 g, 2.62 mmol) in THF:H2O (8:2)(100 mL) at 0° C., and after 1 h, sodium periodate (2.81 g, 13.1 mmol) was added. The reaction mixture was allowed to warm to room temperature, stirred for an additional 16 h, and diluted with ice cold H₂O (50 mL). The solid obtained was filtered and washed with diethyl ether to give 2-[(5-bromo-1-oxo-1,2-dihydro-2,7-naphthyridin-2-yl)methyl]imidazo[1,2-a]pyridine-6-carbaldehyde(955 mg, yield: 85%) as an off white solid. ¹H NMR (300 MHz, DMSO-d6) δ=9.90 (s, 1H), 9.28 (s, 1H), 9.26 (s, 1H), 8.95 (s, 1H), 8.12 (s, 1H), 8.07 (d, J=7.7 Hz, 1H), 7.59 (s, 1H), 7.58 (br s, 1H), 6.73 (d, J=7.4 Hz, 1H), 5.36 (s, 2H); MS (m/z): 384.70 [M+H]⁺

Step 5: To a stirred solution of 2-[(5-bromo-1-oxo-1,2-dihydro-2,7-naphthyridin-2-yl)methyl]imidazo[1,2-a]pyridine-6-carbaldehyde(120 mg, 313 μmol) and 4,4-dimethylpiperidine (106 mg, 939 μmol) in DCE (4.3 mL) was added DIPEA(243 mg, 1.88 mmol) and NaBH(OAc)₃ (398 mg, 1.88 mmol) at room temperature. The reaction mixture was heated at 80° C. for 4 h, cooled to room temperature, diluted with water (20 ml), and extracted by 10% MeOH in DCM (3×50 ml). The combined organic layers were washed with brine solution, dried over Na₂SO₄, filtered, concentrated in vacuo and purified by flash column chromatography (SiO₂, 5%- 15% MeOH in DCM) to give 5-bromo-2-({6-[(4,4-dimethylpiperidin-1-yl)methyl]imidazo[1,2-a]pyridin-2-yl}methyl)-1,2-dihydro-2,7-naphthyridin-1-one(45 mg, yield: 30%) as an off white solid. ¹H NMR (400 MHz, DMSO-d6) δ=9.29 (s, 1H), 8.94 (s, 1H), 8.37 (s, 1H), 8.04 (d, J=7.5 Hz, 1H), 7.86 (s, 1H), 7.43 (br d, J=8.9 Hz, 1H), 7.17 (br d, J=9.1 Hz, 1H), 6.71 (d, J=7.6 Hz, 1H), 5.30 (s, 2H), 3.42 (s, 2H), 2.36-2.31 (m, 4H), 1.31 (br d, J=4.8 Hz, 4H), 0.88 (s, 6H); MS: m/z 479.90 [M+H]⁺

Step 6: A solution of 5-bromo-2-({6-[(4,4-dimethylpiperidin-1-yl)methyl]imidazo[1,2-a]pyridin-2-yl}methyl)-1,2-dihydro-2,7-naphthyridin-1-one (50 mg, 104 μmol), 6,6-difluoro-2-azaspiro[3.3]heptane (71.5 mg, 312 μmol) and Cs₂CO₃ (102 mg, 312 μmol) in toluene (5 mL) was purged with nitrogen for 10 min, followed by the addition of XPhosPd-G2 (8.1 mg, 10.4 μmol). The reaction mixture was heated at 100° C. for 2 h, cooled to room temperature and filtered through a celite pad. The filtrate was diluted with water (10 ml), and extracted with 10% MeOH in DCM (3×20 ml). The combined organic layers were washed with brine solution, dried over Na₂SO₄, filtered, concentrated in vacuo and purified by preparative TLC (SiO₂, 5% MeOH in DCM) to give 5-{6,6-difluoro-2-azaspiro[3.3]heptan-2-yl}-2-({6-[(4,4-dimethylpiperidin-1-yl)methyl]imidazo[1,2-a]pyridin-2-yl}methyl)-1,2-dihydro-2,7-naphthyridin-1-one(9.5 mg, yield: 16%) as an off white solid. ¹H NMR (400 MHz, DMSO-d6) δ=8.82 (s, 1H), 8.37 (s, 1H), 7.90 (s, 1H), 7.82 (s, 1H), 7.71 (d, J=7.8 Hz, 1H), 7.42 (d, J=9.2 Hz, 1H), 7.17 (d, J=9.2 Hz, 1H), 6.57 (d, J=7.8 Hz, 1H), 5.25 (s, 2H), 4.22 (s, 4H), 3.41 (s, 2H), 2.88 (t, J=12.5 Hz, 4H), 2.35-2.30 (m, 4H), 1.30 (t, J=5.5 Hz, 4H), 0.87 (s, 6H); MS: m/z 533.00 [M+H]⁺

### Synthetic Example 112: Synthesis of 5-(4,4-difluoropiperidin-1-yl)-2-({6-[(4,4-dimethylpiperidin-1-yl)methyl]imidazo[1,2-a]pyridin-2-yl}methyl)-1,2-dihydro-2,7-naphthyridin-1-one (Compound No. 112)

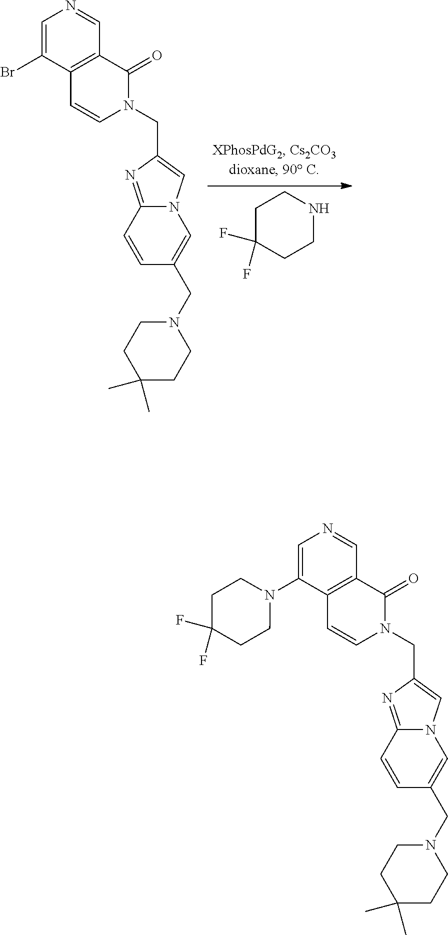

The title compound was prepared using the procedure described for example 111, step 6 with the following modification: the reaction was performed with 4,4-difluoropiperidine to give 5-(4,4-difluoropiperidin-1-yl)-2-({6-[(4,4-dimethylpiperidin-1-yl)methyl]imidazo[1,2-a]pyridin-2-yl}methyl)-1,2-dihydro-2,7-naphthyridin-1-one (yield: 69%) as an off-white solid. ¹H NMR (400 MHz, DMSO-d6) δ=9.07 (s, 1H), 8.44 (s, 1H), 8.38 (br s, 2H), 8.35 (s, 1H), 7.84 (d, J=7.6 Hz, 1H), 7.81 (s, 1H), 7.42 (d, J=9.2 Hz, 1H), 7.18 (d, J=9.2 Hz, 1H), 6.71 (d, J=7.6 Hz, 1H), 5.27 (s, 2H), 3.41 (s, 2H), 3.14 (br t, J=5.3 Hz, 4H), 2.34-2.29 (m, 2H), 2.25-2.11 (m, 4H), 2.04-1.91 (m, 2H), 1.35-1.26 (m, 4H), 0.86 (s, 6H); MS: m/z 521.20 [M+H]⁺

### Synthetic Example 113: Synthesis of 2-({6-[(4,4-dimethylpiperidin-1-yl)methyl]imidazo[1,2-a]pyridin-2-yl}methyl)-5-(morpholin-4-yl)-1,2-dihydro-2,7-naphthyridin-1-one (Compound No. 113)

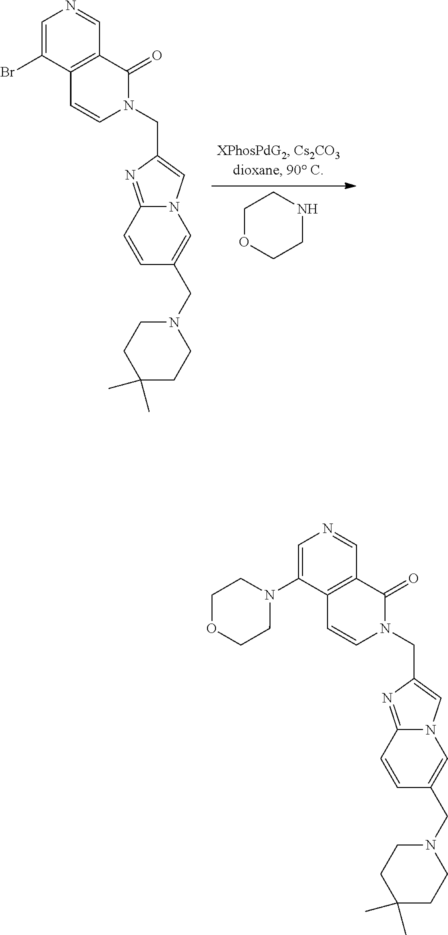 [SMILES: CC1(C)CCN(Cc2ccc3nc(Cn4ccc5c(Br)cncc5c4=O)cn3c2)CC1 ; C1COCCN1 ; CC1(C)CCN(Cc2ccc3nc(Cn4ccc5c(N6CCOCC6)cncc5c4=O)cn3c2)CC1]

The title compound was prepared using the procedure described for example 111, step 6 with the following modification: the reaction was performed with morpholine to give 2-({6-[(4,4-dimethylpiperidin-1-yl)methyl]imidazo[1,2-a]pyridin-2-yl}methyl)-5-(morpholin-4-yl)-1,2-dihydro-2,7-naphthyridin-1-one (yield: 33%) as a white solid. ¹H NMR (400 MHz, DMSO-d6) δ=9.08 (s, 1H), 8.42 (s, 1H), 8.37 (s, 1H), 7.84 (d, J=7.5 Hz, 1H), 7.82 (s, 1H), 7.42 (d, J=9.5 Hz, 1H), 7.17 (br d, J=9.1 Hz, 1H), 6.70 (d, J=7.8 Hz, 1H), 5.28 (s, 2H), 3.84-3.76 (m, 4H), 3.41 (s, 2H), 3.08-2.99 (m, 4H), 2.33-2.28 (m, 4H), 1.32-1.27((m, 4H), 0.88 (s, 6H); MS: m/z 486.95 [M+H]⁺

### Synthetic Example 114: Synthesis of 2-({6-[(4,4-dimethylpiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one (Compound No. 114)

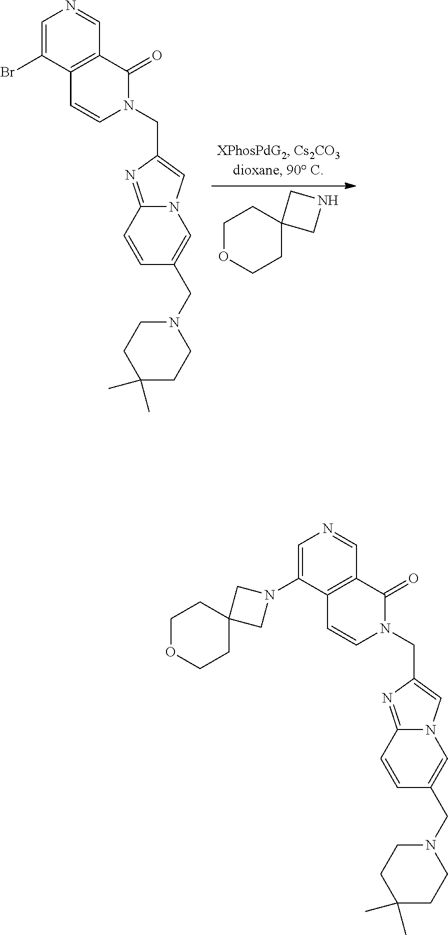

The title compound was prepared using the procedure described for example 111, step 6 with the following modification: the reaction was performed with 7-oxa-2-azaspiro[3.5]nonane to give 2-({6-[(4,4-dimethylpiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one (yield: 55%) as a pale brown solid. ¹H NMR (400 MHz, DMSO-d6) δ=8.78 (s, 1H), 8.37 (s, 1H), 7.88 (s, 1H), 7.81 (s, 1H), 7.68 (d, J=7.6 Hz, 1H), 7.42 (d, J=9.2 Hz, 1H), 7.19-7.14 (m, 1H), 6.63 (d, J=7.6 Hz, 1H), 5.25 (s, 2H), 3.93 (s, 4H), 3.62-3.50 (m, 4H), 3.41 (s, 2H), 2.33 (br s, 4H), 1.82-1.72 (m, 4H), 1.30 (br t, J=5.4 Hz, 4H), 0.87 (s, 6H); MS: m/z 527.05 [M+H]⁺

### Synthetic Example 115: Synthesis of 5-((1S,4S)-2-oxa-5-azabicyclo[2.2.1]heptan-5-yl)-2-((6-((4,4-dimethylpiperidin-1-yl)methyl)imidazo[1,2-a]pyridin-2-yl)methyl)-2,7-naphthyridin-1(2H)-one (Compound No. 115)

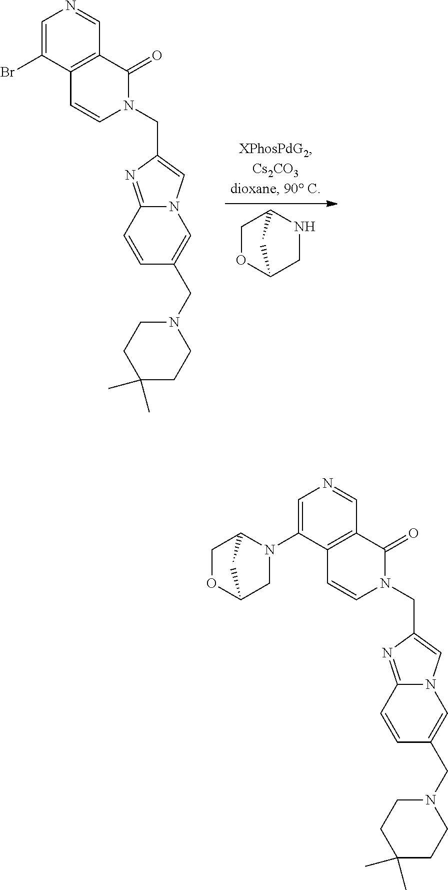

The title compound was prepared using the procedure described for example 111, step 6 with the following modification: the reaction was performed with (1S,4S)-2-oxa-5-azabicyclo[2.2.1]heptane to afford 5-((1S,4S)-2-oxa-5-azabicyclo[2.2.1]heptan-5-yl)-2-((6-((4,4-dimethylpiperidin-1-yl)methyl)imidazo[1,2-a]pyridin-2-yl)methyl)-2,7-naphthyridin-1(2H)-one (yield: 21%) as an off-white solid. ¹H NMR (400 MHz, METHANOL-d4) δ=8.97 (s, 1H), 8.34 (s, 1H), 8.22 (s, 1H), 7.82 (s, 1H), 7.67 (d, J=7.8 Hz, 1H), 7.49-7.44 (m, 1H), 7.36 (d, J=9.4 Hz, 1H), 6.83 (d, J=7.8 Hz, 1H), 5.35 (d, J=2.3 Hz, 2H), 4.64 (s, 1H), 4.51 (s, 1H), 4.09 (d, J=7.8 Hz, 1H), 3.89 (d, J=7.9 Hz, 1H), 3.79 (d, J=9.5 Hz, 1H), 3.38 (br d, J=9.8 Hz, 1H), 2.50 (br s, 4H), 2.14-2.06 (m, 1H), 1.99 (br d, J=10.0 Hz, 1H), 1.43 (br t, J=5.5 Hz, 4H), 0.94 (s, 6H); MS: m/z 499.15 [M+H]⁺

### Synthetic Example 116: Synthesis of 2-({6-[(4,4-dimethylpiperidin-1-yl)methyl]imidazo[1,2-a]pyridin-2-yl}methyl)-5-[(1R,4R)-2-oxa-5-azabicyclo[2.2.1]heptan-5-yl]-1,2-dihydro-2,7-naphthyridin-1-one (Compound No. 116)

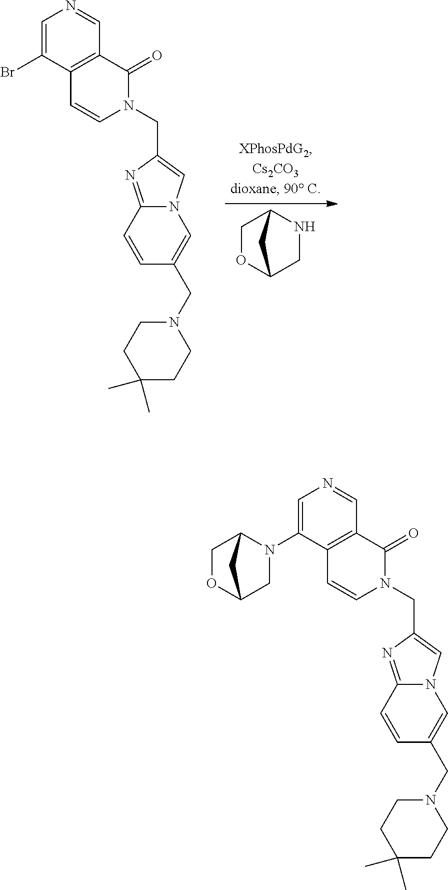 [SMILES: CC1(C)CCN(Cc2ccc3nc(Cn4ccc5c(Br)cncc5c4=O)cn3c2)CC1 ; C1O[C@H]2CN[C@@H]1C2 ; CC1(C)CCN(Cc2ccc3nc(Cn4ccc5c(N6C[C@H]7C[C@@H]6CO7)cncc5c4=O)cn3c2)CC1]

The title compound was prepared using the procedure described for example 111, step 6 with the following modification: the reaction was performed with (1R,4R)-2-oxa-5-azabicyclo[2.2.1]heptane to afford 2-({6-[(4,4-dimethylpiperidin-1-yl)methyl]imidazo[1,2-a]pyridin-2-yl}methyl)-5-[(1R,4R)-2-oxa-5-azabicyclo[2.2.1]heptan-5-yl]-1,2-dihydro-2,7-naphthyridin-1-one (yield: 22%) as a white solid. ¹H NMR (400 MHz, METHANOL-d4) δ=8.97 (s, 1H), 8.36 (s, 1H), 8.22 (s, 1H), 7.83 (s, 1H), 7.68 (d, J=7.8 Hz, 1H), 7.50-7.45 (m, 1H), 7.39-7.33 (m, 1H), 6.83 (d, J=7.8 Hz, 1H), 5.35 (d, J=2.9 Hz, 2H), 4.64 (s, 1H), 4.58 (s, 1H), 4.51 (s, 1H), 4.09 (d, J=7.8 Hz, 1H), 3.89 (d, J=7.6 Hz, 1H), 3.79 (d, J=9.5 Hz, 1H), 3.66-3.62 (m, 2H), 3.38-3.32 (m, 2H), 2.63-2.51 (m, 2H), 2.19 (t, J=7.6 Hz, 1H), 2.13-1.96 (m, 4H), 1.63-1.57 (m, 1H), 1.45 (br t, J=5.6 Hz, 2H), 0.95 (s, 6H); MS: m/z 498.95 [M+H]⁺

### Synthetic Example 117: Synthesis of 2-({6-[(4,4-dimethylpiperidin-1-yl)methyl]imidazo[1,2-a]pyridin-2-yl}methyl)-5-{hexahydro-1H-furo[3,4-c]pyrrol-5-yl}-1,2-dihydro-2,7-naphthyridin-1-one (Compound No. 117)

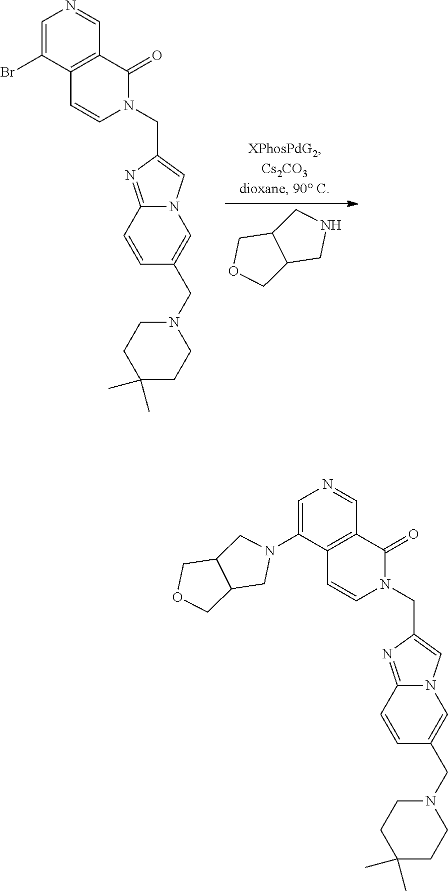 [SMILES: CC1(C)CCN(Cc2ccc3nc(Cn4ccc5c(Br)cncc5c4=O)cn3c2)CC1 ; C1NCC2COCC12 ; CC1(C)CCN(Cc2ccc3nc(Cn4ccc5c(N6CC7COCC7C6)cncc5c4=O)cn3c2)CC1]

The title compound was prepared using the procedure described for example 111, step 6 with the following modification: the reaction was performed with hexahydro-1H-furo[3,4-c]pyrrole to give 2-({6-[(4,4-dimethylpiperidin-1-yl)methyl]imidazo[1,2-a]pyridin-2-yl}methyl)-5-{hexahydro-1H-furo[3,4-c]pyrrol-5-yl}-1,2-dihydro-2,7-naphthyridin-1-one (yield: 53%) as a yellow sticky solid. ¹H NMR (400 MHz, DMSO-d6) δ=9.25 (br s, 1H), 9.02 (s, 1H), 8.63 (s, 1H), 8.33 (s, 1H), 8.07 (s, 1H), 7.90 (d, J=7.6 Hz, 1H), 7.66 (d, J=9.4 Hz, 1H), 7.41 (br d, J=9.7 Hz, 1H), 7.22 (s, 1H), 7.09 (s, 1H), 6.96 (s, 1H), 6.77 (d, J=7.6 Hz, 1H), 5.35 (s, 2H), 4.39-4.31 (m, 2H), 3.92-3.83 (m, 2H), 3.62-3.53 (m, 2H), 3.35-3.28 (m, 2H), 3.25-3.25 (m, 1H), 3.24 (br d, J=11.3 Hz, 1H), 3.15-3.10 (m, 2H), 3.09-3.03 (m, 2H), 3.01-2.94 (m, 2H), 1.56-1.47 (m, 4H), 1.02-0.97 (m, 3H), 0.94 (s, 3H); MS: m/z 513.80 [M+H]⁺

### Synthetic Example 118: Synthesis of 2-({6-[(4,4-dimethylpiperidin-1-yl)methyl]imidazo[1,2-a]pyridin-2-yl}methyl)-5-[3-(morpholin-4-yl)azetidin-1-yl]-1,2-dihydro-2,7-naphthyridin-1-one (Compound No. 118)

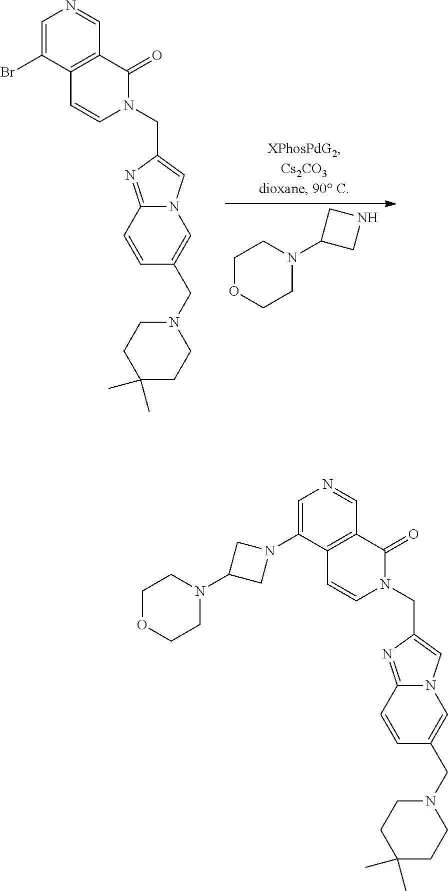

The title compound was prepared using the procedure described for example 111, step 6 with the following modification: the reaction was performed with 4-(azetidine)morpholine to give 2-((6-[(4,4-dimethylpiperidin-1-yl)methyl]imidazo[1,2-a]pyridin-2-yl)methyl)-5-[3-(morpholin-4-yl)azetidin-1-yl]-1,2-dihydro-2,7-naphthyridin-1-one(yield: 56%) as a pale yellow solid. ¹H NMR (400 MHz, DMSO-d6) δ=8.80 (s, 1H), 8.37 (s, 1H), 7.90 (s, 1H), 7.82 (s, 1H), 7.69 (d, J=7.8 Hz, 1H), 7.42 (d, J=9.2 Hz, 1H), 7.20-7.13 (m, 1H), 6.61 (d, J=7.6 Hz, 1H), 5.25 (s, 2H), 4.22 (t, J=7.3 Hz, 2H), 3.96-3.90 (m, 2H), 3.59 (br t, J=4.0 Hz, 4H), 3.41 (s, 2H), 3.30-3.22 (m, 1H), 2.40-2.29 (m, 8H), 1.30 (br t, J=5.4 Hz, 4H), 0.87 (s, 6H); MS: m/z 542.00 [M+H]⁺

### Synthetic Example 119: Synthesis of 5-{2,7-diazaspiro[3.5]nonan-2-yl}-2-({6-[(4,4-dimethylpiperidin-1-yl)methyl]imidazo[1,2-a]pyridin-2-yl}methyl)-1,2-dihydro-2,7-naphthyridin-1-one (Compound No. 119)

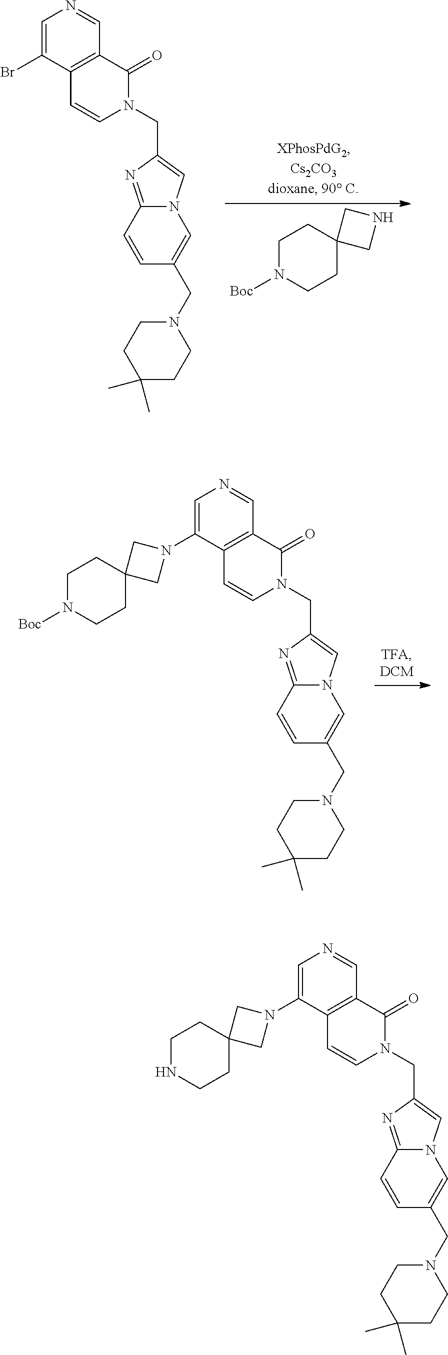 [SMILES: CC1(C)CCN(Cc2ccc3nc(Cn4ccc5c(Br)cncc5c4=O)cn3c2)CC1 ; CC(C)(C)OC(=O)N1CCC2(CC1)CNC2 ; CC1(C)CCN(Cc2ccc3nc(Cn4ccc5c(N6CC7(CCN(C(=O)OC(C)(C)C)CC7)C6)cncc5c4=O)cn3c2)CC1 ; CC1(C)CCN(Cc2ccc3nc(Cn4ccc5c(N6CC7(CCNCC7)C6)cncc5c4=O)cn3c2)CC1]

Step 1: The intermediate compound was prepared using the procedure described for example 111, step 6 with the following modification: the reaction was performed with tert-butyl 2,7-diazaspiro[3.5]nonane-7-carboxylate to give tert-butyl 2-[7-((6-[(4,4-dimethylpiperidin-1-yl)methyl]imidazo[1,2-a]pyridin-2-yl)methyl)-8-oxo-7,8-dihydro-2,7-naphthyridin-4-yl]-2,7-diazaspiro[3.5]nonane-7-carboxylate (yield: 47%) as a pale yellow solid. MS: m/z 626.25 [M+H]⁺

Step 2: Trifluoroacetic acid (0.2 mL) was added to a stirred solution of tert-butyl 2-[7-((6-[(4,4-dimethylpiperidin-1-yl)methyl]imidazo[1,2-a]pyridin-2-yl)methyl)-8-oxo-7,8-dihydro-2,7-naphthyridin-4-yl]-2,7-diazaspiro[3.5]nonane-7-carboxylate (35 mg, 55.9 μmol) in DCM (0.5 mL, 7.81 mmol) at 0° C., and the reaction mixture was allowed to warm to room temperature. After 2 h, the reaction mixture was concentrated in vacuo and purified by preparative HPLC (Column: Zorbax (250 mm×21.2 mm 5.0p, Mobile Phase: A=0.1% HCO₂H in H₂O, B=ACN) to afford 5-(2,7-diazaspiro[3.5]nonan-2-yl)-2-((6-[(4,4-dimethylpiperidin-1-yl)methyl]imidazo[1,2-a]pyridin-2-yl)methyl)-1,2-dihydro-2,7-naphthyridin-1-one(27 mg, yield: 92%) as a pale brown oil. ¹H NMR (300 MHz, DMSO-d6) δ=8.78 (s, 1H), 8.67 (s, 1H), 8.08 (s, 1H), 7.95 (d, J=7.7 Hz, 1H), 7.81 (s, 1H), 7.66 (br d, J=9.4 Hz, 1H), 7.50 (br d, J=9.4 Hz, 1H), 6.76 (br d, J=7.4 Hz, 1H), 5.36 (s, 2H), 4.33 (s, 2H), 4.03 (s, 4H), 3.21 (br d, J=11.8 Hz, 2H), 3.06 (br s, 6H), 2.01-1.87 (m, 4H), 1.59-1.43 (m, 4H), 0.97 (s, 3H), 0.93 (s, 3H); MS: m/z 525.80 [M+H]⁺

### Synthetic Example 120: Synthesis of 5-(3,4-dihydro-2H-1,4-benzoxazin-4-yl)-2-({6-[(4,4-dimethylpiperidin-1-yl)methyl]imidazo[1,2-a]pyridin-2-yl}methyl)-1,2-dihydro-2,7-naphthyridin-1-one (Compound 120)

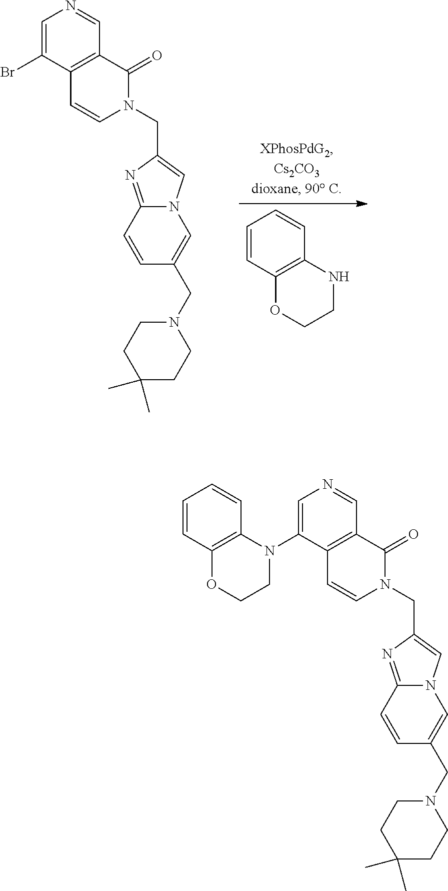 [SMILES: CC1(C)CCN(Cc2ccc3nc(Cn4ccc5c(Br)cncc5c4=O)cn3c2)CC1 ; c1ccc2c(c1)NCCO2 ; CC1(C)CCN(Cc2ccc3nc(Cn4ccc5c(N6CCOc7ccccc76)cncc5c4=O)cn3c2)CC1]

The title compound was prepared using the procedure described for example 111, step 6 with the following modification: the reaction was performed with 3,4-dihydro-2H-1,4-benzoxazine to afford 5-(3,4-dihydro-2H-1,4-benzoxazin-4-yl)-2-({6-[(4,4-dimethylpiperidin-1-yl)methyl]imidazo[1,2-a]pyridin-2-yl}methyl)-1,2-dihydro-2,7-naphthyridin-1-one (yield: 21%) as an orange sticky solid. ¹H NMR (400 MHz, DMSO-d6) δ=9.26 (s, 1H), 8.68 (s, 1H), 8.41-8.34 (m, 1H), 7.86 (s, 1H), 7.84 (s, 1H), 7.42 (d, J=9.2 Hz, 1H), 7.17 (d, J=9.2 Hz, 1H), 6.84 (d, J=7.8 Hz, 1H), 6.74-6.58 (m, 2H), 6.51 (d, J=7.5 Hz, 1H), 6.19-6.05 (m, 1H), 5.30 (s, 2H), 4.37 (s, 2H), 3.86-3.59 (m, 2H), 3.42 (s, 2H), 2.34-2.32 (m, 4H), 1.31 (br t, J=5.4 Hz, 4H), 0.88 (s, 6H); MS: m/z 535.00 [M+H]⁺

### Synthetic Example 121: Synthesis of 2-({6-[(4,4-dimethylpiperidin-1-yl)methyl]imidazo[1,2-a]pyridin-2-yl}methyl)-5-phenyl-1,2-dihydro-2,7-naphthyridin-1-one (Compound No. 121)

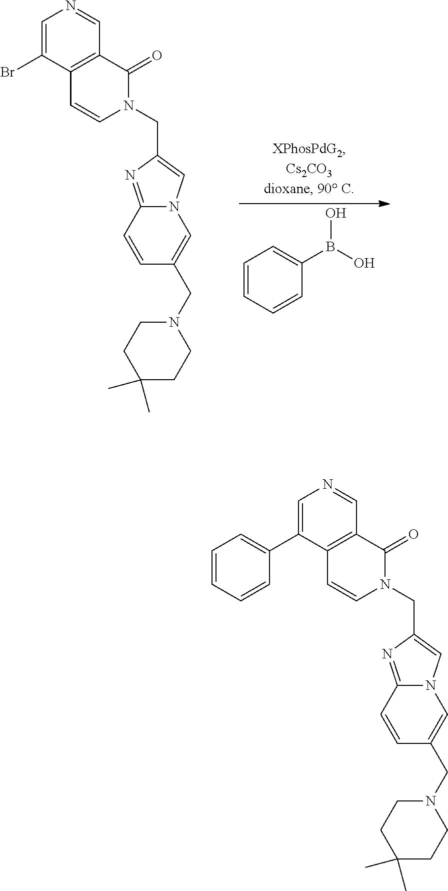 [SMILES: CC1(C)CCN(Cc2ccc3nc(Cn4ccc5c(Br)cncc5c4=O)cn3c2)CC1 ; OB(O)c1ccccc1 ; CC1(C)CCN(Cc2ccc3nc(Cn4ccc5c(-c6ccccc6)cncc5c4=O)cn3c2)CC1]

The title compound was prepared using the procedure described for example 111, step 6 with the following modification: the reaction was performed with phenylboronic acid to give 2-({6-[(4,4-dimethylpiperidin-1-yl)methyl]imidazo[1,2-a]pyridin-2-yl}methyl)-5-phenyl-1,2-dihydro-2,7-naphthyridin-1-one (yield: 13%) as an off white solid. ¹H NMR (400 MHz, DMSO-d6) δ=9.39 (s, 1H), 8.66 (s, 1H), 8.37 (s, 1H), 7.85 (t, J=3.7 Hz, 2H), 7.59-7.48 (m, 5H), 7.42 (d, J=9.2 Hz, 1H), 7.19-7.15 (m, 1H), 6.46 (d, J=7.8 Hz, 1H), 5.29 (s, 2H), 3.42 (s, 2H), 2.58-2.56 (m, 2H), 2.32-2.29 (m, 2H), 1.33-1.27((m, 4H), 0.88 (s, 6H); MS: m/z 478.31 [M+H]⁺

### Synthetic Example 122: Synthesis of 2-({6-[(4,4-dimethylpiperidin-1-yl)methyl]imidazo[1,2-a]pyridin-2-yl}methyl)-5-(quinolin-5-yl)-1,2-dihydro-2,7-naphthyridin-1-one (Compound No. 122)

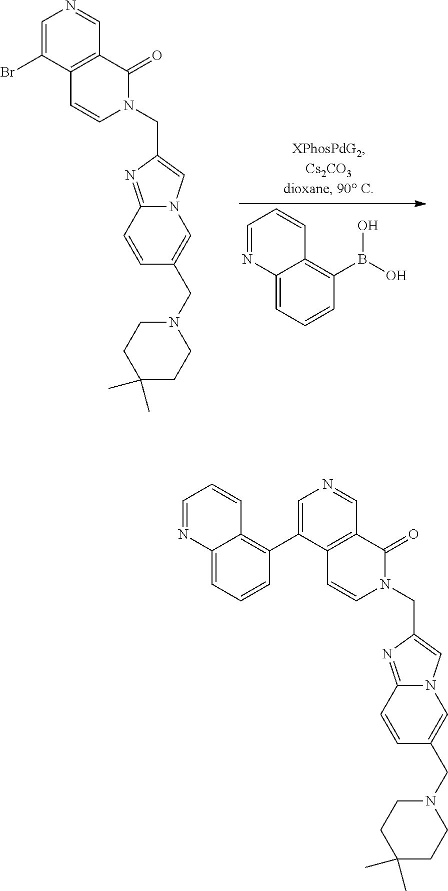 [SMILES: CC1(C)CCN(Cc2ccc3nc(Cn4ccc5c(Br)cncc5c4=O)cn3c2)CC1 ; OB(O)c1cccc2ncccc12 ; CC1(C)CCN(Cc2ccc3nc(Cn4ccc5c(-c6cccc7ncccc67)cncc5c4=O)cn3c2)CC1]

The title compound was prepared using the procedure described for example 111, step 6 with the following modification: the reaction was performed with (quinolin-5-yl)boronic acid to afford 2-({6-[(4,4-dimethylpiperidin-1-yl)methyl]imidazo[1,2-a]pyridin-2-yl}methyl)-5-(quinolin-5-yl)-1,2-dihydro-2,7-naphthyridin-1-one (yield: 33%) as a brown solid. ¹H NMR (400 MHz, DMSO-d6) δ=9.50 (s, 1H), 8.99-8.93 (m, 1H), 8.71 (s, 1H), 8.37 (s, 1H), 8.19 (d, J=8.5 Hz, 1H), 7.96-7.89 (m, 1H), 7.86 (s, 1H), 7.78-7.71 (m, 2H), 7.65-7.60 (m, 1H), 7.49-7.44 (m, 1H), 7.41 (d, J=9.2 Hz, 1H), 7.16 (d, J=7.9 Hz, 1H), 5.86 (d, J=7.5 Hz, 1H), 5.33-5.28 (m, 1H), 5.26-5.21 (m, 1H), 3.41 (s, 2H), 2.37-2.28 (m, 4H), 1.30 (br t, J=5.4 Hz, 4H), 0.88 (s, 6H); MS: m/z 529.10 [M+H]⁺

### Synthetic Example 123: Synthesis of 4-[7-({6-[(4,4-dimethylpiperidin-1-yl)methyl]imidazo[1,2-a]pyridin-2-yl}methyl)-8-oxo-7,8-dihydro-2,7-naphthyridin-4-yl]benzene-1-sulfonamide (Compound 123)

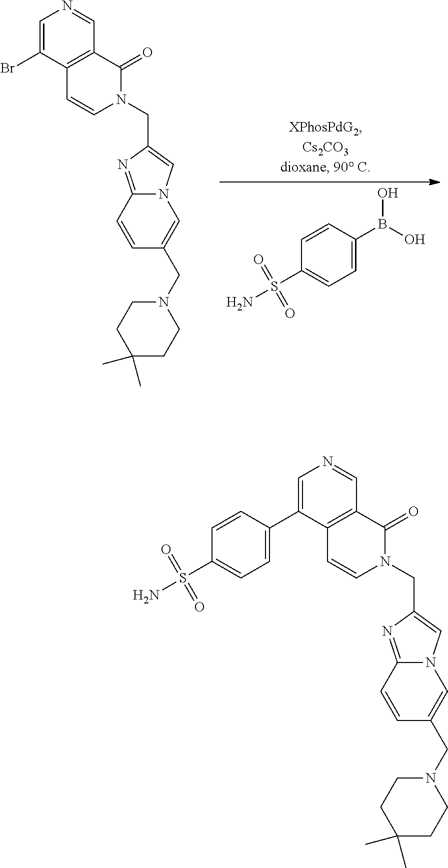 [SMILES: CC1(C)CCN(Cc2ccc3nc(Cn4ccc5c(Br)cncc5c4=O)cn3c2)CC1 ; NS(=O)(=O)c1ccc(B(O)O)cc1 ; CC1(C)CCN(Cc2ccc3nc(Cn4ccc5c(-c6ccc(S(N)(=O)=O)cc6)cncc5c4=O)cn3c2)CC1]

The title compound was prepared using the procedure described for example 111, step 6 with the following modification: the reaction was performed with (4-sulfamoylphenyl)boronic acid to afford 4-[7-({6-[(4,4-dimethylpiperidin-1-yl)methyl]imidazo[1,2-a]pyridin-2-yl}methyl)-8-oxo-7,8-dihydro-2,7-naphthyridin-4-yl]benzene-1-sulfonamide(yield: 52%) as a pale brown solid. ¹H NMR (400 MHz, DMSO-d6) δ=9.44 (s, 1H), 8.73 (s, 1H), 8.69 (s, 1H), 8.12 (s, 1H), 8.01 (d, J=8.4 Hz, 2H), 7.92 (d, J=7.6 Hz, 1H), 7.73 (s, 1H), 7.72 (d, J=6.7 Hz, 2H), 7.54 (d, J=10.6 Hz, 1H), 6.52 (d, J=7.6 Hz, 1H), 5.40 (s, 2H), 4.36 (s, 2H), 3.25 (br d, J=11.4 Hz, 2H), 3.13-3.02 (m, 2H), 1.59-1.48 (m, 4H), 1.00 (s, 3H), 0.96 (s, 3H); MS: m/z 557.10 [M+H]⁺

### Synthetic Example 124: Synthesis of 2-((6-((4,4-dimethylpiperidin-1-yl)methyl)imidazo[1,2-a]pyridin-2-yl)methyl)-5-(pyridin-4-ylethynyl)-2,7-naphthyridin-1(2H)-one (Compound No. 124)

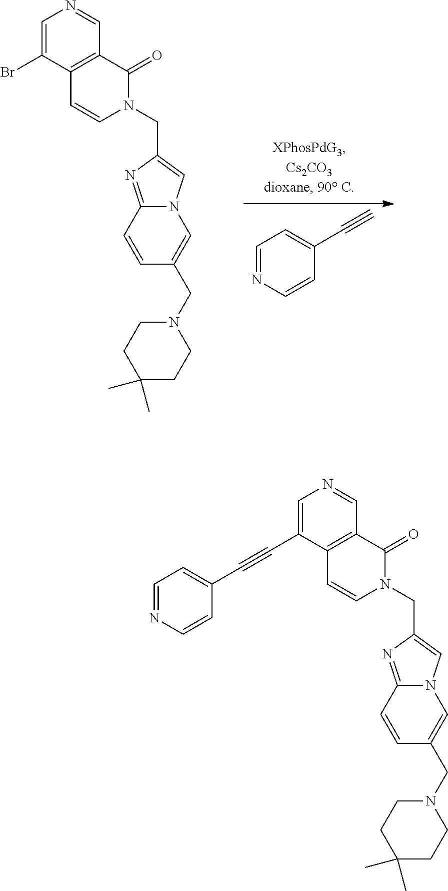

The title compound was prepared using the procedure described for example 111, step 6 with the following modification: the reaction was performed with 3-Ethynylpyridine to give 2-({6-[(4,4-dimethylpiperidin-1-yl)methyl]imidazo[1,2-a]pyridin-2-yl}methyl)-5-[2-(pyridin-4-yl)ethynyl]-1,2-dihydro-2,7-naphthyridin-1-one (yield: 27%) as an off-white solid. ¹H NMR (400 MHz, METHANOL-d4) δ=9.43 (s, 1H), 8.92 (s, 1H), 8.64-8.61 (m, 2H), 8.34 (s, 1H), 7.93 (d, J=7.5 Hz, 1H), 7.86 (s, 1H), 7.67-7.64 (m, 2H), 7.49-7.43 (m, 1H), 7.39-7.34 (m, 1H), 7.03 (d, J=8.1 Hz, 1H), 5.39 (s, 2H), 3.55 (s, 2H), 2.54-2.42((m, 4H), 1.42 (t, J=5.6 Hz, 4H), 0.93 (s, 6H); MS: m/z 502.90 [M+H]⁺

### Synthetic Example 125: Synthesis of 5-{3-[(dimethylamino)methyl]azetidin-1-yl}-2-({6-[(4,4-dimethylpiperidin-1-yl)methyl]imidazo[1,2-a]pyridin-2-yl}methyl)-1,2-dihydro-2,7-naphthyridin-1-one (Compound No. 125)

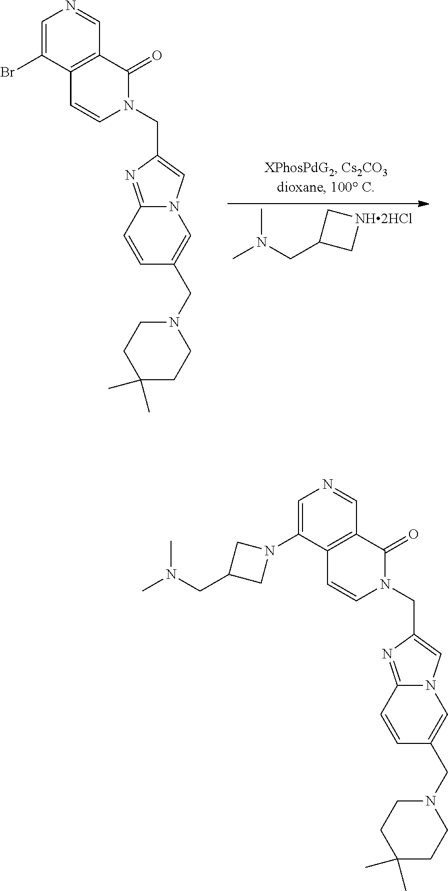

The title compound was prepared using the procedure described for example 111, step 6 with the following modification: the reaction was performed with [(azetidin-3-yl)methyl]dimethylamine to give 5-{3-[(dimethylamino)methyl]azetidin-1-yl}-2-({6-[(4,4-dimethylpiperidin-1-yl)methyl]imidazo[1,2-a]pyridin-2-yl}methyl)-1,2-dihydro-2,7-naphthyridin-1-one (yield: 3%) as off-white solid. ¹H NMR (400 MHz, DMSO-d6) δ=8.78 (s, 1H), 8.37 (s, 1H), 7.86 (s, 1H), 7.82 (s, 1H), 7.67 (d, J=7.8 Hz, 1H), 7.42 (d, J=9.4 Hz, 1H), 7.19-7.13 (m, 1H), 6.60 (d, J=7.6 Hz, 1H), 5.24 (s, 2H), 4.24 (t, J=7.7 Hz, 2H), 3.80-3.73 (m, 2H), 3.41 (s, 2H), 2.34-2.31 (m, 5H), 2.14 (s, 6H), 1.34-1.26 (m, 6H), 0.87-0.83 (m, 6H); MS: m/z 513.90 [M+H]⁺

### Synthetic Example 126: Synthesis of 2-({6-[(4,4-dimethylpiperidin-1-yl)methyl]imidazo[1,2-a]pyridin-2-yl}methyl)-5-[3-(4-methylpiperazin-1-yl)azetidin-1-yl]-1,2-dihydro-2,7-naphthyridin-1-one (Compound No. 126)

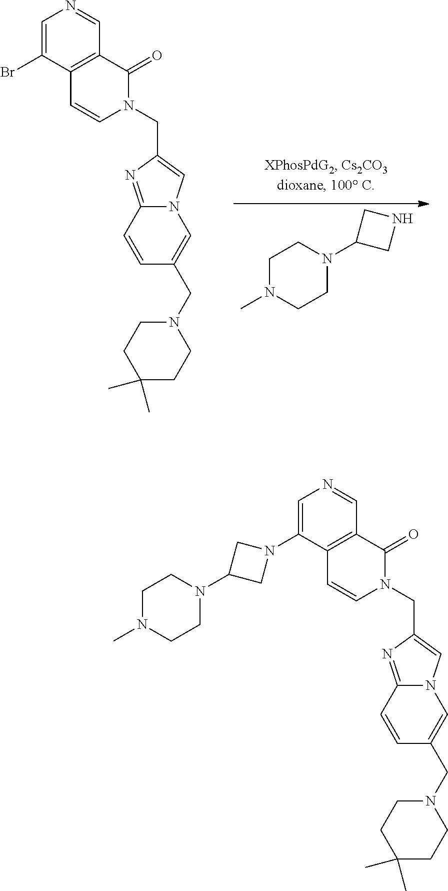 [SMILES: CC1(C)CCN(Cc2ccc3nc(Cn4ccc5c(Br)cncc5c4=O)cn3c2)CC1 ; CN1CCN(C2CNC2)CC1 ; CN1CCN(C2CN(c3cncc4c(=O)n(Cc5cn6cc(CN7CCC(C)(C)CC7)ccc6n5)ccc34)C2)CC1]

The title compound was prepared using the procedure described for example 111, step 6 with the following modification: the reaction was performed with 1-(azetidin-3-yl)-4-methylpiperazine to give 2-({6-[(4,4-dimethylpiperidin-1-yl)methyl]imidazo[1,2-a]pyridin-2-yl}methyl)-5-[3-(4-methylpiperazin-1-yl)azetidin-1-yl]-1,2-dihydro-2,7-naphthyridin-1-one (yield: 8%) as an off white solid. ¹H NMR (400 MHz, DMSO-d6) δ=8.80 (s, 1H), 8.37 (s, 1H), 7.89 (s, 1H), 7.82 (s, 1H), 7.68 (d, J=7.6 Hz, 1H), 7.42 (d, J=9.2 Hz, 1H), 7.19-7.14 (m, 1H), 6.61 (d, J=7.6 Hz, 1H), 5.25 (s, 2H), 4.22 (t, J=7.3 Hz, 2H), 3.92-3.86 (m, 2H), 3.41 (s, 2H), 3.28-3.22 (m, 1H), 2.36-2.31 (m, 8H), 2.15 (s, 3H), 1.34-1.26 (m, 8H), 0.90 (s, 6H); MS: m/z 554.95 [M+H]⁺

### Synthetic Example 127: Synthesis of 2-{[6-({6-azaspiro[2.5]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one (Compound No. 127)

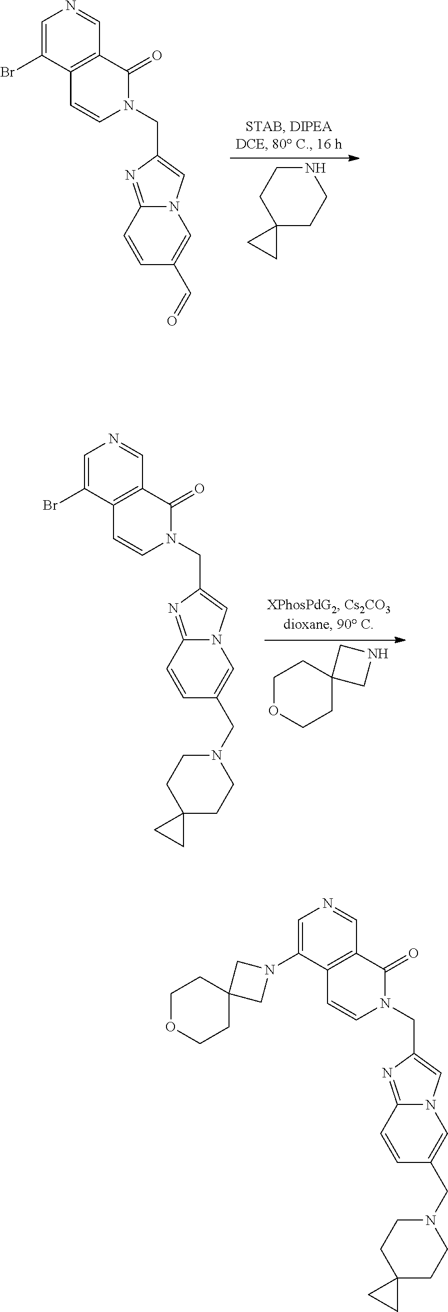

Step 1: To a stirred solution 2-[(5-bromo-1-oxo-1,2-dihydro-2,7-naphthyridin-2-yl)methyl]imidazo[1,2-a]pyridine-6-carbaldehyde (0.1 g, 261 μmol) in 1,2-Dichloroethane (4 mL) was added 6-azaspiro[2.5]octane (112 mg, 783 μmol), followed by the addition of sodium bis(acetyloxy)boranuidyl acetate (332 mg, 1.57 mmol) and DIPEA(202 mg, 1.57 mmol). The reaction mixture was heated at 80° C. for 16 h, cooled to room temperature, diluted with water (10 mL), and extracted with DCM/MeOH 10% (20 mL×2). The combined organic layers were dried over Na₂SO₄, filtered, concentrated in vacuo and purified by flash column chromatography (SiO₂, 2-5% MeOH in DCM) to give 2-{[6-({6-azaspiro[2.5]octan-6-yl}methyl)imidazo[1,2-a]pyridin-2-yl]methyl}-5-bromo-1,2-dihydro-2,7-naphthyridin-1-one (50 mg, yield: 40%) as a white solid. MS: m/z 479.85 [M+H]⁺

Step 2: The title compound was prepared using the procedure described for example 111, step 6 with the following modification: the reaction was performed with 2-{[6-({6-azaspiro[2.5]octan-6-yl}methyl)imidazo[1,2-a]pyridin-2-yl]methyl}-5-bromo-1,2-dihydro-2,7-naphthyridin-1-one and 7-oxa-2-azaspiro[3.5]nonane to give 2-{[6-({6-azaspiro[2.5]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one(yield: 23%) as an off-white solid. ¹H NMR (400 MHz, DMSO-d6) δ=8.78 (s, 1H), 8.39 (s, 1H), 7.87 (s, 1H), 7.82 (s, 1H), 7.69 (d, J=7.8 Hz, 1H), 7.43 (d, J=9.2 Hz, 1H), 7.22-7.17 (m, 1H), 6.63 (d, J=7.8 Hz, 1H), 5.25 (s, 2H), 3.93 (s, 4H), 3.60-3.53 (m, 4H), 3.44 (s, 2H), 2.39 (br s, 4H), 1.80-1.73 (m, 4H), 1.31 (br s, 4H), 0.22 (s, 4H); MS: m/z 525.25 [M+H]⁺

### Synthetic Example 128: Synthesis of 2-{[6-({2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one (Compound No. 128)

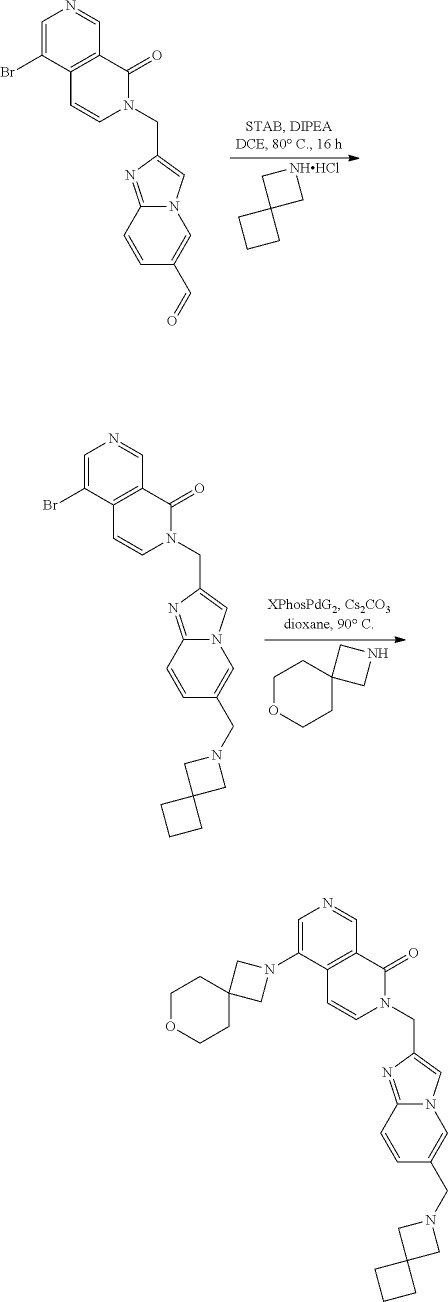

Step 1: The intermediate compound was prepared using the procedure described for example 127, step 1 with the following modification: the reaction was performed with 2-azaspiro[3.3]heptane hydrochloride to give 2-{[6-({2-azaspiro[3.3]heptan-2-yl}methyl)imidazo[1,2-a]pyridin-2-yl]methyl}-5-bromo-1,2-dihydro-2,7-naphthyridin-1-one (25 mg, yield:21%) as a brown solid. [0726]¹H NMR (400 MHz, DMSO-d6) δ=9.29 (s, 1H), 8.94 (s, 1H), 8.34 (s, 1H), 8.04 (d, J=7.6 Hz, 1H), 7.86 (s, 1H), 7.41 (d, J=9.2 Hz, 1H), 7.14-7.07 (m, 1H), 6.71 (d, J=7.5 Hz, 1H), 5.30 (s, 2H), 3.46 (br s, 2H), 3.11 (br s, 4H), 2.03 (br t, J=7.6 Hz, 4H), 1.81-1.69 (m, 2H); MS: m/z 465.90 [M+H]⁺

Step 2: The title compound was prepared using the procedure described for example 127, step 2 with the following modification: the reaction was performed with 2-{[6-({2-azaspiro[3.3]heptan-2-yl}methyl)imidazo[1,2-a]pyridin-2-yl]methyl}-5-bromo-1,2-dihydro-2,7-naphthyridin-1-one to afford 2-{[6-({2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one (8.8 mg yield: 32%) as a yellow solid. ¹H NMR (400 MHz, DMSO-d6) δ=8.78 (s, 1H), 8.34 (s, 1H), 7.88 (s, 1H), 7.81 (s, 1H), 7.68 (d, J=7.6 Hz, 1H), 7.40 (d, J=9.2 Hz, 1H), 7.10 (d, J=10.7 Hz, 1H), 6.63 (d, J=7.8 Hz, 1H), 5.25 (s, 2H), 3.94 (s, 4H), 3.61-3.51 (m, 4H), 3.43 (s, 2H), 3.09 (s, 4H), 2.04-1.99 (m, 4H), 1.82-1.69 (m, 6H); MS: m/z 529.10 [M+H]⁺

### Synthetic Example 129: Synthesis of 2-((6-((7-azaspiro[3.5]nonan-7-yl)methyl)imidazo[1,2-a]pyridin-2-yl)methyl)-5-(7-oxa-2-azaspiro[3.5]nonan-2-yl)-2,7-naphthyridin-1(2H)-one (Compound No. 129)

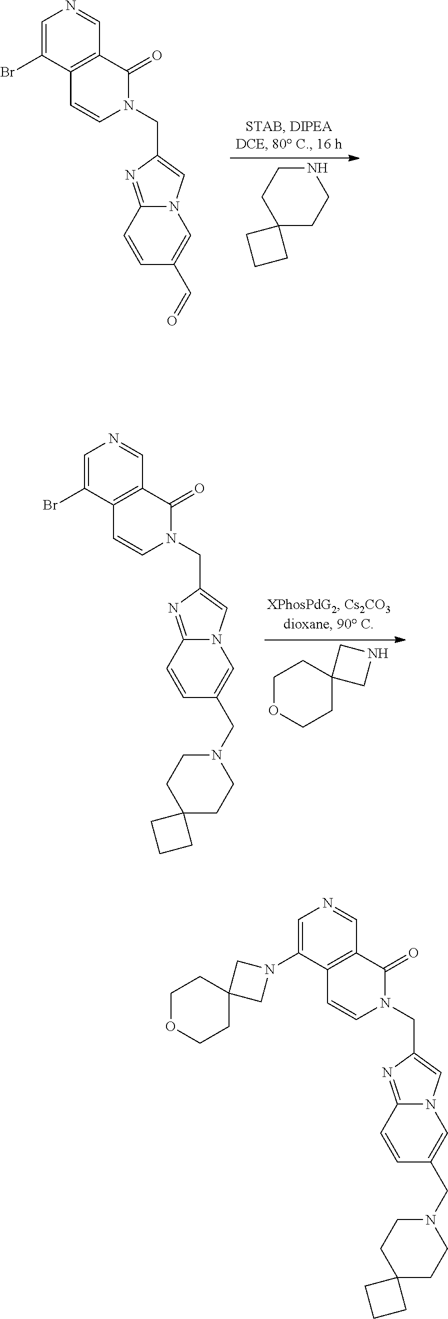

Step 1: The intermediate compound was prepared using the procedure described for example 127, step 1 with the following modification: the reaction was performed with 7-azaspiro[3.5]nonane hydrochloride to give 2-{[6-(({7-azaspiro[3.5]nonan-7-yl}methyl)imidazo[1,2-a]pyridin-2-yl]methyl}-5-bromo-1,2-dihydro-2,7-naphthyridin-1-one (yield: 37%) as a pale yellow semi solid. MS: m/z 534.95 [M+H]⁺

Step 2: The title compound was prepared using the procedure described for example 127, step 2 with the following modification: the reaction was performed with 2-{[6-({7-azaspiro[3.5]nonan-7-yl}methyl)imidazo[1,2-a]pyridin-2-yl]methyl}-5-bromo-1,2-dihydro-2,7-naphthyridin-1-one afford 2-((6-((7-azaspiro[3.5]nonan-7-yl)methyl)imidazo[1,2-a]pyridin-2-yl)methyl)-5-(7-oxa-2-azaspiro[3.5]nonan-2-yl)-2,7-naphthyridin-1(2H)-one (yield: 33%) as a pale brown solid. ¹H NMR (400 MHz, DMSO-d6) δ=8.78 (s, 1H), 8.36 (s, 1H), 7.88 (s, 1H), 7.81 (s, 1H), 7.68 (d, J=7.8 Hz, 1H), 7.42 (d, J=9.2 Hz, 1H), 7.16 (d, J=10.9 Hz, 1H), 6.63 (d, J=7.6 Hz, 1H), 5.25 (s, 2H), 3.93 (s, 4H), 3.60-3.50 (m, 4H), 3.36 (s, 2H), 2.30-2.18 (m, 4H), 1.80-1.74 (m, 4H), 1.70-1.64 (m, 6H), 1.50 (br t, J=5.2 Hz, 4H); MS: m/z 539.10 [M+H]⁺

### Synthetic Example 130: Synthesis of 5-{7-oxa-2-azaspiro[3.5]nonan-2-yl}-2-{[6-({2-oxa-7-azaspiro[4.4]nonan-7-yl}methyl)imidazo[1,2-a]pyridin-2-yl]methyl}-1,2-dihydro-2,7-naphthyridin-1-one (Compound No. 130)

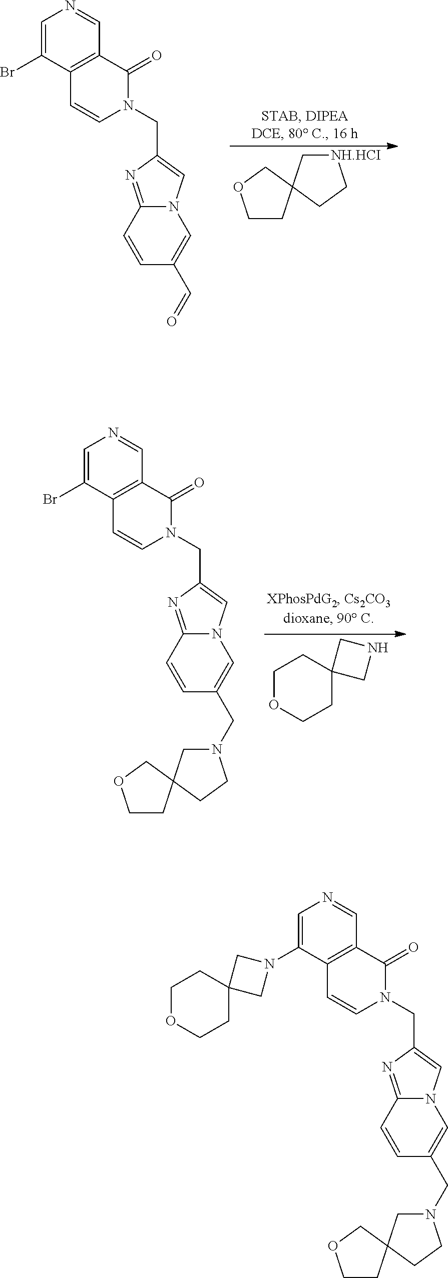

Step 1: The intermediate compound was prepared using the procedure described for example 127, step 1 with the following modification: the reaction was performed with 2-oxa-7-azaspiro[4.4]nonane hydrochloride to give 5-bromo-2-{[6-({2-oxa-7-azaspiro[4.4]nonan-7-yl}methyl)imidazo[1,2-a]pyridin-2-yl]methyl}-1,2-dihydro-2,7-naphthyridin-1-one (yield: 27%) as a yellow solid. MS: m/z 495.75 [M+H]⁺

Step 2: The title compound was prepared using the procedure described for example 127, step 2 with the following modification: the reaction was performed with 5-bromo-2-{[6-({2-oxa-7-azaspiro[4.4]nonan-7-yl}methyl)imidazo[1,2-a]pyridin-2-yl]methyl}-1,2-dihydro-2,7-naphthyridin-1-one to afford 5-{7-oxa-2-azaspiro[3.5]nonan-2-yl}-2-{[6-({2-oxa-7-azaspiro[4.4]nonan-7-yl}methyl)imidazo[1,2-a]pyridin-2-yl]methyl}-1,2-dihydro-2,7-naphthyridin-1-one (yield: 59%) as an off white solid. ¹H NMR (400 MHz, DMSO-d6) δ=8.78 (s, 1H), 8.39 (s, 1H), 7.88 (s, 1H), 7.81 (s, 1H), 7.69 (d, J=7.8 Hz, 1H), 7.43 (d, J=9.2 Hz, 1H), 7.19 (d, J=9.2 Hz, 1H), 6.63 (d, J=7.6 Hz, 1H), 5.25 (s, 2H), 3.93 (s, 4H), 3.73-3.63 (m, 2H), 3.59-3.54 (m, 4H), 3.53 (s, 2H), 3.52-3.48 (m, 2H), 3.41 (d, J=8.1 Hz, 2H), 2.63-2.58 (m, 2H), 1.84-1.73 (m, 8H); MS: m/z 541.20 [M+H]⁺

### Synthetic Example 131: Synthesis of 5-{7-oxa-2-azaspiro[3.5]nonan-2-yl}-2-{[6-({6-oxa-3-azabicyclo[3.1.1]heptan-3-yl}methyl)imidazo[1,2-a]pyridin-2-yl]methyl}-1,2-dihydro-2,7-naphthyridin-1-one (Compound No. 131)

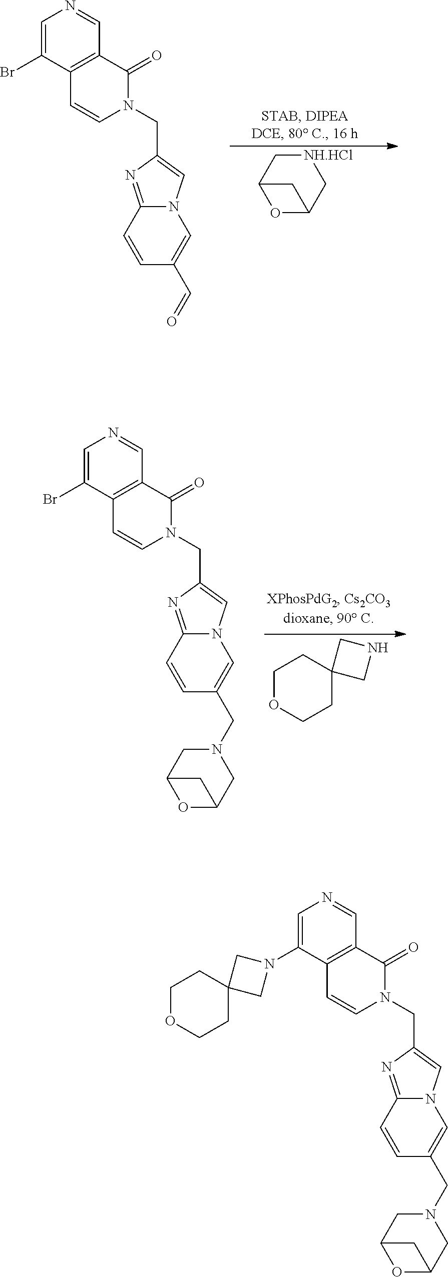

Step 1: The intermediate compound was prepared using the procedure described for example 127, step 1 with the following modification: the reaction was performed with 6-oxa-3-azabicyclo[3.1.1]heptane hydrochloride to give 5-bromo-2-{[6-({6-oxa-3-azabicyclo[3.1.1]heptan-3-yl}methyl)imidazo[1,2-a]pyridin-2-yl]methyl}-1,2-dihydro-2,7-naphthyridin-1-one (yield: 43%) as a pale yellow solid. MS: m/z 467.75 [M+H]⁺

Step 2: The title compound was prepared using the procedure described for example 127, step 2 with the following modification: the reaction was performed with 5-bromo-2-{[6-({6-oxa-3-azabicyclo[3.1.1]heptan-3-yl}methyl)imidazo[1,2-a]pyridin-2-yl]methyl}-1,2-dihydro-2,7-naphthyridin-1-one to give 5-{7-oxa-2-azaspiro[3.5]nonan-2-yl}-2-{[6-({6-oxa-3-azabicyclo[3.1.1]heptan-3-yl}methyl)imidazo[1,2-a]pyridin-2-yl]methyl}-1,2-dihydro-2,7-naphthyridin-1-one (yield: 65%) as a pale yellow solid. ¹H NMR (300 MHz, DMSO-d6) δ=8.78 (s, 1H), 8.46 (s, 1H), 7.88 (s, 1H), 7.81 (s, 1H), 7.69 (d, J=7.9 Hz, 1H), 7.45 (d, J=9.2 Hz, 1H), 7.23 (d, J=9.2 Hz, 1H), 6.64 (d, J=7.9 Hz, 1H), 5.26 (s, 2H), 4.40 (d, J=6.3 Hz, 2H), 3.93 (s, 4H), 3.68 (s, 2H), 3.56 (br t, J=4.8 Hz, 4H), 2.96 (d, J=11.2 Hz, 2H), 2.88-2.78 (m, 1H), 2.66 (d, J=11.2 Hz, 2H), 2.27-2.22 (m, 1H), 1.77(br t, J=4.9 Hz, 4H); MS: m/z 513.15 [M+H]⁺

### Synthetic Example 132: Synthesis of 5-{7-oxa-2-azaspiro[3.5]nonan-2-yl}-2-[(6-{[4-(trifluoromethyl)piperidin-1-yl]methyl}imidazo[1,2-a]pyridin-2-yl)methyl]-1,2-dihydro-2,7-naphthyridin-1-one (Compound No. 132)

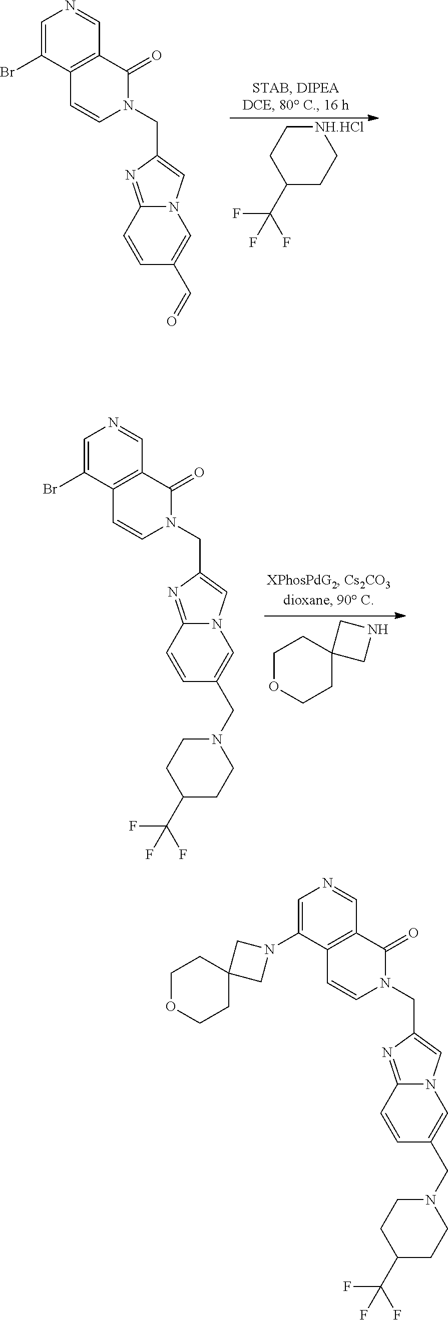

Step 1: The intermediate compound was prepared using the procedure described for example 127, step 1 with the following modification: the reaction was performed with 4-(trifluoromethyl)piperidine to give 5-bromo-2-[(6-{[4-(trifluoromethyl)piperidin-1-yl]methyl}imidazo[1,2-a]pyridin-2-yl)methyl]-1,2-dihydro-2,7-naphthyridin-1-one (yield: 26%) as an off white solid. MS: m/z 520.80 [M+H]⁺

Step 2: The title compound was prepared using the procedure described for example 127, step 2 with the following modification: the reaction was performed with 5-bromo-2-[(6-{[4-(trifluoromethyl)piperidin-1-yl]methyl}imidazo[1,2-a]pyridin-2-yl)methyl]-1,2-dihydro-2,7-naphthyridin-1-one to afford 5-{7-oxa-2-azaspiro[3.5]nonan-2-yl}-2-[(6-{[4-(trifluoromethyl)piperidin-1-yl]methyl}imidazo[1,2-a]pyridin-2-yl)methyl]-1,2-dihydro-2,7-naphthyridin-1-one (yield: 12%) as an off-white solid. ¹H NMR (400 MHz, DMSO-d6) δ=8.78 (s, 1H), 8.40 (s, 1H), 7.88 (s, 1H), 7.81 (s, 1H), 7.69 (d, J=7.6 Hz, 1H), 7.44 (d, J=9.2 Hz, 1H), 7.17 (d, J=9.4 Hz, 1H), 6.64 (d, J=7.6 Hz, 1H), 5.26 (s, 2H), 3.94 (s, 4H), 3.61-3.50 (m, 4H), 3.44 (s, 2H), 2.88 (br d, J=11.3 Hz, 2H), 2.30-2.21 (m, 1H), 1.97 (br t, J=11.1 Hz, 2H), 1.82-1.70 (m, 6H), 1.51-1.37 (m, 2H); MS: m/z 567.00 [M+H]⁺

### Synthetic Example 133: Synthesis of 2-{[6-({8-methyl-3,8-diazabicyclo[3.2.1]octan-3-yl}methyl)imidazo[1,2-a]pyridin-2-yl]methyl}-5-{7-oxa-2-azaspiro[3.5]nonan-2-yl}-1,2-dihydro-2,7-naphthyridin-1-one (Compound No. 133)

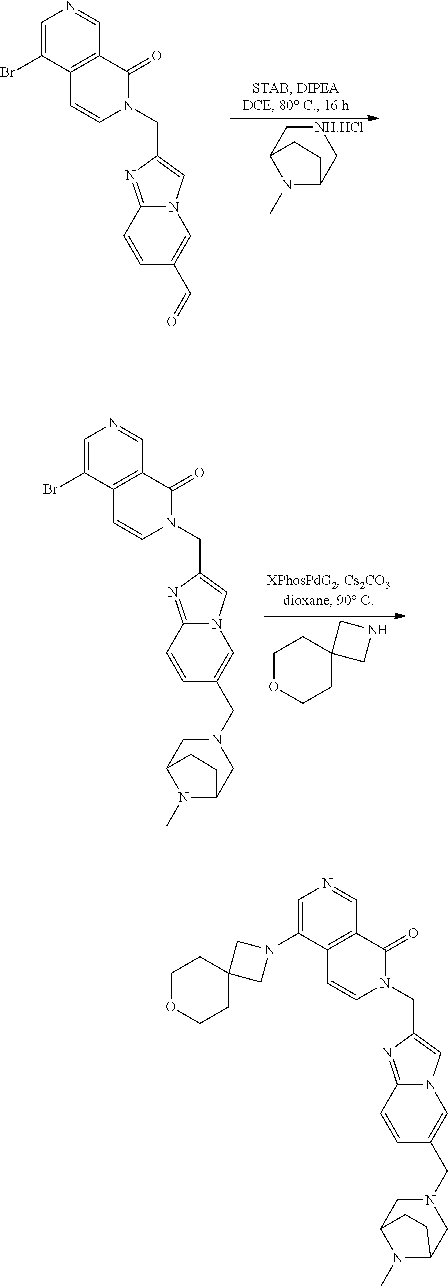

Step 1: The intermediate compound was prepared using the procedure described for example 127, step 1 with the following modification: the reaction was performed with 8-methyl-3,8-diazabicyclo[3.2.1]octane hydrochloride to give 5-bromo-2-{[6-({8-methyl-3,8-diazabicyclo[3.2.1]octan-3-yl}methyl)imidazo[1,2-a]pyridin-2-yl]methyl}-1,2-dihydro-2,7-naphthyridin-1-one (yield: 45%) as a brown solid. MS: m/z 494.80 [M+H]⁺

Step 2: The title compound was prepared using the procedure described for example 127, step 2 with the following modification: the reaction was performed with 5-bromo-2-{[6-({8-methyl-3,8-diazabicyclo[3.2.1]octan-3-yl}methyl)imidazo[1,2-a]pyridin-2-yl]methyl}-1,2-dihydro-2,7-naphthyridin-1-one to afford 2-{[6-({8-methyl-3,8-diazabicyclo[3.2.1]octan-3-yl}methyl)imidazo[1,2-a]pyridin-2-yl]methyl}-5-(7-oxa-2-azaspiro[3.5]nonan-2-yl)-1,2-dihydro-2,7-naphthyridin-1-one (yield: 18%) as an off-white solid. ¹H NMR (400 MHz, DMSO-d6) δ=8.78 (s, 1H), 8.40 (s, 1H), 7.88 (s, 1H), 7.81 (s, 1H), 7.68 (d, J=7.8 Hz, 1H), 7.43 (d, J=9.2 Hz, 1H), 7.26-7.18 (m, 1H), 6.63 (d, J=7.8 Hz, 1H), 5.25 (s, 2H), 3.94 (s, 4H), 3.60-3.52 (m, 4H), 3.41 (s, 3H), 3.07-3.01 (m, 2H), 2.45-2.42 (m, 2H), 2.13 (br d, J=9.8 Hz, 2H), 2.04-1.95 (m, 2H), 1.90-1.82 (m, 2H), 1.80-1.75 (m, 4H), 1.68 (d, J=6.7 Hz, 2H); MS: m/z 540.00 [M+H]⁺

### Synthetic Example 134: Synthesis of 4-methyl-1-({2-[(5-{7-oxa-2-azaspiro[3.5]nonan-2-yl}-1-oxo-1,2-dihydro-2,7-naphthyridin-2-yl)methyl]imidazo[1,2-a]pyridin-6-yl}methyl)piperidine-4-carbonitrile (Compound No. 134)

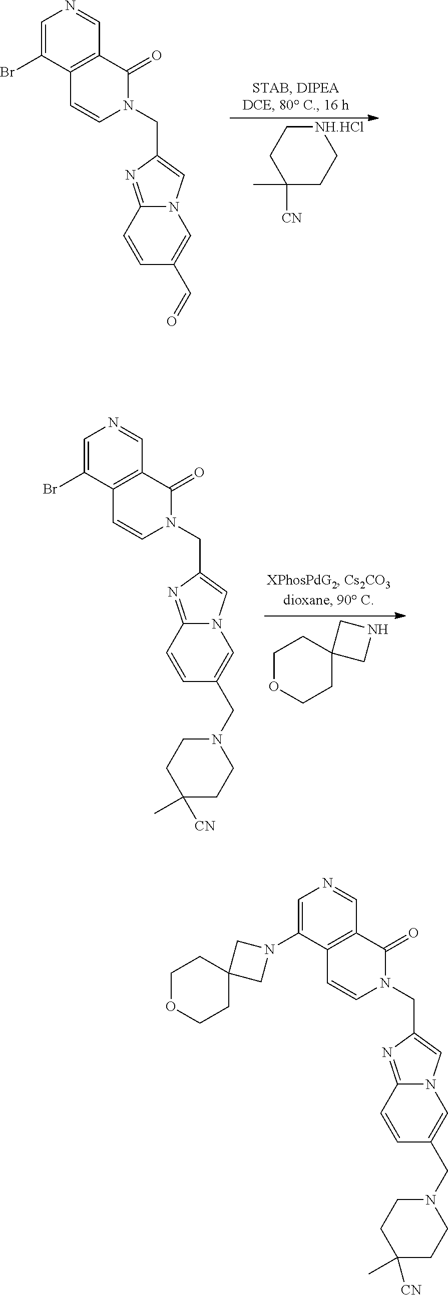

Step 1: The intermediate compound was prepared using the procedure described for example 127, step 1 with the following modification: the reaction was performed with 4-methylpiperidine-4-carbonitrile hydrochloride to give 1-((2-[(5-bromo-1-oxo-1,2-dihydro-2,7-naphthyridin-2-yl)methyl]imidazo[1,2-a]pyridin-6-yl)methyl)-4-methylpiperidine-4-carbonitrile (yield: 23%). MS: m/z 492.45 [M+H]⁺

Step 2: The title compound was prepared using the procedure described for example 127, step 2 with the following modification: the reaction was performed with 1-((2-[(5-bromo-1-oxo-1,2-dihydro-2,7-naphthyridin-2-yl)methyl]imidazo[1,2-a]pyridin-6-yl)methyl)-4-methylpiperidine-4-carbonitrile to give 4-methyl-1-({2-[(5-{7-oxa-2-azaspiro[3.5]nonan-2-yl}-1-oxo-1,2-dihydro-2,7-naphthyridin-2-yl)methyl]imidazo[1,2-a]pyridin-6-yl}methyl)piperidine-4-carbonitrile (yield: 32%) as an off white solid. ¹H NMR (400 MHz, DMSO-d6) δ=8.78 (s, 1H), 8.40 (s, 1H), 7.88 (s, 1H), 7.81 (s, 1H), 7.68 (d, J=7.6 Hz, 1H), 7.44 (d, J=9.2 Hz, 1H), 7.17 (d, J=9.2 Hz, 1H), 6.63 (d, J=7.8 Hz, 1H), 5.26 (s, 2H), 3.93 (s, 4H), 3.61-3.52 (m, 4H), 3.46 (s, 2H), 2.78 (brd, J=12.3 Hz, 2H), 2.44 (brs, 2H), 2.14(brt, J=11.21 Hz, 2H), 1.87-1.75 (m, 4H), 1.56-1.41 (m, 2H), 1.31 (s, 3H); MS: m/z 538.85 [M+H]⁺

### Synthetic Example 135: Synthesis of 2-{[6-({6,6-difluoro-2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one (Compound No. 135)

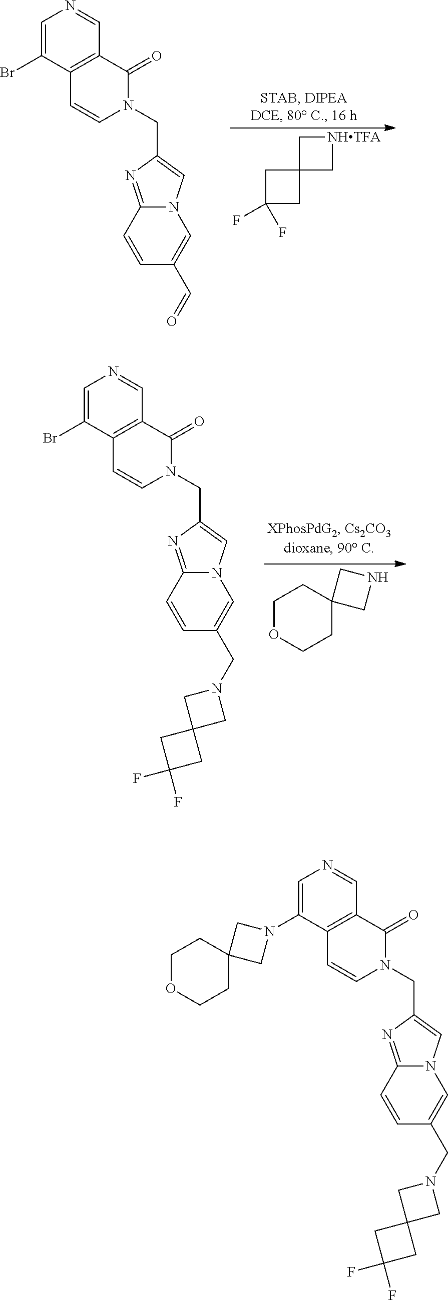

Step 1: The intermediate compound was prepared using the procedure described for example 127, step 1 with the following modification: the reaction was performed with 6,6-difluoro-2-azaspiro[3.3]heptane to give 5-bromo-2-{[6-({6,6-difluoro-2-azaspiro[3.3]heptan-2-yl}methyl)-4λ⁴-imidazo[1,2-a]pyridin-2-yl]methyl}-1,2-dihydro-2,7-naphthyridin-1-one (yield: 88%) as a pale yellow semi solid. MS: m/z 500.05 [M+H]⁺

Step 2: The title compound was prepared using the procedure described for example 127, step 2 with the following modification: the reaction was performed with 5-bromo-2-{[6-({6,6-difluoro-2-azaspiro[3.3]heptan-2-yl}methyl)imidazo[1,2-a]pyridin-2-yl]methyl}-1,2-dihydro-2,7-naphthyridin-1-one to afford 2-{[6-({6,6-difluoro-2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one (yield: 26%) as a pale yellow solid. ¹H NMR (400 MHz, DMSO-d6) δ=8.78 (s, 1H), 8.36 (br s, 1H), 7.88 (s, 1H), 7.81 (s, 1H), 7.68 (d, J=7.8 Hz, 1H), 7.42 (br d, J=9.1 Hz, 1H), 7.12 (br d, J=9.7 Hz, 1H), 6.63 (d, J=7.6 Hz, 1H), 5.25 (s, 2H), 3.94 (s, 4H), 3.59-3.53 (m, 4H), 3.52-3.45 (m, 2H), 3.22 (br s, 4H), 2.74-2.68 (m, 4H), 1.77 (br s, 4H); MS: m/z 547.15 [M+H]⁺

### Synthetic Example 136: Synthesis of 2-{[6-({6-fluoro-2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one (Compound No. 136)

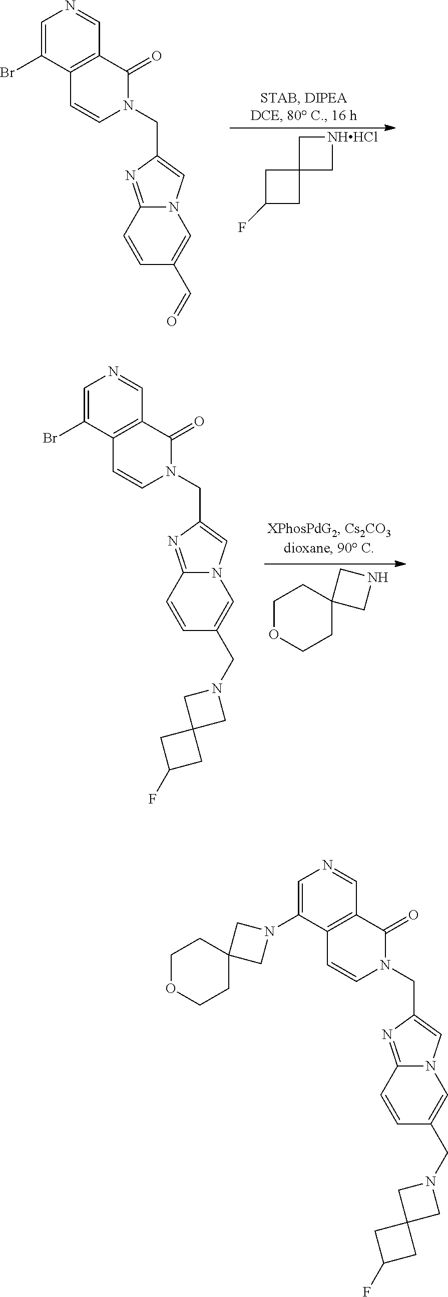

Step 1: The intermediate compound was prepared using the procedure described for example 127, step 1 with the following modification: the reaction was performed with 6-fluoro-2-azaspiro[3.3]heptane to give 5-bromo-2-([6-((6-fluoro-2-azaspiro[3.3]heptan-2-yl)methyl)imidazo[1,2-a]pyridin-2-yl]methyl)-1,2-dihydro-2,7-naphthyridin-1-one (yield: 79%) as a brown sticky solid MS: m/z 482.85 [M+H]⁺

Step 2: The title compound was prepared using the procedure described for example 127, step 2 with the following modification: the reaction was performed with 5-bromo-2-{[6-({6-fluoro-2-azaspiro[3.3]heptan-2-yl}methyl)imidazo[1,2-a]pyridin-2-yl]methyl}-1,2-dihydro-2,7-naphthyridin-1-one to give 2-{[6-({6-fluoro-2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one (yield: 27%) as a pale yellow solid. ¹H NMR (400 MHz, DMSO-d6) δ=10.37 (br s, 1H), 8.79 (s, 1H), 8.69 (s, 1H), 8.10 (s, 1H), 7.94 (d, J=7.8 Hz, 1H), 7.85 (s, 1H), 7.67 (d, J=9.2 Hz, 1H), 7.42-7.36 (m, 1H), 6.80 (d, J=7.8 Hz, 1H), 5.38 (s, 2H), 5.08-4.83 (m, 1H), 4.36 (br s, 2H), 4.24-4.09 (m, 2H), 4.03 (s, 4H), 4.02-3.96 (m, 2H), 3.62-3.52 (m, 4H), 2.65-2.56 (m, 2H), 2.46-2.34 (m, 2H), 1.84-1.74 (m, 4H); MS: m/z 528.80 [M+H]⁺

### Synthetic Example 137: Synthesis of 2-{[6-({2-azaspiro[4.4]nonan-2-yl}methyl)imidazo[1,2-a]pyridin-2-yl]methyl)}-5-{7-oxa-2-azaspiro[3.5]nonan-2-yl}-1,2-dihydro-2,7-naphthyridin-1-one (Compound No. 137)

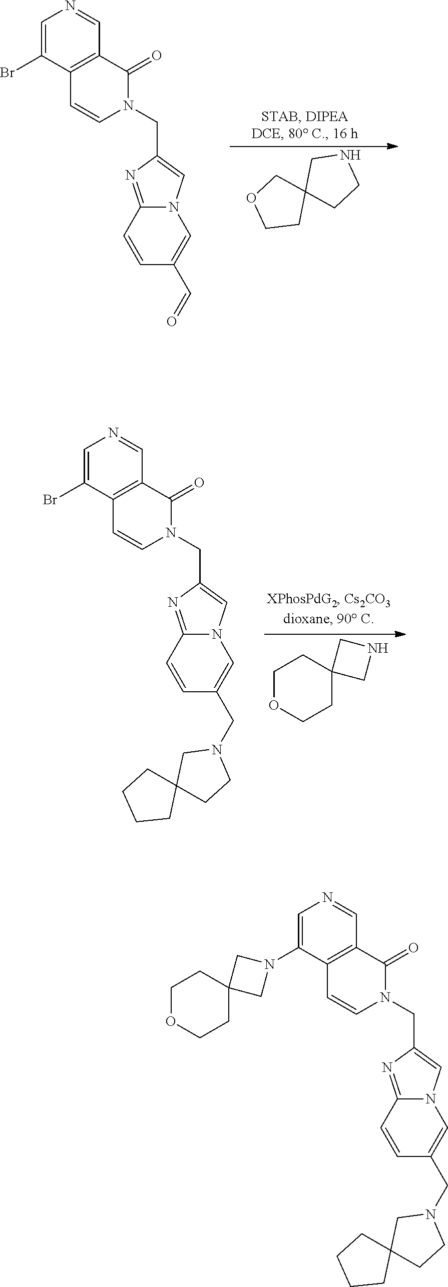

Step 1: The intermediate compound was prepared using the procedure described for example 127, step 1 with the following modification: the reaction was performed with 2-azaspiro[4.4]nonane to give 2-{[6-({2-azaspiro[4.4]nonan-2-yl}methyl)imidazo[1,2-a]pyridin-2-yl]methyl}-5-bromo-1,2-dihydro-2,7-naphthyridin-1-one (yield: 89%). MS: m/z 492.10 [M+H]⁺

Step 2: The title compound was prepared using the procedure described for example 127, step 2 with the following modification: the reaction was performed with 2-{[6-({2-azaspiro[4.4]nonan-2-yl}methyl)imidazo[1,2-a]pyridin-2-yl]methyl}-5-bromo-1,2-dihydro-2,7-naphthyridin-1-one to give 2-{[6-({2-azaspiro[4.4]nonan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one (yield: 26%) as an off-white solid. ¹H NMR (400 MHz, DMSO-d6) δ=8.78 (s, 1H), 8.38 (s, 1H), 7.88 (s, 1H), 7.81 (s, 1H), 7.69 (d, J=7.8 Hz, 1H), 7.43 (d, J=9.1 Hz, 1H), 7.19 (br d, J=9.2 Hz, 1H), 6.64 (br d, J=7.8 Hz, 1H), 5.25 (s, 2H), 3.94 (s, 4H), 3.61-3.54((m, 4H), 3.51 (s, 2H), 2.33 (br s, 2H), 1.83-1.74 (m, 4H), 1.63 (br t, J=7.0 Hz, 2H), 1.56-1.45 (m, 10H); MS: m/z 539.15 [M+H]⁺

### Synthetic Example 138: Synthesis of 2-({6-[(4-methylpiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one (Compound No. 138)

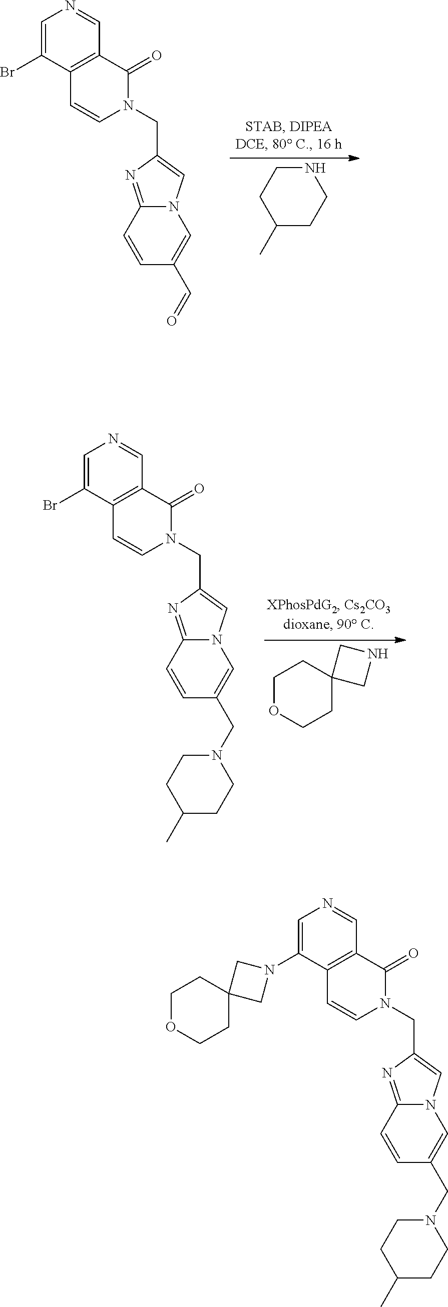

Step 1: The intermediate compound was prepared using the procedure described for example 127, step 1 with the following modification: the reaction was performed with 4-methylpiperidine to afford 5-bromo-2-({6-[(4-methylpiperidin-1-yl)methyl]imidazo[1,2-a]pyridin-2-yl}methyl)-1,2-dihydro-2,7-naphthyridin-1-one (yield: 76%) as a brown solid. MS: m/z 467.45 [M+H]⁺

Step 2: The title compound was prepared using the procedure described for example 127, step 2 with the following modification: the reaction was performed with 5-bromo-2-({6-[(4-methylpiperidin-1-yl)methyl]imidazo[1,2-a]pyridin-2-yl}methyl)-1,2-dihydro-2,7-naphthyridin-1-one to give 2-({6-[(4-methylpiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one (yield: 25%) as an off white solid. ¹H NMR (400 MHz, DMSO-d6) δ=8.78 (s, 1H), 8.37 (s, 1H), 7.88 (s, 1H), 7.81 (s, 1H), 7.68 (d, J=7.8 Hz, 1H), 7.42 (d, J=9.2 Hz, 1H), 7.20-7.13 (m, 1H), 6.63 (d, J=8.1 Hz, 1H), 5.25 (s, 2H), 3.93 (s, 4H), 3.56(brt, J=5.1 Hz, 4H), 3.39 (s, 2H), 2.75 (br d, J=11.3 Hz, 2H), 2.06-1.96 (m, 1H), 1.94-1.85 (m, 2H), 1.80-1.73 (m, 4H), 1.54 (br d, J=14.5 Hz, 2H), 1.18-1.05 (m, 2H), 0.87 (d, J=6.5 Hz, 3H); MS: m/z 513.20 [M+H]⁺

### Synthetic Example 139: Synthesis of 2-({6-[(4,4-difluoropiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one (Compound No. 139)

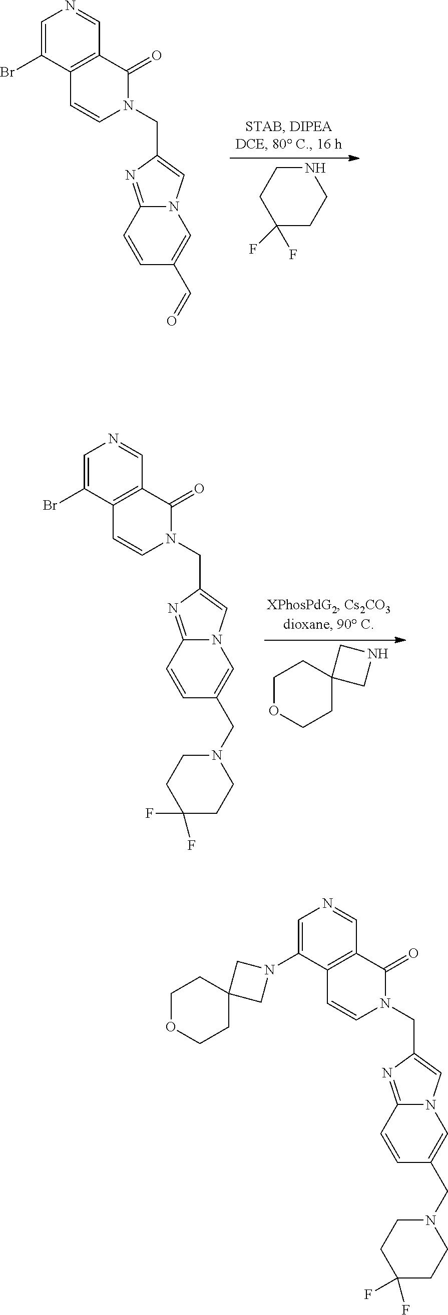

Step 1: The intermediate compound was prepared using the procedure described for example 127, step 1 with the following modification: the reaction was performed with 4,4-difluoropiperidine to give 5-bromo-2-({6-[(4,4-difluoropiperidin-1-yl)methyl]imidazo[1,2-a]pyridin-2-yl}methyl)-1,2-dihydro-2,7-naphthyridin-1-one (yield: 42%) as a pale yellow solid. MS: m/z 487.80 [M+H]⁺

Step 2: The title compound was prepared using the procedure described for ex ample 127, step 2 with the following modification: the reaction was performed with 5-bromo-2-({6-[(4,4-difluoropiperidin-1-yl)methyl]imidazo[1,2-a]pyridin-2-yl}methyl)-1,2-dihydro-2,7-naphthyridin-1-one to give 2-({6-[(4,4-difluoropiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one (yield: 11%) as an off-white solid. ¹H NMR (400 MHz, METHANOL-d4) δ=8.85 (s, 1H), 8.32 (s, 1H), 7.83 (s, 1H), 7.80 (s, 1H), 7.63 (d, J=7.8 Hz, 1H), 7.50-7.43 (m, 1H), 7.39-7.32 (m, 1H), 6.77 (d, J=7.8 Hz, 1H), 5.33 (m, 2H), 3.99 (s, 4H), 3.67 (t, J=5.2 Hz, 4H), 3.56 (s, 2H), 2.57 (br t, J=5.2 Hz, 4H), 2.06-1.91 (m, 4H), 1.87 (t, J=5.2 Hz, 1H); MS: m/z 535.10 [M+H]⁺

### Synthetic Example 140: Synthesis of 2-{[6-({2,2-difluoro-6-azaspiro[3.4]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one (Compound No. 140)

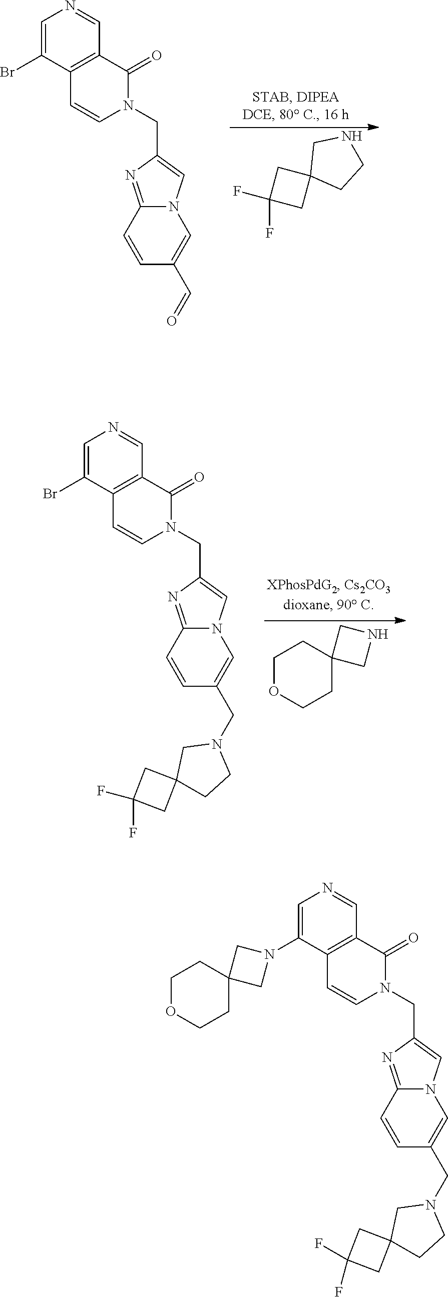

Step 1: The intermediate compound was prepared using the procedure described for example 127, step 1 with the following modification: the reaction was performed with 2,2-difluoro-6-azaspiro[3.4]octane to give 5-bromo-2-([6-((2,2-difluoro-6-azaspiro[3.4]octan-6-yl)methyl)imidazo[1,2-a]pyridin-2-yl]methyl)-1,2-dihydro-2,7-naphthyridin-1-one (yield: 33%) as a brown solid. MS: m/z 513.75 [M+H]⁺

Step 2: The title compound was prepared using the procedure described for example 127, step 2 with the following modification: the reaction was performed with 5-bromo-2-([6-((2,2-difluoro-6-azaspiro[3.4]octan-6-yl)methyl)imidazo[1,2-a]pyridin-2-yl]methyl)-1,2-dihydro-2,7-naphthyridin-1-one (36 mg, 70 μmol) to afford 2-([6-((2,2-difluoro-6-azaspiro[3.4]octan-6-yl)methyl)imidazo[1,2-a]pyridin-2-yl]methyl)-5-(7-oxa-2-azaspiro[3.5]nonan-2-yl)-1,2-dihydro-2,7-naphthyridin-1-one (yield: 69%) as a yellow sticky solid. ¹H NMR (400 MHz, DMSO-d6) δ=10.18 (br s, 1H), 8.79 (s, 1H), 8.71 (s, 1H), 8.09 (s, 1H), 7.93 (d, J=7.6 Hz, 1H), 7.85 (s, 1H), 7.68 (d, J=9.2 Hz, 1H), 7.46 (d, J=10.4 Hz, 1H), 6.79 (d, J=7.8 Hz, 1H), 5.37 (s, 2H), 4.41 (br d, J=10.6 Hz, 2H), 4.03 (s, 4H), 3.62-3.51 (m, 4H), 3.54-3.42 (m, 2H), 3.37-3.21 (m, 2H), 2.85-2.59 (m, 4H), 2.29-2.19 (m, 1H), 2.13-2.02 (m, 1H), 1.79(br t, J=5.1 Hz, 4H); MS: m/z 561.1 [M+H]⁺

### Synthetic Example 141: Synthesis of 2-{[6-({1,1-difluoro-6-azaspiro[2.5]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one (Compound No. 141)

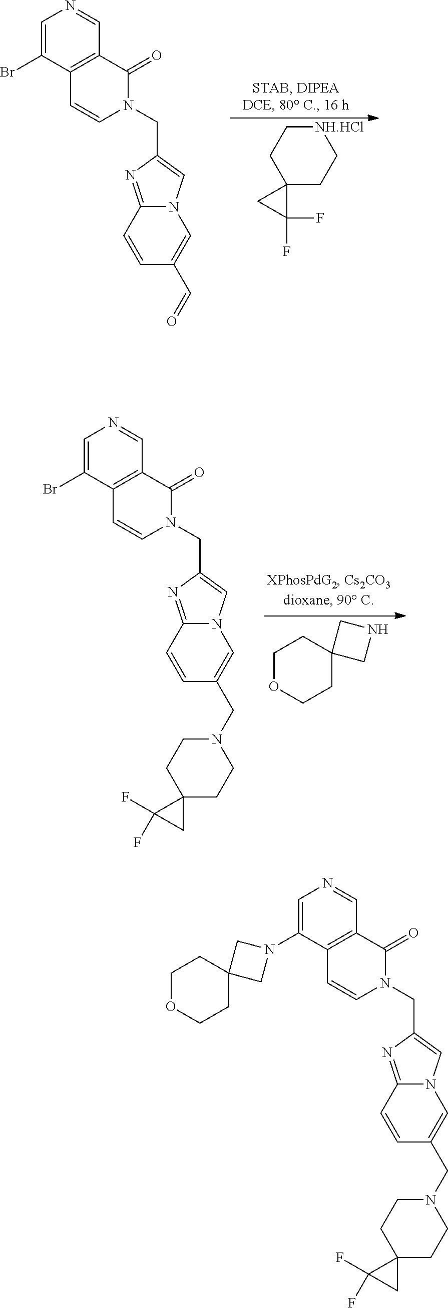

Step 1: The intermediate compound was prepared using the procedure described for example 127, step 1 with the following modification: the reaction was performed with 1,1-difluoro-6-azaspiro[2.5]octane to give 5-bromo-2-([6-((1,1-difluoro-6-azaspiro[2.5]octan-6-yl)methyl)imidazo[1,2-a]pyridin-2-yl]methyl)-1,2-dihydro-2,7-naphthyridin-1-one (yield: 47%) as a brown solid. MS: m/z 514.1 [M+H]⁺

Step 2: The title compound was prepared using the procedure described for example 127, step 2 with the following modification: the reaction was performed with 5-bromo-2-([6-((1,1-difluoro-6-azaspiro[2.5]octan-6-yl)methyl)imidazo[1,2-a]pyridin-2-yl]methyl)-1,2-dihydro-2,7-naphthyridin-1-one to give 2-{[6-((1,1-difluoro-6-azaspiro[2.5]octan-6-yl)methyl)imidazo[1,2-a]pyridin-2-yl]methyl}-5-(7-oxa-2-azaspiro[3.5]nonan-2-yl)-1,2-dihydro-2,7-naphthyridin-1-one (yield: 31%) as a pale yellow solid. ¹H NMR (400 MHz, DMSO-d6) δ=8.78 (s, 1H), 8.40 (s, 1H), 7.88 (s, 1H), 7.82 (s, 1H), 7.69 (d, J=7.6 Hz, 1H), 7.44 (d, J=9.2 Hz, 1H), 7.23-7.15 (m, 1H), 6.64 (d, J=7.6 Hz, 1H), 5.26 (s, 2H), 3.94 (s, 4H), 3.61-3.53 (m, 4H), 3.46 (s, 2H), 2.42-2.38 (m, 4H), 1.80-1.74 (m, 4H), 1.59-1.49 (m, 4H), 1.24 (br s, 2H); MS: m/z 561.00 [M+H]⁺

### Synthetic Example 142: Synthesis of 5-{6,6-difluoro-2-azaspiro[3.3]heptan-2-yl}-2-{[6-({[(3-fluorocyclobutyl)methyl]amino}methyl)imidazo[1,2-a]pyridin-2-yl]methyl}-1,2-dihydro-2,7-naphthyridin-1-one. (Compound 142)

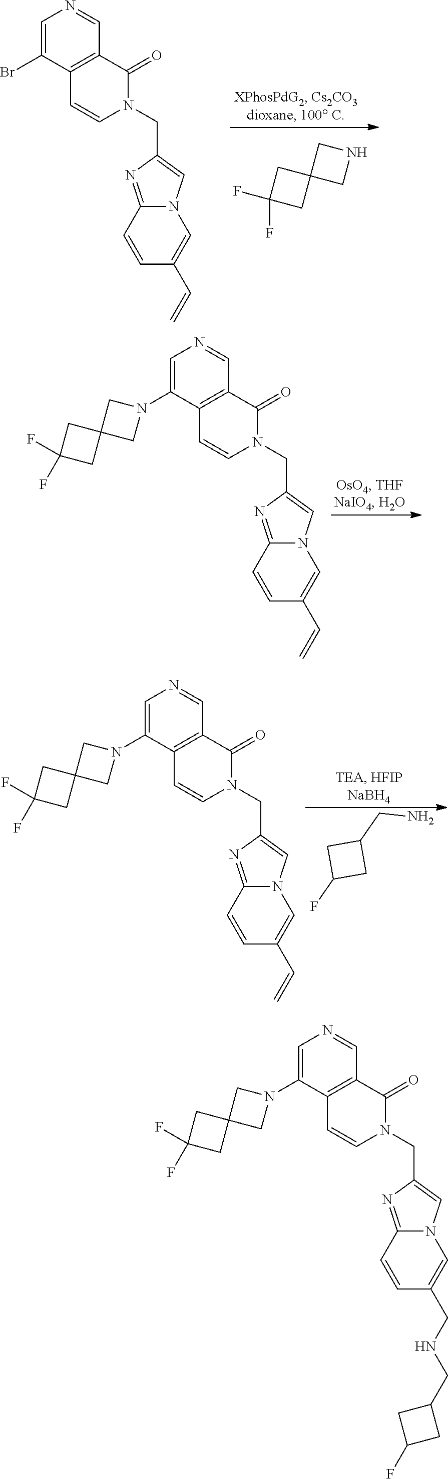

Step 1: The intermediate compound was prepared using the procedure described for example 111, step 6 with the following modification: the reaction was performed with 5-bromo-2-({6-ethenylimidazo[1,2-a]pyridin-2-yl}methyl)-1,2-dihydro-2,7-naphthyridin-1-one and 6,6-difluoro-2-azaspiro[3.3]heptane to give 5-{6,6-difluoro-2-azaspiro[3.3]heptan-2-yl}-2-({6-ethenylimidazo[1,2-a]pyridin-2-yl}methyl)-1,2-dihydro-2,7-naphthyridin-1-one (yield: 79%) as a pale yellow solid. ¹H NMR (300 MHz, DMSO-d6) δ=8.82 (s, 1H), 8.53 (s, 1H), 7.90 (s, 1H), 7.83 (s, 1H), 7.72 (d, J=7.9 Hz, 1H), 7.57-7.43 (m, 2H), 6.76-6.62 (m, 1H), 6.57 (d, J=7.6 Hz, 1H), 5.83 (d, J=17.5 Hz, 1H), 5.30 (d, J=10.9 Hz, 1H), 5.26 (s, 2H), 4.23 (s, 4H), 2.88 (t, J=12.5 Hz, 4H); MS: m/z 434.05 [M+H]⁺

Step 2: The intermediate compound was prepared using the procedure described for example 111, step 4 with the following modification: the reaction was performed with 5-{6,6-difluoro-2-azaspiro[3.3]heptan-2-yl}-2-({6-ethenylimidazo[1,2-a]pyridin-2-yl}methyl)-1,2-dihydro-2,7-naphthyridin-1-one to give 2-[(5-{6,6-difluoro-2-azaspiro[3.3]heptan-2-yl}-1-oxo-1,2-dihydro-2,7-naphthyridin-2-yl)methyl]imidazo[1,2-a]pyridine-6-carbaldehyde (yield: 43%) as an off-white solid. MS: m/z 436.05 [M+H]⁺

Step 3: The title compound was prepared using the procedure described for example 72, step 2 with the following modification: the reaction was performed with 2-[(5-{6,6-difluoro-2-azaspiro[3.3]heptan-2-yl}-1-oxo-1,2-dihydro-2,7-naphthyridin-2-yl)methyl]imidazo[1,2-a]pyridine-6-carbaldehyde and 1-(3-fluorocyclobutyl)methanamine hydrochloride to give 5-{6,6-difluoro-2-azaspiro[3.3]heptan-2-yl}-2-([6-({[(3-fluorocyclobutyl)methyl]amino)methyl)imidazo[1,2-a]pyridin-2-yl]methyl}-1,2-dihydro-2,7-naphthyridin-1-one (yield: 27%) as a pale yellow solid. ¹H NMR (400 MHz, DMSO-d6) δ=8.82 (s, 1H), 8.36 (s, 1H), 7.90 (s, 1H), 7.82 (s, 1H), 7.71 (d, J=7.8 Hz, 1H), 7.43 (d, J=9.2 Hz, 1H), 7.22 (d, J=9.4 Hz, 1H), 6.57 (d, J=7.8 Hz, 1H), 5.25 (s, 2H), 5.18-4.97 (m, 1H), 4.23 (s, 4H), 3.64 (s, 2H), 2.88 (t, J=12.5 Hz, 4H), 2.46 (br s, 2H), 2.27-1.98 (m, 5H); MS: m/z 523.05 [M+H]⁺

### Synthetic Example 143: Synthesis of 2-{[6-({[(3-fluorocyclobutyl)methyl]amino}methyl)imidazo[1,2-a]pyridin-2-yl]methyl}-5-{2-oxa-6-azaspiro[3.3]heptan-6-yl}-1,2-dihydro-2,7-naphthyridin-1-one. (Compound No. 143)

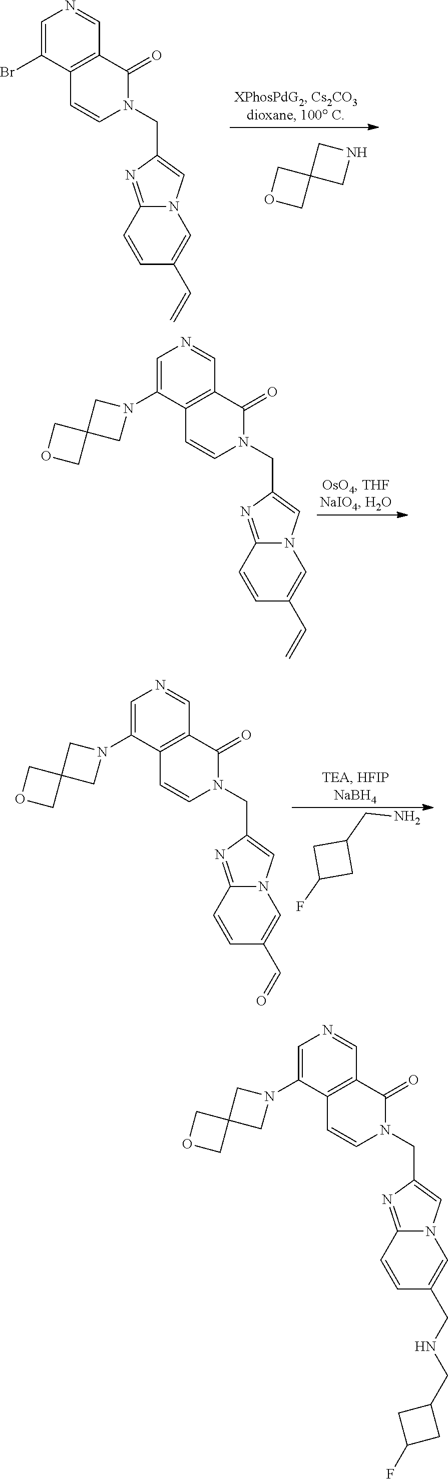

Step 1: The intermediate compound was prepared using the procedure described for example 142, step 1 with the following modification: the reaction was performed with 2-oxa-6-azaspiro[3.3]heptane to give 2-({6-ethenylimidazo[1,2-a]pyridin-2-yl}methyl)-5-{2-oxa-6-azaspiro[3.3]heptan-6-yl}-1,2-dihydro-2,7-naphthyridin-1-one (yield: 8%) as a white solid. MS: m/z 399.65 [M+H]⁺

Step 2: The intermediate compound was prepared using the procedure described for example 142, step 2 with the following modification: the reaction was performed with 2-({6-ethenylimidazo[1,2-a]pyridin-2-yl}methyl)-5-{2-oxa-6-azaspiro[3.3]heptan-6-yl}-1,2-dihydro-2,7-naphthyridin-1-one to give 2-[(5-{2-oxa-6-azaspiro[3.3]heptan-6-yl}-1-oxo-1,2-dihydro-2,7-naphthyridin-2-yl)methyl]imidazo[1,2-a]pyridine-6-carbaldehyde (yield: 31%) as an off-white solid. MS: m/z 402.0 [M+H]⁺

Step 3: The title compound was prepared using the procedure described for example 142, step 3 with the following modification: the reaction was performed with 2-[(5-{2-oxa-6-azaspiro[3.3]heptan-6-yl}-1-oxo-1,2-dihydro-2,7-naphthyridin-2-yl)methyl]imidazo[1,2-a]pyridine-6-carbaldehyde to give 2-{[6-({[(3-fluorocyclobutyl)methyl]amino}methyl)imidazo[1,2-a]pyridin-2-yl]methyl}-5-{2-oxa-6-azaspiro[3.3]heptan-6-yl}-1,2-dihydro-2,7-naphthyridin-1-one (yield: 20%) as an off-white solid. ¹H NMR (400 MHz, METHANOL-d4) δ=8.90-8.86 (m, 1H), 8.39 (s, 1H), 7.83 (s, 1H), 7.84 (s, 1H), 7.67 (d, J=7.8 Hz, 1H), 7.50 (d, J=9.4 Hz, 1H), 7.36 (d, J=9.4 Hz, 1H), 6.73 (d, J=7.8 Hz, 1H), 5.33 (s, 2H), 5.16-4.93 (m, 1H), 4.34 (s, 4H), 3.90 (s, 2H), 2.79 (d, J=7.8 Hz, 2H), 2.62-2.47 (m, 1H), 2.42-2.25 (m, 2H), 2.23-2.08 (m, 2H), 1.95 (s, 4H); MS: m/z 489.15 [M+H]⁺

### Synthetic Example 144: Synthesis of 2-{[6-({[(3-fluorocyclobutyl)methyl]amino}methyl)imidazo[1,2-a]pyridin-2-yl]methyl}-5-{7-oxa-2-azaspiro[3.5]nonan-2-yl}-1,2-dihydro-2,7-naphthyridin-1-one. (Compound No. 144)

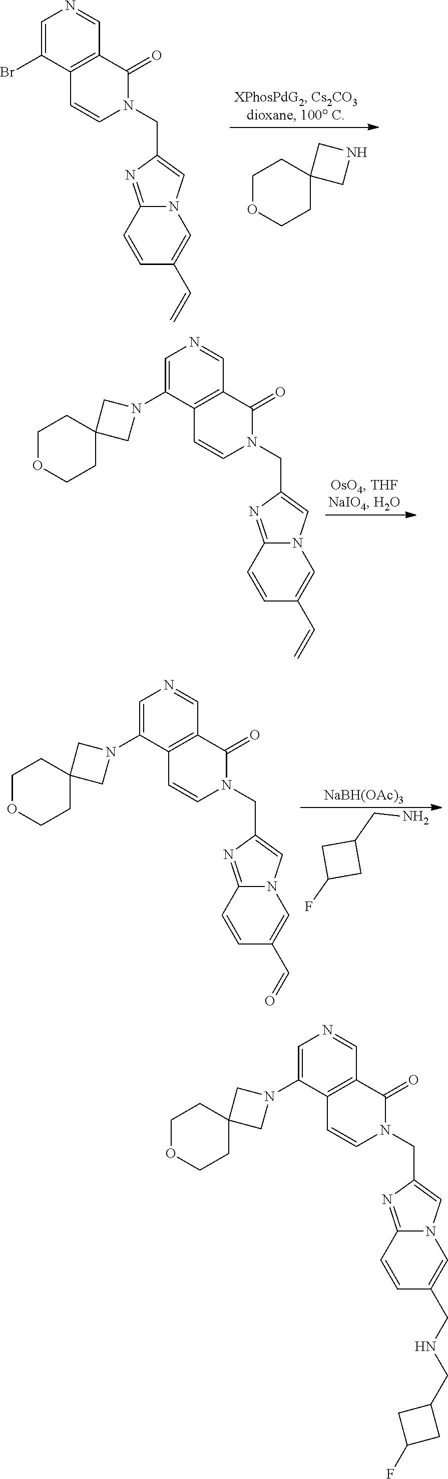

Step 1: The intermediate compound was prepared using the procedure described for example 142, step 1 with the following modification: the reaction was performed with 7-oxa-2-azaspiro[3.5]nonane to give 2-({6-ethenylimidazo[1,2-a]pyridin-2-yl}methyl)-5-{7-oxa-2-azaspiro[3.5]nonan-2-yl}-1,2-dihydro-2,7-naphthyridin-1-one (yield: 25%) as a pale yellow solid. ¹H NMR (400 MHz, DMSO-d6) δ=8.78 (s, 1H), 8.53 (s, 1H), 7.88 (s, 1H), 7.82 (s, 1H), 7.69 (d, J=7.8 Hz, 1H), 7.55-7.51 (m, 1H), 7.51-7.45 (m, 1H), 6.75-6.60 (m, 2H), 5.83 (d, J=17.6 Hz, 1H), 5.32-5.28 (m, 1H), 5.27-5.25 (m, 2H), 3.94 (s, 4H), 3.62-3.50 (m, 4H), 1.82-1.73 (m, 4H); MS: m/z 428.6 [M+H]⁺

Step 2: The intermediate compound was prepared using the procedure described for example 142, step 2 with the following modification: the reaction was performed with 2-({6-ethenylimidazo[1,2-a]pyridin-2-yl}methyl)-5-{7-oxa-2-azaspiro[3.5]nonan-2-yl}-1,2-dihydro-2,7-naphthyridin-1-one to give 2-[(5-{7-oxa-2-azaspiro[3.5]nonan-2-yl}-1-oxo-1,2-dihydro-2,7-naphthyridin-2-yl)methyl]imidazo[1,2-a]pyridine-6-carbaldehyde (yield: 65%) as a white solid. MS: m/z 429.70 [M+H]⁺

Step 3: The title compound was prepared using the procedure described for example 142, step 3 with the following modification: the reaction was performed with 2-[(5-{7-oxa-2-azaspiro[3.5]nonan-2-yl}-1-oxo-1,2-dihydro-2,7-naphthyridin-2-yl)methyl]imidazo[1,2-a]pyridine-6-carbaldehyde to give 2-{[6-(([(3-fluorocyclobutyl)methyl]amino}methyl)imidazo[1,2-a]pyridin-2-yl]methyl)-5-(7-oxa-2-azaspiro[3.5]nonan-2-yl)-1,2-dihydro-2,7-naphthyridin-1-one (yield: 13%) as an off-white solid. ¹H NMR (400 MHz, DMSO-d6) δ=8.78 (s, 1H), 8.36 (s, 1H), 8.24 (s, 1H), 7.88 (s, 1H), 7.81 (s, 1H), 7.68 (d, J=7.8 Hz, 1H), 7.43 (d, J=9.2 Hz, 1H), 7.24-7.20 (m, 1H), 6.63 (d, J=7.6 Hz, 1H), 5.25 (s, 2H), 5.19-4.98 (m, 1H), 3.93 (s, 4H), 3.64-3.62 (m, 2H), 3.57-3.54 (m, 4H), 2.60-2.53 (m, 2H), 2.24-1.94 (m, 5H), 1.79-1.76 (m, 4H); MS: m/z 517.00 [M+H]⁺

### Synthetic Example 145: Synthesis of 2-{[6-({[(3-fluorocyclobutyl)methyl]amino}methyl)imidazo[1,2-a]pyridin-2-yl]methyl}-5-{6-oxa-2-azaspiro[3.5]nonan-2-yl}-1,2-dihydro-2,7-naphthyridin-1-one. (Compound No. 145)

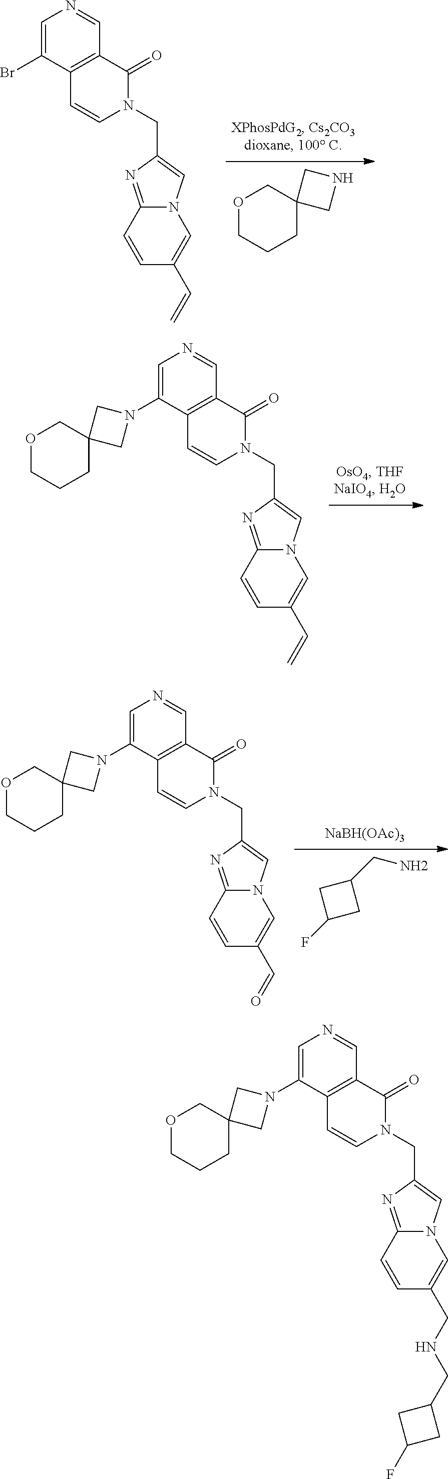

Step 1: The intermediate compound was prepared using the procedure described for example 142, step 1 with the following modification: the reaction was performed with 6-oxa-2-azaspiro[3.5]nonane to give 2-((6-ethenylimidazo[1,2-a]pyridin-2-yl)methyl)-5-(6-oxa-2-azaspiro[3.5]nonan-2-yl)-1,2-dihydro-2,7-naphthyridin-1-one (yield: 18%). ¹H NMR(400 MHz, DMSO-d6) δ=8.78 (s, 1H), 8.53 (s, 1H), 7.88 (s, 1H), 7.82 (s, 1H), 7.68 (d, J=7.9 Hz, 1H), 7.57-7.44 (m, 2H), 6.73-6.65 (m, 1H), 6.63 (d, J=7.6 Hz, 1H), 5.83 (d, J=17.9 Hz, 1H), 5.30 (d, J=11.2 Hz, 1H), 5.26 (s, 2H), 3.92 (d, J=7.6 Hz, 2H), 3.81 (d, J=7.6 Hz, 2H), 3.68 (s, 2H), 3.58-3.49 (m, 2H), 1.86-1.77 (m, 2H), 1.58-1.47 (m, 2H); MS: m/z 428.85 [M+H]⁺

Step 2: The intermediate compound was prepared using the procedure described for example 142, step 2 with the following modification: the reaction was performed with 2-({6-ethenylimidazo[1,2-a]pyridin-2-yl}methyl)-5-{6-oxa-2-azaspiro[3.5]nonan-2-yl}-1,2-dihydro-2,7-naphthyridin-1-one to get 2-[(5-{6-oxa-2-azaspiro[3.5]nonan-2-yl}-1-oxo-1,2-dihydro-2,7-naphthyridin-2-yl)methyl]imidazo[1,2-a]pyridine-6-carbaldehyde (yield: 98%) and it was used in the next step without further purification. MS: m/z 430.05 [M+H]⁺

Step 3: The title compound was prepared using the procedure described for example 142, step 3 with the following modification: the reaction was performed with 2-[(5-{6-oxa-2-azaspiro[3.5]nonan-2-yl}-1-oxo-1,2-dihydro-2,7-naphthyridin-2-yl)methyl]imidazo[1,2-a]pyridine-6-carbaldehyde to give 2-{[6-(([(3-fluorocyclobutyl)methyl]amino)methyl)imidazo[1,2-a]pyridin-2-yl]methyl}-5-{6-oxa-2-azaspiro[3.5]nonan-2-yl}-1,2-dihydro-2,7-naphthyridin-1-one (yield: 17%) as a pale yellow sticky solid. ¹H NMR (400 MHz, METHANOL-d4) δ=8.85 (s, 1H), 8.45 (s, 1H), 8.43 (br s, 1H), 7.86 (s, 1H), 7.83 (s, 1H), 7.66 (d, J=7.6 Hz, 1H), 7.53 (d, J=9.4 Hz, 1H), 7.40-7.32 (m, 1H), 6.76 (d, J=7.6 Hz, 1H), 5.34 (s, 2H), 5.18-4.96 (m, 1H), 4.03 (s, 2H), 3.99 (d, J=7.6 Hz, 2H), 3.86 (d, J=7.5 Hz, 2H), 3.79 (s, 2H), 3.68-3.60 (m, 2H), 2.93 (d, J=7.6 Hz, 2H), 2.69-2.55 (m, 1H), 2.44-2.28 (m, 2H), 2.27-2.15 (m, 2H), 1.93-1.86 (m, 2H), 1.69-1.61 (m, 2H); MS: m/z 516.85 [M+H]⁺

### Synthetic Example 146: Synthesis of 5-{7,7-difluoro-2-azaspiro[3.5]nonan-2-yl}-2-{[6-({[(3-fluorocyclobutyl)methyl]amino}methyl)imidazo[1,2-a]pyridin-2-yl]methyl}-1,2-dihydro-2,7-naphthyridin-1-one. (Compound No. 146)

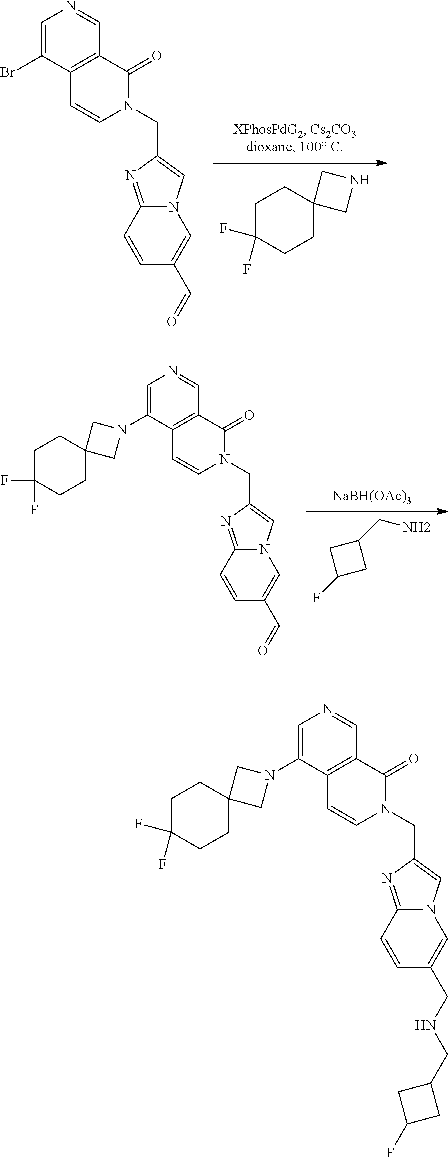

Step 1: The intermediate compound was prepared using the procedure described for example 110, step 2 with the following modification: the reaction was performed with 1-{7,7-difluoro-2-azaspiro[3.5]nonan-2-yl}-2,2,2-trifluoroethan-1-one to give 2-[(5-{7,7-difluoro-2-azaspiro[3.5]nonan-2-yl}-1-oxo-1,2-dihydro-2,7-naphthyridin-2-yl)methyl]imidazo[1,2-a]pyridine-6-carbaldehyde (yield: 12%) as a pale yellow solid. MS: m/z 463.85 [M+H]⁺

Step 2: The title compound was prepared using the procedure described for example 110, step 3 with the following modification: the reaction was performed with 2-[(5-{7,7-difluoro-2-azaspiro[3.5]nonan-2-yl}-1-oxo-1,2-dihydro-2,7-naphthyridin-2-yl)methyl]imidazo[1,2-a]pyridine-6-carbaldehyde to give 5-{7,7-difluoro-2-azaspiro[3.5]nonan-2-yl}-2-{[6-({[(3-fluorocyclobutyl)methyl]amino}methyl)imidazo[1,2-a]pyridin-2-yl]methyl}-1,2-dihydro-2,7-naphthyridin-1-one (yield: 20%) as a pale yellow solid. ¹H NMR (400 MHz, DMSO-d₆) δ=8.90 (bs, 2H), 8.79 (s, 1H), 8.64 (s, 1H), 8.07 (s, 1H), 7.87 (s, 1H), 7.86 (s, 1H), 7.64 (d, J=9.4 Hz, 1H), 7.44-7.41 (m, 1H), 6.72 (d, J=7.3 Hz, 1H), 5.34 (s, 2H), 5.24-5.01 (m, 1H), 4.22-4.12 (m, 2H), 4.07-3.87 (m, 4H), 3.09-2.94 (m, 2H), 2.65-2.56 (m, 1H), 2.38-2.10 (m, 4H), 2.05-1.79 (m, 8H); MS: m/z 551.2 [M+H]⁺

### Synthetic Example 147: Synthesis of 5-{2,2-difluoro-6-azaspiro[3.4]octan-6-yl}-2-{[6-({[(3-fluorocyclobutyl)methyl]amino}methyl)imidazo[1,2-a]pyridin-2-yl]methyl}-1,2-dihydro-2,7-naphthyridin-1-one. (Compound No. 147)

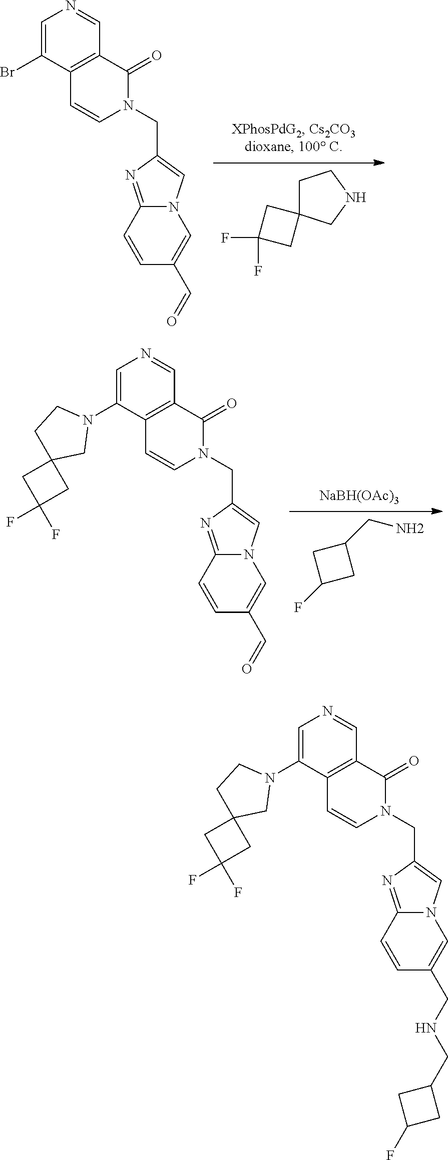

Step 1: The intermediate compound was prepared using the procedure described for example 110, step 2 with the following modification: the reaction was performed with 2,2-difluoro-6-azaspiro[3.4]octane (134 mg, 913 μmol) to give 2-[(5-{-2,2-difluoro-6-azaspiro[3.4]octan-6-yl}-1-oxo-1,2-dihydro-2,7-napththyridin-2-yl)methyl]imidazo[1,2-a]pyridine-6-carbaldehyde (yield: 5%) as a white solid. MS: m/z 449.85 [M+H]⁺

Step 2: The title compound was prepared using the procedure described for example 110, step 3 with the following modification: the reaction was performed with 5-{2,2-difluoro-60 azaspiro[3.4]octan-6-yl}-2-{[6-({[(3-fluorocyclobutyl)methyl]amino}methyl)imidazo[1,2-a]pyridin-2-yl]methyl}-1,2-dihydro-2,7-napththyridin-1-one to give 5-{2,2-difluoro-6-azaspiro[3.4]octan-6-yl}-2-{[6-({[(3-fluorocyclobutyl)methyl]amino}methyl)imidazo[1,2-a]pyridin-2-yl]methyl}-1,2-dihydro-2,7-naphthyridin-1-one (yield: 11%) as an off white solid ¹H NMR (400 MHz, METHANOL-d4) δ=8.83 (s, 1H), 8.32 (s, 1H), 8.03 (s, 1H), 7.76 (s, 1H), 7.58 (d, J=7.6 Hz, 1H), 7.42 (d, J=9.4 Hz, 1H), 7.29-7.25 (m, 1H), 6.85 (d, J=8.2 Hz, 1H), 5.25 (s, 2H), 5.08-4.86 (m, 1H), 3.86 (s, 2H), 3.56-3.45 (m, 4H), 2.78-2.71 (m, 2H), 2.62-2.49 (m, 3H), 2.61-2.47 (m, 2H), 2.33-2.15 (m, 2H), 2.12-2.05 (in, 4H); MS: m/z 537.25 [M+H]⁺

### Synthetic Example 148: Synthesis of 2-{[6-({[(3-fluorocyclobutyl)methyl]amino}methyl)imidazo[1,2-a]pyridin-2-yl]methyl}-5-[3-(4-fluoropiperidin-1-yl)azetidin-1-yl]-1,2-dihydro-2,7-naphthyridin-1-one. (Compound No. 148)

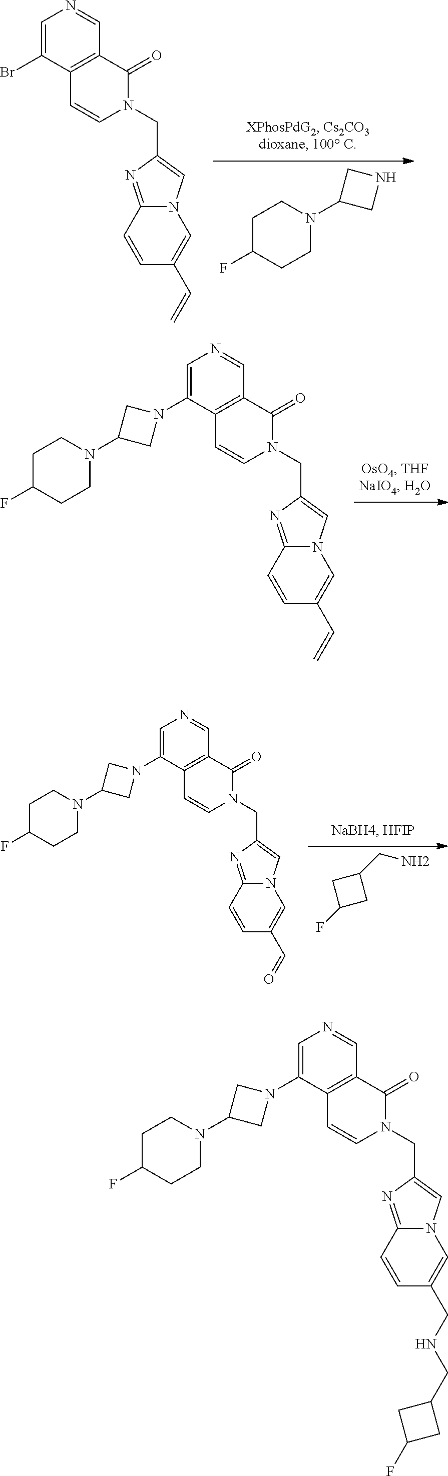

Step 1: The intermediate compound was prepared using the procedure described for example 142, step 1 with the following modification: the reaction was performed with 1-(azetidin-3-yl)-4-fluoropiperidine to give 2-({6-ethenylimidazo[1,2-a]pyridin-2-yl}methyl)-5-[3-(4-fluoropiperidin-1-yl)azetidin-1-yl]-1,2-dihydro-2,7-naphthyridin-1-one (yield: 7%). ¹H NMR (400 MHz, DMSO-d6) δ=8.80 (s, 1H), 8.53 (s, 1H), 7.90 (s, 1H), 7.83 (s, 1H), 7.69 (d, J=7.6 Hz, 1H), 7.57-7.51 (m, 1H), 7.50-7.46 (m, 1H), 6.74-6.64 (m, 1H), 6.61 (d, J=7.6 Hz, 1H), 5.83 (d, J=17.6 Hz, 1H), 5.30 (d, J=11.2 Hz, 1H), 5.26 (s, 2H), 4.80-4.59 (m, 1H), 4.25 (t, J=7.3 Hz, 2H), 3.93-3.84 (m, 2H), 3.29-3.23 (m, 1H), 2.05-1.94 (m, 1H), 1.92-1.78 (m, 21), 1.78-1.66 (m, 2H); MS: m/z 458.65 [M+H]⁺

Step 2: The intermediate compound was prepared using the procedure described for example 142, step 2 with the following modification: the reaction was performed with 2-({6-ethenylimidazo[1,2-a]pyridin-2-yl}methyl)-5-[3-(4-fluoropiperidin-1-yl)azetidin-1-yl]-1,2-dihydro-2,7-naphthyridin-1-one to give 2-({5-[3-(4-fluoropiperidin-1-yl)azetidin-1-yl]-1-oxo-1,2-dihydro-2,7-naphthyridin-2-yl}methyl)imidazo[1,2-a]pyridine-6-carbaldehyde (yield: 40%) as an off-white solid. MS: m/z 460.90 [M+H]⁺

Step 3: The title compound was prepared using the procedure described for example 142, step 3 with the following modification: the reaction was performed with 2-({5-[3-(4-fluoropiperidin-1-yl)azetidin-1-yl]-1-oxo-1,2-dihydro-2,7-naphthyridin-2-yl}methyl)imidazo[1,2-a]pyridine-6-carbaldehyde to give 2-{[6-({[(3-fluorocyclobutyl)methyl]amino}methyl)imidazo[1,2-a]pyridin-2-yl]methyl}-5-[3-(4-fluoropiperidin-1-yl)azetidin-1-yl]-1,2-dihydro-2,7-naphthyridin-1-one (yield: 22%) as a yellow sticky solid. ¹H NMR (400 MHz, METHANOL-d4) δ=8.98 (s, 1H), 8.64 (s, 1H), 8.05 (s, 1H), 7.96 (s, 1H), 7.91 (d, J=7.6 Hz, 1H), 7.65 (d, J=9.2 Hz, 1H), 7.51 (d, J=9.5 Hz, 1H), 6.76 (d, J=7.6 Hz, 1H), 5.42 (s, 2H), 5.23-5.12 (m, 1H), 5.07-5.01 (m, 1H), 4.95-4.90 (m, 1H), 4.65-4.55 (m, 2H), 4.50-4.40 (m, 2H), 4.27 (s, 4H), 3.46-3.37 (m, 2H), 3.19 (d, J=7.8 Hz, 2H), 2.82-2.66 (m, 1H), 2.54-2.35 (m, 2H), 2.34-2.06 (m, 6H); MS: m/z 548.3 [M+H]⁺

### Synthetic Example 149: Synthesis of 2-{[6-({[(3-fluorocyclobutyl)methyl]amino}methyl)imidazo[1,2-a]pyridin-2-yl]methyl}-5-[3-(pyridin-4-yl)azetidin-1-yl]-1,2-dihyro-2,7-naphthyridin-1-one. (Compound No. 149)

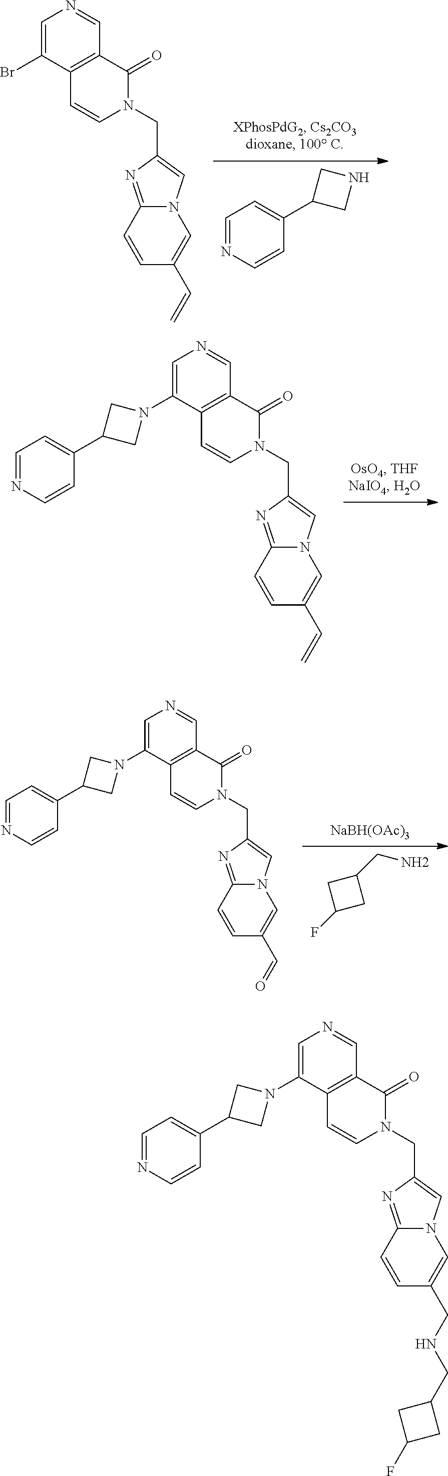

Step 1: The intermediate compound was prepared using the procedure described for example 142, step 1 with the following modification: the reaction was performed with 4-(azetidin-3-yl)pyridine to give 2-({6-ethenylimidazo[1,2-a]pyridin-2-yl}methyl)-5-[3-(pyridin-4-yl)azetidin-1-yl]-1,2-dihydro-2,7-naphthyridin-1-one (yield: 32%) as a pale yellow solid. MS: m/z 434.90 [M+H]⁺

Step 2: The intermediate compound was prepared using the procedure described for example 142, step 2 with the following modification: the reaction was performed with 2-({6-ethenylimidazo[1,2-a]pyridin-2-yl}methyl)-5-[3-(pyridin-4-yl)azetidin-1-yl]-1,2-dihydro-2,7-naphthyridin-1-one to give 2-({1-oxo-5-[3-(pyridin-4-yl)azetidin-1-yl]-1,2-dihydro-2,7-naphthyridin-2-yl}methyl)imidazo[1,2-a]pyridine-6-carbaldehyde (yield: 87%) as a white solid. ¹H NMR (400 MHz, DMSO-d6) δ=9.90 (s, 1H), 9.26 (s, 1H), 8.86 (br s, 1H), 8.55 (br s, 2H), 8.09 (s, 1H), 8.04-7.95 (m, 1H), 7.74 (d, J=7.6 Hz, 1H), 7.64-7.55 (m, 2H), 7.47 (br d, J=5.1 Hz, 2H), 6.67 (br d, J=7.6 Hz, 1H), 5.33 (s, 2H), 4.57 (brt, J=7.9 Hz, 2H), 4.17 (brt, J=6.7 Hz, 2H), 4.09-3.95 (m, 1H); MS: m/z 436.90 [M+H]⁺

Step 3: The title compound was prepared using the procedure described for example 142, step 3 with the following modification: the reaction was performed with 2-({1-oxo-5-[3-(pyridin-4-yl)azetidin-1-yl]-1,2-dihydro-2,7-naphthyridin-2-yl}methyl)imidazo[1,2-a]pyridine-6-carbaldehyde to give 2-{[6-({[(3-fluorocyclobutyl)methyl]amino}methyl)imidazo[1,2-a]pyridin-2-yl]methyl}-5-[3-(pyridin-4-yl)azetidin-1-yl]-1,2-dihydro-2,7-naphthyridin-1-one (yield: 15%) as a pale brown solid. ¹H NMR (400 MHz, DMSO-d6) δ=8.85 (s, 1H), 8.54 (br d, J=5.7 Hz, 2H), 8.37 (s, 1H), 8.29 (br s, 1H), 7.98 (s, 1H), 7.83 (s, 1H), 7.71 (d, J=7.6 Hz, 1H), 7.45-7.40 (m, 2H), 7.52-7.39 (m, 1H), 7.27-7.18 (m, 1H), 6.65 (d, J=7.8 Hz, 1H), 5.26 (s, 2H), 5.19-4.98 (m, 1H), 4.56 (t, J=7.9 Hz, 2H), 4.16 (t, J=6.7 Hz, 2H), 4.10-3.93 (m, 2H), 2.48-46 (m, 2H), 2.41-2.27 (m, 2H), 2.27-2.00 (m, 4H); MS: m/z 523.95 [M+H]⁺

### Synthetic Example 150: Synthesis of 2-{[6-({[(3-fluorocyclobutyl)methyl]amino}methyl)imidazo[1,2-a]pyridin-2-yl]methyl}-5-[3-(4-fluorophenyl)azetidin-1-yl]-1,2-dihydro-2,7-naphthyridin-1-one. (Compound No. 150)

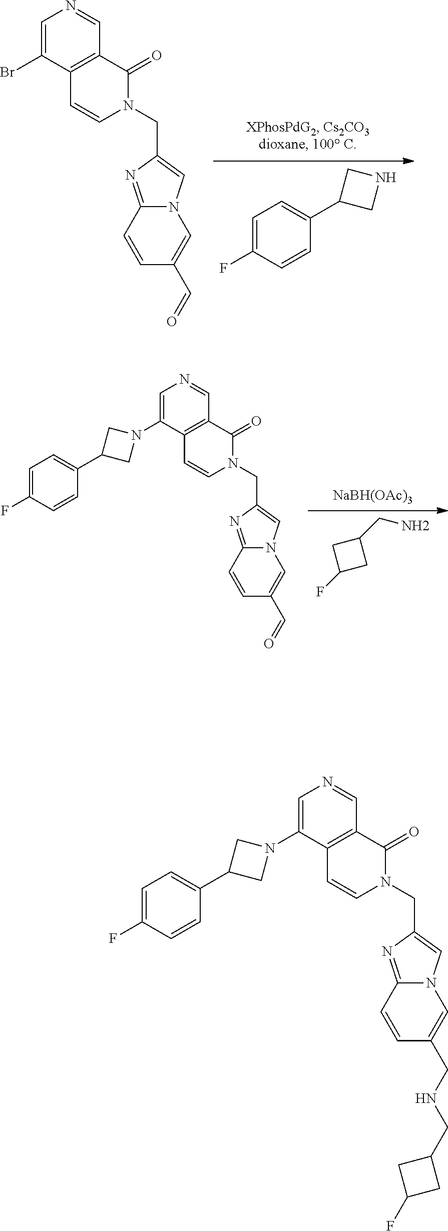

Step 1: The intermediate compound was prepared using the procedure described for example 110, step 2 with the following modification: the reaction was performed with 3-(4-fluorophenyl)azetidine to give 2-({5-[3-(4-fluorophenyl)azetidin-1-yl]-1-oxo-1,2-dihydro-2,7-naphthyridin-2-yl}methyl)imidazo[1,2-a]pyridine-6-carbaldehyde (yield: 11%) as a white solid MS: m/z 453.65 [M+H]⁺

Step 2: The title compound was prepared using the procedure described for example 110, step 3 with the following modification: the reaction was performed with bis(2-({5-[3-(4-fluorophenyl)azetidin-1-yl]-1-oxo-1,2-dihydro-2,7-naphthyridin-2-yl}methyl)imidazo[1,2-a]pyridine-6-carbaldehyde) to give bis(2-{[6-({[(3-fluorocyclobutyl)methyl]amino}methyl)imidazo[1,2-a]pyridin-2-yl]methyl}-5-[3-(4-fluorophenyl)azetidin-1-yl]-1,2-dihydro-2,7-naphthyridin-1-one) (yield: 11%) as a white solid. ¹H NMR (400 MHz, METHANOL-d4) δ=8.92 (s, 1H), 8.34 (s, 1H), 7.92 (s, 1H), 7.82 (s, 1H), 7.66 (d, J=7.8 Hz, 1H), 7.52-7.43 (m, 3H), 7.36 (d, J=11.0 Hz, 1H), 7.09 (t, J=8.8 Hz, 2H), 6.78 (d, J=7.3 Hz, 1H), 5.35 (s, 2H), 5.15-4.92 (m, 1H), 4.63 (t, J=7.8 Hz, 2H), 4.22-4.14 (m, 2H), 4.09-4.01 (m, 1H), 3.78 (s, 2H), 2.65 (d, J=7.6 Hz, 2H), 2.54-2.45 (m, 1H), 2.38-2.21 (m, 2H), 2.20-2.08 (m, 2H); MS: m/z 540.90 [M+H]⁺

### Synthetic Example 151: Synthesis of 2-((6-((((3-fluorobicyclo[1.1.1]pentan-1-yl)methyl)amino)methyl)imidazo[1,2-a]pyridin-2-yl)methyl)-5-(6-oxa-2-azaspiro[3.4]octan-2-yl)-2,7-naphthyridin-1(2H)-one. (Compound No. 151)

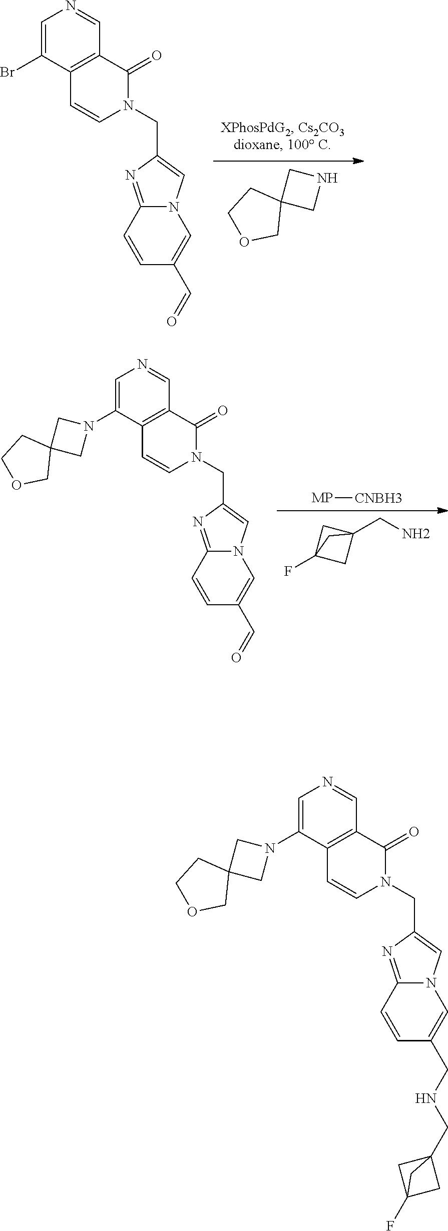

Step 1: The intermediate compound was prepared using the procedure described for example 110, step 2 with the following modification: the reaction was performed with 6-oxa-2-azaspiro[3.4]octane to afford 2-({6-ethenylimidazo[1,2-a]pyridin-2-yl}methyl)-5-{6-oxa-2-azaspiro[3.4]octan-2-yl}-1,2-dihydro-2,7-naphthyridin-1-one (yield: 52%) as a pale yellow solid. MS: m/z 414.10 [M+H]⁺

Step 2: To a stirred solution of 2-[(5-{6-oxa-2-azaspiro[3.4]octan-2-yl}-1-oxo-1,2-dihydro-2,7-naphthyridin-2-yl)methyl]imidazo[1,2-a]pyridine-6-carbaldehyde (70 mg, 168 μmol) and 1-{3-fluorobicyclo[1.1.1]pentan-1-yl}methanamine(97 mg, 840 μmol) in DCM:MeOH (2:1) (3 mL) was added add acetic acid (2.53 mg, 42.1 μmol) and after 3 h at room temperature, resin MP-CNBH₃ (50 mg) added and the reaction mixture was stirred at room temperature for 12 h. The reaction mixture was extracted with DCM (10 mL×3) and washed with saturated NaHCO₃ solution and saturated brine solution. The combined organic layers were dried over Na₂SO₄, filtered, concentrated in vacuo and purified by preparative HPLC (WATERS×BRIDGE (150 mm×20.0 mm), Phase A: 0.02% NH₄OH in H₂O, Phase B: ACN, gradient Elution) to give 2-[(6-{[({3-fluorobicyclo[1.1.1]pentan-1-yl}methyl)amino]methyl}imidazo[1,2-a]pyridin-2-yl)methyl]-5-{6-oxa-2-azaspiro[3.4]octan-2-yl}-1,2-dihydro-2,7-naphthyridin-1-one (15 mg, yield: 11%) as a light yellow sticky compound. ¹H NMR (400 MHz, METHANOL-d4) δ=8.90 (s, 1H), 8.33 (s, 1H), 7.86 (s, 1H), 7.81 (s, 1H), 7.66 (d, J=7.8 Hz, 1H), 7.47 (d, J=7.4 Hz, 1H), 7.39-7.33 (m, 1H), 6.74 (d, J=7.6 Hz, 1H), 5.34 (s, 2H), 4.18 (s, 4H), 3.95 (s, 2H), 3.87 (t, J=7.0 Hz, 2H), 3.78 (s, 2H), 2.86 (s, 2H), 2.26 (t, J=7.0 Hz, 2H), 1.97 (d, J=2.6 Hz, 6H); MS: m/z 515.3 [M+H]⁺

### Synthetic Example 152: Synthesis of 2-[(6-{[({3-fluorobicyclo[1.1.1]pentan-1-yl}methyl)amino]methyl}imidazo[1,2-a]pyridin-2-yl)methyl]-5-{7-oxa-2-azaspiro[3.5]nonan-2-yl}-1,2-dihydro-2,7-naphthyridin-1-one. (Compound No. 152)

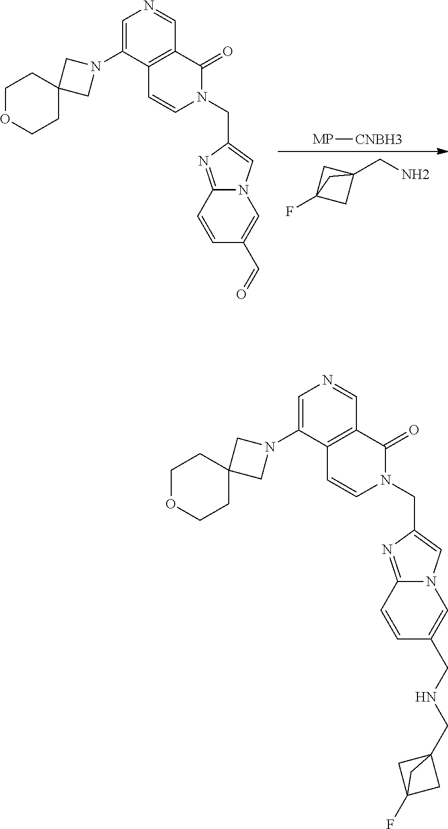

The title compound was prepared using the procedure described for example 151, step 2 with the following modification: the reaction was performed with 2-[(5-{7-oxa-2-azaspiro[3.5]nonan-2-yl}-1-oxo-1,2-dihydro-2,7-naphthyridin-2-yl)methyl]imidazo[1,2-a]pyridine-6-carbaldehyde to give 2-[(6-([({3-fluorobicyclo[1.1.1]pentan-1-yl)methyl)amino]methyl}imidazo[1,2-a]pyridin-2-yl)methyl]-5-{7-oxa-2-azaspiro[3.5]nonan-2-yl}-1,2-dihydro-2,7-naphthyridin-1-one (yield: 12%) as a light brown solid. ¹H NMR (400 MHz, DMSO-d6) δ=8.78 (s, 1H), 8.36 (s, 1H), 7.88 (s, 1H), 7.81 (s, 1H), 7.68 (d, J=7.6 Hz, 1H), 7.43 (d, J=9.2 Hz, 1H), 7.26-7.15 (m, 1H), 6.64 (d, J=7.8 Hz, 1H), 5.25 (s, 2H), 3.94 (s, 4H), 3.65 (s, 2H), 3.60-3.51 (m, 4H), 2.72 (s, 2H), 1.92 (d, J=2.6 Hz, 6H), 1.77 (br t, J=4.9 Hz, 4H); MS: m/z 529.3 [M+H]⁺

### Synthetic Example 153: Synthesis of 2-[(6-{[({3-fluorobicyclo[1.1.1]pentan-1-yl}methyl)amino]methyl}imidazo[1,2-a]pyridin-2-yl)methyl]-5-{6-oxa-2-azaspiro[3.5]nonan-2-yl}-1,2-dihydro-2,7-naphthyridin-1-one. (Compound No. 153)

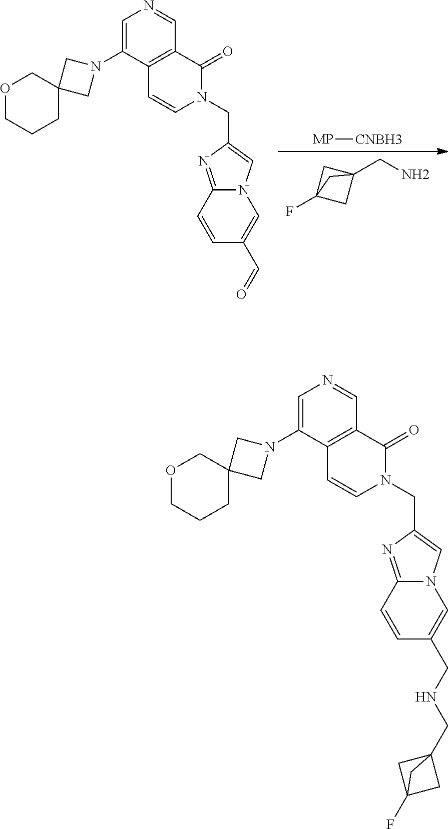

The title compound was prepared using the procedure described for example 151, step 2 with the following modification: the reaction was performed with 2-[(5-{6-oxa-2-azaspiro[3.5]nonan-2-yl}-1-oxo-1,2-dihydro-2,7-naphthyridin-2-yl)methyl]imidazo[1,2-a]pyridine-6-carbaldehyde to give 2-[(6-([({3-fluorobicyclo[1.1.1]pentan-1-yl}methyl)amino]methyl)imidazo[1,2-a]pyridin-2-yl)methyl]-5-{6-oxa-2-azaspiro[3.5]nonan-2-yl}-1,2-dihydro-2,7-naphthyridin-1-one (yield: 20%) as an off-white solid. ¹H NMR (400 MHz, DMSO-d6) δ=8.78 (s, 1H), 8.66 (s, 1H), 8.09 (s, 1H), 7.94 (d, J=7.6 Hz, 1H), 7.83 (br s, 1H), 7.67 (d, J=9.4 Hz, 1H), 7.50 (dd, J=9.4, 1.7 Hz, 1H), 6.82 (d, J=7.6 Hz, 1H), 5.38 (s, 2H), 4.20 (s, 2H), 4.02 (d, J=7.9 Hz, 2H), 3.93 (d, J=8.1 Hz, 2H), 3.63 (s, 2H), 3.55 (br t, J=4.5 Hz, 2H), 3.32 (s, 2H), 2.11 (d, J=2.2 Hz, 6H), 1.85 (br t, J=5.4 Hz, 2H), 1.58-1.50 (m, 2H); MS: m/z 529.05 [M+H]⁺

### Synthetic Example 154: Synthesis of 2-[(6-{[({3-fluorobicyclo[1.1.1]pentan-1-yl}methyl)amino]methyl}imidazo[1,2-a]pyridin-2-yl)methyl]-5-(2-fluorophenyl)-1,2-dihydro-2,7-naphthyridin-1-one (Compound No. 154)

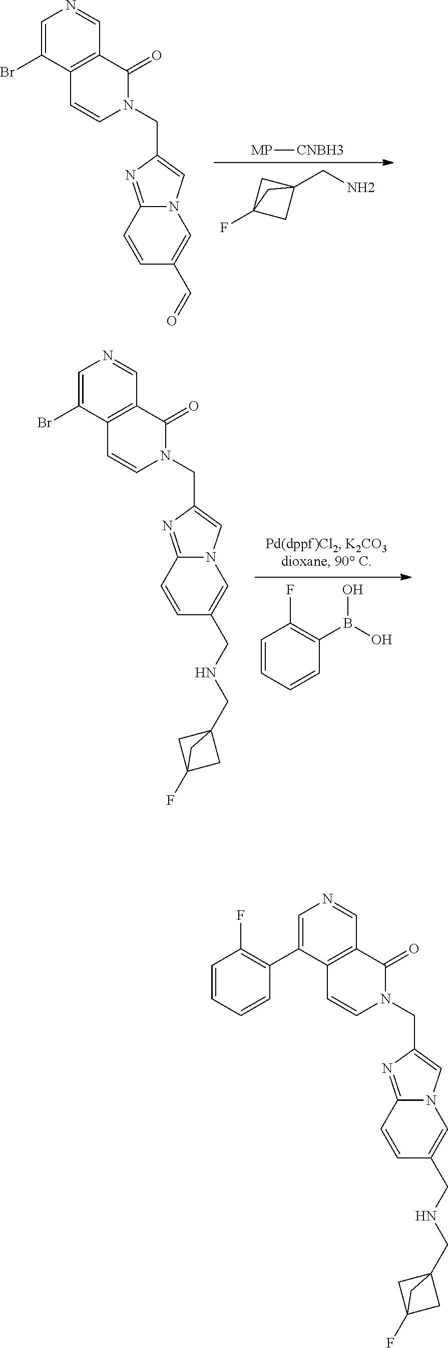

Step 1: The intermediate compound was prepared using the procedure described for example 151, step 2 with the following modification: the reaction was performed with 2-[(5-bromo-1-oxo-1,2-dihydro-2,7-naphthyridin-2-yl)methyl]imidazo[1,2-a]pyridine-6-carbaldehyde to give 5-bromo-2-[(6-{[({3-fluorobicyclo[1.1.1]pentan-1-yl}methyl)amino]methyl}imidazo[1,2-a]pyridin-2-yl)methyl]-1,2-dihydro-2,7-naphthyridin-1-one (yield: 32%) as an off white solid. ¹H NMR(400 MHz, DMSO-d6) δ=9.29 (s, 1H), 8.94 (s, 1H), 8.36 (s, 1H), 8.04 (d, J=7.5 Hz, 1H), 7.85 (s, 1H), 7.43 (d, J=9.2 Hz, 1H), 7.22 (d, J=10.9 Hz, 1H), 6.71 (d, J=7.5 Hz, 1H), 5.30 (s, 2H), 3.66 (s, 2H), 2.72 (s, 2H), 1.92 (d, J=2.6 Hz, 6H); MS: m/z 484.10 [M+H]⁺

Step 2: A stirred solution of 5-bromo-2-[(6-{[({3-fluorobicyclo[1.1.1]pentan-1-yl}methyl)amino]methyl}imidazo[1,2-a]pyridin-2-yl)methyl]-1,2-dihydro-2,7-naphthyridin-1-one (40 mg, 82.9 μmol), (2-fluorophenyl)boronic acid (23.2 mg, 166 μmol) and K₂CO₃ (34.3 mg, 249 μmol) in a 2:1mixture of Dioxane:H₂O (2 mL) was purged under N₂ gas for 10 minutes followed by the addition of PdCl₂(dppf)DCM (6.77 mg, 8.29 μmol). The reaction mixture was heated at 80° C. for 2 h, cooled to room temperature and partitioned between EtOAc and water (10 mL). The aqueous layer was extracted with EtOAc (20 mL×2) and the combined organic layers were dried over Na₂SO₄, filtered, concentrated in vacuo and purified by flash column chromatography (SiO₂, 0-10% MeOH in DCM) to afford 2-[(6-([({3-fluorobicyclo[1.1.1]pentan-1-yl}methyl)amino]methyl)imidazo[1,2-a]pyridin-2-yl)methyl]-5-(2-fluorophenyl)-1,2-dihydro-2,7-naphthyridin-1-one (14.6 mg, yield: 35%) as a yellow solid. ¹H NMR (400 MHz, DMSO-d6) δ=9.42 (s, 1H), 8.68 (s, 1H), 8.36 (s, 1H), 7.90-7.81 (m, 2H), 7.65-7.55 (m, 1H), 7.52-7.47 (m, 1H), 7.45-7.37 (m, 3H), 7.22 (d, J=9.2 Hz, 1H), 6.21 (br d, J=9.4 Hz, 1H), 5.29 (s, 2H), 3.65 (s, 2H), 2.72 (s, 2H), 1.92 (d, J=2.6 Hz, 6H); MS: m/z 497.50 [M+H]⁺

### Synthetic Example 155: Synthesis of 4-{7-[(6-{[({3-fluorobicyclo[1.1.1]pentan-1-yl}methyl)amino]methyl}imidazo[1,2-a]pyridin-2-yl)methyl]-8-oxo-7,8-dihydro-2,7-naphthyridin-4-yl}benzamide (Compound No. 155)

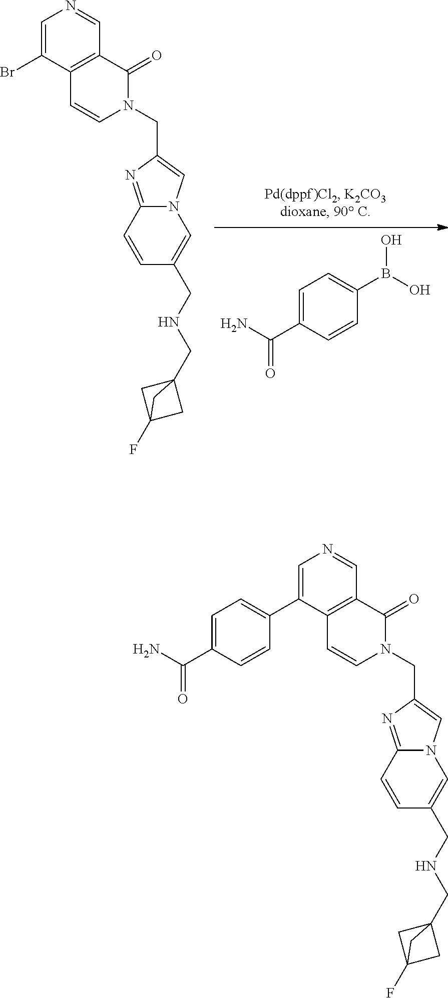 [SMILES: O=c1c2cncc(Br)c2ccn1Cc1cn2cc(CNCC34CC(F)(C3)C4)ccc2n1 ; NC(=O)c1ccc(B(O)O)cc1 ; NC(=O)c1ccc(-c2cncc3c(=O)n(Cc4cn5cc(CNCC67CC(F)(C6)C7)ccc5n4)ccc23)cc1]

The title compound was prepared using the procedure described for example 154, step 2 with the following modification: the reaction was performed with (4-carbamoylphenyl)boronic acid to give 4-{7-[(6-([({3-fluorobicyclo[1.1.1]pentan-1-yl}methyl)amino]methyl}imidazo[1,2-a]pyridin-2-yl)methyl]-8-oxo-7,8-dihydro-2,7-naphthyridin-4-yl)benzamide (yield: 35%) as a yellow solid. ¹H NMR (400 MHz, DMSO-d6) δ=9.41 (s, 1H), 8.69 (s, 1H), 8.36 (s, 1H), 8.10 (br s, 1H), 8.04 (d, J=8.2 Hz, 2H), 7.90-7.86 (m, 2H), 7.58 (d, J=8.4 Hz, 2H), 7.47 (br s, 1H), 7.43 (d, J=9.2 Hz, 1H), 7.22 (d, J=9.2 Hz, 1H), 6.47 (d, J=7.6 Hz, 1H), 5.30 (s, 2H), 3.66 (s, 2H), 2.73 (s, 3H), 1.92 (d, J=2.6 Hz, 6H); MS: m/z 524.25 [M+H]⁺

### Synthetic Example 156: Synthesis of 5-cyclobutyl-2-{[6-({[(3-fluorocyclobutyl)methyl]amino}methyl)imidazo[1,2-a]pyridin-2-yl]methyl}-1,2-dihydro-2,7-naphthyridin-1-one. (Compound No. 156)

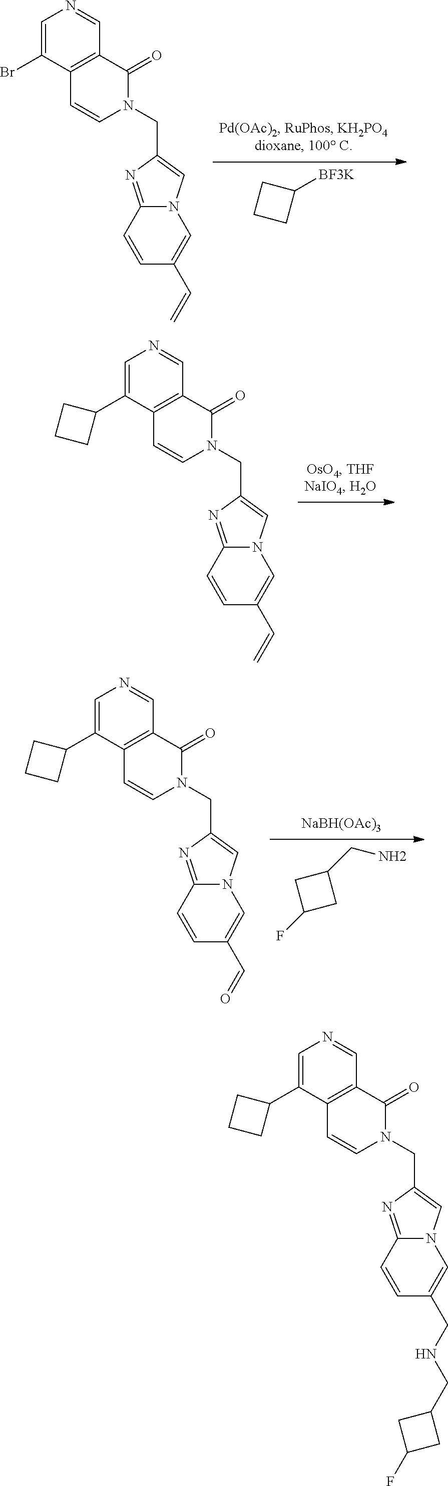

Step 1: A solution of 5-bromo-2-({6-ethenylimidazo[1,2-a]pyridin-2-yl}methyl)-1,2-dihydro-2,7-naphthyridin-1-one (100 mg, 262 μmol), cyclobutyltrifluoro-λ⁴-borane potassium (127 mg, 787 μmol), and KH₂PO₄ (37.5 mg, 275 μmol) in toluene (5 mL) and water (1 mL) was purged with nitrogen for 10 min, followed by the addition of Pd(OAc)₂(1.18 mg, 5.25 μmol) and Ruphos (6.12 mg, 13.1 μmol). The reaction mixture was heated at 85° C. for 1 h, cooled to room temperature, diluted with water (10 ml), and extracted with MeOH in DCM (3×20 ml). The combined organic layers were washed with brine solution, dried over Na₂SO₄, filtered, concentrated in vacuo and purified by flash chromatography (SiO₂, 5%-15% MeOh in DCM) to give 5-cyclobutyl-2-({6-ethenylimidazo[1,2-a]pyridin-2-yl}methyl)-1,2-dihydro-2,7-naphthyridin-1-one (25 mg, yield: 27%) as an off white solid. MS: m/z 356.90 [M+H]⁺

Step 2: The intermediate compound was prepared using the procedure described for example 111, step 4 with the following modification: the reaction was performed with 5-cyclobutyl-2-({6-ethenylimidazo[1,2-a]pyridin-2-yl}methyl)-1,2-dihydro-2,7-naphthyridin-1-one to give 2-[(5-cyclobutyl-1-oxo-1,2-dihydro-2,7-naphthyridin-2-yl)methyl]imidazo[1,2-a]pyridine-6-carbaldehyde (yield: 19%) as a brown solid which was used in the next step without further purification. MS: m/z 359.05 [M+H]⁺

Step 3: The title compound was prepared using the procedure described for example 142, step 3 with the following modification: the reaction was performed with 2-[(5-cyclobutyl-1-oxo-1,2-dihydro-2,7-naphthyridin-2-yl)methyl]imidazo[1,2-a]pyridine-6-carbaldehyde to afford 5-cyclobutyl-2-{[6-({[(3-fluorocyclobutyl)methyl]amino}methyl)imidazo[1,2-a]pyridin-2-yl]methyl}-1,2-dihydro-2,7-naphthyridin-1-one (yield: 9%) as a clear oil ¹H NMR (400 MHZ, METHANOL-d4) δ=9.37 (s, 1H), 8.66 (s, 1H), 8.56 (s, 1H), 8.07 (s, 1H), 8.05 (d, J=7.6 Hz, 1H), 7.67 (d, J=9.4 Hz, 1H), 7.54 (d, J=9.2 Hz, 1H), 6.84 (d, J=7.6 Hz, 1H), 5.45 (s, 2H), 5.25-5.00 (m, 1H), 4.28 (s, 2H), 3.19 (d, J=7.8 Hz, 2H), 2.81-2.67 (m, 1H), 2.63-2.51 (m, 2H), 2.50-2.35 (m, 2H), 2.35-2.19 (m, 5H), 2.05-1.91 (m, 2H); MS: m/z 446.20 [M+H]⁺

### Synthetic Example 157: Synthesis of 3-[(6-{[({3-fluorobicyclo[1.1.1]pentan-1-yl}methyl)amino]methyl}imidazo[1,2-a]pyridin-2-yl)methyl]-8-{2-oxa-6-azaspiro[3.3]heptan-6-yl}pyrido[3,4-d]pyridazin-4-one. (Compound No. 157)

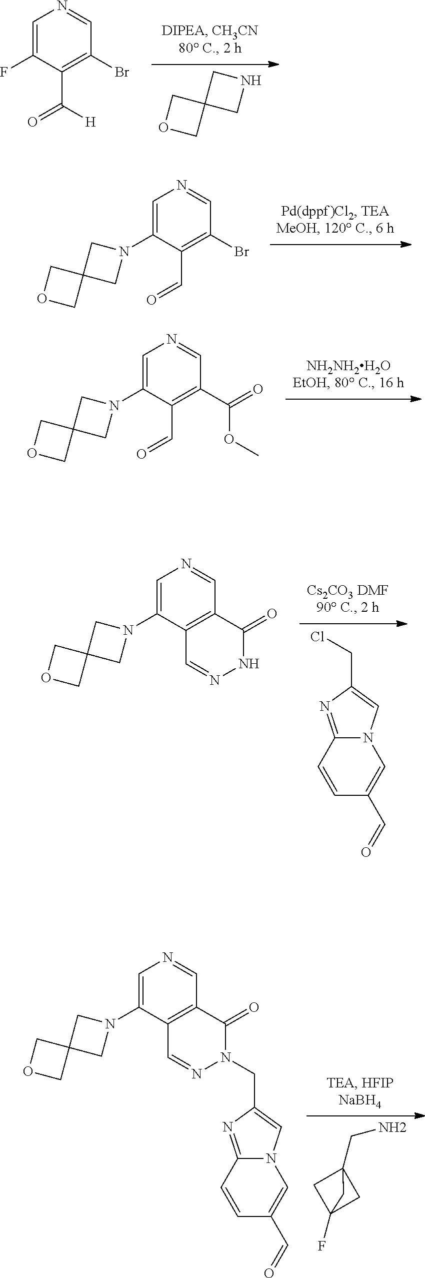

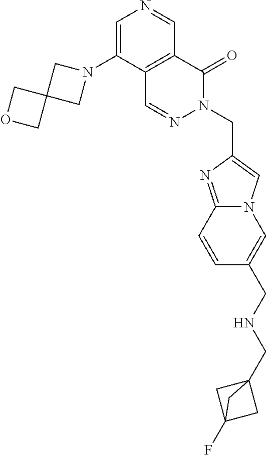 [SMILES: O=c1c2cncc(N3CC4(COC4)C3)c2cnn1Cc1cn2cc(CNCC34CC(F)(C3)C4)ccc2n1]

Step 1: To a stirred solution of 3-bromo-5-fluoropyridine-4-carbaldehyde (3 g, 14.71 mmol) and 2-oxa-6-azaspiro[3.3]heptane (1.46 g 14.71 mmol) in ACN (40 mL) was added DIPEA(3.80 g 29.41 mmol) in portions at room temperature and the resulting mixture was heated at 80° C. for 2 h. The reaction mixture was cooled to room temperature, diluted with water (20 mL) and extracted with EtOAc (3×100 mL). The combined organic layers were washed with brine(2×100 mL), dried over Na₂SO₄, filtered, concentrated in vacuo and purified by flash chromatography (SiO₂, 30%-40% of EtOAc/petroleum ether) to afford 3-bromo-5-{2-oxa-6-azaspiro[3.3]heptan-6-yl}pyridine-4-carbaldehyde(2.5 g, yield: 60%) as a brown solid. MS: m/z=283.0 [M+H]⁺

Step 2: To a stirred mixture of 3-bromo-5-{2-oxa-6-azaspiro[3.3]heptan-6-yl}pyridine-4-carbaldehyde (2.4 g 8.48 mmol) in MeOH (40 mL) was added Pd(dppf)Cl₂(0.62 g, 0.85 mmol) and TEA (2.57 g, 25.43 mmol) in portions at room temperature. The reaction mixture was heated at 120° C. for 6 h under a carbon monoxide atmosphere. The reaction mixture was cooled to room temperature, concentrated in vacuo and purified by flash column chromatography (SiO₂, 40%-50% of EtOAc/petroleum ether) to afford methyl 4-formyl-5-{2-oxa-6-azaspiro[3.3]heptan-6-yl}pyridine-3-carboxylate (1.6 g, yield: 72%) as a yellow solid. MS: m/z=263.0 [M+H]⁺

Step 3: To a stirred solution of methyl 4-formyl-5-{2-oxa-6-azaspiro[3.3]heptan-6-yl}pyridine-3-carboxylate (800 mg, 3.05 mmol) in EtOH (5 mL) was added hydrazine hydrate (20 mL) in portions at room temperature. The resulting mixture was heated at 80° C. for 16 h, cooled to room temperature, diluted with H₂O (10 mL) and extracted with EtOAc (3×30 mL). The combined organic layers were washed with brine (2×30 mL), dried over Na₂SO₄, filtered, concentrated in vacuo, and purified by flash column chromatography (SiO₂, 50%-60% of EtOAc/petroleum ether) to afford 8-{2-oxa-6-azaspiro[3.3]heptan-6-yl}-3H-pyrido[3,4-d]pyridazin-4-one (430 mg, yield: 58%) as a yellow solid. MS: m/z=245.0 [M+H]⁺

Step 4: The intermediate compound was prepared using the procedure described for example 110, step 1 with the following modification: the reaction was performed with 8-{2-oxa-6-azaspiro[3.3]heptan-6-yl}-3H-pyrido[3,4-d]pyridazin-4-one to give 2-[(8-{2-oxa-6-azaspiro[3.3]heptan-6-yl}-4-oxopyrido[3,4-d]pyridazin-3-yl)methyl]imidazo[1,2-a]pyridine-6-carbaldehyde (yield: 40%) as a brown solid. MS: m/z=403.0 [M+H]⁺

Step 5: The title compound was prepared using the procedure described for example 110, step 3 with the following modification: the reaction was performed with 2-[(8-{2-oxa-6-azaspiro[3.3]heptan-6-yl}-4-oxopyrido[3,4-d]pyridazin-3-yl)methyl]imidazo[1,2-a]pyridine-6-carbaldehyde and 1-{3-fluorobicyclo[1.1.1]pentan-1-yl}methanamine hydrochloride to afford 3-[(6-{[({3-fluorobicyclo[1.1.1]pentan-1-yl}methyl)amino]methyl}imidazo[1,2-a]pyridin-2-yl)methyl]-8-{2-oxa-6-azaspiro[3.3]heptan-6-yl}pyrido[3,4-d]pyridazin-4-one (yield: 2%) as a yellow solid. ¹H NMR (400 MHz, Chloroform-d) δ 9.06 (s, 1H), 8.25 (s, 1H), 8.06 (s, 1H), 8.03 (s, 1H), 7.65 (s, 1H), 7.50 (d, J=9.2 Hz, 1H), 7.09 (d, J=9.2 Hz, 1H), 5.53 (s, 2H), 4.89 (s, 5H), 4.42 (s, 4H), 3.79 (s, 2H), 2.88 (s, 2H), 1.99 (d, J=2.4 Hz, 6H); MS: m/z=502.0 [M+H]⁺

### Biological Testing Methods

### Biological Example 1: METTL3/14 Complex Recombinant Protein Production

Recombinant full-length human METTL3/14 complex was purchased from Active Motif (Carlsbad, CA) or produced by co-expression of full-length human METTL3 (UniProt ID: Q86U44-1, aa 1-580) and amino-terminal 6× His-tagged full-length METTL14 (UniProt ID: Q9HCE5-1, aa 1-456) in High Five insect cells using baculovirus infection. The METTL3/14 complex was purified from the High Five cells using two rounds of Nickel column purification followed by size-exclusion chromatography using a Superdex™ 200 column. The final protein was concentrated using ultra-filtration tubes at a molecular cutoff of 30 kilodaltons and stored frozen in protein buffer (25 mM HEPES, pH 7.5, 300 mM NaCl, 5% glycerol, 0.2 mM TCEP, 0.04% TritonX-100).

### Biological Example 2: Inhibition of METTL3/14 Enzymatic Activity—TR-FRET Assay

Compound IC50 values for the inhibition of METTL3/14 enzymatic activity were determined using a biochemical TR-FRET-based assay. Test compounds or controls (sinefungin and DMSO) diluted in 20% DMSO were incubated with 20-25 nM METTL3/14 complex in a microtiter plate in assay buffer (20 mM HEPES pH 7.5, 1 mM DTT, 0.4 U/μL RNAseOUT, and 0.01% Triton-X-100) for 30 minutes at 30° C. Final concentrations of DMSO and sinefungin were 1.2% and 1 mM, respectively. Following the incubation, a 5′-Cy5-labeled single-stranded RNA substrate (5′-Cy5-GCGGACUCCAGAUG-3′) and the co-substrate S-adenosylmethionine were diluted in assay buffer and added to each well for final concentrations of 50 nM and 300 nM, respectively. The reaction was incubated at 30° C. for 30 minutes before the addition of a rabbit anti-m6A antibody diluted in 4× stop buffer (200 mM HEPES pH 7.5, 600 mM NaCl, 0.4% BSA, and 400 mM KF) to a final concentration of 25 nM. The reaction was incubated at room temperature for 30 minutes followed by the addition of LANCE Europium-labeled anti-rabbit antibody diluted in 4× stop buffer to a final concentration of 2 nM. Following incubation of the plate at room temperature for 30 minutes, the emission values at 615 nm and 665 nm were read on a plate reader. Data were calculated as the ratio of the emission value at 665 nm (acceptor) and the emission value at 615 nm (donor). Percent inhibition of enzymatic activity by test compounds was calculated by normalizing the data to the 1% DMSO and 1 mM sinefungin control values, which represent 0% and 100% inhibition of enzymatic activity, respectively. Concentration-response-curves and IC50 values were generated using Collaborative Drug Discovery software.

### Biological Example 3: Inhibition of METTL3/14 Enzymatic Activity—MazF-FRET Biochemical Assay

Compound IC50 values for the inhibition of METTL3/14 enzymatic activity were determined in an orthogonal biochemical FRET-based assay using the methylation sensitive endoribonuclease, MazF. This assay uses a single-stranded RNA/DNA chimeric molecule containing a methylation sequence and labeled on the 5′ end with a carboxyfluorescein fluorophore (FAM) and on the 3′ end with a Black Hole Quencher 1 (BHQ1) dye (5′-FAM-d(CAT)GGACAd(TATGT)-BHQ1-3′). Methylation of the chimeric RNA/DNA molecule prevents cleavage by MazF; inhibition of methylation by test compounds allows MazF-dependent cleavage of the substrate and induction of fluorescence. Test compounds or sinefungin diluted in 20% DMSO/20 mM HEPES, pH 7.5 (or DMSO control) were incubated at 30° C. for 30 minutes with 20-25 nM METTL3/14 complex in a microtiter plate in assay buffer (20 mM HEPES pH 7.5, 5 mM KCl, 0.05% Tween-20, 1 mM DTT and 0.3 U/μL RNasin). Final concentrations of DMSO and sinefungin were 1% and 1 mM, respectively. Following the incubation, the FAM/BHQ1-labeled single-stranded RNA/DNA chimeric substrate and the co-substrate S-adenosylmethionine were diluted in assay buffer and added to the wells for final concentrations of 50 nM and 300 nM, respectively. The reaction was incubated at 30° C. for 60 minutes before the addition of the MazF enzyme diluted in 5× MazF Buffer (Takara Bio, cat. no. 2415A) for a final concentration of 0.2 U/μL in 0.95× MazF Buffer. Following an incubation at 30° C. for 60 min the fluorescence was read using a plate reader and standard FITC excitation and emission filters. Percent inhibition of enzymatic activity by test compounds was calculated by normalizing the data to the 1% DMSO and 1 mM sinefungin control values which represent 0% and 100% inhibition of enzymatic activity, respectively. Concentration-response-curves and IC50 values were generated using Collaborative Drug Discovery software.

### Biological Example 4: Viability Assay in MOLM-13 Cells

Effects of test compounds on the proliferation and viability of human AML cells was evaluated using the MOLM-13 cell line (AddexBio Technologies cat. no. C0003003). MOLM-13 cells were plated in tissue-culture treated microtiter plates at a final concentration of 50,000 cells/ml in complete media (RPMI-1640 with addition of fetal bovine serum to 10%). Test compounds or controls (Bortezomib or DMSO) diluted in 2% DMSO/media were added to each well for final concentrations of 0.2% DMSO and 0.1-1 μM Bortezomib. The cells were incubated for 72 hours at 37° C. and 5% CO₂. At the end of the 72 hour incubation, plates were removed from the incubator, cooled to room temperature and equal volume of CellTiter-Glo 2.0 (Promega) added to each well. After mixing and incubating the plate at room temperature for 10 minutes the luminescence was read using a plate reader. Percent inhibition of cell viability by test compounds was calculated by normalizing the data to the 0.2% DMSO (0% loss of viability) and 0.1-1 μM Bortezomib (100% loss of viability) control values. Concentration-response-curves and IC50 values were generated using Collaborative Drug Discovery software.

### Results

Results of certain compounds are shown in Table 1. Table legend: TR-FRET/MazF-FRET: A is IC50<100 nM; B is IC50 100-1000 nM; C is IC50>1000 nM. MOLM13: * is >10 μM; **is 1-10 μM; *** is <1 μM.

TABLE 1

| Compound No. | TR-FRET | MazF-FRET | MOLM-13 |
|---|---|---|---|
| 1 | C | C | |
| 2 | C | | |
| 3 | C | C | * |
| 4 | C | | |
| 5 | B | B | |
| 6 | C | | |
| 7 | C | C | * |
| 8 | C | | |
| 9 | C | C | * |
| 10 | C | C | * |
| 11 | C | C | * |
| 12 | C | C | |
| 13 | B | C | * |
| 14 | C | C | |
| 15 | C | C | * |
| 16 | C | | |
| 17 | C | C | * |
| 18 | C | | |
| 19 | C | | |
| 20 | C | | |
| 21 | C | C | |
| 22 | C | | |
| 23 | C | C | |
| 24 | C | B | |
| 25 | C | | |
| 26 | C | C | * |
| 27 | C | | |
| 28 | B | C | * |
| 29 | C | C | * |
| 30 | C | C | * |
| 31 | C | C | |
| 32 | C | C | |
| 33 | C | C | |
| 34 | C | C | |
| 35 | C | B | |
| 36 | | | |
| 37 | C | | |
| 38 | B | C | * |
| 39 | | C | |
| 40 | C | | |
| 41 | C | | |
| 42 | C | C | |
| 43 | C | C | |
| 44 | C | | |
| 45 | C | C | |
| 46 | C | C | * |
| 47 | C | | |
| 48 | C | C | |
| 49 | C | C | ** |
| 50 | C | C | |
| 51 | C | C | |
| 52 | C | | |
| 53 | C | | |
| 54 | C | | |
| 55 | C | | |
| 56 | C | | |
| 57 | C | | |
| 58 | C | | |
| 59 | C | | |
| 60 | C | | |
| 61 | C | | |
| 62 | C | | |
| 63 | C | | |
| 64 | C | | |
| 65 | C | | |
| 66 | C | | |
| 67 | C | | |
| 68 | C | C | |
| 69 | C | C | * |
| 70 | B | B | * |
| 71 | B | C | * |
| 72 | B | B | ** |
| 73 | B | B | ** |
| 74 | B | B | * |
| 75 | B | B | * |
| 76 | A | B | ** |
| 77 | A | B | ** |
| 78 | C | C | ** |
| 79 | A | A | ** |
| 80 | B | C | ** |
| 81 | C | C | ** |
| 82 | C | C | ** |
| 83 | A | A | ** |
| 84 | B | B | ** |
| 85 | B | B | ** |
| 86 | C | B | ** |
| 87 | A | B | ** |
| 88 | A | A | ** |
| 89 | A | A | ** |
| 90 | B | B | |
| 91 | C | | |
| 92 | C | C | |
| 93 | C | B | ** |
| 94 | C | C | ** |
| 95 | C | C | |
| 96 | C | C | ** |
| 97 | C | C | |
| 98 | C | | |
| 99 | C | C | * |
| 100 | C | C | ** |
| 101 | C | B | ** |
| 102 | B | B | ** |
| 103 | C | C | |
| 104 | C | | |
| 105 | C | C | ** |
| 106 | C | C | |
| 107 | C | | |
| 108 | C | C | * |
| 109 | C | C | |
| 110 | A | | ** |
| 111 | A | | * |
| 112 | A | | ** |
| 113 | A | | ** |
| 114 | A | | ** |
| 115 | A | | * |
| 116 | A | | * |
| 117 | A | | * |
| 118 | A | | ** |
| 119 | A | | ** |
| 120 | B | | |
| 121 | A | | |
| 122 | B | | |
| 123 | A | | * |
| 124 | A | | * |
| 125 | A | | * |
| 126 | A | | * |
| 127 | A | | ** |
| 128 | A | | ** |
| 129 | A | | ** |
| 130 | A | | ** |
| 131 | B | | |
| 132 | A | | ** |
| 133 | B | | |
| 134 | A | | * |
| 135 | A | | ** |
| 136 | A | | ** |
| 137 | A | | ** |
| 138 | A | | ** |
| 139 | B | | |
| 140 | A | | ** |
| 141 | A | | * |
| 142 | A | | ** |
| 143 | A | | ** |
| 144 | A | | ** |
| 145 | A | | ** |
| 146 | A | | ** |
| 147 | A | | ** |
| 148 | A | | ** |
| 149 | A | | ** |
| 150 | A | | ** |
| 151 | A | | *** |
| 152 | A | | *** |
| 153 | A | | *** |
| 154 | A | | ** |
| 155 | A | | ** |
| 156 | A | | * |
| 157 | A | | ** |

### EQUIVALENTS

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

What is claimed is:

1. A compound of Formula (I′): 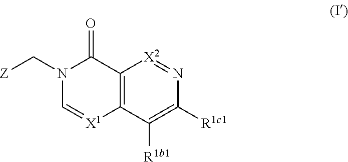
or a pharmaceutically acceptable salt thereof, wherein:
X¹ is CR^{1a} or N;
R^{1a} is selected from the group consisting of H, C₁₋₆alkyl, halo, cycloalkyl, aryl, and heteroaryl; wherein the C₁₋₆alkyl of R^{1a} is unsubstituted or substituted with aryl or heteroaryl, wherein the aryl and heteroaryl is unsubstituted or substituted with one or more substituents selected from the group consisting of of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NR^{1e}C(O)R^{1e};
X² is CH or N;
R^{1b1} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b1} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, —CH(N(R^{1e})₂)COOR^{1e} and —NR^{1e}C(O)R^{1e};
wherein the C₁₋₆alkyl and alkynyl of R^{1b1} is unsubstituted or substituted with one or more substituents selected from the group consisting of halo, aryl, heteroaryl, heterocyclyl, and cycloalkyl;
R^{1c1} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3; 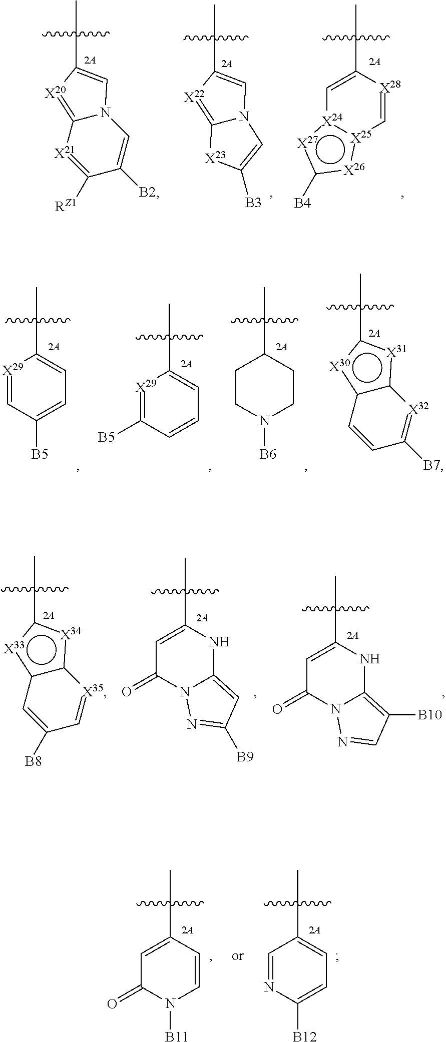
X²⁰ is CR^{20a} or N; wherein R^{20a} is H, halo, CN, aryl, or heteroaryl;
X²¹ is CH or N;
X²² is CR^{22a} or N; wherein R^{22a} is H, halo, CN, aryl, or heteroaryl;
X²³ is CH₂, S, O, NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
X²⁹ is CH or N;
X³⁰ and X³¹ are independently CH, O, S, N, or NH;
X³² is CH or N;
X³³ and X³⁴ are independently CH, O, S, N, or NH;
X³⁵ is CH or N;
R^{Z1} is H, halo, —CN, or —S(O)₂F;
B2, B3, B4, B7, B8, B9, and B10 are independently selected from the group consisting of H, C₁₋₆alkyl, CN, —S(O)₂F, —CH₂—NR^{2a}R^{2b}, 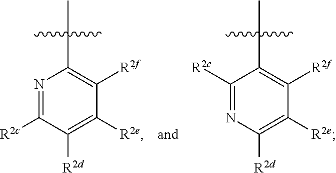
R^{2a} is H or C₁₋₆alkyl;
R^{2b} is selected from the group consisting of H, C₁₋₆alkyl, aryl, heteroaryl, heterocyclyl, —C(NH)-aryl, —S(O)(NH)R^{2g}, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, —S(O)₂F, —S(O(NH)R^{2g}, alkoxy, aryl, haloaryl, cycloalkyl, halocycloalkyl, hydroxycycloalkyl, and heterocyclyl, wherein the cycloalkyl is unsubstituted or substituted with one or more substituents selected from the group consisting of C₁₋₆alkyl, —(CH₂)ₙF, —(CH₂)ₙBr, or —(CH₂)ₙCN and wherein the heterocyclyl is unsubstituted or substituted with C₁₋₆alkyl; or
R^{2a} and R^{2b} combine to form a 5-7-membered heterocyclyl, wherein the heterocyclyl is unsubstituted or substituted with one or more substituents selected from the group consisting of C₁₋₆alkyl and cycloalkyl;
R^{2c}, R^{2d}, R^{2e}, and R^{2f} are each independently H, C₁₋₆alkyl, heterocyclyl, or —NHR^{2g;}
R^{2g} is selected from the group consisting of H, C₁₋₆alkyl, and cycloalkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;
B5, B6, B11, and B12 are independently 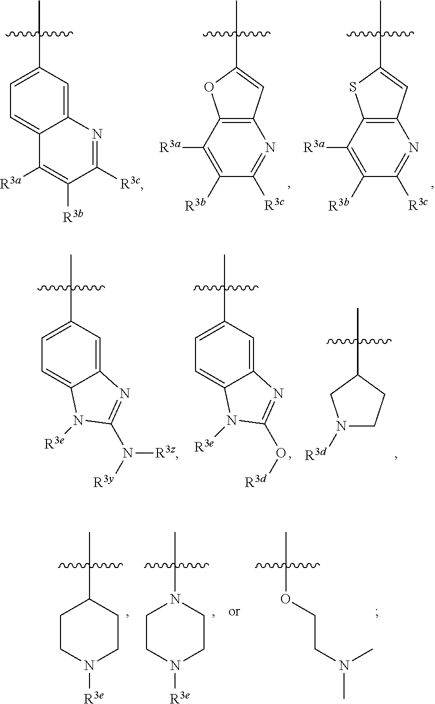
R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, —OR^{3x′}, and —CO₂R^{x};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

2. A compound of Formula (I): 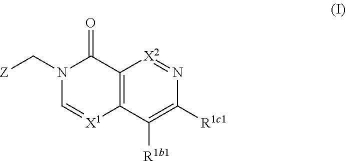
or a pharmaceutically acceptable salt thereof, wherein:
X¹ is CR^{1a} or N;
R^{1a} is selected from the group consisting of H, C₁₋₆alkyl, halo, cycloalkyl, and heteroaryl;
wherein the C₁₋₆alkyl of R^{1a} is unsubstituted or substituted with aryl;
X² is CH or N;
R^{1b1} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b1} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b1} is unsubstituted or substituted with one or more substituents selected from the group consisting of aryl, heterocyclyl, and cycloalkyl;
R^{1c1} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
Z is 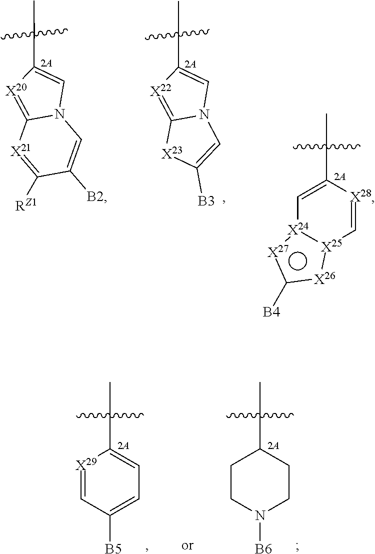
X²⁰ is CR^{20a} or N; wherein R^{20a} is H, halo, CN, aryl, or heteroaryl;
X²¹ is CH or N;
X²² is CR^{22a} or N; wherein R^{22a} is H, halo, CN, aryl, or heteroaryl;
X²³ is CH₂, S, O, NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
X²⁹ is CH or N;
B2, B3, and B4 are independently selected from the group consisting of H, C₁₋₆alkyl, CN, —CH₂—NR^{2a}R^{2b}, 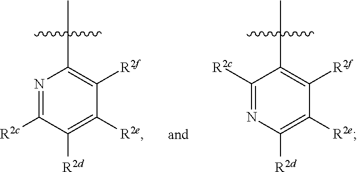
R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;
B5 and B6 are independently 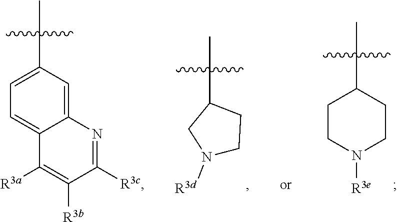
R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of Hand C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

3. A compound of Formula (II), (III), (IV), (V), or (VI): 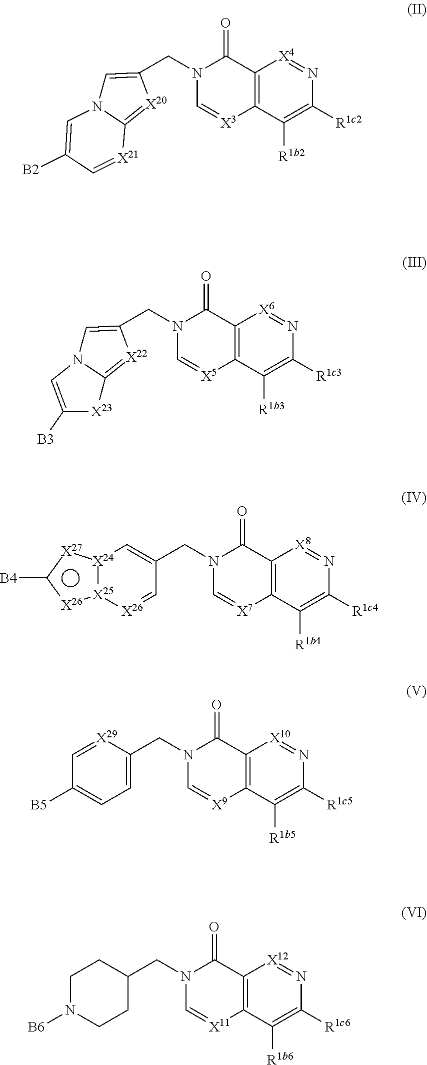
or a pharmaceutically acceptable salt thereof, wherein:
X³, X⁵, X⁷, X⁹, and X¹¹ are independently CR^{1a} or N;
R^{1a} is selected from the group consisting of H, C₁₋₆alkyl, halo, cycloalkyl, and heteroaryl;
wherein the C₁₋₆alkyl of R^{1a} is unsubstituted or substituted with aryl;
X⁴, X⁶, X⁸, X¹⁰, and X¹² are independently CH or N;
R^{1b2}, R^{1b3}, R^{1b4}, R^{1b5}, and R^{1b6} are independently selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b2}, R^{1b3}, R^{1b4}, R^{1b5}, and R^{1b6} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b2}, R^{1b3}, R^{1b4}, R^{1b5}, and R^{1b6} is unsubstituted or substituted with one or more substituents selected from the group consisting of aryl, heterocyclyl, and cycloalkyl;
R^{1c2}, R^{1c3}, and R^{1c4} are independently selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁰ is CR^{20a} or N; wherein R^{20a} is H, halo, CN, aryl, or heteroaryl;
X²¹ is CH or N;
X²² is CR^{22a} or N; wherein R^{22a} is H, halo, aryl, or heteroaryl;
X²³ is CH, S, O, or NH;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
B2, B3, and B4 are independently selected from the group consisting of H, C₁₋₆alkyl, CN, —CH₂—NR^{2a}R^{2b}, 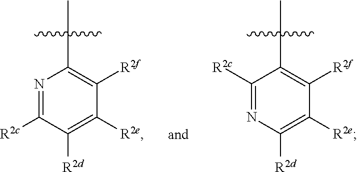
R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6;
X²⁹ is CH or N;
B5 and B6 are independently 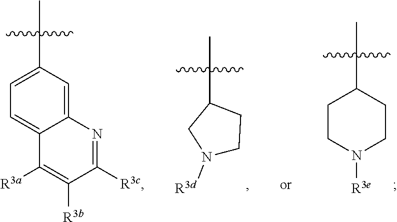
R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted cycloalkyl; and
R^{3e} is selected from the group consisting of H and C₁₋₆alkyl; wherein C₁₋₆alkyl is unsubstituted or substituted cycloalkyl.

4. A compound of Formula (II): 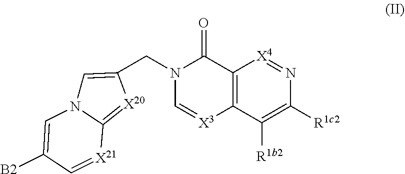
or a pharmaceutically acceptable salt thereof, wherein:
X³ is CR^{1a} or N;
R^{1a} is selected from the group consisting of H, C₁₋₆alkyl, halo, cycloalkyl, and heteroaryl;
wherein the C₁₋₆alkyl of R^{1a} is unsubstituted or substituted with aryl;
X⁴ is CH or N;
R^{1b2} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b2} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b2} is unsubstituted or substituted with one or more substituents selected from the group consisting of aryl, heterocyclyl, and cycloalkyl;
R^{1c2} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁰ is CR^{20a} or N; wherein R^{20a} is H, halo, CN, aryl, or heteroaryl;
X²¹ is CH or N;
B2 is selected from the group consisting of H, C₁₋₆alkyl, CN, —CH₂—NR^{2a}R^{2b}, 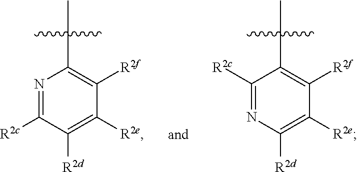
R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

5. The compound of claim 4, or a pharmaceutically acceptable salt thereof, wherein the compound is of Formula (IIa):

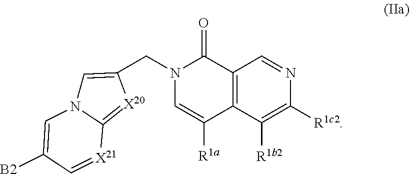

6. The compound of claim 4 or 5, or a pharmaceutically acceptable salt thereof, wherein the compound is of Formula (IIb):

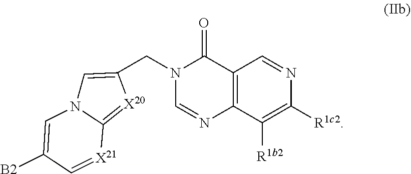

7. The compound of any one of claims 4, 5, and 6, or a pharmaceutically acceptable salt thereof, wherein the compound is of Formula (IIc):

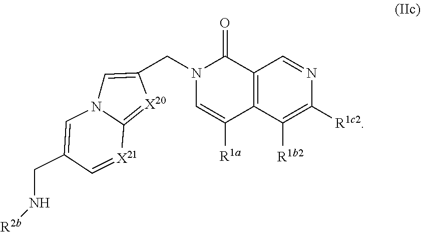

8. A compound of Formula (III): 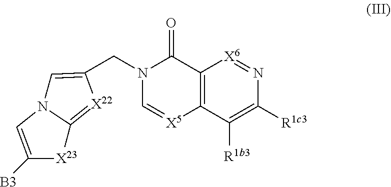
or a pharmaceutically acceptable salt thereof, wherein:
X⁵ is CR^{1a} or N;
R^{1a} is selected from the group consisting of H, C₁₋₆alkyl, halo, cycloalkyl, and heteroaryl;
wherein the C₁₋₆alkyl of R^{1a} is unsubstituted or substituted with aryl;
X⁶ is CH or N;
R^{1b3} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b3} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b3} is unsubstituted or substituted with one or more substituents selected from the group consisting of aryl, heterocyclyl, and cycloalkyl;
R^{1c3} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²² is CR^{22a} or N; wherein R^{22a} is H, halo, aryl, or heteroaryl;
X²³ is CH, S, O, or NH;
B3 is selected from the group consisting of H, C₁₋₆alkyl, CN, —CH₂—NR^{2a}R^{2b}, 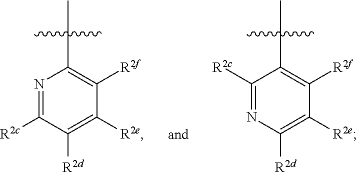
R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

9. The compound of claim 8, or a pharmaceutically acceptable salt thereof, wherein the compound is of Formula (IIIa):

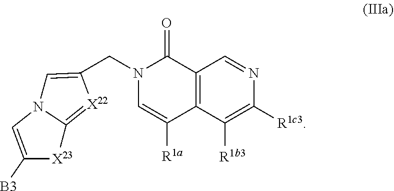

10. The compound of claim 8 or 9, or a pharmaceutically acceptable salt thereof, wherein the compound is of Formula (IIIb):

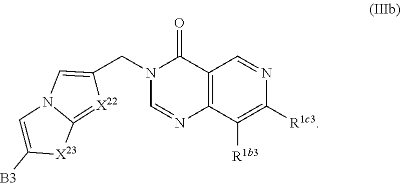

11. The compound of any one of claims 8, 9, and 10, or a pharmaceutically acceptable salt thereof, wherein the compound is of Formula (IIIc):

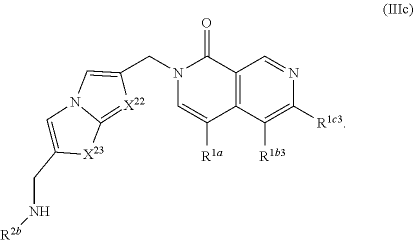

12. A compound of Formula (IV): 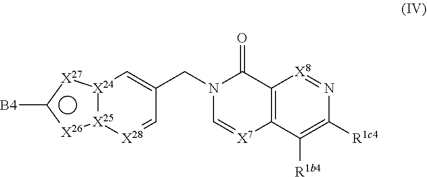
or a pharmaceutical y acceptable salt thereof, wherein:
X⁷ is CR^{1a} or N;
R^{1a} is selected from the group consisting of H, C₁₋₆alkyl, halo, cycloalkyl, and heteroaryl;
wherein the C₁₋₆alkyl of R^{1a} is unsubstituted or substituted with aryl;
X⁸ is CH or N;
R^{1b4} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b4} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b4} is unsubstituted or substituted with one or more substituents selected from the group consisting of aryl, heterocyclyl, and cycloalkyl;
R^{1c4} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁴ and X²⁵ are independently C or N;
X²⁶ and X²⁷ are independently CH, O, S, N, or NR^{26a}; wherein R^{26a} is H or C₁₋₆alkyl;
X²⁸ is CH or N;
B4 is selected from the group consisting of H, C₁₋₆alkyl, CN, —CH₂—NR^{2a}R^{2b}, 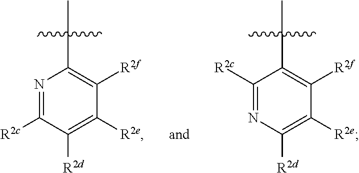
R^{2a} is H or C₁₋₆alkyl;
R^{2b} is selected from the group consisting of H, C₁₋₆alkyl, aryl, heteroaryl, heterocyclyl, —C(NH)-aryl, and cycloalkyl;
wherein the C₁₋₆alkyl, aryl, heteroaryl, heterocyclyl, and cycloalkyl of R^{2b} is unsubstituted or substituted with one or more substituents selected from the group consisting of C₁₋₆alkyl, OH, —N(R^{2h})₂, —C(NH)-aryl, alkoxy, aryl, haloaryl, cycloalkyl, halocycloalkyl, hydroxycycloalkyl, and heterocyclyl; or
R^{2a} and R^{2b} combine to form a 5-7-membered heterocyclyl, wherein the heterocyclyl is unsubstituted or substituted with C₁₋₆alkyl;
R^{2c}, R^{2d}, R^{2e}, and R^{2f} are each independently H, C₁₋₆alkyl, heterocyclyl, or —NHR^{2g};
R^{2g} is selected from the group consisting of H and C₁₋₆alkyl, wherein the C₁₋₆alkyl is unsubstituted or substituted with cycloalkyl;
each R^{2h} is independently H, C₁₋₆alkyl, —(CH₂)ₙCl, or —(CH₂)ₙOCH₃;
n is 1, 2, 3, 4, 5, or 6.

13. The compound of claim 12, or a pharmaceutically acceptable salt thereof, wherein the compound is of Formula (IVa):

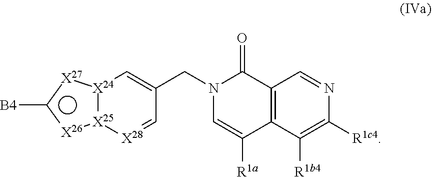

14. The compound of claim 12 or 13, or a pharmaceutically acceptable salt thereof, wherein the compound is of Formula (IVb):

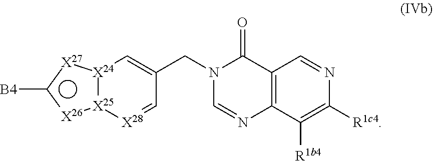

15. The compound of any one of claims 12, 13 or 14, or a pharmaceutically acceptable salt thereof, wherein the compound is of Formula IVc):

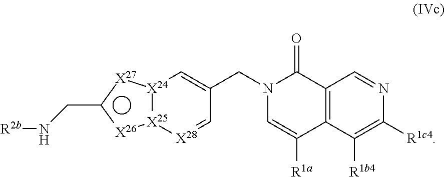

16. A compound of Formula (V): 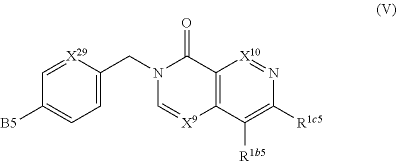
or a pharmaceutically acceptable salt thereof, wherein:
X⁹ is CR^{1a} or N;
R^{1a} is selected from the group consisting of H, C₁₋₆alkyl, halo, cycloalkyl, and heteroaryl;
wherein the C₁₋₆alkyl of R^{1a} is unsubstituted or substituted with aryl;
X¹⁰ is CH or N;
R^{1b}s is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b5} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b}s is unsubstituted or substituted with one or more substituents selected from the group consisting of aryl, heterocyclyl, and cycloalkyl;
R^{1e} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
X²⁹ is CH or N;
B5 is 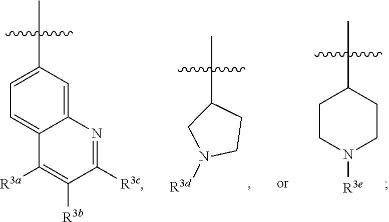
R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of H and C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

17. The compound of claim 16, or a pharmaceutically acceptable salt thereof, wherein the compound is of Formula (Va):

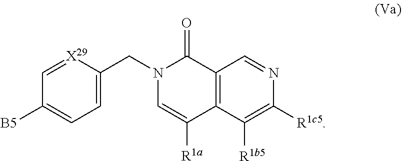

18. The compound of claim 16 or 17, or a pharmaceutically acceptable salt thereof, wherein the compound is of Formula (Vb):

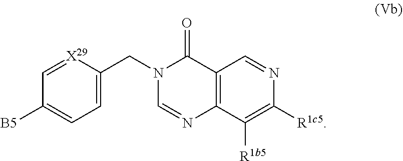

19. A compound of Formula (VI): 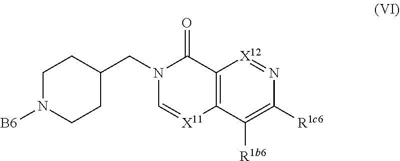
or a pharmaceutically acceptable salt thereof, wherein:
X¹¹ is CR^{1a} or N;
R^{1a} is selected from the group consisting of H, C₁₋₆alkyl, halo, cycloalkyl, and heteroaryl;
wherein the C₁₋₆alkyl of R^{1a} is unsubstituted or substituted with aryl;
X¹² is CH or N;
R^{1b6} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b6} is unsubstituted or substituted with one or more substituents selected from the group consisting of C₁₋₆alkyl, halo, —CN, —(CH₂)ₘNH₂, —(CH₂)ₘOH, —C(O)R^{1e}, —C(O)N(R^{1e})₂, —(CH₂)ₘCOOH, aryl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b6} is unsubstituted or substituted with one or more substituents selected from the group consisting of aryl, heterocyclyl, and cycloalkyl;
R^{1c6} is selected from the group consisting of H, C₁₋₆alkyl, halo, —CN, —N(R^{1d})₂, and —OR^{1d};
wherein R^{1d} is H, C₁₋₆alkyl, —(CH₂)ₘCN, —(CH₂)ₘNH₂, cycloalkyl, or heterocyclyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
B6 is 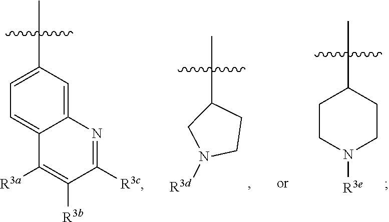
R^{3a}, R^{3b}, and R^{3c} are each independently H, halogen, C₁₋₆alkyl, —OR^{3x}, —NR^{3y}R^{3z}, cycloalkyl, or heterocyclyl; wherein heterocyclyl is unsubstituted or substituted with one or more substituents selected from the group consisting of C₁₋₆alkyl, halogen, —NR^{3y′}R^{3z′}, and —OR^{3x′};
R^{3x} and R^{3x′} are each independently selected from the group consisting of H and C₁₋₆alkyl;
R^{3y}, R^{3z}, R^{3y′}, and R^{3z′} are each independently H, C₁₋₆alkyl, or cycloalkyl; wherein C₁₋₆alkyl is unsubstituted or substituted with one or more substituents selected from the group consisting of cycloalkyl and —OCH₃;
R^{3d} is selected from the group consisting of Hand C₁₋₆alkyl; wherein C₁₋₆alkyl is unsubstituted or substituted with cycloalkyl; and
R^{3e} is selected from the group consisting of H and C₁₋₆alkyl; wherein C₁₋₆alkyl is unsubstituted or substituted with cycloalkyl.

20. The compound of claim 19, or a pharmaceutically acceptable salt thereof, wherein the compound is of Formula (VIa):

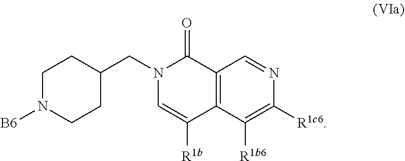

21. The compound of claim 19 or 20, or a pharmaceutically acceptable salt thereof, wherein the compound is of Formula (VIb):

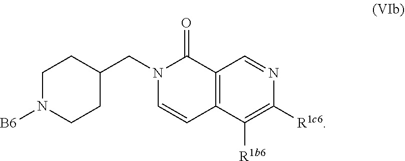

22. The compound of any one of claims 1-4, 8, 12, 16, and 19, or a pharmaceutically acceptable salt thereof, wherein X¹, X³, X⁵, X⁷, X⁹, or X¹¹ is CR^{1a}.

23. The compound of any one of claims 1-4, 6, 12, 16, 19, and 22, or a pharmaceutically acceptable salt thereof, wherein R^{1a} is H.

24. The compound of any one of claims 1-4, 6, 12, 16, 19, and 22, or a pharmaceutically acceptable salt thereof, wherein R^{1a} is C₁₋₆alkyl, wherein the C₁₋₆alkyl is unsubstituted or substituted with aryl.

25. The compound of any one of claims 1-4,6, 12, 16, 19, and 22, or a pharmaceutically acceptable salt thereof, wherein R^{1a} is cycloalkyl or heteroaryl.

26. The compound of any one of claims 1-4, 6, 12, 16, 19, and 22, or a pharmaceutically acceptable salt thereof, wherein R^{1a} is halo.

27. The compound of any one of claims 1-4, 8, 12, 16, and 19, or a pharmaceutically acceptable salt thereof, wherein X¹, X³, X⁵, X⁷, X⁹, or X¹¹ is N.

28. The compound of any one of claims 1-4, 6, 12, 16, 19, and 22-27, or a pharmaceutically acceptable salt thereof, wherein X², X⁴, X⁶, X⁸, X¹⁰, or X¹² is CH.

29. The compound of any one of claims 1-4, 6, 12, 16, 19, and 22-27, or a pharmaceutically acceptable salt thereof, wherein X², X⁴, X⁶, X⁸, X¹⁰, or X¹² is N.

30. The compound of any one of claims 1-29, or a pharmaceutically acceptable salt thereof, wherein R^{1c1}, R^{1c2}, R^{1c3}, R^{1c4}, R^{1c5}, or R^{1c6} is H.

31. The compound of any one of claims 1-29, or a pharmaceutically acceptable salt thereof, wherein R^{1c1}, R^{1c2}, R^{1c3}, R^{1C4}, R^{1c5}, or R^{1c6} is C₁₋₆alkyl.

32. The compound of any one of claims 1-29, or a pharmaceutically acceptable salt thereof, wherein R^{1c1}, R^{1c2}, R^{1c3}, R^{1c4}, R^{1c5}, or R^{1c6} is halo, —CN, —N(R^{1d})₂, or —OR^{1d}.

33. The compound of any one of claims 1-29 and 32, or a pharmaceutically acceptable salt thereof, wherein R^{1d} is H or C₁₋₆alkyl.

34. The compound of any one of claims 1-29 and 32, or a pharmaceutically acceptable salt thereof, wherein R^{1d} is —(CH₂)ₘCN or —(CH₂)ₘNH₂.

35. The compound of any one of claims 1-29 and 32, or a pharmaceutically acceptable salt thereof, wherein R^{1d} is cycloalkyl or heterocyclyl.

36. The compound of any one of claims 1-35, or a pharmaceutically acceptable salt thereof, wherein R^{1b1}, R^{1b2}, R^{1b3}, R^{1b4}, R^{1b5}, or R^{1b6} is aryl, wherein the aryl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}.

37. The compound of any one of claims 1-35, or a pharmaceutically acceptable salt thereof, wherein R^{1b1}, R^{1b2}, R^{1b3}, R^{1b4}, R^{1b5}, or R^{1b6} is heteroaryl or heterocyclyl, wherein the heteroaryl and heterocyclyl of R^{1b} is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}.

38. The compound of any one of claims 1-35, or a pharmaceutically acceptable salt thereof, wherein R^{1b1}, R^{1b2}, R^{1b3}, R^{1b4}, R^{1b5}, or R^{1b6} is cycloalkyl or cycloalkenyl, wherein the cycloalkyl and cycloalkenyl is unsubstituted or substituted with one or more substituents selected from the group consisting of C₁₋₆alkyl, halo, —CN, —(CH₂)ₙOH, —C(O)R^{1e}, —C(O)N(R^{1e})₂, —(CH₂)ₙCOOH, aryl, —(CH₂)ₙaryl, cycloalkyl, oxo, —OR^{1e}, —N(R^{1e})₂, —S(O)₂R^{1e}, —S(O)₂N(R^{1e})₂, —NHS(O)₂R^{1e}, and —NHC(O)R^{1e}.

39. The compound of any one of claims 36-38, or a pharmaceutically acceptable salt thereof, wherein the aryl, cycloalkyl, cycloalkenyl, heteroaryl, and heterocyclyl is unsubstituted.

40. The compound of any one of claims 1-35, or a pharmaceutically acceptable salt thereof, wherein R^{1b1}, R^{1b2}, R^{1b3}, R^{1b4}, R^{1b5}, or R^{1b6} is C₁₋₆alkyl or alkynyl, wherein the C₁₋₆alkyl and alkynyl is unsubstituted or substituted with one or more substituents selected from the group consisting of aryl, heterocyclyl, and cycloalkyl.

41. The compound of any one of claims 1-35, or a pharmaceutically acceptable salt thereof, wherein R^{1b1}, R^{1b2}, R^{1b3}, R^{1b4}, R^{1b5}, or R^{1b6} is H.

42. The compound of any one of claims 1-35, or a pharmaceutically acceptable salt thereof, wherein R^{1b1}, R^{1b2}, R^{1b3}, R^{1b4}, R^{1b5}, or R^{1b6} is halo.

43. The compound of any one of claims 1-35, or a pharmaceutically acceptable salt thereof, wherein R^{1b1}, R^{1b2}, R^{1b3}, R^{1b4}, R^{1b5}, or R^{1b6} is —OR^{1e} or —N(R^{1e})₂.

44. The compound of any one of claims 1-7 and 22-43, or a pharmaceutically acceptable salt thereof, wherein X²⁰ is CR^{20a}.

45. The compound of any one of claims 1-7 and 22-44, or a pharmaceutically acceptable salt thereof, wherein R^{20a} is H.

46. The compound of any one of claims 1-7 and 22-44, or a pharmaceutically acceptable salt thereof, wherein R^{20a} is halo, CN, aryl, or heteroaryl.

47. The compound of any one of claims 1-7 and 22-43, or a pharmaceutically acceptable salt thereof, wherein X²⁰ is N.

48. The compound of any one of claims 1-7 and 22-47, or a pharmaceutically acceptable salt thereof, wherein X²¹ is CH.

49. The compound of any one of claims 1-7 and 22-47, or a pharmaceutically acceptable salt thereof, wherein X²¹ is N.

50. The compound of any one of claims 1-3, 8-11, and 22-43, or a pharmaceutically acceptable salt thereof, wherein X²² is CR^{22a}.

51. The compound of any one of claims 1-3, 8-11, 22-43, and 50, or a pharmaceutically acceptable salt thereof, wherein R^{22a} is H.

52. The compound of any one of claims 1-3, 8-11, 22-43, and 50, or a pharmaceutically acceptable salt thereof, wherein R^{22a} is halo or aryl.

53. The compound of any one of claims 1-3, 8-11, and 22-43, or a pharmaceutically acceptable salt thereof, wherein X²² is N.

54. The compound of any one of claims 1-3, 8-11, 22-43, and 50-53, or a pharmaceutically acceptable salt thereof, wherein X²³ is CH₂.

55. The compound of any one of claims 1-3, 8-11, 22-43, and 50-53, or a pharmaceutically acceptable salt thereof, wherein X²³ is NH.

56. The compound of any one of claims 1-3, 8-11, 22-43, and 50-53, or a pharmaceutically acceptable salt thereof, wherein X²³ is S or O.

57. The compound of any one of claims 1-3, 12-15, and 22-43, or a pharmaceutically acceptable salt thereof, wherein X²⁴ and X²⁵ are C.

58. The compound of any one of claims 1-3, 12-15, and 22-43, or a pharmaceutically acceptable salt thereof, wherein X²⁴ is N and X²⁵ is C.

59. The compound of any one of claims 1-3, 12-15, 22-43, and 57-58, or a pharmaceutically acceptable salt thereof, wherein X²⁶ and X²⁷ are independently N or NR^{26a}; wherein R^{26a} is H or C₁₋₆alkyl.

60. The compound of any one of claims 1-3, 12-15, 22-43, and 57-58, or a pharmaceutically acceptable salt thereof, wherein X²⁶ and X²⁷ are CH.

61. The compound of any one of claims 1-3, 12-15, 22-43, and 57-60, or a pharmaceutically acceptable salt thereof, wherein X²⁸ is CH.

62. The compound of any one of claims 1-3, 12-15, 22-43, and 57-60, or a pharmaceutically acceptable salt thereof, wherein X²⁸ is N.

63. The compound of any one of claims 1-6, 8-10, 12-14, and 22-62, or a pharmaceutically acceptable salt thereof, wherein B2, B3, or B4 is —CH₂—NR^{2a}R^{2b}.

64. The compound of any one of claims 1-15 and 22-63, or a pharmaceutically acceptable salt thereof, wherein R^{2b} is C₁₋₆alkyl, wherein the C₁₋₆alkyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl.

65. The compound of any one of claims 1-15 and 22-63, or a pharmaceutically acceptable salt thereof, wherein R^{2b} is cycloalkyl; wherein the cycloalkyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl.

66. The compound of any one of claims 1-15 and 22-63, or a pharmaceutically acceptable salt thereof, wherein R^{2b} is selected from the group consisting of heteroaryl and heterocyclyl; wherein the heteroaryl and heterocyclyl is unsubstituted or substituted with one or more substituents selected from the group consisting of C₁₋₆alkyl, OH, alkoxy, aryl, haloaryl, cycloalkyl, halocycloalkyl, and heterocyclyl; wherein the heterocyclyl is unsubstituted or substituted with C₁₋₆alkyl.

67. The compound of any one of claims 1-6, 8-10, 12-14, and 22-66, or a pharmaceutically acceptable salt thereof, wherein R^{2a} is H.

68. The compound of any one of claims 1-6, 8-10, 12-14, and 22-63, or a pharmaceutically acceptable salt thereof, wherein R^{2a} and R^{2b} combine to form a 5-7-membered heterocyclyl, wherein the heterocyclyl is substituted with C₁₋₆alkyl.

69. The compound of anyone of claims 1-6, 8-10, 12-14, and 22-63, or a pharmaceutically acceptable salt thereof, wherein R^{2a} and R^{2b} combine to form a 5-7-membered heterocyclyl, wherein the heterocyclyl is unsubstituted.

70. The compound of any one of claims 1-6, 8-10, 12-14, and 22-62, or a pharmaceutically acceptable salt thereof, wherein B2, B3, or B4 is

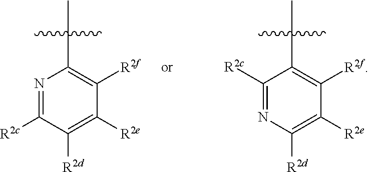

71. The compound of any one of claims 1-6, 8-10, 12-14, 22-62, and 70, or a pharmaceutically acceptable salt thereof, wherein one of R^{2c}, R^{2d}, R^{2e}, and R^{2f} is —NHR^{2g} and the others are H.

72. The compound of any one of claims 1-6, 8-10, 12-14, 22-62, and 70-71, or a pharmaceutically acceptable salt thereof, wherein R^{2g} is C₁₋₆alkyl, wherein the C₁₋₆alkyl is unsubstituted or substituted with cycloalkyl.

73. The compound of any one of claims 1-6, 8-10, 12-14, 22-62, and 70-71, or a pharmaceutically acceptable salt thereof, wherein R^{2g} is H.

74. The compound of any one of claims 1-6, 8-10, 12-14, 22-62, and 70, or a pharmaceutically acceptable salt thereof, wherein one of R^{2c}, R^{2d}, R^{2e}, and R^{2f} is C₁₋₆alkyl or heterocyclyl and the others are H.

75. The compound of any one of claims 1-6, 8-10, 12-14, and 22-62, or a pharmaceutically acceptable salt thereof, wherein B2, B3, or B4 is C₁₋₆alkyl.

76. The compound of any one of claims 1-6, 8-10, 12-14, and 22-62, or a pharmaceutically acceptable salt thereof, wherein B2, B3, or B4 is H or CN.

77. The compound of any one of claims 1-3, 16-18, and 22-43, or a pharmaceutically acceptable salt thereof, wherein X²⁹ is CH.

78. The compound of any one of claims 1-3, 16-18, and 22-43, or a pharmaceutically acceptable salt thereof, wherein X²⁹ is N.

79. The compound of any one of claims 1-3, 16-43, and 77-78, or a pharmaceutically acceptable salt thereof, wherein B5 or B6 is

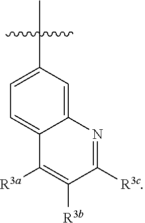

80. The compound of any one of claims 1-3, 16-43, and 77-79, or a pharmaceutically acceptable salt thereof, wherein one or two of R^{3a}, R^{3b}, and R^{3c} is —OR^{3x} and the others of R^{3a}, R^{3b}, and R^{3c} are H.

81. The compound of any one of claims 1-3, 16-43, and 77-80, or a pharmaceutically acceptable salt thereof, wherein R^{3x} is C₁₋₆alkyl.

82. The compound of any one of claims 1-3, 16-43, and 77-79, or a pharmaceutically acceptable salt thereof, wherein one or two of R^{3a}, R^{3b}, and R^{3c} is —NR^{3y}R^{3z} and the others of R³, R^{3b}, and R^{3c} are H.

83. The compound of any one of claims 1-3, 16-43, 77-79, and 82, or a pharmaceutically acceptable salt thereof, wherein one of R^{3y} and R^{3z} is C₁₋₆alkyl; wherein C₁₋₆alkyl is unsubstituted or substituted with one or more substituents selected from the group consisting of cycloalkyl and —OCH₃.

84. The compound of any one of claims 1-3, 16-43, and 77-79, or a pharmaceutically acceptable salt thereof, wherein one or two of R^{3a}, R^{3b}, and R^{3c} is heterocyclyl wherein the heterocyclyl is unsubstituted or substituted with one or more substituents selected from the group consisting of C₁₋₆alkyl, halogen, and —OH, and the others of R^{3a}, R^{3b}, and R^{3c} are H.

85. The compound of any one of claims 1-3, 16-43, and 77-78, or a pharmaceutically acceptable salt thereof, wherein B5 or B6 is

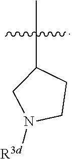

86. The compound of any one of claims 1-3, 16-43, 77-78, and 85, or a pharmaceutically acceptable salt thereof, wherein R^{3d} is C₁₋₆alkyl; wherein C₁₋₆alkyl is unsubstituted or substituted with cycloalkyl.

87. The compound of any one of claims 1-3, 16-43, and 77-78, or a pharmaceutically acceptable salt thereof, wherein B5 or B6 is

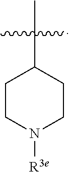

88. The compound of any one of claims 1-3, 16-43, 77-78, and 87, or a pharmaceutically acceptable salt thereof, wherein R^{3e} is C₁₋₆alkyl; wherein C₁₋₆alkyl is unsubstituted or substituted with cycloalkyl.

89. A compound of Formula (VII), or a pharmaceutically acceptable salt thereof, having the structure: 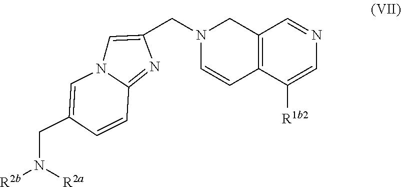
wherein:
R^{1b2} is selected from the group consisting of H, C₁₋₆alkyl, alkenyl, alkynyl, halo, —OR^{1e}, —N(R^{1e})₂, aryl, cycloalkyl, cycloalkenyl, heteroaryl, and heterocyclyl;
wherein the aryl, cycloalkyl, cycloalkenyl, heteroaryl, and heterocyclyl of R^{1b2} is unsubstituted or substituted with one or more substituents selected from the group consisting of C₁₋₆alkyl, halo, —CN, —(CH₂)ₘN(R^{1e})₂, —(CH₂)ₘOH, —C(O)R^{1e}, —C(O)N(R^{1e})₂, —(CH₂)ₘCOOH, aryl, heteroaryl, heterocyclyl, —(CH₂)ₘaryl, cycloalkyl, oxo, —OR^{1e}, —N(R^{1e})₂, —S(O)₂R^{1e}, —S(O)₂N(R^{1e})₂, —NHS(O)₂R^{1e}, —(CH₂)ₘNH(CH₂)ₘS(O)₂R^{1e}, and —NHC(O)R^{1e};
wherein the C₁₋₆alkyl and alkynyl of R^{1b2} is unsubstituted or substituted with one or more substituents selected from the group consisting of aryl, heteroaryl, heterocyclyl, and cycloalkyl;
wherein each R^{1e} is independently H, C₁₋₆alkyl, —(CH₂)ₘCN, —(CH₂)ₘNH₂, cycloalkyl, or heterocyclyl;
wherein each m is independently 1, 2, or 3;
R^{2a} is H or C₁₋₆alkyl;
wherein the C₁₋₆alkyl of Ra is unsubstituted or substituted with one or more substituents selected from the group consisting of halogen, —OH, —CN, —N(R^{2h})₂, alkoxy, cycloalkyl, halocycloalkyl, hydroxycycloalkyl, and heterocyclyl;
R^{2b} is selected from the group consisting of C₁₋₆alkyl, heterocyclyl, cycloalkyl, heterocyclylalkyl, and cycloalkylalkyl;
wherein the C₁₋₆alkyl, heterocyclyl, cycloalkyl, heterocyclylalkyl, and cycloalkylalkyl of R^{2b} is unsubstituted or substituted with one or more substituents selected from the group consisting of C₁₋₆alkyl, C₁₋₆haloalkyl, halogen, —OH, —CN, —N(R^{2h})₂, alkoxy, aryl, haloaryl, cycloalkyl, halocycloalkyl, hydroxycycloalkyl, and heterocyclyl; or Ra and R^{2b} combine to form a 5-8-membered heterocyclyl;
each R^{2h} is independently H, C₁₋₆alkyl, or —(CH₂)ₙOCH₃; and
n is 1, 2, 3, 4, 5, or 6.

90. The compound of Formula (VII) as described in claim 89, or a pharmaceutically acceptable salt thereof, wherein Ra is H.

91. The compound of Formula (VII) as described in claim 89 or 90, or a pharmaceutically acceptable salt thereof, wherein R^{2b} is cycloalkylalkyl.

92. The compound of Formula (VII) as described in claim 89 or 90, or a pharmaceutically acceptable salt thereof, wherein R^{2b} is cycloalkylalkyl substituted with one or more substituents selected from C₁₋₆haloalkyl, halogen, or —CN.

93. The compound of Formula (VII) as described in claim 89, 90, or 92, or a pharmaceutically acceptable salt thereof, wherein —N(R^{2a})(R^{2b}) is selected from:

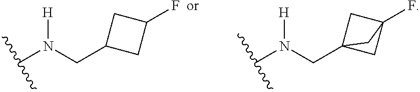

94. The compound of Formula (VII) as described in claim 89, or a pharmaceutically acceptable salt thereof, wherein R^{2a} is C₁₋₆alkyl.

95. The compound of Formula (VII) as described in claim 89 or 94, or a pharmaceutically acceptable salt thereof, wherein R^{2b} is C₁₋₆alkyl.

96. The compound of Formula (VII) as described in claim 89 or 94, or a pharmaceutically acceptable salt thereof, wherein R^{2b} is C₁₋₆alkyl substituted with one or more substituents selected from C₁₋₆haloalkyl, halogen, or —CN.

97. The compound of Formula (VII) as described in claim 89 or 94, or a pharmaceutically acceptable salt thereof, wherein R^{2b} is heterocyclylalkyl.

98. The compound of Formula (VII) as described in claim 89 or 94, or a pharmaceutically acceptable salt thereof, wherein R^{2b} is heterocyclylalkyl substituted with one or more substituents selected from C₁₋₆alkyl, C₁₋₆haloalkyl, halogen, or —CN.

99. The compound of Formula (VII) as described in claim 89 or 94, or a pharmaceutically acceptable salt thereof, wherein R^{2b} is cycloalkylalkyl.

100. The compound of Formula (VII) as described in claim 89 or 94, or a pharmaceutically acceptable salt thereof, wherein R^{2b} is cycloalkylalkyl substituted with one or more substituents selected from C₁₋₆alkyl, C₁₋₆haloalkyl, halogen, or —CN.

101. The compound of Formula (VII) as described in any one of claims 89 or 94-100, or a pharmaceutically acceptable salt thereof, wherein R^{2a} and R^{2b} combine to form a 5-8-membered heterocyclyl.

102. The compound of Formula (VII) as described in claim 89, or a pharmaceutically acceptable salt thereof, wherein —N(R^{2a})(R^{2b}) is selected from:

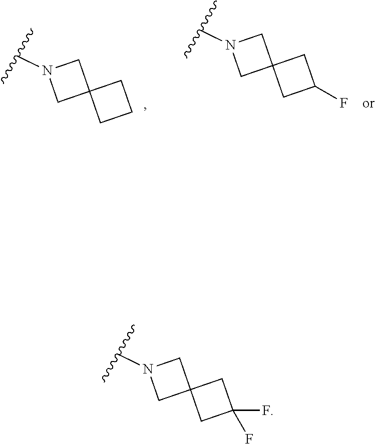

103. The compound of Formula (VII) as described in claim 89, or a pharmaceutically acceptable salt thereof, wherein —N(R^{2a})(R^{2b}) is selected from:

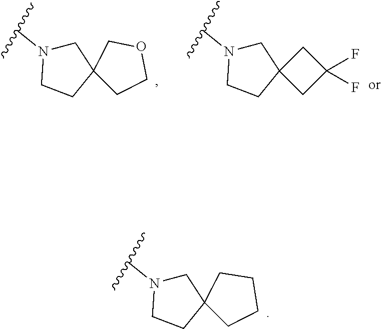 [SMILES: *N1CCC2(CCOC2)C1 ; *N1CCC2(C1)CC(F)(F)C2 ; *N1CCC2(CCCC2)C1]

104. The compound of Formula (VII) as described in claim 89, or a pharmaceutically acceptable salt thereof, wherein —N(R^{2a})(R^{2b}) is selected from:

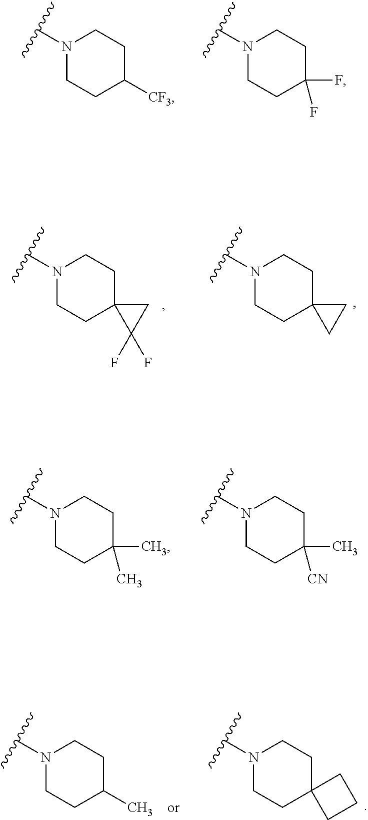

105. The compound of Formula (VII) as described in claim 89, or a pharmaceutically acceptable salt thereof, wherein —N(R^{2a})(R^{2b}) is selected from:

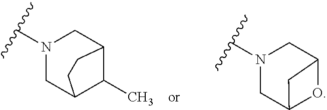

106. The compound of Formula (VII) as described in claim 89, or a pharmaceutically acceptable salt thereof, wherein R^{1b2} is heterocyclyl.

107. The compound of Formula (VII) as described in claim 89, or a pharmaceutically acceptable salt thereof, wherein R^{1b2} is selected from:

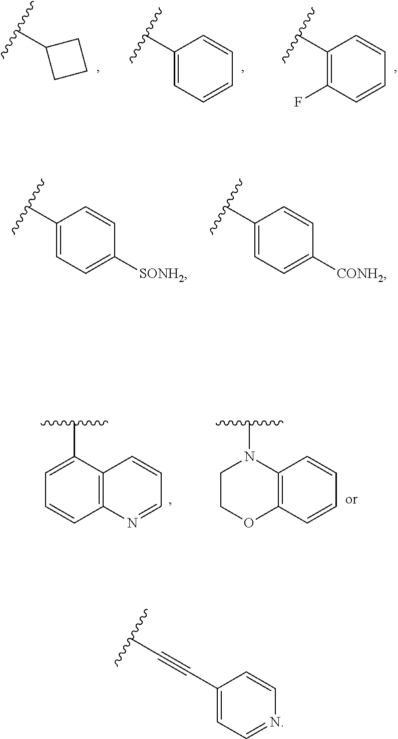

108. The compound of Formula (VII) as described in claim 89, or a pharmaceutically acceptable salt thereof, wherein R^{1b2} is selected from:

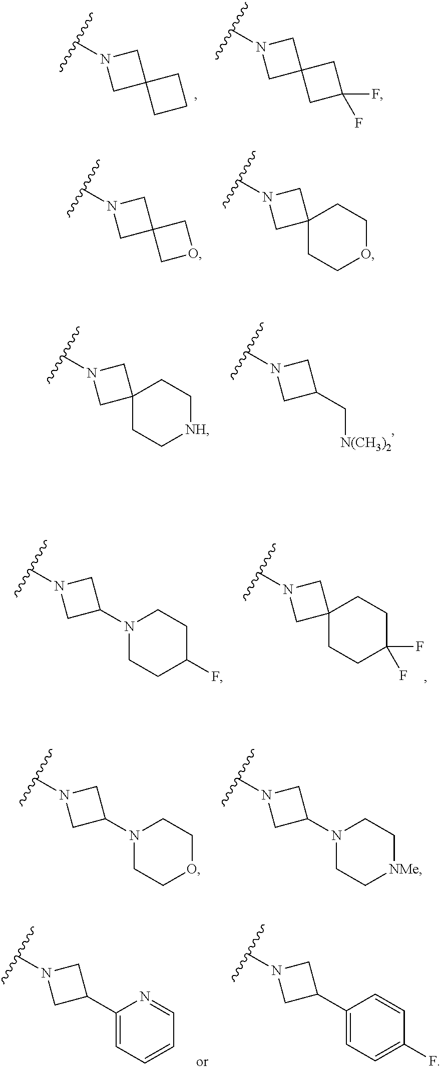

109. The compound of Formula (VII) as described in claim 89, or pharmaceutically acceptable salt thereof, wherein R^{1b2} is selected from:

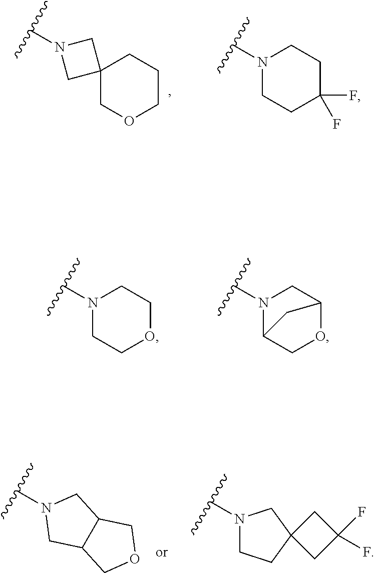 [SMILES: *N1CC2(CCCOC2)C1 ; *N1CCC(F)(F)CC1 ; *N1CCOCC1 ; *N1CC2CC1CO2 ; *N1CC2COCC2C1 ; *N1CCC2(C1)CC(F)(F)C2]

110. A compound, or a pharmaceutically acceptable salt thereof, selected from the group consisting of

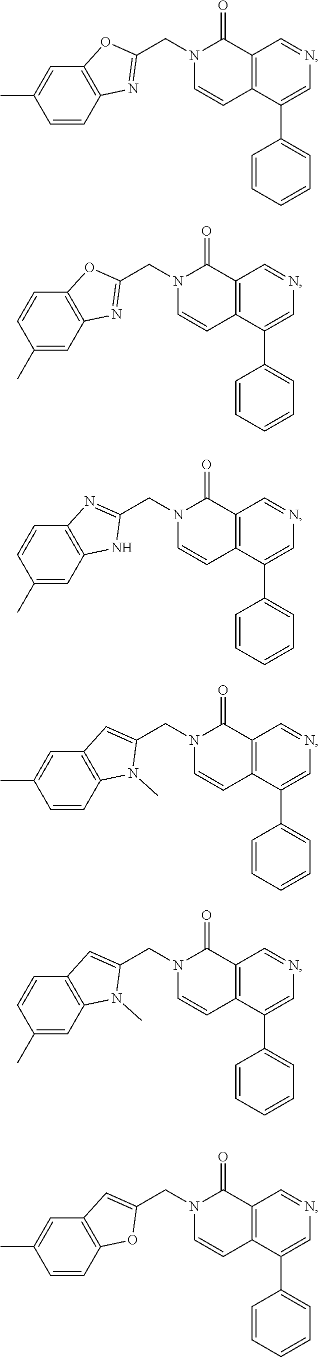

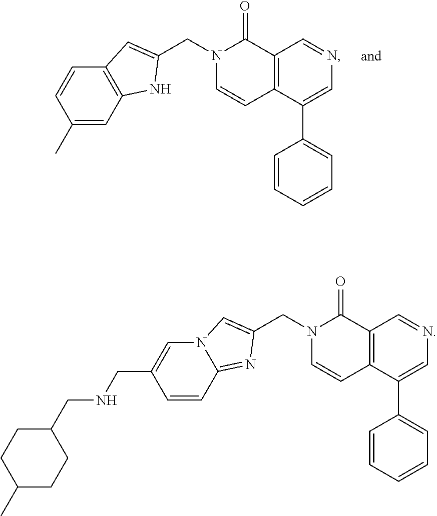 [SMILES: Cc1ccc2cc(Cn3ccc4c(-c5ccccc5)cncc4c3=O)[nH]c2c1 ; "and" ; CC1CCC(CNCc2ccc3nc(Cn4ccc5c(-c6ccccc6)cncc5c4=O)cn3c2)CC1]

111. A compound, or a pharmaceutically acceptable salt thereof, selected from the group consisting of

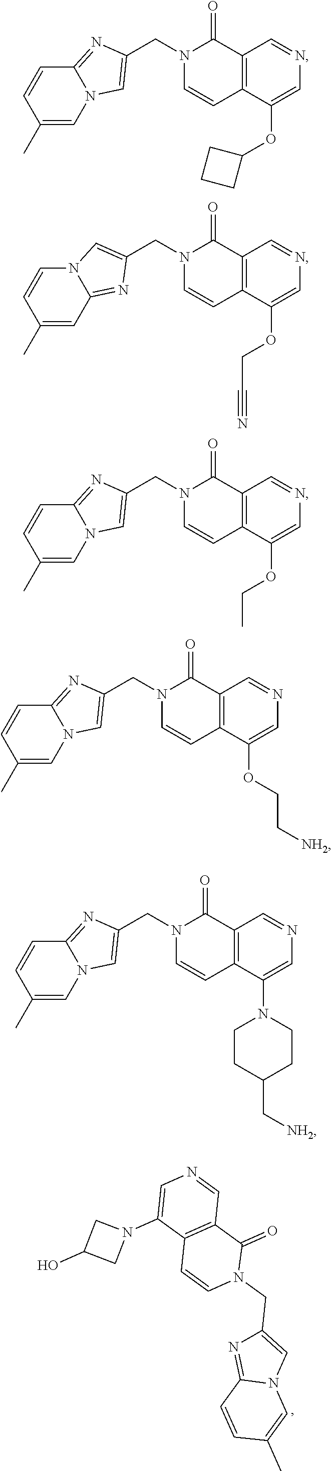

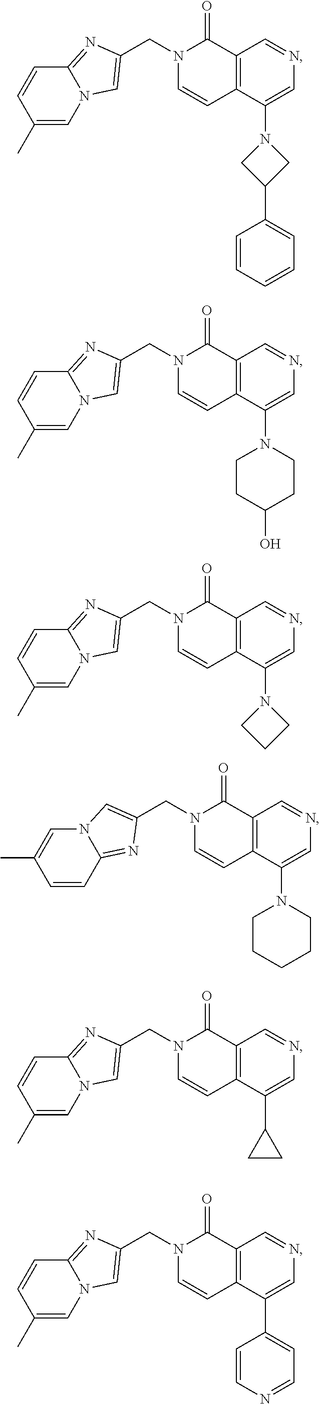

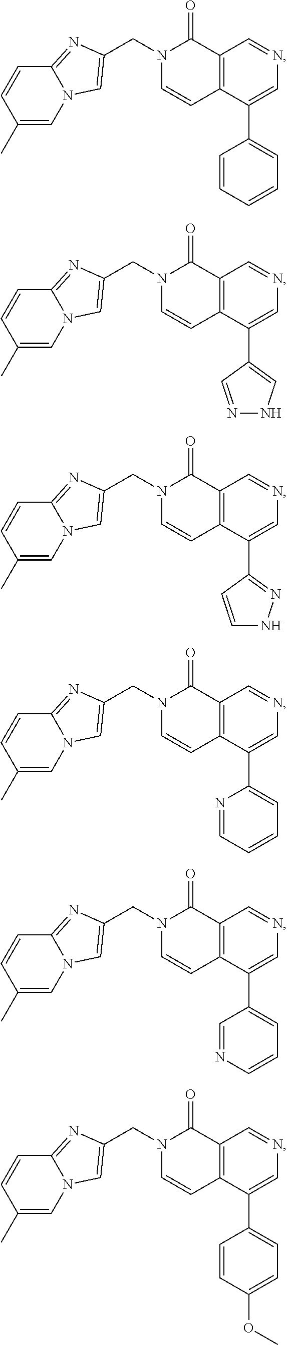 [SMILES: Cc1ccc2nc(Cn3ccc4c(-c5ccccc5)cncc4c3=O)cn2c1 ; Cc1ccc2nc(Cn3ccc4c(-c5cn[nH]c5)cncc4c3=O)cn2c1 ; Cc1ccc2nc(Cn3ccc4c(-c5cc[nH]n5)cncc4c3=O)cn2c1 ; Cc1ccc2nc(Cn3ccc4c(-c5ccccn5)cncc4c3=O)cn2c1 ; Cc1ccc2nc(Cn3ccc4c(-c5cccnc5)cncc4c3=O)cn2c1 ; COc1ccc(-c2cncc3c(=O)n(Cc4cn5cc(C)ccc5n4)ccc23)cc1]

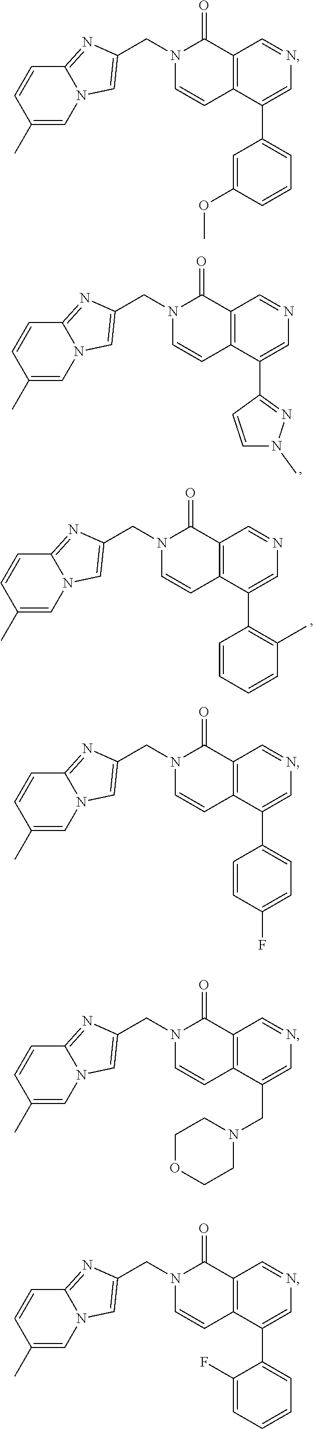

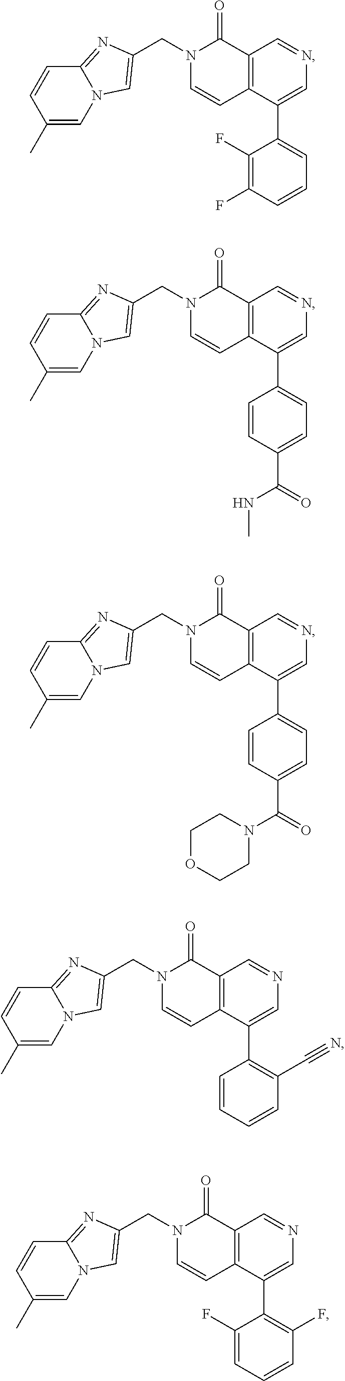 [SMILES: Cc1ccc2nc(Cn3ccc4c(-c5cccc(F)c5F)cncc4c3=O)cn2c1 ; CNC(=O)c1ccc(-c2cncc3c(=O)n(Cc4cn5cc(C)ccc5n4)ccc23)cc1 ; Cc1ccc2nc(Cn3ccc4c(-c5ccc(C(=O)N6CCOCC6)cc5)cncc4c3=O)cn2c1 ; Cc1ccc2nc(Cn3ccc4c(-c5ccccc5C#N)cncc4c3=O)cn2c1 ; Cc1ccc2nc(Cn3ccc4c(-c5c(F)cccc5F)cncc4c3=O)cn2c1]

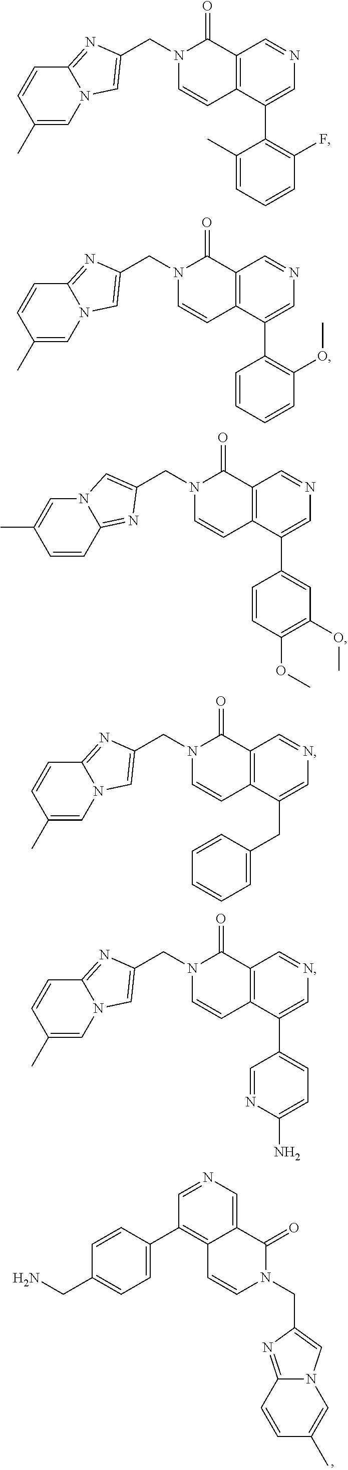

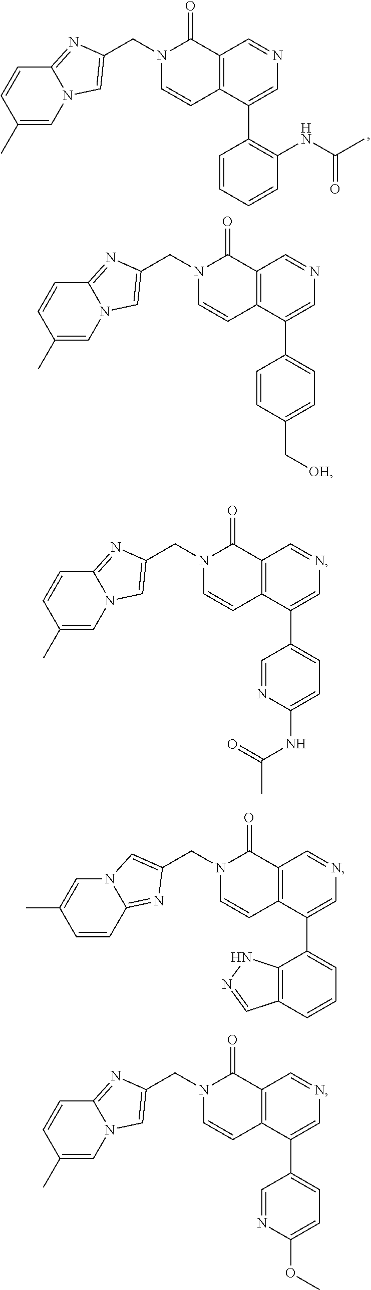

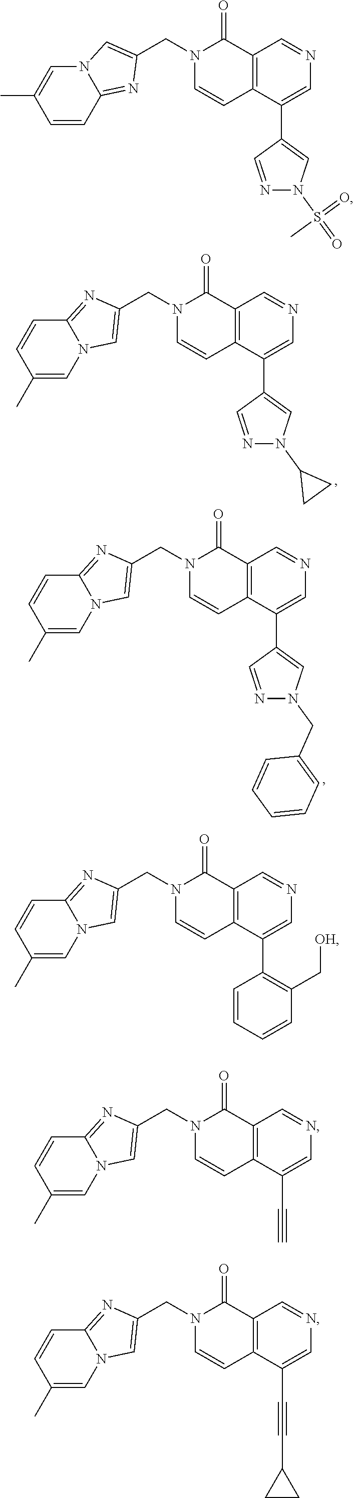 [SMILES: Cc1ccc2nc(Cn3ccc4c(-c5cnn(S(C)(=O)=O)c5)cncc4c3=O)cn2c1 ; Cc1ccc2nc(Cn3ccc4c(-c5cnn(C6CC6)c5)cncc4c3=O)cn2c1 ; Cc1ccc2nc(Cn3ccc4c(-c5cnn(Cc6ccccc6)c5)cncc4c3=O)cn2c1 ; Cc1ccc2nc(Cn3ccc4c(-c5ccccc5CO)cncc4c3=O)cn2c1 ; C#Cc1cncc2c(=O)n(Cc3cn4cc(C)ccc4n3)ccc12 ; Cc1ccc2nc(Cn3ccc4c(C#CC5CC5)cncc4c3=O)cn2c1]

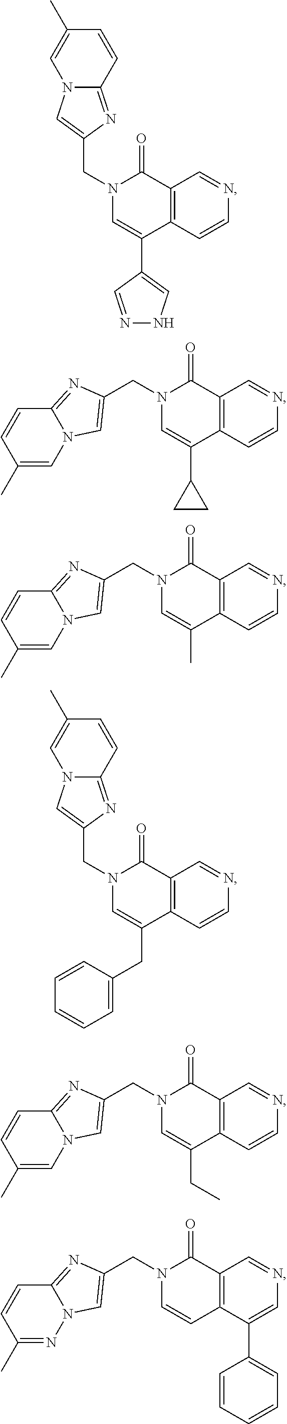

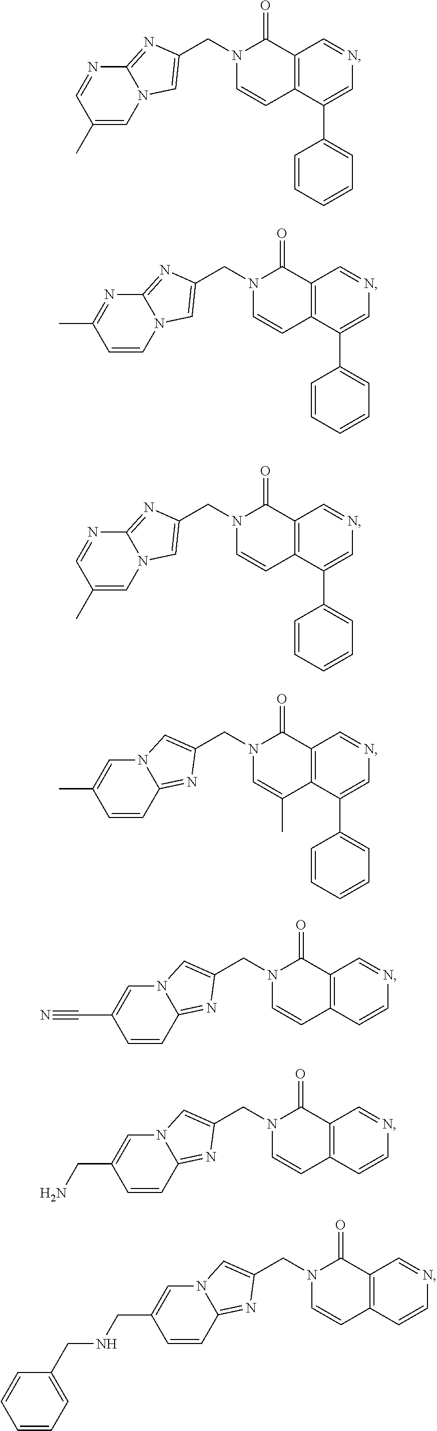

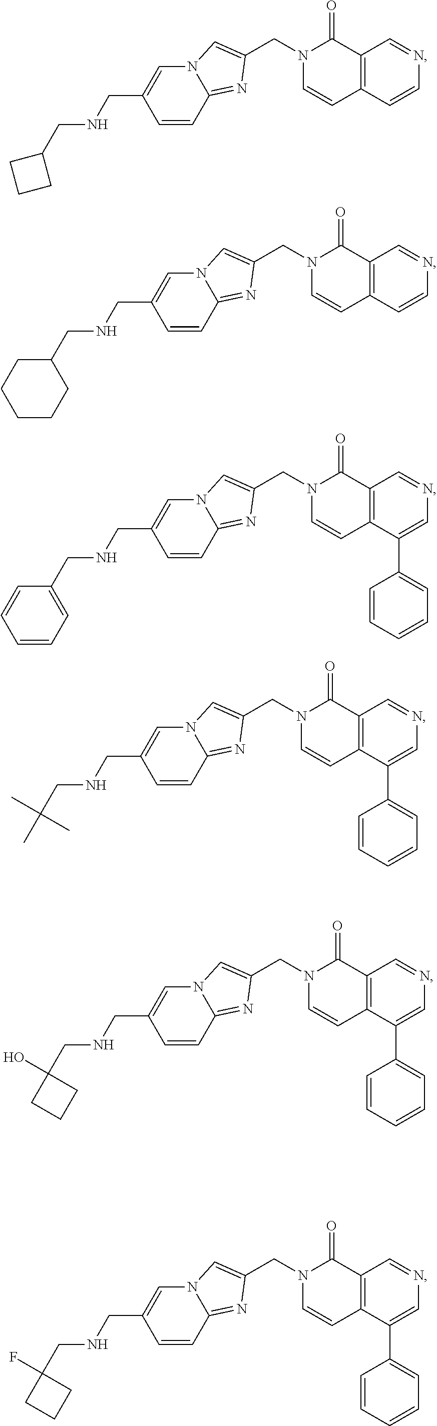 [SMILES: O=c1c2cnccc2ccn1Cc1cn2cc(CNCC3CCC3)ccc2n1 ; O=c1c2cnccc2ccn1Cc1cn2cc(CNCC3CCCCC3)ccc2n1 ; O=c1c2cncc(-c3ccccc3)c2ccn1Cc1cn2cc(CNCc3ccccc3)ccc2n1 ; CC(C)(C)CNCc1ccc2nc(Cn3ccc4c(-c5ccccc5)cncc4c3=O)cn2c1 ; O=c1c2cncc(-c3ccccc3)c2ccn1Cc1cn2cc(CNCC3(O)CCC3)ccc2n1 ; O=c1c2cncc(-c3ccccc3)c2ccn1Cc1cn2cc(CNCC3(F)CCC3)ccc2n1]

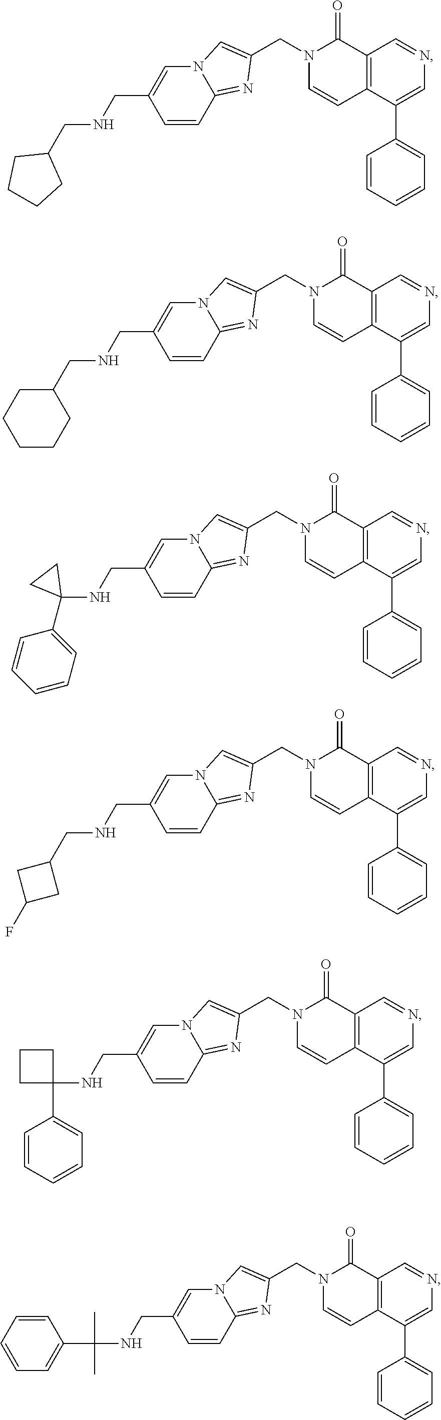 [SMILES: O=c1c2cncc(-c3ccccc3)c2ccn1Cc1cn2cc(CNCC3CCCC3)ccc2n1 ; O=c1c2cncc(-c3ccccc3)c2ccn1Cc1cn2cc(CNCC3CCCCC3)ccc2n1 ; O=c1c2cncc(-c3ccccc3)c2ccn1Cc1cn2cc(CNC3(c4ccccc4)CC3)ccc2n1 ; O=c1c2cncc(-c3ccccc3)c2ccn1Cc1cn2cc(CNCC3CC(F)C3)ccc2n1 ; O=c1c2cncc(-c3ccccc3)c2ccn1Cc1cn2cc(CNC3(c4ccccc4)CCC3)ccc2n1 ; CC(C)(NCc1ccc2nc(Cn3ccc4c(-c5ccccc5)cncc4c3=O)cn2c1)c1ccccc1]

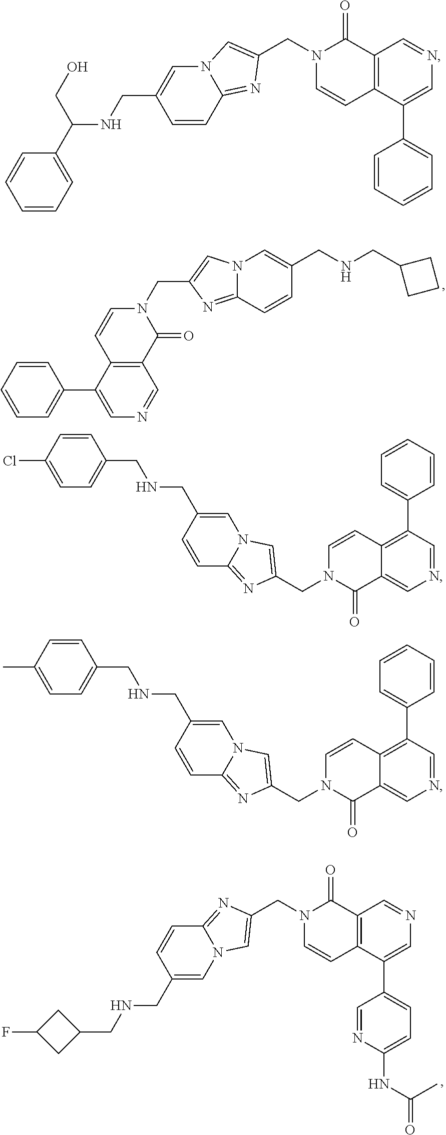

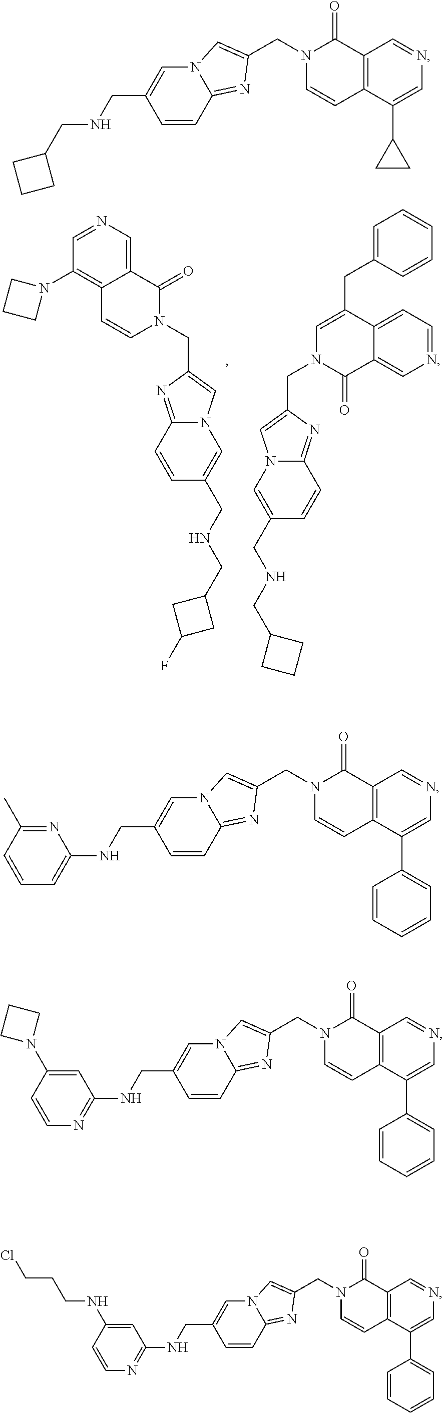

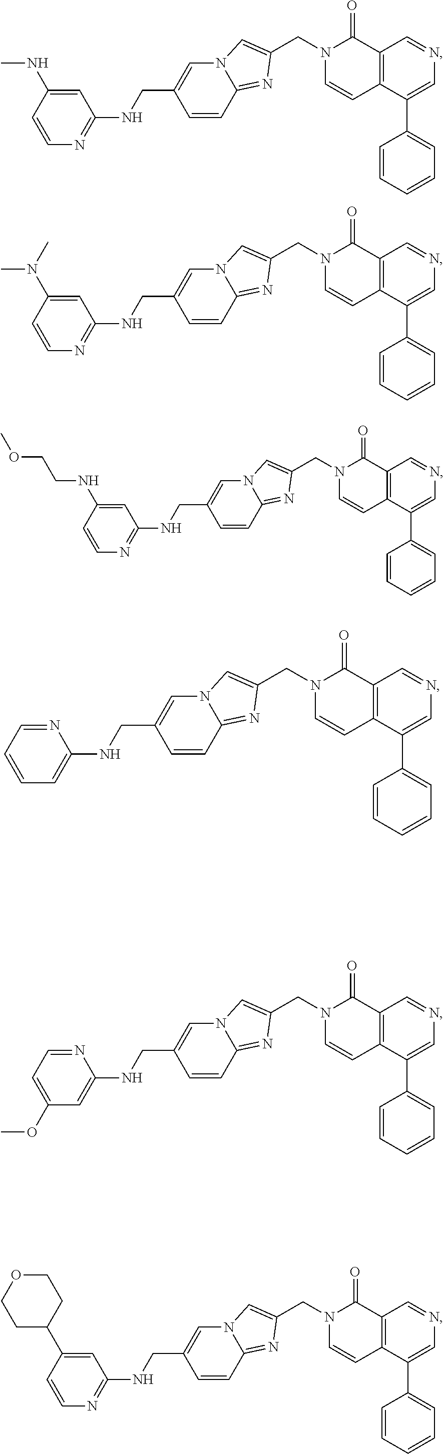

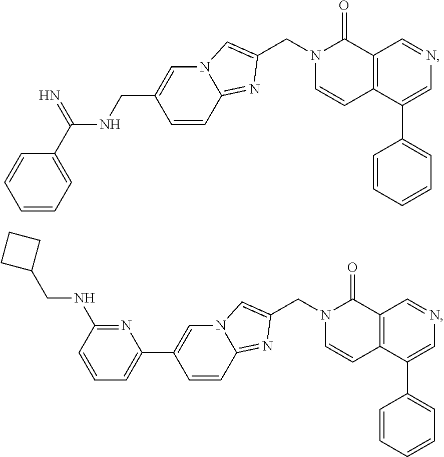

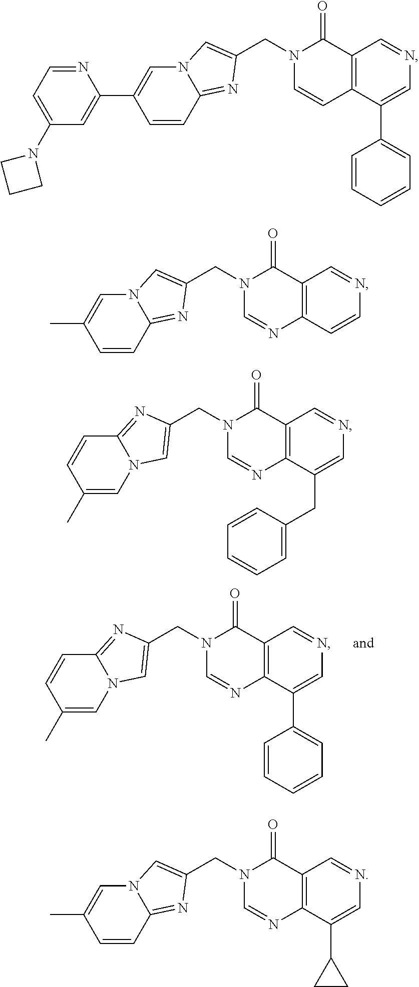

112. A compound, or a pharmaceutically acceptable salt thereof, selected from the group consisting of

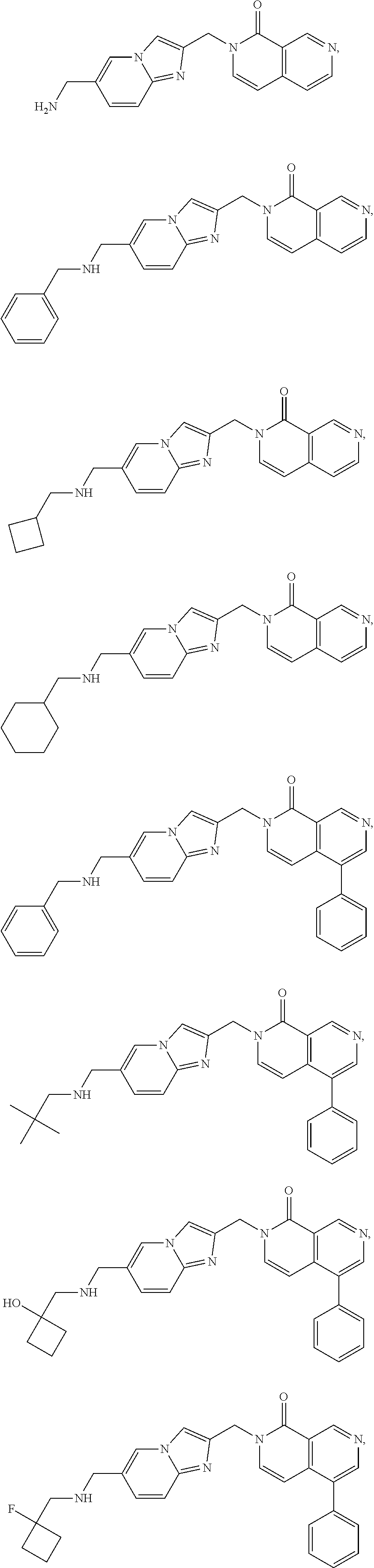

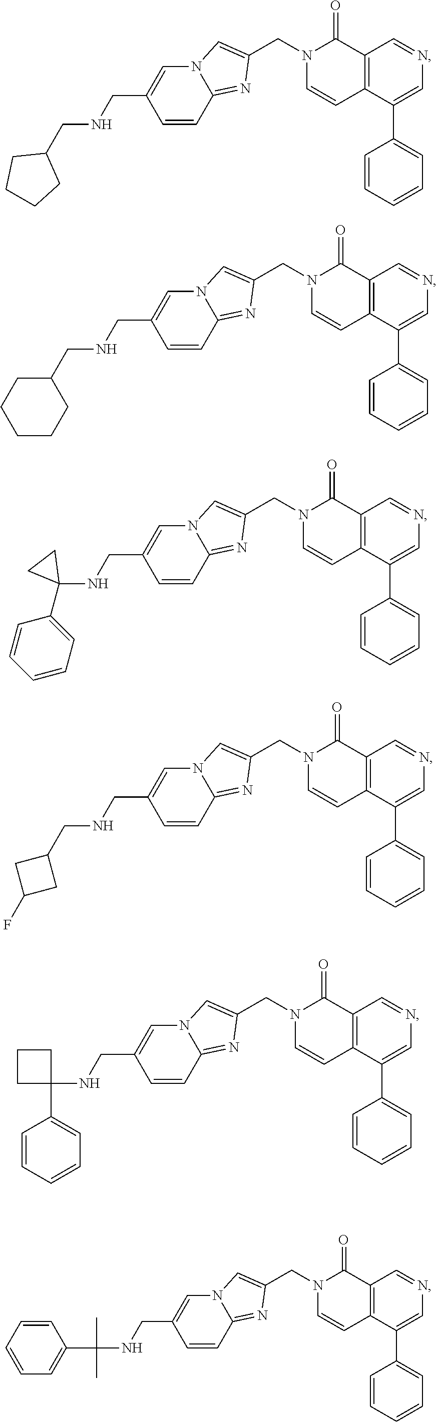 [SMILES: O=c1c2cncc(-c3ccccc3)c2ccn1Cc1cn2cc(CNCC3CCCC3)ccc2n1 ; O=c1c2cncc(-c3ccccc3)c2ccn1Cc1cn2cc(CNCC3CCCCC3)ccc2n1 ; O=c1c2cncc(-c3ccccc3)c2ccn1Cc1cn2cc(CNC3(c4ccccc4)CC3)ccc2n1 ; O=c1c2cncc(-c3ccccc3)c2ccn1Cc1cn2cc(CNCC3CC(F)C3)ccc2n1 ; O=c1c2cncc(-c3ccccc3)c2ccn1Cc1cn2cc(CNC3(c4ccccc4)CCC3)ccc2n1 ; CC(C)(NCc1ccc2nc(Cn3ccc4c(-c5ccccc5)cncc4c3=O)cn2c1)c1ccccc1]

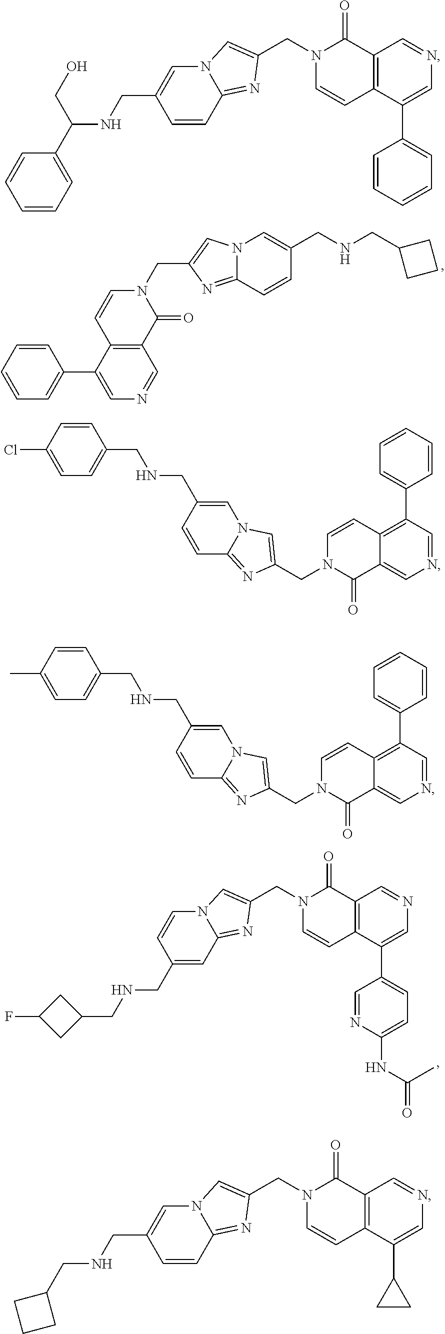

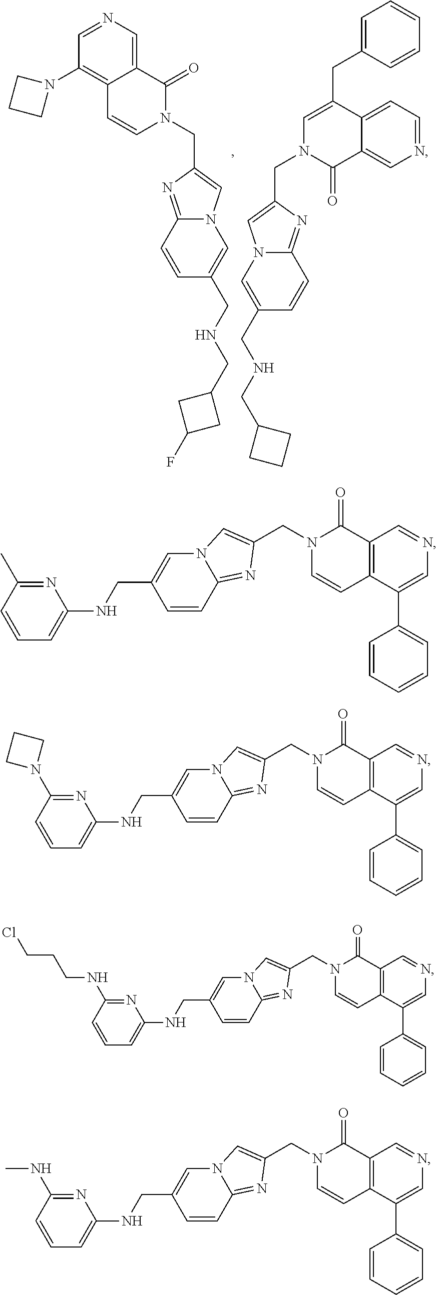

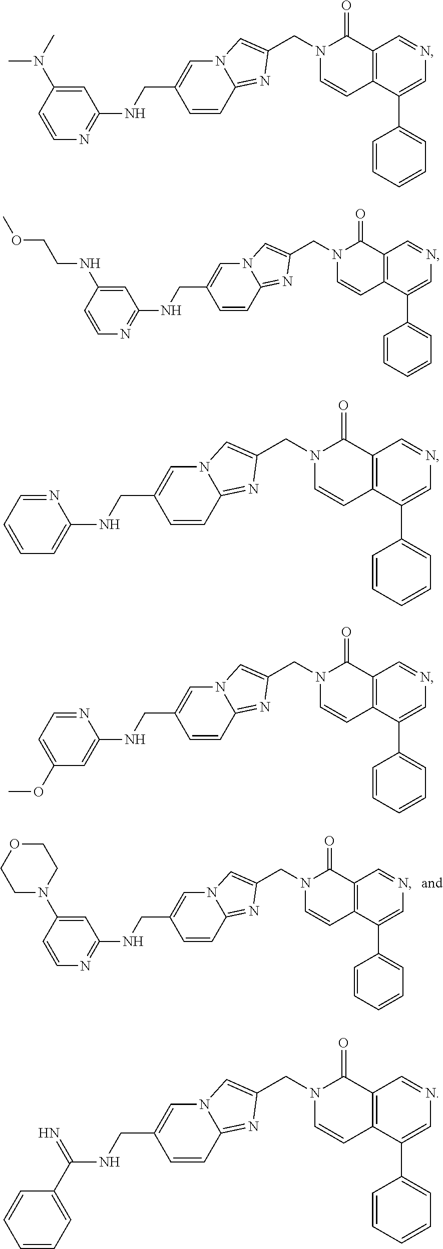 [SMILES: CN(C)c1ccnc(NCc2ccc3nc(Cn4ccc5c(-c6ccccc6)cncc5c4=O)cn3c2)c1 ; COCCNc1ccnc(NCc2ccc3nc(Cn4ccc5c(-c6ccccc6)cncc5c4=O)cn3c2)c1 ; O=c1c2cncc(-c3ccccc3)c2ccn1Cc1cn2cc(CNc3ccccn3)ccc2n1 ; COc1ccnc(NCc2ccc3nc(Cn4ccc5c(-c6ccccc6)cncc5c4=O)cn3c2)c1 ; O=c1c2cncc(-c3ccccc3)c2ccn1Cc1cn2cc(CNc3cc(N4CCOCC4)ccn3)ccc2n1 ; N=C(NCc1ccc2nc(Cn3ccc4c(-c5ccccc5)cncc4c3=O)cn2c1)c1ccccc1]

113. A compound of the structure 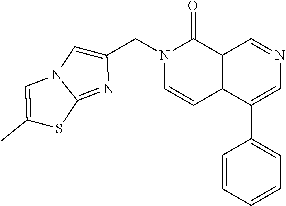 [SMILES: Cc1cn2cc(CN3C=CC4C(c5ccccc5)=CN=CC4C3=O)nc2s1]
or a pharmaceutically acceptable salt thereof.

114. A compound, or a pharmaceutically acceptable salt thereof, selected from the group consisting of

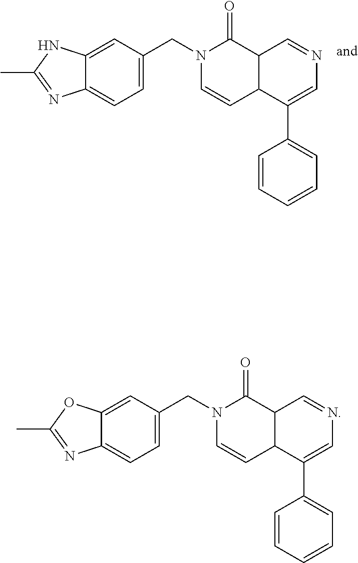 [SMILES: Cc1nc2ccc(CN3C=CC4C(c5ccccc5)=CN=CC4C3=O)cc2[nH]1 ; Cc1nc2ccc(CN3C=CC4C(c5ccccc5)=CN=CC4C3=O)cc2o1]

115. A compound, or a pharmaceutically acceptable salt thereof, selected from the group consisting of

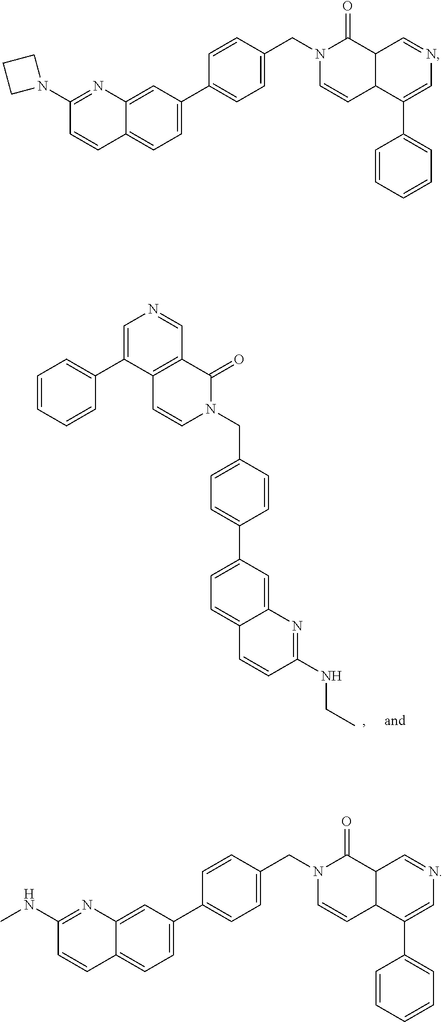

116. A compound, or a pharmaceutically acceptable salt thereof, selected from the group consisting of:
5-{6,6-difluoro-2-azaspiro[3.3]heptan-2-yl}-2-({6-[(4,4-dimethylpiperidin-1-yl)methyl]imidazo[1,2-a]pyridin-2-yl}methyl)-1,2-dihydro-2,7-naphthyridin-1-one;
5-(4,4-difluoropiperidin-1-yl)-2-({6-[(4,4-dimethylpiperidin-1-yl)methyl]imidazo[1,2-a]pyridin-2-yl}methyl)-1,2-dihydro-2,7-naphthyridin-1-one;
2-({6-[(4,4-dimethylpiperidin-1-yl)methyl]imidazo[1,2-a]pyridin-2-yl}methyl)-5-(morpholin-4-yl)-1,2-dihydro-2,7-naphthyridin-1-one;
2-({6-[(4,4-dimethylpiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one;
5-((1 S,4S)-2-oxa-5-azabicyclo[2.2.1]heptan-5-yl)-2-((6-((4,4-dimethylpiperidin-1-yl)methyl)imidazo[1,2-a]pyridin-2-yl)methyl)-2,7-naphthyridin-1(2H)-one;
2-({6-[(4,4-dimethylpiperidin-1-yl)methyl]imidazo[1,2-a]pyridin-2-yl}methyl)-5-[(1R,4R)-2-oxa-5-azabicyclo[2.2.1]heptan-5-yl]-1,2-dihydro-2,7-naphthyridin-1-one;
2-({6-[(4,4-dimethylpiperidin-1-yl)methyl]imidazo[1,2-a]pyridin-2-yl}methyl)-5-{hexahydro-1H-furo[3,4-c]pyrrol-5-yl}-1,2-dihydro-2,7-naphthyridin-1-one;
2-({6-[(4,4-dimethylpiperidin-1-yl)methyl]imidazo[1,2-a]pyridin-2-yl}methyl)-5-[3-(morpholin-4-yl)azetidin-1-yl]-1,2-dihydro-2,7-naphthyridin-1-one;
5-{2,7-diazaspiro[3.5]nonan-2-yl}-2-({6-[(4,4-dimethylpiperidin-1-yl)methyl]imidazo[1,2-a]pyridin-2-yl}methyl)-1,2-dihydro-2,7-naphthyridin-1-one;
5-(3,4-dihydro-2H-1,4-benzoxazin-4-yl)-2-({6-[(4,4-dimethylpiperidin-1-yl)methyl]imidazo[1,2-a]pyridin-2-yl}methyl)-1,2-dihydro-2,7-naphthyridin-1-one;
2-({6-[(4,4-dimethylpiperidin-1-yl)methyl]imidazo[1,2-a]pyridin-2-yl}methyl)-5-phenyl-1,2-dihydro-2,7-naphthyridin-1-one;
2-({6-[(4,4-dimethylpiperidin-1-yl)methyl]imidazo[1,2-a]pyridin-2-yl}methyl)-5-(quinolin-5-yl)-1,2-dihydro-2,7-naphthyridin-1-one;
4-[7-({6-[(4,4-dimethylpiperidin-1-yl)methyl]imidazo[1,2-a]pyridin-2-yl}methyl)-8-oxo-7,8-dihydro-2,7-naphthyridin-4-yl]benzene-1-sulfonamide;
2-((6-((4,4-dimethylpiperidin-1-yl)methyl)imidazo[1,2-a]pyridin-2-yl)methyl)-5-(pyridin-4-ylethynyl)-2,7-naphthyridin-1(2H)-one;
5-{3-[(dimethylamino)methyl]azetidin-1-yl}-2-({6-[(4,4-dimethylpiperidin-1-yl)methyl]imidazo[1,2-a]pyridin-2-yl}methyl)-1,2-dihydro-2,7-naphthyridin-1-one;
2-({6-[(4,4-dimethylpiperidin-1-yl)methyl]imidazo[1,2-a]pyridin-2-yl}methyl)-5-[3-(4-methylpiperazin-1-yl)azetidin-1-yl]-1,2-dihydro-2,7-naphthyridin-1-one;
2-{[6-({6-azaspiro[2.5]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-{[6-({2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-((6-((7-azaspiro[3.5]nonan-7-yl)methyl)imidazo[1,2-a]pyridin-2-yl)methyl)-5-(7-oxa-2-azaspiro[3.5]nonan-2-yl)-2,7-naphthyridin-1(2H)-one;
5-{7-oxa-2-azaspiro[3.5]nonan-2-yl}-2-{[6-({2-oxa-7-azaspiro[4.4]nonan-7-yl}methyl)imidazo[1,2-a]pyridin-2-yl]methyl}-1,2-dihydro-2,7-naphthyridin-1-one;
5-{7-oxa-2-azaspiro[3.5]nonan-2-yl}-2-{[6-({6-oxa-3-azabicyclo[3.1.1]heptan-3-yl}methyl)imidazo[1,2-a]pyridin-2-yl]methyl}-1,2-dihydro-2,7-naphthyridin-1-one;
5-{7-oxa-2-azaspiro[3.5]nonan-2-yl}-2-[(6-{[4-(trifluoromethyl)piperidin-1-yl]methyl}imidazo[1,2-a]pyridin-2-yl)methyl]-1,2-dihydro-2,7-naphthyridin-1-one;
2-{[6-({8-methyl-3,8-diazabicyclo[3.2.1]octan-3-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
4-methyl-1-({2-[(5-{7-oxa-2-azaspiro[3.5]nonan-2-yl}-1-oxo-1,2-dihydro-2,7-naphthyridin-2-yl)methyl]imidazo[1,2-a]pyridin-6-yl}methyl)piperidine-4-carbonitrile;
2-{[6-({6,6-difluoro-2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-{[6-({6-fluoro-2-azaspiro[3.3]heptan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-{[6-({2-azaspiro[4.4]nonan-2-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-({6-[(4-methylpiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one;
2-({6-[(4,4-difluoropiperidin-1-yl)methyl]imidazo[1,2-a]pyridin-2-yl}methyl)-5-{7-oxa-2-azaspiro[3.5]nonan-2-yl}-1,2-dihydro-2,7-naphthyridin-1-one;
2-{[6-({2,2-difluoro-6-azaspiro[3.4]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
2-{[6-({1,1-difluoro-6-azaspiro[2.5]octan-6-yl}methyl)imidazo[1,2-a]pyridin-2-yl]methyl}-5-{7-oxa-2-azaspiro[3.5]nonan-2-yl}-1,2-dihydro-2,7-naphthyridin-1-one;
5-{6,6-difluoro-2-azaspiro[3.3]heptan-2-yl}-2-{[6-({[(3-fluorocyclobutyl)methyl]amino}methyl)imidazo[1,2-a]pyridin-2-yl]methyl}-1,2-dihydro-2,7-naphthyridin-1-one;
2-{[6-({[(3-fluorocyclobutyl)methyl]amino}methyl)imidazo[1,2-a]pyridin-2-yl]methyl}-5-{2-oxa-6-azaspiro[3.3]heptan-6-yl}-1,2-dihydro-2,7-naphthyridin-1-one;
2-{[6-({[(3-fluorocyclobutyl)methyl]amino}methyl)imidazo[1,2-a]pyridin-2-yl]methyl}-5-{7-oxa-2-azaspiro[3.5]nonan-2-yl}-1,2-dihydro-2,7-naphthyridin-1-one;
2-{[6-({[(3-fluorocyclobutyl)methyl]amino}methyl)imidazo[1,2-a]pyridin-2-yl]methyl}-5-{6-oxa-2-azaspiro[3.5]nonan-2-yl}-1,2-dihydro-2,7-naphthyridin-1-one;
5-{7,7-difluoro-2-azaspiro[3.5]nonan-2-yl}-2-{[6-({[(3-fluorocyclobutyl)methyl]amino}methyl)imidazo[1,2-a]pyridin-2-yl]methyl}-1,2-dihydro-2,7-naphthyridin-1-one;
5-{2,2-difluoro-6-azaspiro[3.4]octan-6-yl}-2-{[6-({[(3-fluorocyclobutyl)methyl]amino}methyl)imidazo[1,2-a]pyridin-2-yl]methyl}-1,2-dihydro-2,7-naphthyridin-1-one;
2-{[6-({[(3-fluorocyclobutyl)methyl]amino}methyl)imidazo[1,2-a]pyridin-2-yl]methyl}-5-[3-(4-fluoropiperidin-1-yl)azetidin-1-yl]-1,2-dihydro-2,7-naphthyridin-1-one;
2-{[6-({[(3-fluorocyclobutyl)methyl]amino}methyl)imidazo[1,2-a]pyridin-2-yl]methyl}-5-[3-(pyridin-4-yl)azetidin-1-yl]-1,2-dihydro-2,7-naphthyridin-1-one;
2-{[6-({[(3-fluorocyclobutyl)methyl]amino}methyl)imidazo[1,2-a]pyridin-2-yl]methyl}-5-[3-(4-fluorophenyl)azetidin-1-yl]-1,2-dihydro-2,7-naphthyridin-1-one;
2-((6-((((3-fluorobicyclo[1.1.1]pentan-1-yl)methyl)amino)methyl)imidazo[1,2-a]pyridin-2-yl)methyl)-5-(6-oxa-2-azaspiro[3.4]octan-2-yl)-2,7-naphthyridin-1(2H)-one;
2-[(6-{[((3-fluorobicyclo[1.1.1]pentan-1-yl)methyl)amino]methyl}imidazo[1,2-a]pyridin-2-yl)methyl]-5-{7-oxa-2-azaspiro[3.5]nonan-2-yl}-1,2-dihydro-2,7-naphthyridin-1-one;
2-[(6-{[((3-fluorobicyclo[1.1.1]pentan-1-yl)methyl)amino]methyl}imidazo[1,2-a]pyridin-2-yl)methyl]-5-{6-oxa-2-azaspiro[3.5]nonan-2-yl}-1,2-dihydro-2,7-naphthyridin-1-one;
2-[(6-{[({3-fluorobicyclo[1.1.1]pentan-1-yl}methyl)amino]methyl}imidazo[1,2-a]pyridin-2-yl)methyl]-5-(2-fluorophenyl)-1,2-dihydro-2,7-naphthyridin-1-one;
4-{7-[(6-{[({3-fluorobicyclo[1.1.1]pentan-1-yl}methyl)amino]methyl}imidazo[1,2-a]pyridin-2-yl)methyl]-8-oxo-7,8-dihydro-2,7-naphthyridin-4-yl}benzamide;
5-cyclobutyl-2-{[6-({[(3-fluorocyclobutyl)methyl]amino}methyl)imidazo[1,2-a]pyridin-2-yl]methyl}-1,2-dihydro-2,7-naphthyridin-1-one; and
3-[(6-{[({3-fluorobicyclo[1.1.1]pentan-1-yl}methyl)amino]methyl}imidazo[1,2-a]pyridin-2-yl)methyl]-8-{2-oxa-6-azaspiro[3.3]heptan-6-yl}pyrido[3,4-d]pyridazin-4-one.

117. A pharmaceutical composition comprising a compound of anyone of claims 1-116, or a pharmaceutically acceptable salt thereof, and a pharmaceutically acceptable carrier.

118. A compound of any one of claims 1-116, or a pharmaceutically acceptable salt thereof, for use in medicine.

119. A compound of any one of claims 1-116, or a pharmaceutically acceptable salt thereof, for use in the treatment of a disorder that is responsive to modulation or inhibition of METTL3.

120. Use of a compound of any one of claims 1-116, or a pharmaceutically acceptable salt thereof, in the treatment of a disorder that is responsive to modulation or inhibition of METTL3.

121. A compound of any one of claims 1-116, or a pharmaceutically acceptable salt thereof, for use in the manufacture of a medicament for the treatment of a disorder that is responsive to modulation or inhibition of METTL3.

122. A method of treating a disorder in a subject in need thereof, wherein the disorder is mediated by METTL3, comprising administering to the subject an effective amount of a compound of any one of claims 1-116, or a pharmaceutically acceptable salt thereof.

123. The compound of claim 119 or 121, the use of claim 120, or the method of claim 122, wherein the disease, disorder, or condition is selected from the group consisting of neurological diseases, viral diseases, cancer, and pre-cancerous syndromes.

124. The compound of claim 119 or 121, the use of claim 120, or the method of claim 122 or 123, wherein the disorder is cancer and the cancer is selected from the group consisting of blood cancer, pancreatic cancer, gastric cancer, colorectal cancer, ovarian cancer, endometrial cancer, bladder cancer, breast cancer, brain cancer, melanoma, lung cancer, and liver cancer.

125. The compound of claim 119 or 121, the use of claim 120, or the method of any one of claims 121-123, wherein the disorder is leukemia.

126. The compound of claim 119 or 121, the use of claim 120, or the method of any one of claims 122-125, wherein the disorder is acute myeloid leukemia(AML).
